(19) United States
(12) Patent Application Publication
(10) Pub. No.: US 2025/0280696 A1
(43) Pub. Date: Sep. 4, 2025

(54) FORMULATIONS AND USES THEREOF IN OPTOELECTRONIC FIELD

(71) Applicant: Zhejiang Brilliant Optoelectronic Technology Co.,Ltd., Taizhou (CN)

(72) Inventors: Junyou PAN, Taizhou (CN); Juye ZHU, Taizhou (CN); Markus MUEHLINGHAUS, Taizhou (CN); Klaus MUELLEN, Taizhou (CN)

(73) Assignee: Zhejiang Brilliant Optoelectronic Technology Co.,Ltd., Taizhou (CN)

(21) Appl. No.: 19/209,567

(22) Filed: May 15, 2025

Related U.S. Application Data
(63) Continuation of application No. PCT/CN2023/132404, filed on Nov. 17, 2023.

(30) Foreign Application Priority Data
Nov. 17, 2022 (CN) 202211441864.8

(51) Int. Cl. H10K 59/38; C07C 211/57; C09K 11/06; G02F 1/061; H10K 85/60; H10K 102/00

(52) U.S. Cl. CPC H10K 59/38; C07C 211/57; C09K 11/06; G02F 1/061; G02F 2202/02; H10K 85/622; H10K 85/633; H10K 85/636; H10K 85/654; H10K 85/6572; H10K 85/6574; H10K 85/6576; H10K 85/658; H10K 2102/3026

(57) ABSTRACT

Disclosed are formulations including an organic compound H of formula (I), an emitter E. Also provided are organic functional films containing the formulations, or formed by using the formulations. Further provided are optoelectronic devices containing the formulations. Preferably, the optoelectronic device according to the present disclosure is an organic light emitting device containing a color conversion layer. The color conversion layer contains the formulation, in which the organic compound H absorbs light of an excitation light source and transfers energy to the light-emitting body E, and the light-emitting body E absorbs the energy of the organic compound H and then emits emergent light having a narrow full width at half maximum. These light-emitting devices having narrow full widths at half maximum can be used for manufacturing display devices having high color gamut.

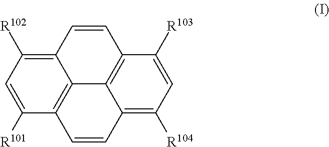

### CROSS-REFERENCE TO RELATED APPLICATIONS

The present application is a continuation of International Application No. PCT/CN2023/132404, filed on Nov. 17, 2023, which claims priority to Chinese Patent Application No. 202211441864.8, filed on Nov. 17, 2022. All of the aforementioned applications are incorporated herein by reference in their entireties.

### TECHNICAL FIELD

The present disclosure relates to the field of organic optoelectronic material and device technology, and in particularly to a formulation, an organic functional film, an optoelectronic device, an organic light-emitting device, and the applications thereof in the optoelectronic field.

### BACKGROUND

According to the principles of colorimetry, the narrower the full width at half maximum (FWHM) of the lights perceived by the human eyes is, the higher the color purity, and the more vivid the color display would be. Display devices with narrow-FWHM red, green and blue primary light are able to show vivid views with high color gamut and high visual quality.

The current mainstream full-color displays are achieved mainly in two ways. The first method is to actively emit red, green and blue lights, typically such as red-green-blue-organic light-emitting diode (RGB-OLED) display. The current mature technology is to fabricate light-emitting devices with three colors by vacuum evaporation with fine metal masks, which is complex, at high cost and difficult to achieve high-resolution display over 600 ppi. The second method is using color converters to convert the single-color light from the light-emitting devices into different colors, thereby achieving a full-color display. For example, Samsung combines blue organic light-emitting diodes (OLEDs) with red and green quantum dots (QD) films as the color converters. In this case, the fabrication of the light-emitting devices is much simpler, and thus higher yield. Furthermore, the manufacture of the color converters can be achieved by different technologies, such as vacuum evaporation, ink-jet printing, transfer printing and photolithography, etc., applicable to a variety of display products with very different resolution requirements from low resolution large-size television (TV) (around only 50 ppi) to high resolution silicon-based micro-display (over 3000 ppi).

Currently, there are mainly two types of color conversion materials used in mainstream color converters. The first one is an inorganic nanocrystal, commonly known as a quantum dot, which is a nanoparticle (especially is a quantum dot) of an inorganic semiconductor material (InP, CdSe, CdS, ZnSe, etc.) with a diameter of 2 nm to 8 nm. Limited by the current synthesis and separation technology of quantum dots, the FWHMs of CD-containing quantum dots typically range from 25 nm to 40 nm, which meet the display requirements of NTSC for color purity.

Meanwhile, Cd-free quantum dots generally come with larger FWHMs of 35 nm to 75 nm. In addition, the extinction coefficient is generally low, requiring thicker films, the typical 10 m or more is needed to achieve complete absorption of blue light, which is a great challenge for mass production processes, especially for Samsung's technology of combing blue OLED with red-green quantum dots. The second one is an organic dye, comprising various organic conjugated small molecules with chromophores. This organic dye generally has high extinction coefficient, but the intra-molecular thermal relaxation and the large vibration energy are always non-negligible, leading to the large FWHM (typically over 60 nm) of its emission spectrum.

The present inventors have disclosed a color converter with host-dopant combinations in the previous patent application, where the host possesses a high extinction coefficient and the dopant exhibits narrow emission spectrum, this provides support for developing noval thin color converters. However, the stabilities (including photostability and thermal stability) of the host material still need to be greatly improved.

### SUMMARY

In one aspect, the present disclosure provides a formulation comprising an organic compound H of formula (I), an emitter E, where 1) the emission spectrum of the organic compound H is on the short wavelength side of the absorption spectrum of the emitter E, and at least partially overlaps with the absorption spectrum of the emitter E; 2) the FWHM of the emission spectrum of the emitter E≤55 nm;

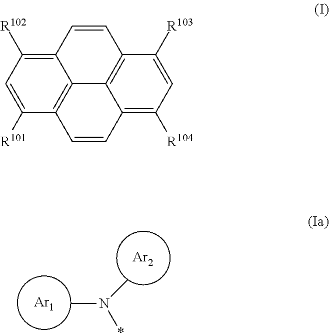

Where each of R¹⁰¹ to R¹⁰⁴ is independently selected from —H, -D, a C₁-C₂₀ linear alkyl group, a C₁-C₂₀ linear alkenyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents or any combination thereof, where at least one of R¹⁰¹-R¹⁰⁴ is a formula (Ia), where each of Ar₁ and Ar² is a substituted/unsubstituted aromatic or heteroaromatic group containing 5 to 24 ring atoms, each * independently represents an attachment site connecting a pyrene.

In addition or alternatively, the formulation further comprises at least one organic resin and/or a solvent.

In another aspect, the present disclosure also provides an organic functional film comprising a formulation as described herein, or formed by using a formulation as described herein.

In yet another aspect, the present disclosure further provides an optoelectronic device comprising a formulation or an organic functional film as described herein.

In addition or alternatively, the optoelectronic device is an organic light-emitting device comprising a first electrode, an organic light-emitting layer, a second electrode, a color conversion layer, and an encapsulation layer in sequence from bottom to top, the second electrode is at least partially transparent, the color conversion layer at least partially absorbs the light emitted by the organic light-emitting layer through the second electrode, the color conversion layer comprises a formulation as described herein, or formed by using a formulation as described herein.

Beneficial effect: in the formulation as described herein, 1) the organic compound H exhibits high stability, especially photostability; 2) the absorption and emission spectrums of the organic compound H in the film are less or substantially not red-shifted comparing to that in the toluene.

### BRIEF DESCRIPTION OF THE DRAWINGS

FIG. 1 shows a schematic view of a red, green and blue (RGB) three-color display device.

FIG. 2 shows the absorption and emission spectrum of the compound 1 in the toluene.

FIG. 3 shows the absorption and emission spectrum of the compound 1 in the film.

FIG. 4 shows the absorption and emission spectrum of the compound 2 in the toluene.

FIG. 5 shows the absorption and emission spectrum of the compound 2 in the film.

FIG. 6 shows the absorption and emission spectrum of the compound 4 in the toluene.

FIG. 7 shows the absorption and emission spectrum of the compound 4 in the film.

FIG. 8 shows the absorption and emission spectrum of the compound 6 in the toluene.

FIG. 9 shows the absorption and emission spectrum of the compound 6 in the film.

FIG. 10 shows the absorption and emission spectrum of the compound 7 in the toluene.

FIG. 11 shows the absorption and emission spectrum of the compound 7 in the film.

FIG. 12 shows the absorption and emission spectrum of the compound 10 in the toluene.

FIG. 13 shows the absorption and emission spectrum of the compound 10 in the film.

FIG. 14 shows the absorption and emission spectrum of the compound 11 in the toluene.

FIG. 15 shows the absorption and emission spectrum of the compound 11 in the film.

FIG. 16 shows the absorption and emission spectrum of the compound 16 in the toluene.

FIG. 17 shows the absorption and emission spectrum of the compound 16 in the film.

FIG. 18 shows the absorption and emission spectrum of the compound 17 in the toluene.

FIG. 19 shows the absorption and emission spectrum of the compound 17 in the film.

FIG. 20 shows the absorption and emission spectrum of the compound 20 in the toluene.

FIG. 21 shows the absorption and emission spectrum of the compound 20 in the film.

FIG. 22 shows the absorption and emission spectrum of the compound 21 in the toluene.

FIG. 23 shows the absorption and emission spectrum of the compound 21 in the film.

FIG. 24 shows the absorption and emission spectrum of the compound 22 in the toluene.

FIG. 25 shows the absorption and emission spectrum of the compound 22 in the film.

FIG. 26 shows the absorption and emission spectrum of the compound 23 in the toluene.

FIG. 27 shows the absorption and emission spectrum of the compound 23 in the film.

FIG. 28 shows the absorption and emission spectrum of the compound 24 in the toluene.

FIG. 29 shows the absorption and emission spectrum of the compound 24 in the film.

FIG. 30 shows the absorption and emission spectrum of the compound 25 in the toluene.

FIG. 31 shows the absorption and emission spectrum of the compound 25 in the film.

FIG. 32 shows the absorption and emission spectrum of the compound 26 in the toluene.

FIG. 33 shows the absorption and emission spectrum of the compound 26 in the film.

FIG. 34 shows the absorption and emission spectrum of the E1 in the toluene.

FIG. 35 shows the absorption and emission spectrum of the E2 in the toluene.

FIG. 36 shows the absorption and emission spectrum of the E3 in the toluene.

FIG. 37 shows the absorption and emission spectrum of the E4 in the toluene.

FIG. 38 shows the absorption and emission spectrum of the comparative compound 1 in the toluene.

FIG. 39 shows the absorption and emission spectrum of the comparative compound 1 in the film.

FIG. 40 shows the absorption decay of the compound 1, compound 2, compound 4, compound 6, compound 7, compound 10, compound 11, compound 16, compound 17, compound 20, compound 22, compound 23, compound 24, compound 26, and comparative compound 2 after UV irradiation in the toluene.

FIG. 41 shows the luminance decay of the films of the compound 10 and the comparative compound 1 after blue-light illumination.

FIG. 42 shows the spectrum of the resin film of the green CCL combined with the blue top-emitting OLED.

FIG. 43 shows the spectrum of the resin film of the green CCL combined with the blue bottom-emitting OLED.

FIG. 44 shows the spectrum of the resin film of the red CCL combined with the blue top-emitting OLED.

FIG. 45 shows the spectrum of the evaporated film of the green CCL combined with the blue top-emitting OLED.

### DETAILED DESCRIPTION OF THE EMBODIMENTS

The present disclosure provides a formulation and the application thereof in the optoelectronic field. In order to facilitate understanding of the present disclosure, the present disclosure will be described in detail below with reference to the accompanying drawings, in which the preferred embodiments of the present disclosure are shown. The present disclosure may be embodied in many different forms and is not limited to the embodiments described herein. Rather, these embodiments are provided so that the understanding of the disclosure of the present disclosure will be more thorough.

Unless otherwise defined, all technical and scientific terms used herein have the same meaning as commonly understood by one of ordinary skill in the art belonging to the present disclosure. The terms used herein in the description of the present disclosure are used only for the purpose of describing specific embodiments and are not intended to be limiting of the present disclosure. As used herein, the term “and/or” includes any and all combinations of one or more of the relevant listed items.

As used herein, the terms “host material”, “matrix material” have the same meaning, and they are interchangeable with each other.

As used herein, the terms “metal organic clathrate”, “metal organic complexe”, and “organomentallic complexe” have the same meaning, and they are interchangeable with each other.

As used herein, the terms “formulation”, “printing ink”, and “ink” have the same meaning, and they are interchangeable with each other.

In one aspect, the present disclosure provides a formulation comprising an organic compound H of formula (I), an emitter E, where 1) the emission spectrum of the organic compound H is on the short wavelength side of the absorption spectrum of the emitter E, and at least partially overlaps with the absorption spectrum of the emitter E; 2) the FWHM of the emission spectrum of the emitter E≤55 nm;

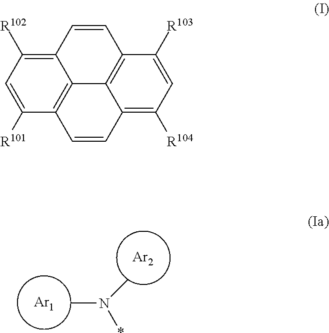

Where each of R¹⁰¹ to R¹⁰⁴ is independently selected from —H, -D, a C₁-C₂₀ linear alkyl group, a C₁-C₂₀ linear alkenyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents or any combination thereof, where at least one of R¹⁰¹-R¹⁰⁴ is a formula (Ia), where each of Ar₁ and Ar² is a substituted/unsubstituted aromatic or heteroaromatic group containing 5 to 24 ring atoms, each * independently represents an attachment site connecting a pyrene.

In some embodiments, at least one of R¹⁰¹-R¹⁰⁴ is selected from one of formulas (Ia-1)-(Ia-4):

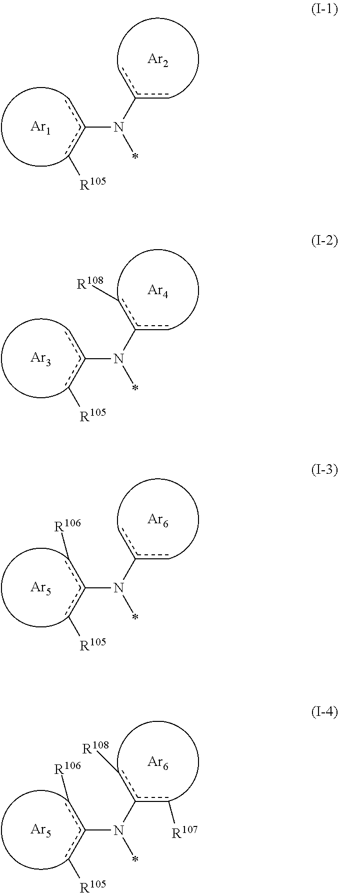

Where *, Ar¹, and Ar² are identically defined as described herein; each of R¹⁰⁵ to R¹⁰⁸ is a substituent, and at each occurrence is independently selected from a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, or any combination thereof.

In some embodiments, each of R¹⁰⁵ to R¹⁰⁸ at each occurrence is independently selected from a C₁-C₁₀ linear alkyl group, a C₁-C₁₀ linear haloalkyl group, a C₁-C₁₀ linear alkoxy group, a C₁-C₁₀ linear thioalkoxy group, a C₃-C₁₀ branched/cyclic alkyl group, a C₃-C₁₀ branched/cyclic haloalkyl group, a C₃-C₁₀ branched/cyclic alkoxy group, a C₃-C₁₀ branched/cyclic thioalkoxy group, a C₃-C₁₀ branched/cyclic silyl group, a C₁-C₁₀ ketone group, a C₂-C₁₀ alkoxycarbonyl group, a C₆-C₁₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 20 ring atoms, an aryloxy or heteroaryloxy group containing 5 to 20 ring atoms, an arylamine or heteroarylamine group containing 5 to 20 ring atoms, or any combination thereof, where any two adjacent substituents of R¹⁰⁵-R¹⁰⁸ may form a monocyclic or polycyclic aliphatic or aromatic ring system with each other and/or with the rings bonded thereto.

In some embodiments, at least two of R¹⁰¹-R¹⁰⁴ are each independently selected from one of formulas (Ia-1)-(Ia-4).

In some embodiments, at least three of R¹⁰¹-R¹⁰⁴ are each independently selected from one of formulas (Ia-1)-(Ia-4).

In some embodiments, R¹⁰¹-R¹⁰⁴ are each independently selected from one of formulas (Ia-1)-(Ia-4).

In some embodiments, among the R¹⁰¹-R¹⁰⁴ as described herein, R¹⁰¹ and R¹⁰³, or R¹⁰² and R¹⁰⁴ are the same structural unit.

In some embodiments, R¹⁰¹-R¹⁰⁴ are the same structural unit.

In some embodiments, the emitter E is disclosed in International Application Publication No. WO2022213993A1, which is hereby incorporated by reference in its entirety.

In some embodiments, the FWHM of the emission spectrum of the emitter E≤50 nm, preferably ≤40 nm, more preferably ≤35 nm, and most preferably ≤30 nm.

In some embodiments, the photoluminescence quantum yield (PLQY) of the emitter E≥50%, preferably ≥60%, more preferably ≥70%, and most preferably ≥80%.

In some embodiments, the emitter E comprises a structural unit of formula (1), formula (2), formula (3), or formula (4):

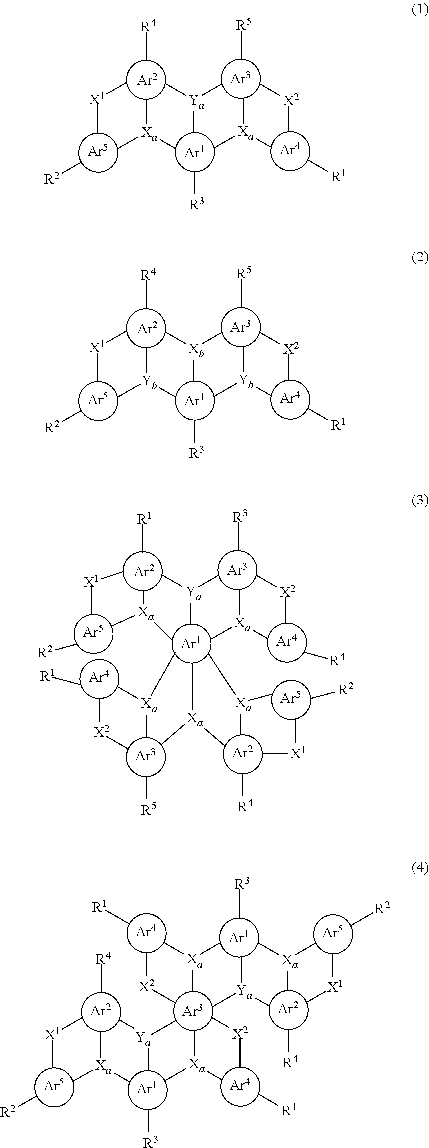

Where each of Ar¹ to Ar³ is independently selected from an aromatic group or a heteroaromatic group containing 5 to 24 ring atoms; each of Ar⁴ and Ar⁵ is independently selected from null, an aromatic group or a heteroaromatic group containing 5 to 24 ring atoms; when neither Ar⁴ nor Ar⁵ is null, each of Xₐ and X_{b} is independently selected from N, C(R⁶), or Si(R⁶); each of Yₐ and Y_{b} is independently selected from B, P═O, C(R⁶), or Si(R⁶); when Ar⁴ and/or Ar⁵ is null, each X_{b} is independently selected from N, C(R⁶), or Si(R⁶); each Yₐ is independently selected from B, P═O, C(R⁶), or Si(R⁶); each of Xₐ and Y_{b} is independently selected from N(R⁶), C(R⁶R⁷), Si(R⁶R⁷), C═O, O, C═N(R⁶), C═C(R⁶R⁷), P(R⁶), P(═O)R⁶, S, S═O, or SO₂; each of X¹ and X² is independently null or a bridging group;
each of R¹ to R⁷ at each occurrence is independently selected from —H, -D, a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents or any combination thereof, where one or more R¹-R⁷ may form a monocyclic or polycyclic aliphatic or aromatic ring system with each other and/or with the rings bonded thereto.

In some embodiments, each of R¹ to R⁷ at each occurrence is independently selected from —H, -D, a C₁-C₁₀ linear alkyl group, a C₁-C₁₀ linear haloalkyl group, a C₁-C₁₀ linear alkoxy group, a C₁-C₁₀ linear thioalkoxy group, a C₃-C₁₀ branched/cyclic alkyl group, a C₃-C₁₀ branched/cyclic haloalkyl group, a C₃-C₁₀ branched/cyclic alkoxy group, a C₃-C₁₀ branched/cyclic thioalkoxy group, a C₃-C₁₀ branched/cyclic silyl group, a C₁-C₁₀ ketone group, a C₂-C₁₀ alkoxycarbonyl group, a C₆-C₁₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 20 ring atoms, an aryloxy or heteroaryloxy group containing 5 to 20 ring atoms, an arylamine or heteroarylamine group containing 5 to 20 ring atoms, a disubstituted unit in any position of the above substituents or any combination thereof, where one or more R¹-R⁷ may form a monocyclic or polycyclic aliphatic or aromatic ring system with each other and/or with the rings bonded thereto.

In some embodiments, the emitter E comprises a structural unit of formula (Ia), formula (2a), formula (3a), or formula (4a):

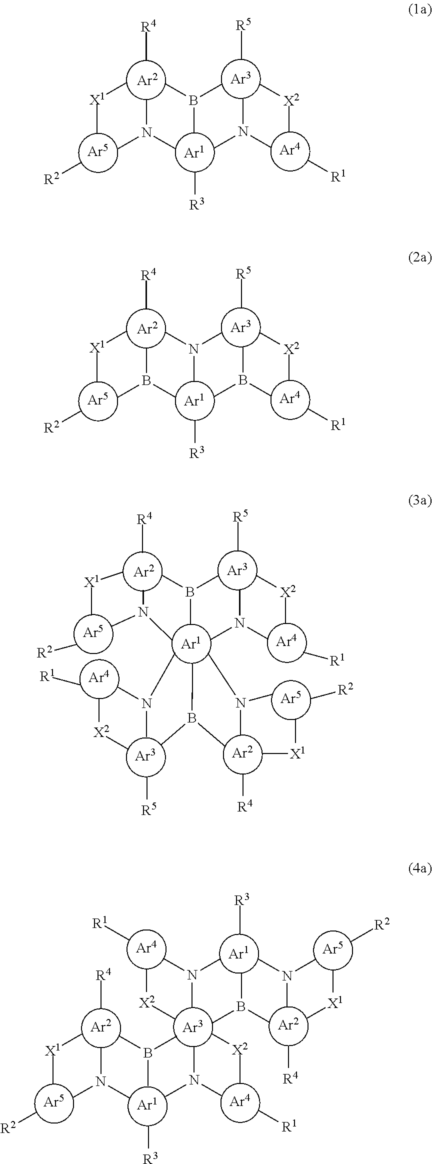

where Ar¹—Ar⁵, X¹, X², and R¹-R⁵ are identically defined as described herein.

In some embodiments, each of X¹ and X² is independently O or S; in some embodiments, each of X¹ and X² is O.

In some embodiments, at least one of X¹ or X² is null; particularly preferably, both are null, in which case the emitter E comprises a structural unit of formula (Ib), formula (2b), formula (3b), or formula (4b):

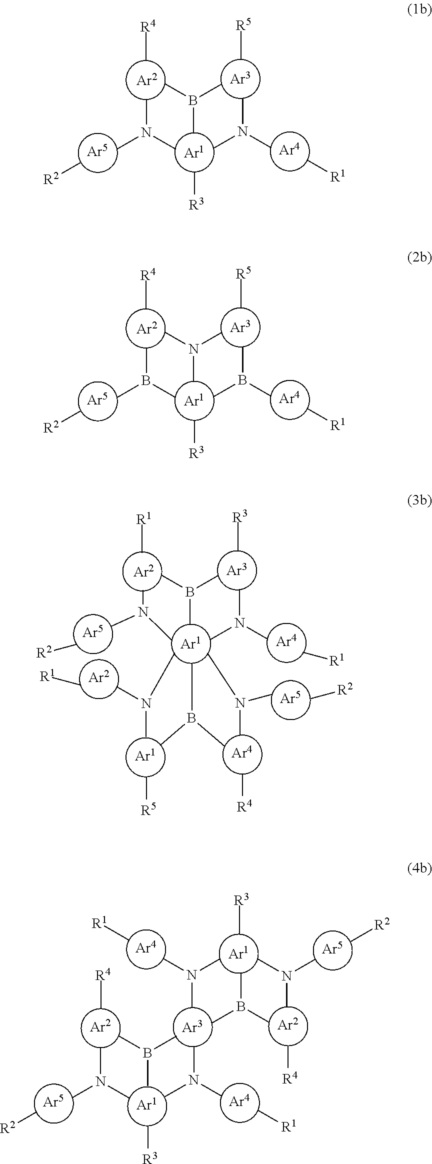

Where Ar¹—Ar⁵ and R¹-R⁵ are identically defined as described herein.

In some embodiments, at least one of X¹ or X² is a single bond; particularly preferably, both are single bonds, and the emitter E comprises a structural unit of formula (Ic), formula (2c), formula (3c), or formula (4c):

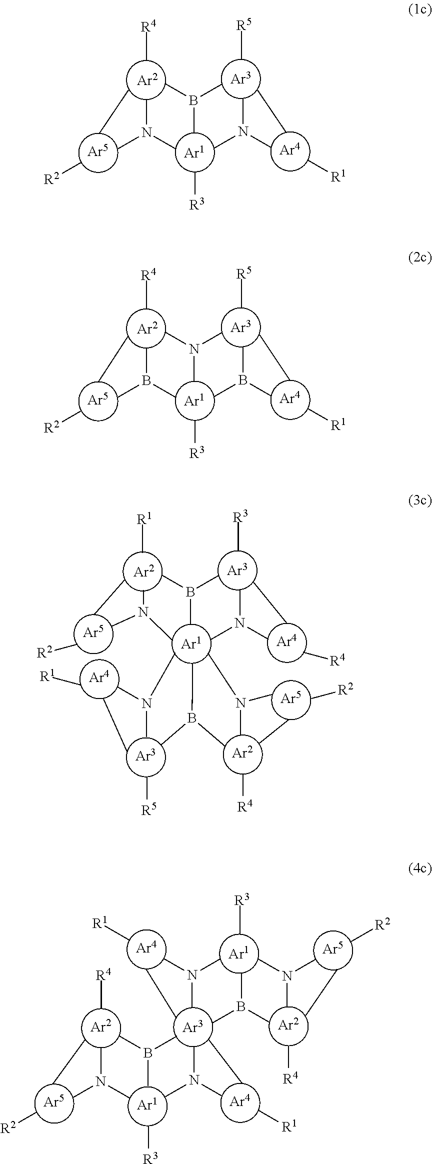

Where Ar¹—Ar⁵ and R¹-R⁵ are identically defined as described herein.

In some embodiments, X¹, X² at each occurrence are the same or different di-bridging group, the preferred di-bridging groups are the following formulas:

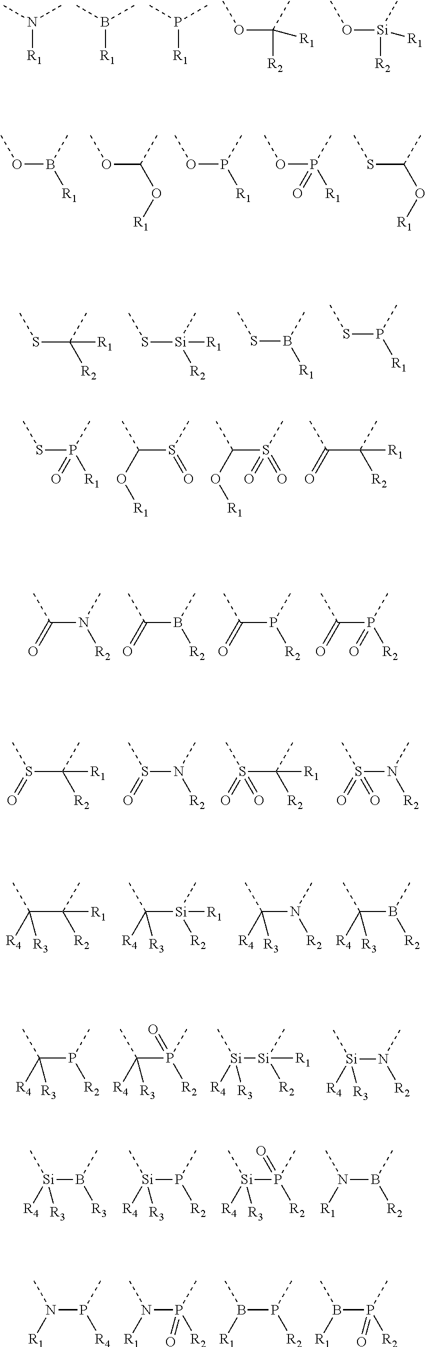

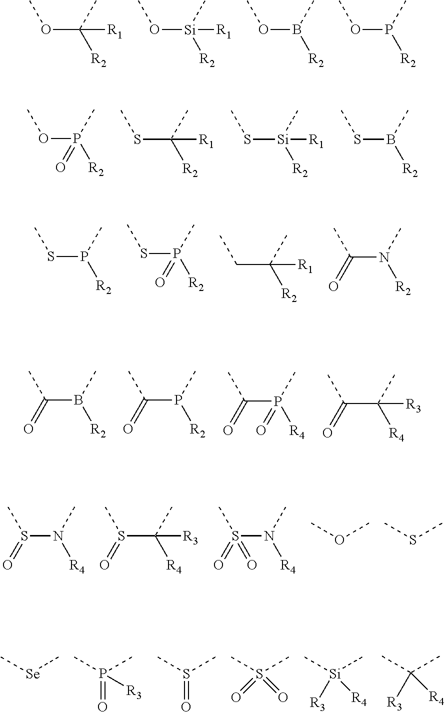

Where R1, R2, R3, and R4 are identically defined as the above-mentioned R1, and the dashed bonds refer to the covalent bonds connecting to the adjacent structural units.

For the purposes of the present disclosure, the aromatic ring system contains 6 to 20 carbon atoms in the ring system, the heteroaromatic ring system contains 1 to 20 carbon atoms and at least one heteroatom in the ring system, provided that the total number of carbon atoms and heteroatoms is at least 5. The heteroatoms are preferably selected from Si, N, P, O, S and/or Ge, particularly preferably selected from Si, N, P, O and/or S. For the purposes of the present disclosure, the aromatic or heteroaromatic ring systems contain not only aromatic or heteroaromatic groups, but also have a plurality of aryl or heteroaryl groups linked by short non-aromatic units (<10% of non-H atoms, preferably <5% of non-H atoms, such as C, N or O atoms). Therefore, a system such as 9,9′-spirobifluorene, 9,9-diarylfluorene, triarylamine, diaryl ether, and the like is also considered to be aromatic ring systems for the purposes of this disclosure.

For the purposes of the present invention, any H atom of the organic compound H or the emitter E may be substituted with R¹⁰. R¹⁰ is defined as the above-mentioned R¹⁰⁵, which may be preferably selected from: (1) a C₁-C₁₀ alkyl group, particularly preferably selected from the following groups: methyl, ethyl, n-propyl, isopropyl, cyclopropyl, n-butyl, isobutyl, sec-butyl, t-butyl, cyclobutyl, 2-methylbutyl, n-pentyl, n-hexyl, cyclohexyl, n-heptyl, cycloheptyl, n-octyl, cyclooctyl, 2-methylheptyl, trifluoromethyl, pentafluoroethyl, 2,2,2-trifluoroethyl, vinyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, or octenyl; (2) a C₁-C₁₀ alkoxy group, particularly preferably methoxy, ethoxy, n-propoxy, isopropoxy, n-butoxy, isobutoxy, sec-butoxy, tert-butoxy, or 2-methylbutoxy; (3) a C₂-C₁₀ aryl or heteroaryl group, which may be monovalent or divalent depending on the application, and in each case can also be optionally substituted with R¹⁰ and may be attached to an aromatic or heteroaromatic ring at any desired position, particularly preferably selected from the following: benzene, naphthalene, anthracene, dihydropyrene, chrysene, pyrene, fluoranthene, naphthacene, pentacene, benzopyrene, furan, benzofuran, isobenzofuran, dibenzofuran, thiophene, benzothiophene, isobenzothiophene, thiofluorene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenimidazole, pyridimidazole, pyrazine-imidazole, quinoxaline-imidazole, oxazole, benzoxazole, naphthoxazole, anthracenazole, phenoxazole, isoxazole, 1,2-thiazole, 1,3-thiazole, benzothiazole, pyridazine, benzopyridazine, pyrimidine, benzopyrimidine, quinoxaline, pyrazine, phenazine, 1,5-naphthyridine, azocarbazole, benzocholine, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thinadiazole, 1,2,4-thiadiazole, 1,2,5-thiadiazole, 1,3,4-thiadiazole, 1,3,5-triazine, 1,2,4-triazine, 1,2,3-triazine, tetrazole. 1,2,4,5-tetrazine, 1,2,3,4-tetrazine, 1,2,3,5-tetrazine, purine, pteridine, indolizine, or benzothiadiazole. For the purposes of the present disclosure, aromatic and heteroaromatic ring systems are particularly considered to be, in addition to the above-mentioned aryl and heteroaryl groups, also refer to biphenylene, terphenylene, fluorene, spirobifluorene, dihydrophenanthrene, tetrahydropyrene, cis-indenofluorene, or trans-indenofluorene.

In some embodiments, Ar¹, Ar², and Ar¹—Ar⁵ of the organic compound H and the emitter E are the same or different and are each independently selected from the group consisting of aromatic or heteroaromatic groups with 5 to 20 ring atoms; preferably 5 to 18 ring atoms, more preferably 5 to 15 ring atoms; and most preferably 5 to 10 ring atoms; they may be unsubstituted or substituted with one or two R¹⁰. Preferred aromatic or heteraromatic groups include benzene, naphthalene, anthracene, phenanthrene, pyridine, benzofuran, pyrene, or thiophene.

In some embodiments, Ar¹, Ar², and Ar¹—Ar⁵ are each independently selected from the following structural formulas:

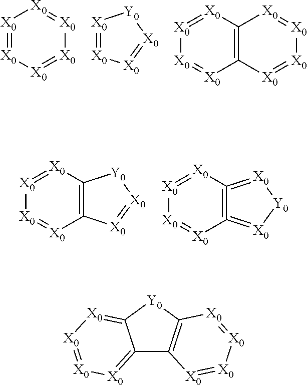

Where each X₀ is independently CR¹¹ or N; each Y₀ is independently selected from NR¹¹, CR¹²R¹³, SiR¹⁴R¹⁵, C(═O), S, or O; R^{D}, R¹², R¹³, R¹⁴, and R¹⁵ are identically defined as the above-mentioned R¹⁰¹.

Further, Ar¹, Ar², Ar¹—Ar⁵ are each independently selected from one of the following structural formulas or any combination thereof, which can be further arbitrarily substituted:

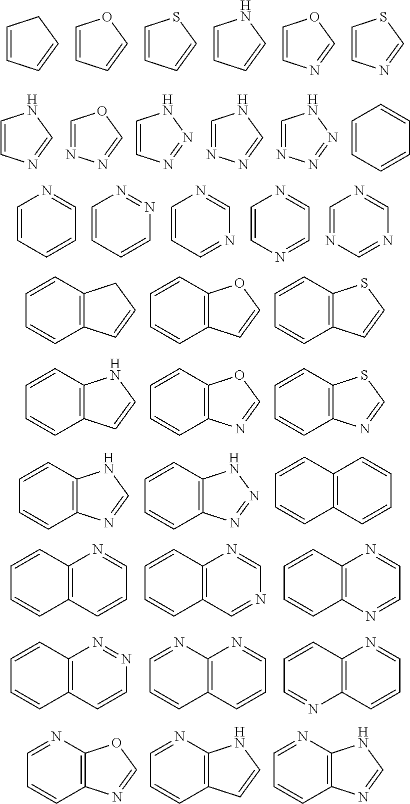

In some embodiments, each of Ar¹—Ar² and Ar¹—Ar⁵ is a phenyl group.

In some embodiments, at least one of Ar⁴ and Ar⁵ is null; particularly preferably both are null, in which case the emitter E comprises a structural unit of formula (1d), formula (2d), formula (1e), formula (2e), formula (3d), formula (4di), or formula (4d₂):

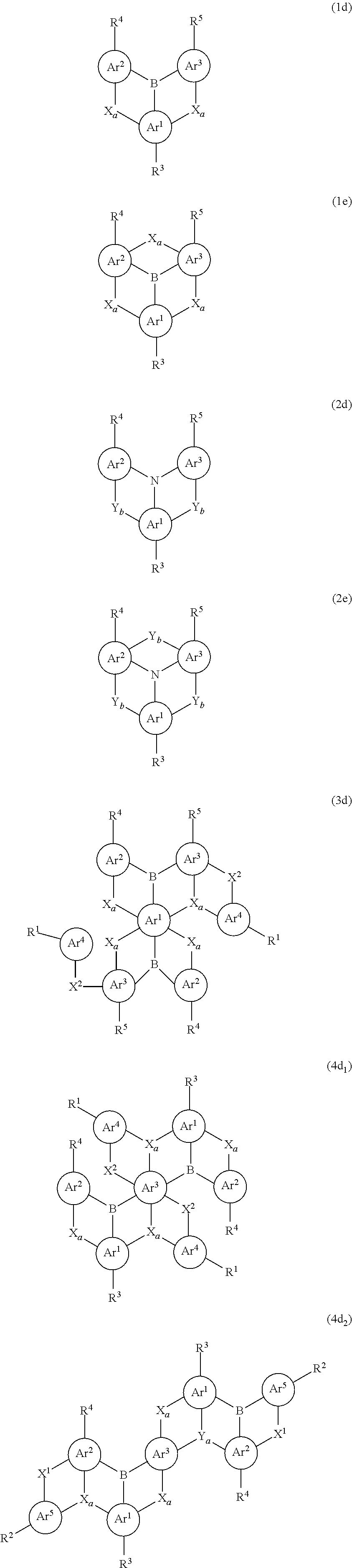

Where Ar¹—Ar³, Ar⁴—Ar⁵, X¹, X², Xₐ, Yₐ, Y_{b}, and R¹-R⁵ are identically defined as described herein.

Preferably, each Xₐ in the formulas (1d) and (1e) is independently selected from N(R⁶), C(R⁶R⁷), Si(R⁶R⁷), O, or S.

Preferably, each Y_{b} in the formulas (2d) and (2e) is independently selected from C═O, O, S, P(═O)R⁶, S═O, or SO₂; and particularly preferably from C═O.

Preferably, each Xₐ in the formulas (3d), (4di), and (4d₂) is independently selected from N(R⁶), C(R⁶R⁷), Si(R⁶R⁷), O, or S.

In some embodiments, the emitter E comprises a structural unit of formulas (1f)-(1i):

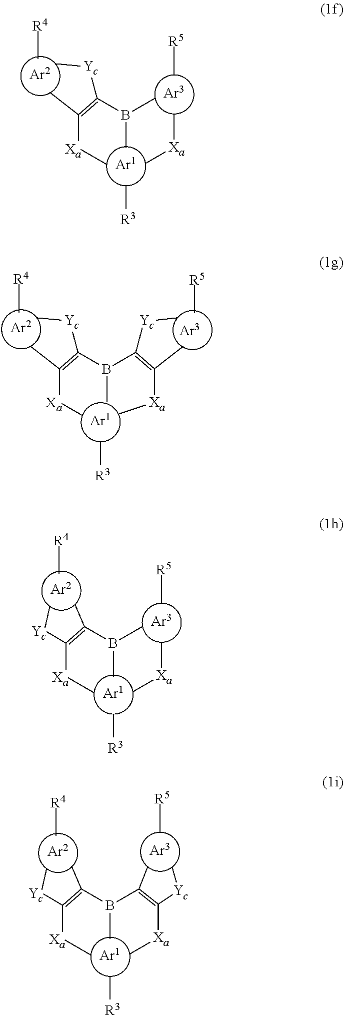

Where each Y, is independently O or S; Ar¹—Ar³, Xₐ, and R³-R⁵ are identically defined as described herein.

In some embodiments, each of Ar² and Ar³ is preferably selected from the following structural units, which can be further arbitrarily substituted:

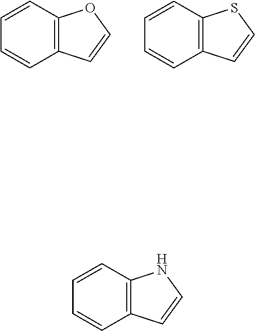

In some embodiments, in the structural units of formulas (1)-(1i), (2)-(2e), (3)-(3d), (4)-(4d₂), R¹-R⁵, in multiple occurrences, are each independently selected from the following structural units or any combination thereof:

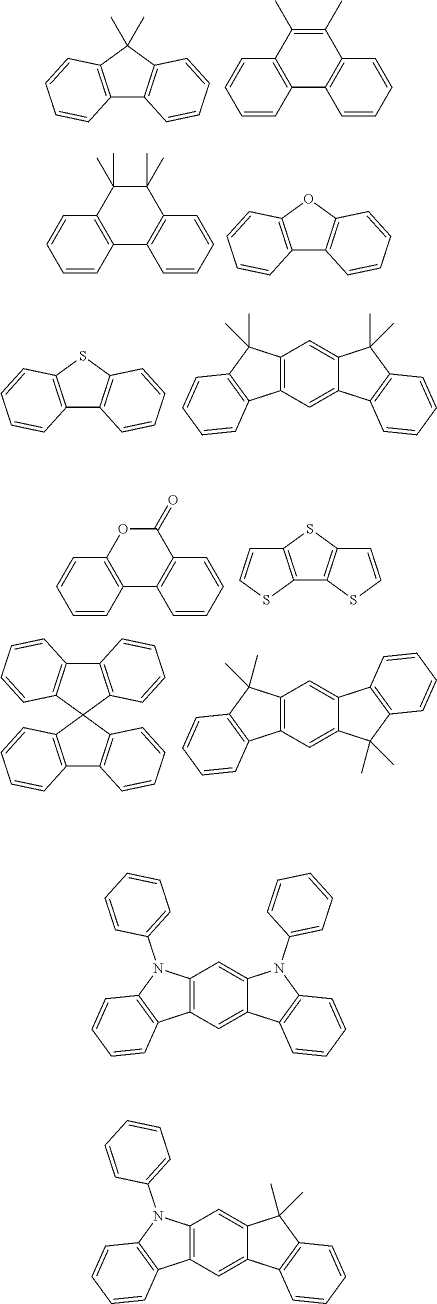

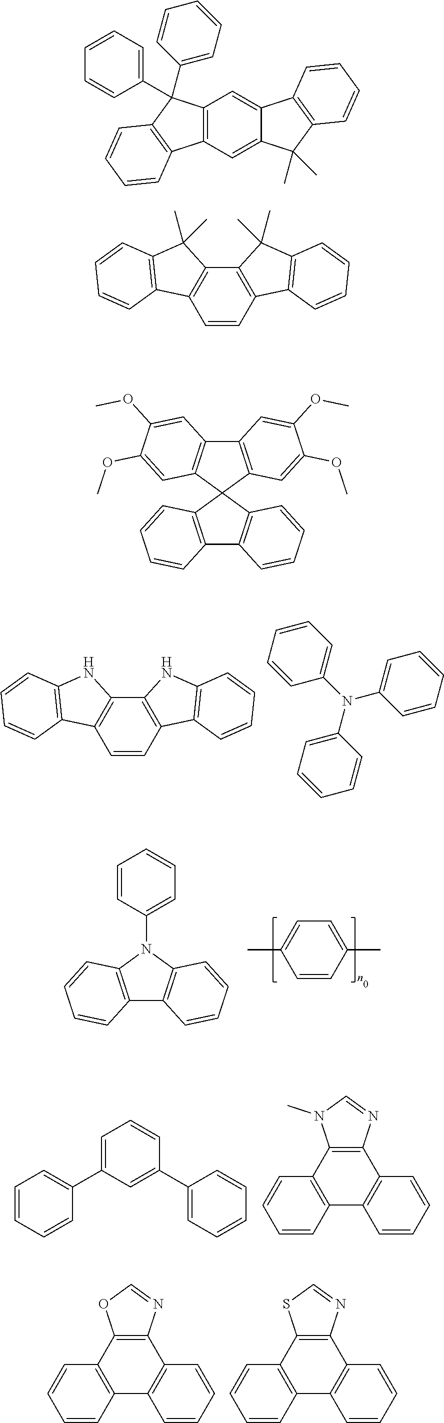

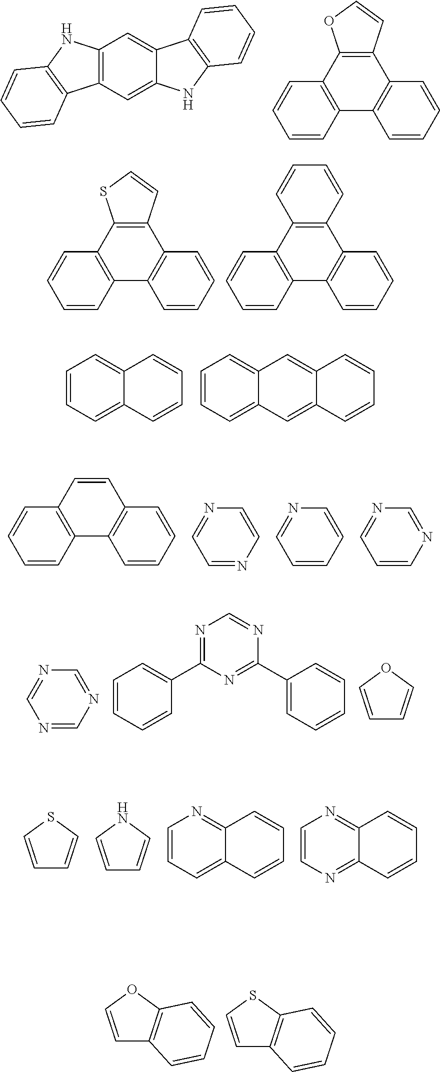

Where n₀ is an integer from 1 to 4.

In some embodiments, the emitter E comprises the structure as shown below:

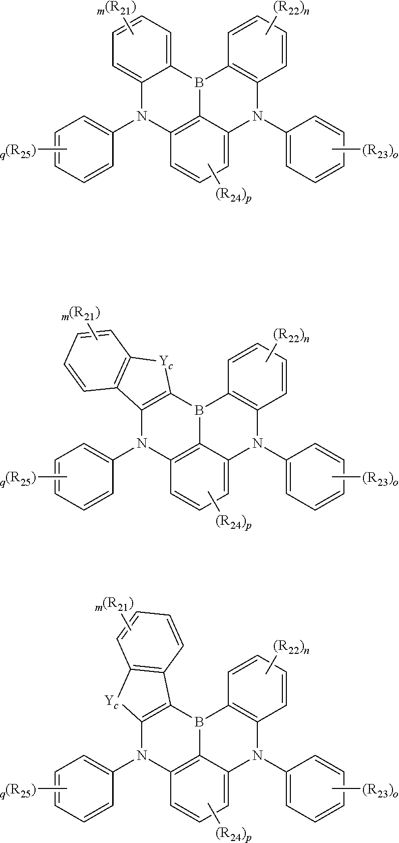

Where each Y, is identically defined as described herein; each of R₂₁ to R₂₅ is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, or any combination thereof, where one or more R₂₁-R₂₅ may form a monocyclic or polycyclic aliphatic or aromatic ring system with each other and/or with the rings bonded thereto; and at least one of R₂₁-R₂₅ comprises an alcohol-soluble or water-soluble group; m and n are integers from 0 to 4; o and q are integers from 0 to 5; p is an integer from 0 to 3.

Preferably, each of R₂₁ to R₂₅ is independently selected from —H, -D, a C₁-C₁₀ linear alkyl group, a C₁-C₁₀ linear alkoxy group, a C₁-C₁₀ linear thioalkoxy group, a C₃-C₁₀ branched/cyclic alkyl group, a C₃-C₁₀ branched/cyclic alkoxy group, a C₃-C₁₀ branched/cyclic thioalkoxy group, a C₃-C₁₀ branched/cyclic silyl group, a C₁-C₁₀ ketone group, a C₂-C₁₀ alkoxycarbonyl group, a C₆-C₁₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 20 ring atoms, an aryloxy or heteroaryloxy group containing 5 to 20 ring atoms, or any combination thereof, where one or more R₂₁-R₂₅ may form a monocyclic or polycyclic aliphatic or aromatic ring system with each other and/or with the rings bonded thereto.

In embodiments of the present disclosure, the energy level structure of the organic materials, triplet energy level (T1), singlet energy level (Si), highest occupied molecular orbital (HOMO), lowest unoccupied molecular orbital (LUMO), and oscillator strength f play key roles. The determination of these energy levels is introduced as follows.

HOMO and LUMO energy levels can be measured by optoelectronic effect, for example, by XPS (X-ray photoelectron spectroscopy), UPS (UV photoelectron spectroscopy), or by cyclic voltammetry (hereinafter referred to as CV). Recently, quantum chemical methods, such as density functional theory (hereinafter referred to as DFT), are becoming effective methods for calculating the molecular orbital energy levels.

The triplet energy level (T1) of the organic materials can be measured by low-temperature time-resolved spectroscopy, or calculated by quantum simulation (for example, by time-dependent DFT), for instance with the commercial software Gaussian 09W (Gaussian Inc.), the specific simulation method is as follows. The singlet energy level Si of the organic materials can be determined by the absorption spectrum or the emission spectrum, and can also be calculated by quantum simulation (such as Time-dependent DFT); the oscillator strength f can also be calculated by quantum simulation (such as Time-dependent DFT).

It should be noted that the absolute values of HOMO, LUMO, T1 and Si may depend on the measurement method or calculation method used. Even for the same method, different ways of evaluation, for example, using either the onset or peak value of a CV curve as reference, may result in different HOMO/LUMO values. Therefore, reasonable and meaningful comparison should be carried out by employing the same measurement and evaluation methods. In the embodiments of the present disclosure, the values of HOMO, LUMO, T1 and Si are based on the time-dependent DFT simulation, which however should not exclude the applications of other measurement or calculation methods.

In some embodiments, the (S1-T1) of the emitter E≤0.30 eV, preferably ≤0.25 eV, more preferably ≤0.20 eV, further preferably ≤0.15 eV, and most preferably ≤0.10 eV.

In some embodiments, the emitter E of the formulation is a small molecule or a polymer.

In some embodiments, the emitter E has good solubility in the resin or resin prepolymer.

In some embodiments, the organic compound H has good solubility in the resin or resin prepolymer.

In some embodiments, the organic compound H and/or the emitter E comprises at least one alcohol-soluble or water-soluble group, as disclosed in International Application Publication No. WO2022078434A1, which is hereby incorporated by reference in its entirety.

In some embodiments, the organic compound H and/or the emitter E comprises at least two alcohol-soluble or water-soluble groups.

In some embodiments, the organic compound H and/or the emitter E comprises at least three alcohol-soluble or water-soluble groups.

In some embodiments, the alcohol-soluble or water-soluble group of the organic compound H and/or the emitter E is selected from: alcohols, aldehydes, acids, crown ethers, polyethers, or primary amines.

Preferably, the alcohol-soluble or water-soluble group is selected from the following structures:

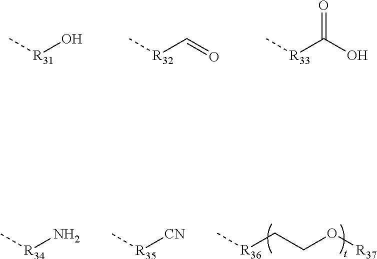

Where each of R₃₁ to R₃₇ is independently selected from a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F a cross-linkable group, a substituted/unsubstituted aromatic or heteroaromatic group containing 5 to 40 ring atoms, an aryloxy or heteroaryloxy group containing 5 to 40 ring atoms, or any combination thereof, where one or more R₃₁-R₃₇ may form a monocyclic or polycyclic aliphatic or aromatic ring system with each other and/or with the rings bonded thereto; t is an integer >0.

Furthermore, the individual H atoms or CH₂ groups as described herein may be substituted with the above-mentioned groups or R₀. R₀ is selected from C₁-C₄₀ alkyl groups, preferably selected from the group consisting of methyl, ethyl, n-propyl, isopropyl, cyclopropyl, n-butyl, isobutyl, sec-butyl, t-butyl, cyclobutyl, methylbutyl, n-pentyl, sec-pentyl, cyclopentyl, n-hexyl, cyclohexyl, n-heptyl, cycloheptyl, n-octyl, cyclooctyl, ethylhexyl, trifluoromethyl, pentafluoroethyl, trifluoroethyl, vinyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, and octynyl; C₁-C₄₀ alkoxy groups, such as methoxy, trifluoromethoxy, ethoxy, n-propoxy, isopropoxy, n-butoxy, isobutoxy, sec-butoxy, tert-butoxy, or methylbutoxy.

Examples of the emitters E which can be further arbitrarily substituted are listed below, but not limited to:

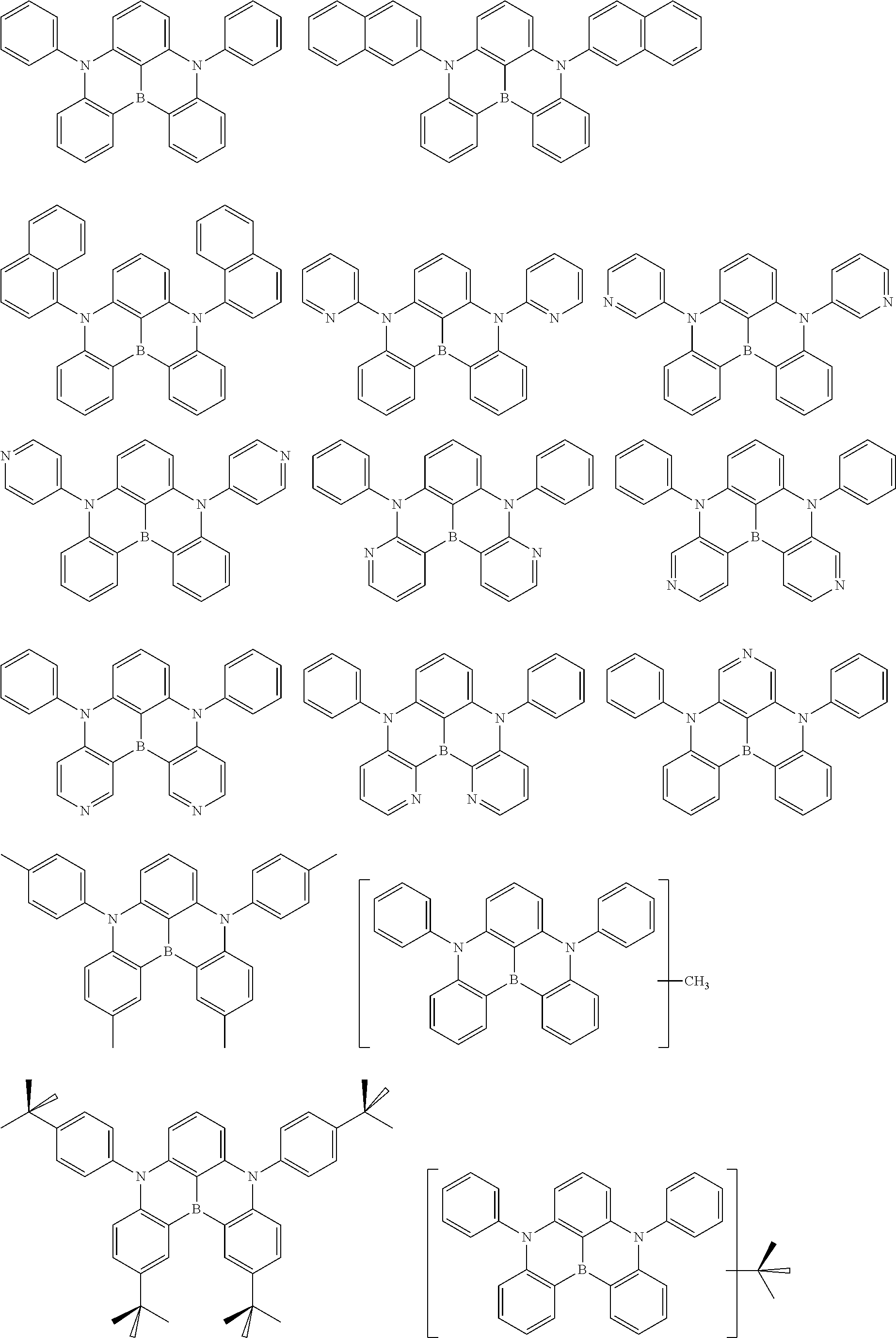

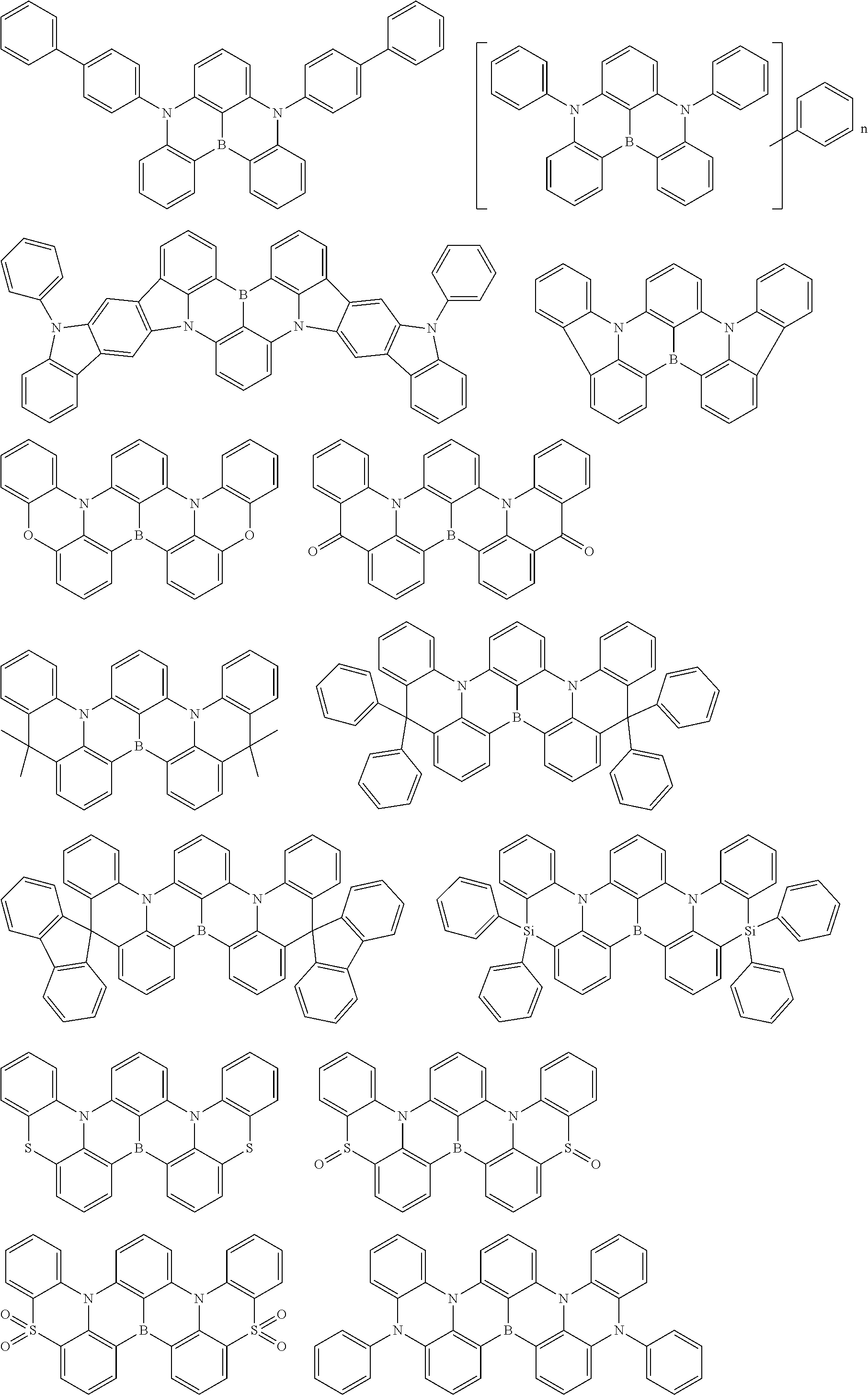

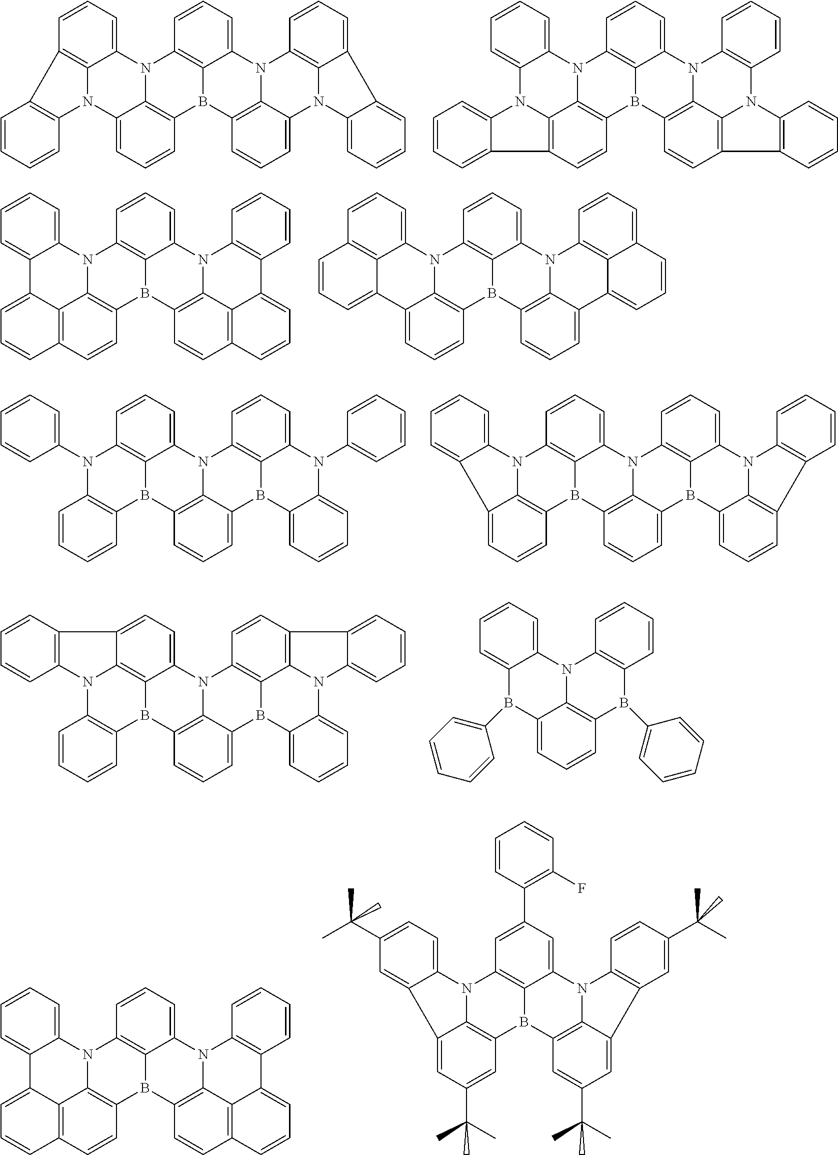

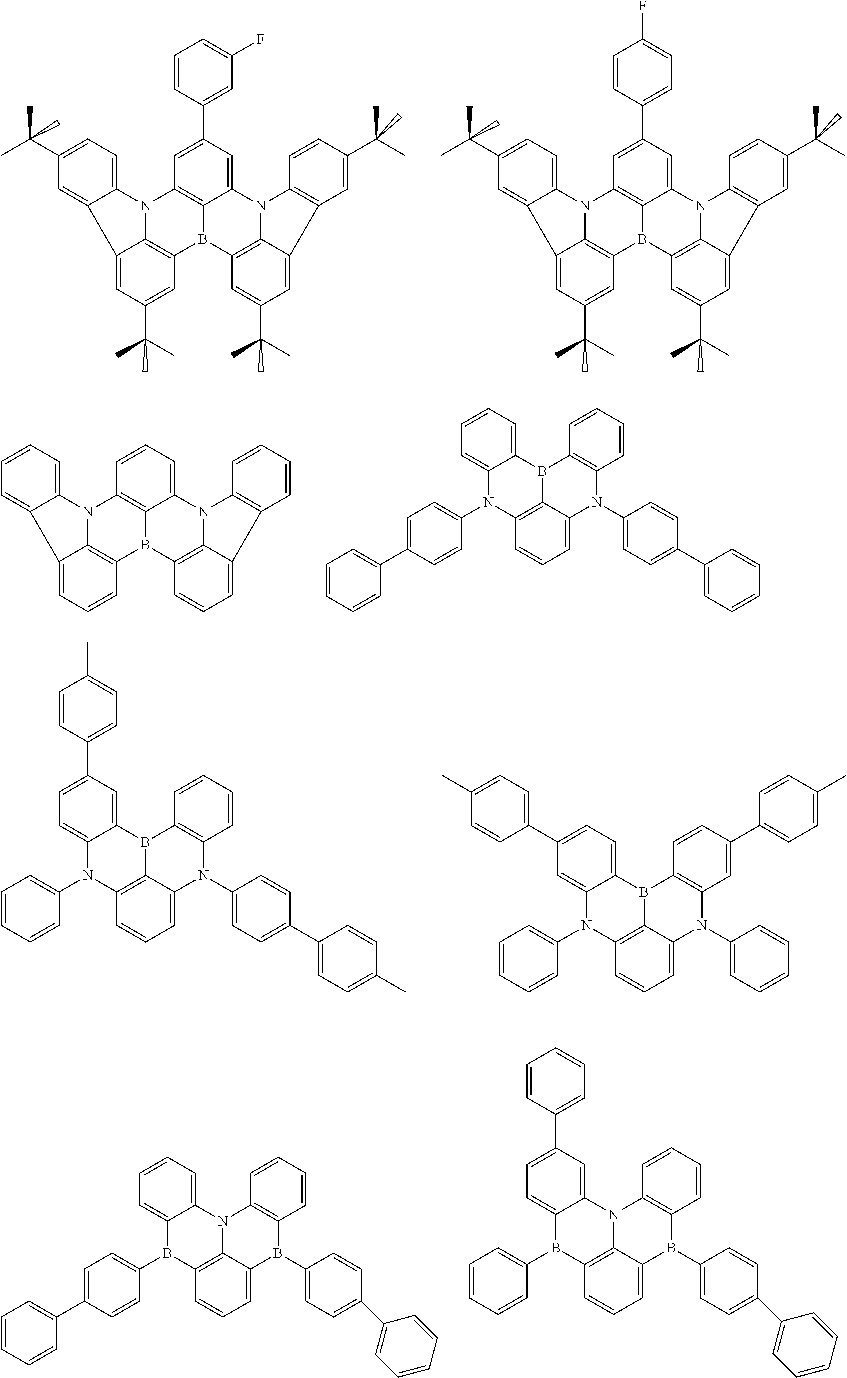

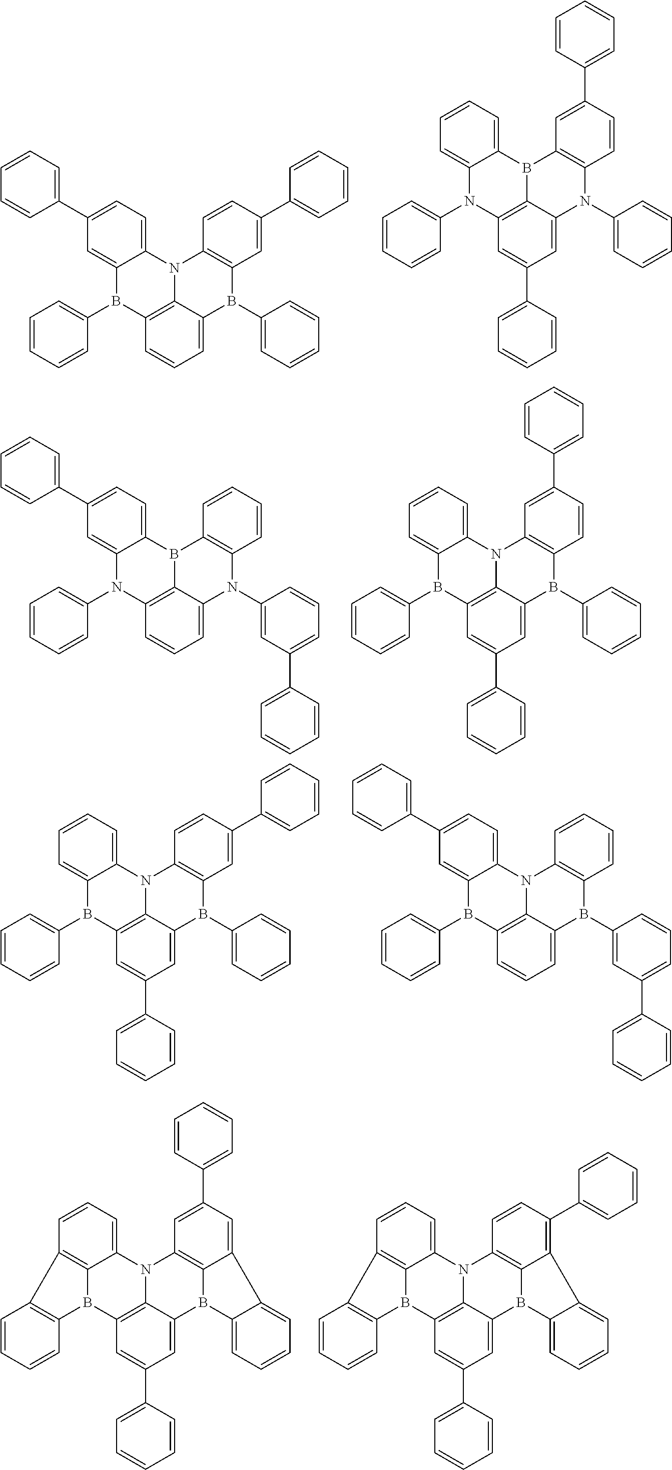

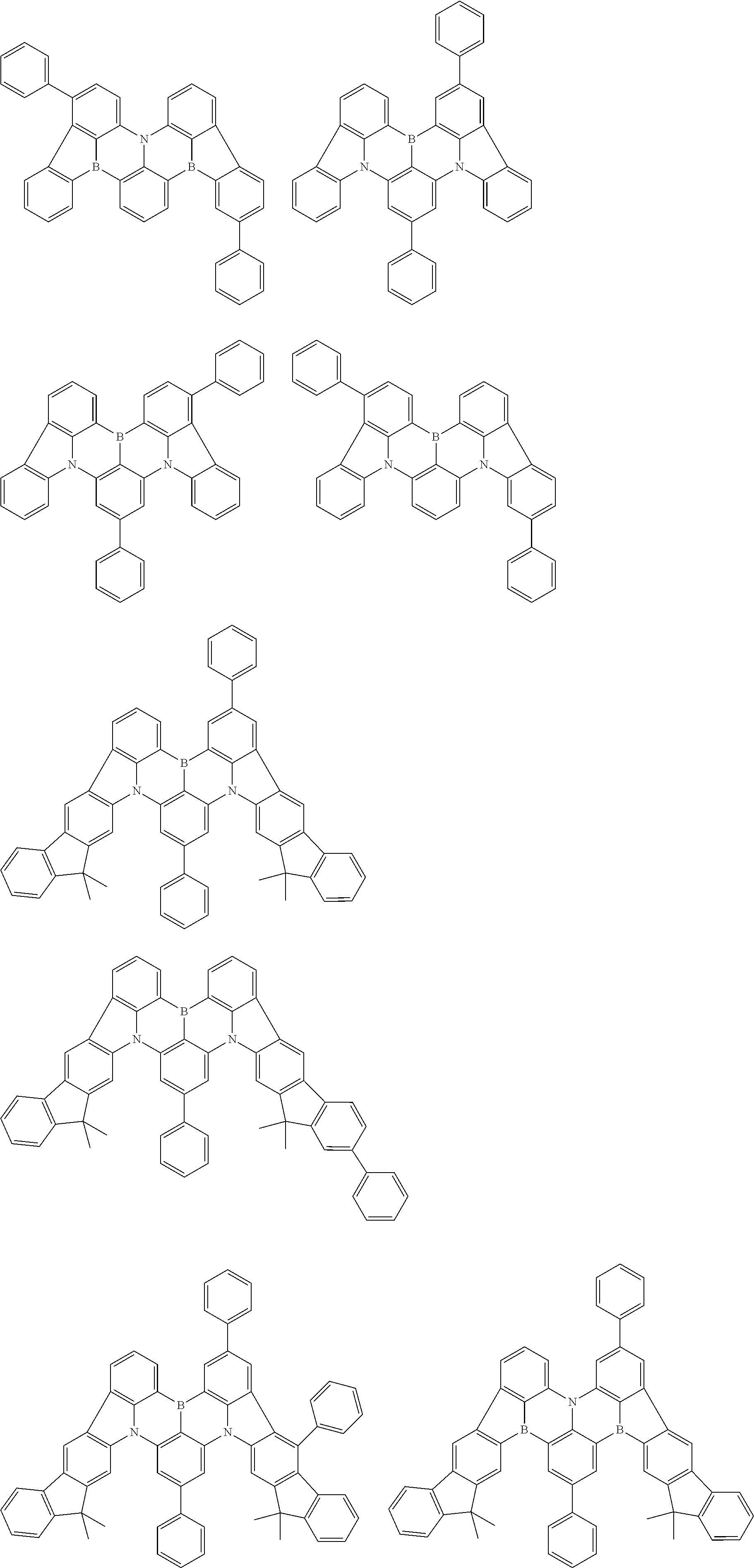

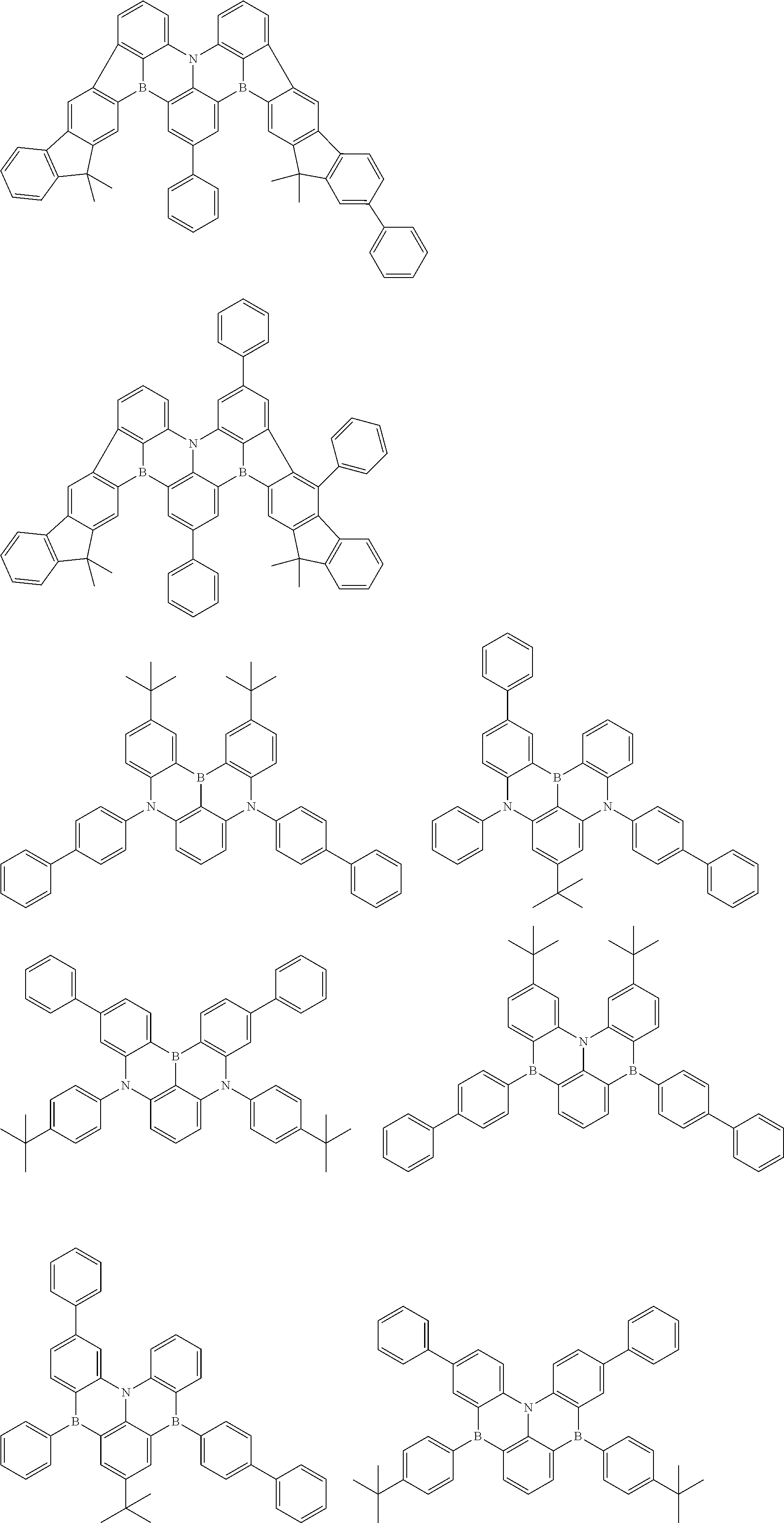

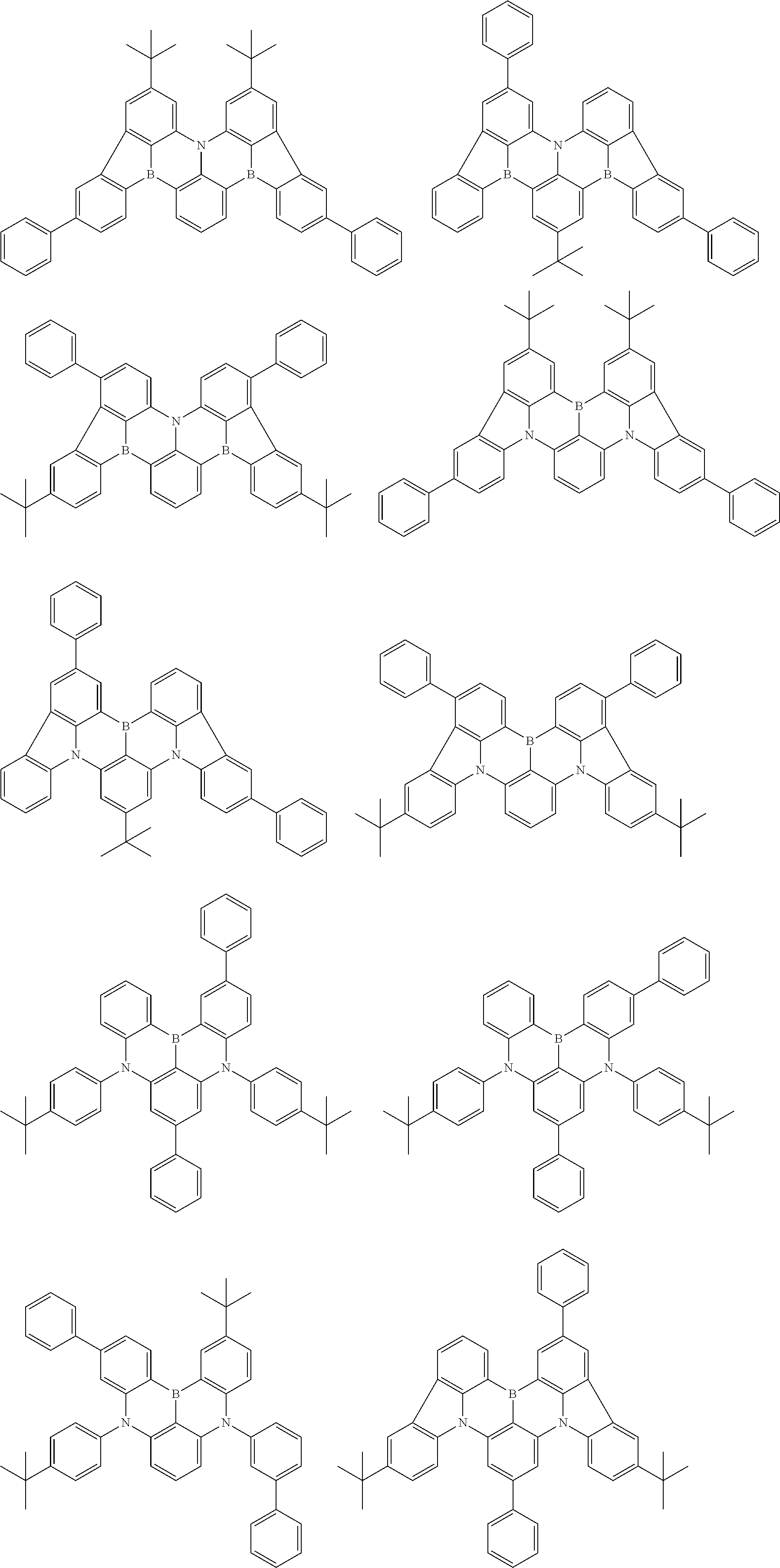

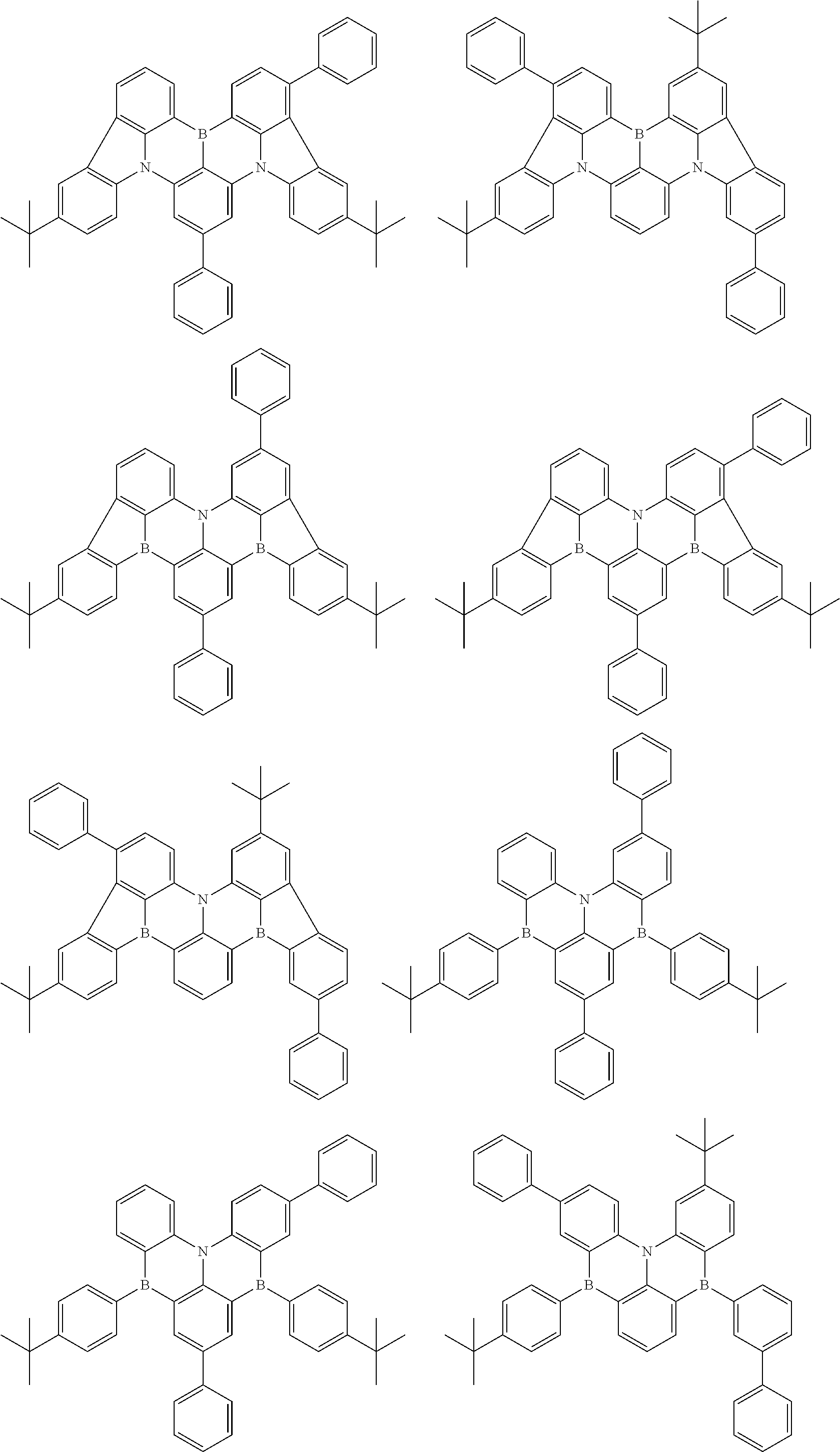

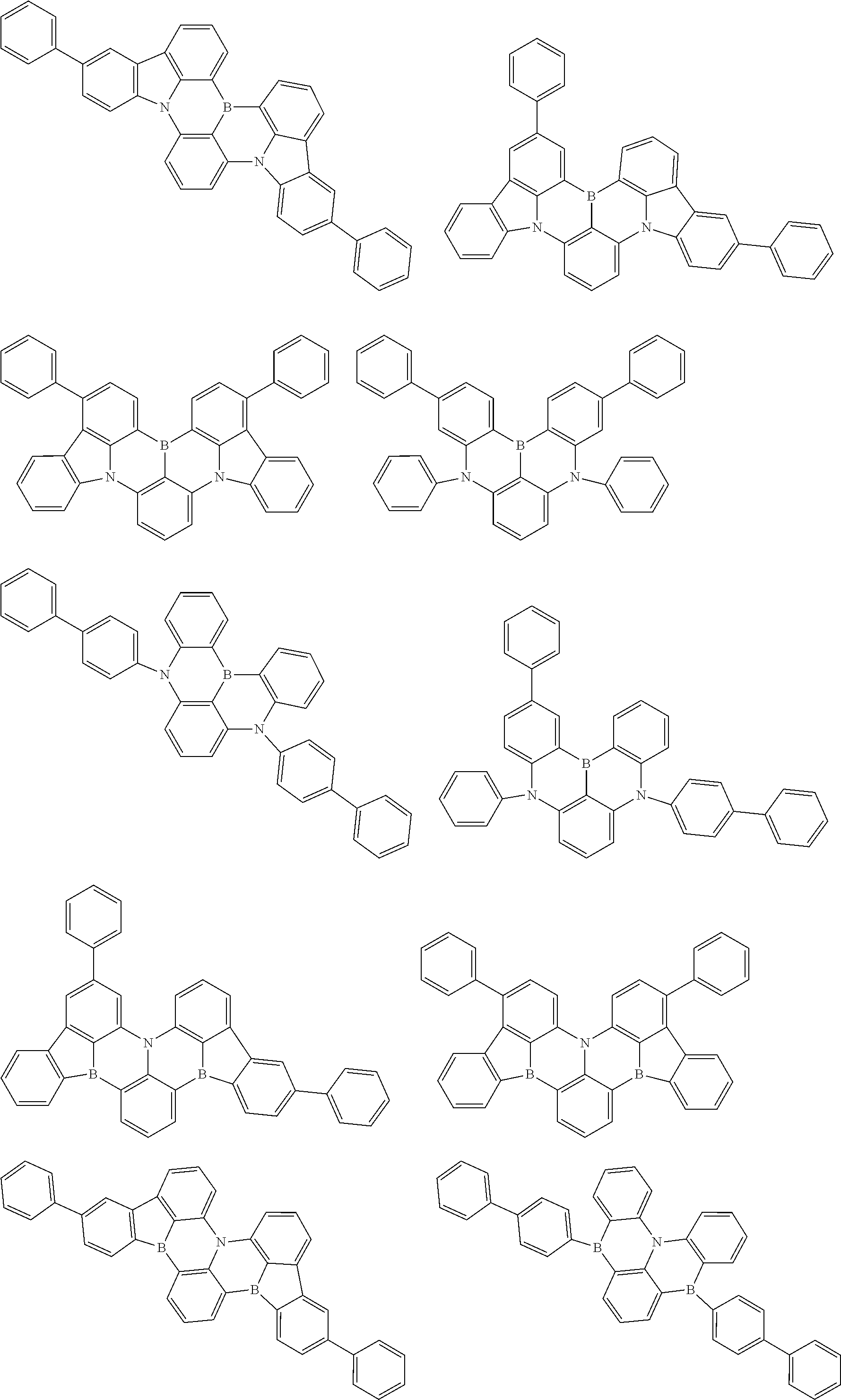

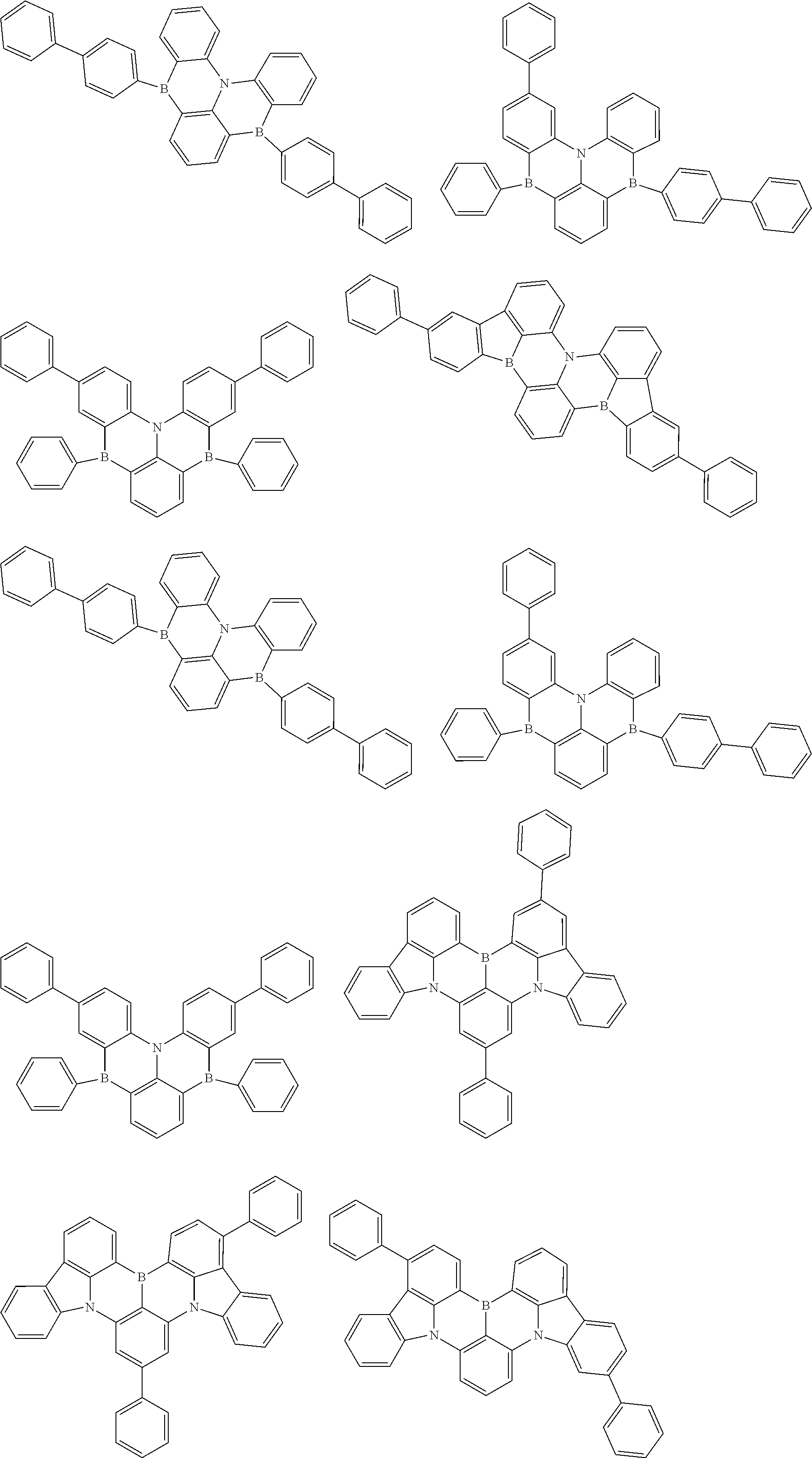

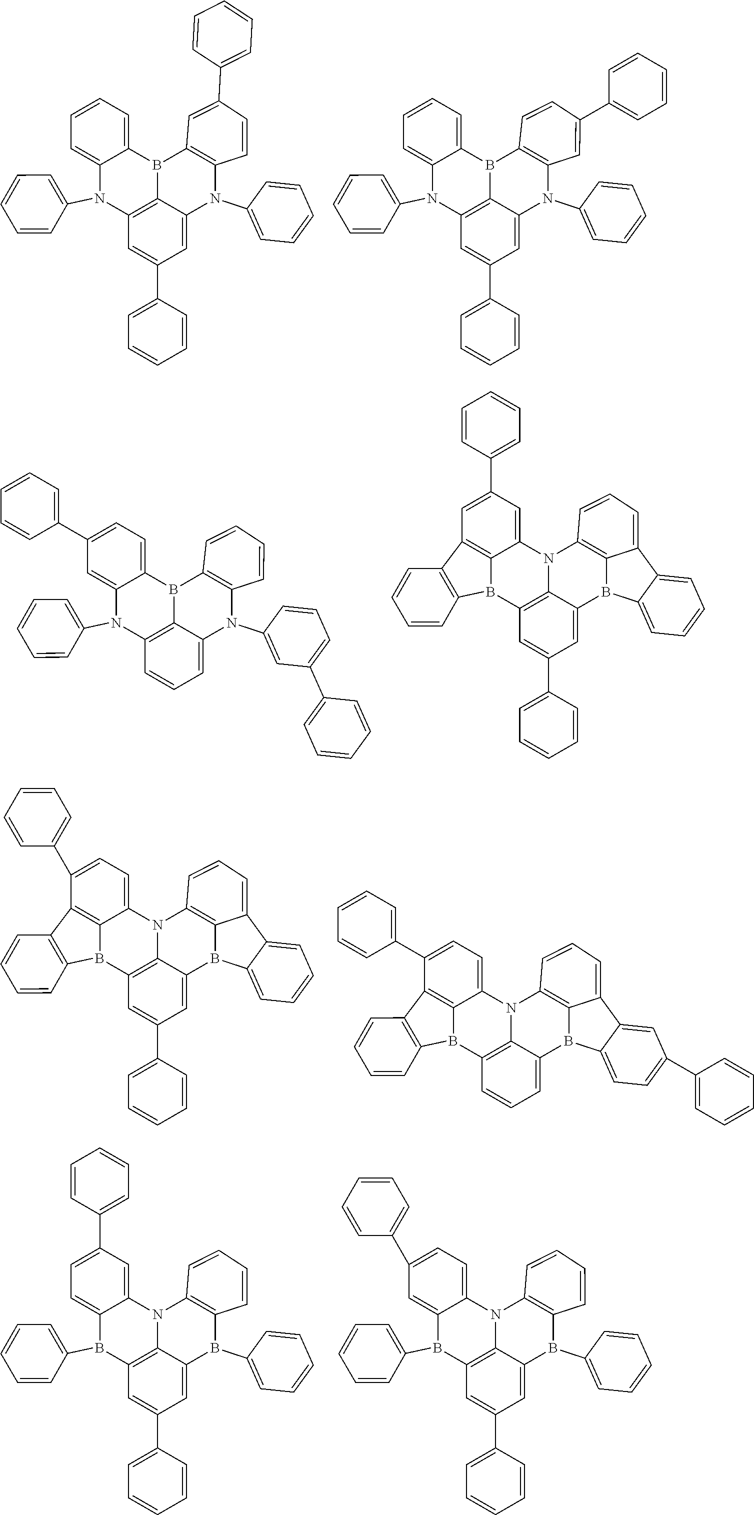

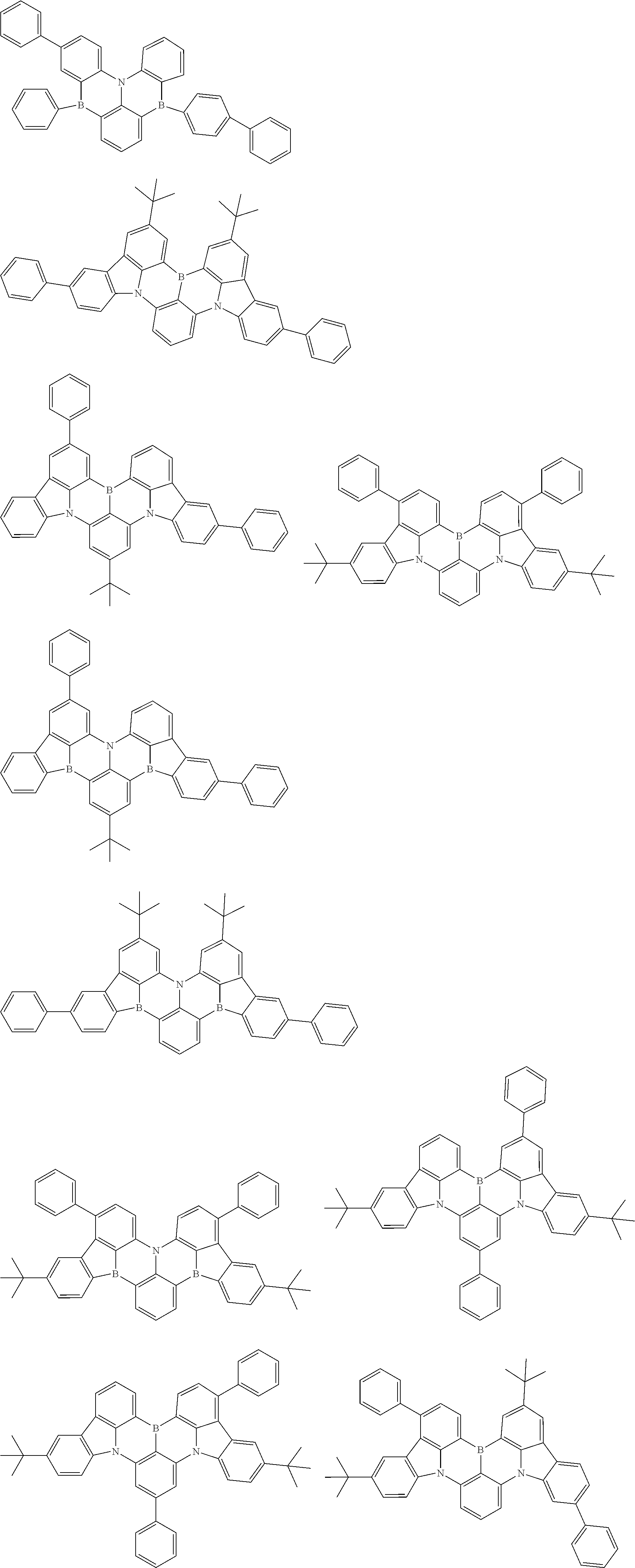

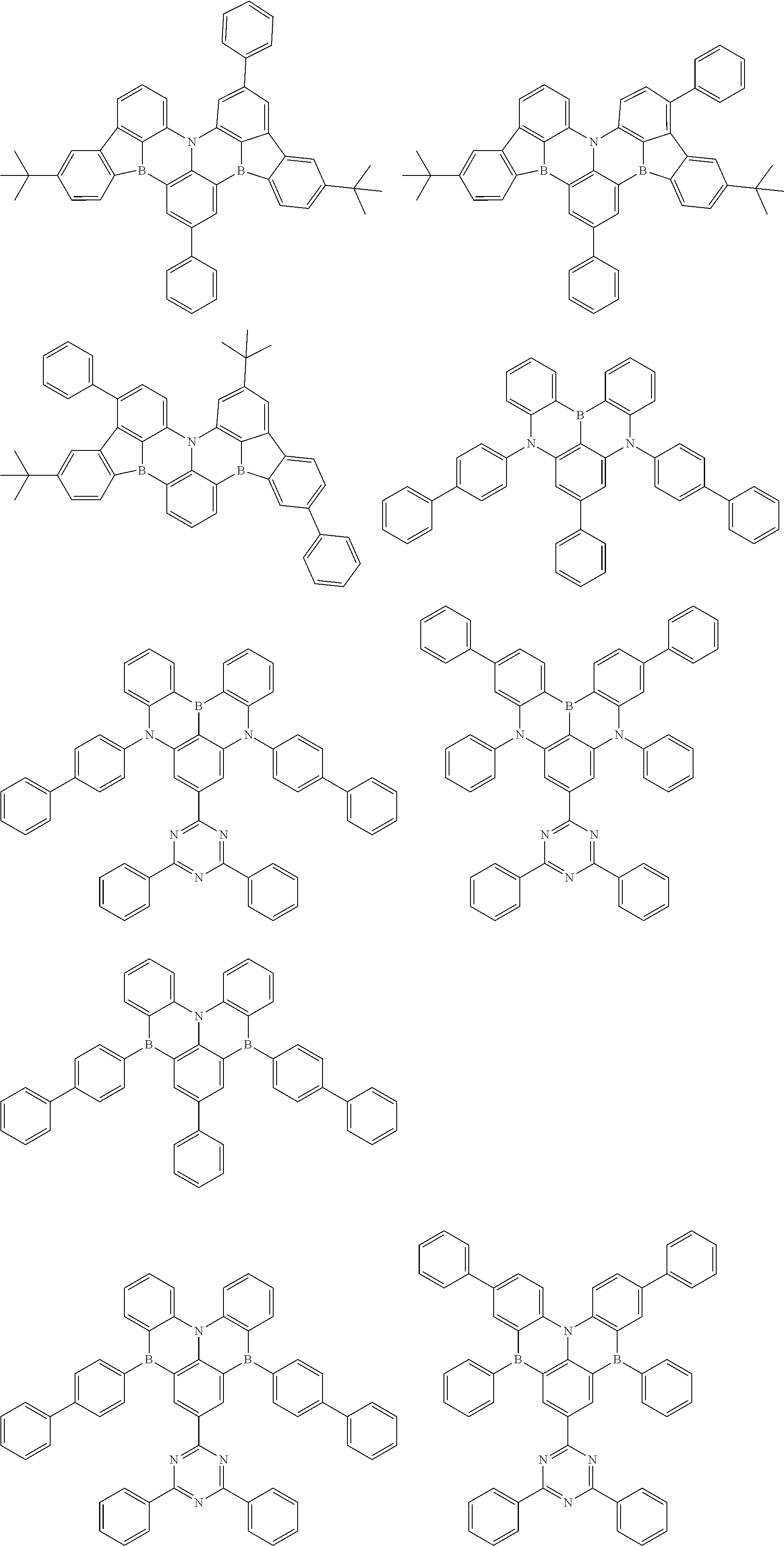

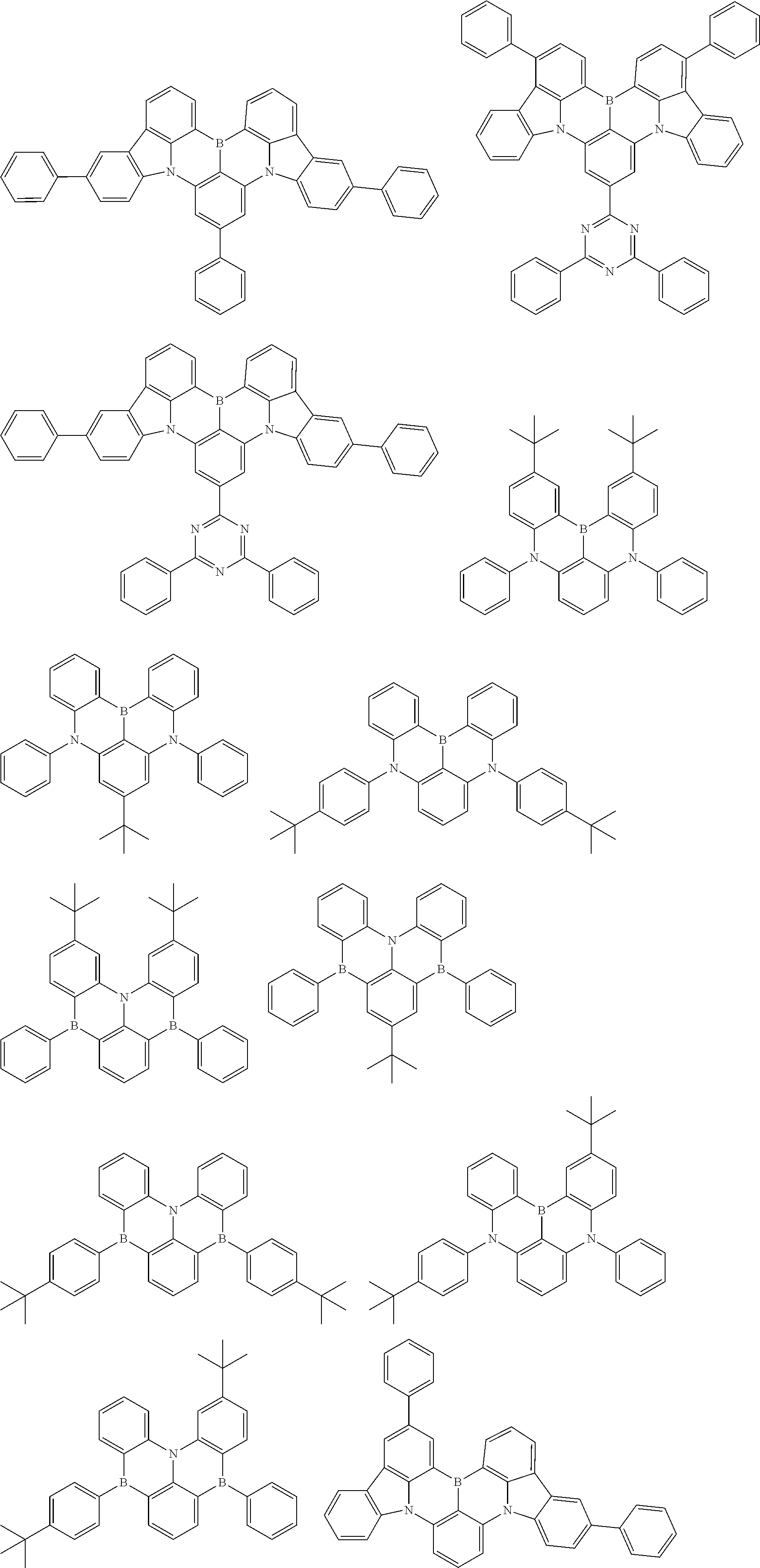

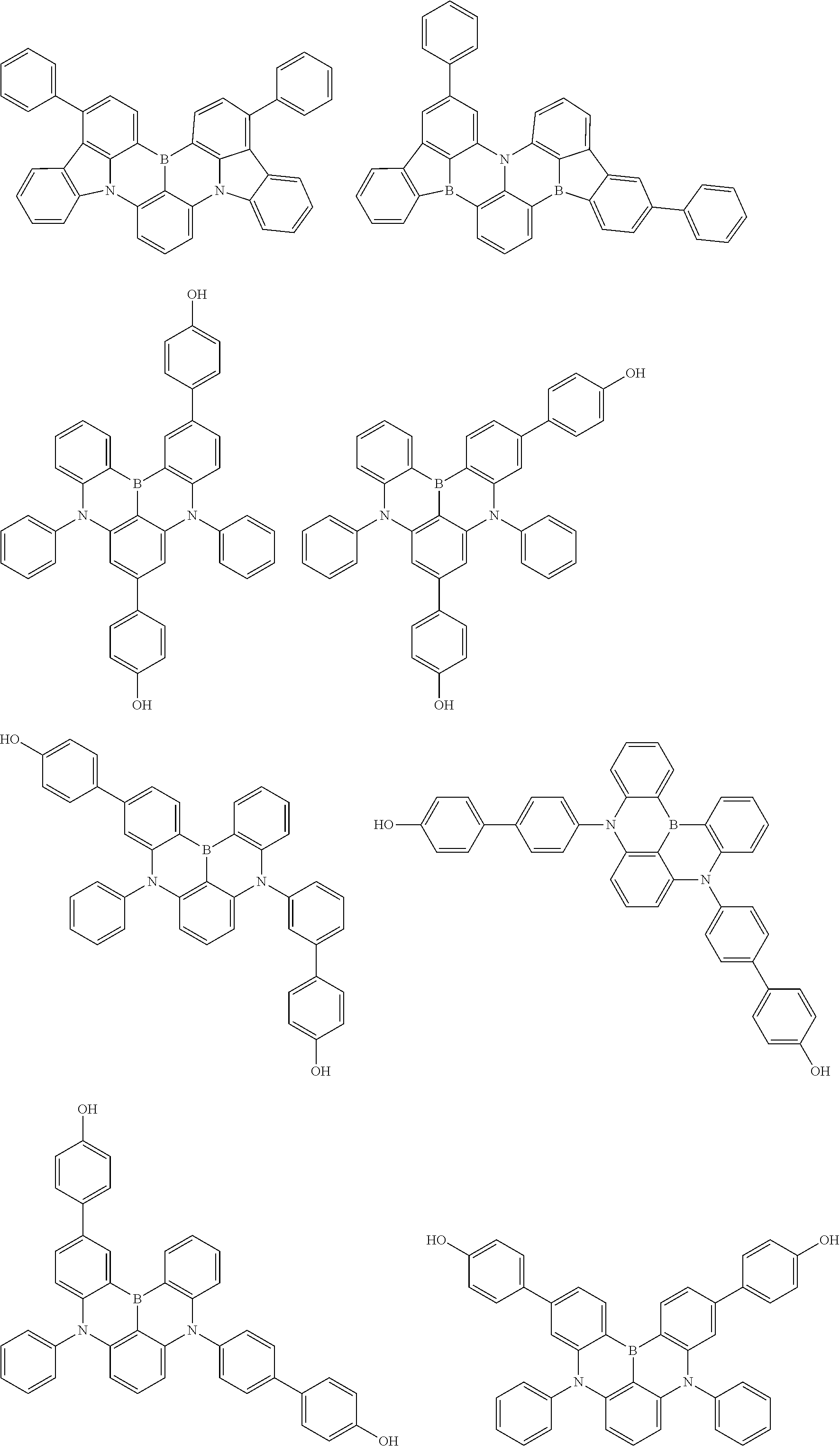

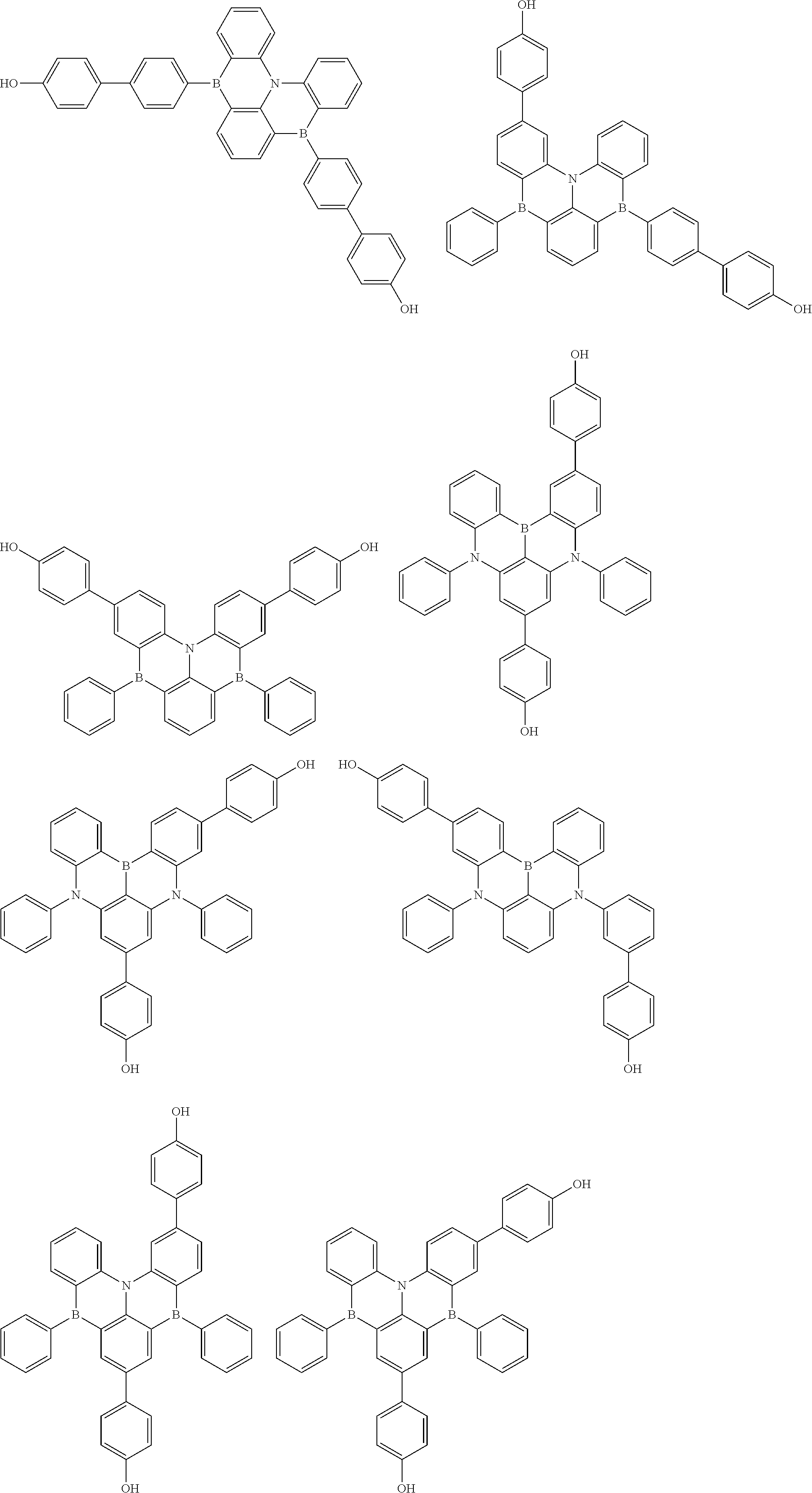

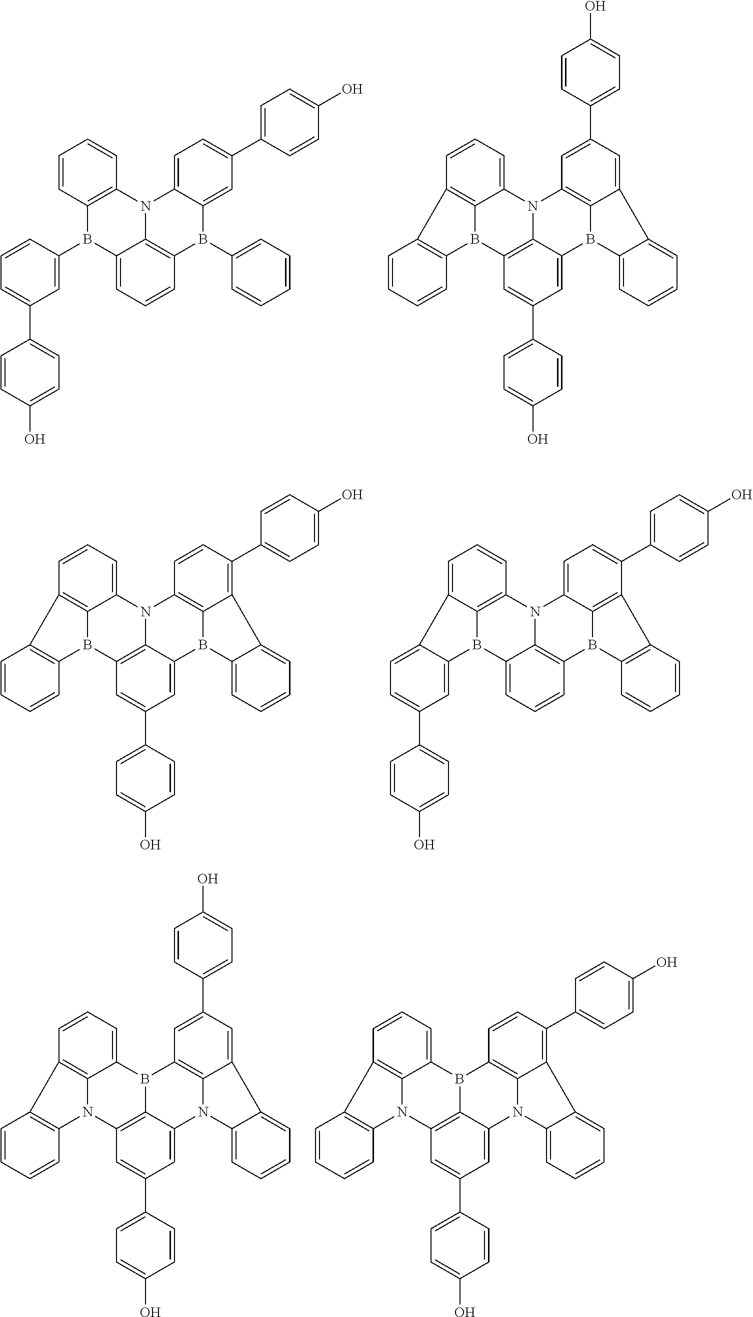

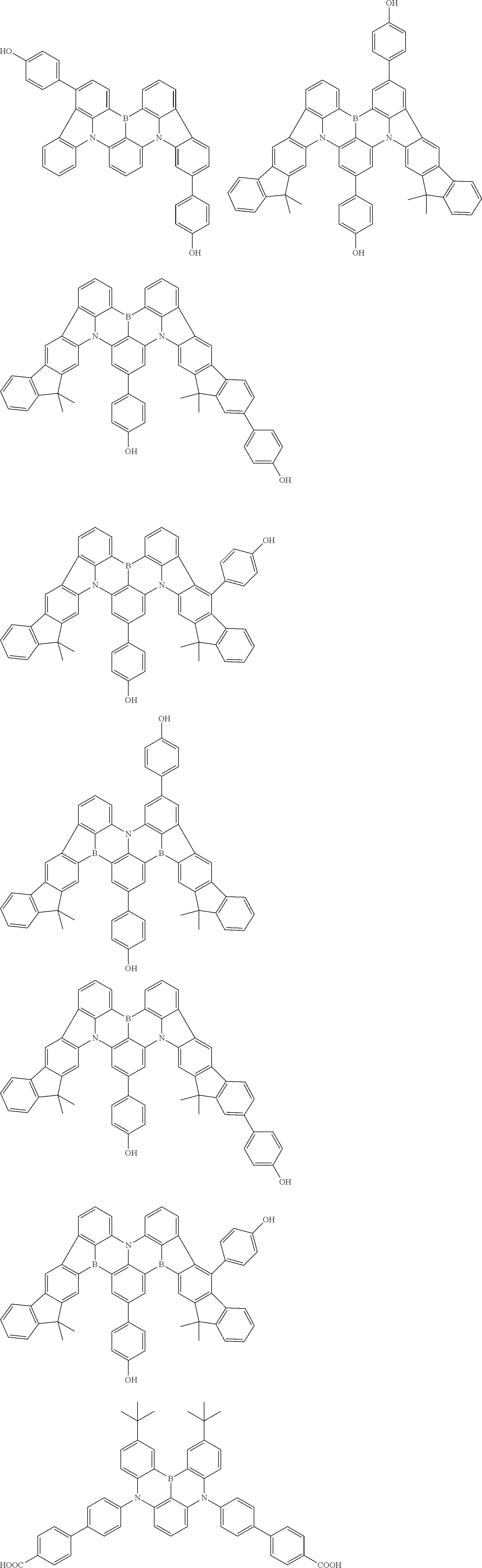

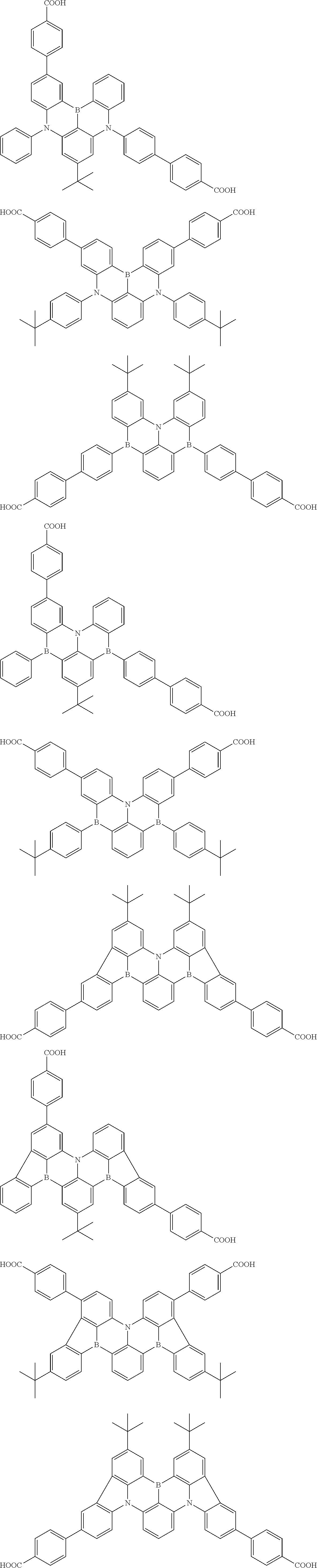

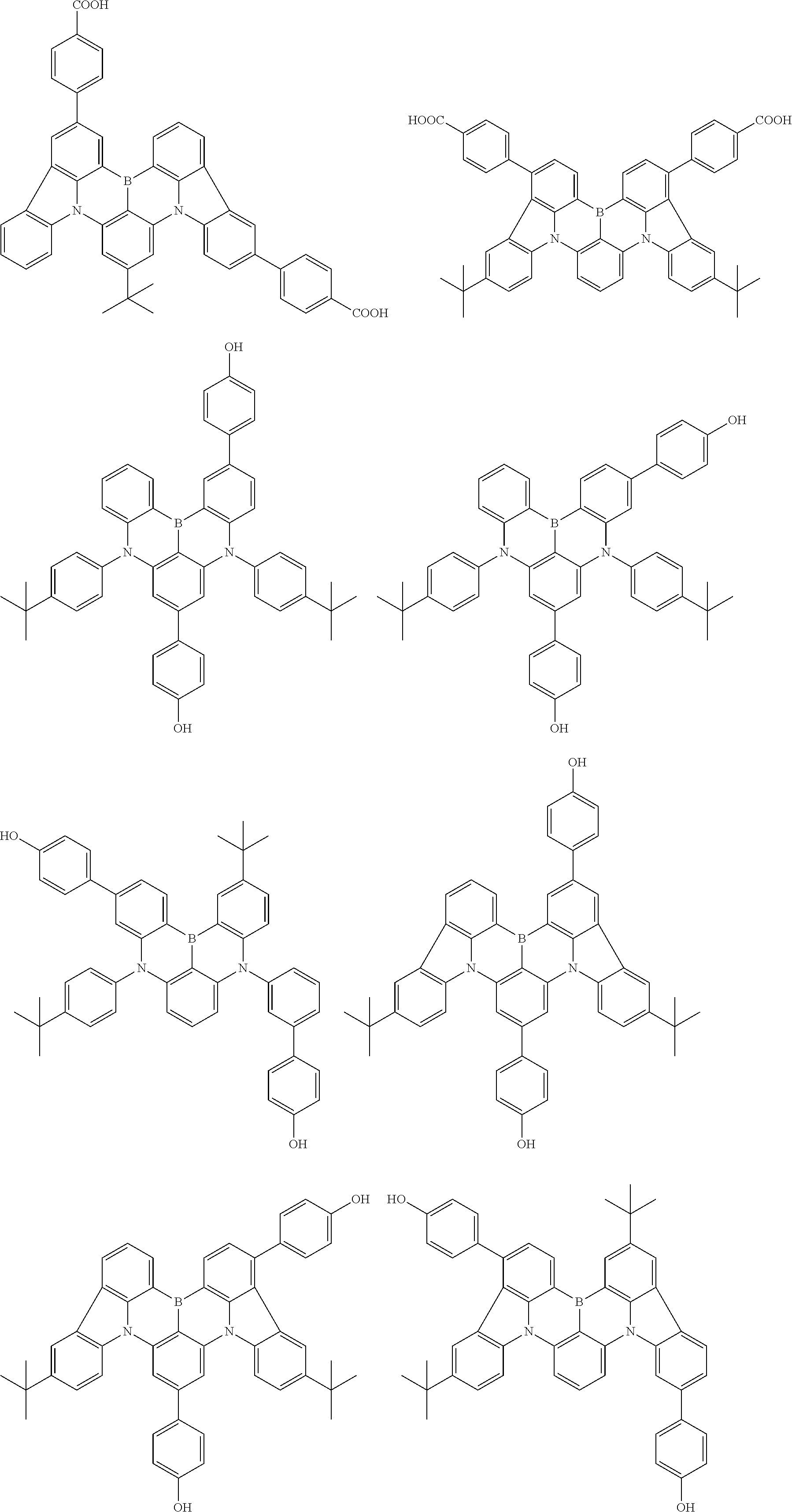

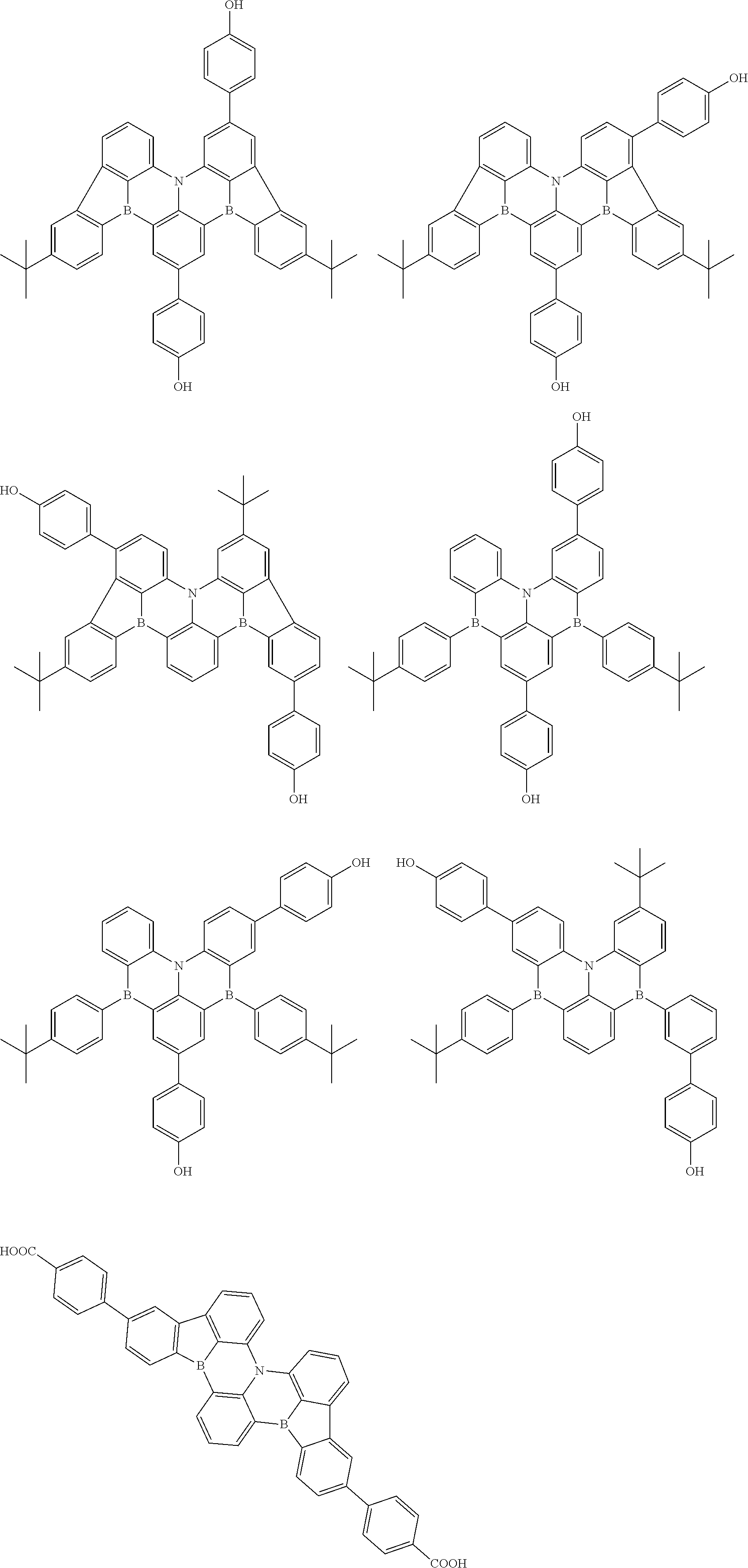

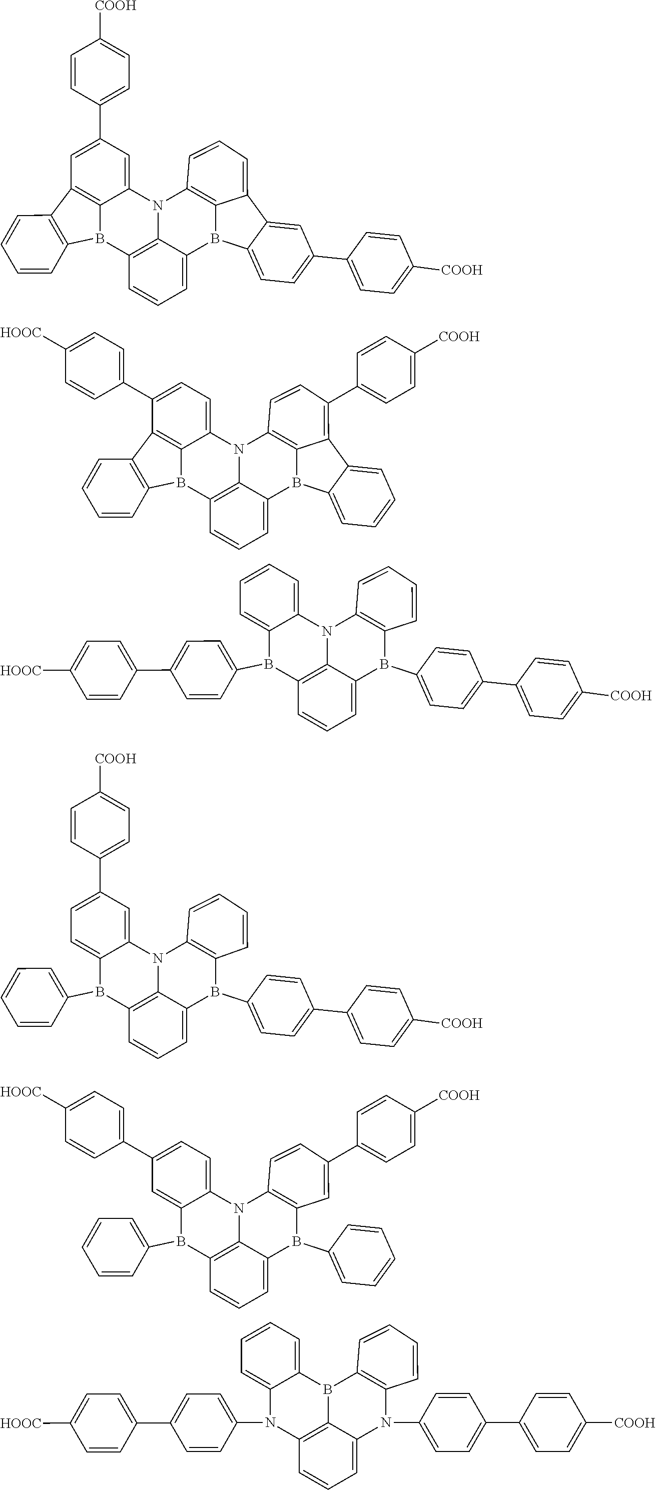

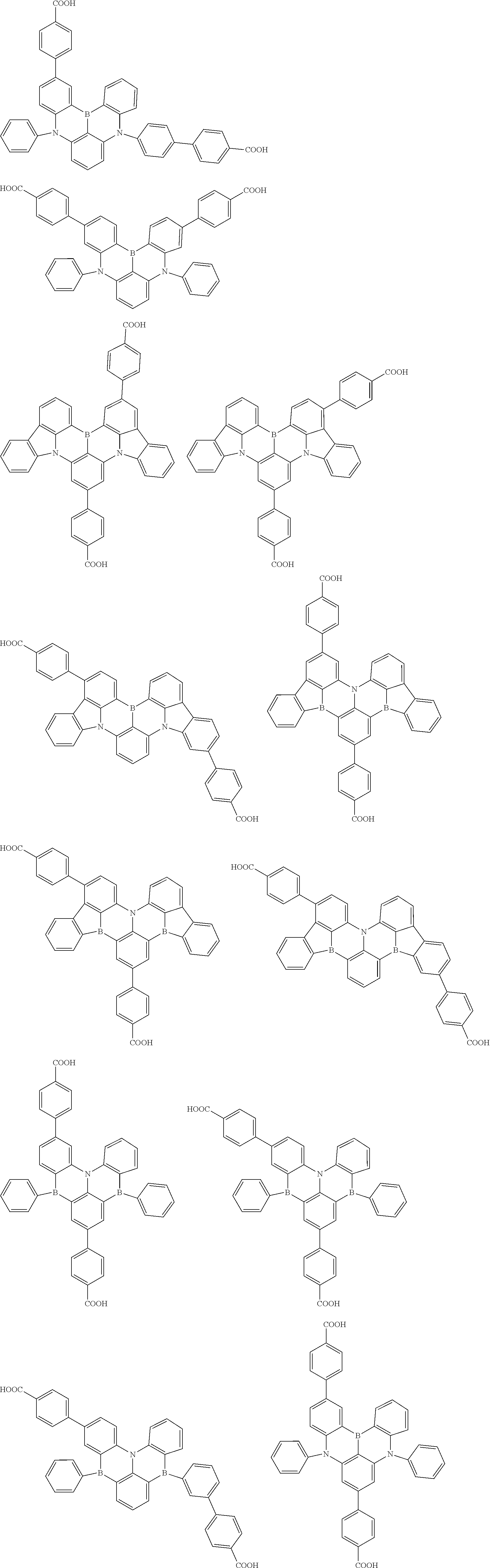

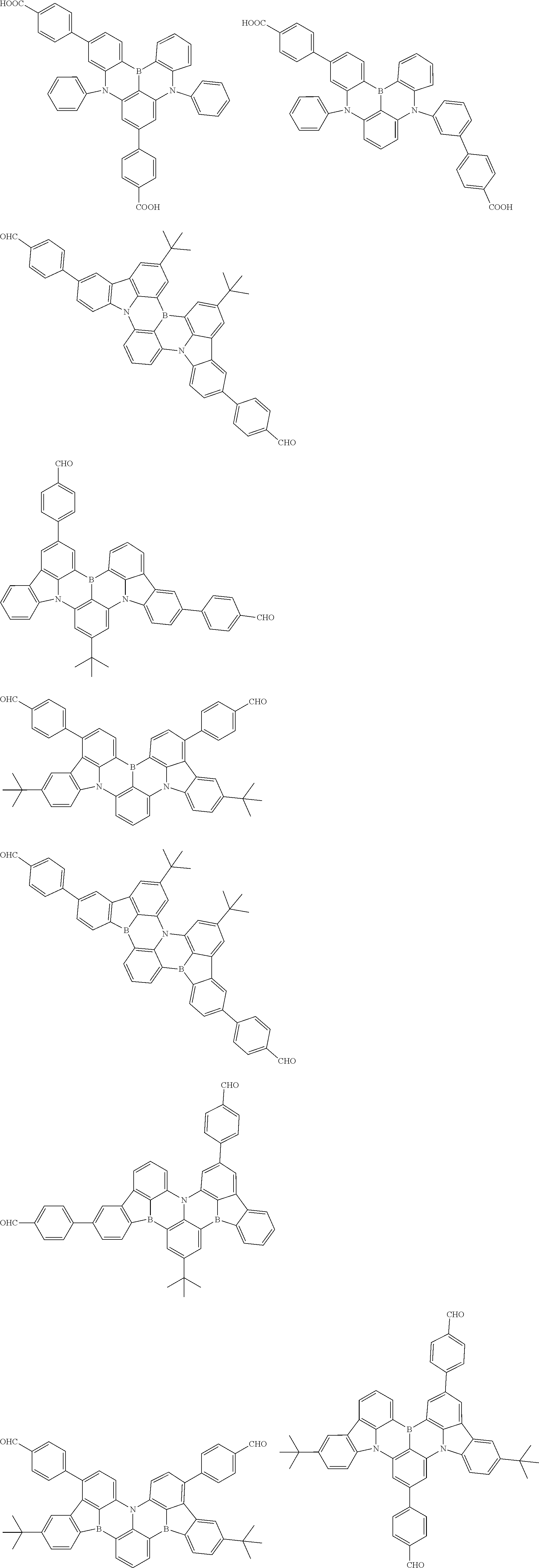

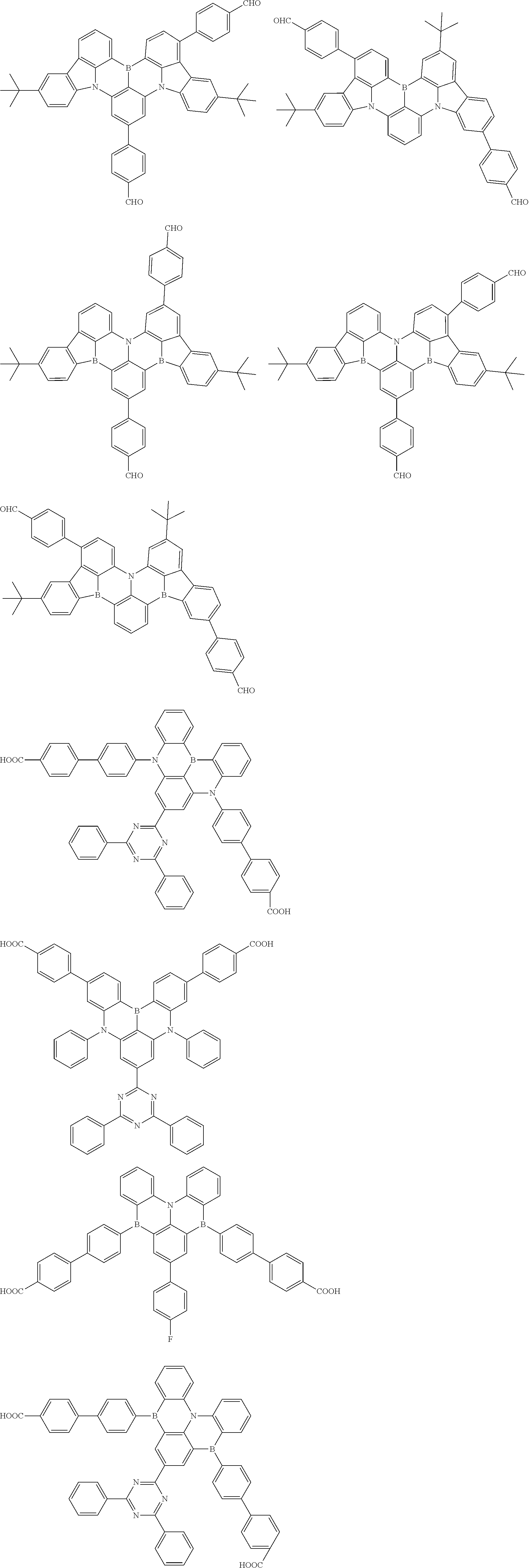

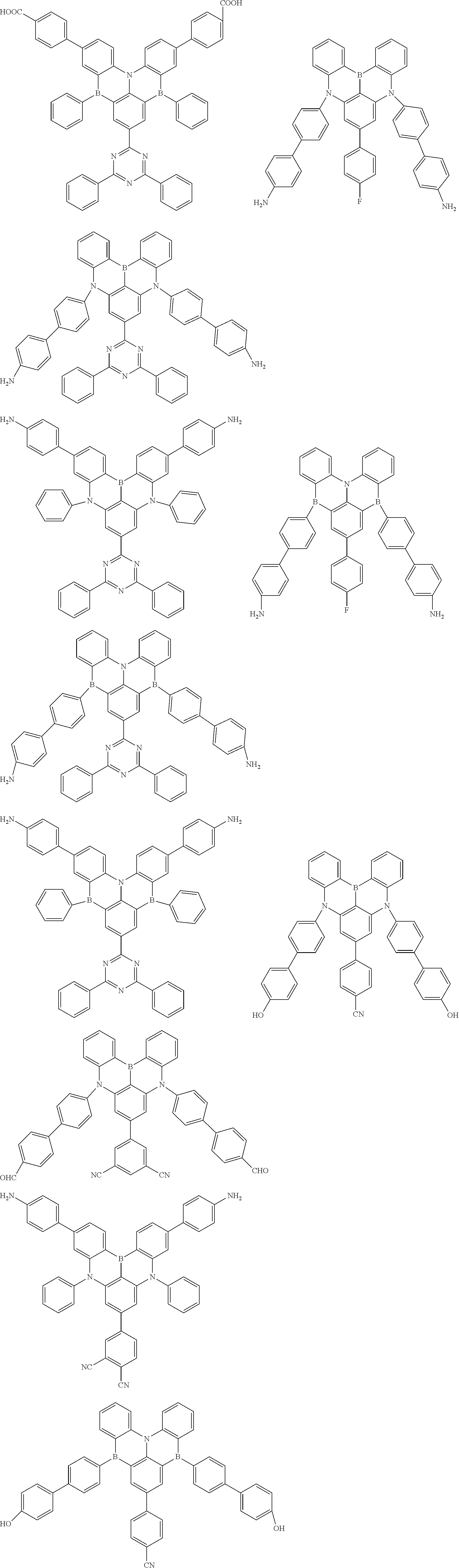

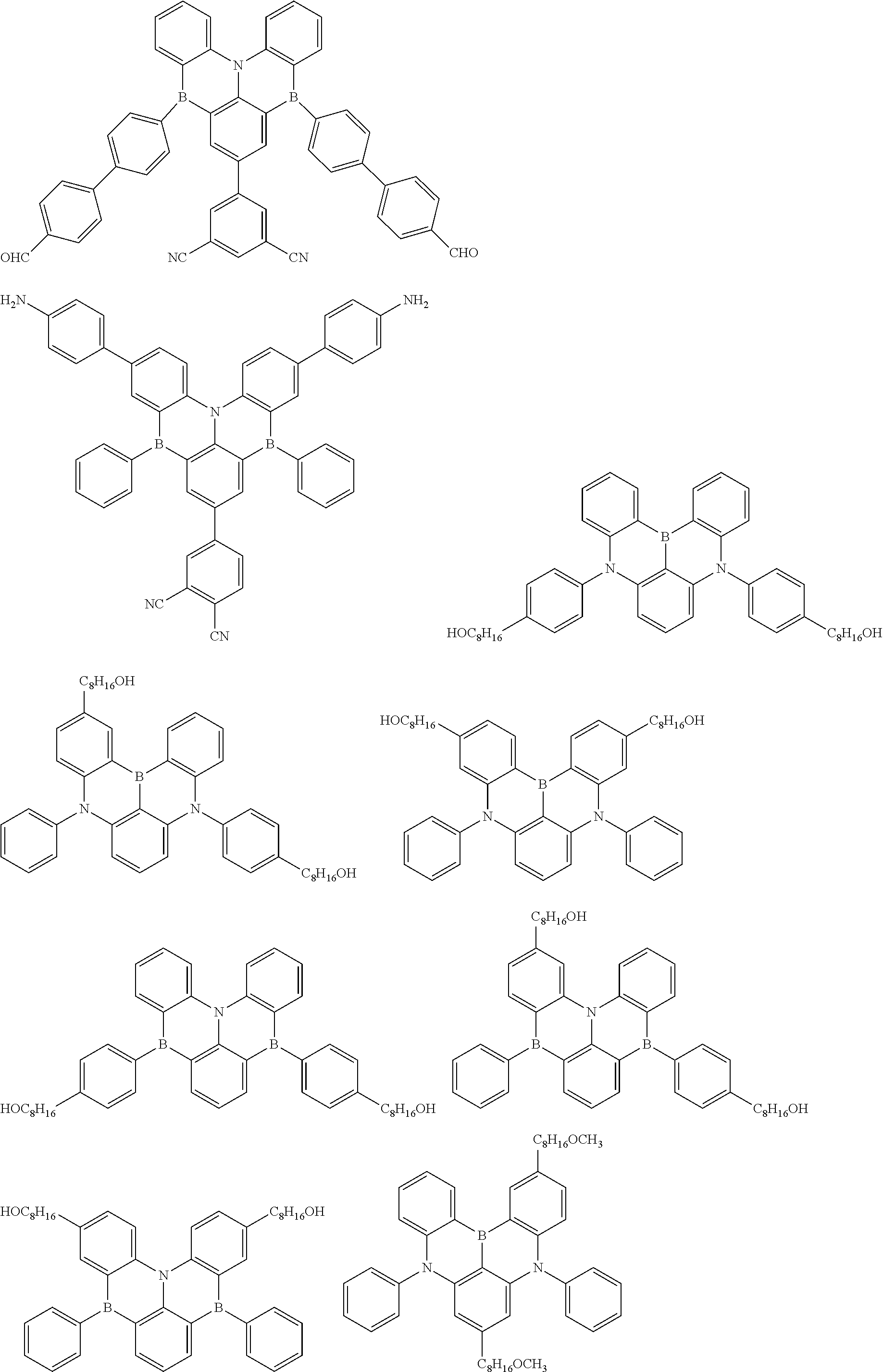

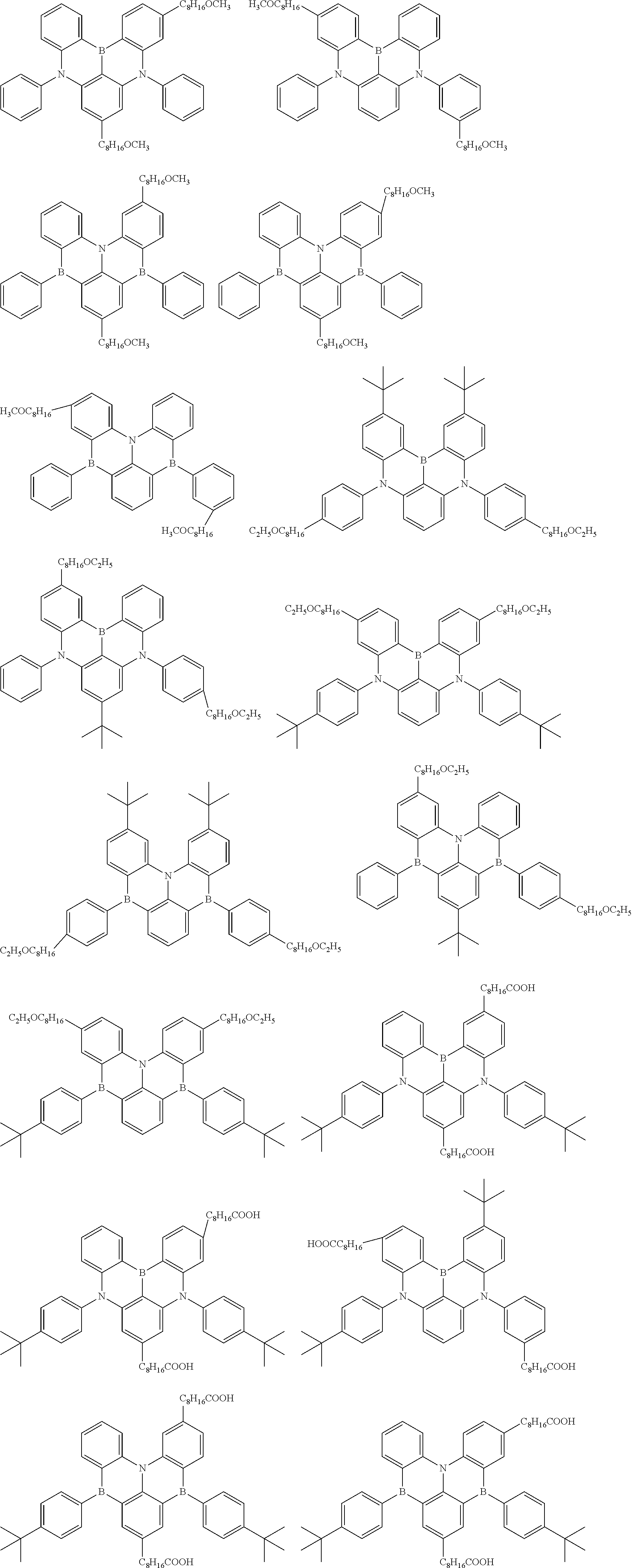

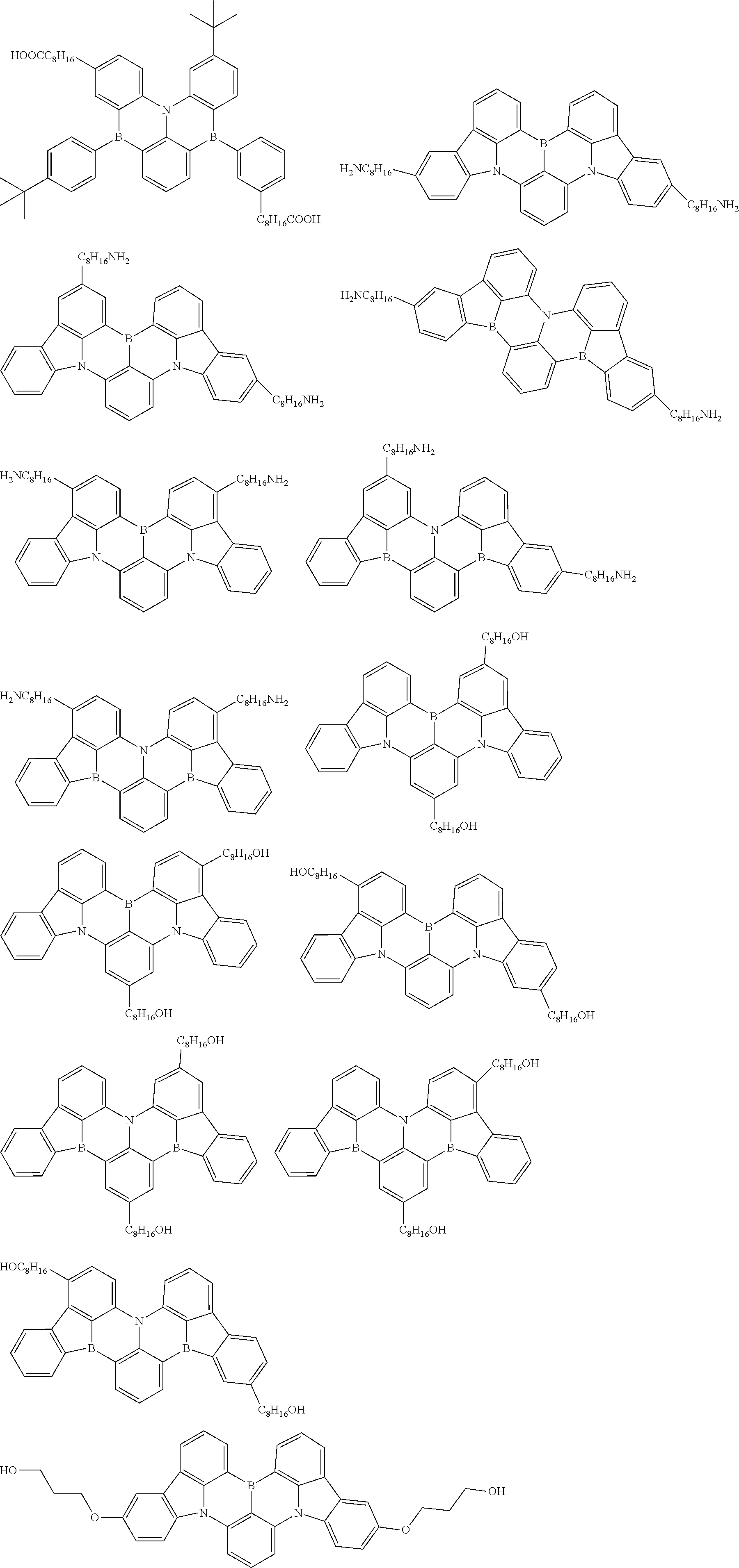

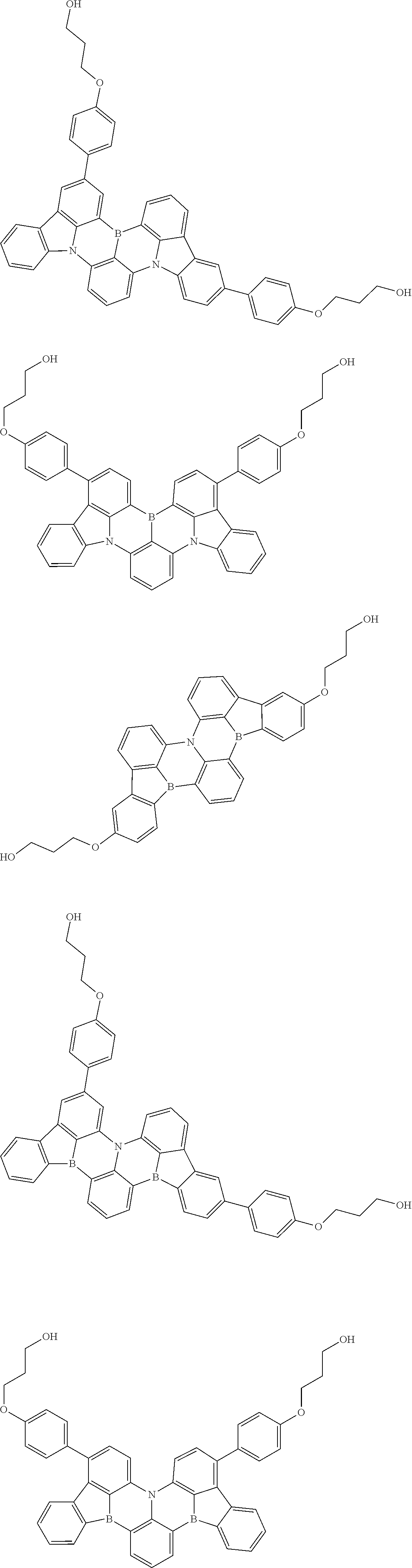 [SMILES: OCCCOc1ccc(-c2ccc3c(c2)c2cccc4c2n3-c2cccc3c2B4c2cc(-c4ccc(OCCCO)cc4)cc4c5ccccc5n-3c24)cc1 ; OCCCOc1ccc(-c2ccc3c4c2c2ccccc2n4-c2cccc4c2B3c2ccc(-c3ccc(OCCCO)cc3)c3c5ccccc5n-4c23)cc1 ; OCCCOc1ccc2c(c1)-c1cccc3c1B2c1cccc2c1N3c1cccc3c1B2c1ccc(OCCCO)cc1-3 ; OCCCOc1ccc(-c2ccc3c(c2)-c2cccc4c2B3c2cccc3c2N4c2cc(-c4ccc(OCCCO)cc4)cc4c2B3c2ccccc2-4)cc1 ; OCCCOc1ccc(-c2ccc3c4c2-c2ccccc2B4c2cccc4c2N3c2ccc(-c3ccc(OCCCO)cc3)c3c2B4c2ccccc2-3)cc1]

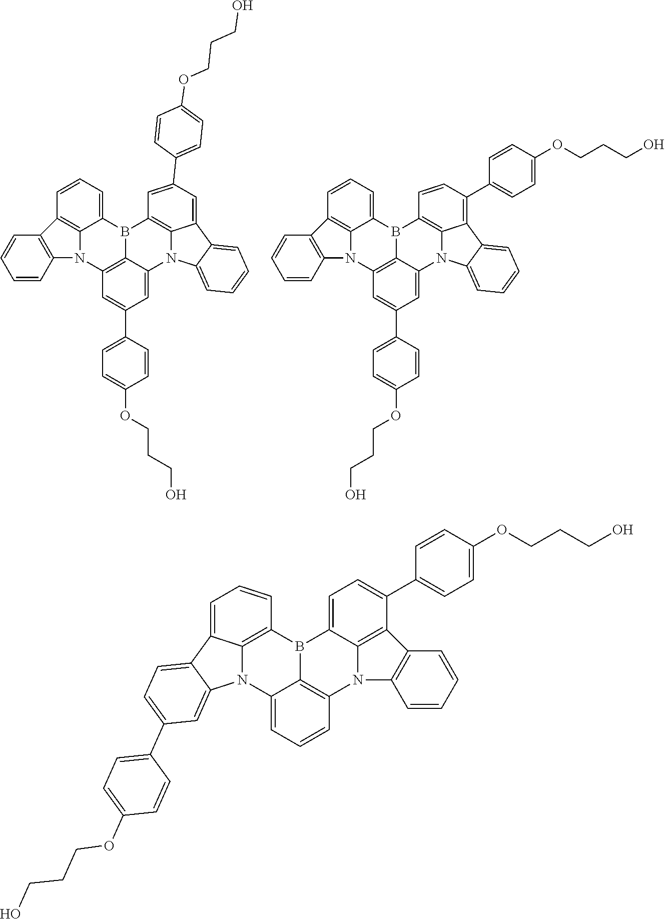

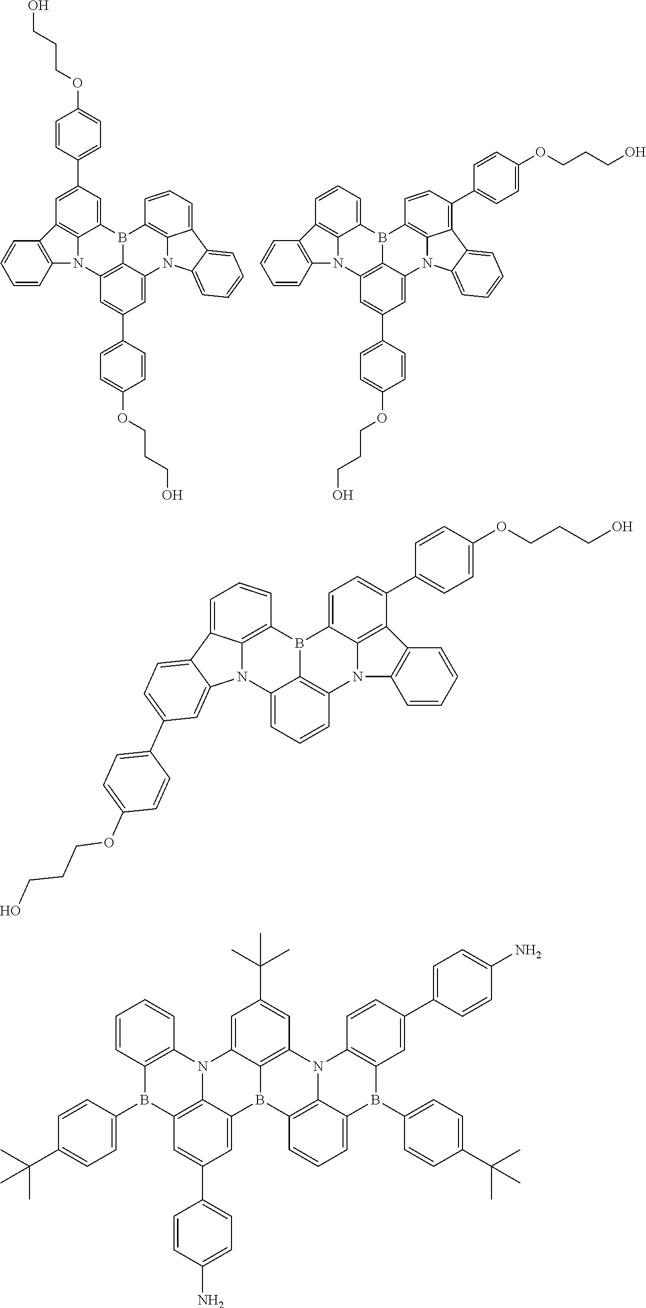

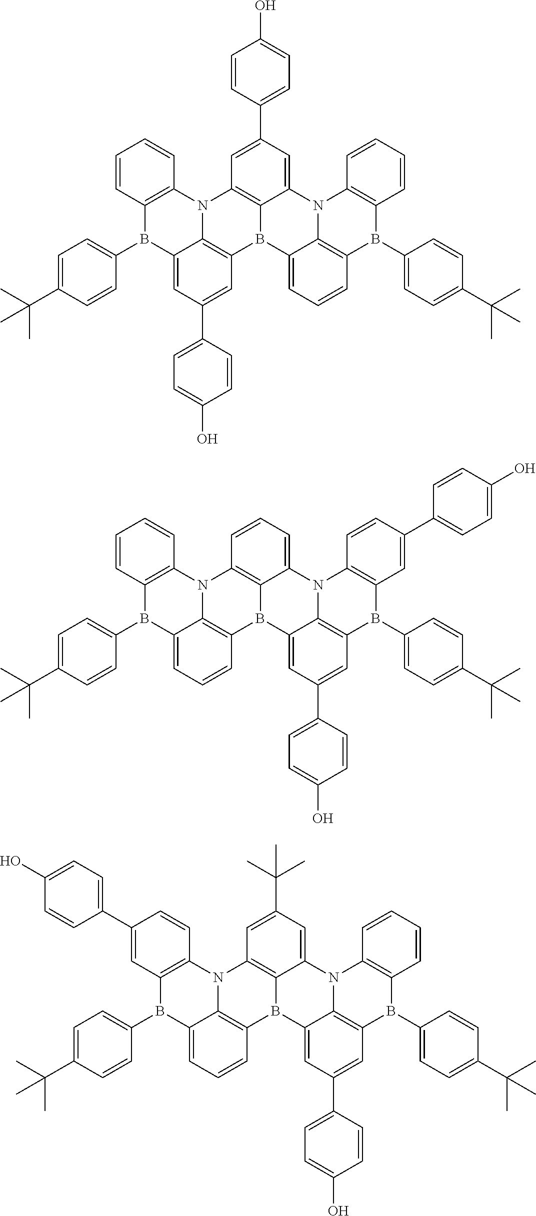

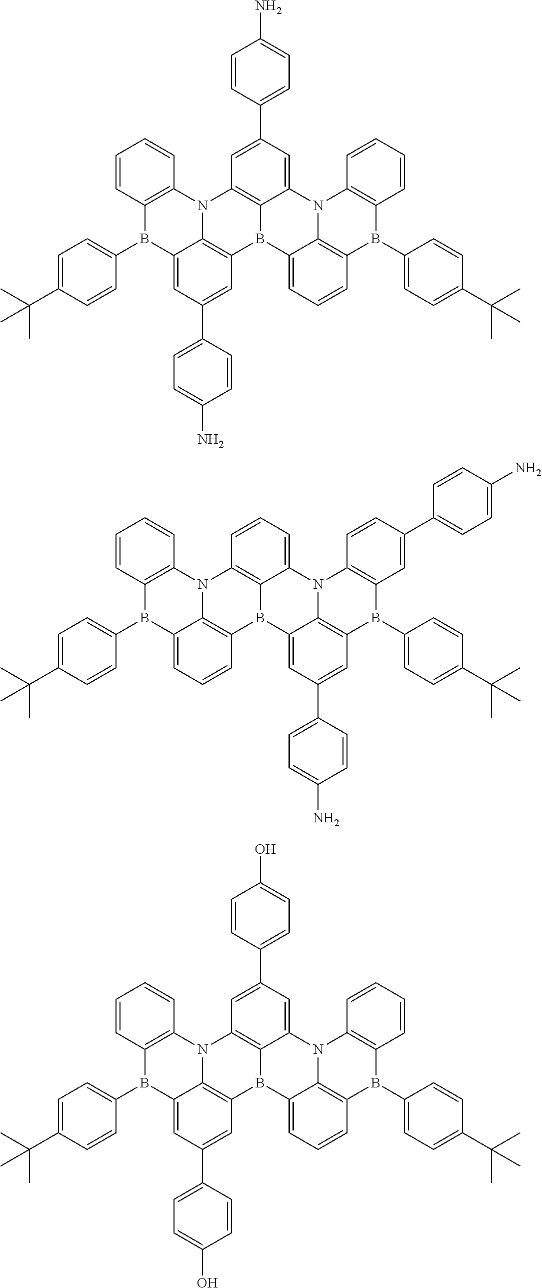

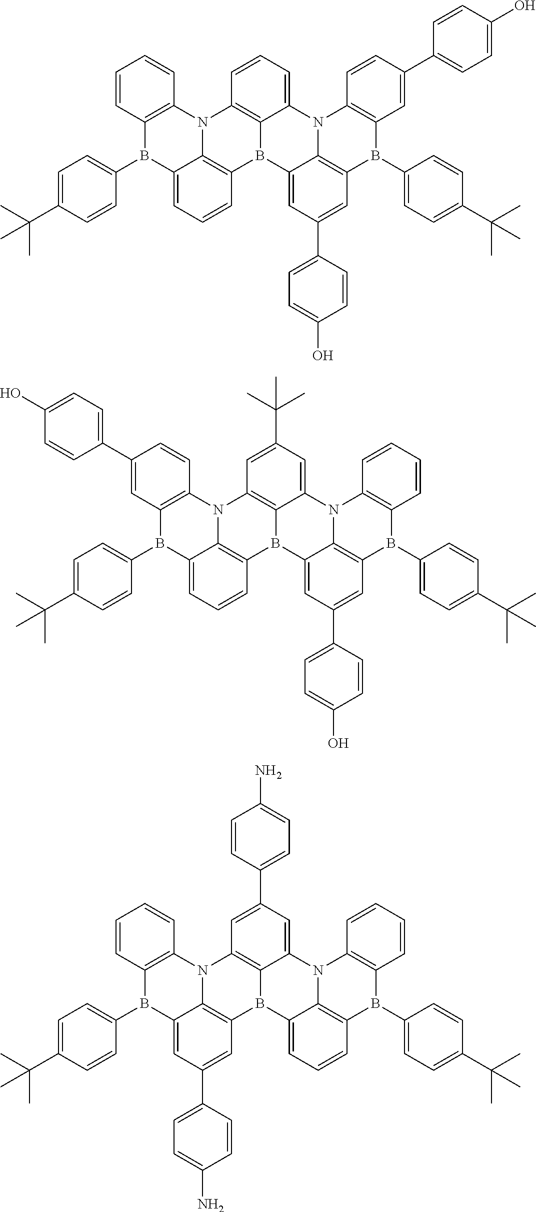

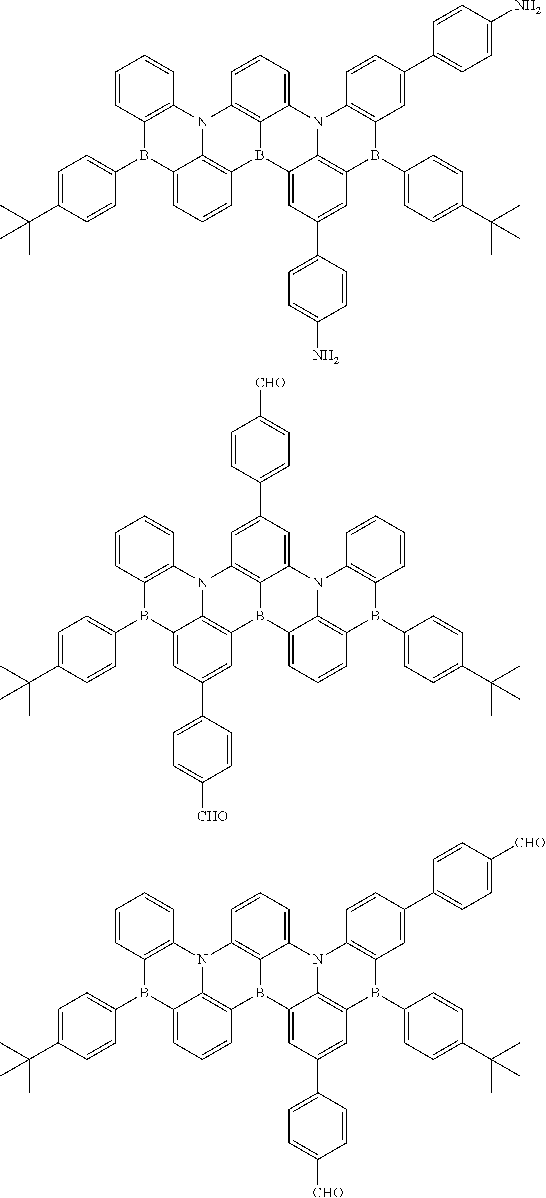

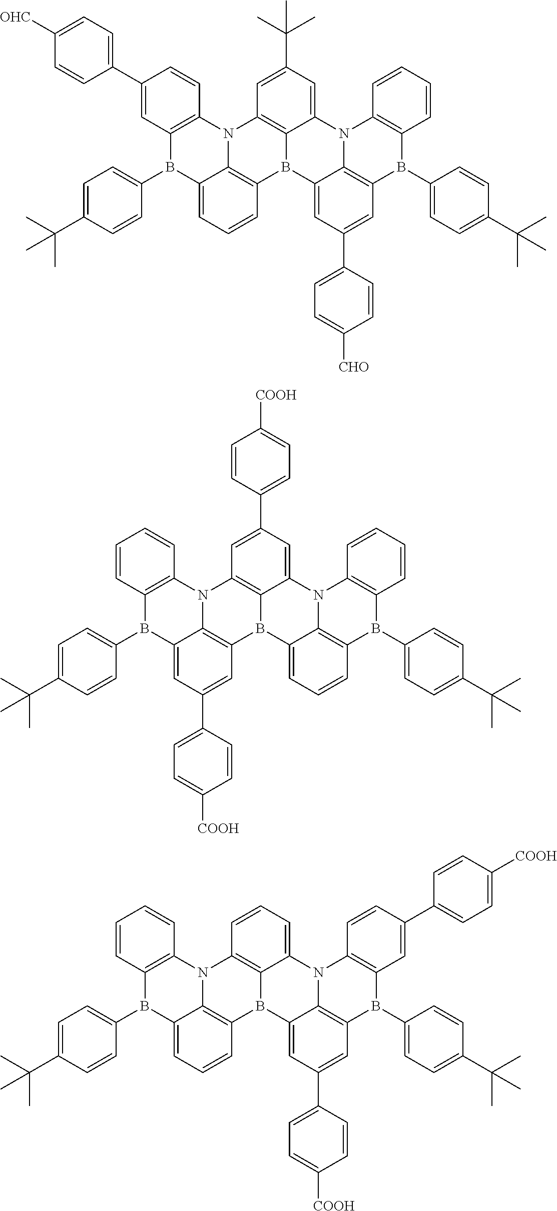

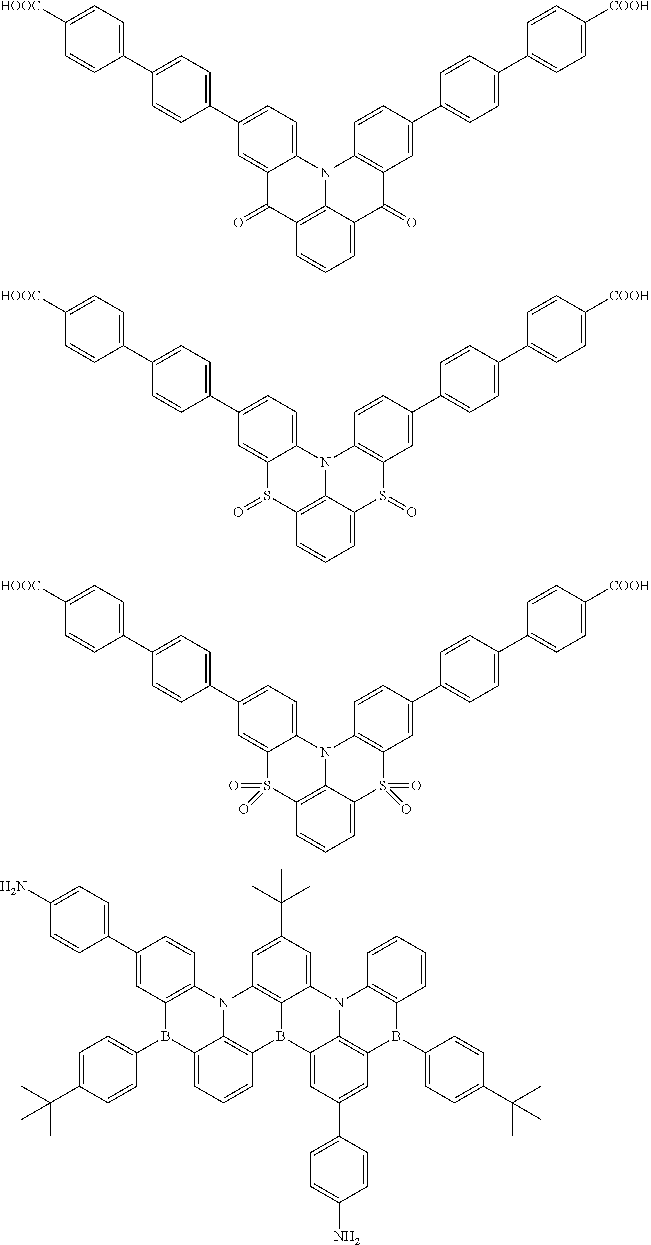

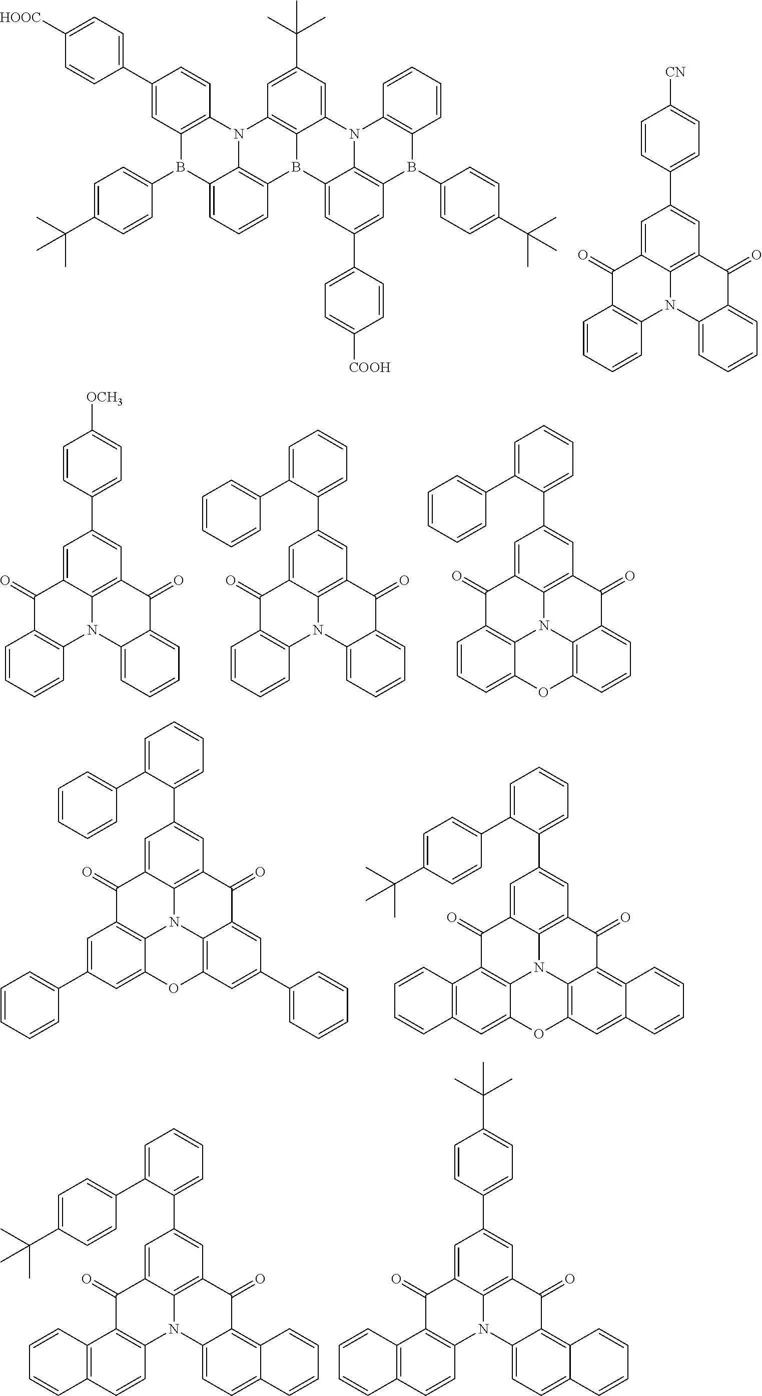

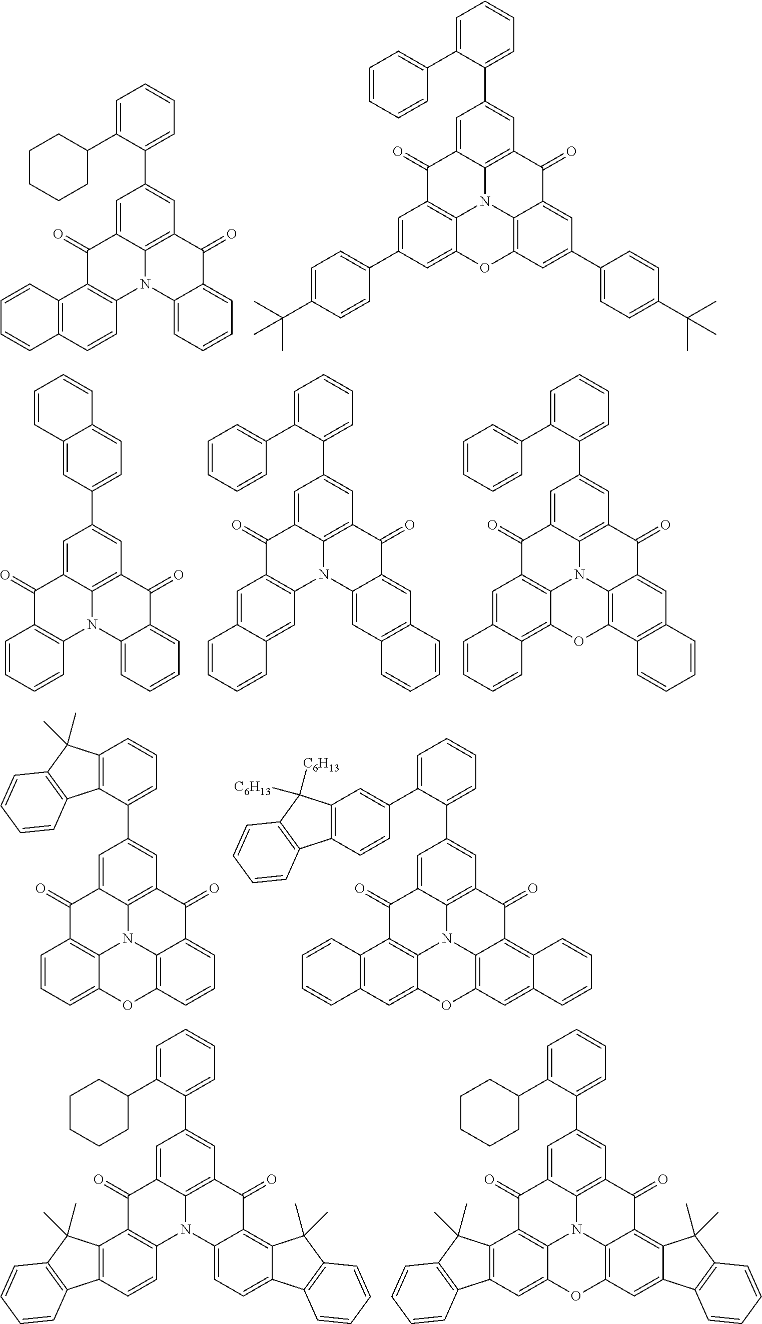

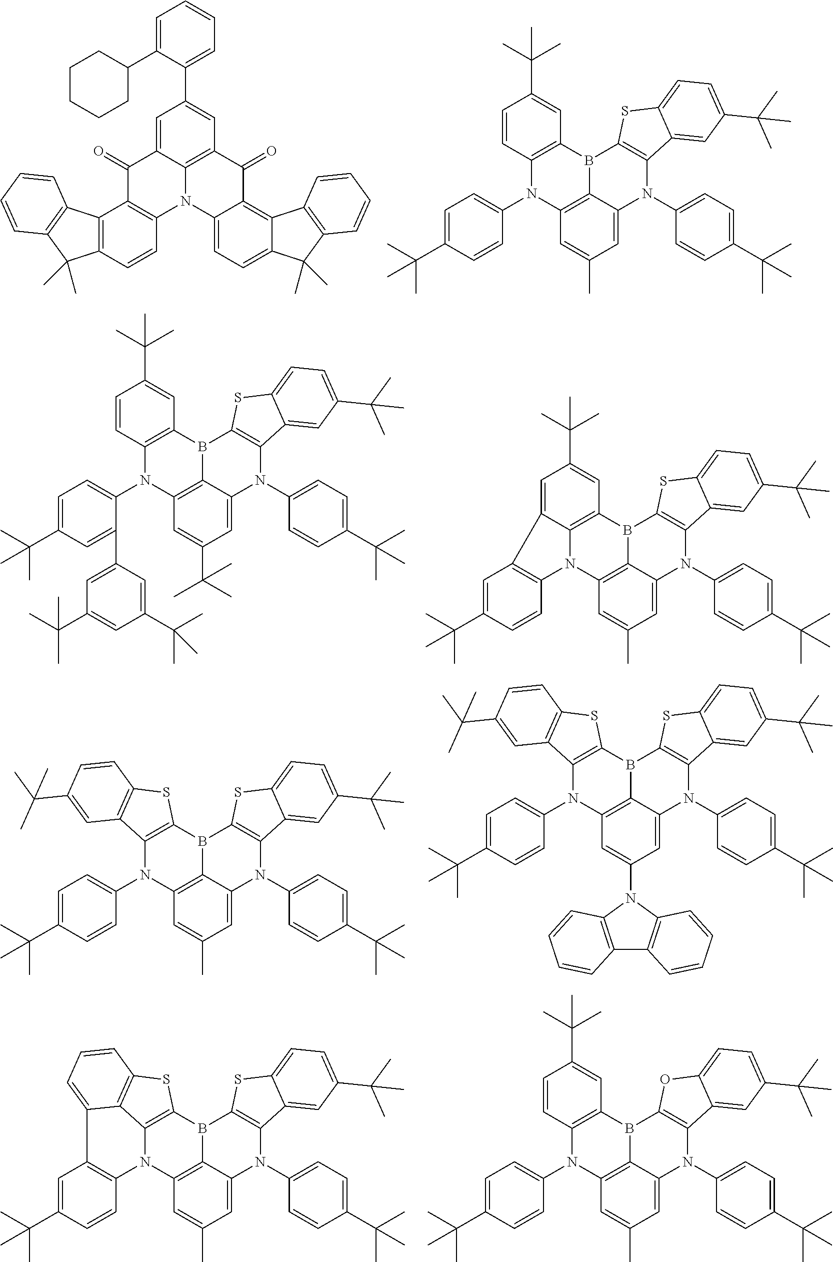

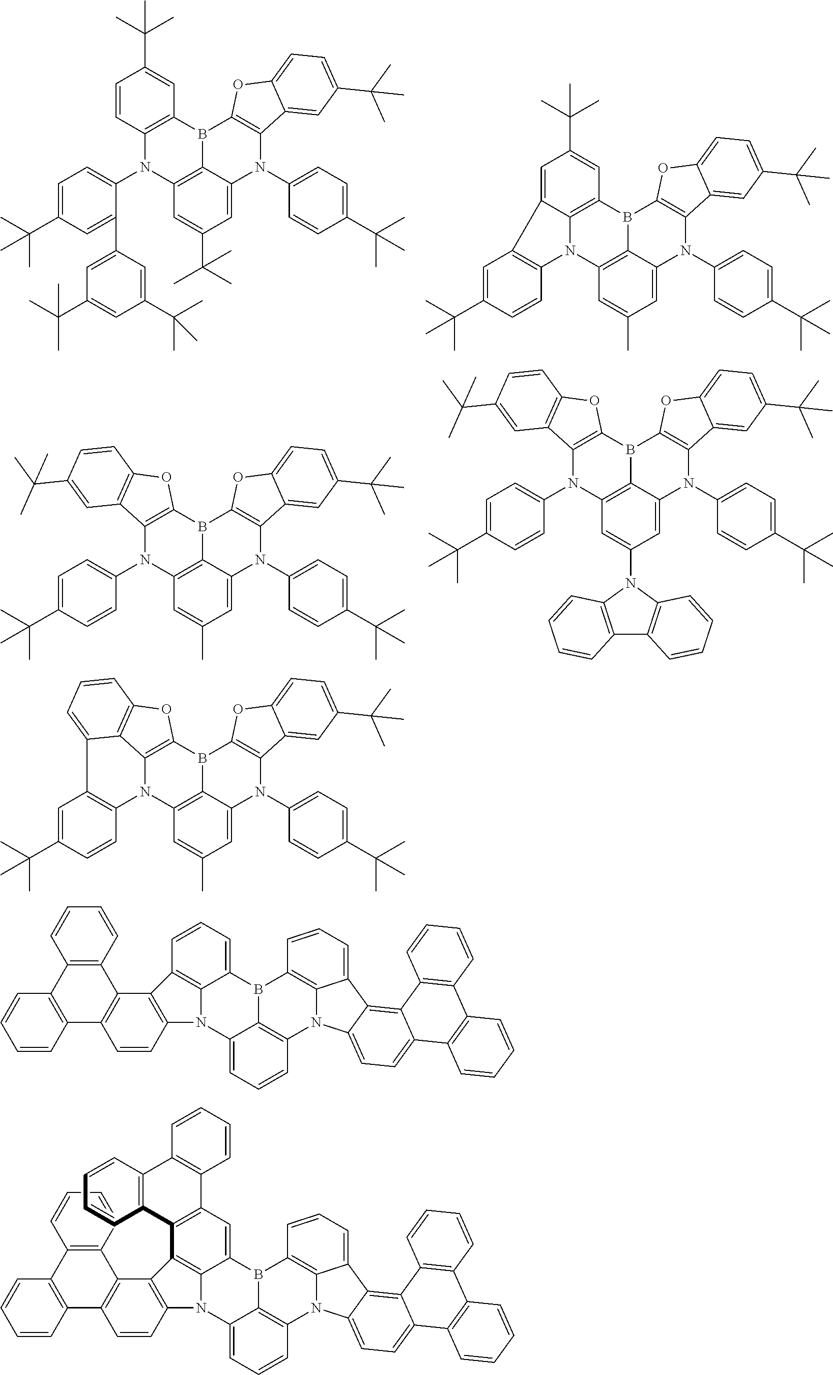

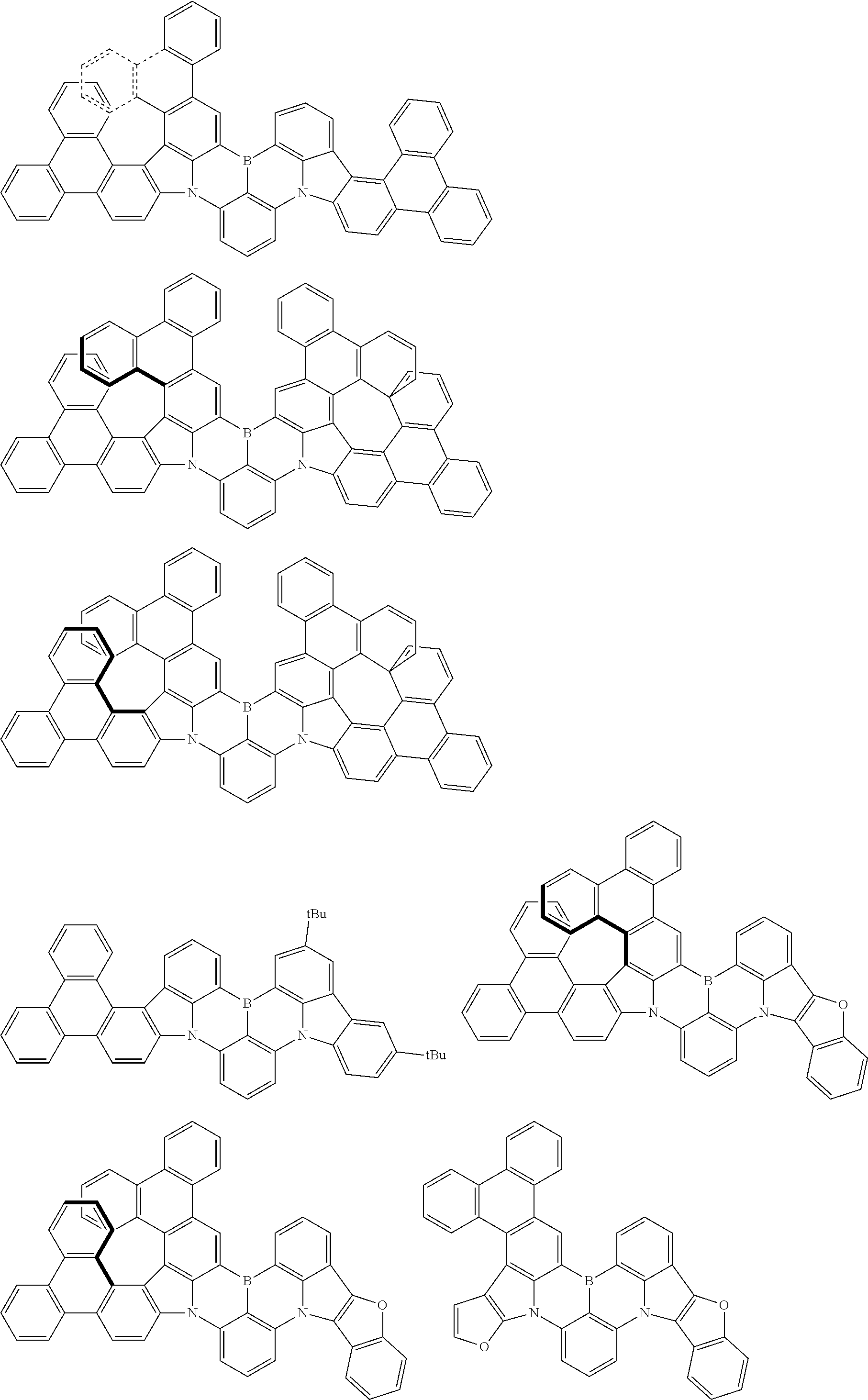

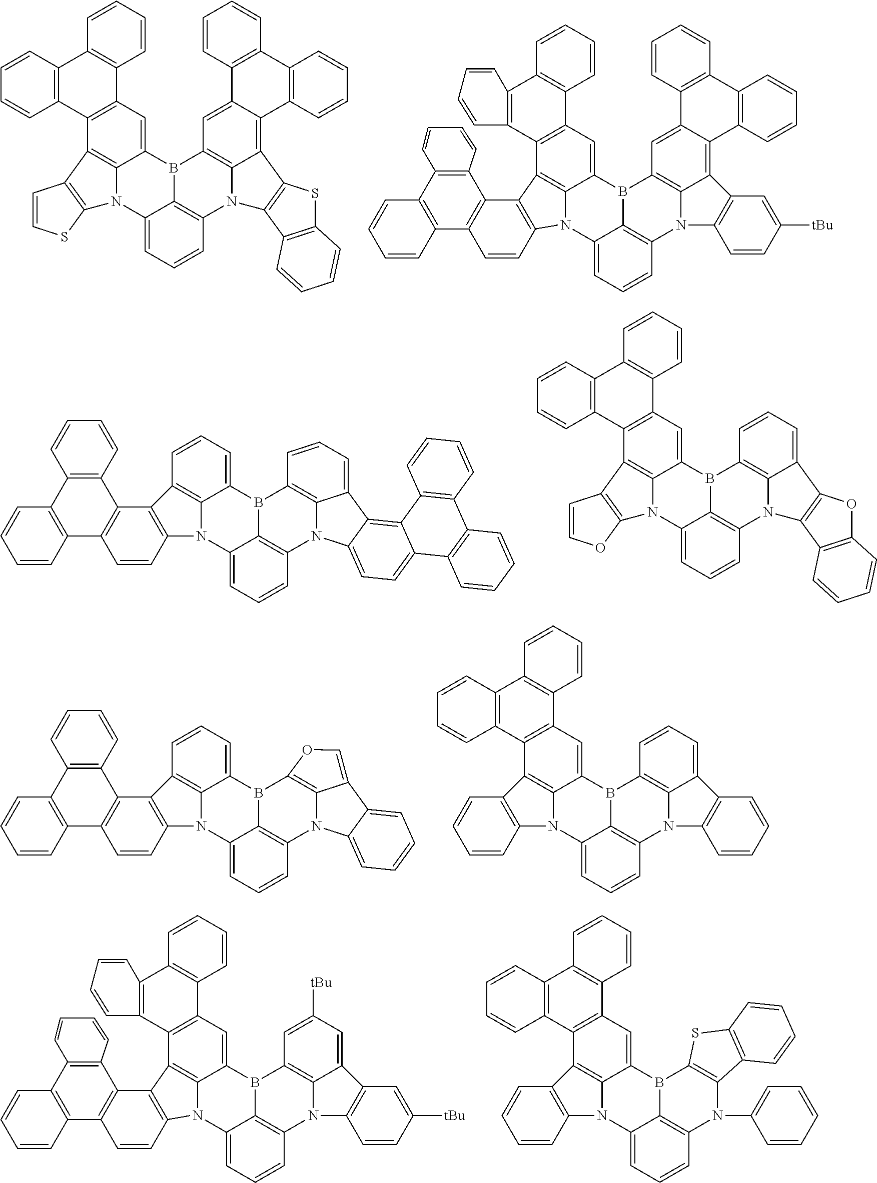

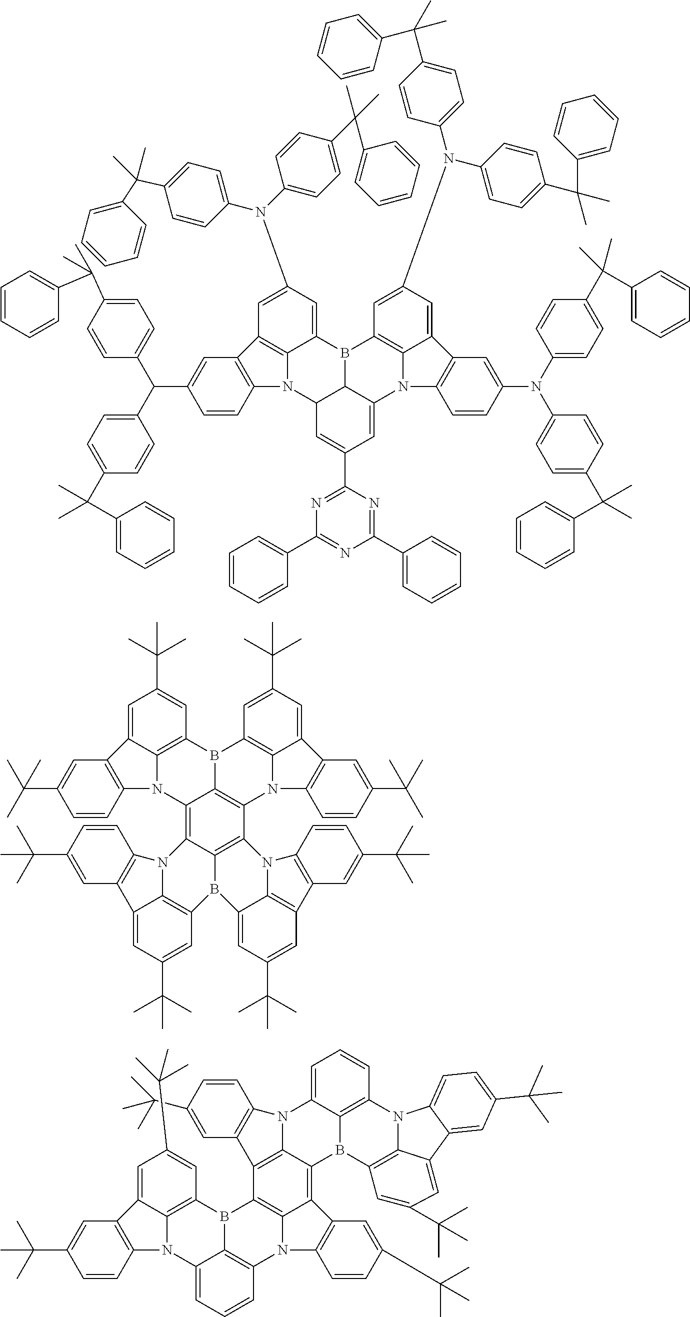

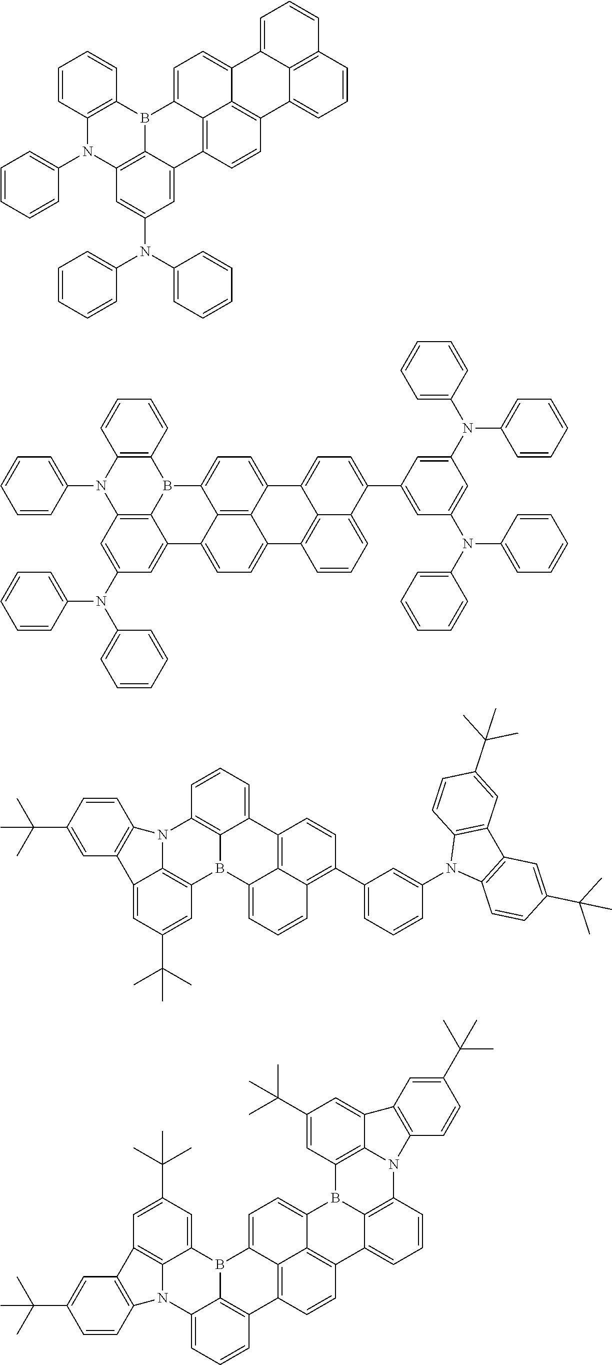

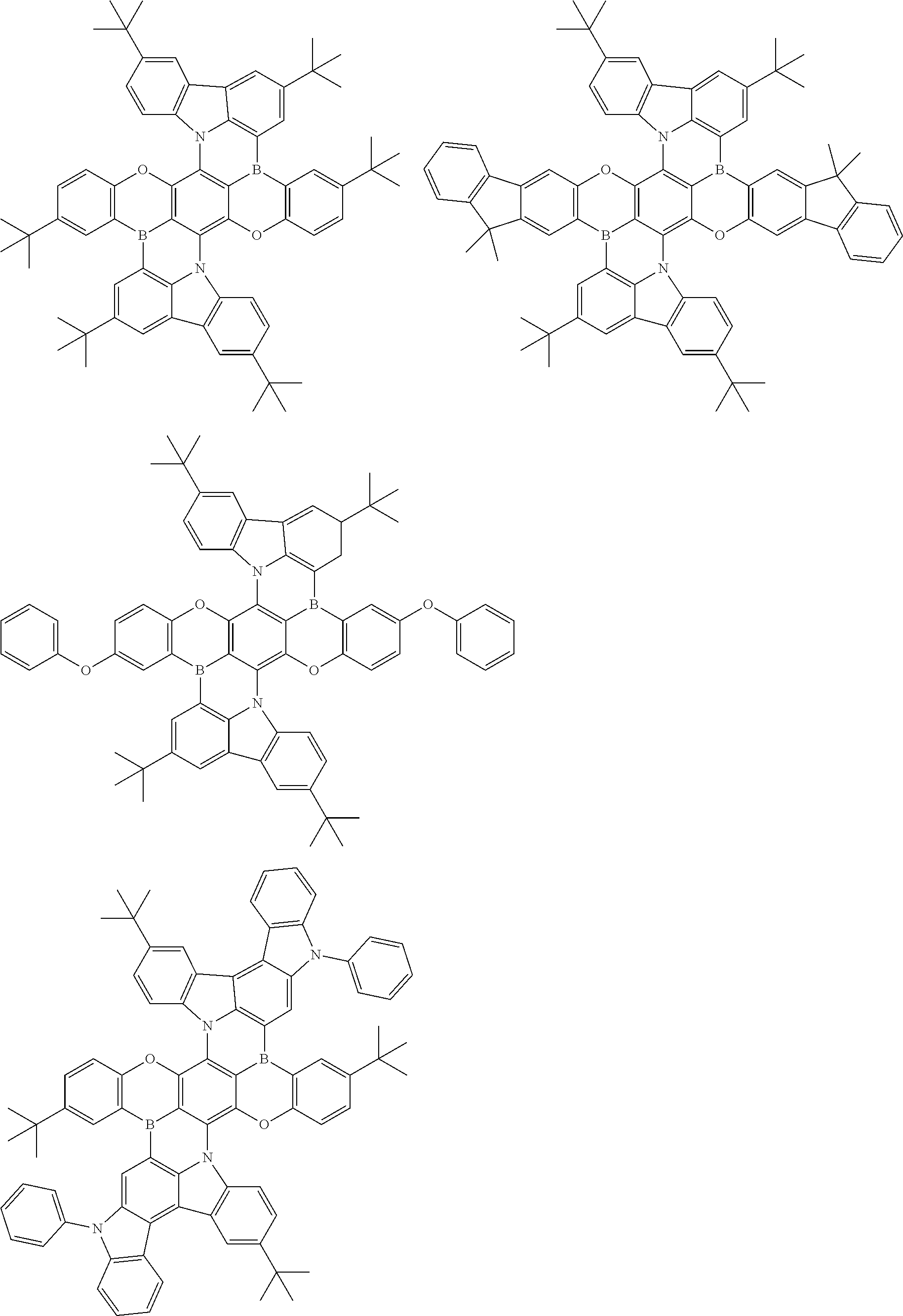

In some embodiments, the organic compound H and/or the emitter E comprises at least one cross-linkable group, as disclosed in International Application Publication No. WO202207843A1, which is hereby incorporated by reference in its entirety. The advantage is that when the resin prepolymer is copolymerized or homopolymerized, the emitter E can at least partially participate in the polymerisation.

In some embodiments, the organic compound H and/or the emitter E comprises at least two cross-linkable groups.

In some embodiments, the organic compound H and/or the emitter E comprises at least three cross-linkable groups.

In some embodiments, the organic compounds H may be polymerized to form a polymer. That is, the formulation as described herein comprises an organic compound H and an emitter E as described herein, or comprises a polymer and an emitter E as described herein, or comprises an organic compound H, a polymer, and an emitter E as described herein. Preferably, the polymer is a side chain polymer

In some embodiments, the emitter E is a polymer comprising at least one repeating structural unit of formula (1) or formula (2). Preferably, the polymer is a side chain polymer as disclosed in International Application Publication No. WO2022078456A1, which is hereby incorporated by reference in its entirety.

In some embodiments, for the purposes of the present disclosure, the emitter E may be further selected from compounds (i.e., Bodipy derivatives) having the following structural formula:

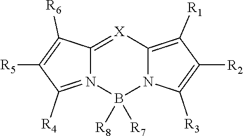

Where X is CR₉ or N; each of R¹ to R₉ is independently selected from a hydrogen, an alkyl group, a cycloalkyl group, a heterocyclic group, an alkenyl group, a cycloalkenyl group, an alkynyl group, a hydroxyl group, a sulfhydryl group, an alkoxy group, an alkylthio group, an aryl ether group, an aryl thioether group, an aryl group, a heteroaryl group, a halogen, a cyano group, an aldehyde group, a carbonyl group, a carboxyl group, an oxycarboxyl group, a carbamoyl group, an amino group, a nitro group, a silyl group, a siloxanyl group, a boranyl group, or a phosphine oxide group; and R₁-R₉ may form a fused ring and an aliphatic ring with the adjacent substituents.

Suitable examples of Bodipy derivatives include, but not limited to:

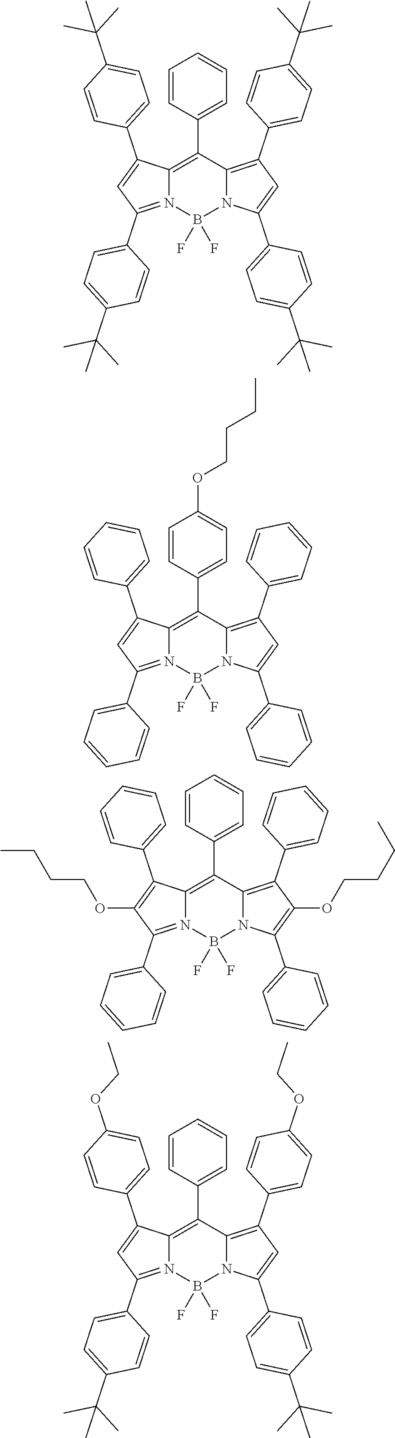

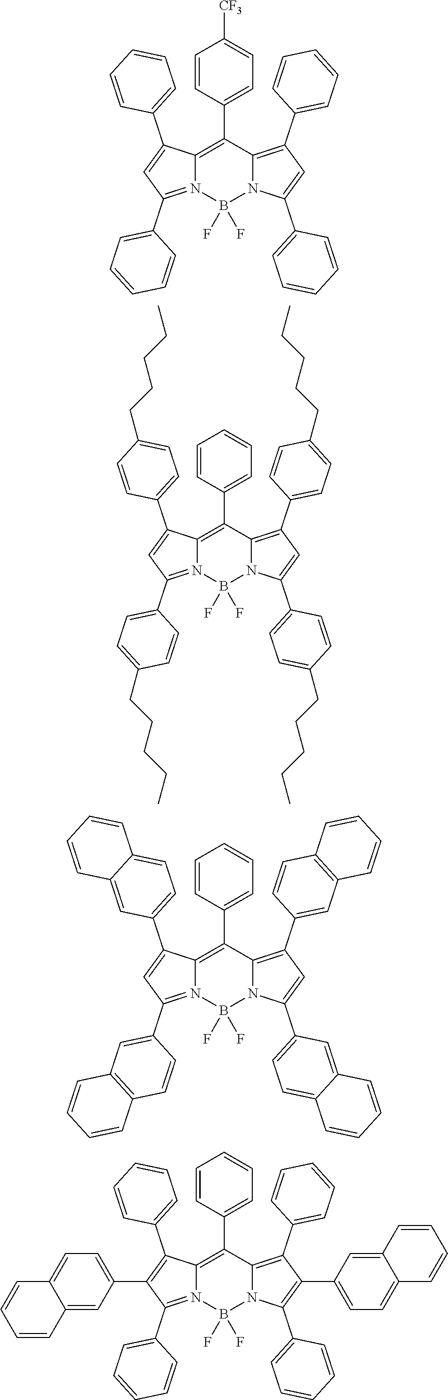

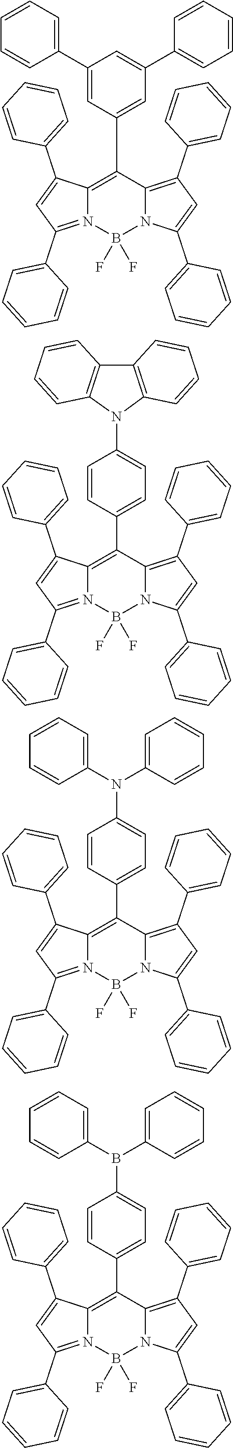

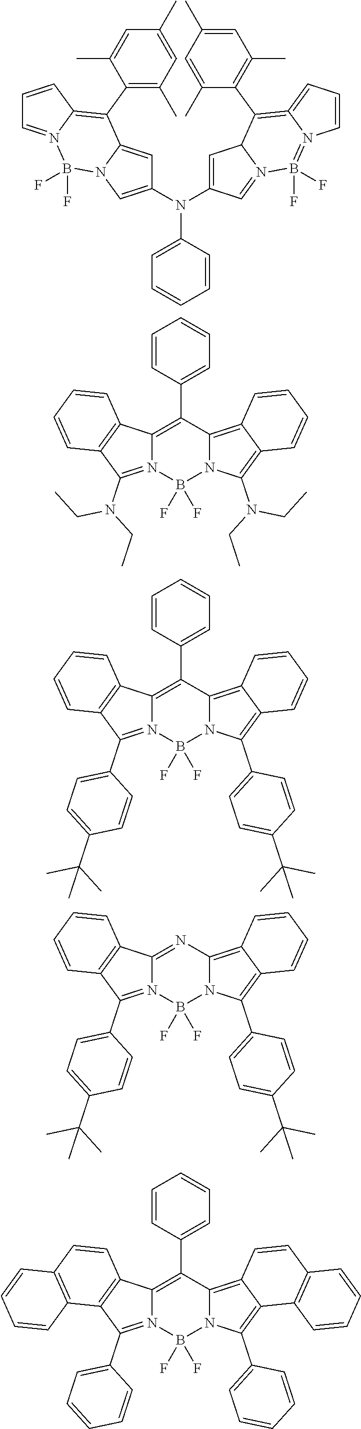

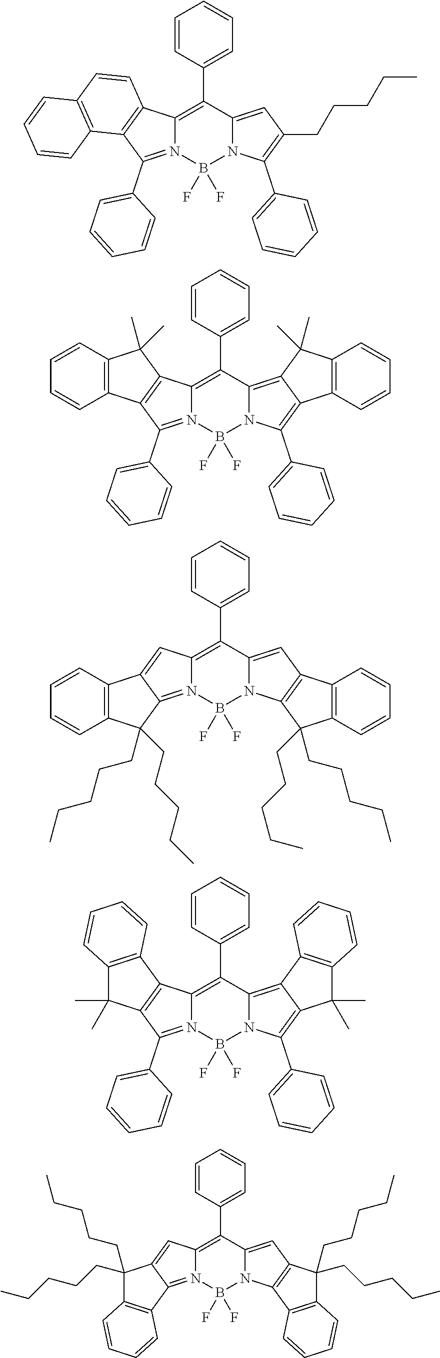

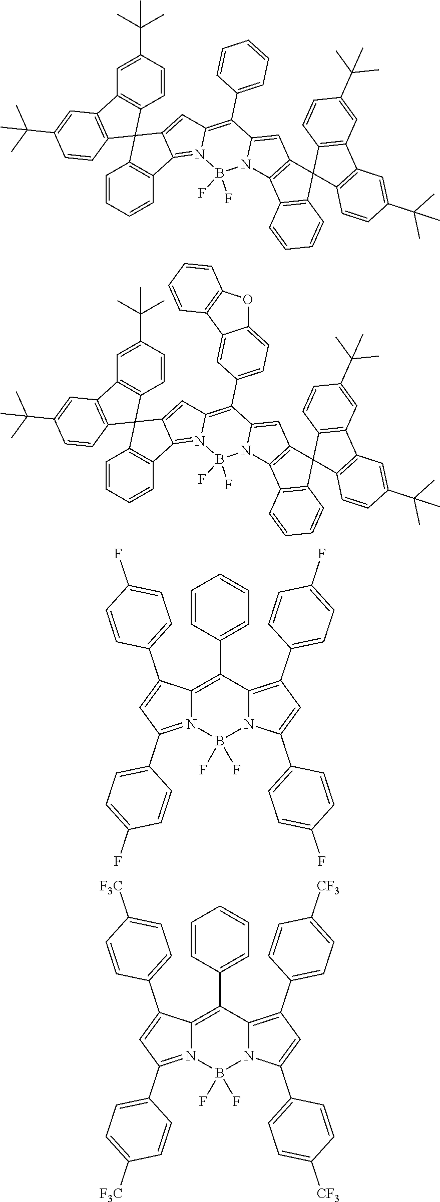

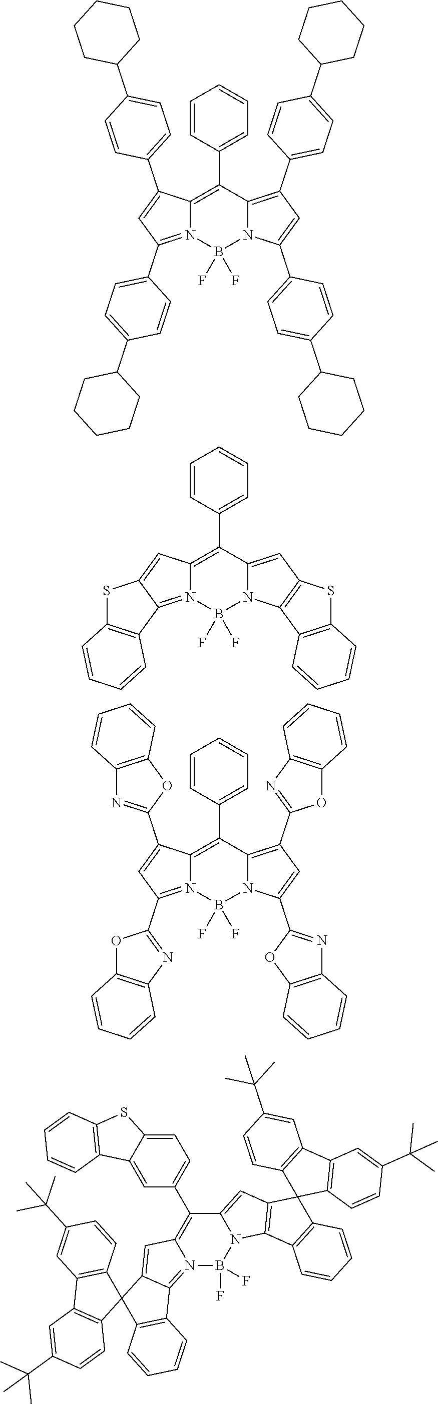

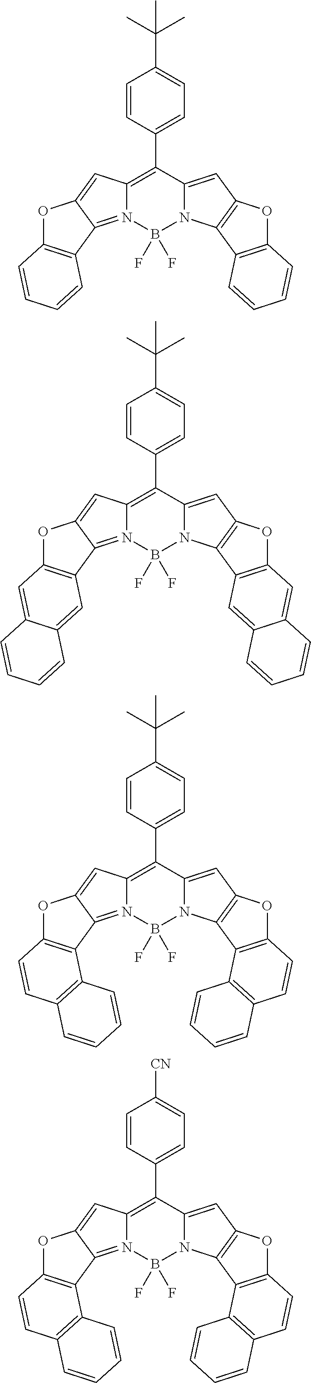

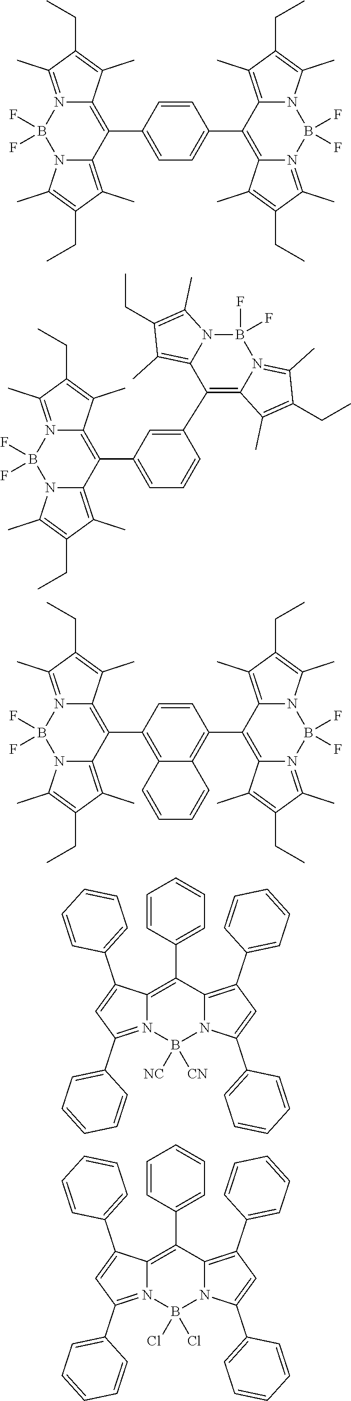

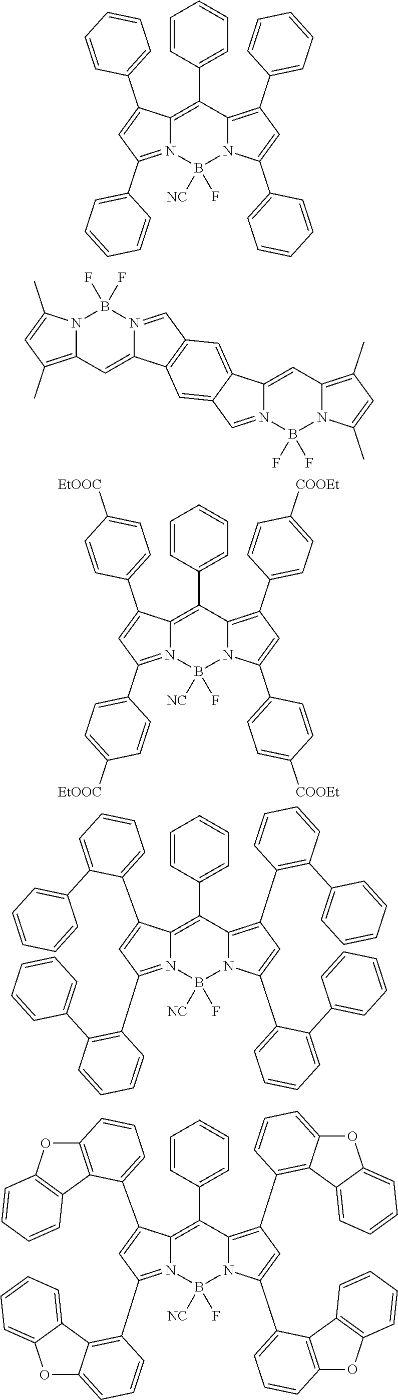

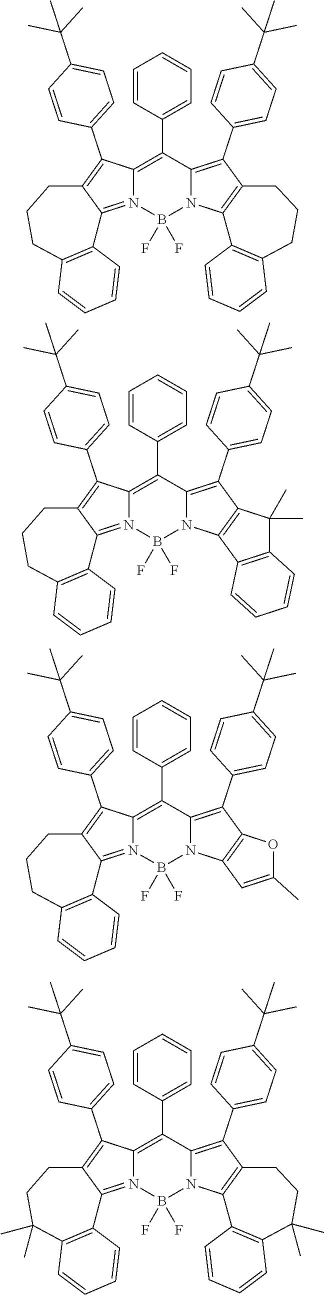

In some embodiments, in the organic compound H as described herein, at least one of R⁰¹—R¹⁰⁴ is selected from one of formulas (Ia-5)-(Ia-6):

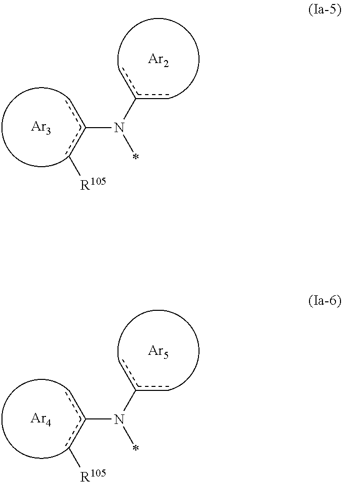

Where each Ar² is identically defined as described herein; each Ar³ is independently selected from an aromatic or heteroaromatic group containing 8 to 24 ring atoms, which may be further substituted; each Ar⁴ or Ar⁵ is independently selected from an aromatic or heteroaromatic group containing 5 to 24 ring atoms, and formula (Ia-6) comprises an electron withdrawing group; each R¹⁰⁵ is identically defined as above; each * independently represents an attachment site connecting a pyrene.

Preferably, Ar₄ or Ar₅ comprises an electron withdrawing group in formula (Ia-6), that is, Ar₄ or Ar₅ is an electron withdrawing group or substituted by an electron withdrawing group.

In some embodiments, the organic compound H comprises two electron withdrawing groups.

In some embodiments, the organic compound H comprises three electron withdrawing groups.

In some embodiments, the organic compound H comprises more than three electron withdrawing groups.

The above-mentioned electron withdrawing group may be selected from F, a cyano group, or one of the following groups:

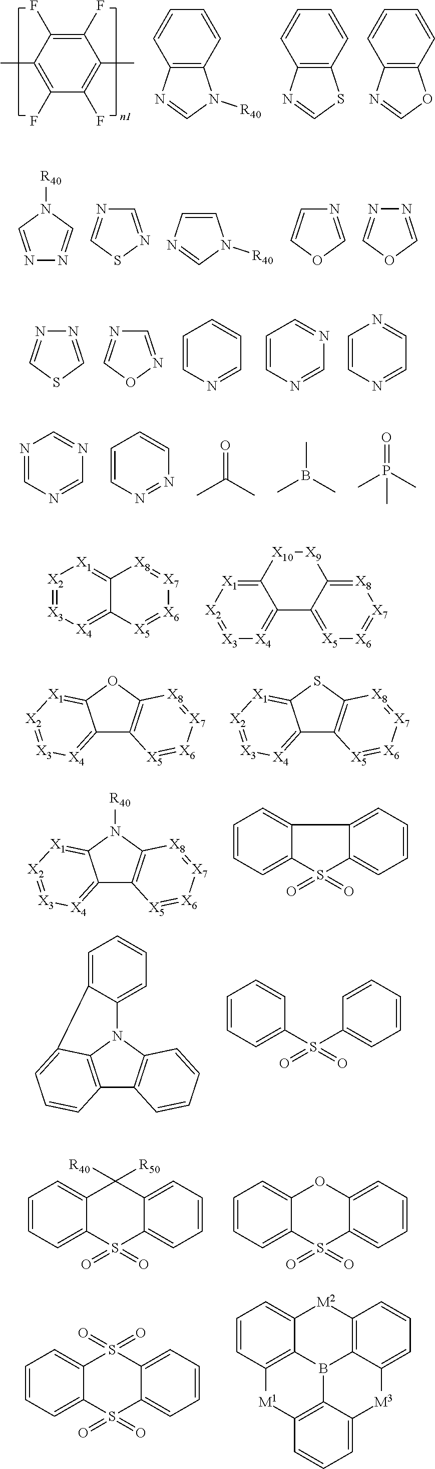

Where n₁ is an integer from 1 to 3; each of X₁ to X₁₀ is independently CR⁶⁰ or N, and at least one of them is N, but two adjacent Xs cannot be N at the same time; M¹, M², and M³ independently represent N(R⁶⁰), C(R⁶⁰R⁷⁰)₂, Si(R⁶⁰R⁷⁰)₂, O, C═N(R⁶⁰), C═C(R⁶⁰R⁷⁰)₂, P(R⁶⁰) P(═O)R⁶⁰, S, S=O, SO₂, or null; R⁴⁰, R⁵⁰, R⁶⁰, and R⁷⁰ are identically defined as the above-mentioned R¹.

In some embodiments, suitable electron-withdrawing groups include, but not limited to, F, Cl, a cyano group, a partial/perfluorinated alkyl chain, or one of the following groups:

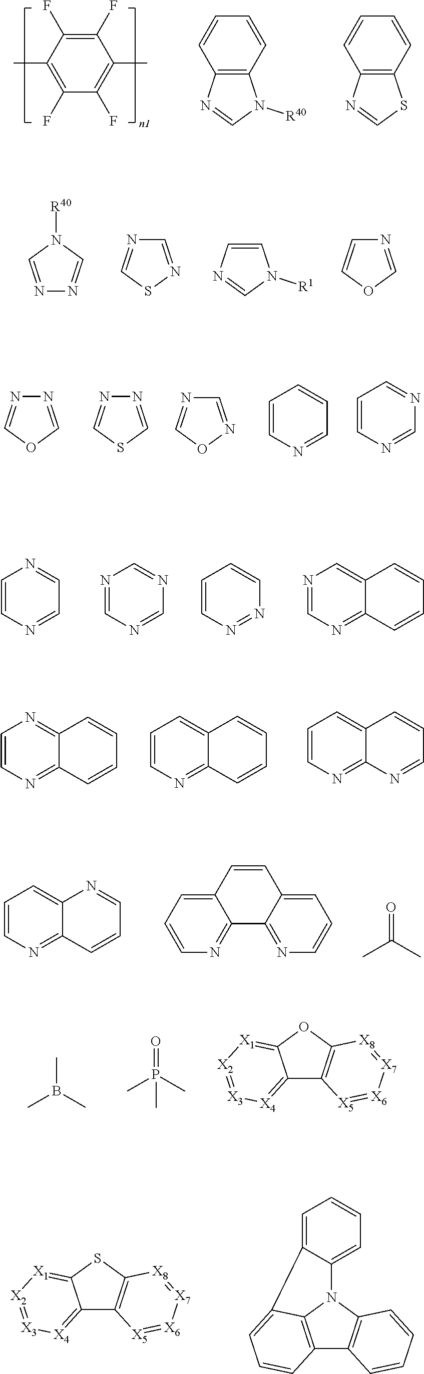

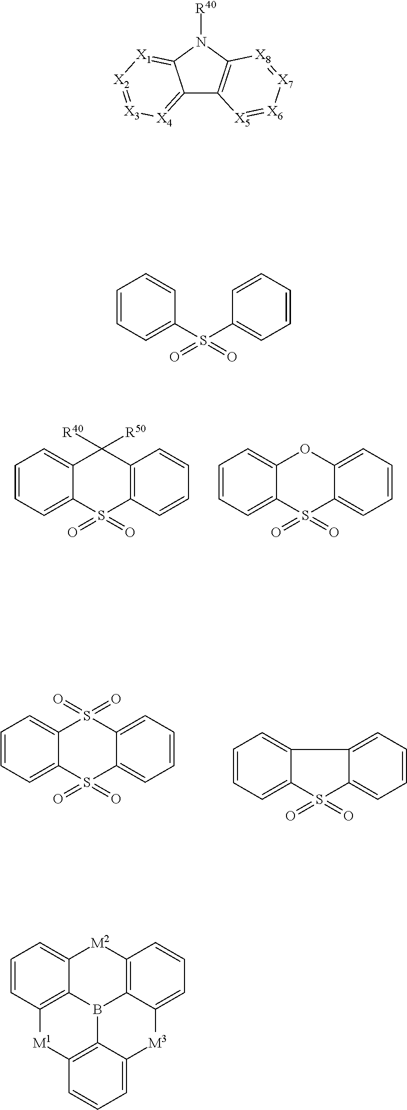

Where the symbols are identically defined as described herein.

In some embodiments, the organic compound H comprises F.

In some embodiments, the organic compound H comprises CN.

In some embodiments, the organic compound H comprises one of the following groups:

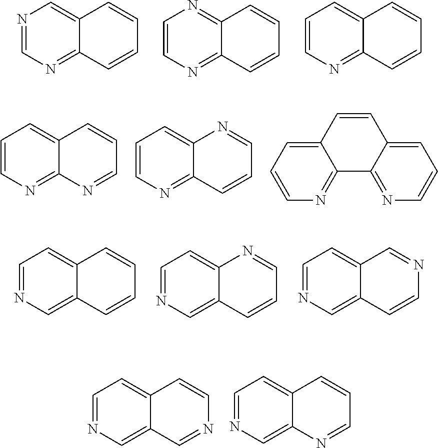

In some embodiments, Ar³ in formula (Ia-5) is selected from the following groups, which may be further substituted:

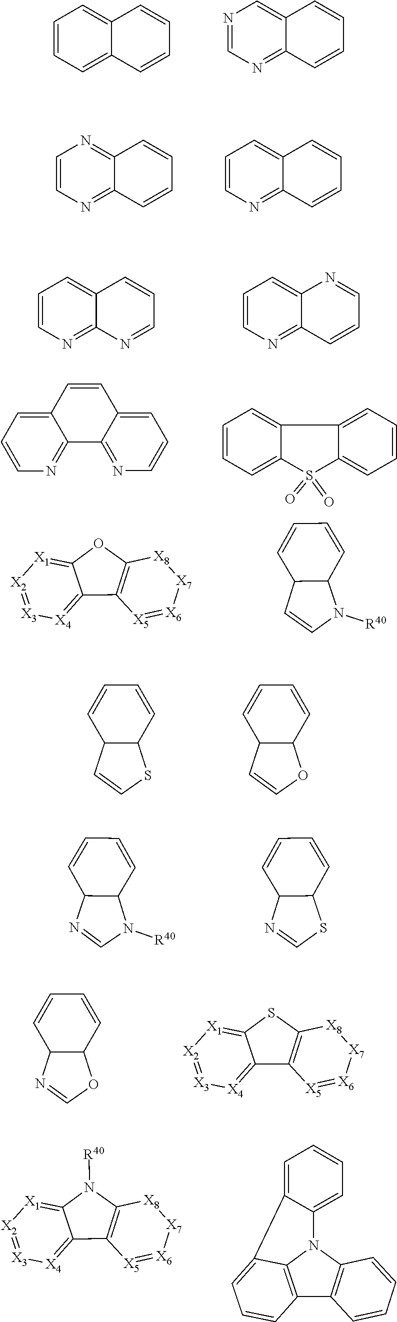

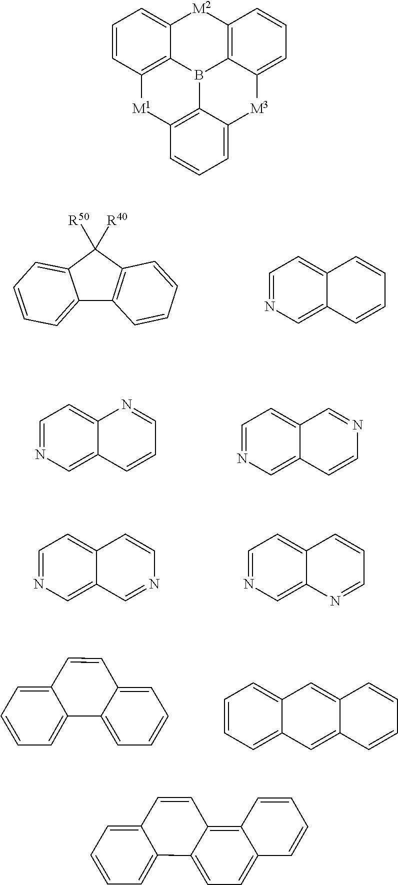

Where the symbols are identically defined as described herein.

In some embodiments, in organic compound H as described herein, at least two of R¹⁰¹-R¹⁰⁴ are each independently selected from one of formulas (Ia-1)-(Ia-6).

In some embodiments, in organic compound H as described herein, at least three of R¹⁰¹-R¹⁰⁴ are each independently selected from one of formulae (Ia-1)-(Ia-6).

In some embodiments, in organic compound H as described herein, R¹⁰¹-R¹⁰⁴ are each independently selected from one of formulas (Ia-1)-(Ia-6).

In the formulation as described herein, the organic compound H has relatively high extinction coefficient. The extinction coefficient is also known as the molar extinction coefficient, which refers to the absorption coefficient at a concentration of 1 mol/L, and is represented by the symbol E, in unit of Lmol⁻¹cm⁻¹. The extinction coefficient (ε) preferably ≥1*10³; more preferably ≥1*10⁴; even more preferably ≥2*10⁴; further preferably ≥3*10⁴; particularly preferably ≥5*10⁴; and most preferably ≥1*10⁵. Preferably, the extinction coefficient refers to the extinction coefficient at the wavelength corresponding to the absorption peak.

In some embodiments, the absorption spectrum of the organic compound His between 380 nm and 500 nm.

In some embodiments, the emission spectrum of the organic compound H is between 460 nm and 510 nm.

In some embodiments, the wavelength of the emission peak of the organic compound H <500 nm.

In some embodiments, the emission spectrum of the organic compound H is between 500 nm and 580 nm.

The energy structure of the organic compound plays a key role on its optoelectronic performance and stability.

In some embodiments, the organic compound H has a large ΔHOMO and/or ΔLUMO, generally ≥0.30 eV, preferably ≥0.40 eV, more preferably ≥0.50 eV, further preferably ≥0.60 eV, and most preferably ≥0.70 eV; where ΔHOMO═HOMO-(HOMO-1), ΔLUMO=(LUMO+1)-LUMO.

In the disclosure, (HOMO-1) is defined as the energy level of the second highest occupied molecular orbital, (HOMO-2) is defined as the energy level of the third highest occupied molecular orbital, and so on. (LUMO+1) is defined as the energy level of the second lowest unoccupied molecular orbital, (LUMO+2) is defined as the energy level of the third lowest occupied molecular orbital, and so on.

In some embodiments, the organic compound H has relatively large oscillator strength f(S1) (n>1); f(S1) generally 0.10, preferably ≥0.20, more preferably 0.30, even more preferably 0.40, further preferably 0.50, and most preferably 0.60 eV. The oscillator strength f(S1) can be calculated by the following method.

In some embodiments, f(S1)≥0.70, preferably ≥0.80, more preferably ≥0.90, even more preferably ≥1.00, further preferably ≥1.2, and most preferably ≥1.6.

In some embodiments, the organic compound H has relatively low HOMO, generally ≤−4.6 eV, preferably <−4.7 eV, more preferably <−4.8 eV, further preferably <−4.9 eV, particularly preferably <−5.1 eV, and most preferably <−5.2 eV.

In some embodiments, the organic compound H has high solubility in the organic solvent. Preferably, the organic compound H typically has a solubility of ≥10 mg/mL in the toluene, preferably ≥20 mg/mL, more preferably ≥40 mg/mL, even more preferably ≥70 mg/mL, further preferably ≥100 mg/mL, and most preferably ≥150 mg/mL.

Examples of some suitable organic compounds H are listed below (but not limited thereto), which can be further arbitrarily substituted:

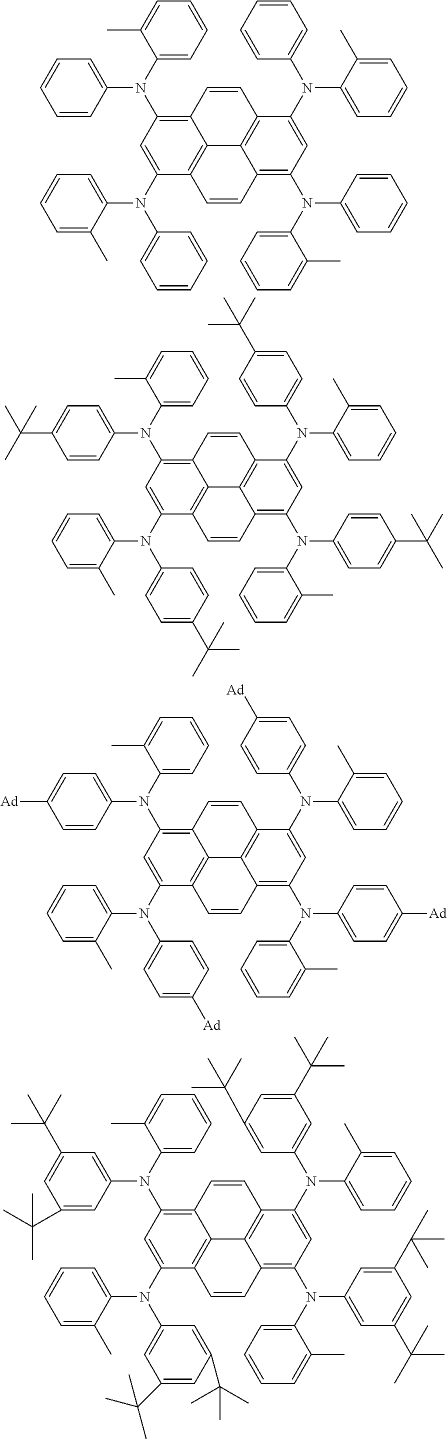

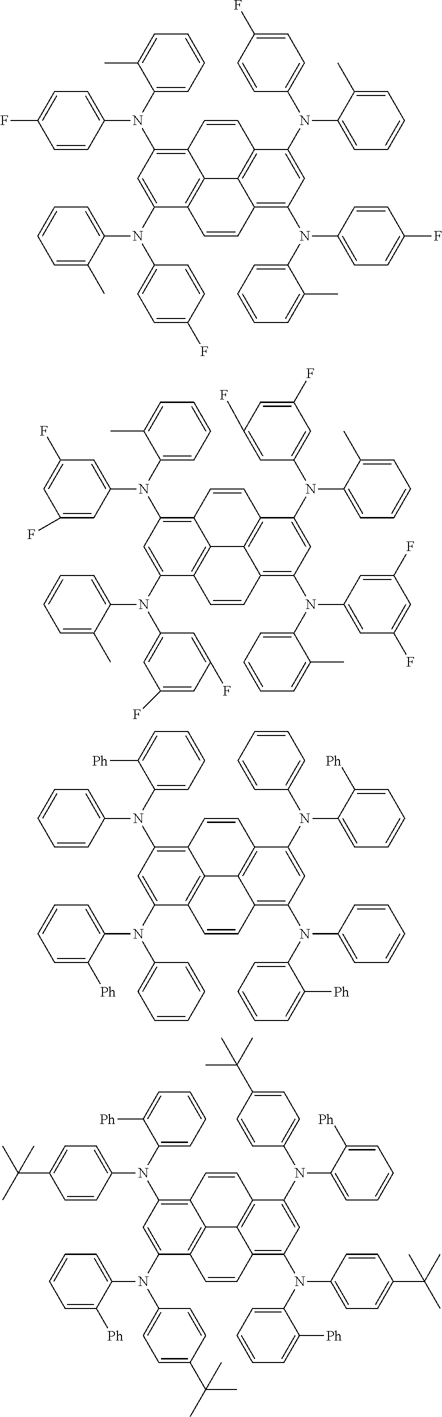

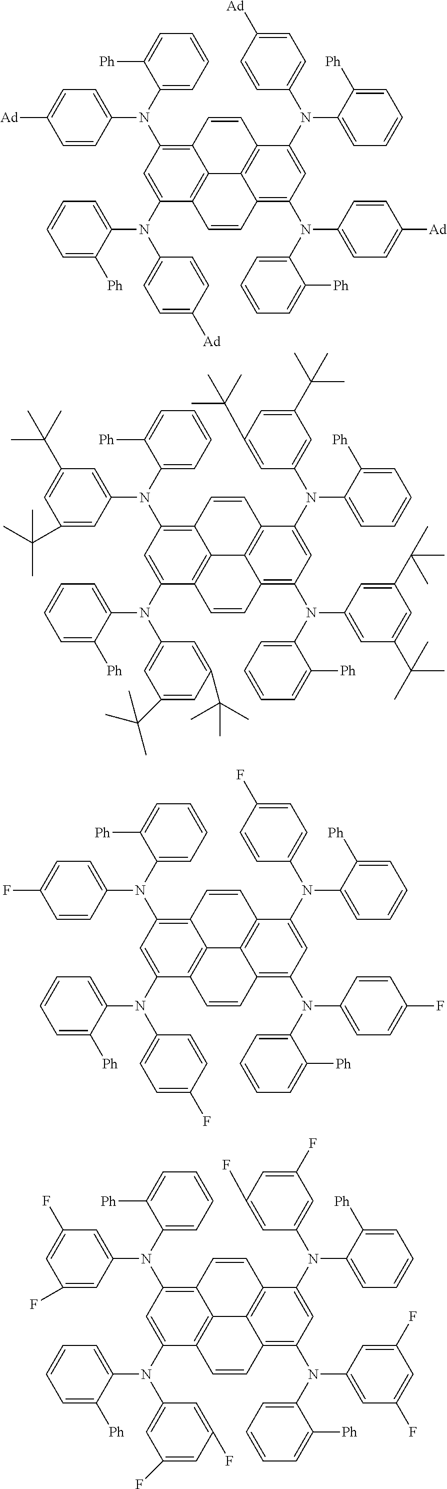

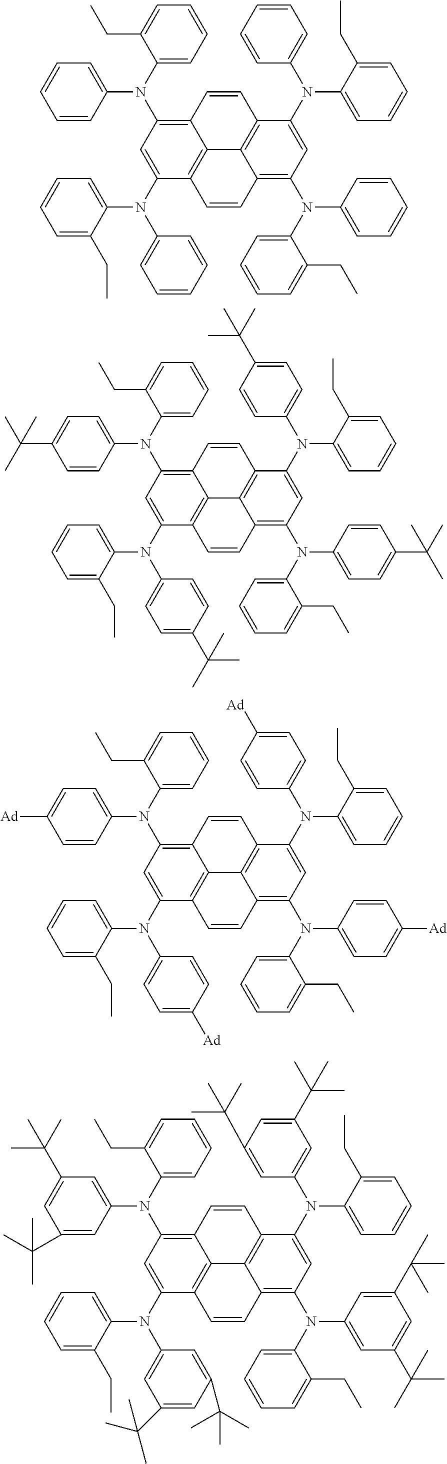

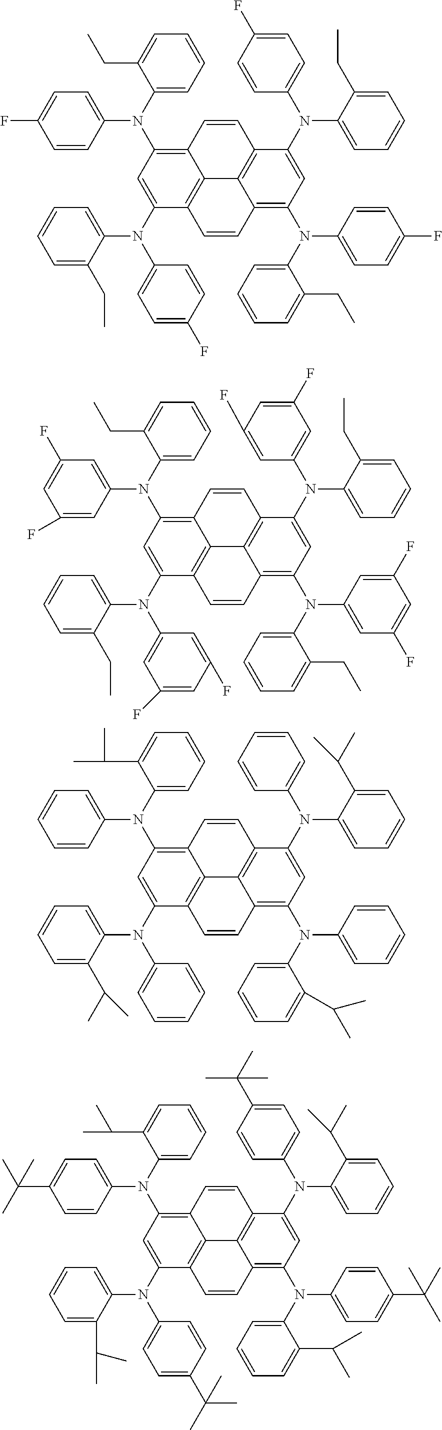

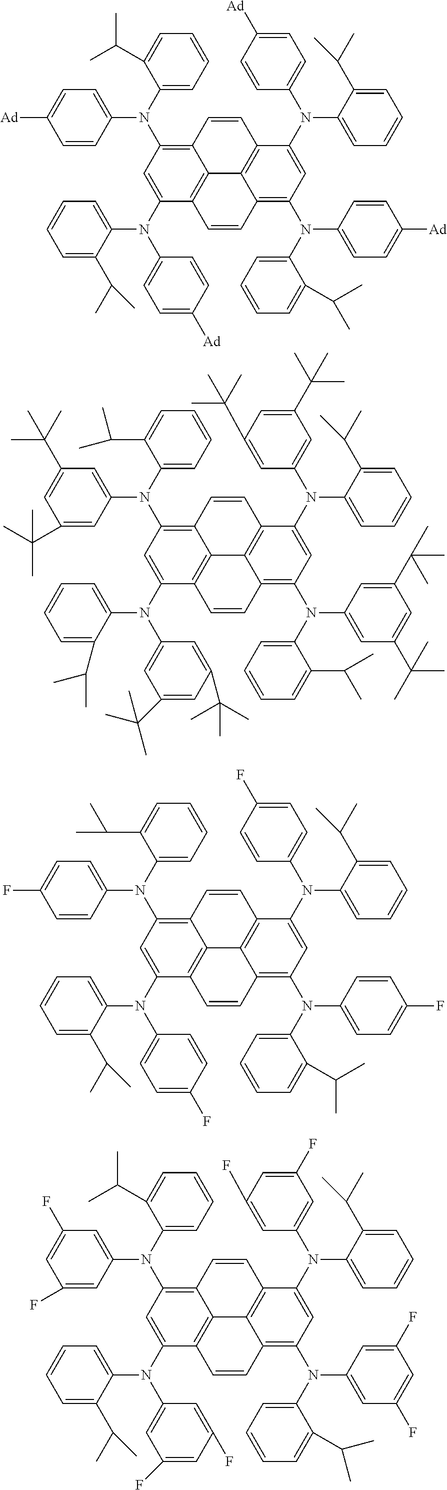

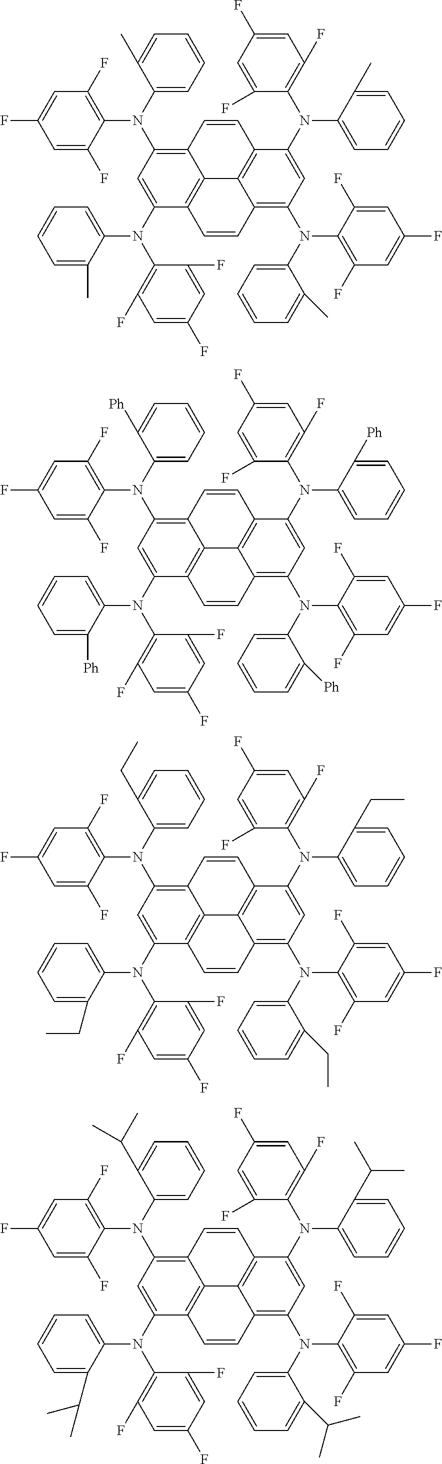

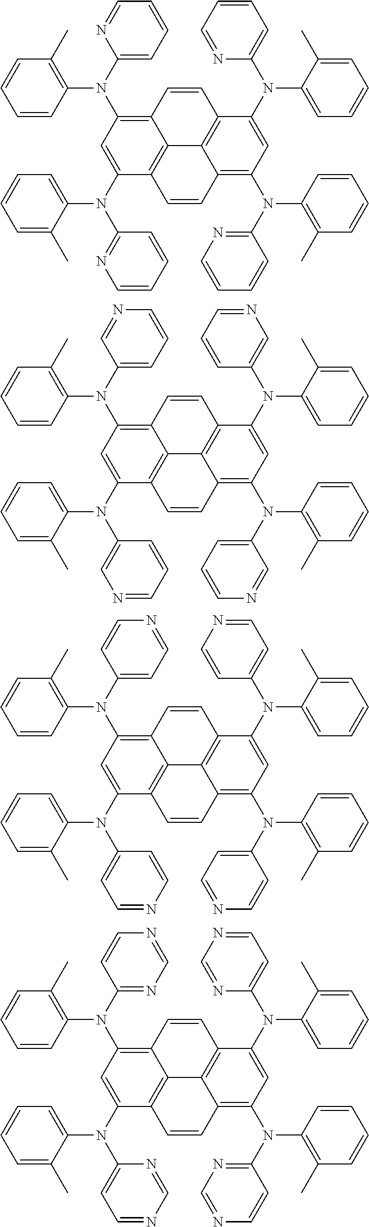

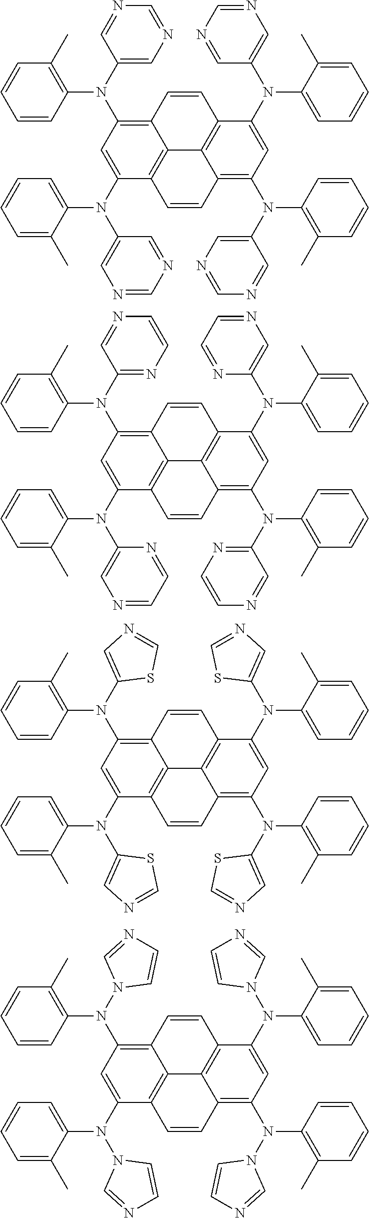

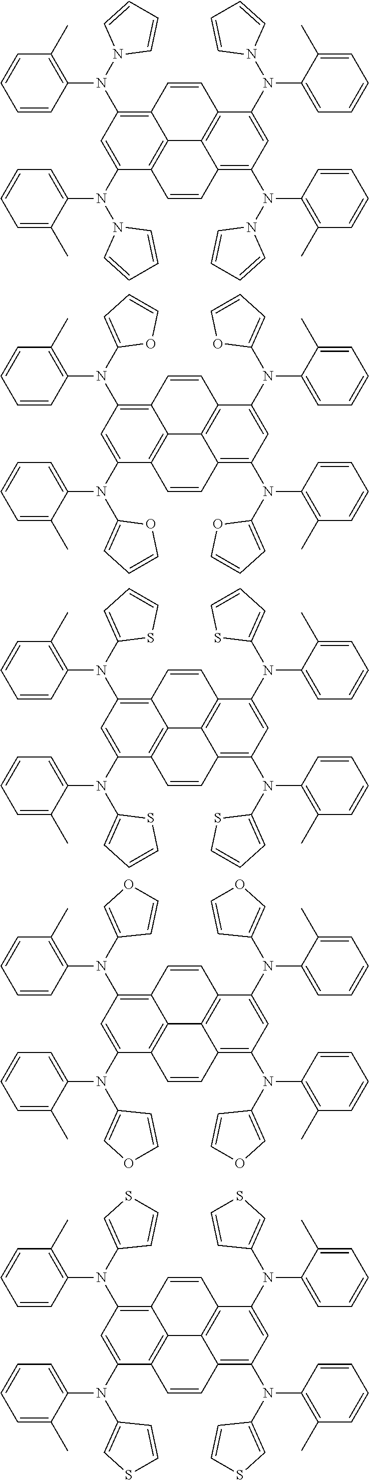

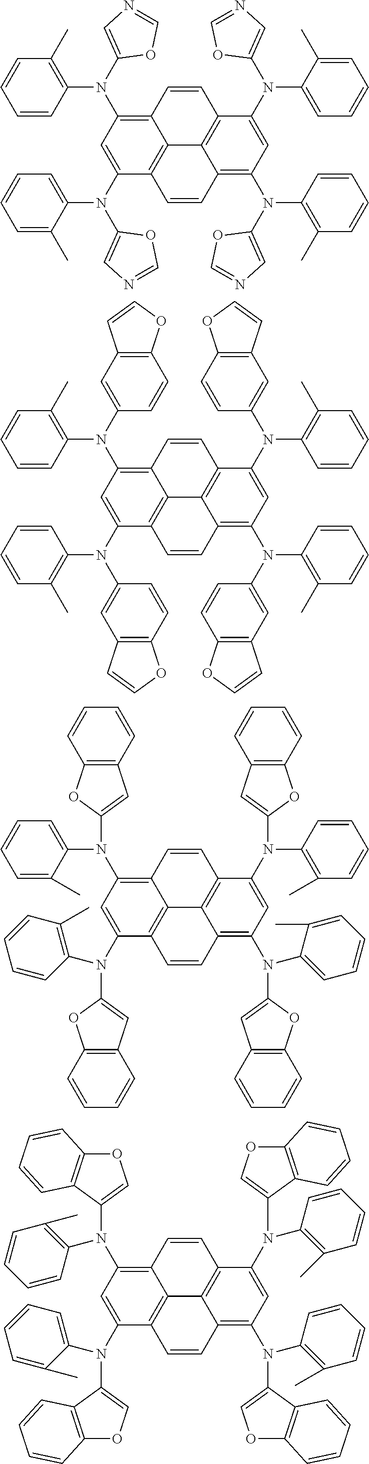

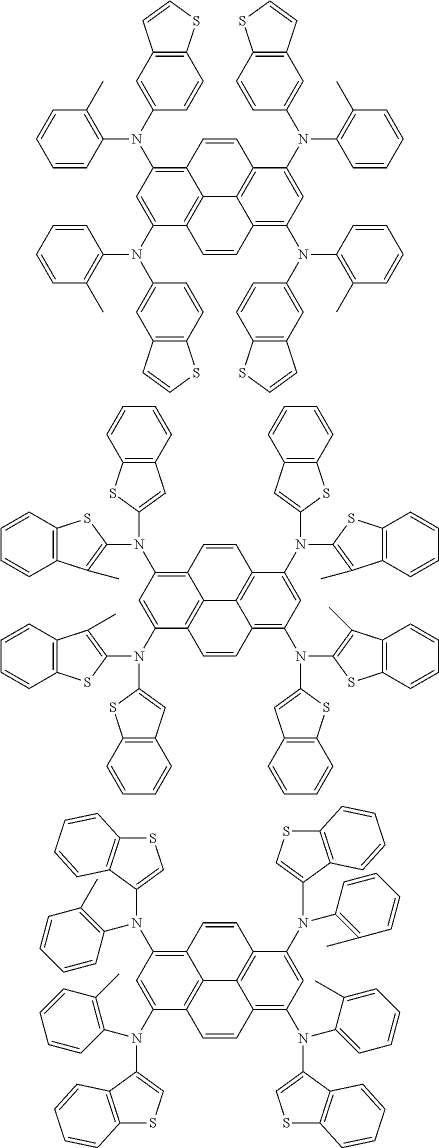

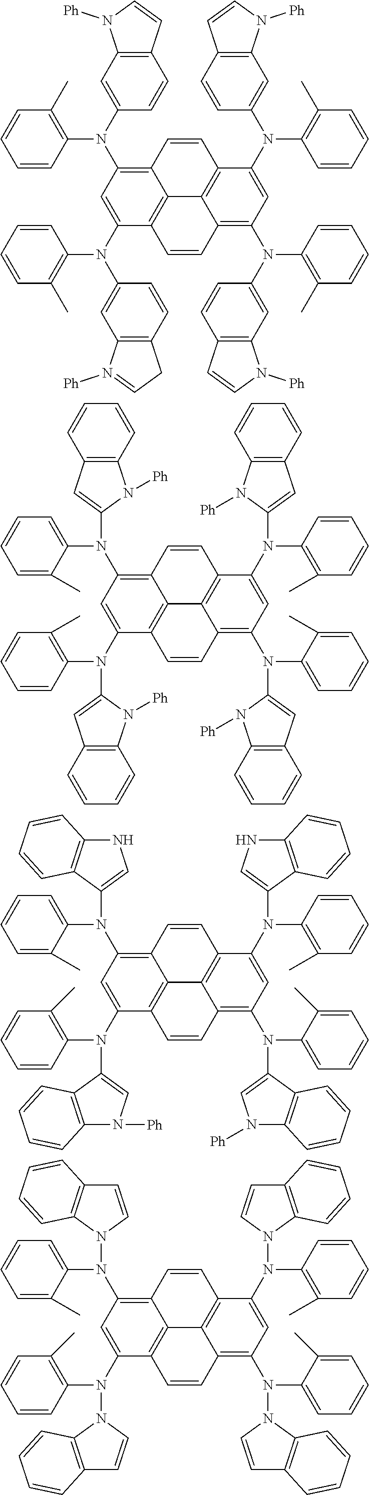

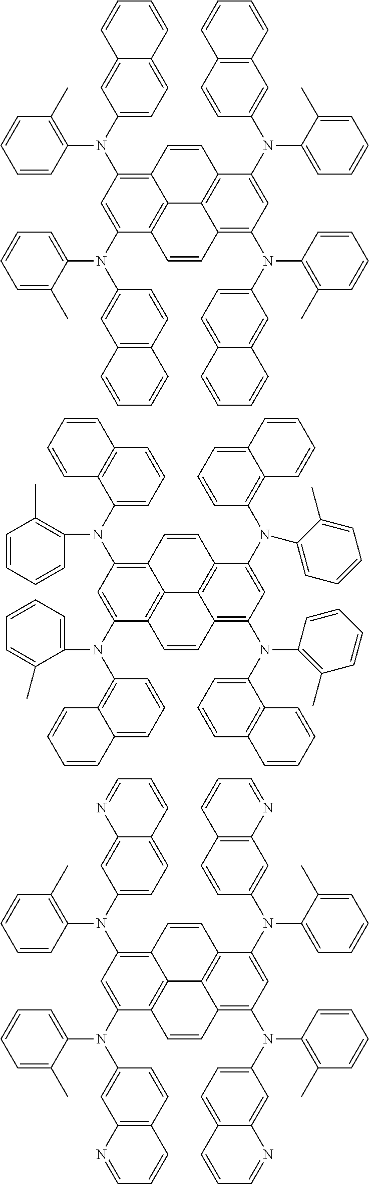

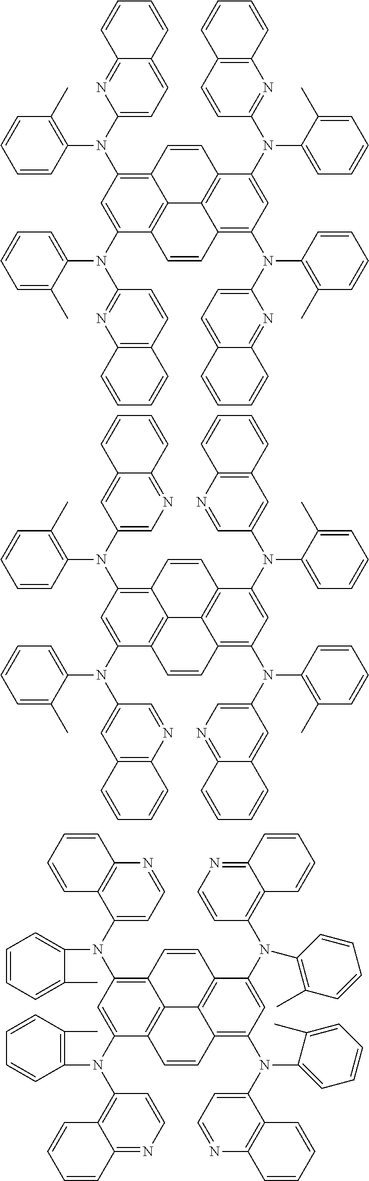

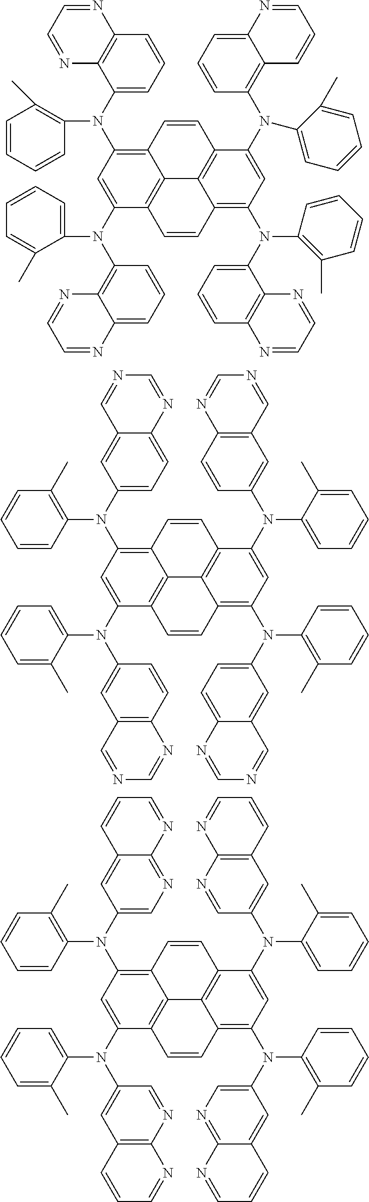

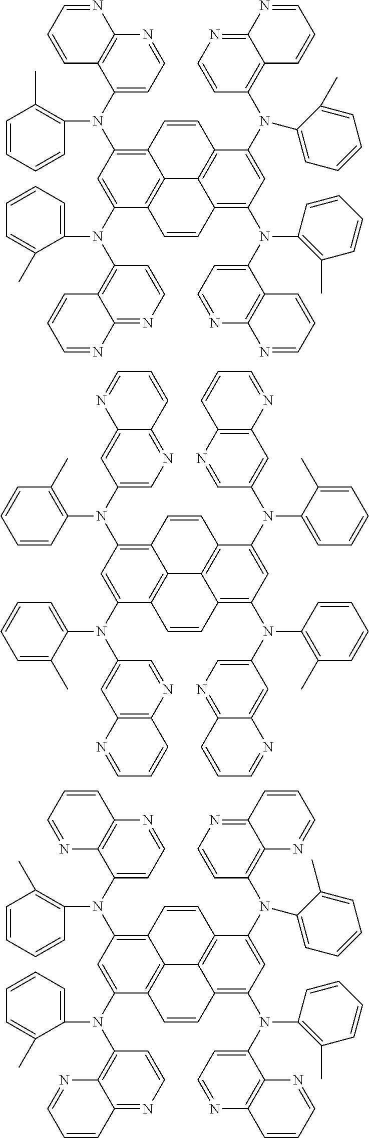

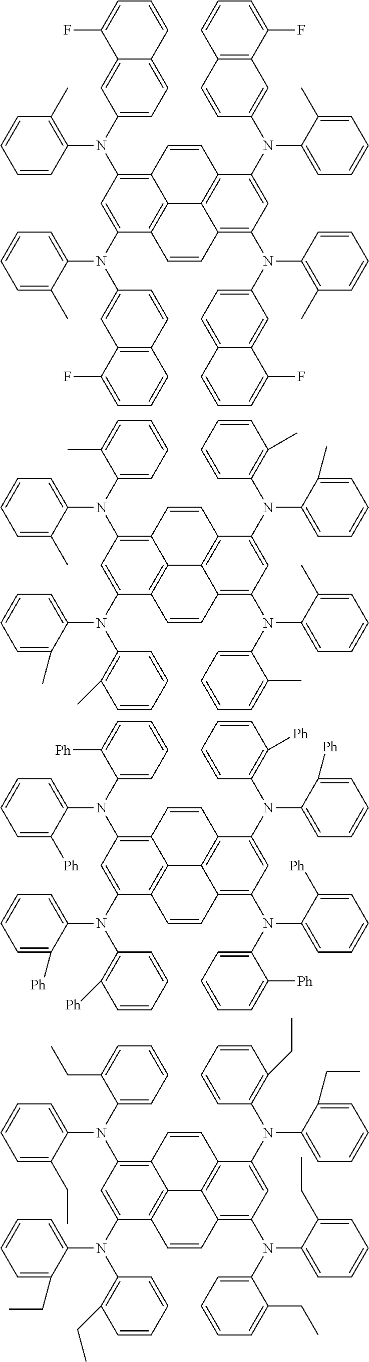

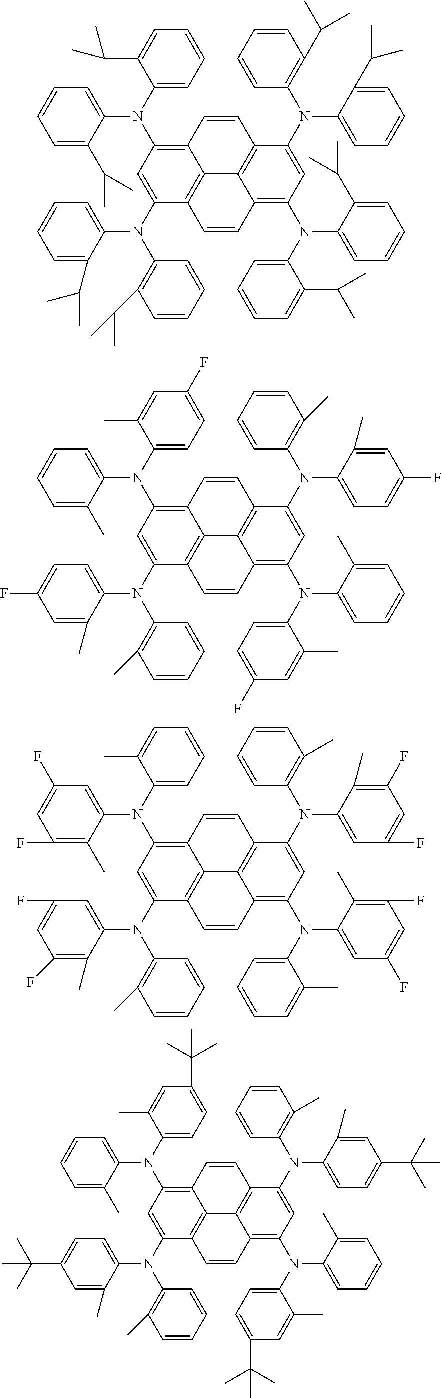

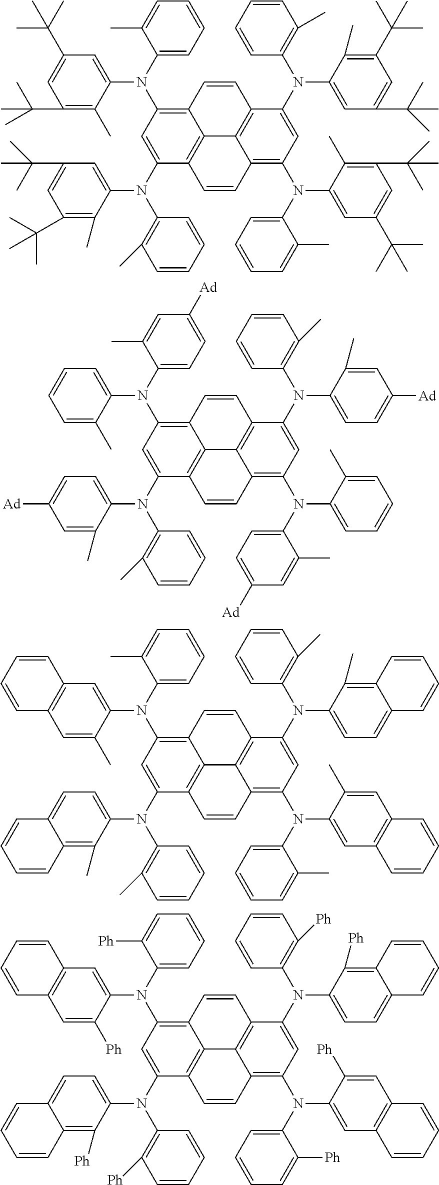

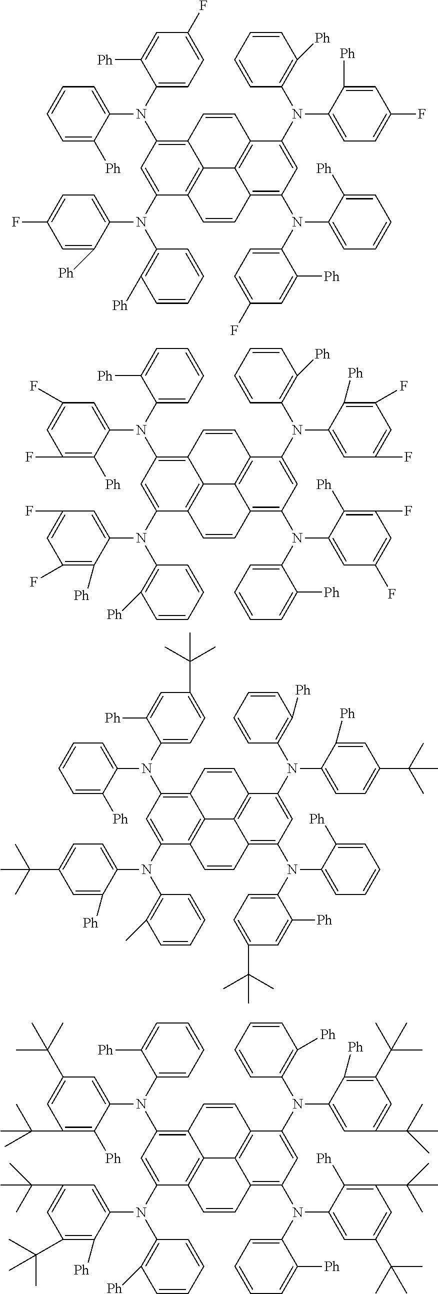

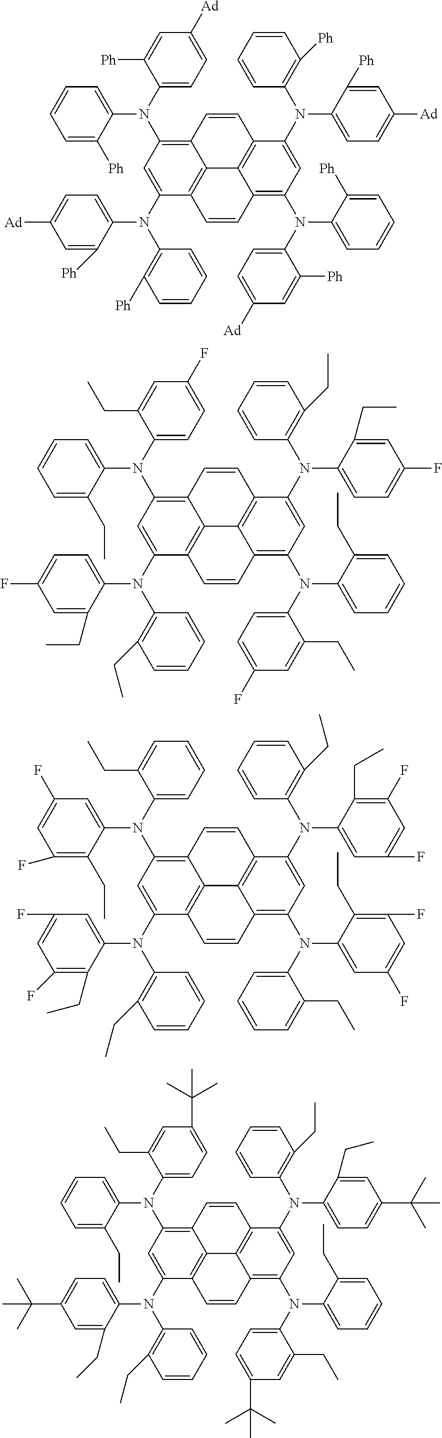

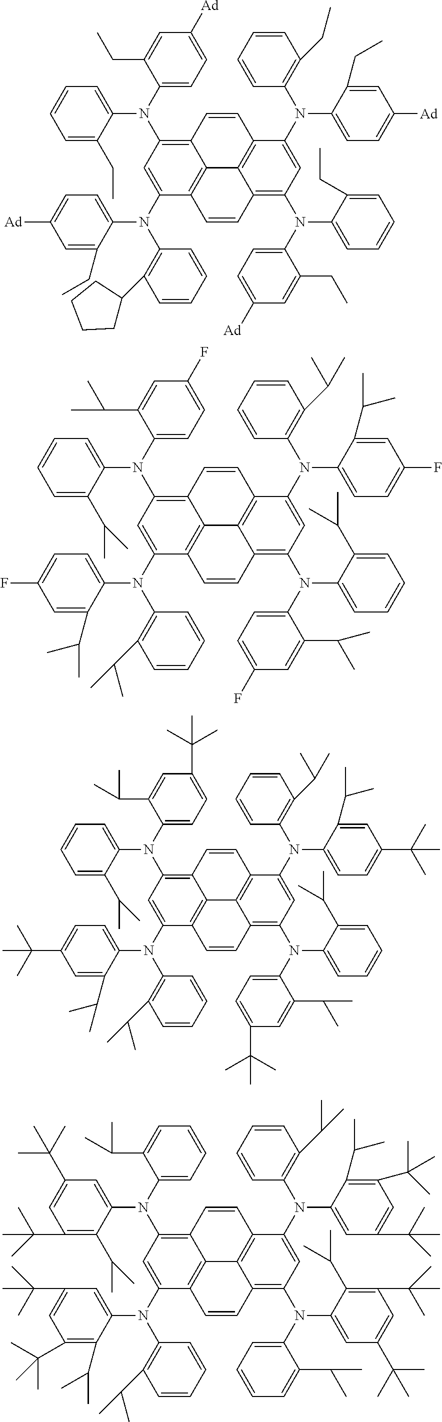

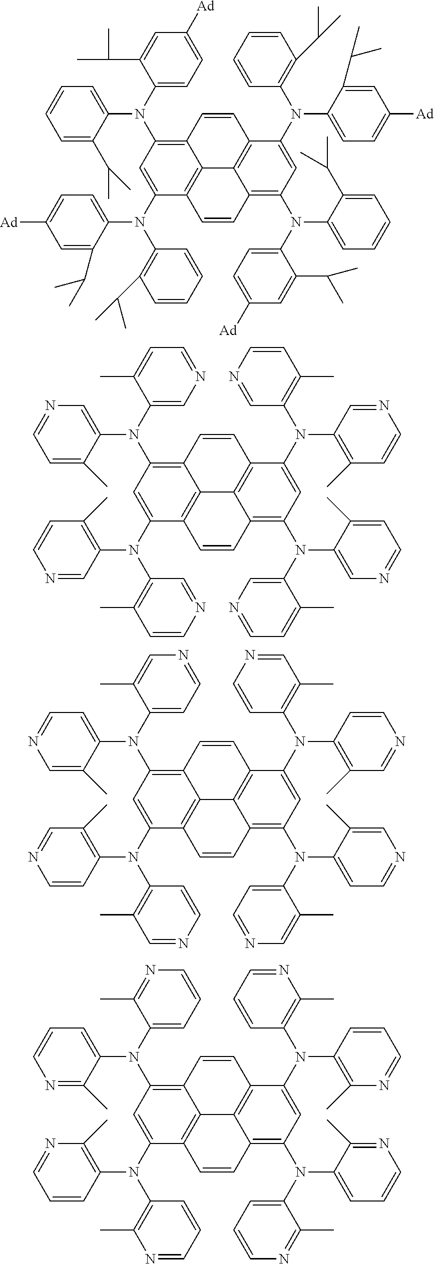

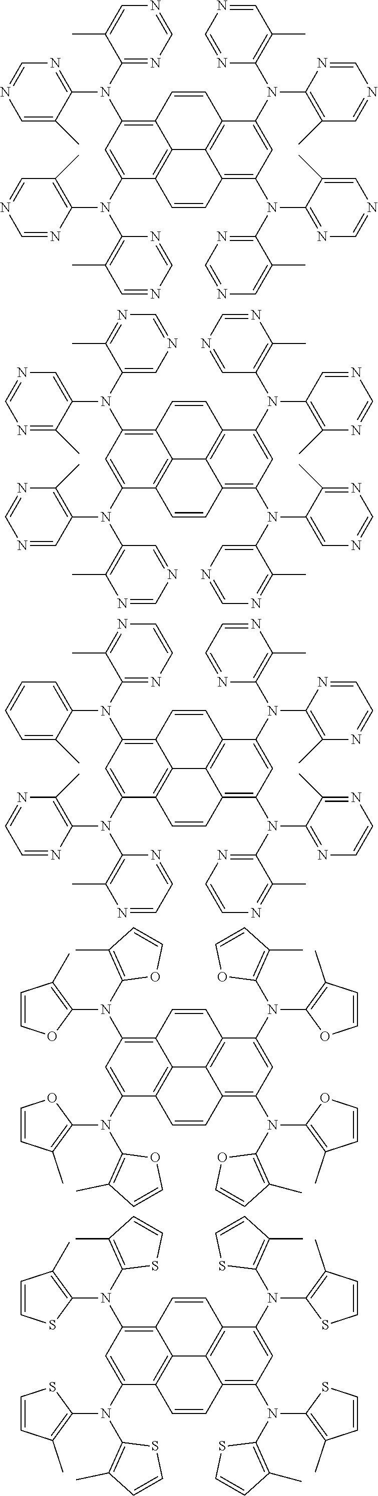

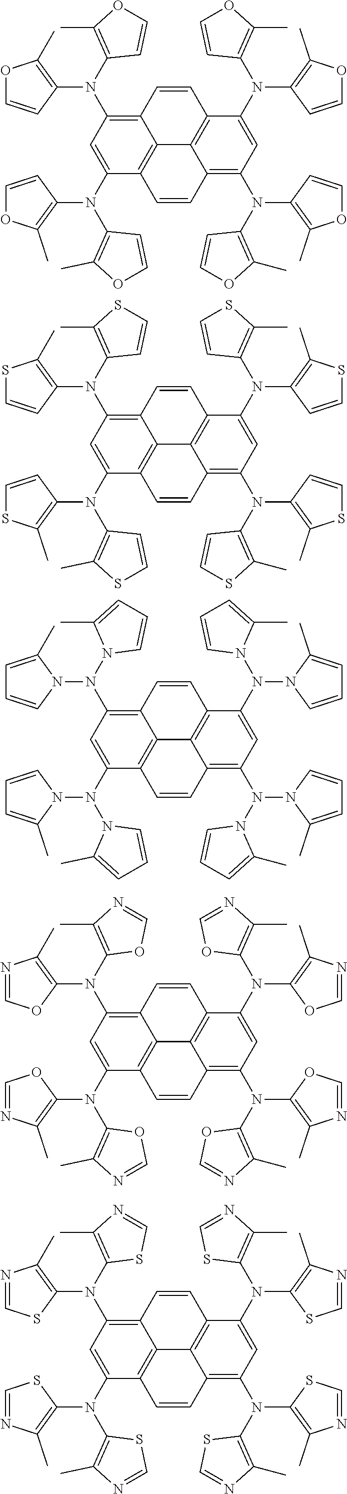

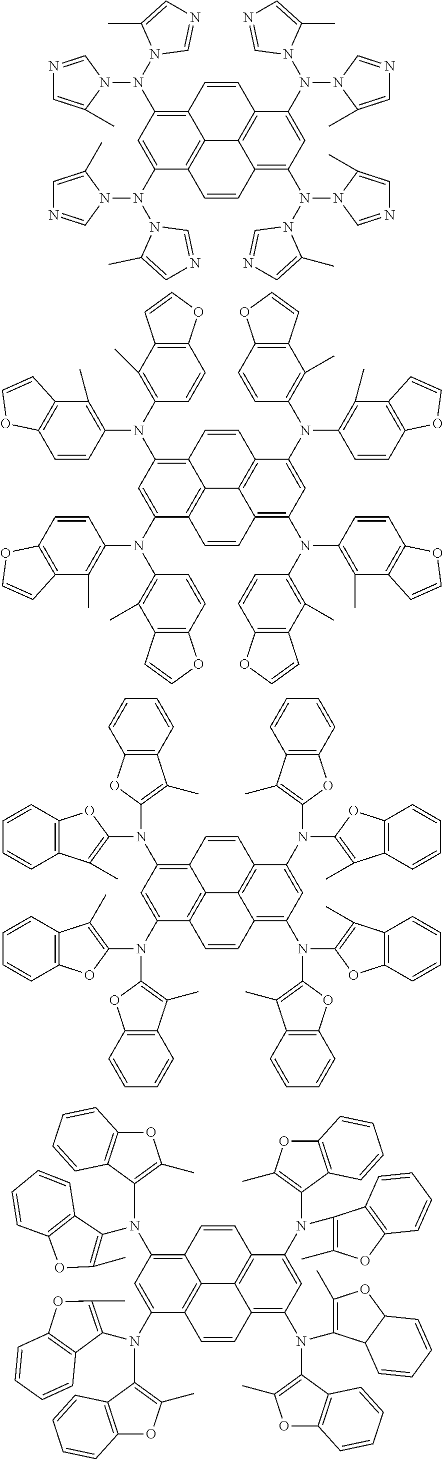

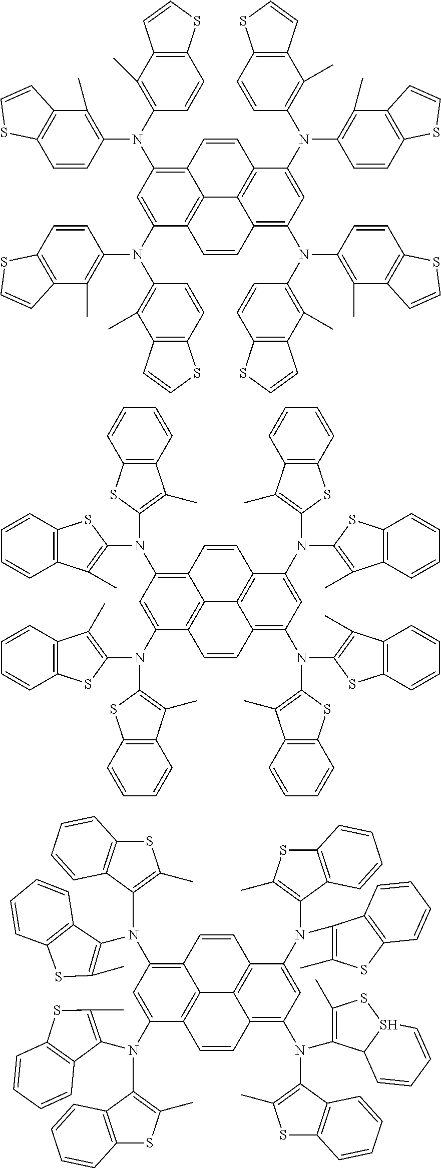

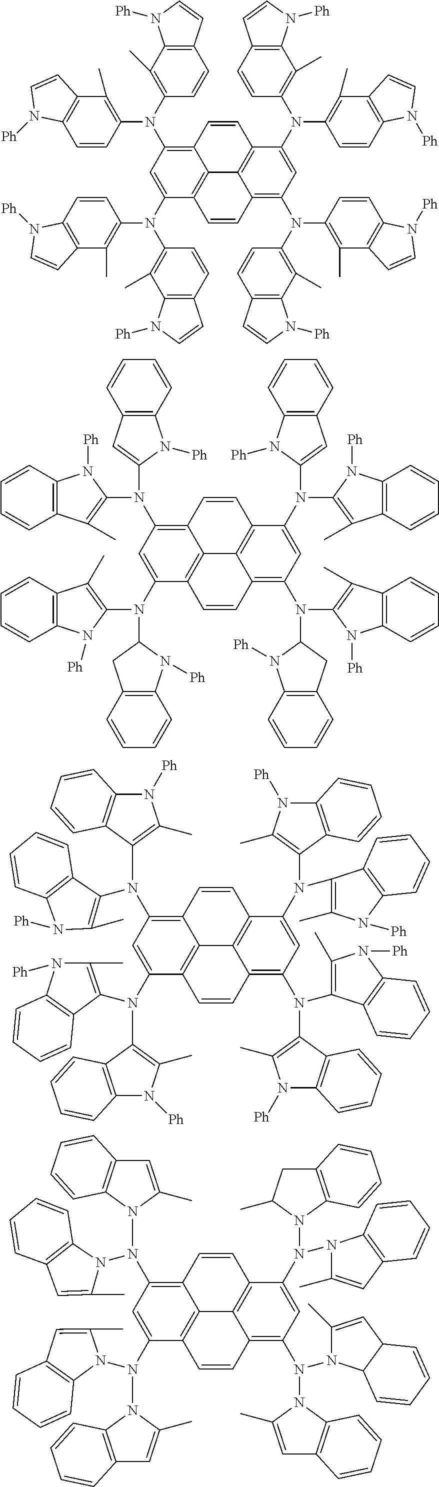

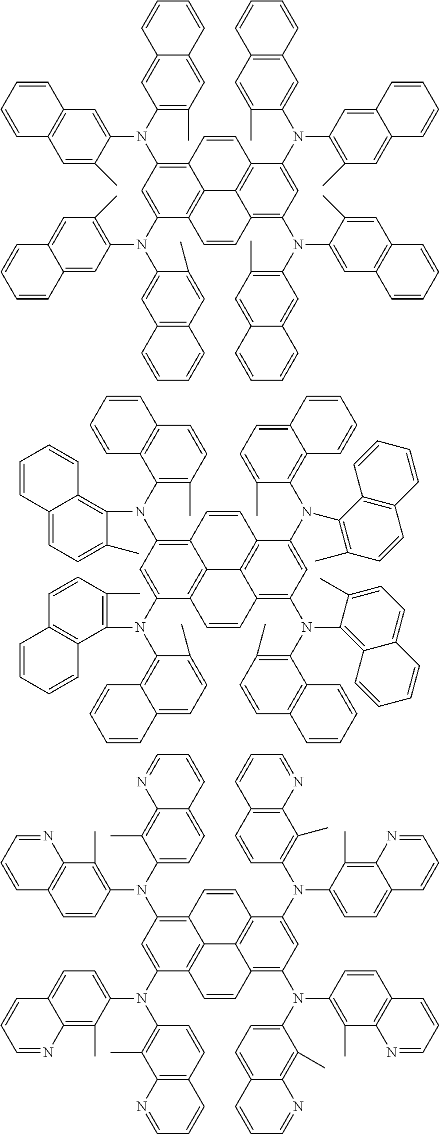

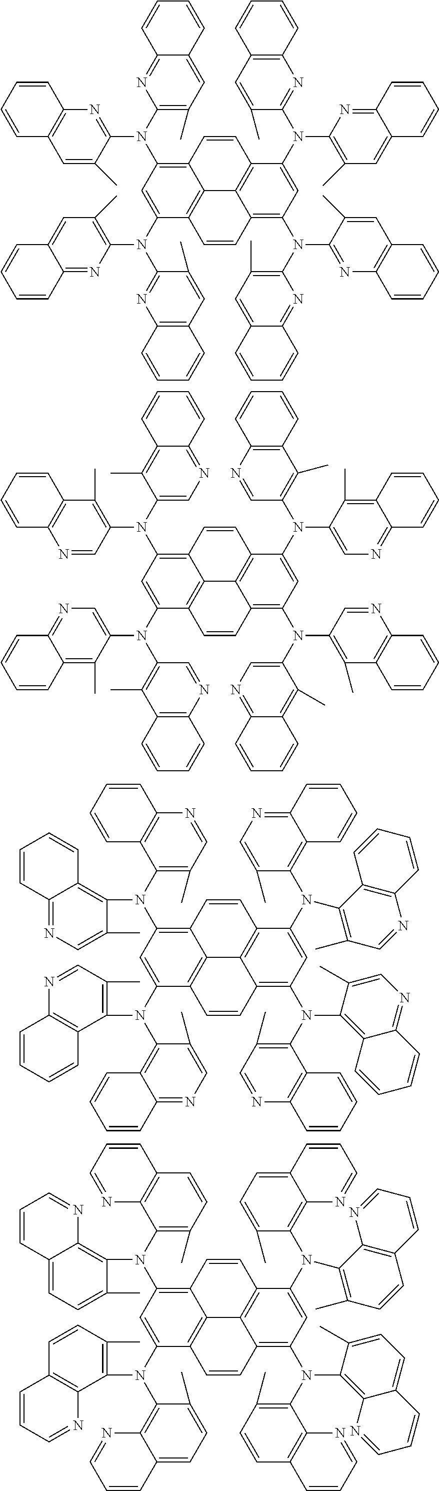

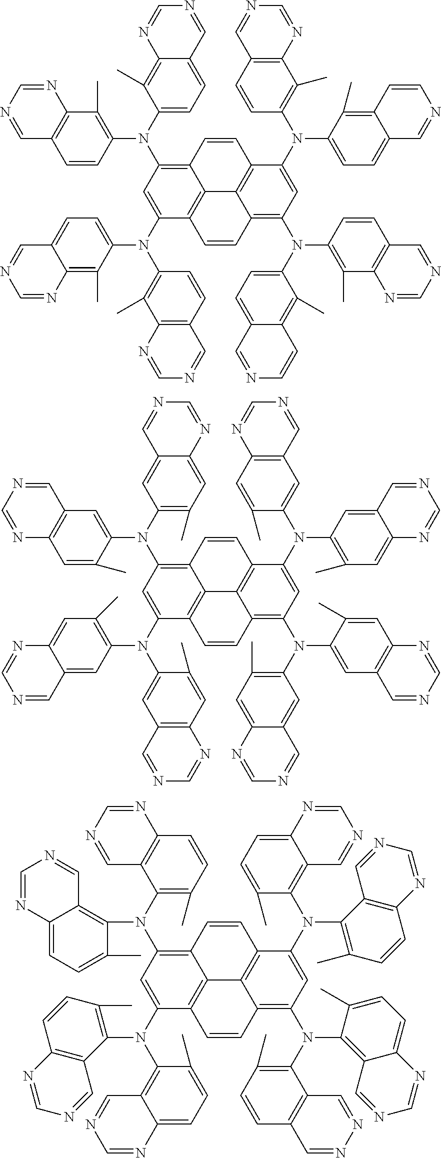

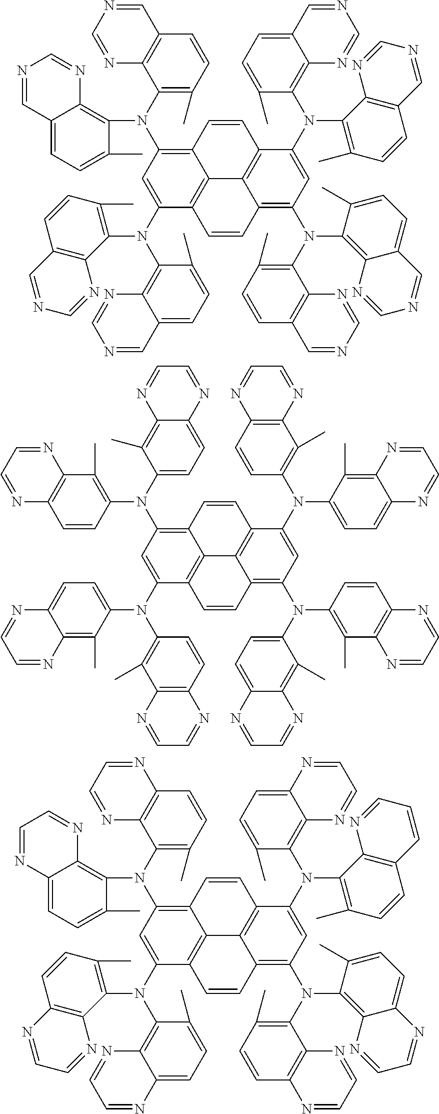

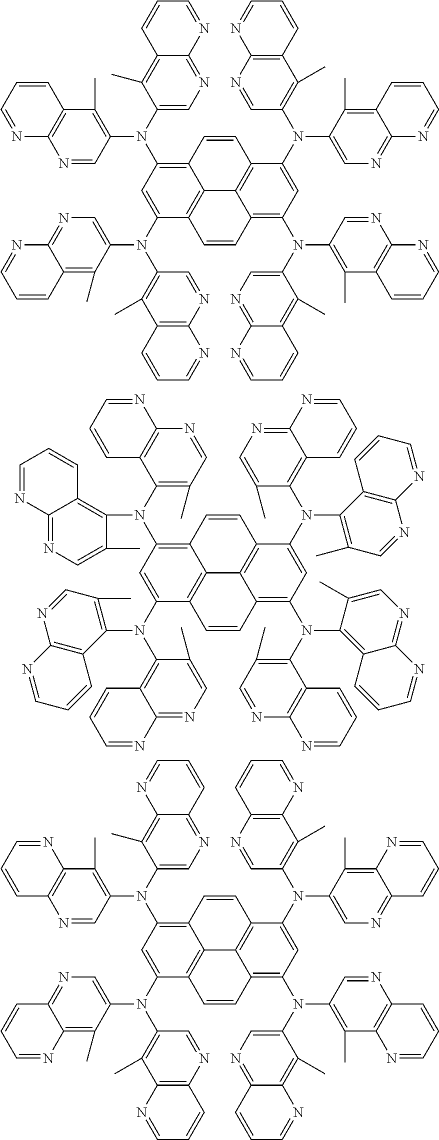

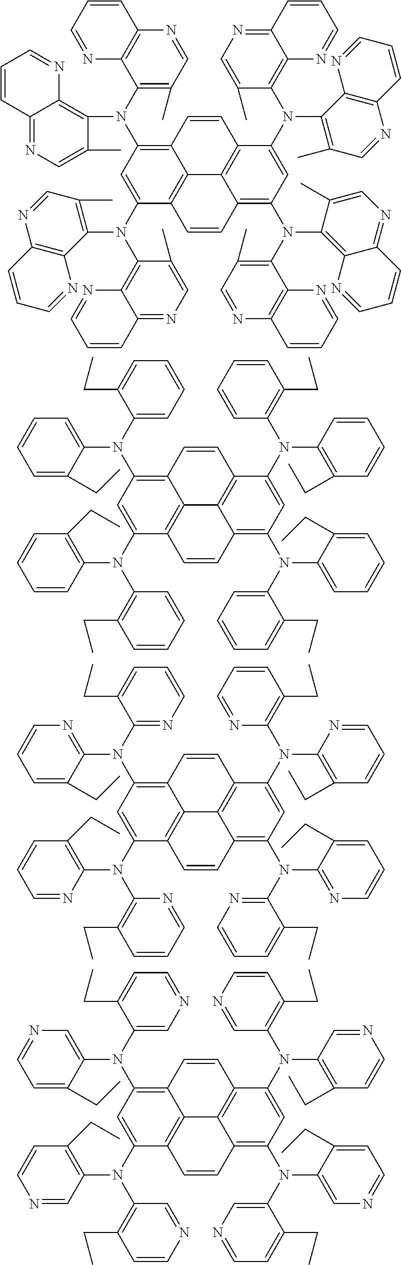

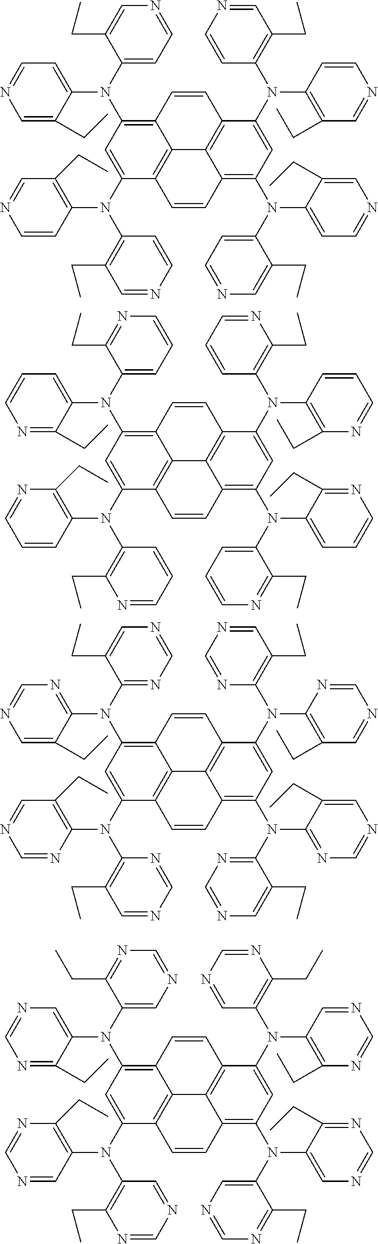

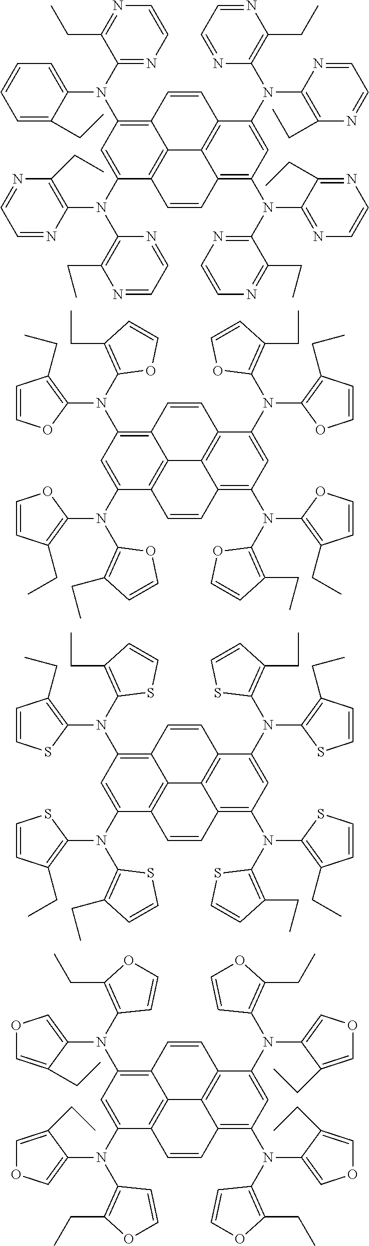

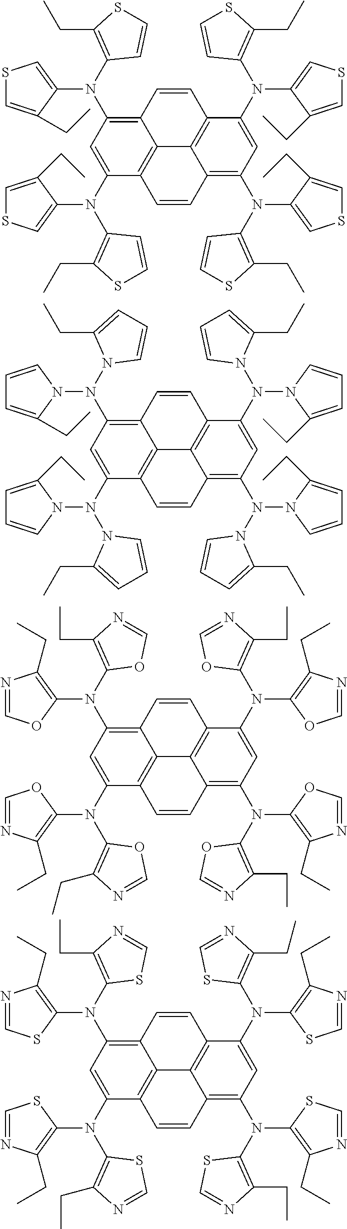

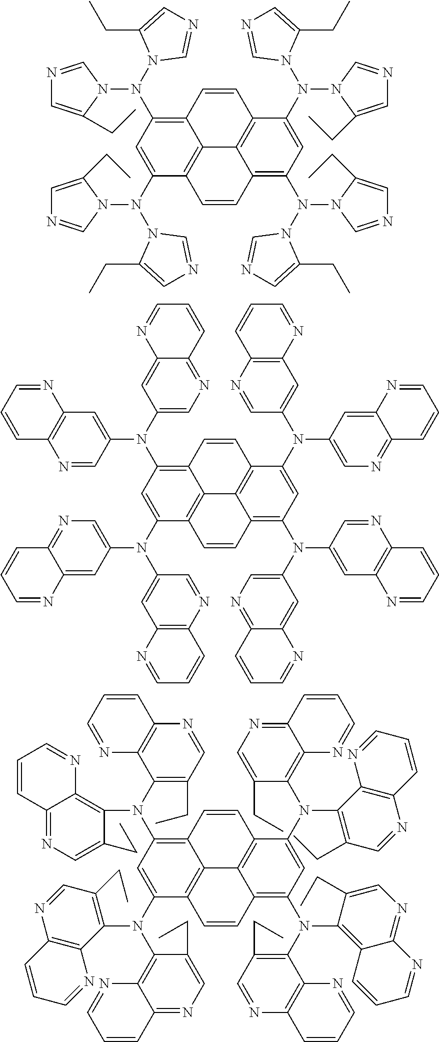

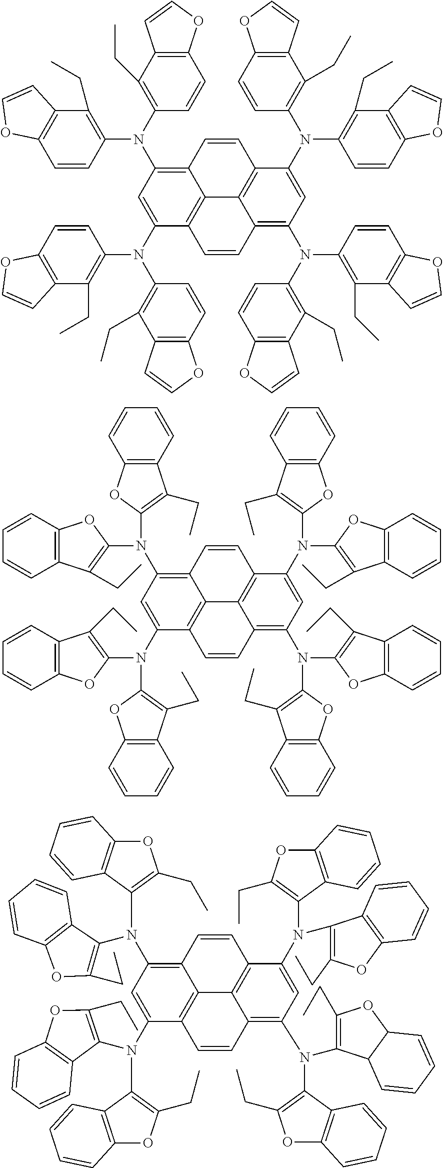

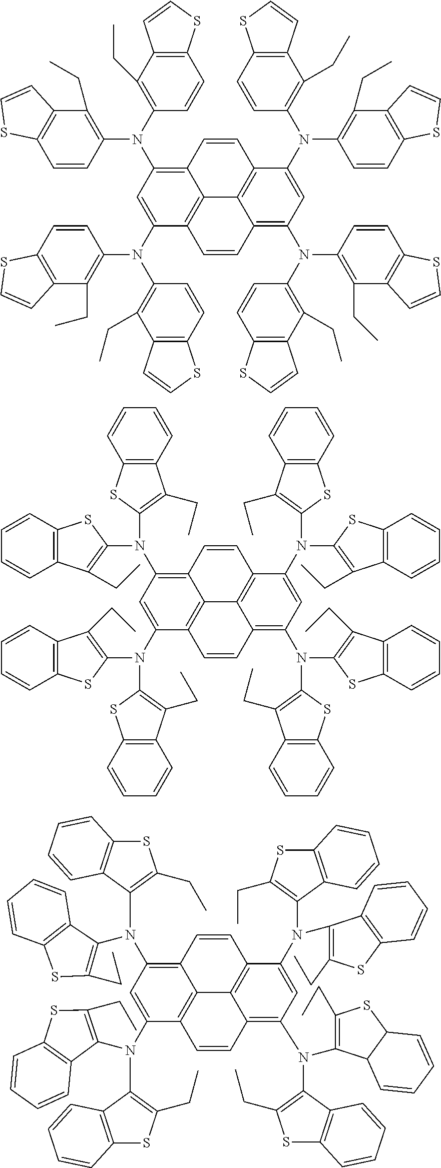

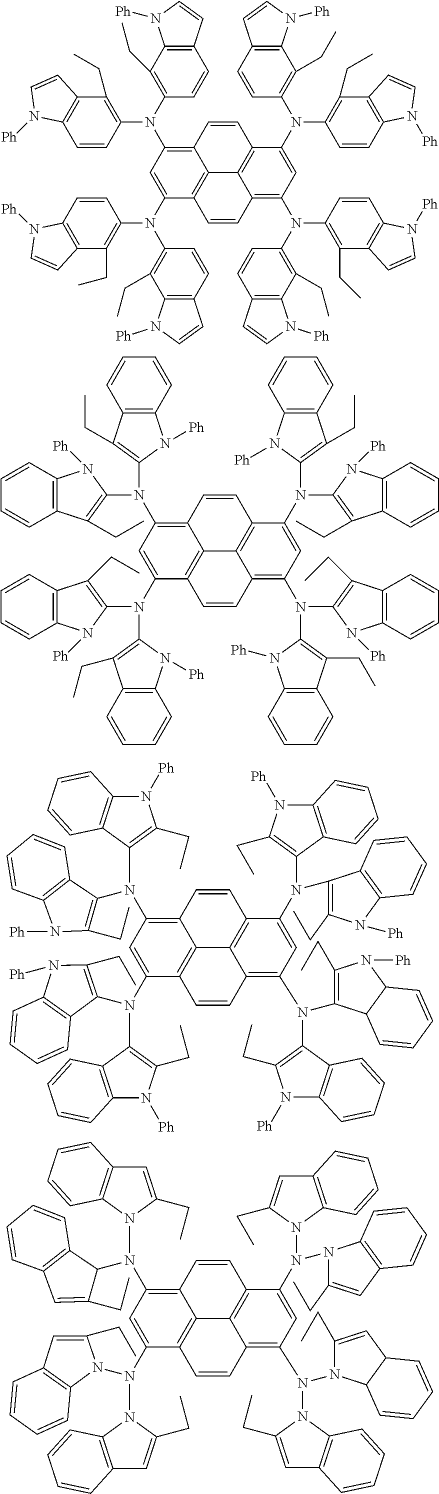

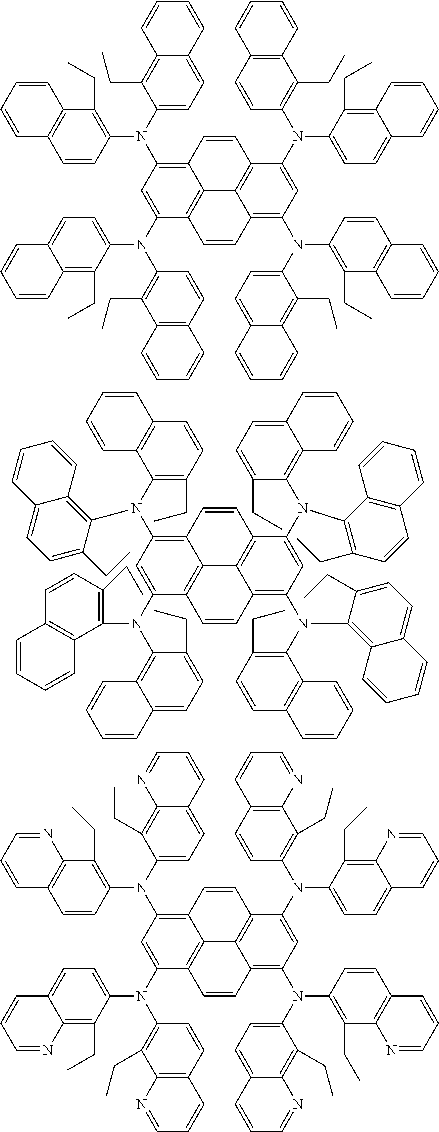

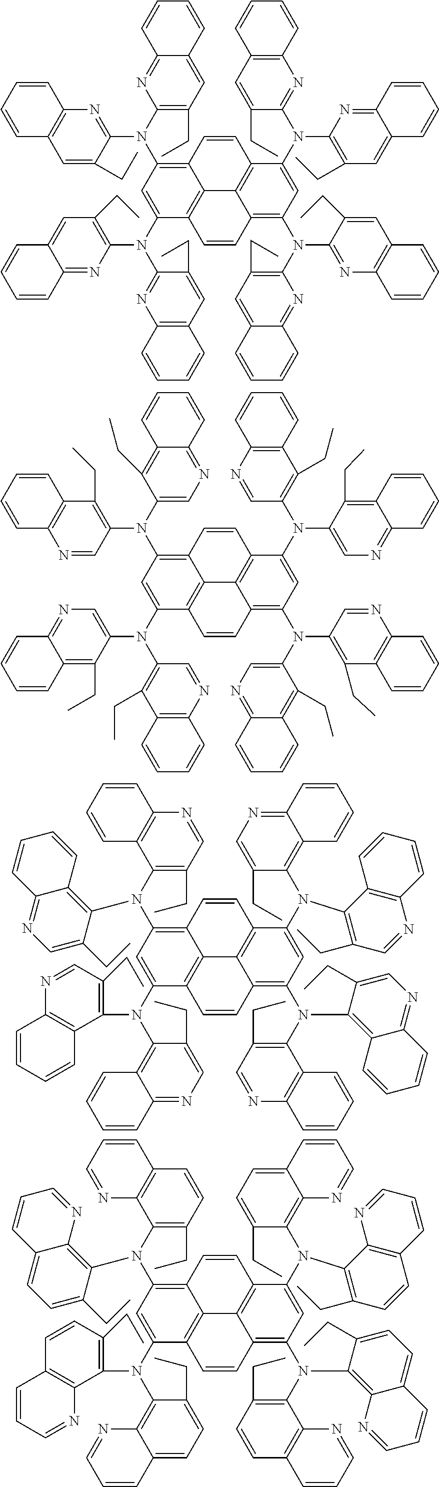

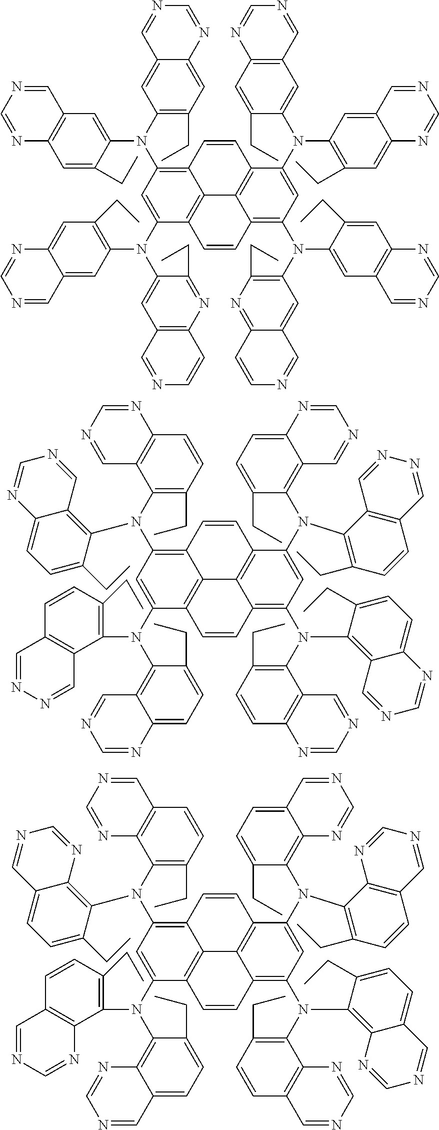

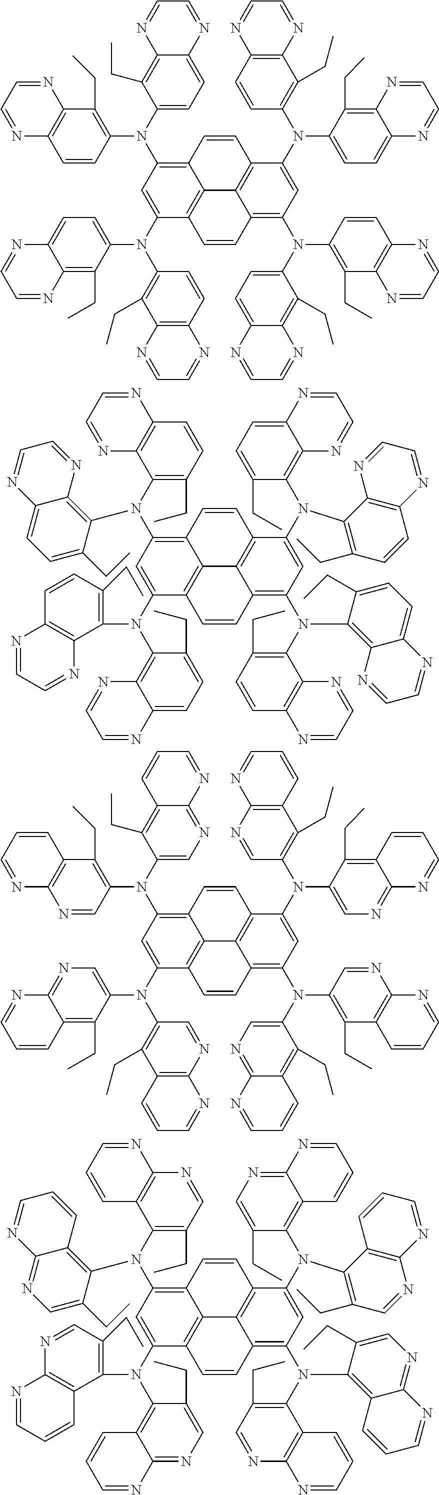

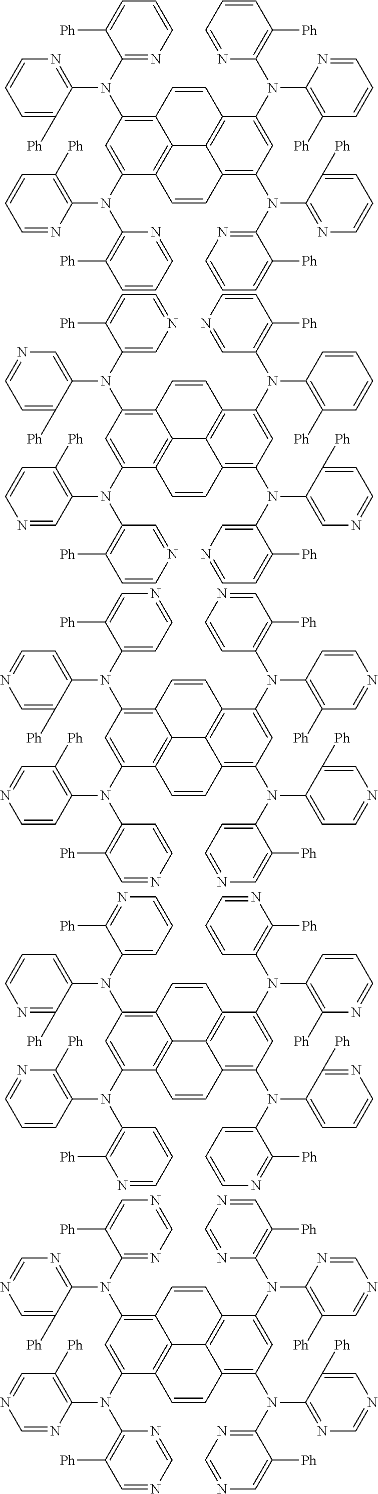

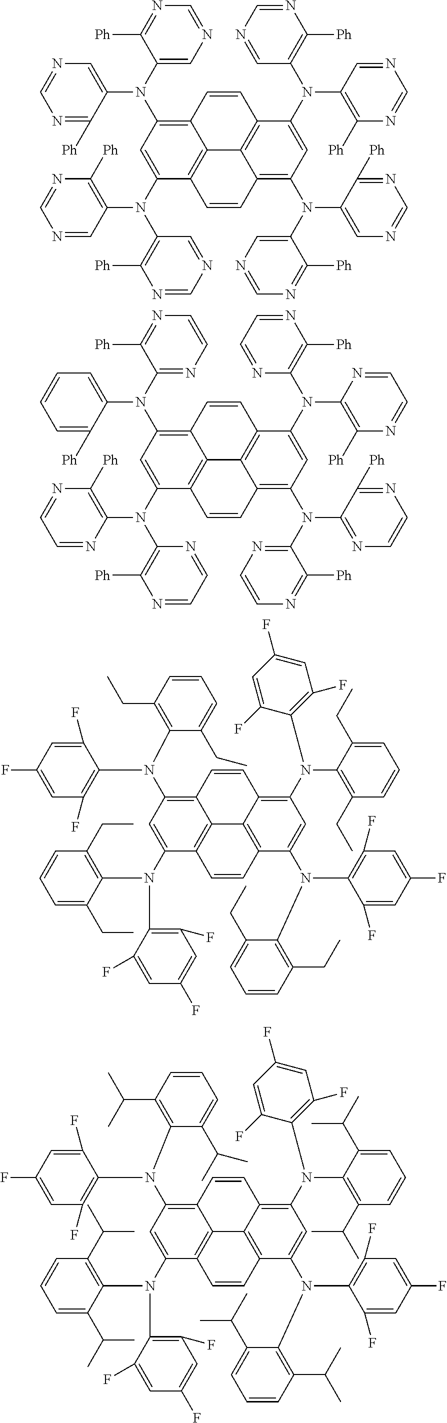

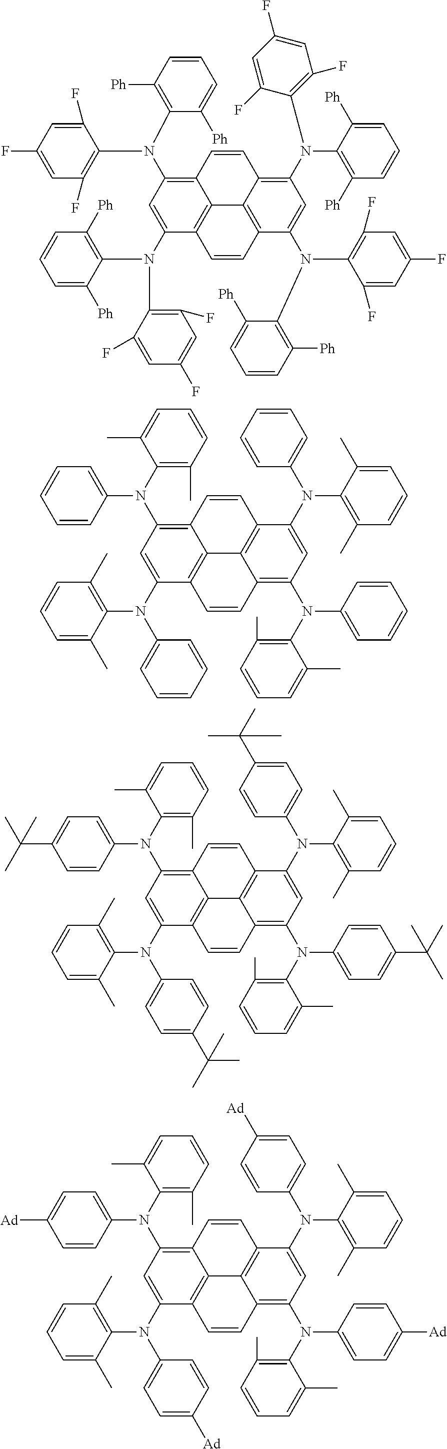

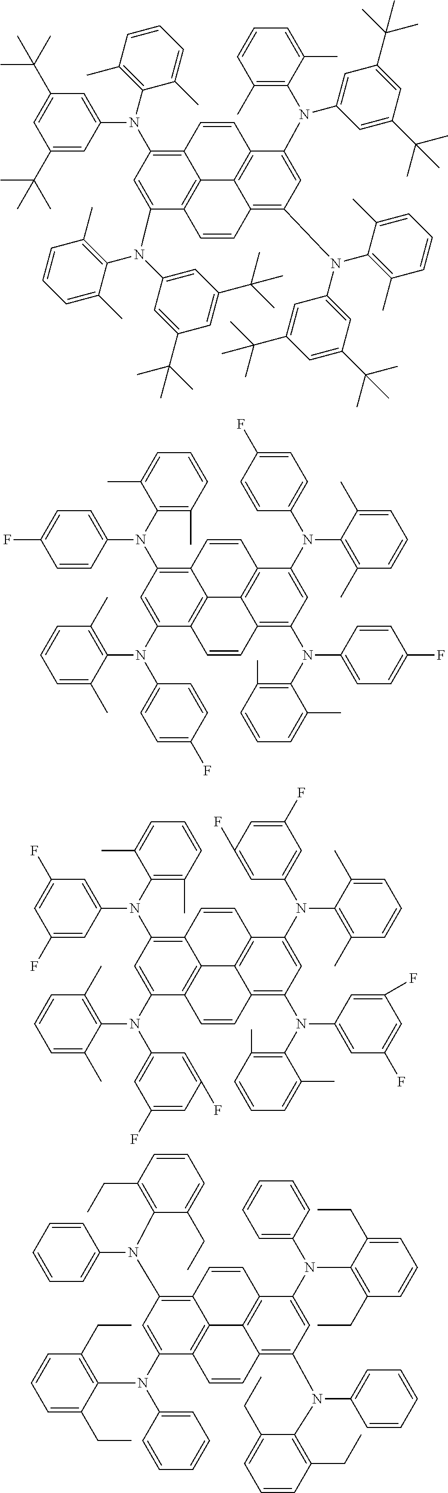

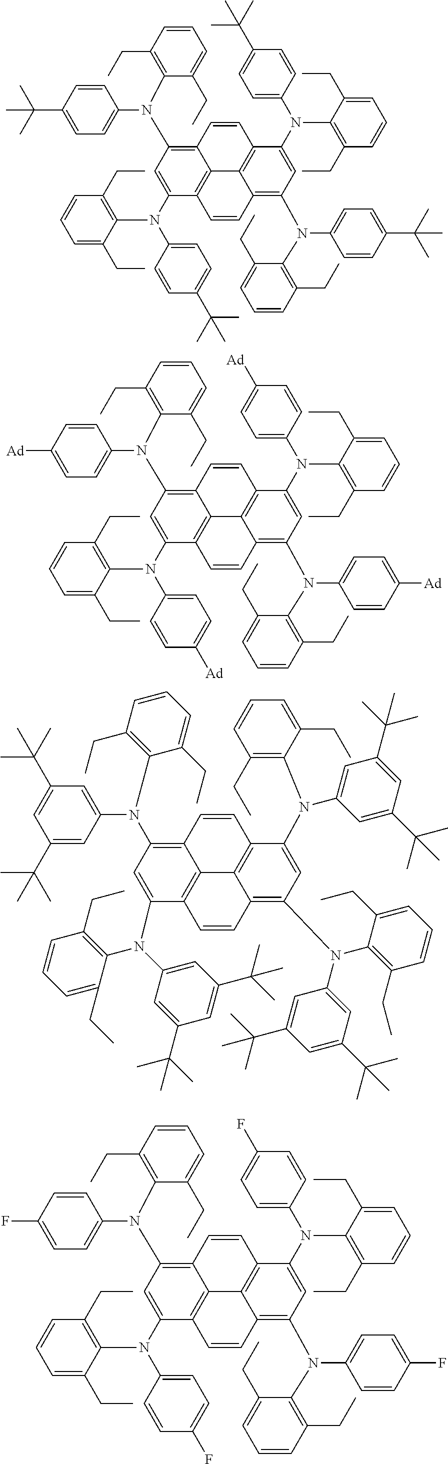

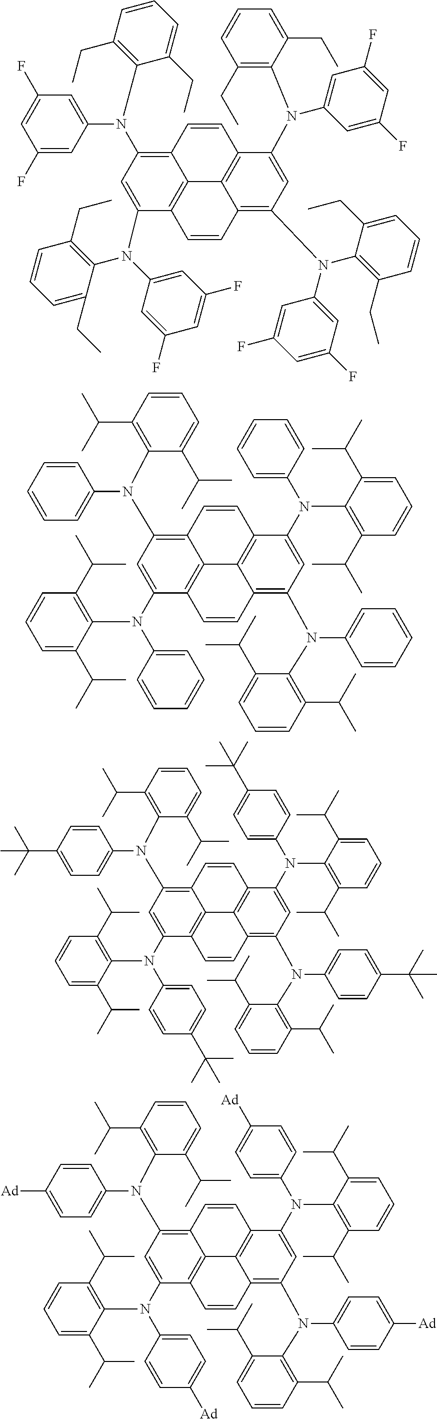

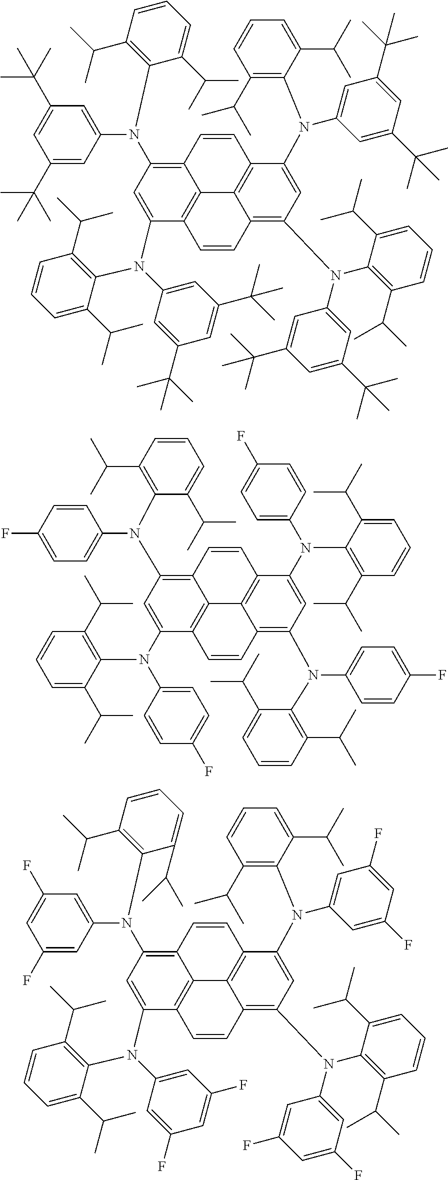

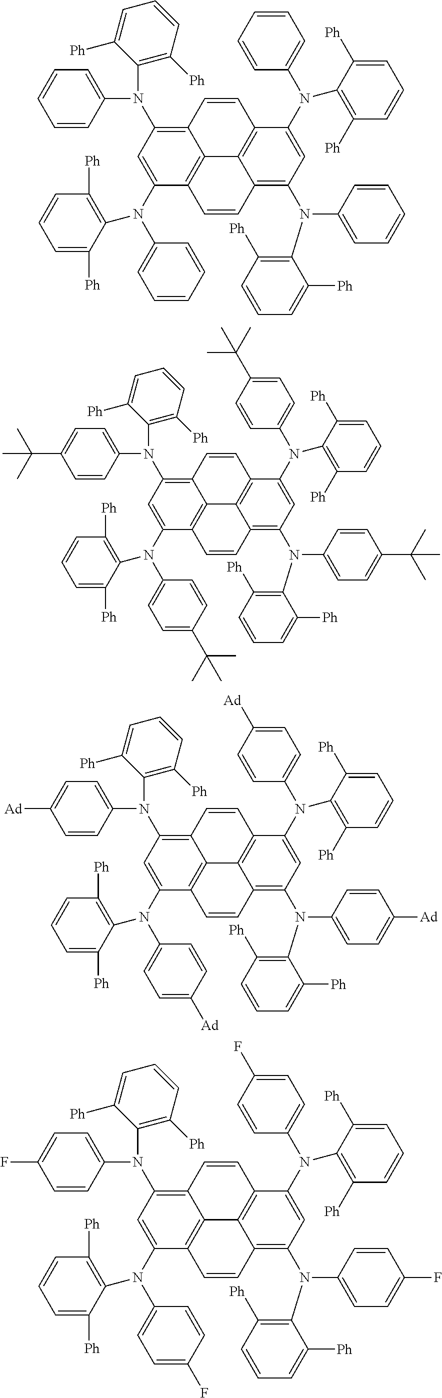

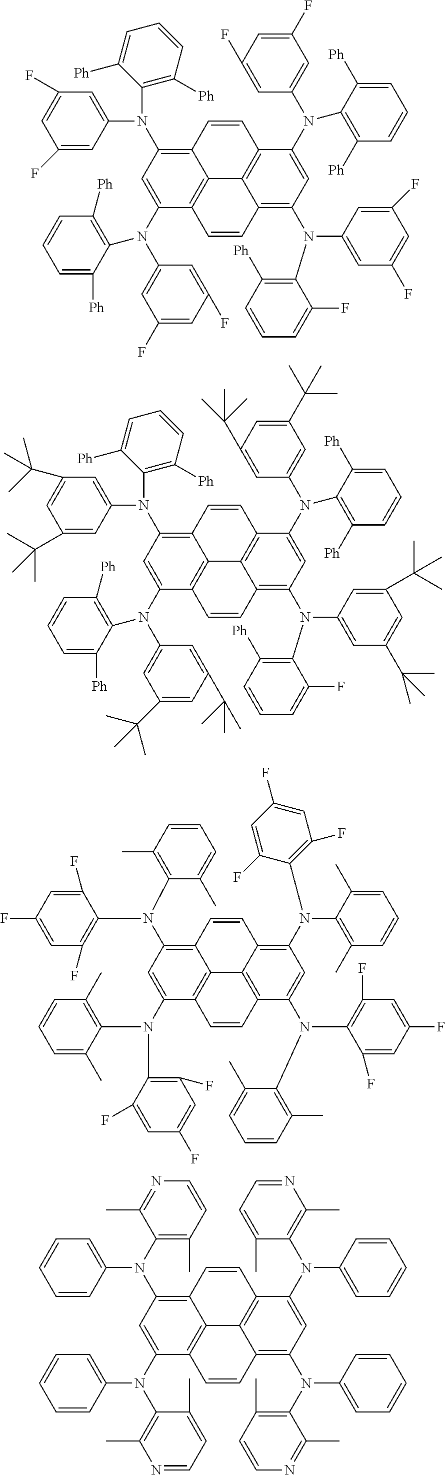

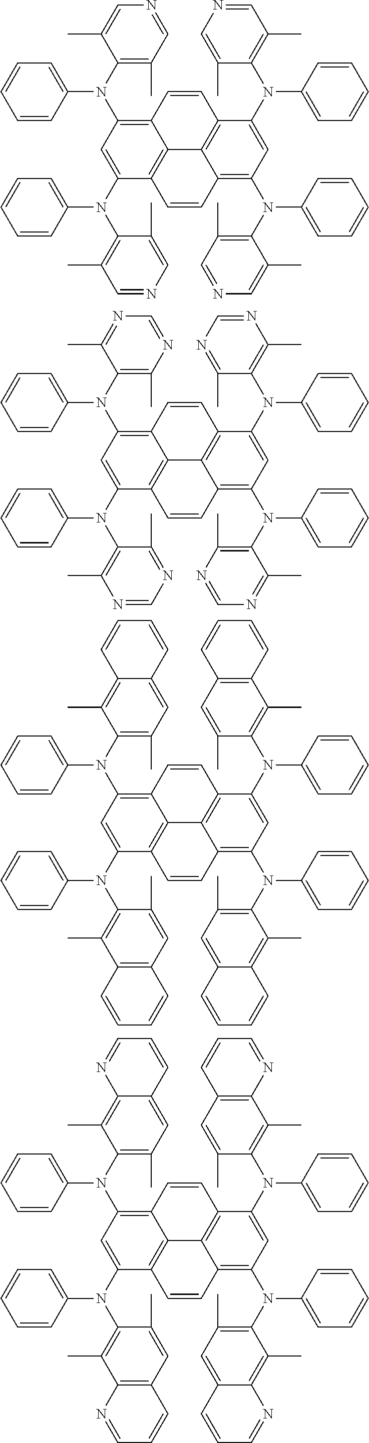

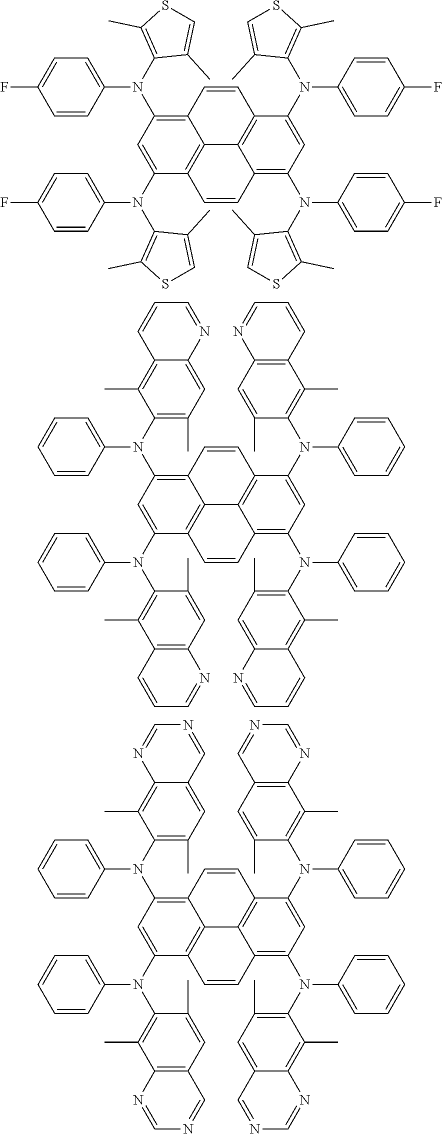

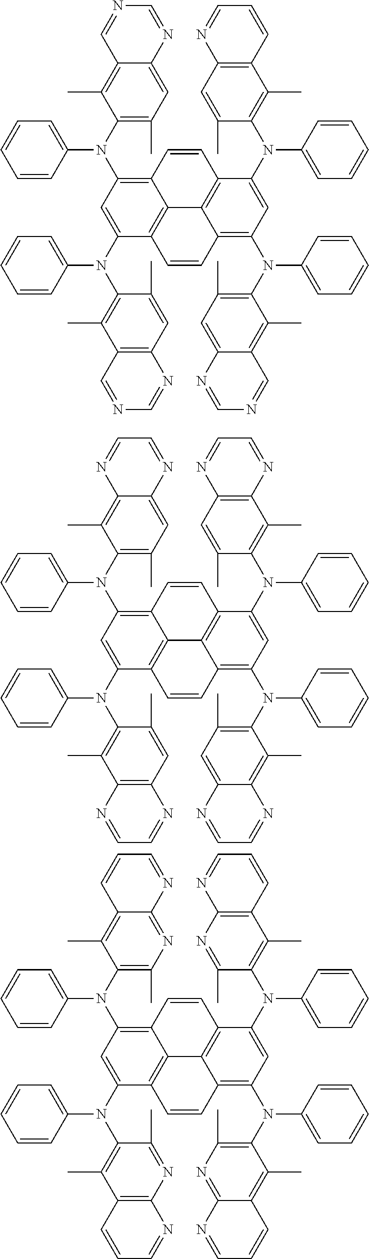

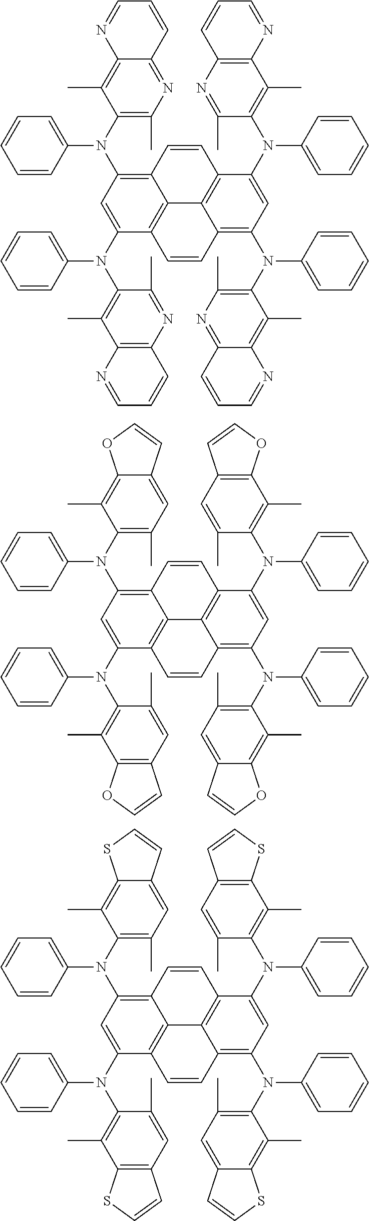

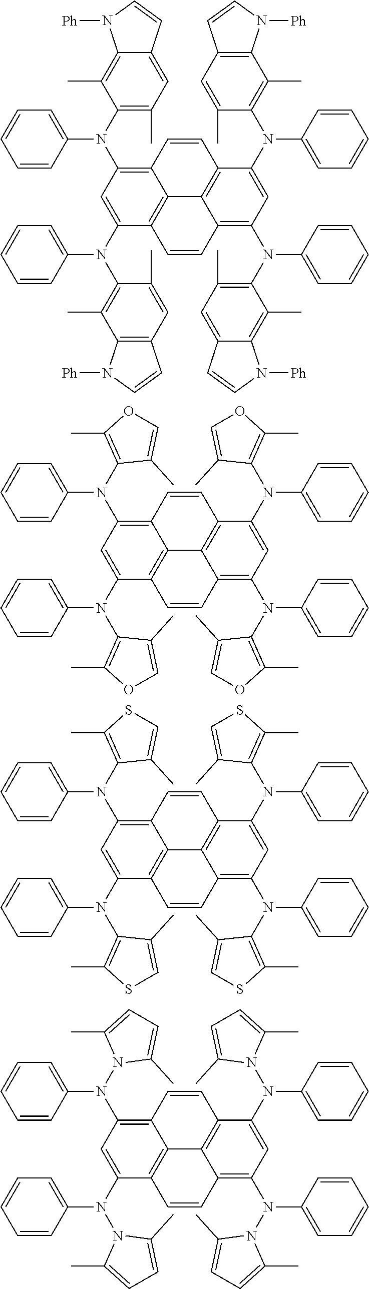

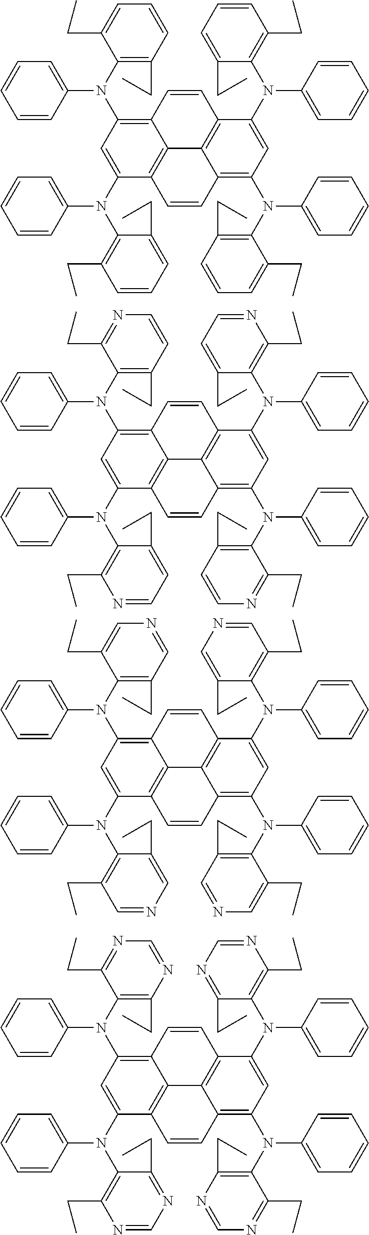

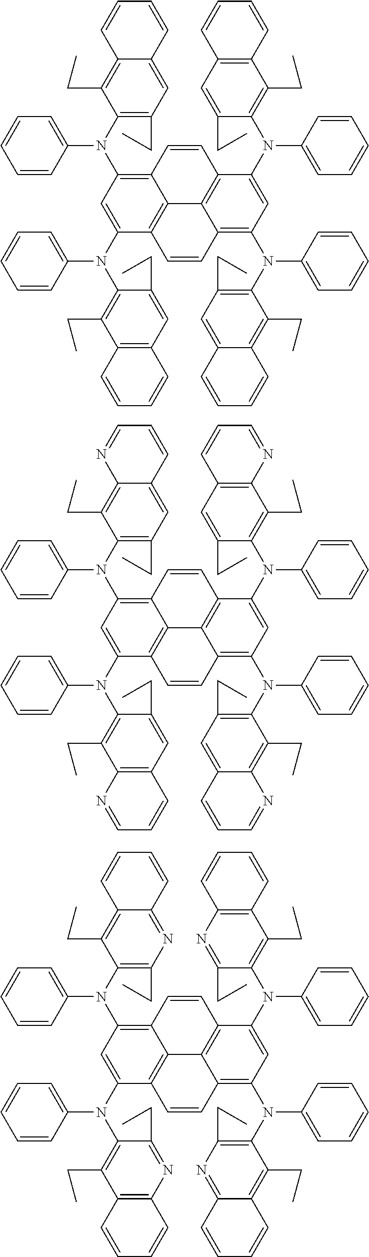

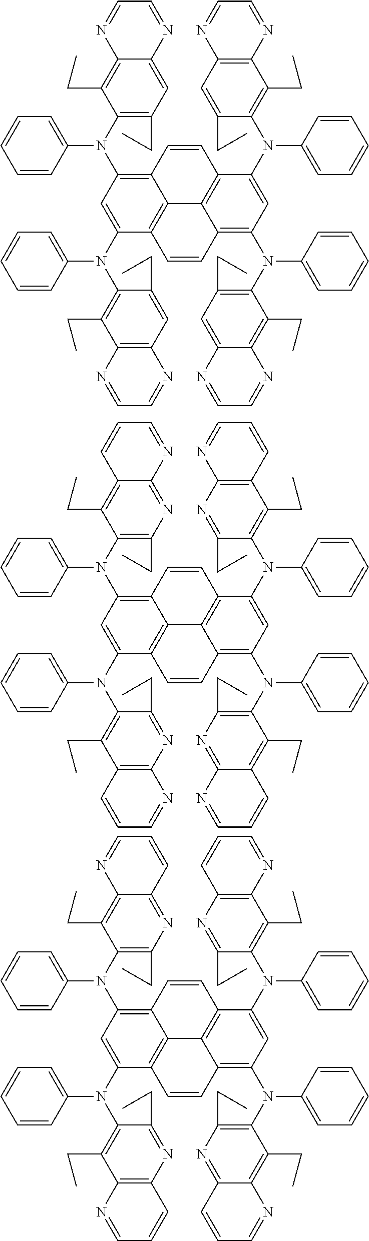

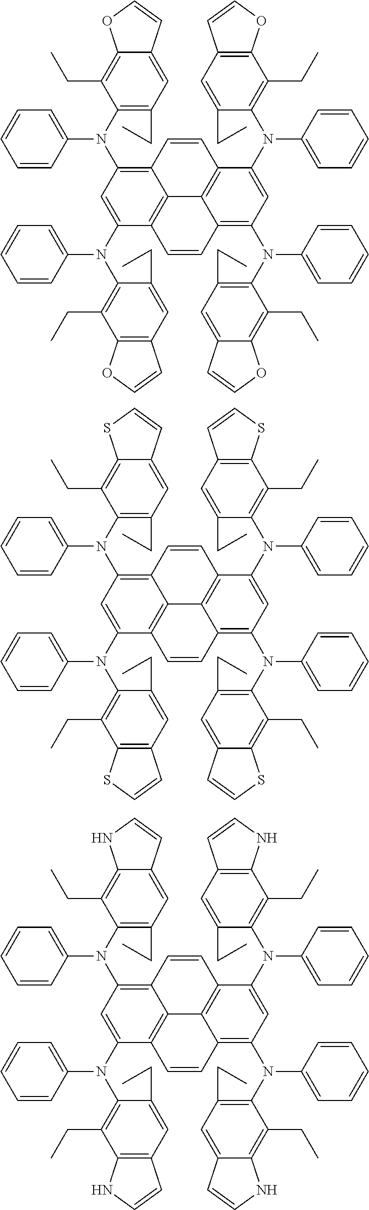

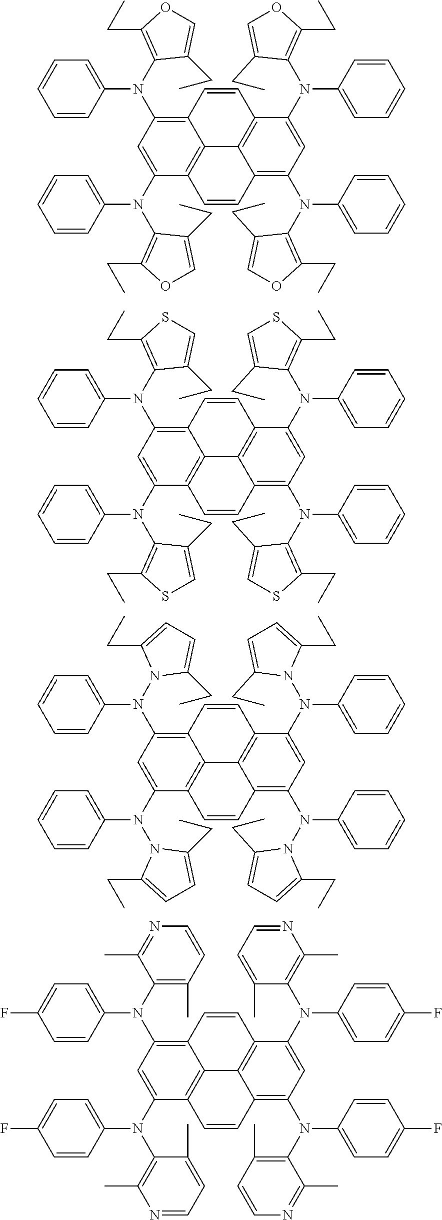

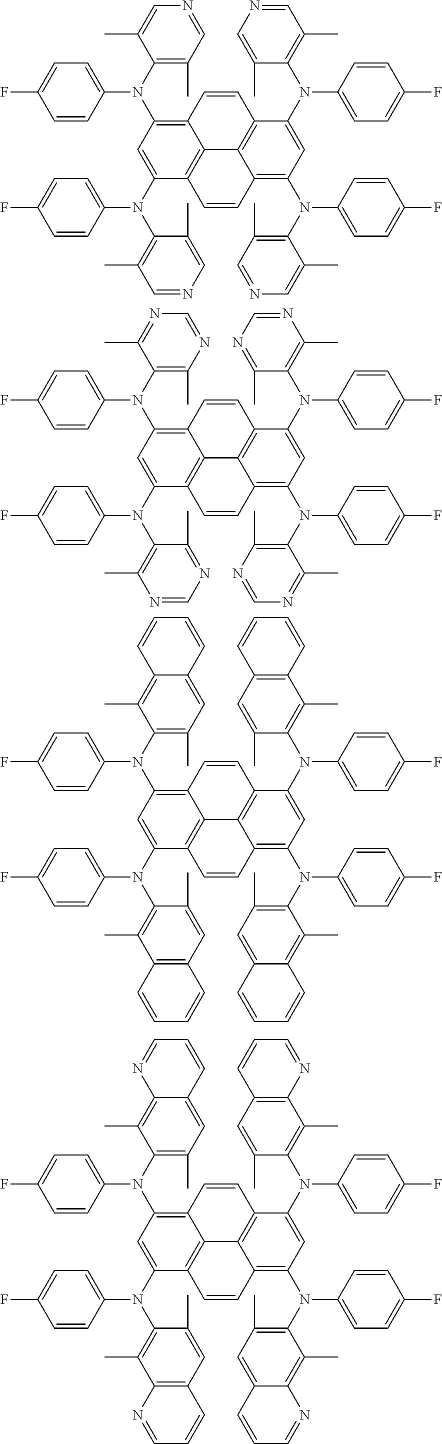

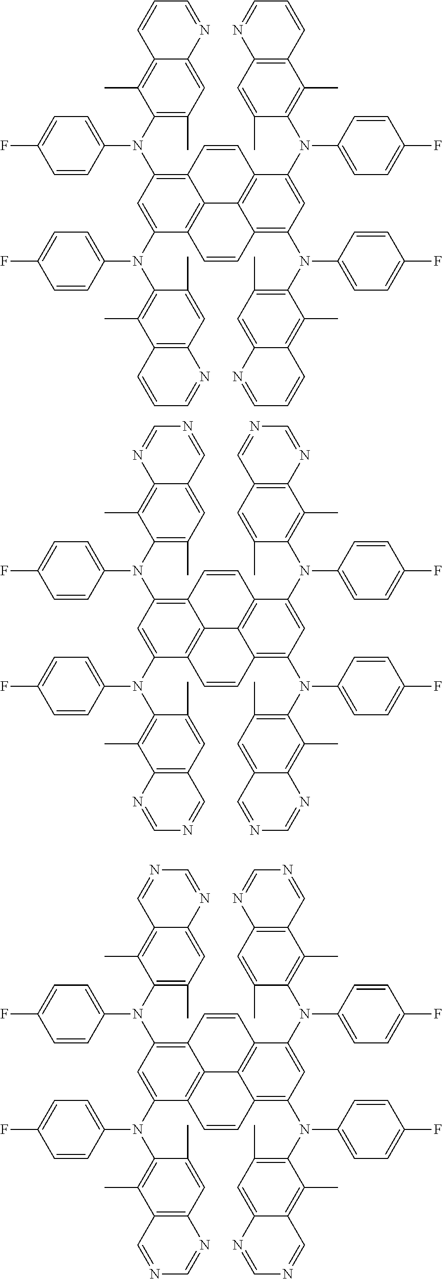

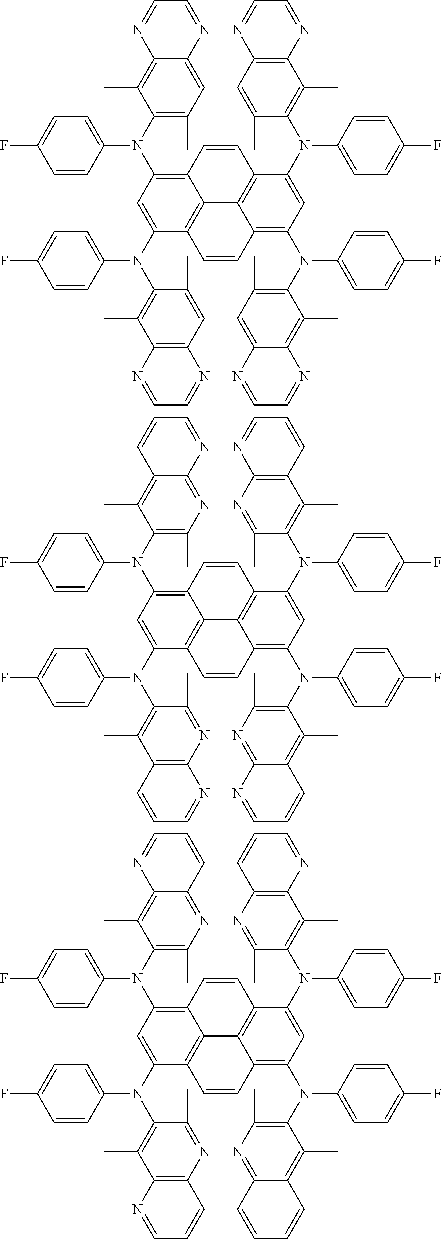

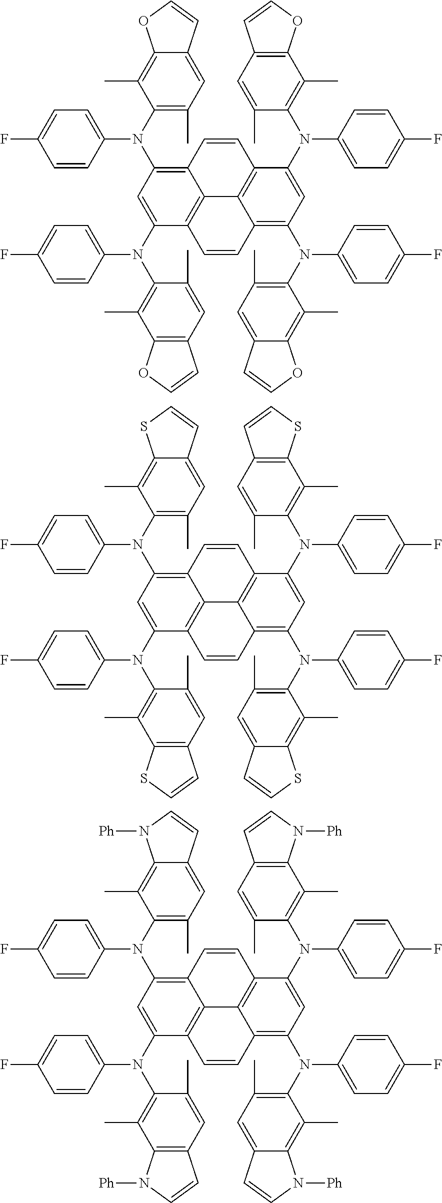

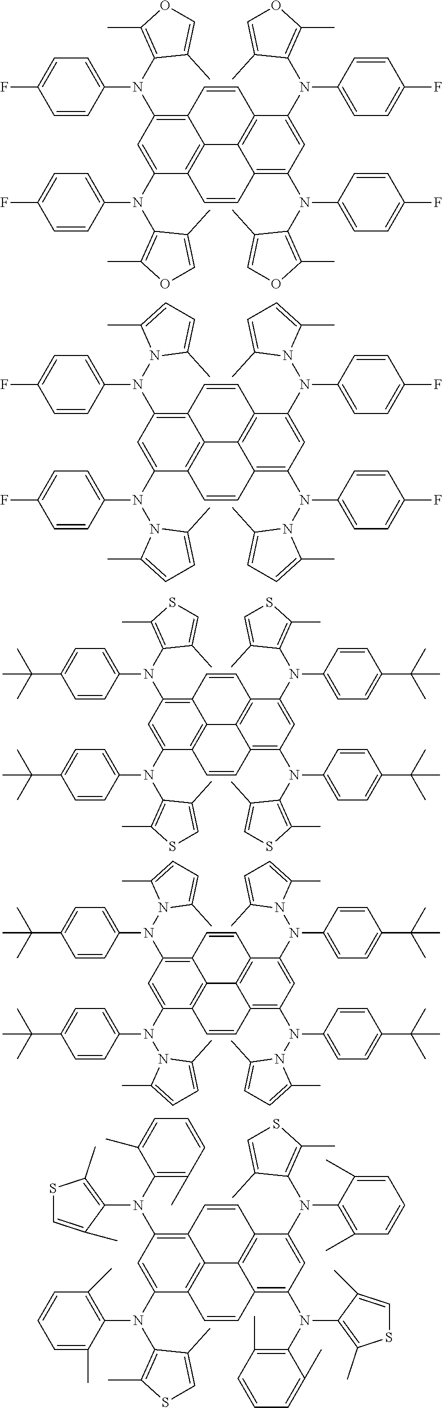

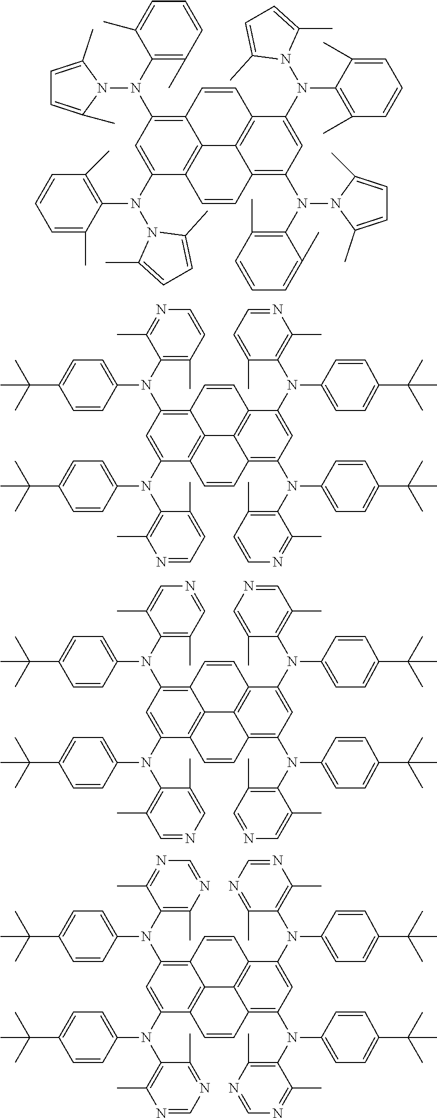

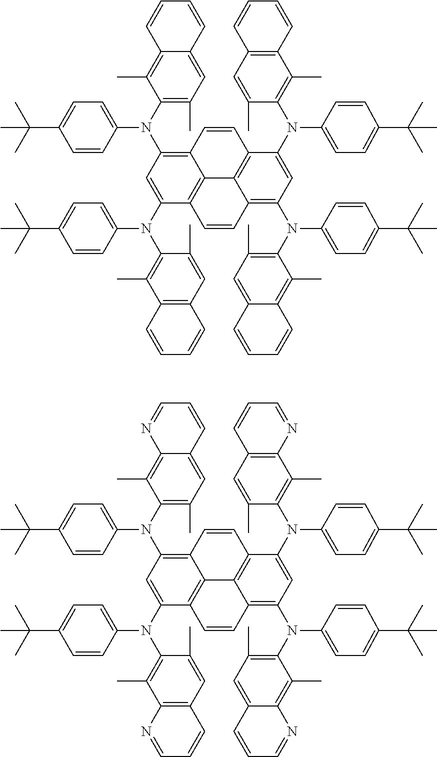

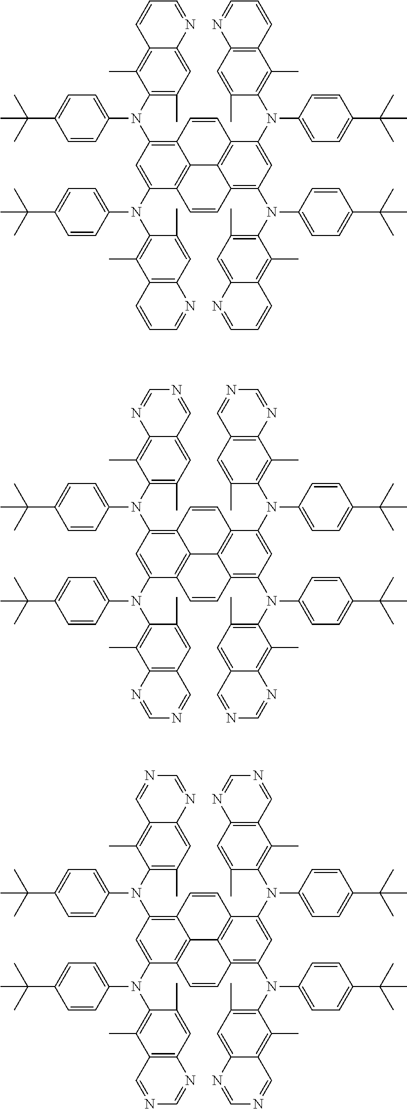

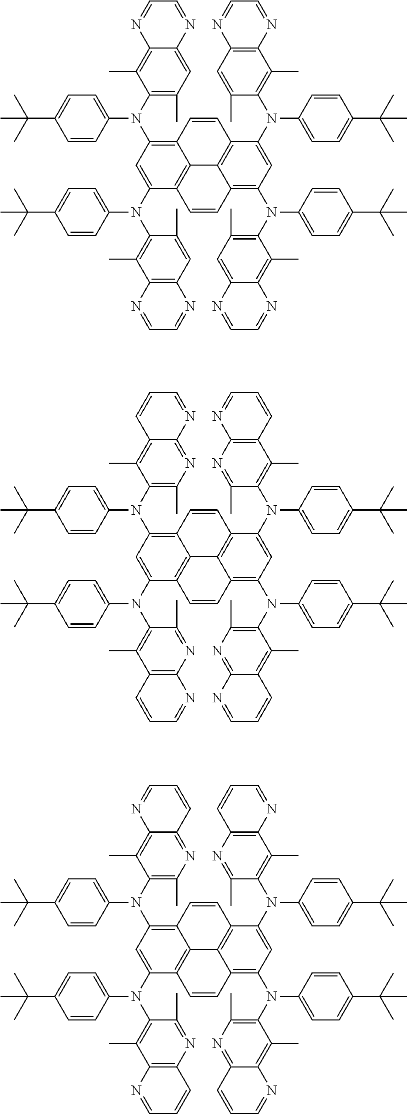

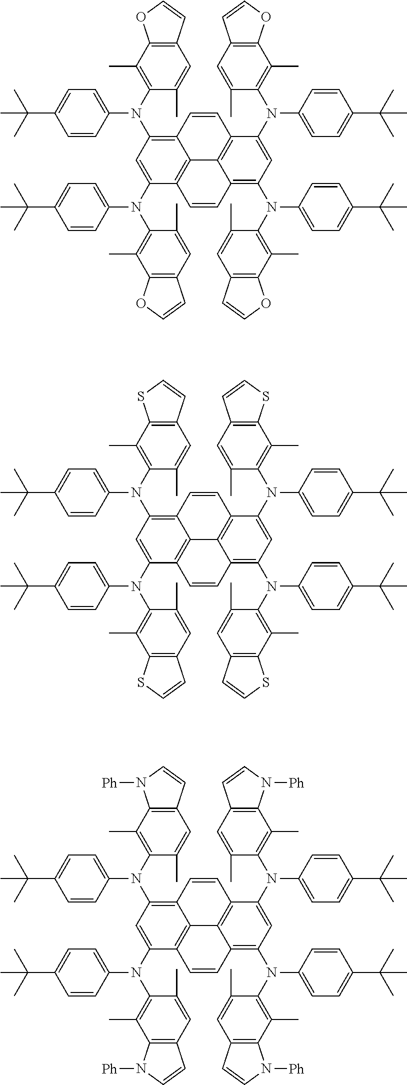

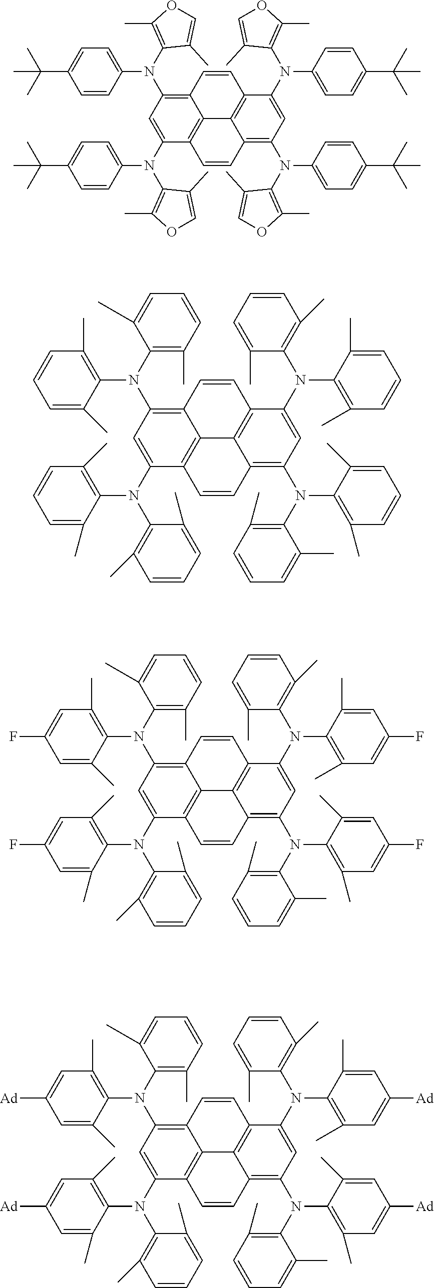

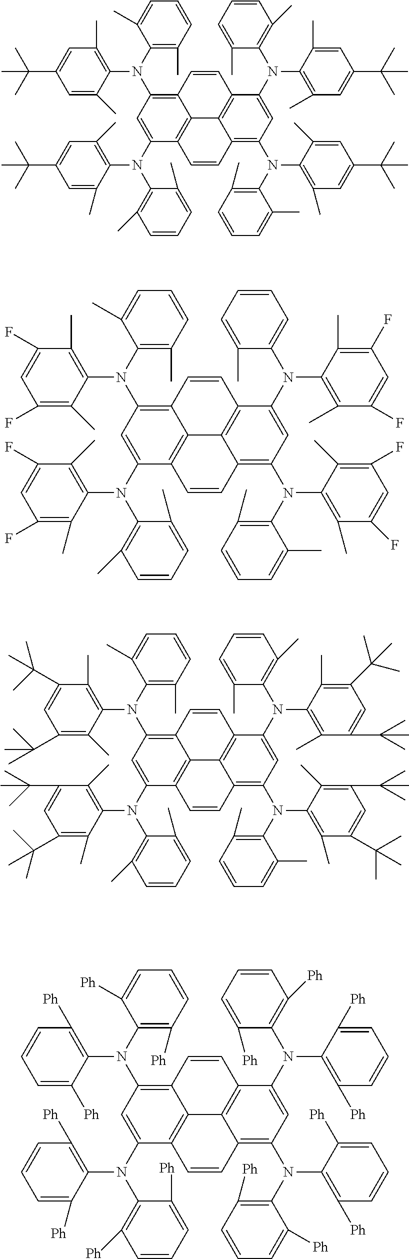

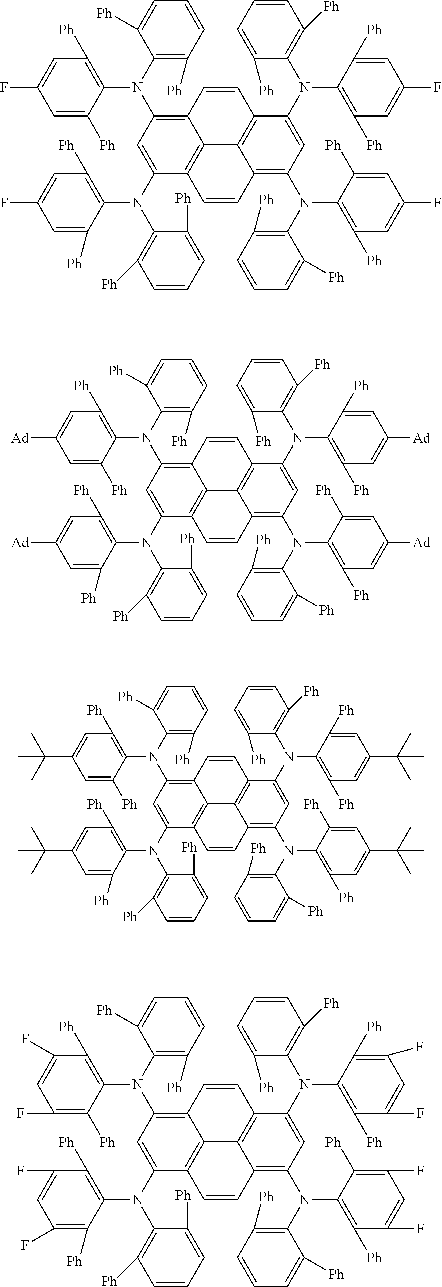

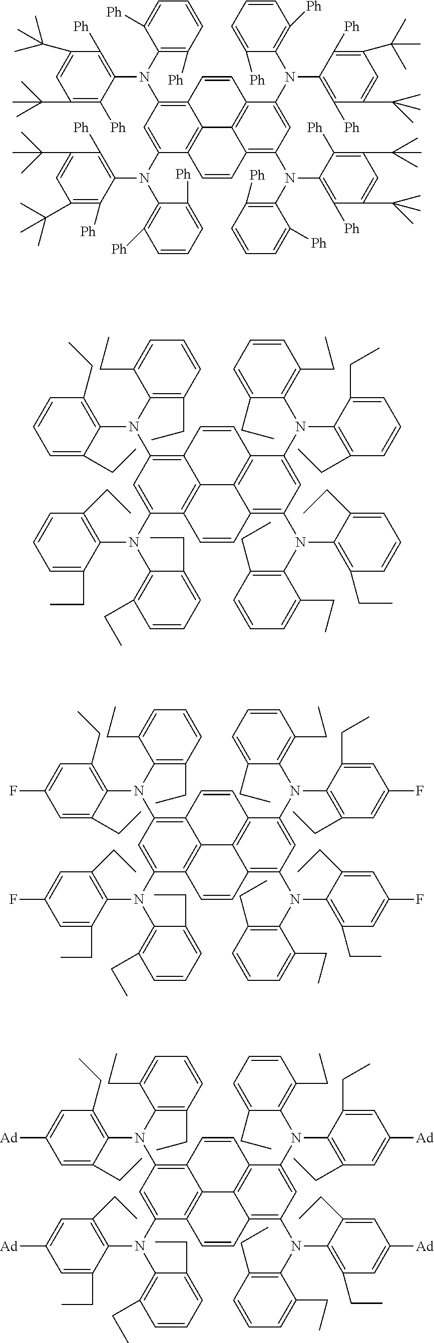

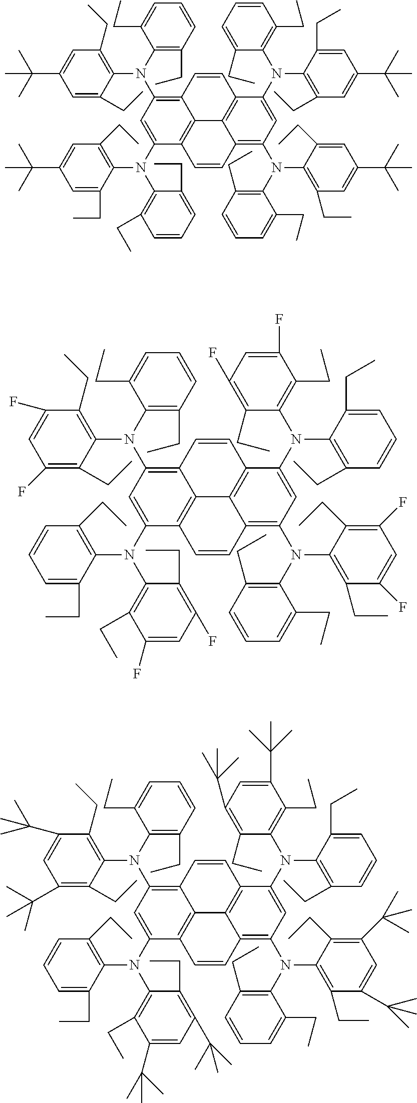

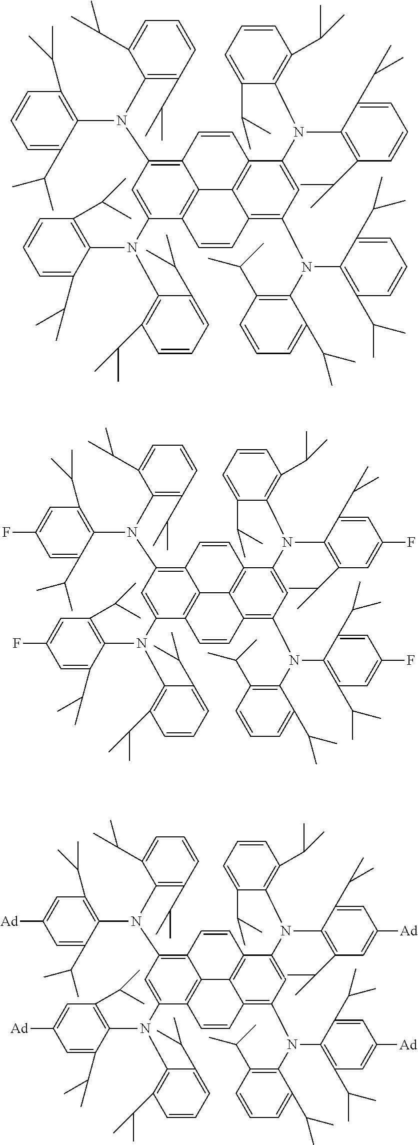

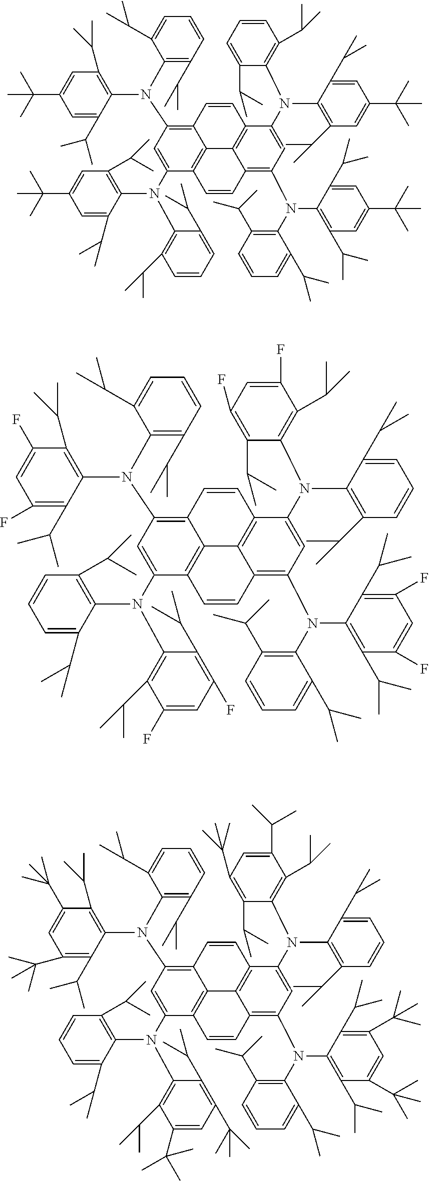

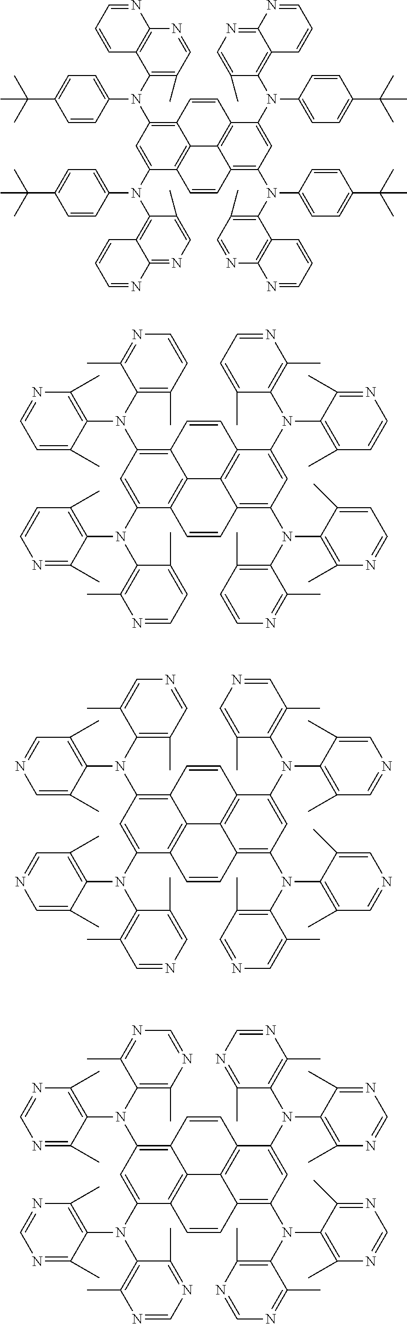

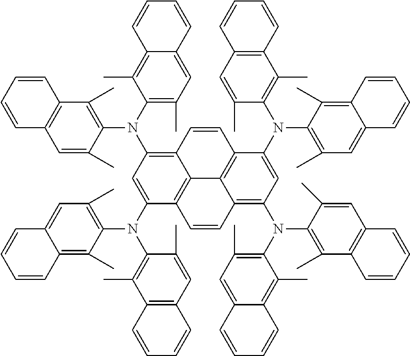 [SMILES: Cc1cc2ccccc2c(C)c1N(c1c(C)cc2ccccc2c1C)c1cc(N(c2c(C)cc3ccccc3c2C)c2c(C)cc3ccccc3c2C)c2ccc3c(N(c4c(C)cc5ccccc5c4C)c4c(C)cc5ccccc5c4C)cc(N(c4c(C)cc5ccccc5c4C)c4c(C)cc5ccccc5c4C)c4ccc1c2c43]

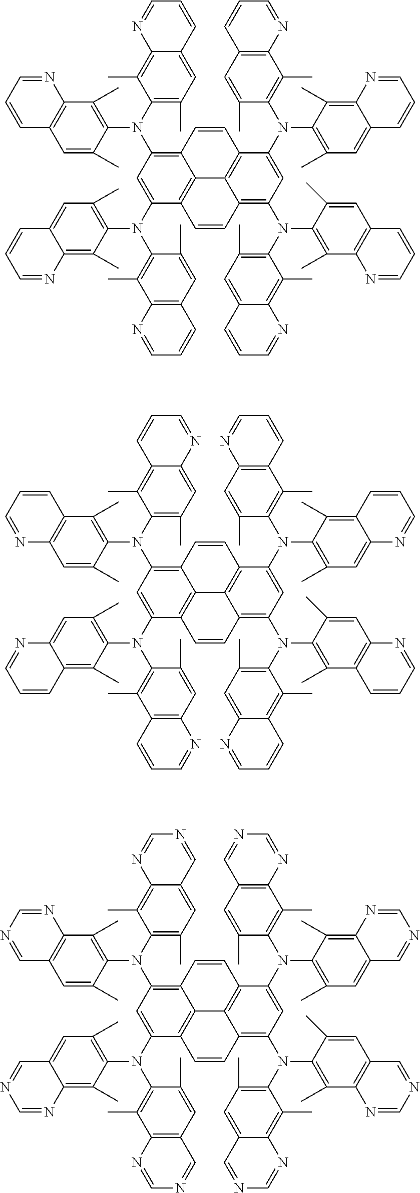

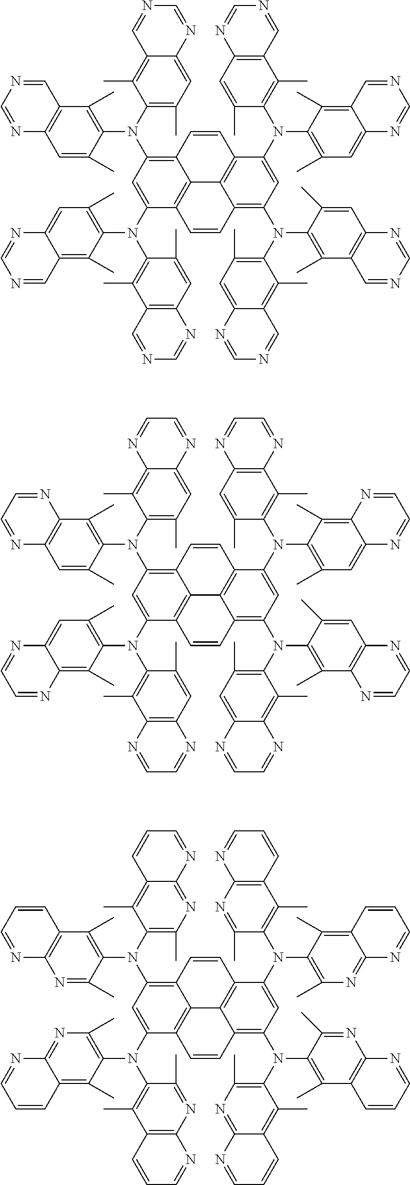

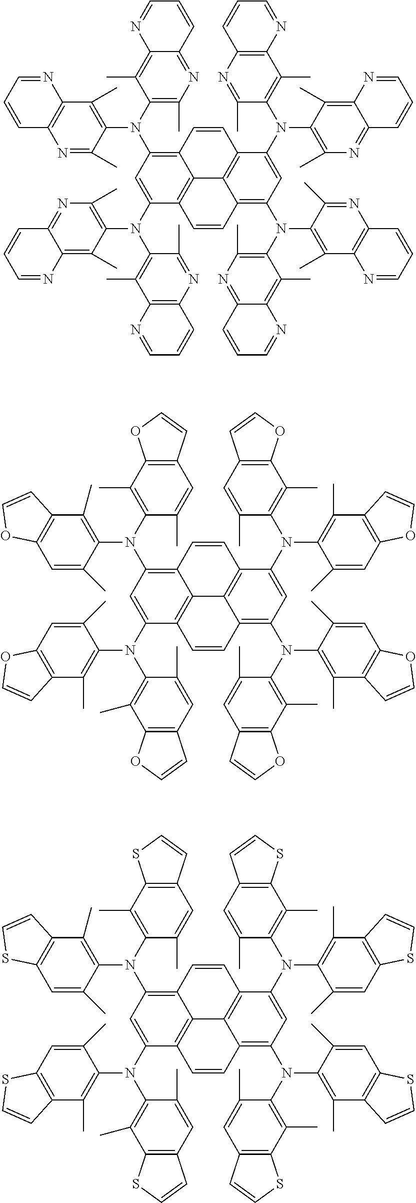

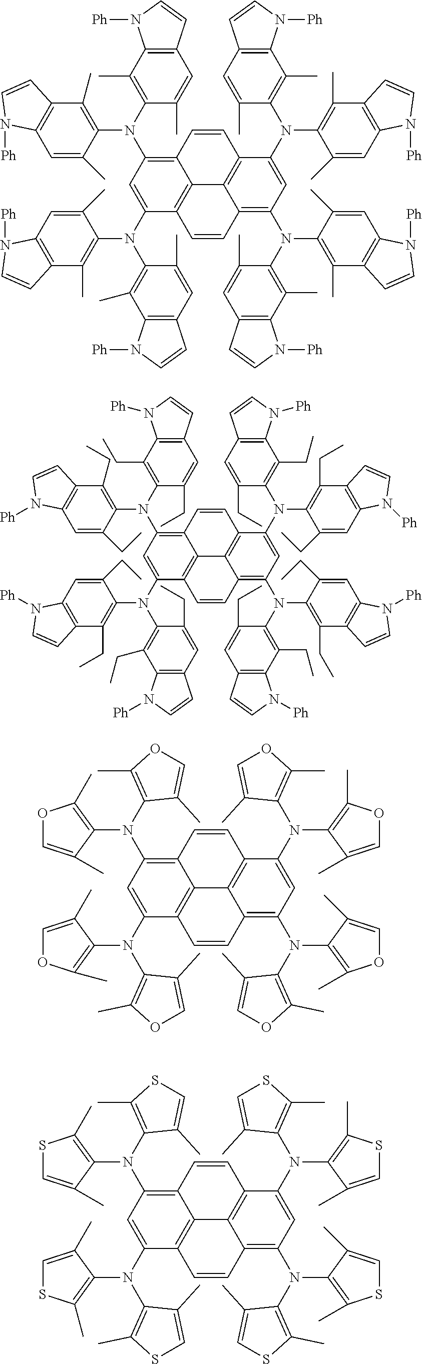

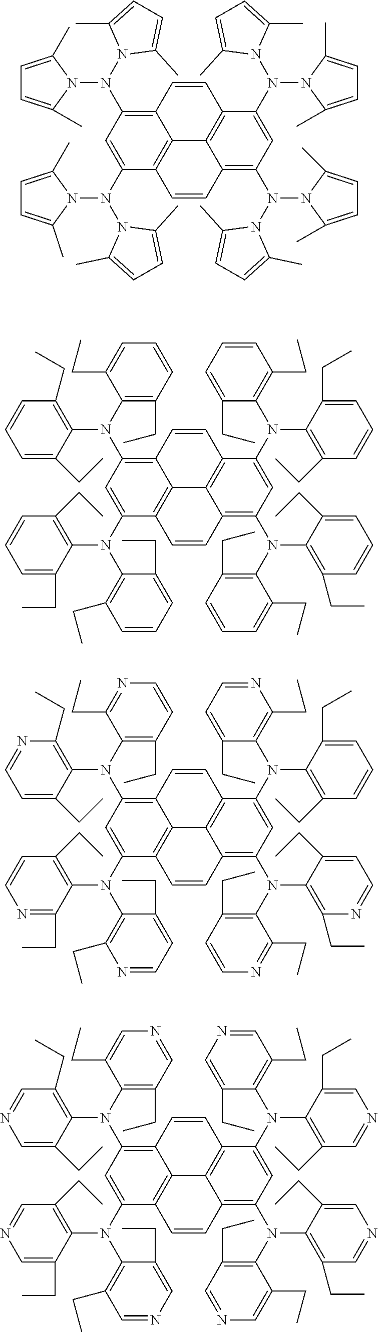

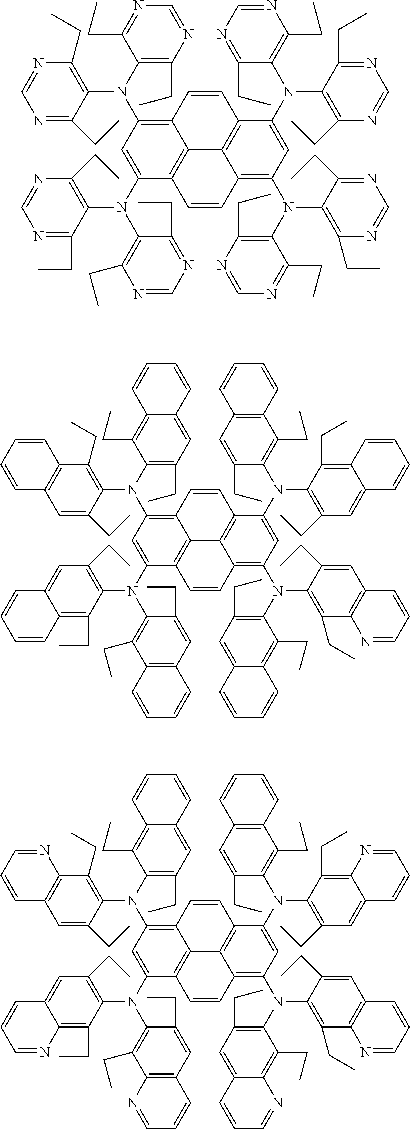

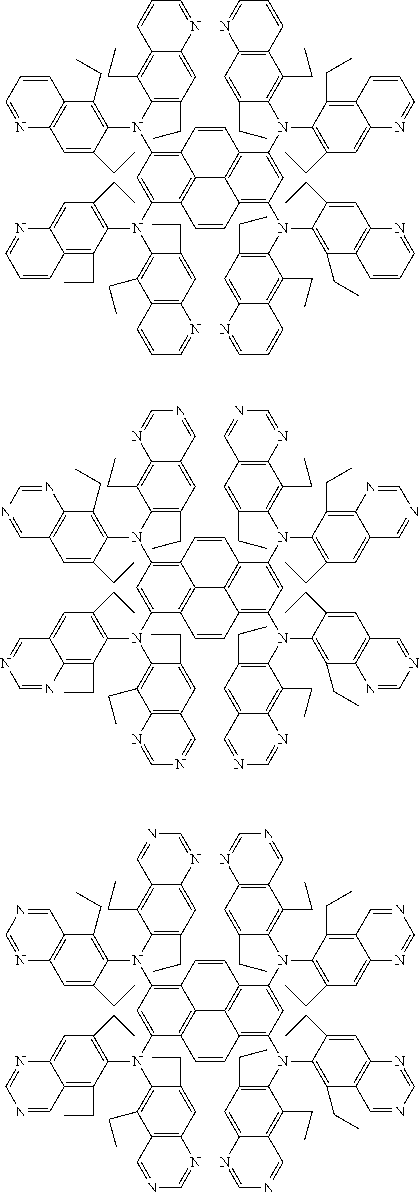

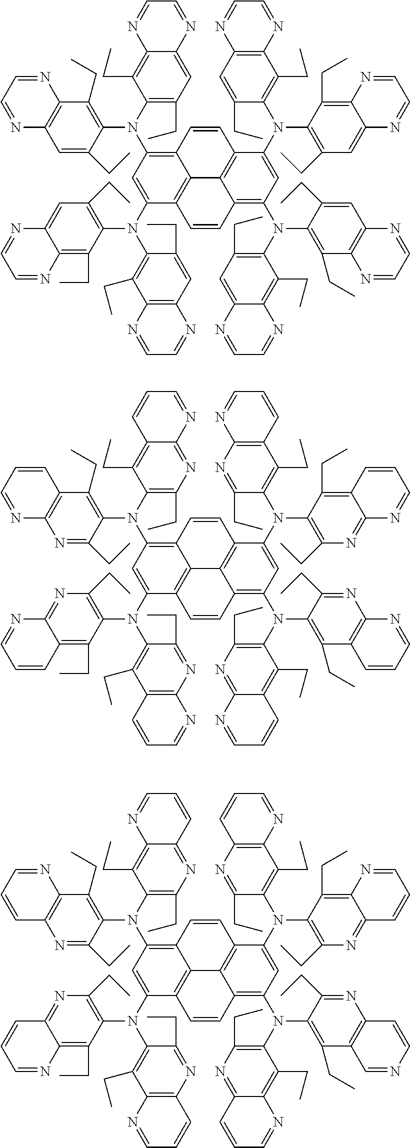

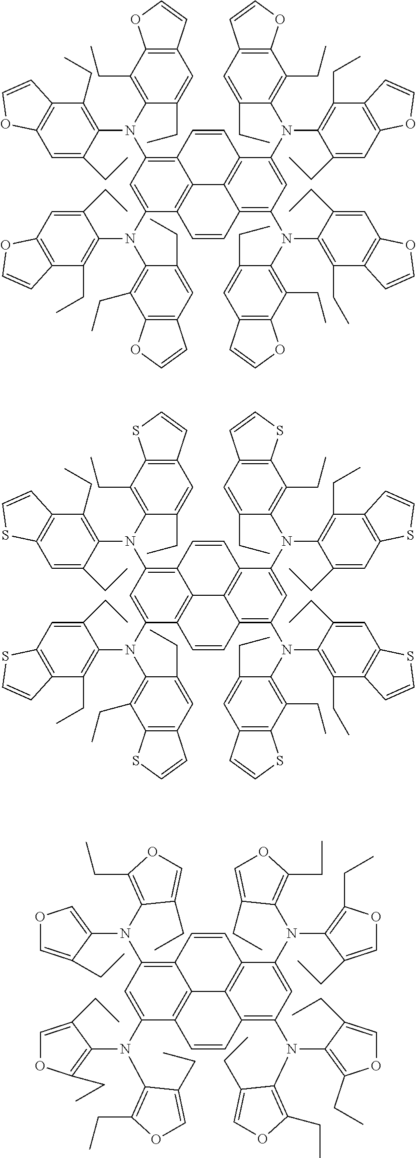

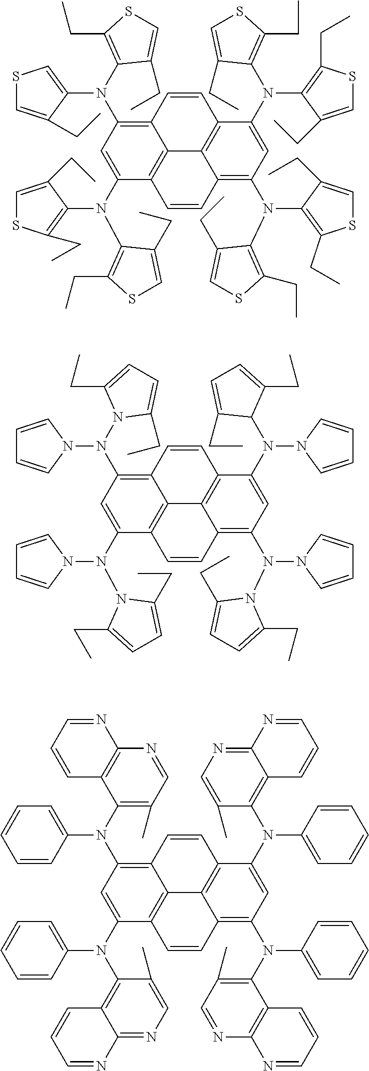

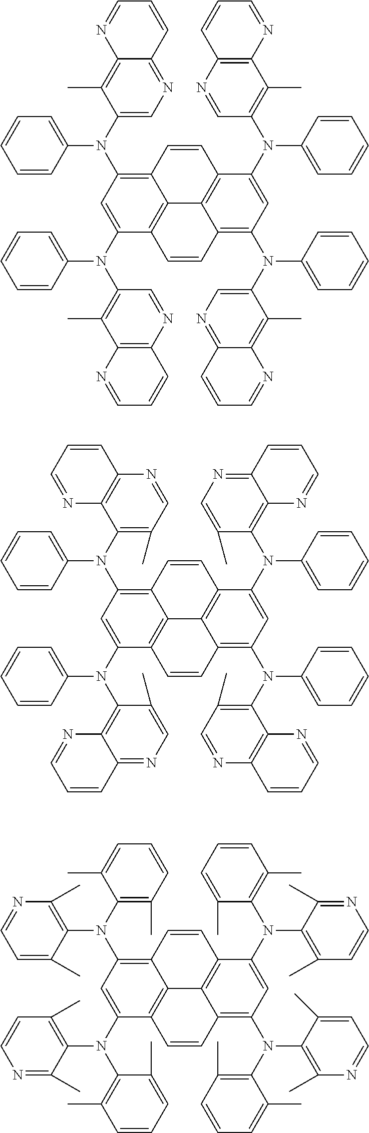

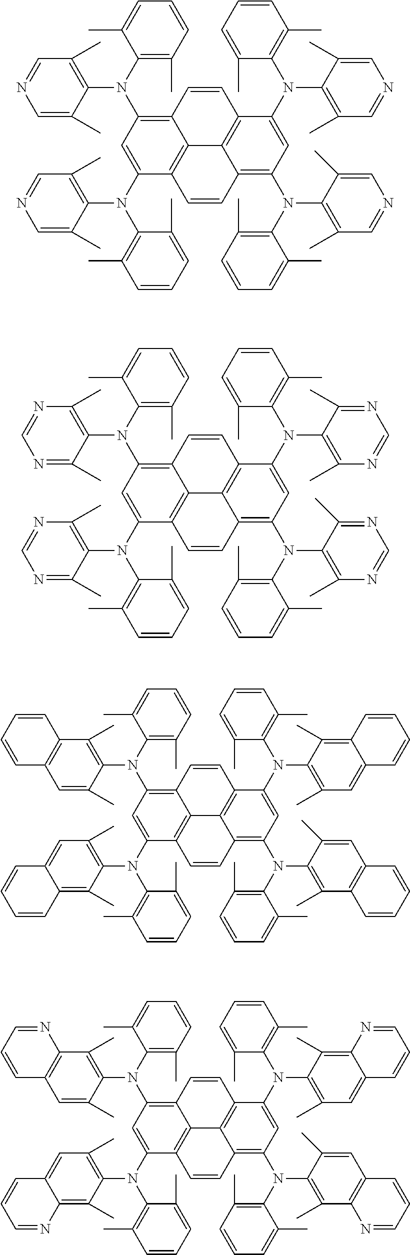

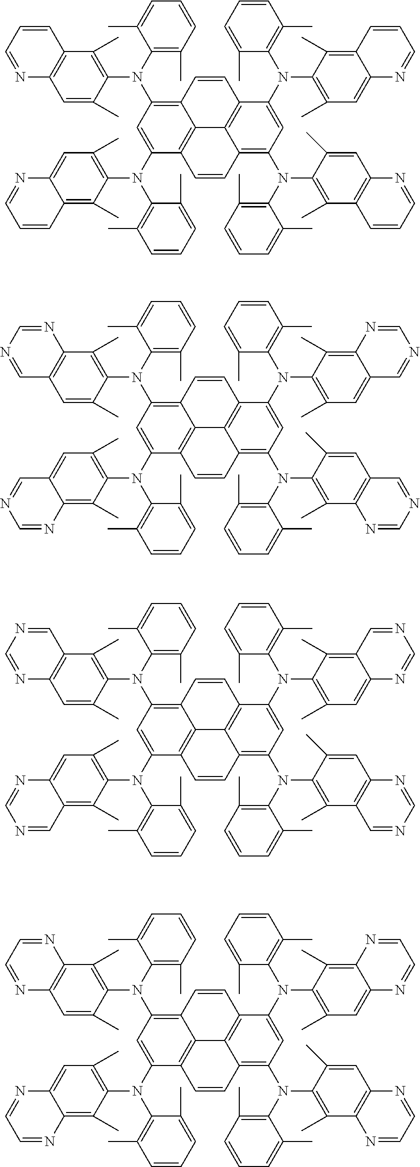

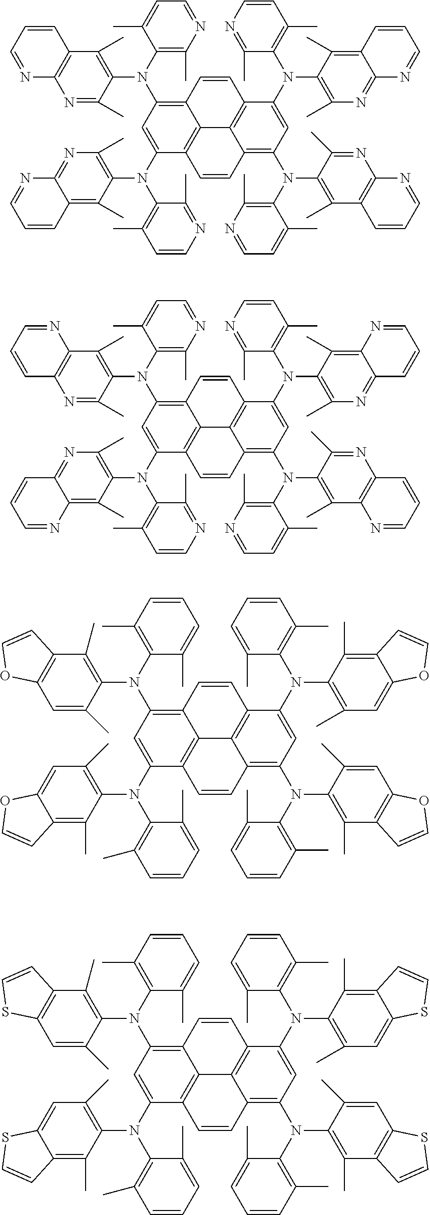

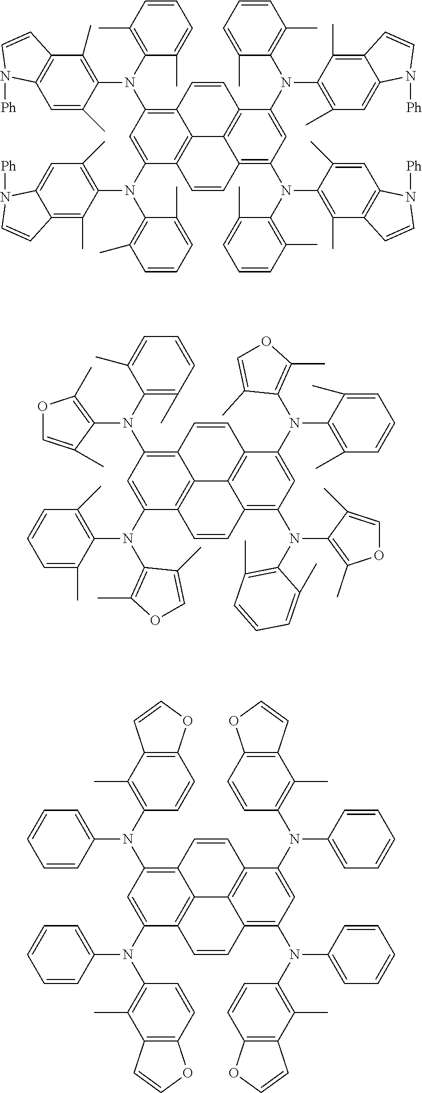 [SMILES: Cc1cccc(C)c1N(c1c(C)cc2c(ccn2-c2ccccc2)c1C)c1cc(N(c2c(C)cccc2C)c2c(C)cc3c(ccn3-c3ccccc3)c2C)c2ccc3c(N(c4c(C)cccc4C)c4c(C)cc5c(ccn5-c5ccccc5)c4C)cc(N(c4c(C)cccc4C)c4c(C)cc5c(ccn5-c5ccccc5)c4C)c4ccc1c2c43 ; Cc1cccc(C)c1N(c1c(C)coc1C)c1cc(N(c2c(C)cccc2C)c2c(C)coc2C)c2ccc3c(N(c4c(C)cccc4C)c4c(C)coc4C)cc(N(c4c(C)cccc4C)c4c(C)coc4C)c4ccc1c2c43 ; Cc1c(N(c2ccccc2)c2cc(N(c3ccccc3)c3ccc4occc4c3C)c3ccc4c(N(c5ccccc5)c5ccc6occc6c5C)cc(N(c5ccccc5)c5ccc6occc6c5C)c5ccc2c3c54)ccc2occc12]

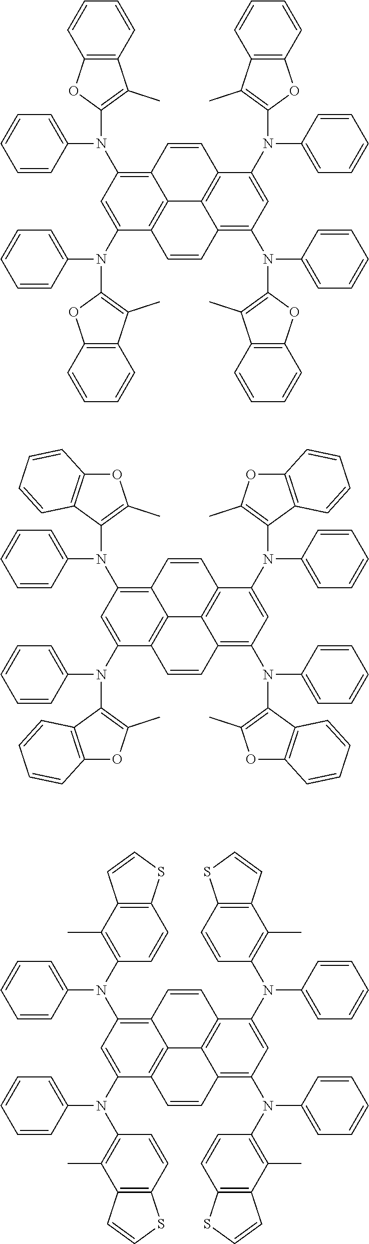 [SMILES: Cc1c(N(c2ccccc2)c2cc(N(c3ccccc3)c3oc4ccccc4c3C)c3ccc4c(N(c5ccccc5)c5oc6ccccc6c5C)cc(N(c5ccccc5)c5oc6ccccc6c5C)c5ccc2c3c54)oc2ccccc12 ; Cc1oc2ccccc2c1N(c1ccccc1)c1cc(N(c2ccccc2)c2c(C)oc3ccccc23)c2ccc3c(N(c4ccccc4)c4c(C)oc5ccccc45)cc(N(c4ccccc4)c4c(C)oc5ccccc45)c4ccc1c2c43 ; Cc1c(N(c2ccccc2)c2cc(N(c3ccccc3)c3ccc4sccc4c3C)c3ccc4c(N(c5ccccc5)c5ccc6sccc6c5C)cc(N(c5ccccc5)c5ccc6sccc6c5C)c5ccc2c3c54)ccc2sccc12]

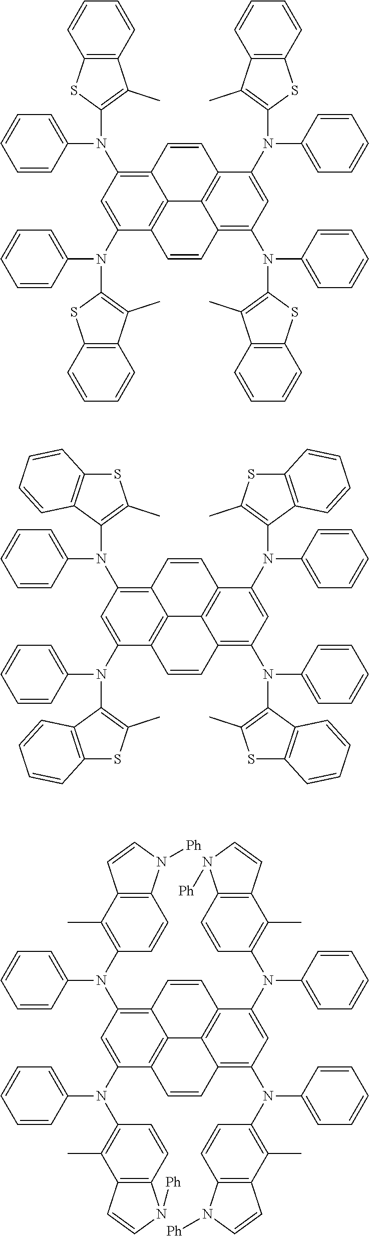 [SMILES: Cc1c(N(c2ccccc2)c2cc(N(c3ccccc3)c3sc4ccccc4c3C)c3ccc4c(N(c5ccccc5)c5sc6ccccc6c5C)cc(N(c5ccccc5)c5sc6ccccc6c5C)c5ccc2c3c54)sc2ccccc12 ; Cc1sc2ccccc2c1N(c1ccccc1)c1cc(N(c2ccccc2)c2c(C)sc3ccccc23)c2ccc3c(N(c4ccccc4)c4c(C)sc5ccccc45)cc(N(c4ccccc4)c4c(C)sc5ccccc45)c4ccc1c2c43 ; Cc1c(N(c2ccccc2)c2cc(N(c3ccccc3)c3ccc4c(ccn4-c4ccccc4)c3C)c3ccc4c(N(c5ccccc5)c5ccc6c(ccn6-c6ccccc6)c5C)cc(N(c5ccccc5)c5ccc6c(ccn6-c6ccccc6)c5C)c5ccc2c3c54)ccc2c1ccn2-c1ccccc1]

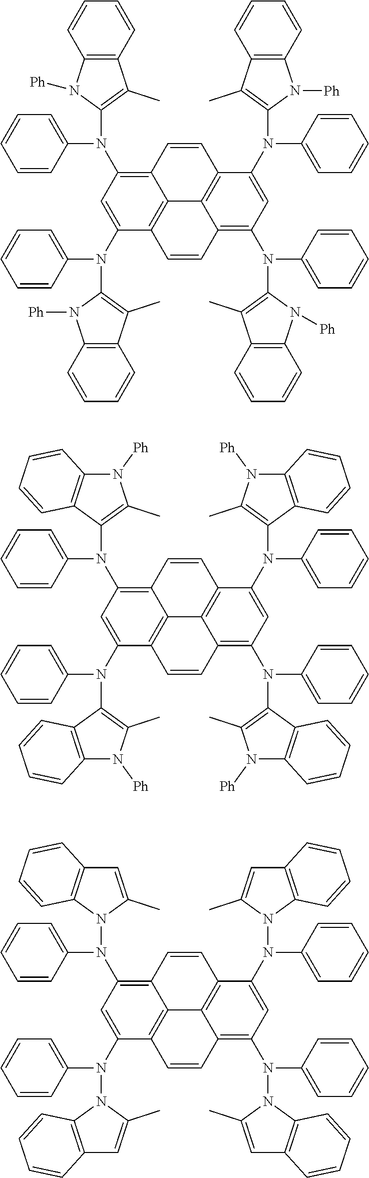 [SMILES: Cc1c(N(c2ccccc2)c2cc(N(c3ccccc3)c3c(C)c4ccccc4n3-c3ccccc3)c3ccc4c(N(c5ccccc5)c5c(C)c6ccccc6n5-c5ccccc5)cc(N(c5ccccc5)c5c(C)c6ccccc6n5-c5ccccc5)c5ccc2c3c54)n(-c2ccccc2)c2ccccc12 ; Cc1c(N(c2ccccc2)c2cc(N(c3ccccc3)c3c(C)n(-c4ccccc4)c4ccccc34)c3ccc4c(N(c5ccccc5)c5c(C)n(-c6ccccc6)c6ccccc56)cc(N(c5ccccc5)c5c(C)n(-c6ccccc6)c6ccccc56)c5ccc2c3c54)c2ccccc2n1-c1ccccc1 ; Cc1cc2ccccc2n1N(c1ccccc1)c1cc(N(c2ccccc2)n2c(C)cc3ccccc32)c2ccc3c(N(c4ccccc4)n4c(C)cc5ccccc54)cc(N(c4ccccc4)n4c(C)cc5ccccc54)c4ccc1c2c43]

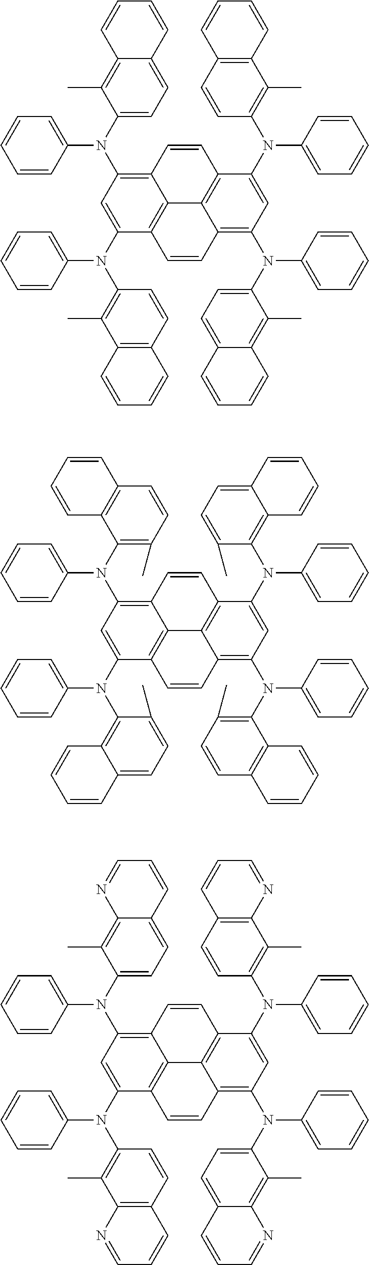 [SMILES: Cc1c(N(c2ccccc2)c2cc(N(c3ccccc3)c3ccc4ccccc4c3C)c3ccc4c(N(c5ccccc5)c5ccc6ccccc6c5C)cc(N(c5ccccc5)c5ccc6ccccc6c5C)c5ccc2c3c54)ccc2ccccc12 ; Cc1ccc2ccccc2c1N(c1ccccc1)c1cc(N(c2ccccc2)c2c(C)ccc3ccccc23)c2ccc3c(N(c4ccccc4)c4c(C)ccc5ccccc45)cc(N(c4ccccc4)c4c(C)ccc5ccccc45)c4ccc1c2c43 ; Cc1c(N(c2ccccc2)c2cc(N(c3ccccc3)c3ccc4cccnc4c3C)c3ccc4c(N(c5ccccc5)c5ccc6cccnc6c5C)cc(N(c5ccccc5)c5ccc6cccnc6c5C)c5ccc2c3c54)ccc2cccnc12]

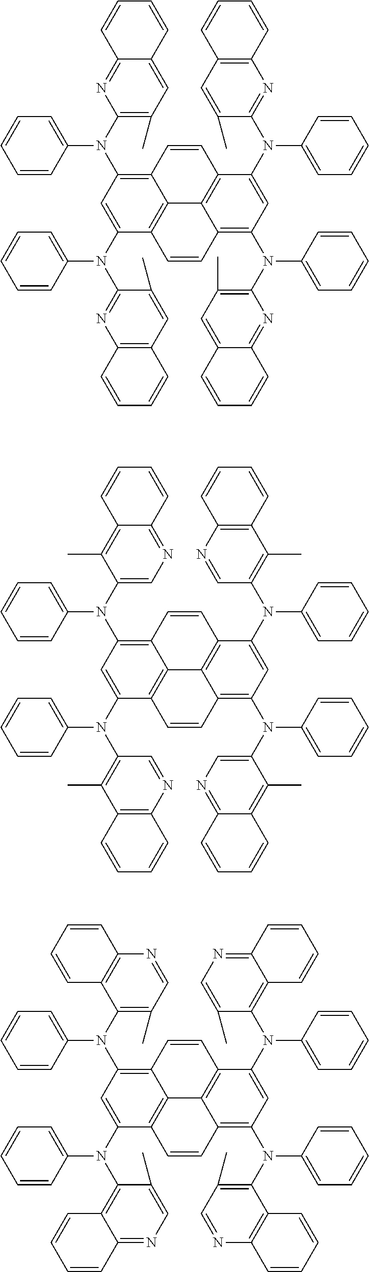 [SMILES: Cc1cc2ccccc2nc1N(c1ccccc1)c1cc(N(c2ccccc2)c2nc3ccccc3cc2C)c2ccc3c(N(c4ccccc4)c4nc5ccccc5cc4C)cc(N(c4ccccc4)c4nc5ccccc5cc4C)c4ccc1c2c43 ; Cc1c(N(c2ccccc2)c2cc(N(c3ccccc3)c3cnc4ccccc4c3C)c3ccc4c(N(c5ccccc5)c5cnc6ccccc6c5C)cc(N(c5ccccc5)c5cnc6ccccc6c5C)c5ccc2c3c54)cnc2ccccc12 ; Cc1cnc2ccccc2c1N(c1ccccc1)c1cc(N(c2ccccc2)c2c(C)cnc3ccccc23)c2ccc3c(N(c4ccccc4)c4c(C)cnc5ccccc45)cc(N(c4ccccc4)c4c(C)cnc5ccccc45)c4ccc1c2c43]

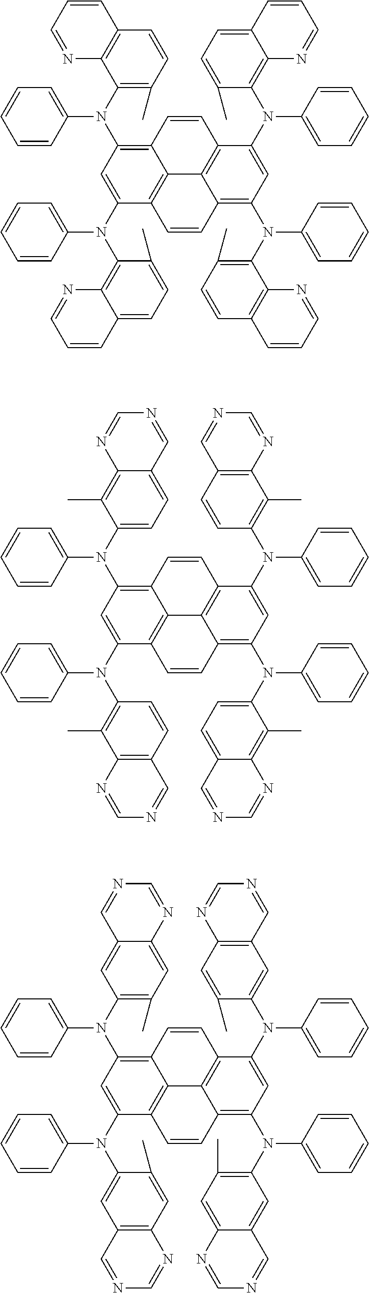 [SMILES: Cc1ccc2cccnc2c1N(c1ccccc1)c1cc(N(c2ccccc2)c2c(C)ccc3cccnc23)c2ccc3c(N(c4ccccc4)c4c(C)ccc5cccnc45)cc(N(c4ccccc4)c4c(C)ccc5cccnc45)c4ccc1c2c43 ; Cc1c(N(c2ccccc2)c2cc(N(c3ccccc3)c3ccc4cncnc4c3C)c3ccc4c(N(c5ccccc5)c5ccc6cncnc6c5C)cc(N(c5ccccc5)c5ccc6cncnc6c5C)c5ccc2c3c54)ccc2cncnc12 ; Cc1cc2ncncc2cc1N(c1ccccc1)c1cc(N(c2ccccc2)c2cc3cncnc3cc2C)c2ccc3c(N(c4ccccc4)c4cc5cncnc5cc4C)cc(N(c4ccccc4)c4cc5cncnc5cc4C)c4ccc1c2c43]

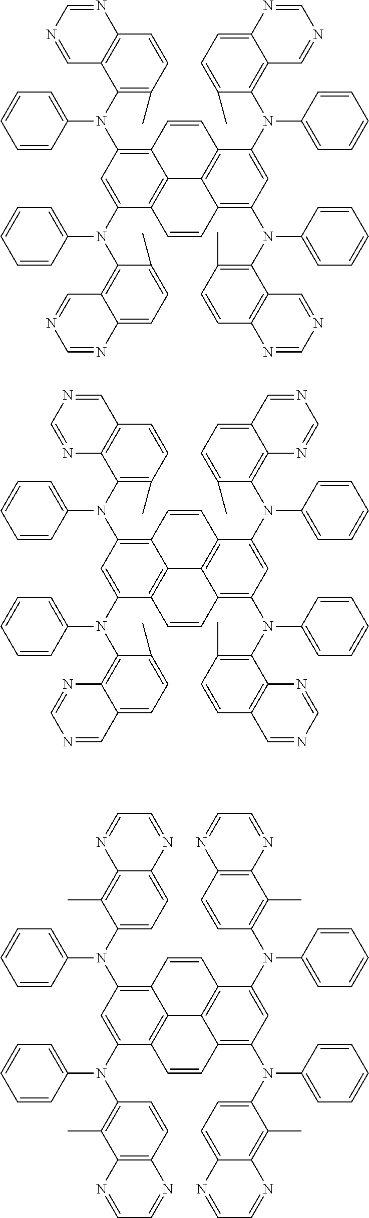

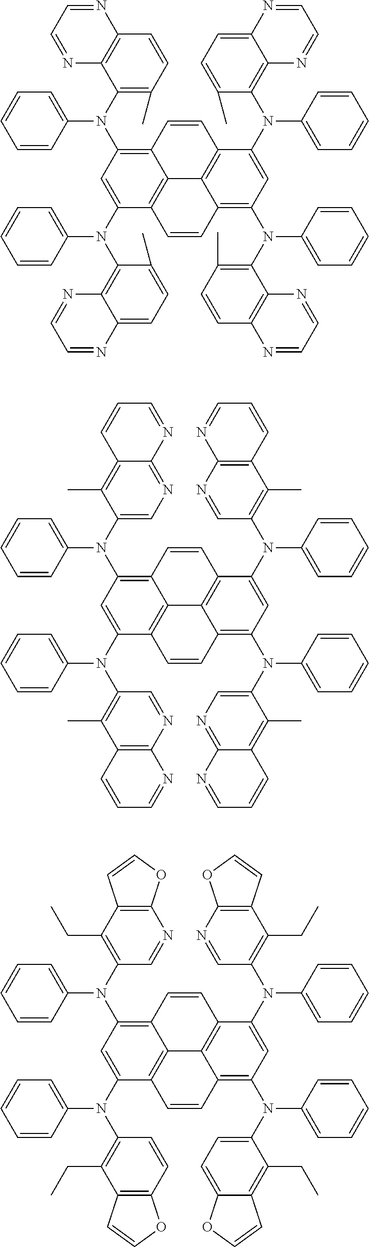 [SMILES: Cc1ccc2nccnc2c1N(c1ccccc1)c1cc(N(c2ccccc2)c2c(C)ccc3nccnc23)c2ccc3c(N(c4ccccc4)c4c(C)ccc5nccnc45)cc(N(c4ccccc4)c4c(C)ccc5nccnc45)c4ccc1c2c43 ; Cc1c(N(c2ccccc2)c2cc(N(c3ccccc3)c3cnc4ncccc4c3C)c3ccc4c(N(c5ccccc5)c5cnc6ncccc6c5C)cc(N(c5ccccc5)c5cnc6ncccc6c5C)c5ccc2c3c54)cnc2ncccc12 ; CCc1c(N(c2ccccc2)c2cc(N(c3ccccc3)c3cnc4occc4c3CC)c3ccc4c(N(c5ccccc5)c5cnc6occc6c5CC)cc(N(c5ccccc5)c5ccc6occc6c5CC)c5ccc2c3c54)ccc2occc12]

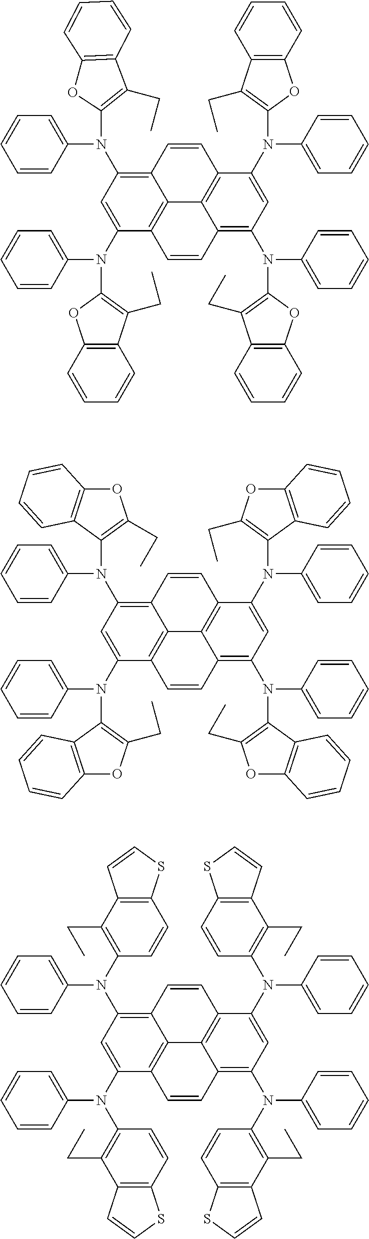 [SMILES: CCc1c(N(c2ccccc2)c2cc(N(c3ccccc3)c3oc4ccccc4c3CC)c3ccc4c(N(c5ccccc5)c5oc6ccccc6c5CC)cc(N(c5ccccc5)c5oc6ccccc6c5CC)c5ccc2c3c54)oc2ccccc12 ; CCc1oc2ccccc2c1N(c1ccccc1)c1cc(N(c2ccccc2)c2c(CC)oc3ccccc23)c2ccc3c(N(c4ccccc4)c4c(CC)oc5ccccc45)cc(N(c4ccccc4)c4c(CC)oc5ccccc45)c4ccc1c2c43 ; CCc1c(N(c2ccccc2)c2cc(N(c3ccccc3)c3ccc4sccc4c3CC)c3ccc4c(N(c5ccccc5)c5ccc6sccc6c5CC)cc(N(c5ccccc5)c5ccc6sccc6c5CC)c5ccc2c3c54)ccc2sccc12]

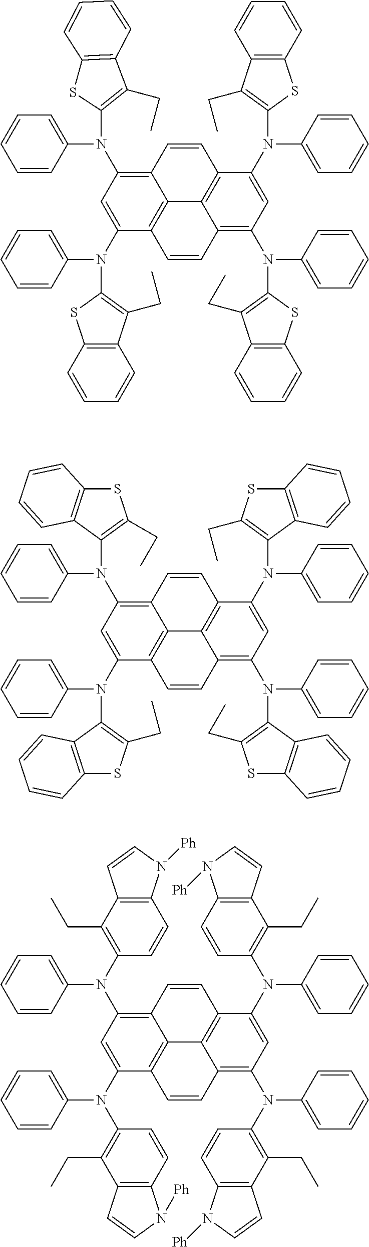 [SMILES: CCc1c(N(c2ccccc2)c2cc(N(c3ccccc3)c3sc4ccccc4c3CC)c3ccc4c(N(c5ccccc5)c5sc6ccccc6c5CC)cc(N(c5ccccc5)c5sc6ccccc6c5CC)c5ccc2c3c54)sc2ccccc12 ; CCc1sc2ccccc2c1N(c1ccccc1)c1cc(N(c2ccccc2)c2c(CC)sc3ccccc23)c2ccc3c(N(c4ccccc4)c4c(CC)sc5ccccc45)cc(N(c4ccccc4)c4c(CC)sc5ccccc45)c4ccc1c2c43 ; CCc1c(N(c2ccccc2)c2cc(N(c3ccccc3)c3ccc4c(ccn4-c4ccccc4)c3CC)c3ccc4c(N(c5ccccc5)c5ccc6c(ccn6-c6ccccc6)c5CC)cc(N(c5ccccc5)c5ccc6c(ccn6-c6ccccc6)c5CC)c5ccc2c3c54)ccc2c1ccn2-c1ccccc1]

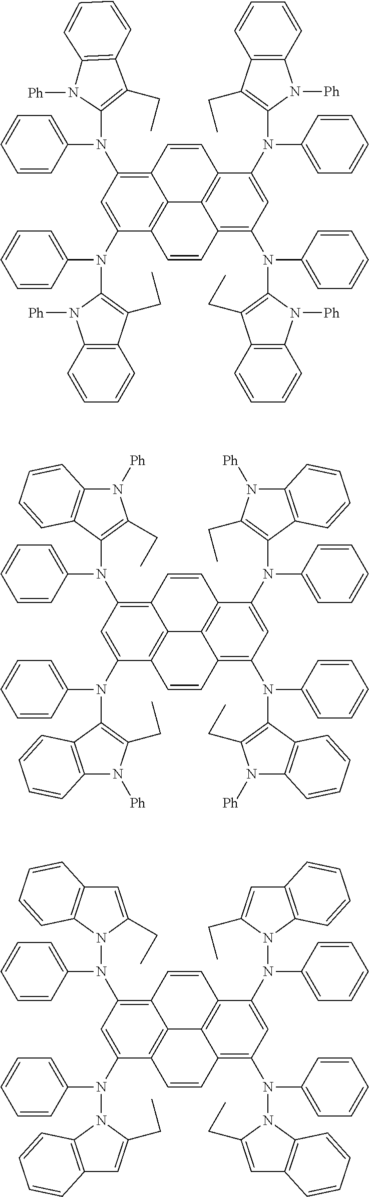

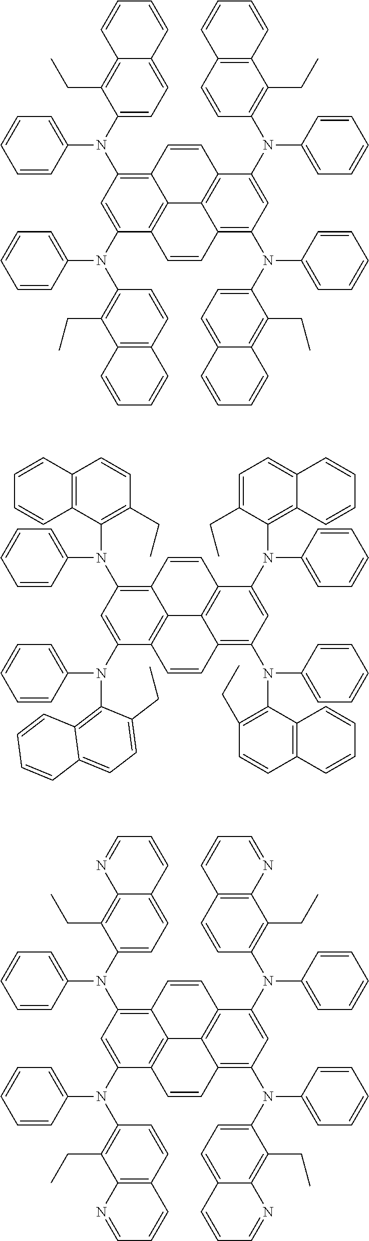 [SMILES: CCc1c(N(c2ccccc2)c2cc(N(c3ccccc3)c3ccc4ccccc4c3CC)c3ccc4c(N(c5ccccc5)c5ccc6ccccc6c5CC)cc(N(c5ccccc5)c5ccc6ccccc6c5CC)c5ccc2c3c54)ccc2ccccc12 ; CCc1ccc2ccccc2c1N(c1ccccc1)c1cc(N(c2ccccc2)c2c(CC)ccc3ccccc23)c2ccc3c(N(c4ccccc4)c4c(CC)ccc5ccccc45)cc(N(c4ccccc4)c4c(CC)ccc5ccccc45)c4ccc1c2c43 ; CCc1c(N(c2ccccc2)c2cc(N(c3ccccc3)c3ccc4cccnc4c3CC)c3ccc4c(N(c5ccccc5)c5ccc6cccnc6c5CC)cc(N(c5ccccc5)c5ccc6cccnc6c5CC)c5ccc2c3c54)ccc2cccnc12]

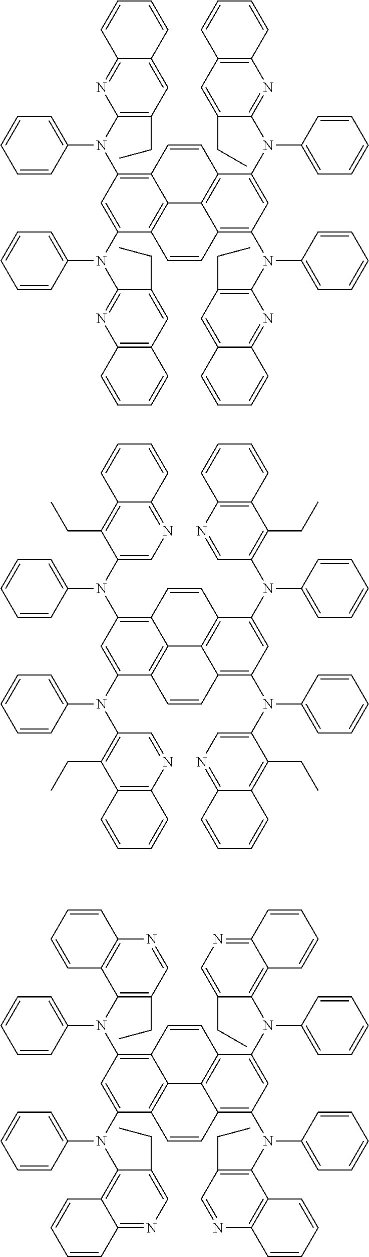

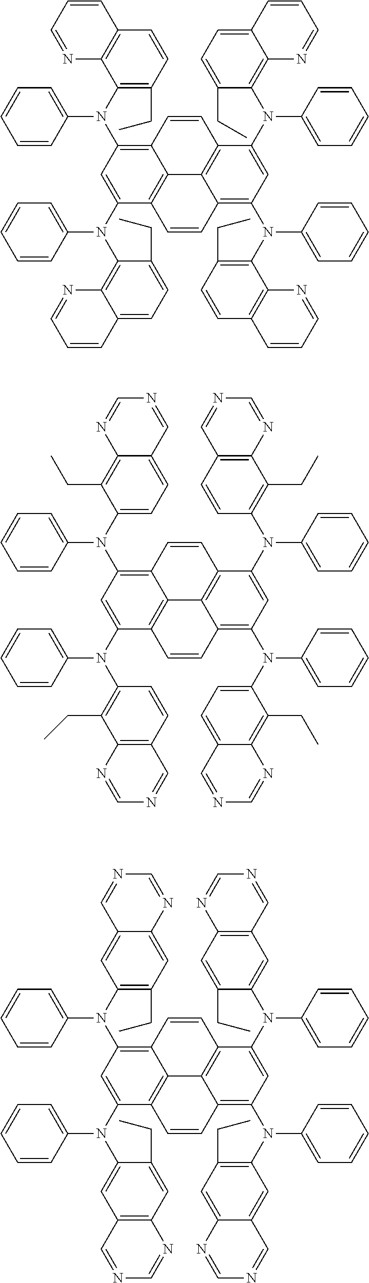 [SMILES: CCc1ccc2cccnc2c1N(c1ccccc1)c1cc(N(c2ccccc2)c2c(CC)ccc3cccnc23)c2ccc3c(N(c4ccccc4)c4c(CC)ccc5cccnc45)cc(N(c4ccccc4)c4c(CC)ccc5cccnc45)c4ccc1c2c43 ; CCc1c(N(c2ccccc2)c2cc(N(c3ccccc3)c3ccc4cncnc4c3CC)c3ccc4c(N(c5ccccc5)c5ccc6cncnc6c5CC)cc(N(c5ccccc5)c5ccc6cncnc6c5CC)c5ccc2c3c54)ccc2cncnc12 ; CCc1cc2ncncc2cc1N(c1ccccc1)c1cc(N(c2ccccc2)c2cc3cncnc3cc2CC)c2ccc3c(N(c4ccccc4)c4cc5cncnc5cc4CC)cc(N(c4ccccc4)c4cc5cncnc5cc4CC)c4ccc1c2c43]

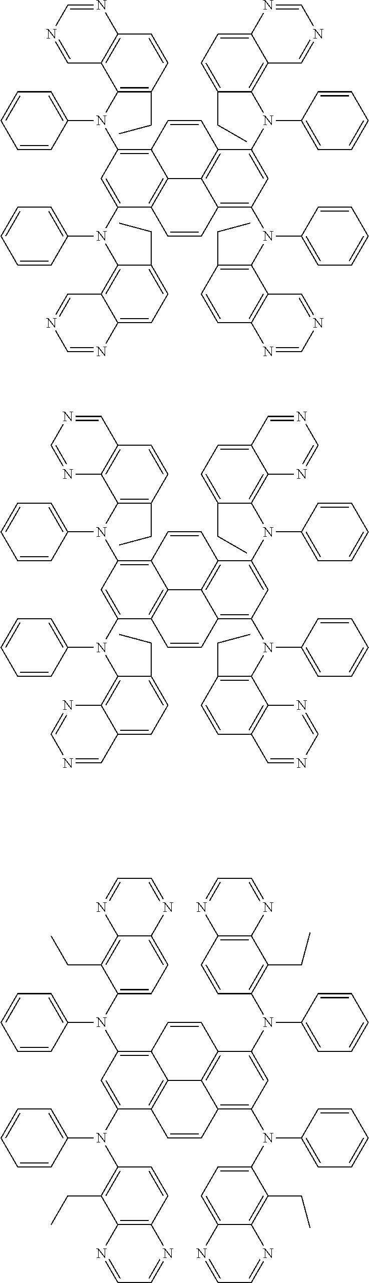 [SMILES: CCc1ccc2ncncc2c1N(c1ccccc1)c1cc(N(c2ccccc2)c2c(CC)ccc3ncncc23)c2ccc3c(N(c4ccccc4)c4c(CC)ccc5ncncc45)cc(N(c4ccccc4)c4c(CC)ccc5ncncc45)c4ccc1c2c43 ; CCc1ccc2cncnc2c1N(c1ccccc1)c1cc(N(c2ccccc2)c2c(CC)ccc3cncnc23)c2ccc3c(N(c4ccccc4)c4c(CC)ccc5cncnc45)cc(N(c4ccccc4)c4c(CC)ccc5cncnc45)c4ccc1c2c43 ; CCc1c(N(c2ccccc2)c2cc(N(c3ccccc3)c3ccc4nccnc4c3CC)c3ccc4c(N(c5ccccc5)c5ccc6nccnc6c5CC)cc(N(c5ccccc5)c5ccc6nccnc6c5CC)c5ccc2c3c54)ccc2nccnc12]

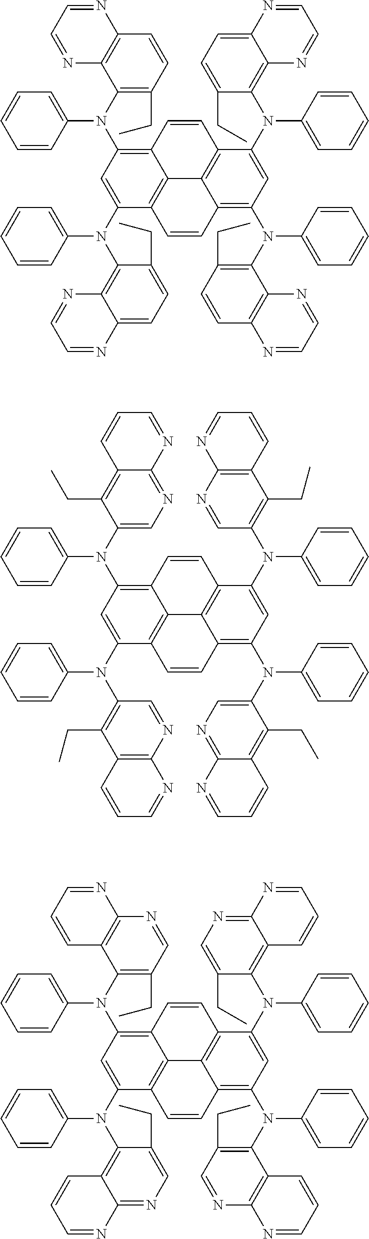 [SMILES: CCc1ccc2nccnc2c1N(c1ccccc1)c1cc(N(c2ccccc2)c2c(CC)ccc3nccnc23)c2ccc3c(N(c4ccccc4)c4c(CC)ccc5nccnc45)cc(N(c4ccccc4)c4c(CC)ccc5nccnc45)c4ccc1c2c43 ; CCc1c(N(c2ccccc2)c2cc(N(c3ccccc3)c3cnc4ncccc4c3CC)c3ccc4c(N(c5ccccc5)c5cnc6ncccc6c5CC)cc(N(c5ccccc5)c5cnc6ncccc6c5CC)c5ccc2c3c54)cnc2ncccc12 ; CCc1cnc2ncccc2c1N(c1ccccc1)c1cc(N(c2ccccc2)c2c(CC)cnc3ncccc23)c2ccc3c(N(c4ccccc4)c4c(CC)cnc5ncccc45)cc(N(c4ccccc4)c4c(CC)cnc5ncccc45)c4ccc1c2c43]

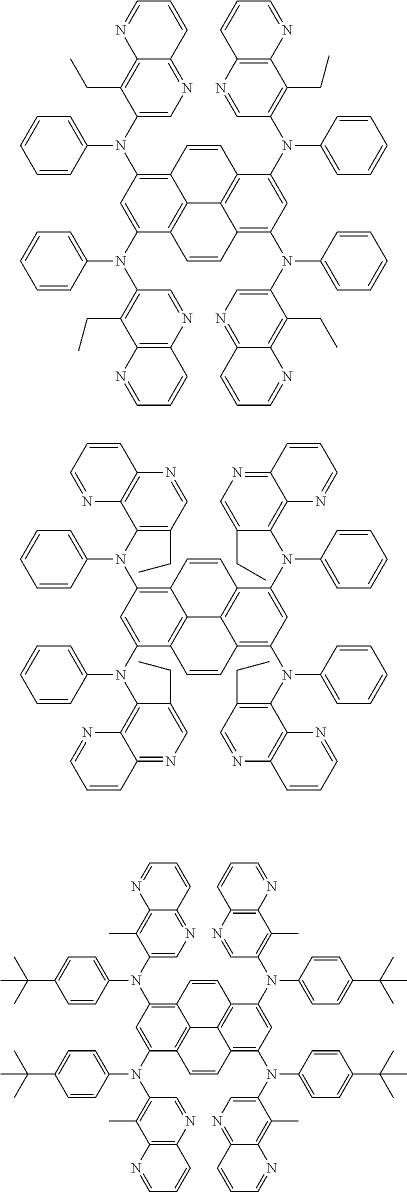

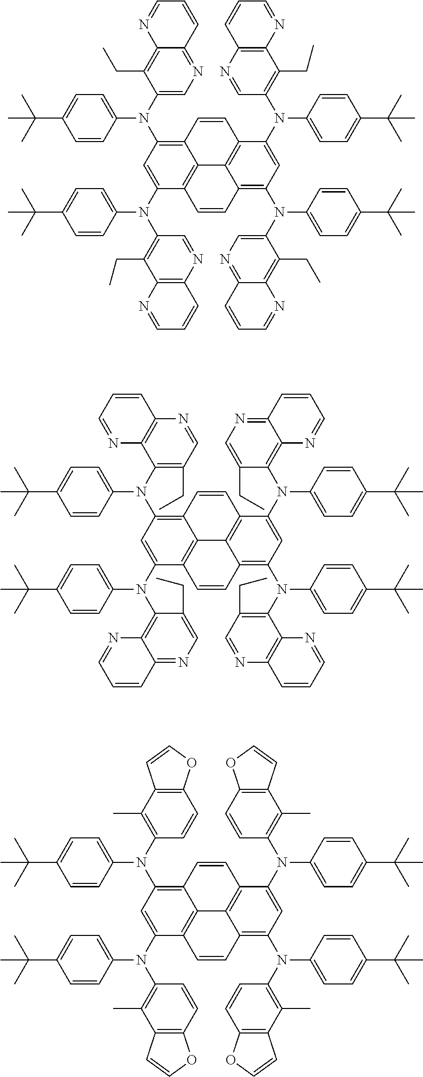

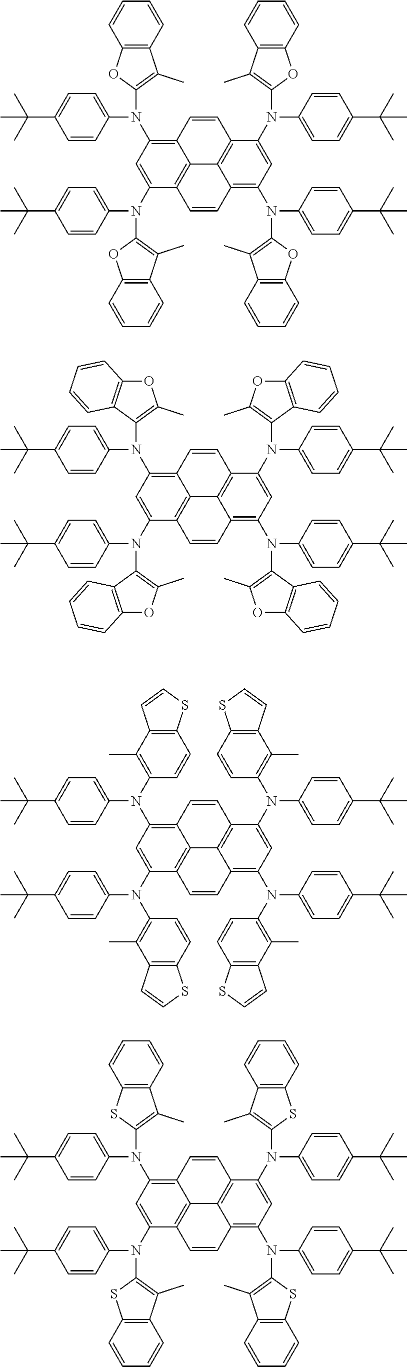

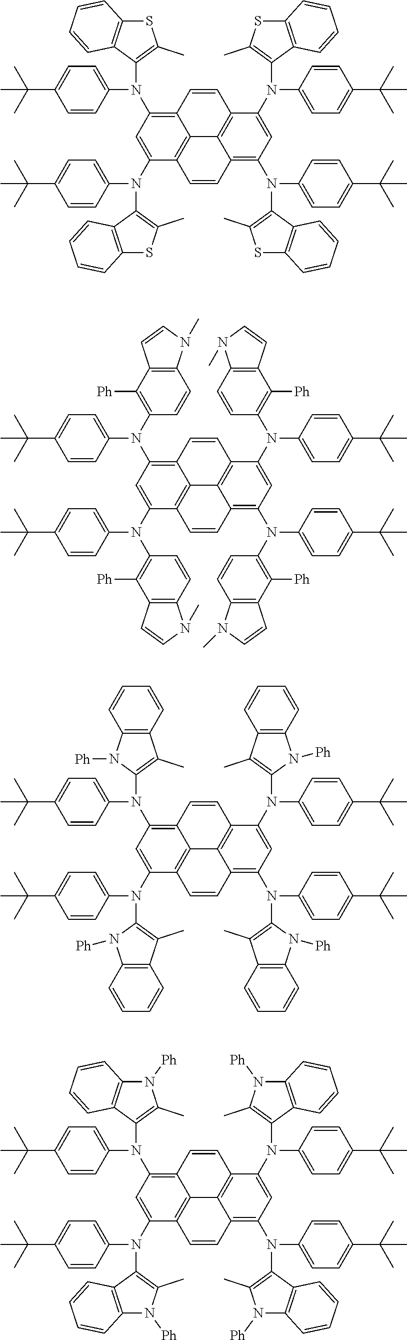

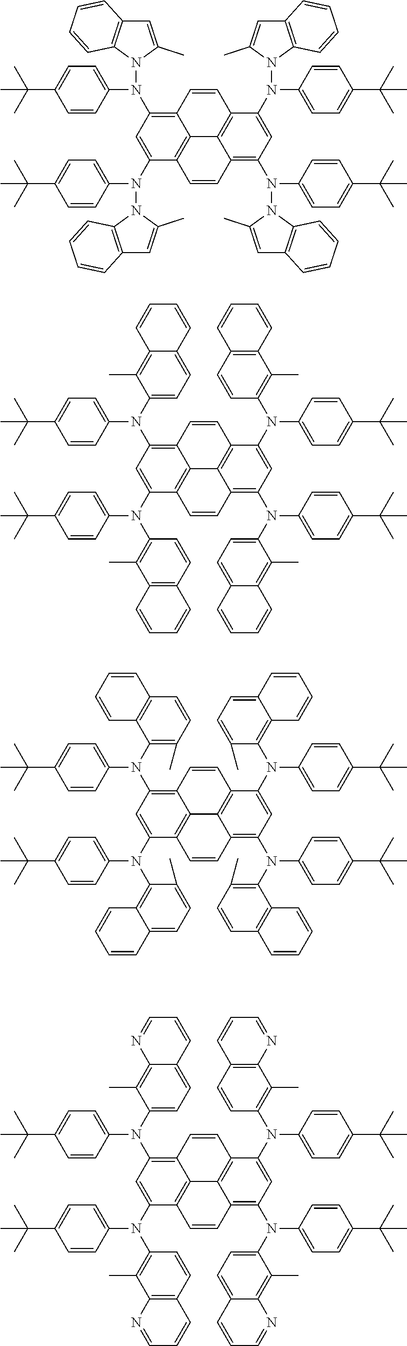

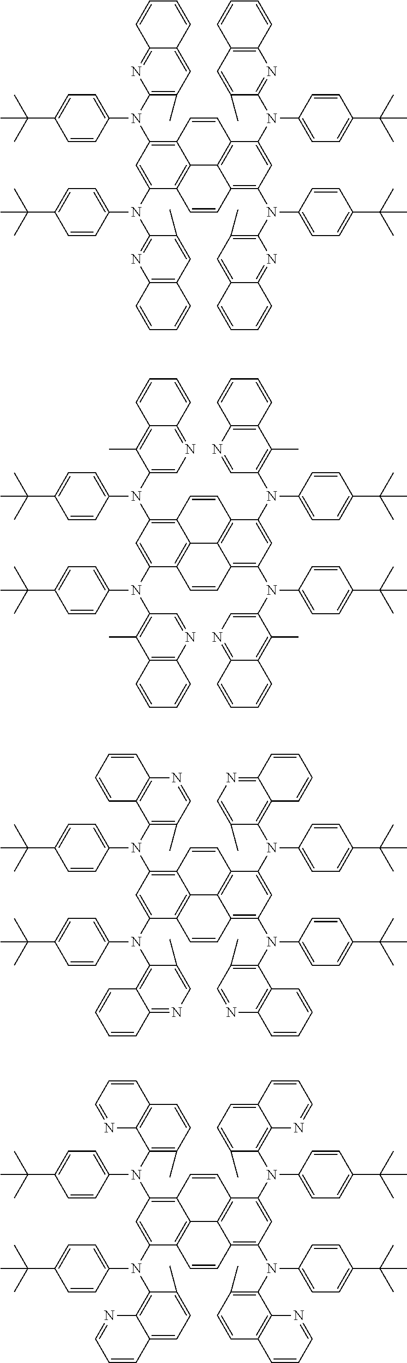

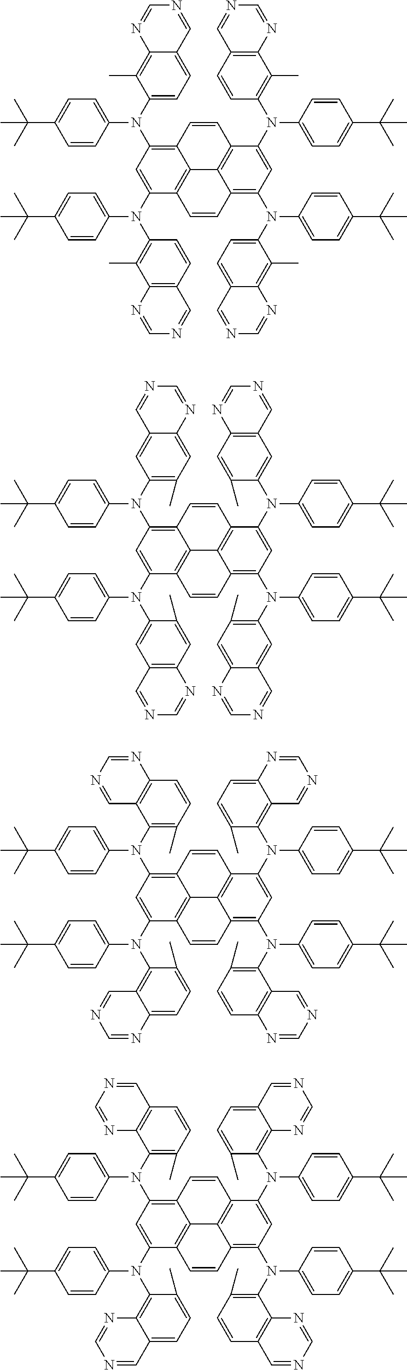

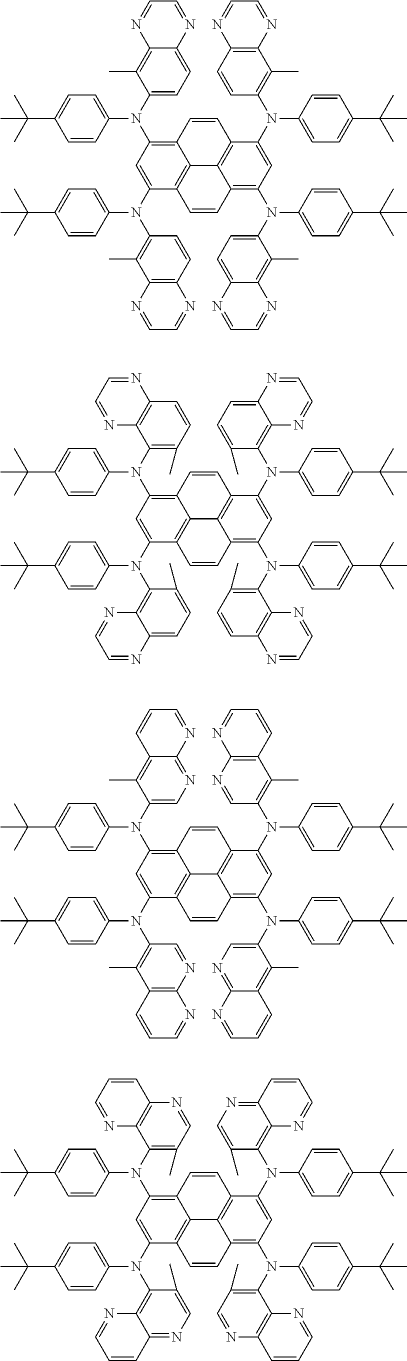

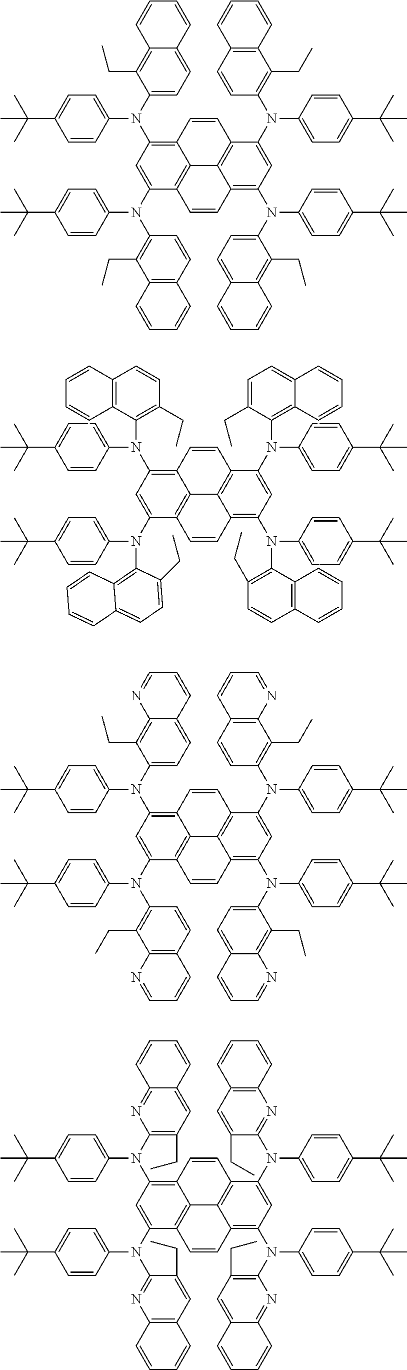

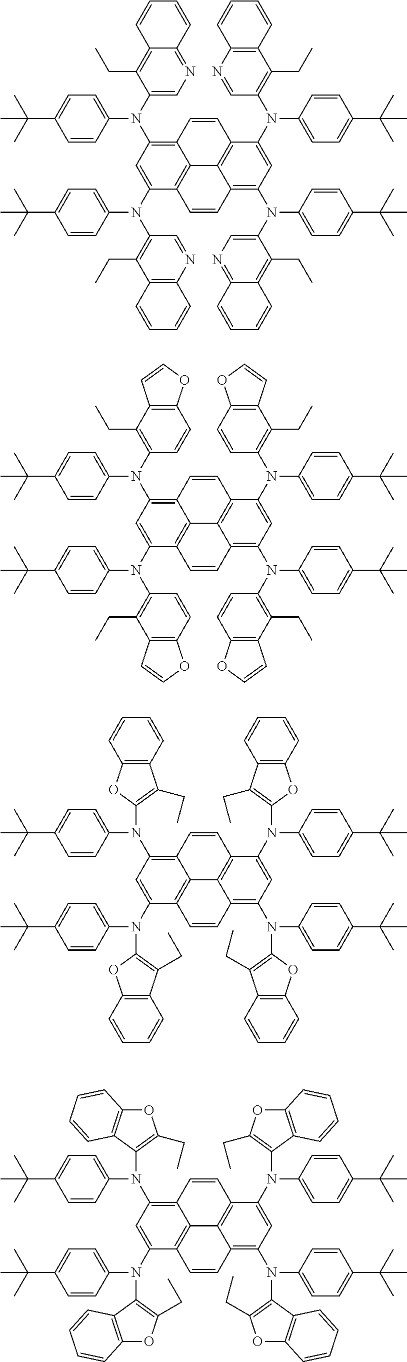

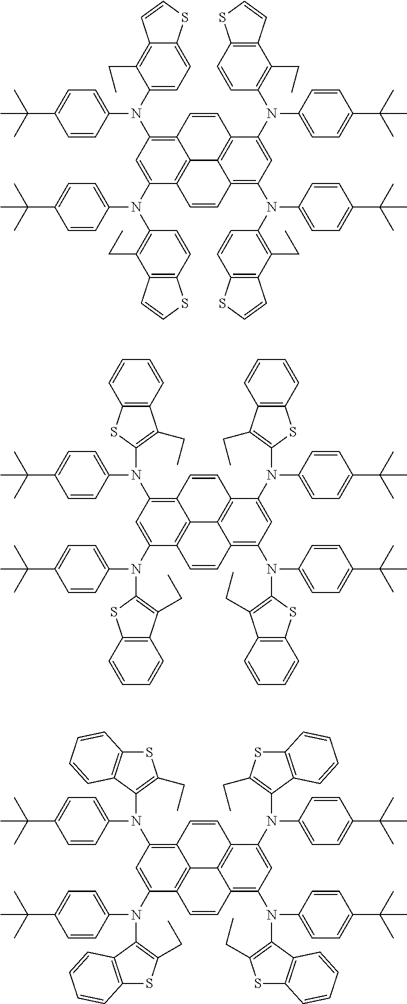

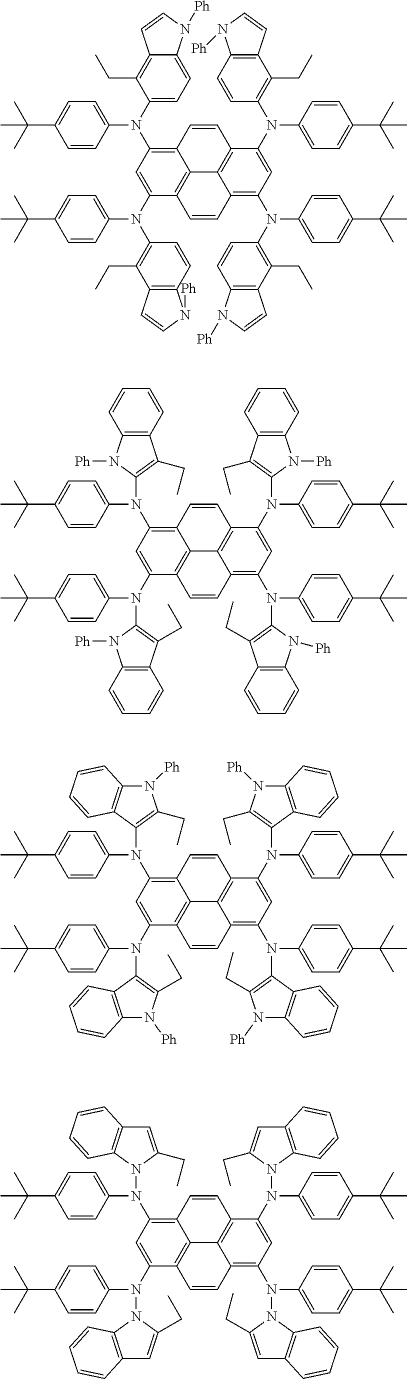

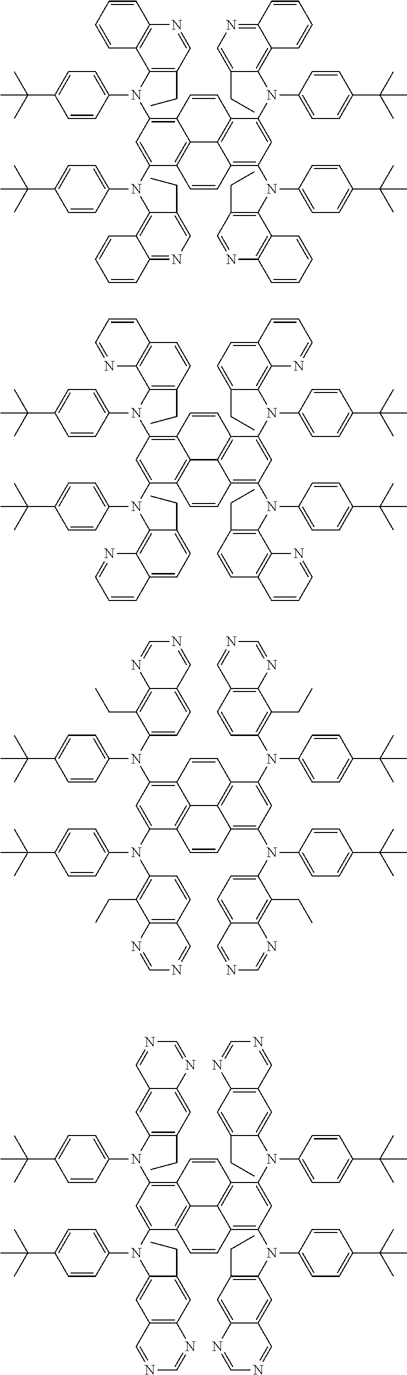

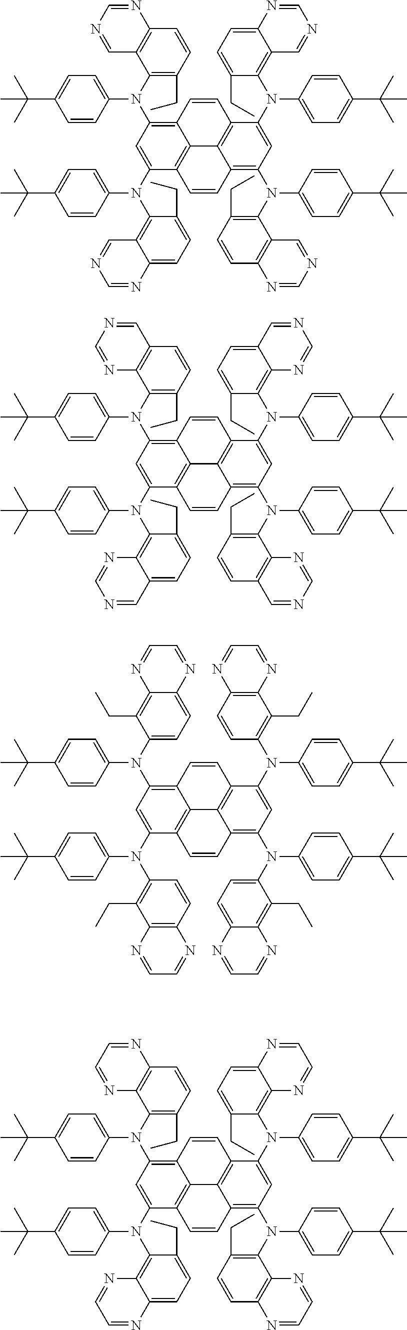

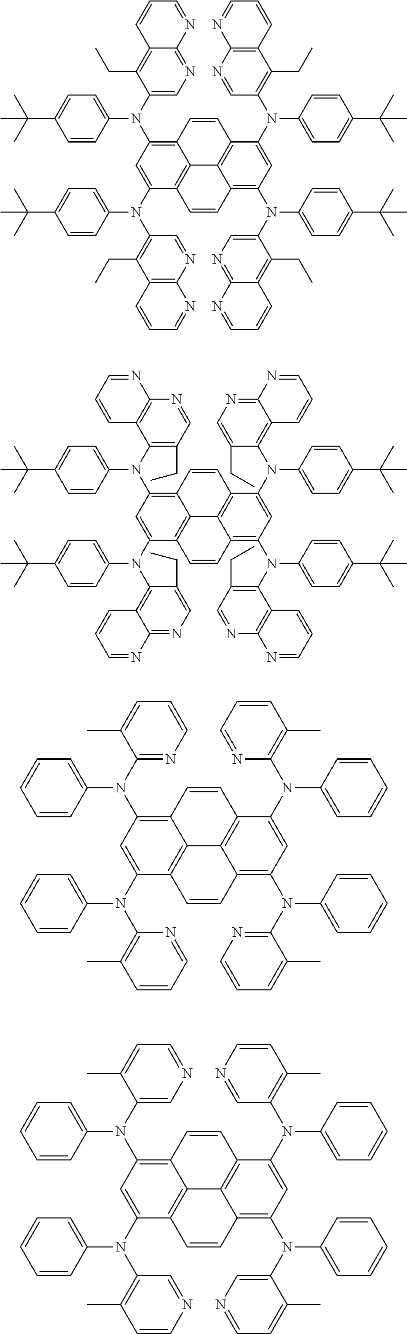

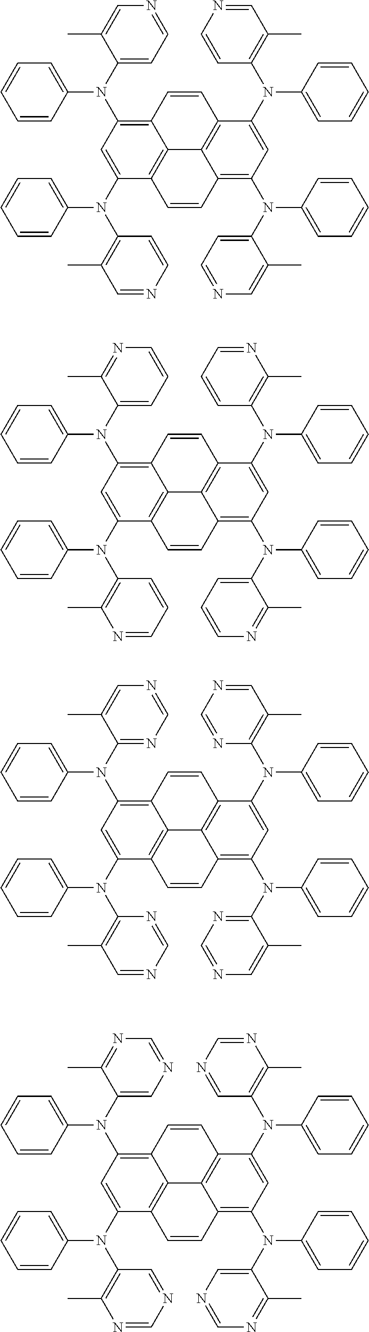 [SMILES: Cc1cnccc1N(c1ccccc1)c1cc(N(c2ccccc2)c2ccncc2C)c2ccc3c(N(c4ccccc4)c4ccncc4C)cc(N(c4ccccc4)c4ccncc4C)c4ccc1c2c43 ; Cc1ncccc1N(c1ccccc1)c1cc(N(c2ccccc2)c2cccnc2C)c2ccc3c(N(c4ccccc4)c4cccnc4C)cc(N(c4ccccc4)c4cccnc4C)c4ccc1c2c43 ; Cc1cncnc1N(c1ccccc1)c1cc(N(c2ccccc2)c2ncncc2C)c2ccc3c(N(c4ccccc4)c4ncncc4C)cc(N(c4ccccc4)c4ncncc4C)c4ccc1c2c43 ; Cc1ncncc1N(c1ccccc1)c1cc(N(c2ccccc2)c2cncnc2C)c2ccc3c(N(c4ccccc4)c4cncnc4C)cc(N(c4ccccc4)c4cncnc4C)c4ccc1c2c43]

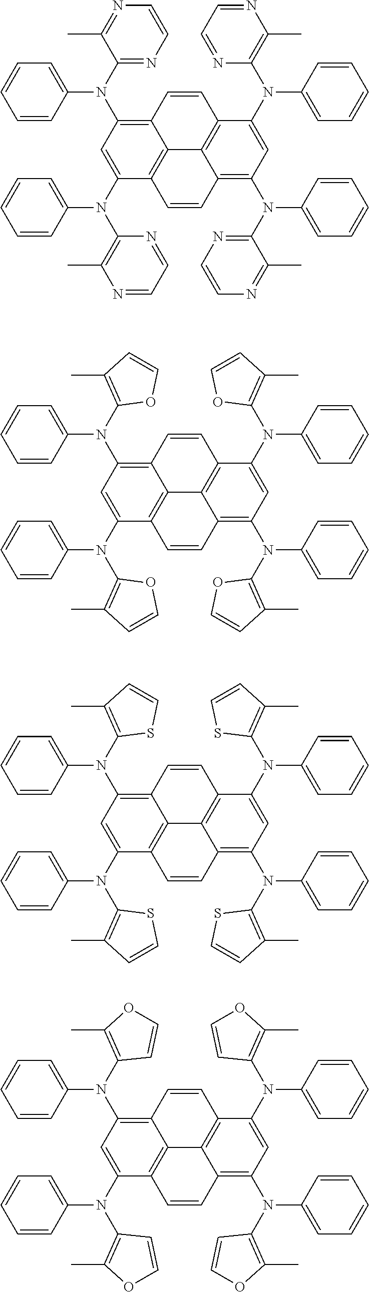

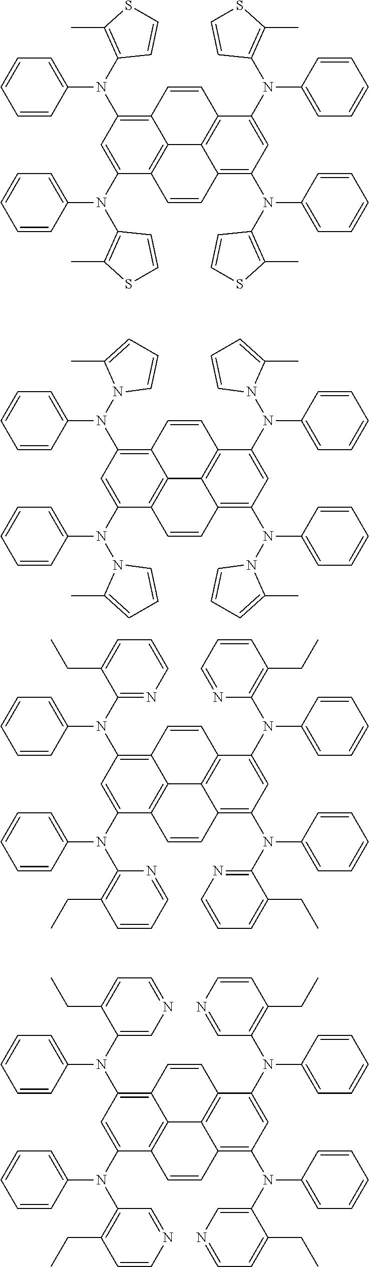

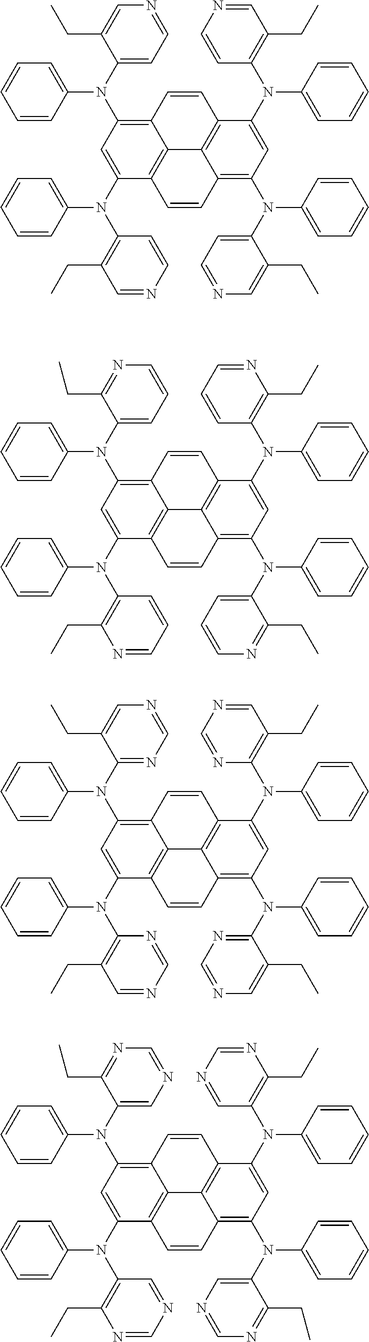 [SMILES: CCc1cnccc1N(c1ccccc1)c1cc(N(c2ccccc2)c2ccncc2CC)c2ccc3c(N(c4ccccc4)c4ccncc4CC)cc(N(c4ccccc4)c4ccncc4CC)c4ccc1c2c43 ; CCc1ncccc1N(c1ccccc1)c1cc(N(c2ccccc2)c2cccnc2CC)c2ccc3c(N(c4ccccc4)c4cccnc4CC)cc(N(c4ccccc4)c4cccnc4CC)c4ccc1c2c43 ; CCc1cncnc1N(c1ccccc1)c1cc(N(c2ccccc2)c2ncncc2CC)c2ccc3c(N(c4ccccc4)c4ncncc4CC)cc(N(c4ccccc4)c4ncncc4CC)c4ccc1c2c43 ; CCc1ncncc1N(c1ccccc1)c1cc(N(c2ccccc2)c2cncnc2CC)c2ccc3c(N(c4ccccc4)c4cncnc4CC)cc(N(c4ccccc4)c4cncnc4CC)c4ccc1c2c43]

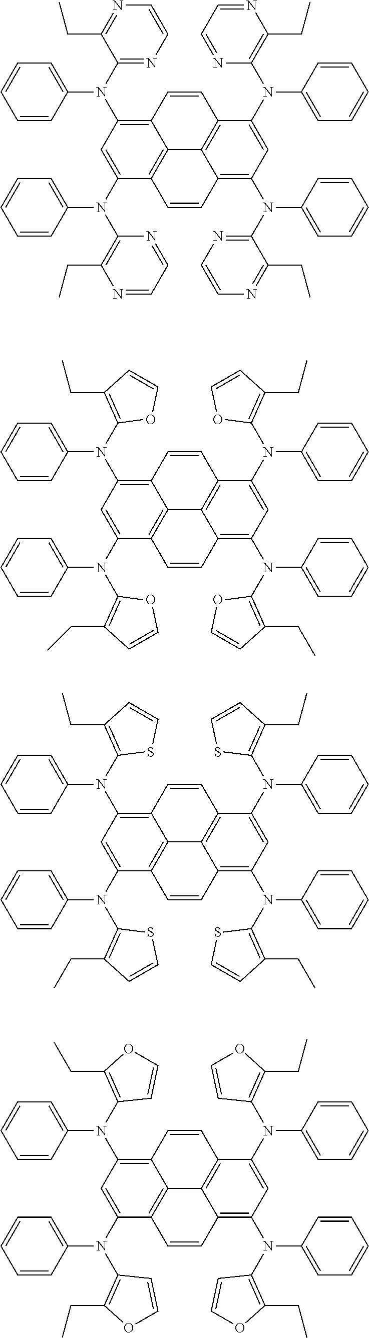

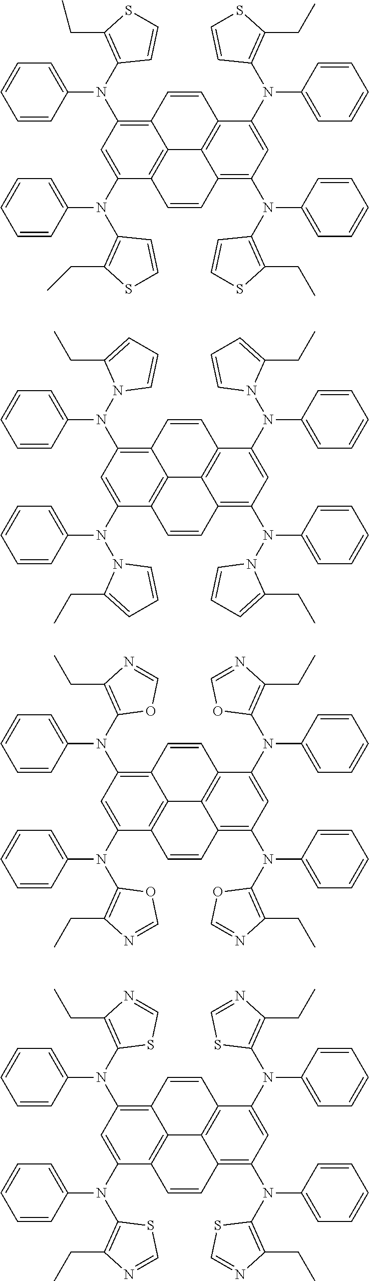

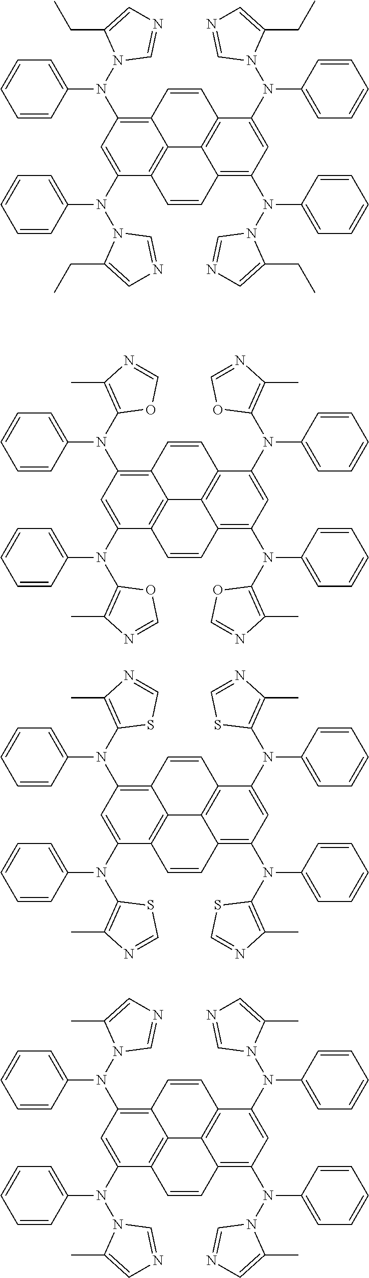

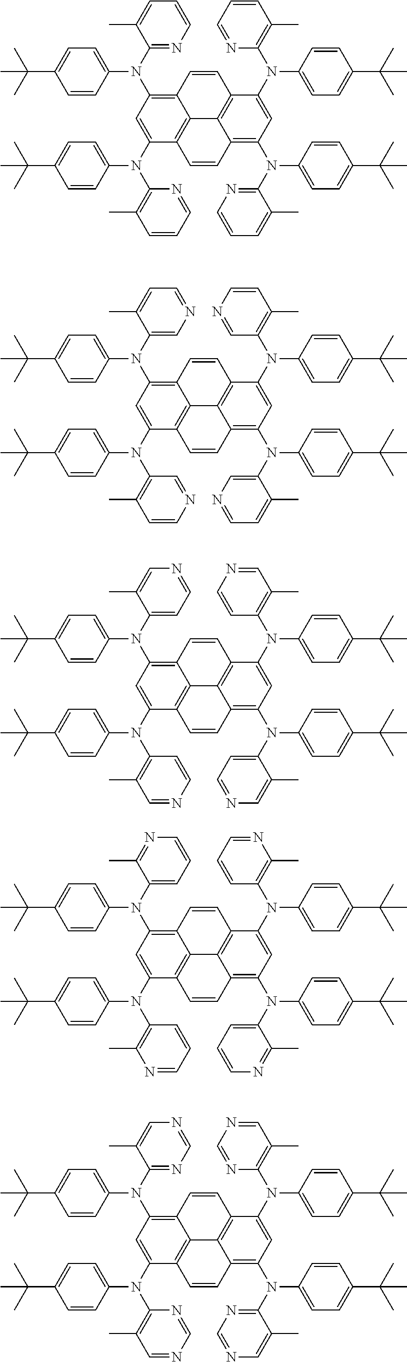

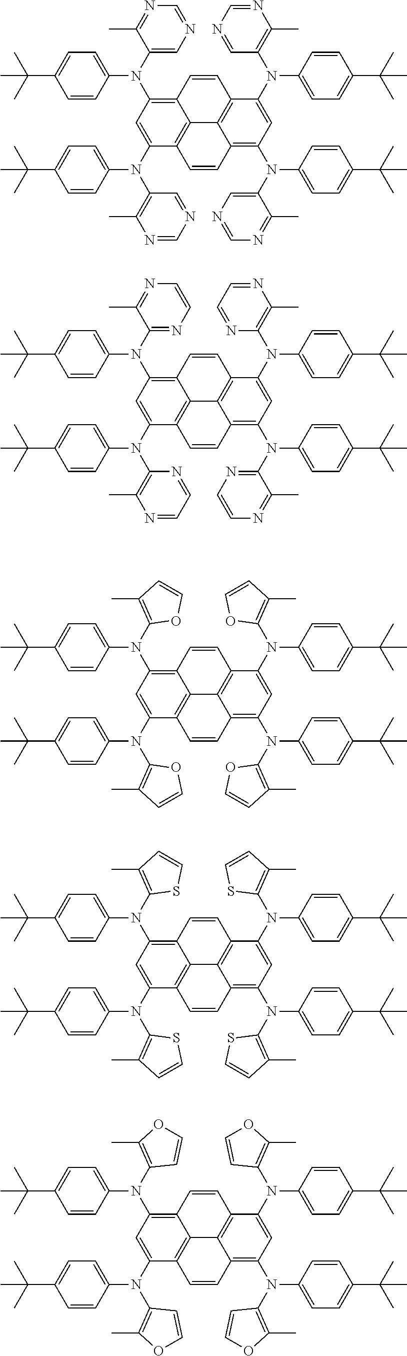

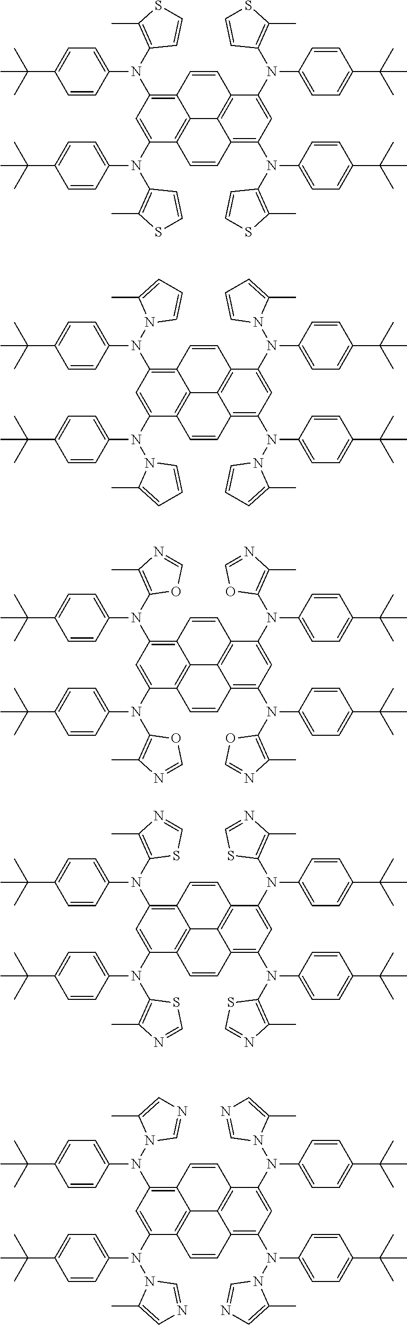

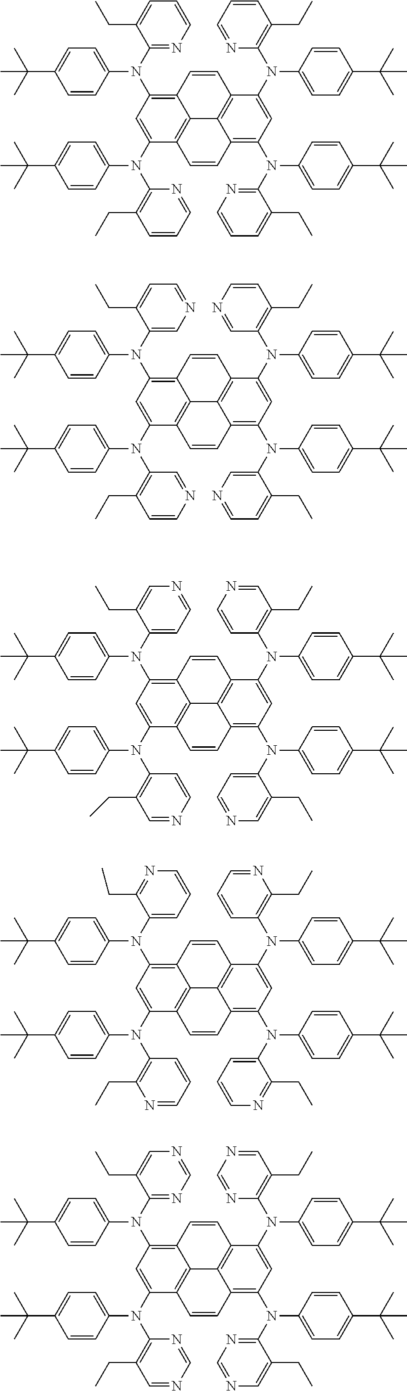

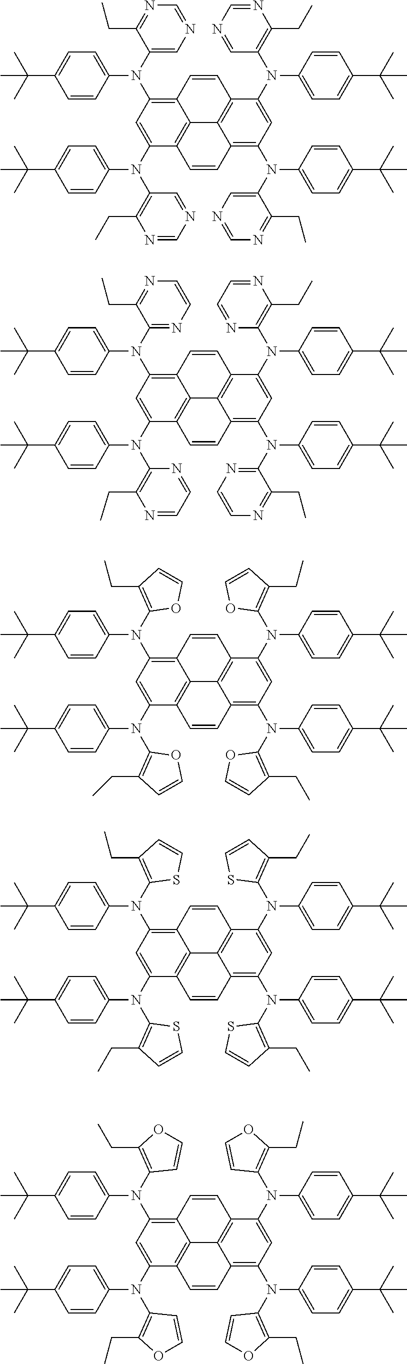

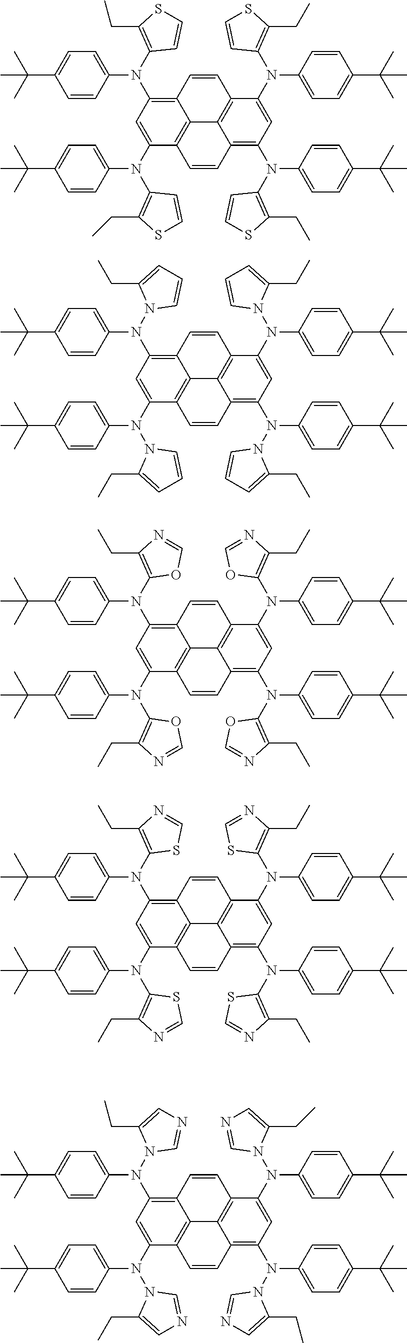

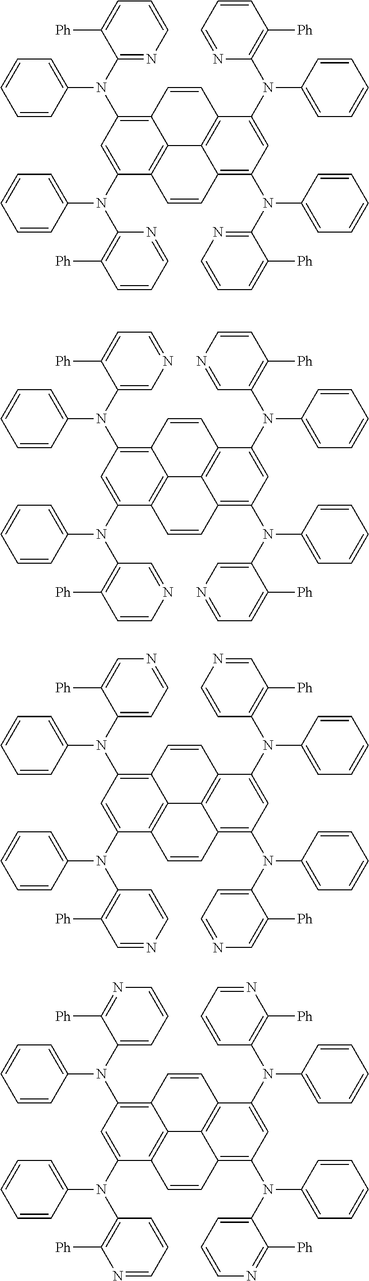

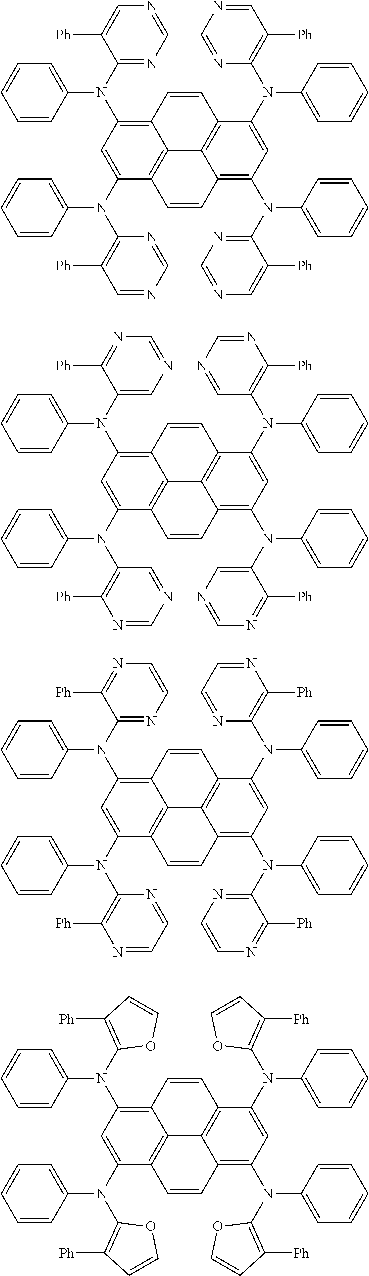

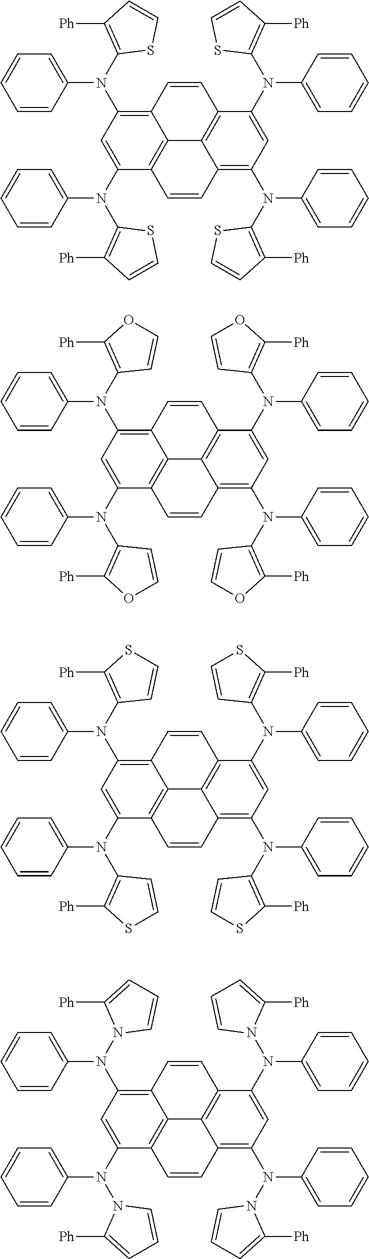

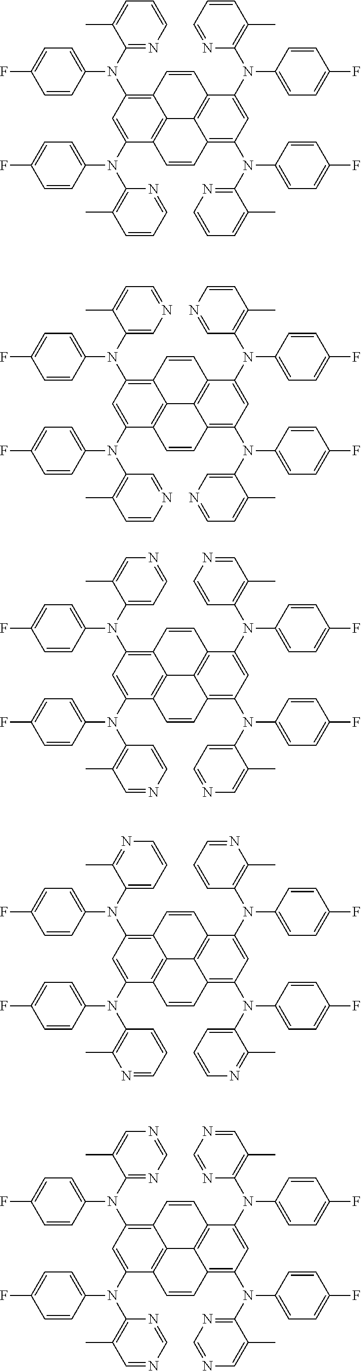

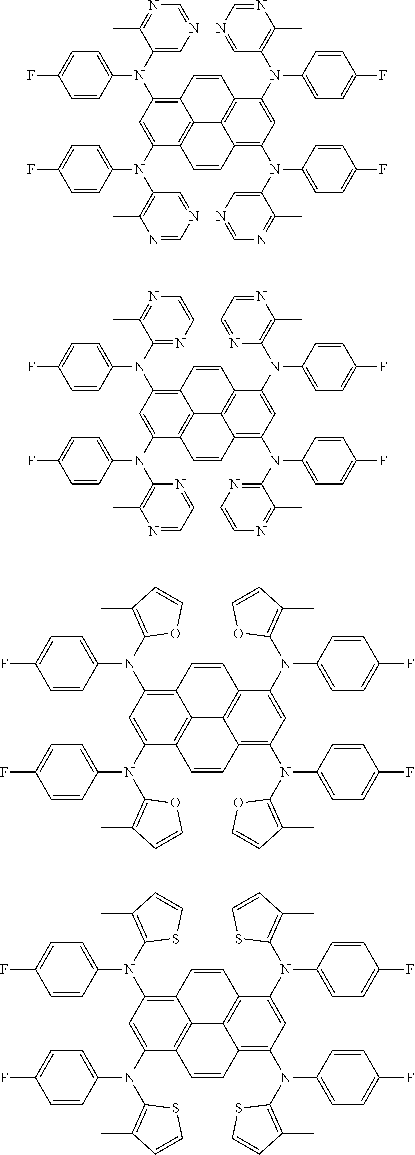 [SMILES: Cc1ncncc1N(c1ccc(F)cc1)c1cc(N(c2ccc(F)cc2)c2cncnc2C)c2ccc3c(N(c4ccc(F)cc4)c4cncnc4C)cc(N(c4ccc(F)cc4)c4cncnc4C)c4ccc1c2c43 ; Cc1nccnc1N(c1ccc(F)cc1)c1cc(N(c2ccc(F)cc2)c2nccnc2C)c2ccc3c(N(c4ccc(F)cc4)c4nccnc4C)cc(N(c4ccc(F)cc4)c4nccnc4C)c4ccc1c2c43 ; Cc1ccoc1N(c1ccc(F)cc1)c1cc(N(c2ccc(F)cc2)c2occc2C)c2ccc3c(N(c4ccc(F)cc4)c4occc4C)cc(N(c4ccc(F)cc4)c4occc4C)c4ccc1c2c43 ; Cc1ccsc1N(c1ccc(F)cc1)c1cc(N(c2ccc(F)cc2)c2sccc2C)c2ccc3c(N(c4ccc(F)cc4)c4sccc4C)cc(N(c4ccc(F)cc4)c4sccc4C)c4ccc1c2c43]

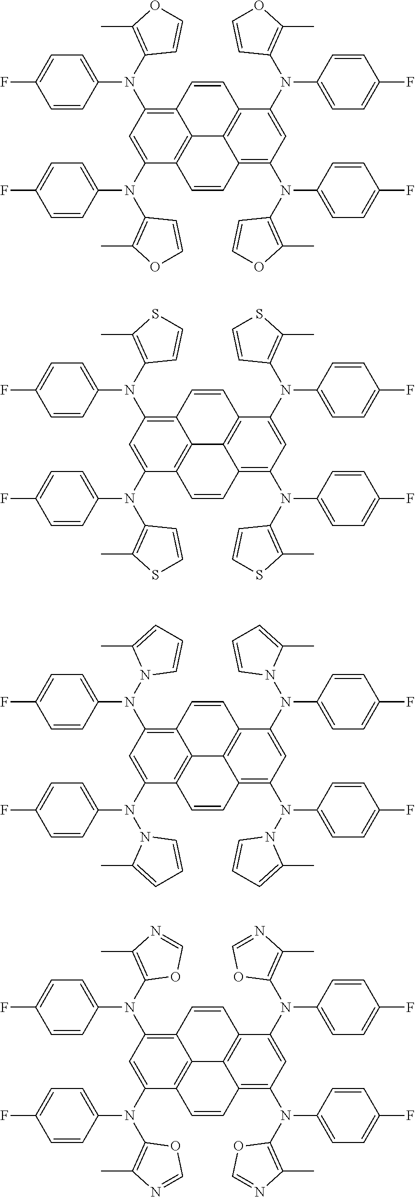 [SMILES: Cc1occc1N(c1ccc(F)cc1)c1cc(N(c2ccc(F)cc2)c2ccoc2C)c2ccc3c(N(c4ccc(F)cc4)c4ccoc4C)cc(N(c4ccc(F)cc4)c4ccoc4C)c4ccc1c2c43 ; Cc1sccc1N(c1ccc(F)cc1)c1cc(N(c2ccc(F)cc2)c2ccsc2C)c2ccc3c(N(c4ccc(F)cc4)c4ccsc4C)cc(N(c4ccc(F)cc4)c4ccsc4C)c4ccc1c2c43 ; Cc1cccn1N(c1ccc(F)cc1)c1cc(N(c2ccc(F)cc2)n2cccc2C)c2ccc3c(N(c4ccc(F)cc4)n4cccc4C)cc(N(c4ccc(F)cc4)n4cccc4C)c4ccc1c2c43 ; Cc1ncoc1N(c1ccc(F)cc1)c1cc(N(c2ccc(F)cc2)c2ocnc2C)c2ccc3c(N(c4ccc(F)cc4)c4ocnc4C)cc(N(c4ccc(F)cc4)c4ocnc4C)c4ccc1c2c43]

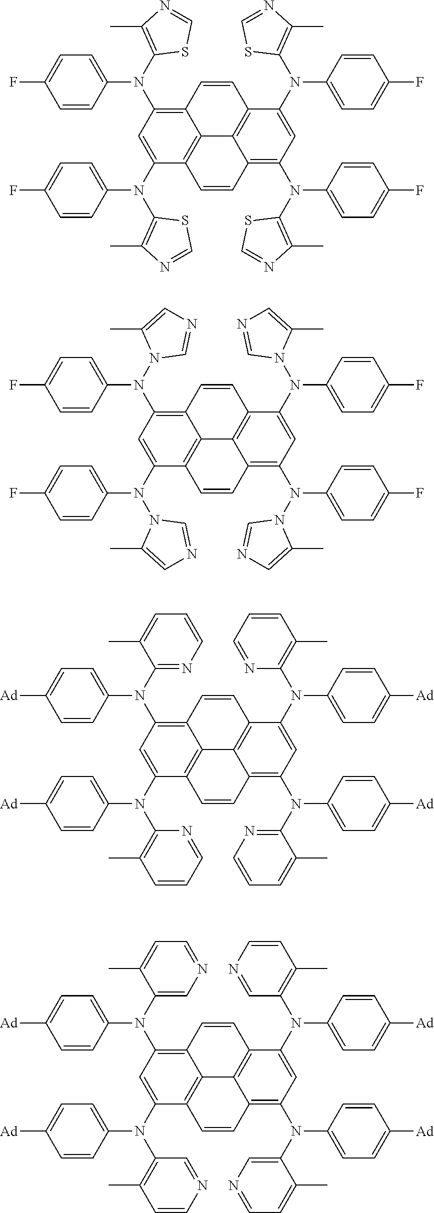

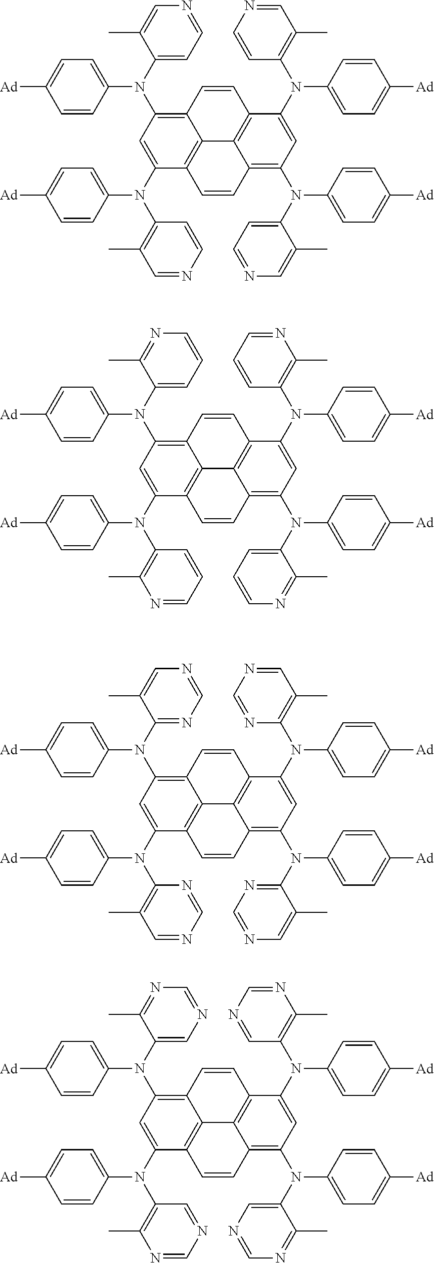

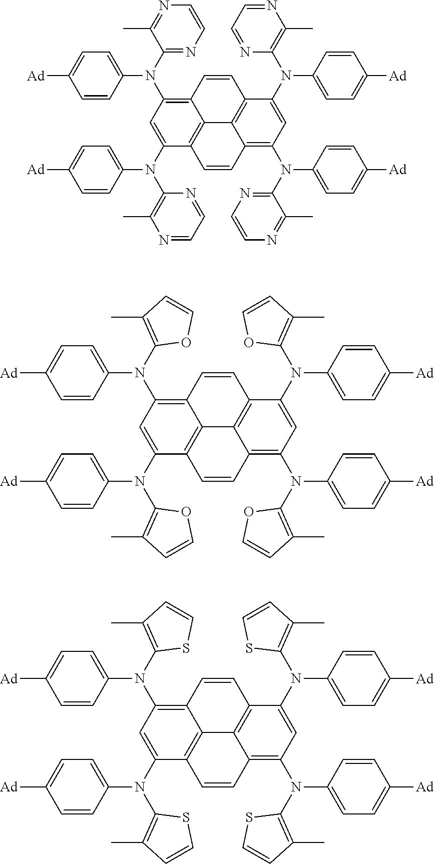

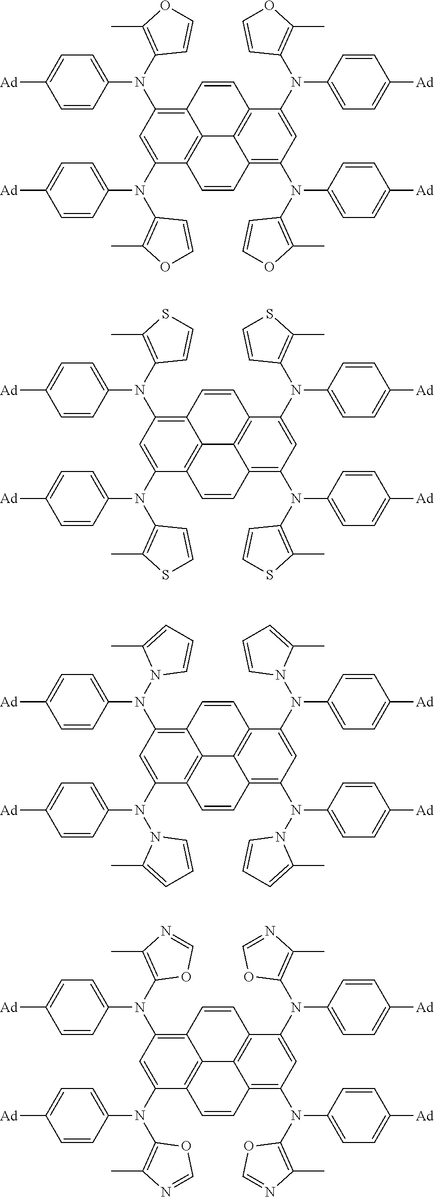

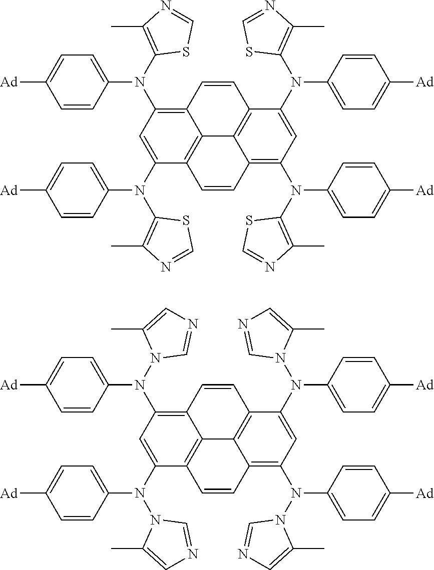

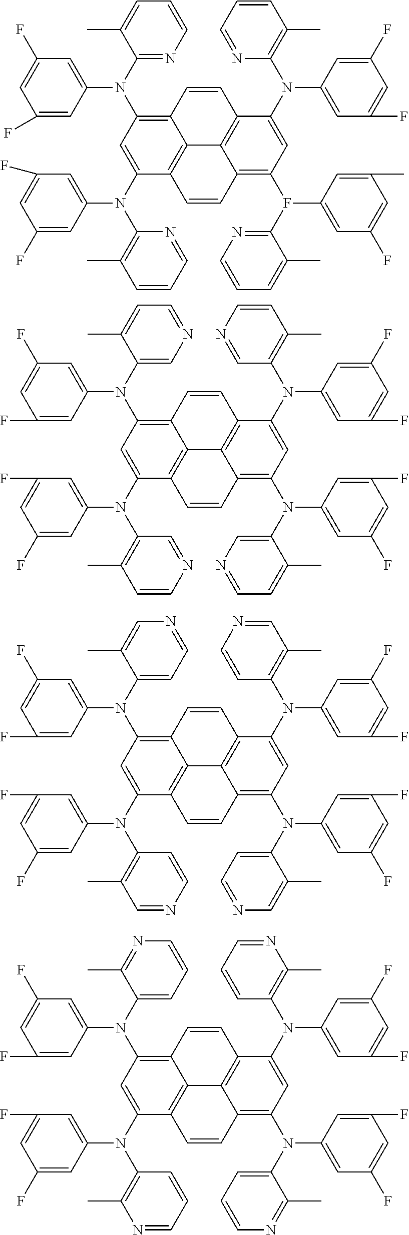

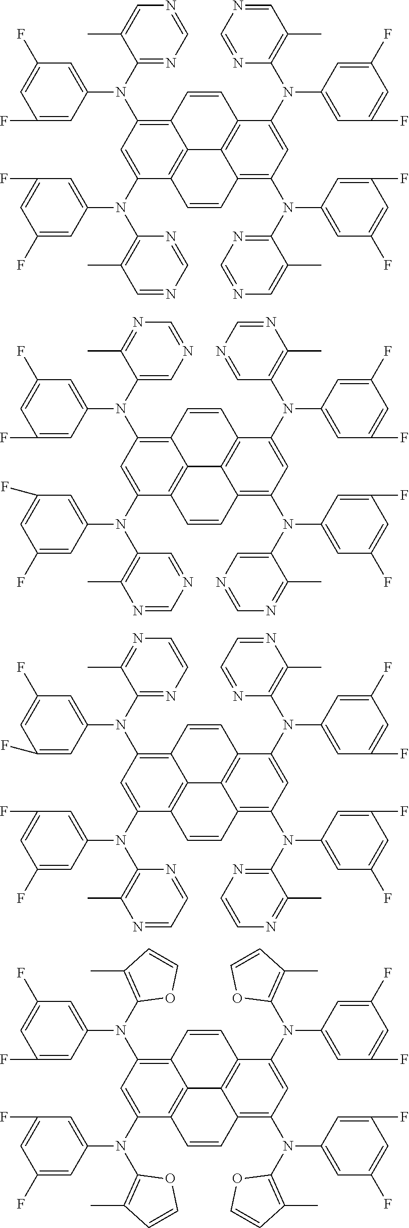

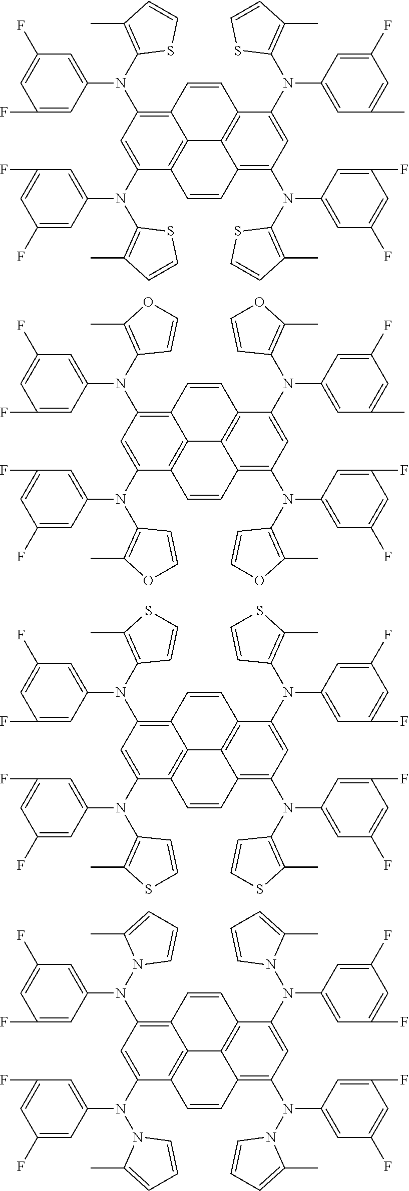

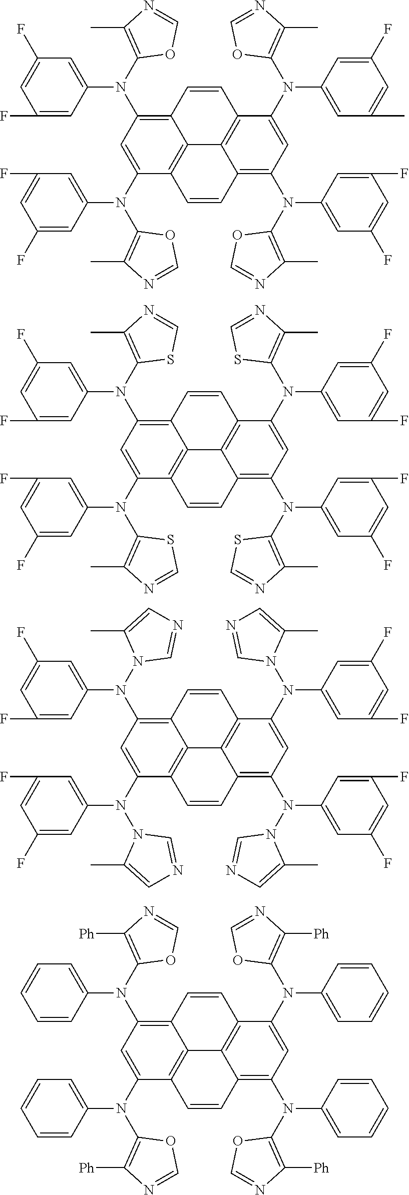

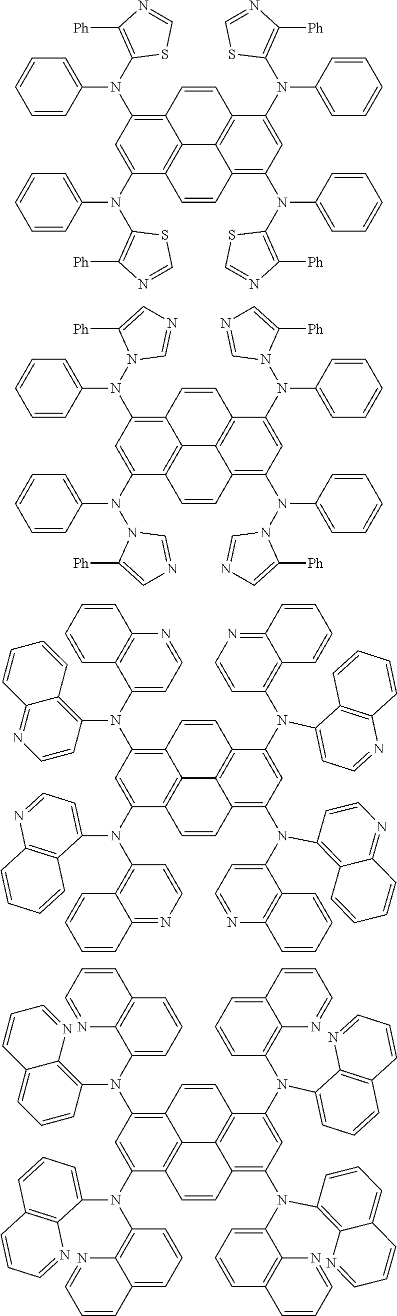

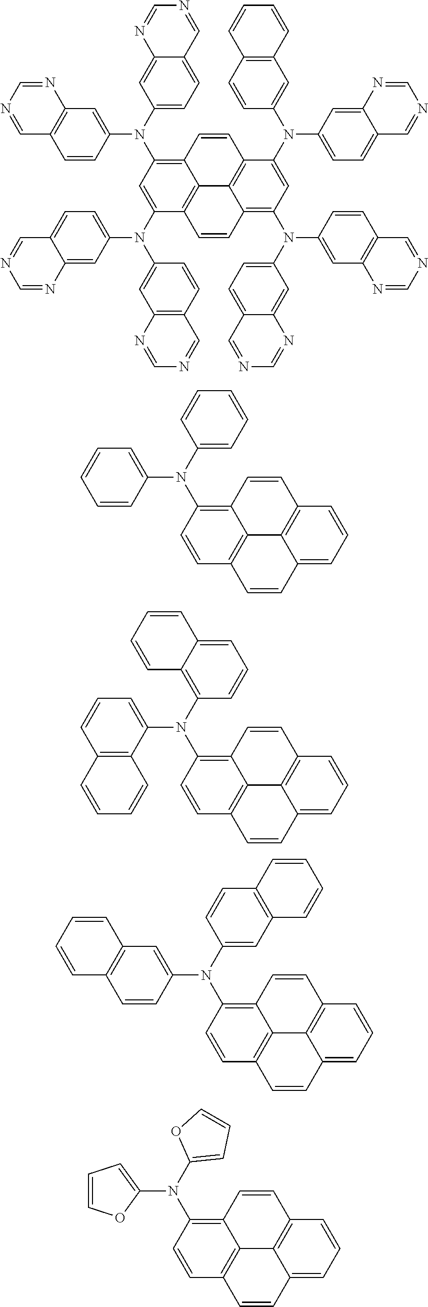

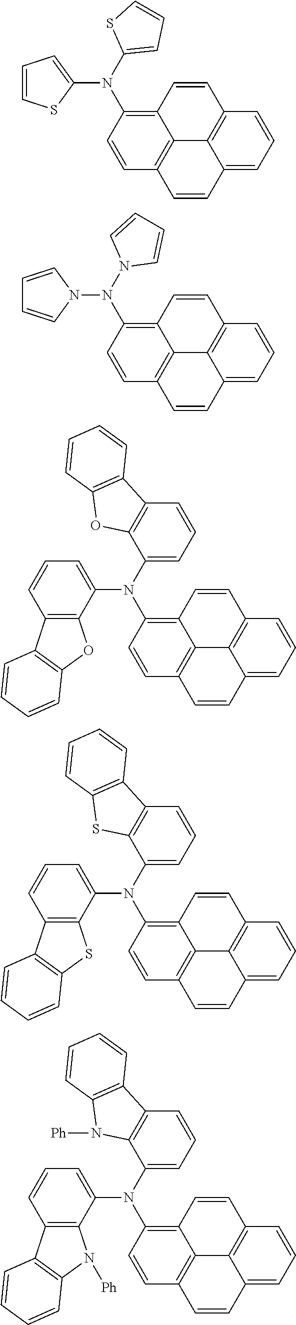

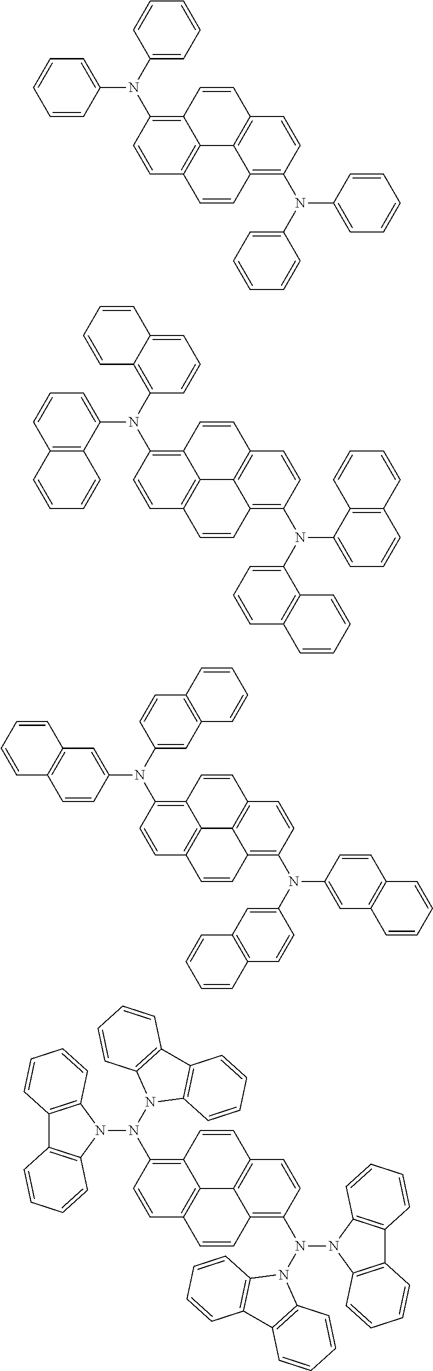 [SMILES: c1ccc(N(c2ccccc2)c2ccc3ccc4c(N(c5ccccc5)c5ccccc5)ccc5ccc2c3c54)cc1 ; c1ccc2c(N(c3cccc4ccccc34)c3ccc4ccc5c(N(c6cccc7ccccc67)c6cccc7ccccc67)ccc6ccc3c4c65)cccc2c1 ; c1ccc2cc(N(c3ccc4ccccc4c3)c3ccc4ccc5c(N(c6ccc7ccccc7c6)c6ccc7ccccc7c6)ccc6ccc3c4c65)ccc2c1 ; c1ccc2c(c1)c1ccccc1n2N(c1ccc2ccc3c(N(n4c5ccccc5c5ccccc54)n4c5ccccc5c5ccccc54)ccc4ccc1c2c43)n1c2ccccc2c2ccccc21]

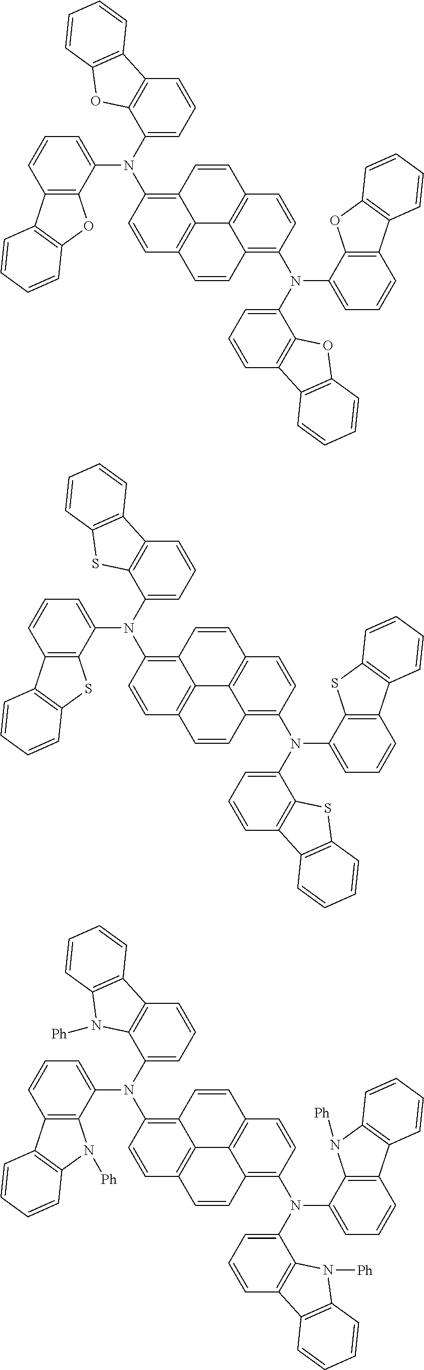 [SMILES: c1ccc2c(c1)oc1c(N(c3ccc4ccc5c(N(c6cccc7c6oc6ccccc67)c6cccc7c6oc6ccccc67)ccc6ccc3c4c65)c3cccc4c3oc3ccccc34)cccc12 ; c1ccc2c(c1)sc1c(N(c3ccc4ccc5c(N(c6cccc7c6sc6ccccc67)c6cccc7c6sc6ccccc67)ccc6ccc3c4c65)c3cccc4c3sc3ccccc34)cccc12 ; c1ccc(-n2c3ccccc3c3cccc(N(c4ccc5ccc6c(N(c7cccc8c9ccccc9n(-c9ccccc9)c78)c7cccc8c9ccccc9n(-c9ccccc9)c78)ccc7ccc4c5c76)c4cccc5c6ccccc6n(-c6ccccc6)c45)c32)cc1]

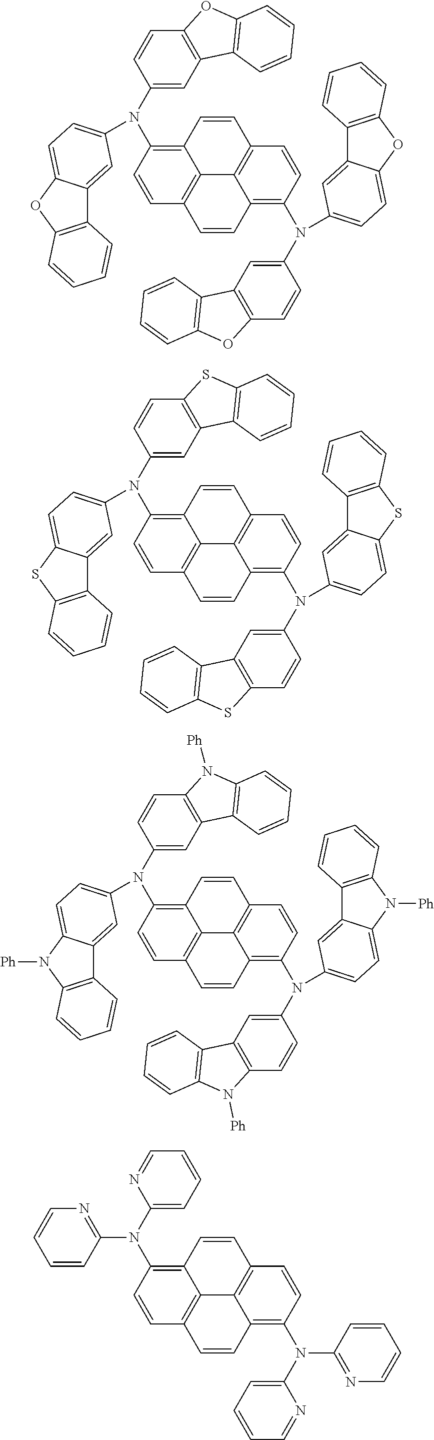

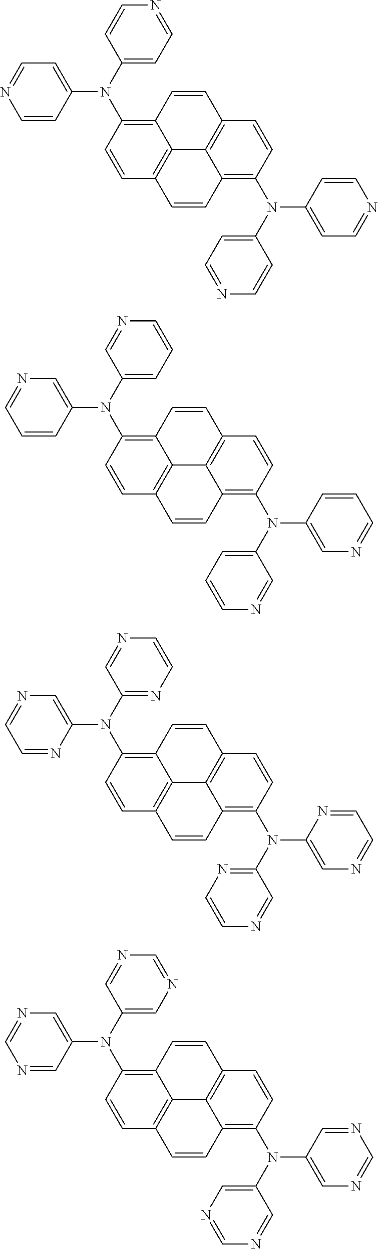 [SMILES: c1cc(N(c2ccncc2)c2ccc3ccc4c(N(c5ccncc5)c5ccncc5)ccc5ccc2c3c54)ccn1 ; c1cncc(N(c2cccnc2)c2ccc3ccc4c(N(c5cccnc5)c5cccnc5)ccc5ccc2c3c54)c1 ; c1cnc(N(c2cnccn2)c2ccc3ccc4c(N(c5cnccn5)c5cnccn5)ccc5ccc2c3c54)cn1 ; c1ncc(N(c2cncnc2)c2ccc3ccc4c(N(c5cncnc5)c5cncnc5)ccc5ccc2c3c54)cn1]

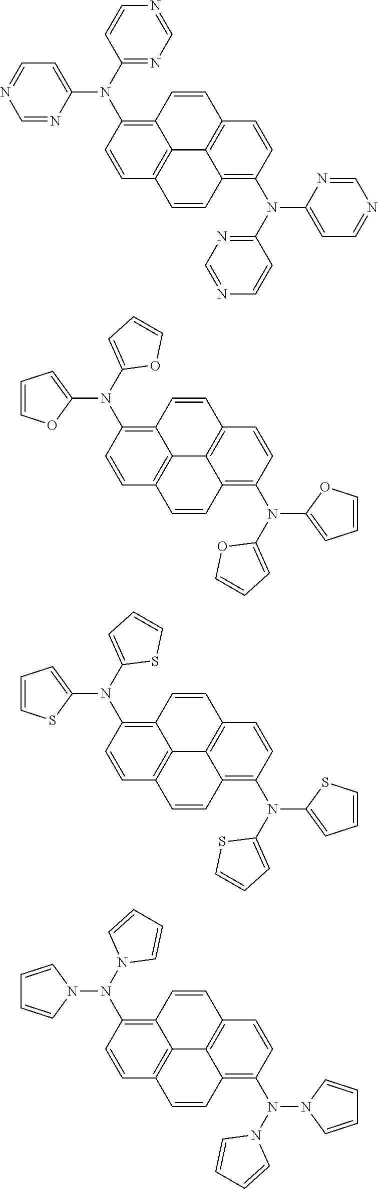 [SMILES: c1cc(N(c2ccncn2)c2ccc3ccc4c(N(c5ccncn5)c5ccncn5)ccc5ccc2c3c54)ncn1 ; c1coc(N(c2ccco2)c2ccc3ccc4c(N(c5ccco5)c5ccco5)ccc5ccc2c3c54)c1 ; c1csc(N(c2cccs2)c2ccc3ccc4c(N(c5cccs5)c5cccs5)ccc5ccc2c3c54)c1 ; c1ccn(N(c2ccc3ccc4c(N(n5cccc5)n5cccc5)ccc5ccc2c3c54)n2cccc2)c1]

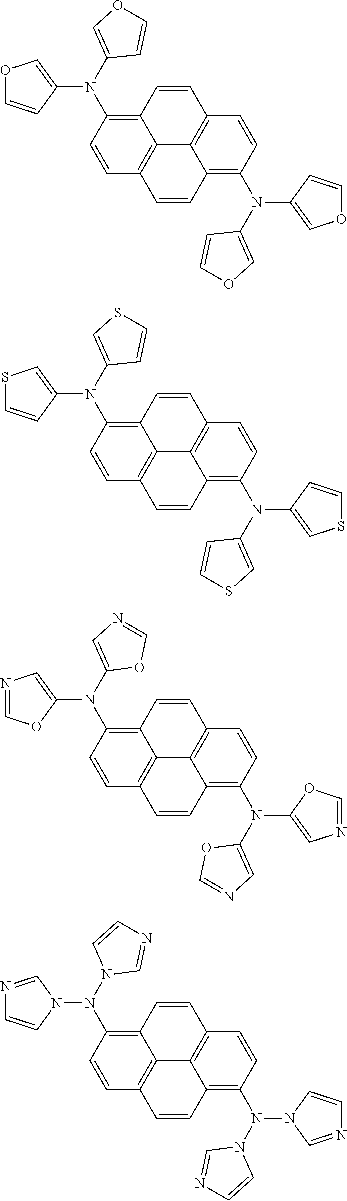 [SMILES: c1cc(N(c2ccoc2)c2ccc3ccc4c(N(c5ccoc5)c5ccoc5)ccc5ccc2c3c54)co1 ; c1cc(N(c2ccsc2)c2ccc3ccc4c(N(c5ccsc5)c5ccsc5)ccc5ccc2c3c54)cs1 ; c1ncc(N(c2cnco2)c2ccc3ccc4c(N(c5cnco5)c5cnco5)ccc5ccc2c3c54)o1 ; c1cn(N(c2ccc3ccc4c(N(n5ccnc5)n5ccnc5)ccc5ccc2c3c54)n2ccnc2)cn1]

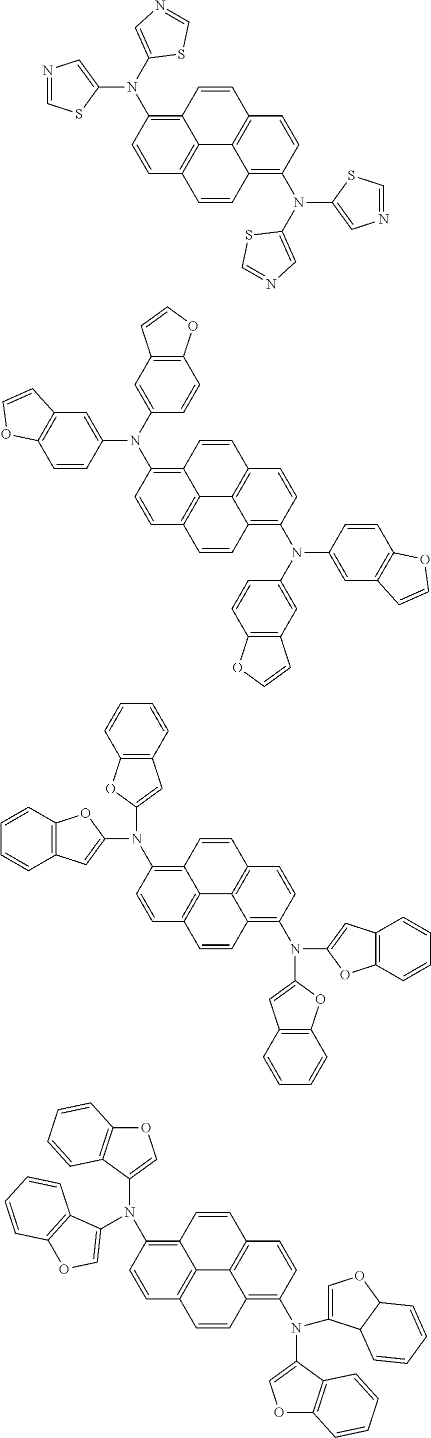 [SMILES: c1ncc(N(c2cncs2)c2ccc3ccc4c(N(c5cncs5)c5cncs5)ccc5ccc2c3c54)s1 ; c1cc2cc(N(c3ccc4occc4c3)c3ccc4ccc5c(N(c6ccc7occc7c6)c6ccc7occc7c6)ccc6ccc3c4c65)ccc2o1 ; c1ccc2oc(N(c3cc4ccccc4o3)c3ccc4ccc5c(N(c6cc7ccccc7o6)c6cc7ccccc7o6)ccc6ccc3c4c65)cc2c1 ; C1=CC2OC=C(N(c3coc4ccccc34)c3ccc4ccc5c(N(c6coc7ccccc67)c6coc7ccccc67)ccc6ccc3c4c65)C2C=C1]

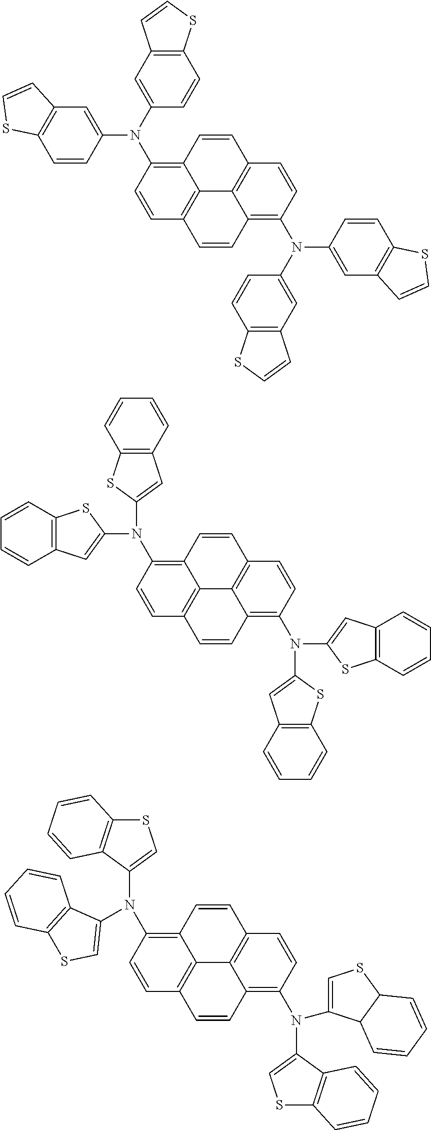 [SMILES: c1cc2cc(N(c3ccc4sccc4c3)c3ccc4ccc5c(N(c6ccc7sccc7c6)c6ccc7sccc7c6)ccc6ccc3c4c65)ccc2s1 ; c1ccc2sc(N(c3cc4ccccc4s3)c3ccc4ccc5c(N(c6cc7ccccc7s6)c6cc7ccccc7s6)ccc6ccc3c4c65)cc2c1 ; C1=CC2SC=C(N(c3csc4ccccc34)c3ccc4ccc5c(N(c6csc7ccccc67)c6csc7ccccc67)ccc6ccc3c4c65)C2C=C1]

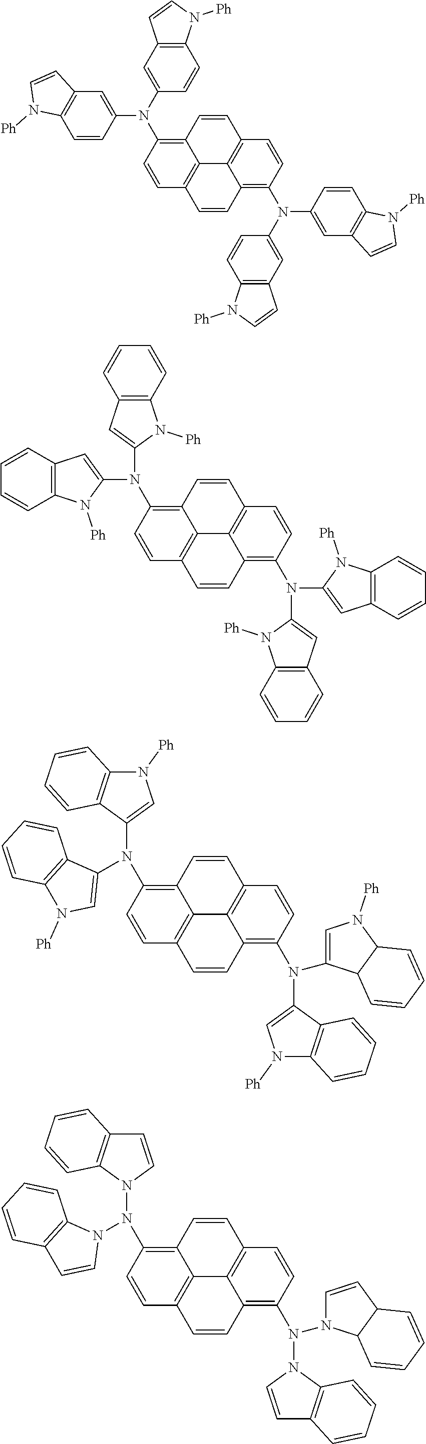 [SMILES: c1ccc(-n2ccc3cc(N(c4ccc5c(ccn5-c5ccccc5)c4)c4ccc5ccc6c(N(c7ccc8c(ccn8-c8ccccc8)c7)c7ccc8c(ccn8-c8ccccc8)c7)ccc7ccc4c5c76)ccc32)cc1 ; c1ccc(-n2c(N(c3ccc4ccc5c(N(c6cc7ccccc7n6-c6ccccc6)c6cc7ccccc7n6-c6ccccc6)ccc6ccc3c4c65)c3cc4ccccc4n3-c3ccccc3)cc3ccccc32)cc1 ; C1=CC2C(N(c3ccc4ccc5c(N(c6cn(-c7ccccc7)c7ccccc67)c6cn(-c7ccccc7)c7ccccc67)ccc6ccc3c4c65)c3cn(-c4ccccc4)c4ccccc34)=CN(c3ccccc3)C2C=C1 ; C1=CC2C=CN(N(c3ccc4ccc5c(N(n6ccc7ccccc76)n6ccc7ccccc76)ccc6ccc3c4c65)n3ccc4ccccc43)C2C=C1]

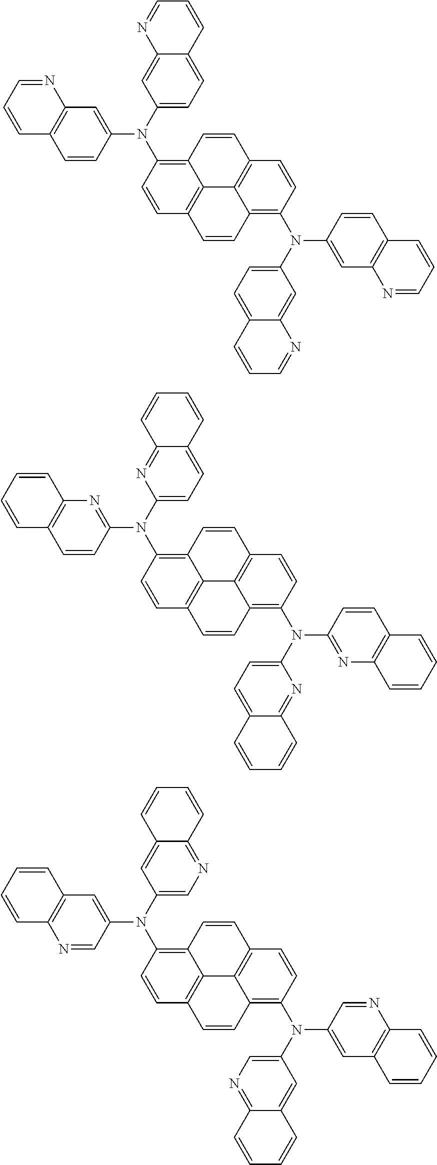 [SMILES: c1cnc2cc(N(c3ccc4cccnc4c3)c3ccc4ccc5c(N(c6ccc7cccnc7c6)c6ccc7cccnc7c6)ccc6ccc3c4c65)ccc2c1 ; c1ccc2nc(N(c3ccc4ccccc4n3)c3ccc4ccc5c(N(c6ccc7ccccc7n6)c6ccc7ccccc7n6)ccc6ccc3c4c65)ccc2c1 ; c1ccc2ncc(N(c3cnc4ccccc4c3)c3ccc4ccc5c(N(c6cnc7ccccc7c6)c6cnc7ccccc7c6)ccc6ccc3c4c65)cc2c1]

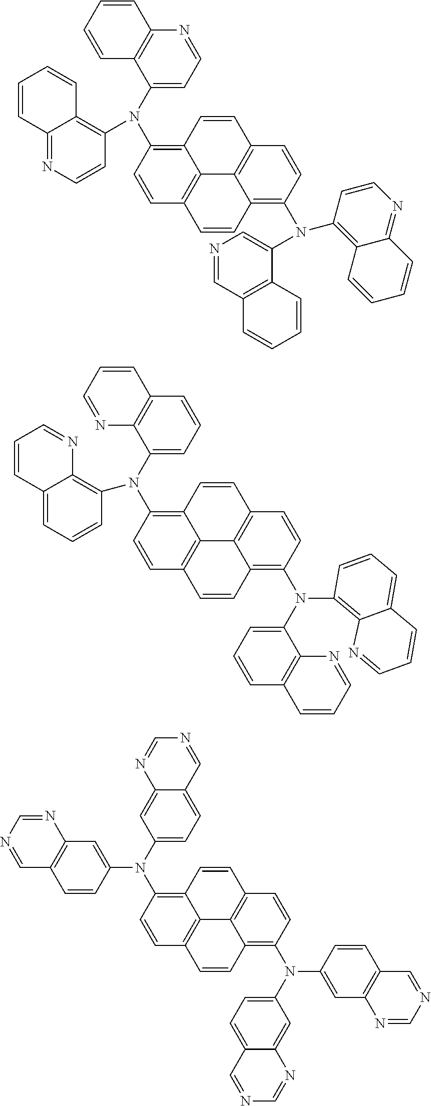 [SMILES: c1ccc2c(N(c3ccnc4ccccc34)c3ccc4ccc5c(N(c6ccnc7ccccc67)c6ccnc7ccccc67)ccc6ccc3c4c65)cncc2c1 ; c1cnc2c(N(c3ccc4ccc5c(N(c6cccc7cccnc67)c6cccc7cccnc67)ccc6ccc3c4c65)c3cccc4cccnc34)cccc2c1 ; c1ncc2ccc(N(c3ccc4cncnc4c3)c3ccc4ccc5c(N(c6ccc7cncnc7c6)c6ccc7cncnc7c6)ccc6ccc3c4c65)cc2n1]

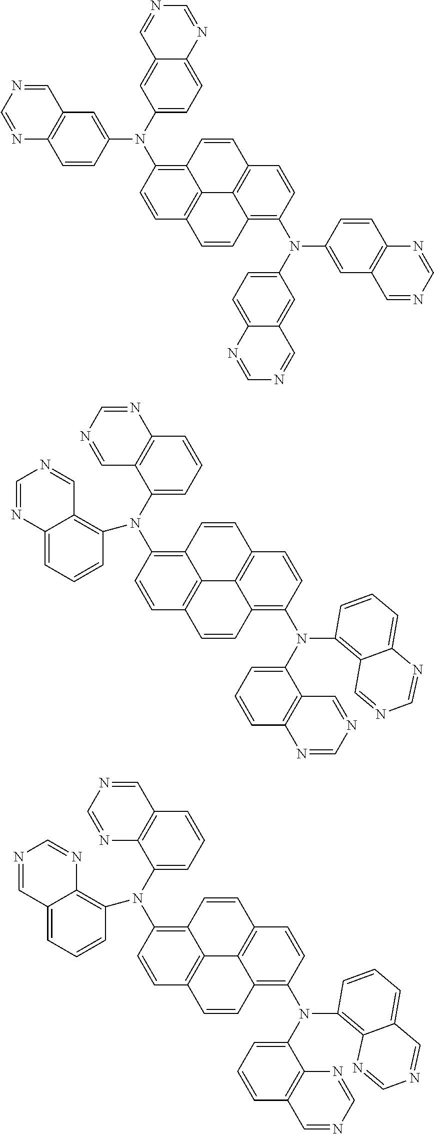 [SMILES: c1ncc2cc(N(c3ccc4ncncc4c3)c3ccc4ccc5c(N(c6ccc7ncncc7c6)c6ccc7ncncc7c6)ccc6ccc3c4c65)ccc2n1 ; c1cc(N(c2cccc3ncncc23)c2ccc3ccc4c(N(c5cccc6ncncc56)c5cccc6ncncc56)ccc5ccc2c3c54)c2cncnc2c1 ; c1cc(N(c2ccc3ccc4c(N(c5cccc6cncnc56)c5cccc6cncnc56)ccc5ccc2c3c54)c2cccc3cncnc23)c2ncncc2c1]

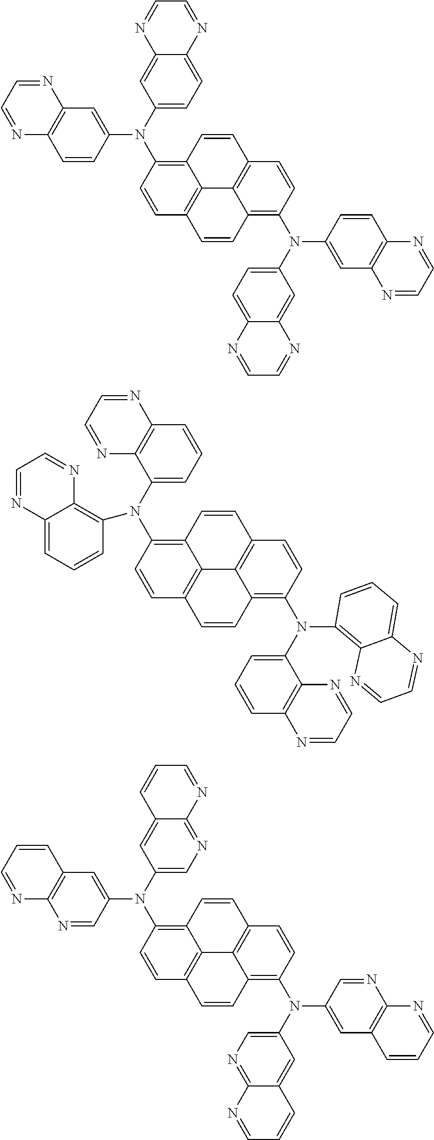 [SMILES: c1cnc2cc(N(c3ccc4nccnc4c3)c3ccc4ccc5c(N(c6ccc7nccnc7c6)c6ccc7nccnc7c6)ccc6ccc3c4c65)ccc2n1 ; c1cc(N(c2ccc3ccc4c(N(c5cccc6nccnc56)c5cccc6nccnc56)ccc5ccc2c3c54)c2cccc3nccnc23)c2nccnc2c1 ; c1cnc2ncc(N(c3cnc4ncccc4c3)c3ccc4ccc5c(N(c6cnc7ncccc7c6)c6cnc7ncccc7c6)ccc6ccc3c4c65)cc2c1]

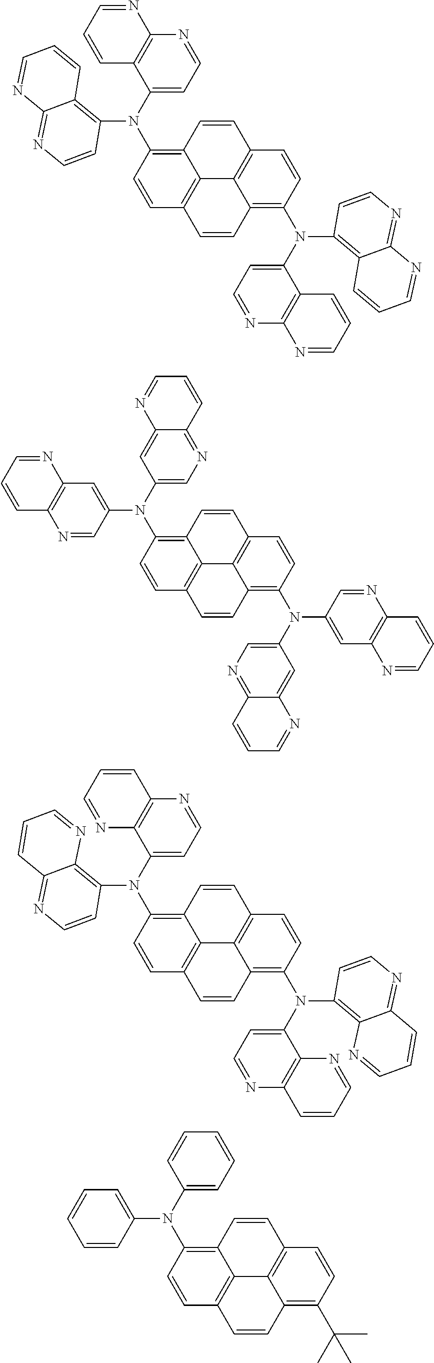 [SMILES: c1cnc2nccc(N(c3ccnc4ncccc34)c3ccc4ccc5c(N(c6ccnc7ncccc67)c6ccnc7ncccc67)ccc6ccc3c4c65)c2c1 ; c1cnc2cc(N(c3cnc4cccnc4c3)c3ccc4ccc5c(N(c6cnc7cccnc7c6)c6cnc7cccnc7c6)ccc6ccc3c4c65)cnc2c1 ; c1cnc2c(N(c3ccc4ccc5c(N(c6ccnc7cccnc67)c6ccnc7cccnc67)ccc6ccc3c4c65)c3ccnc4cccnc34)ccnc2c1 ; CC(C)(C)c1ccc2ccc3c(N(c4ccccc4)c4ccccc4)ccc4ccc1c2c43]

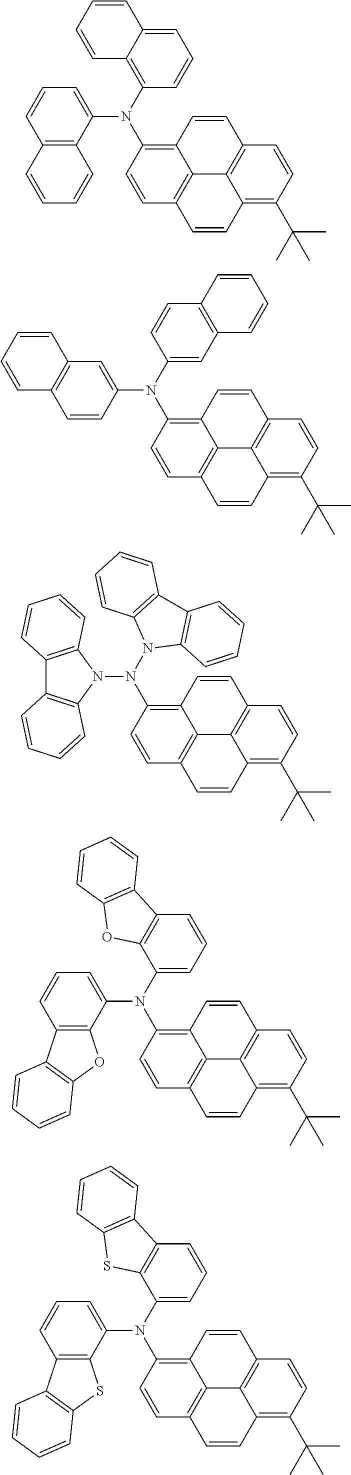

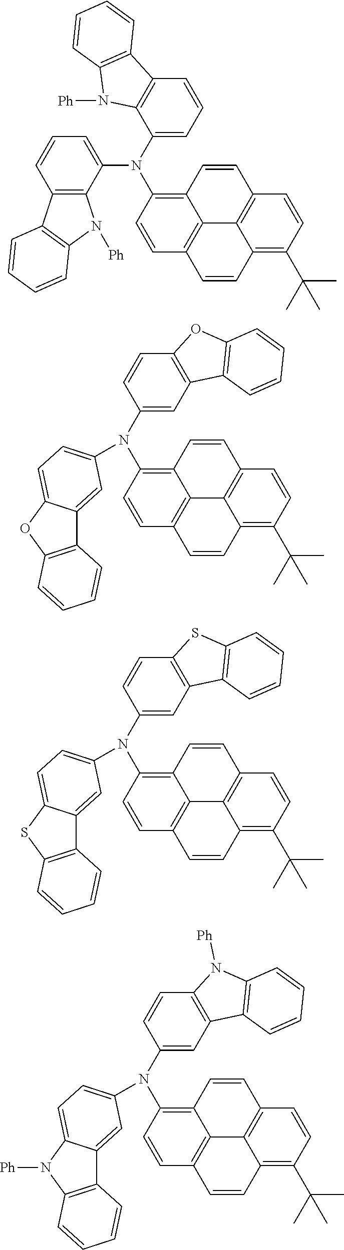

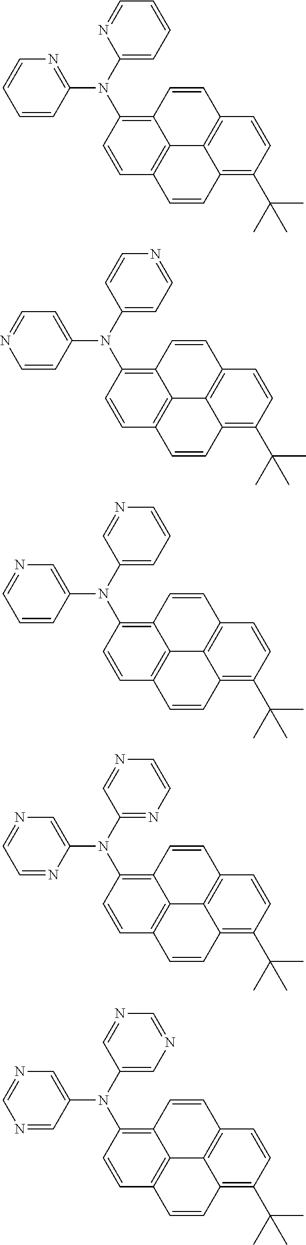 [SMILES: CC(C)(C)c1ccc2ccc3c(N(c4ccccn4)c4ccccn4)ccc4ccc1c2c43 ; CC(C)(C)c1ccc2ccc3c(N(c4ccncc4)c4ccncc4)ccc4ccc1c2c43 ; CC(C)(C)c1ccc2ccc3c(N(c4cccnc4)c4cccnc4)ccc4ccc1c2c43 ; CC(C)(C)c1ccc2ccc3c(N(c4cnccn4)c4cnccn4)ccc4ccc1c2c43 ; CC(C)(C)c1ccc2ccc3c(N(c4cncnc4)c4cncnc4)ccc4ccc1c2c43]

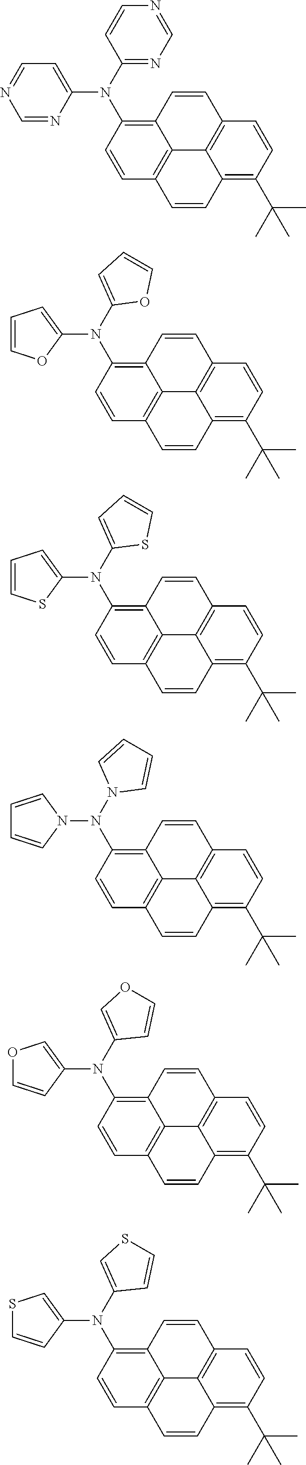 [SMILES: CC(C)(C)c1ccc2ccc3c(N(c4ccncn4)c4ccncn4)ccc4ccc1c2c43 ; CC(C)(C)c1ccc2ccc3c(N(c4ccco4)c4ccco4)ccc4ccc1c2c43 ; CC(C)(C)c1ccc2ccc3c(N(c4cccs4)c4cccs4)ccc4ccc1c2c43 ; CC(C)(C)c1ccc2ccc3c(N(n4cccc4)n4cccc4)ccc4ccc1c2c43 ; CC(C)(C)c1ccc2ccc3c(N(c4ccoc4)c4ccoc4)ccc4ccc1c2c43 ; CC(C)(C)c1ccc2ccc3c(N(c4ccsc4)c4ccsc4)ccc4ccc1c2c43]

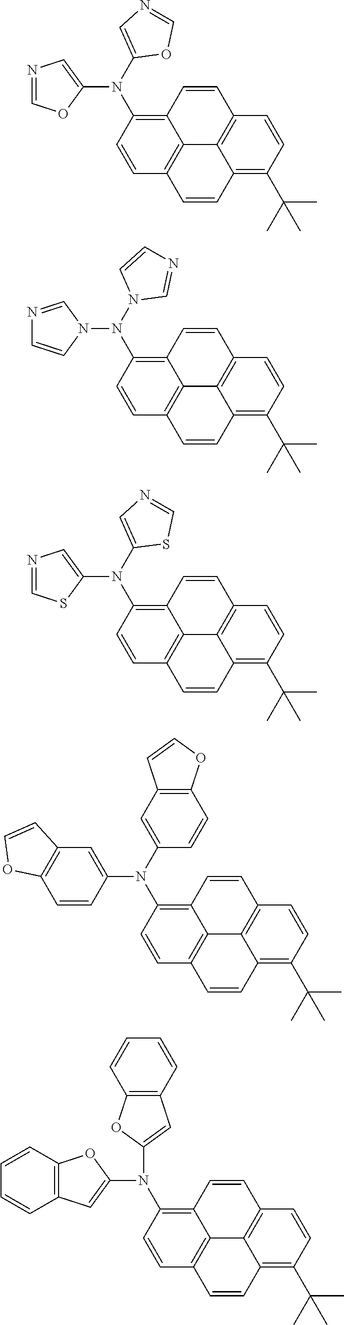 [SMILES: CC(C)(C)c1ccc2ccc3c(N(c4cnco4)c4cnco4)ccc4ccc1c2c43 ; CC(C)(C)c1ccc2ccc3c(N(n4ccnc4)n4ccnc4)ccc4ccc1c2c43 ; CC(C)(C)c1ccc2ccc3c(N(c4cncs4)c4cncs4)ccc4ccc1c2c43 ; CC(C)(C)c1ccc2ccc3c(N(c4ccc5occc5c4)c4ccc5occc5c4)ccc4ccc1c2c43 ; CC(C)(C)c1ccc2ccc3c(N(c4cc5ccccc5o4)c4cc5ccccc5o4)ccc4ccc1c2c43]

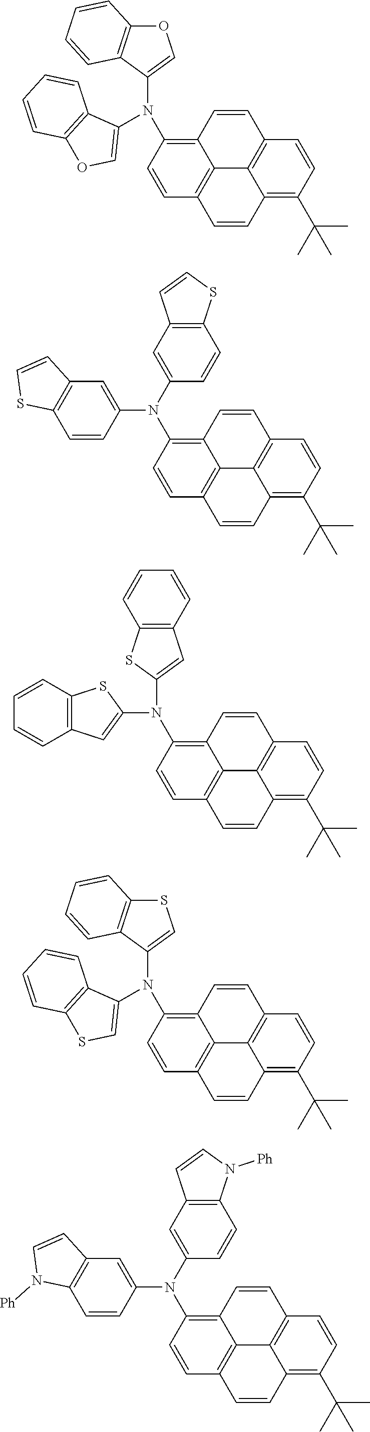

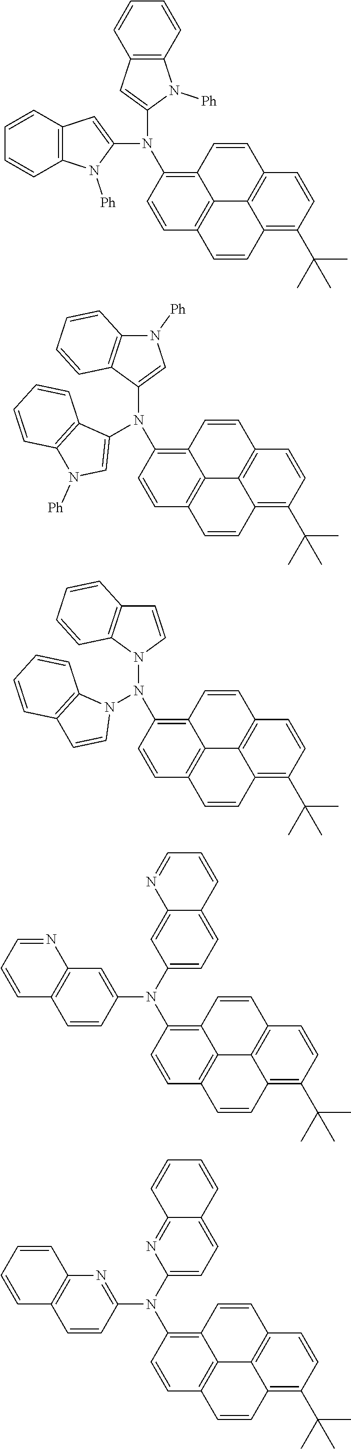

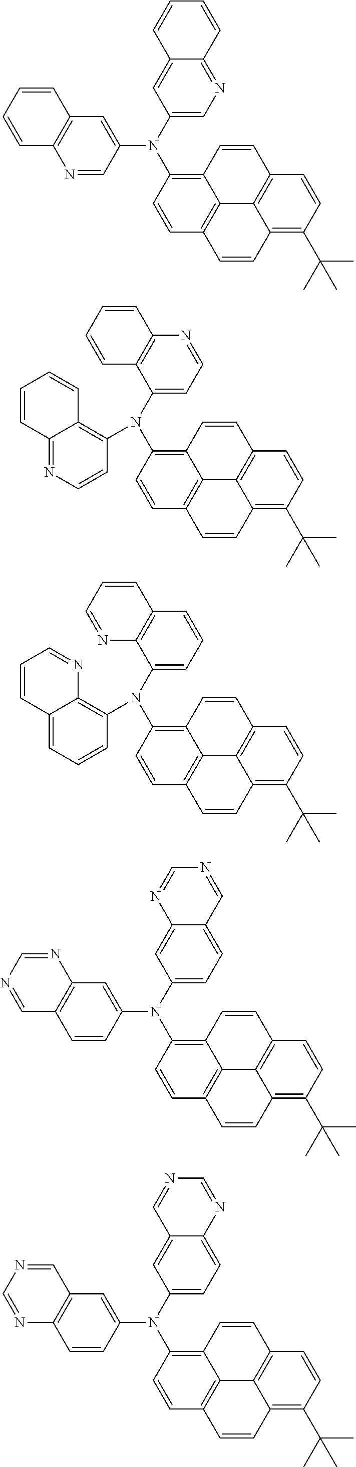 [SMILES: CC(C)(C)c1ccc2ccc3c(N(c4cnc5ccccc5c4)c4cnc5ccccc5c4)ccc4ccc1c2c43 ; CC(C)(C)c1ccc2ccc3c(N(c4ccnc5ccccc45)c4ccnc5ccccc45)ccc4ccc1c2c43 ; CC(C)(C)c1ccc2ccc3c(N(c4cccc5cccnc45)c4cccc5cccnc45)ccc4ccc1c2c43 ; CC(C)(C)c1ccc2ccc3c(N(c4ccc5cncnc5c4)c4ccc5cncnc5c4)ccc4ccc1c2c43 ; CC(C)(C)c1ccc2ccc3c(N(c4ccc5ncncc5c4)c4ccc5ncncc5c4)ccc4ccc1c2c43]

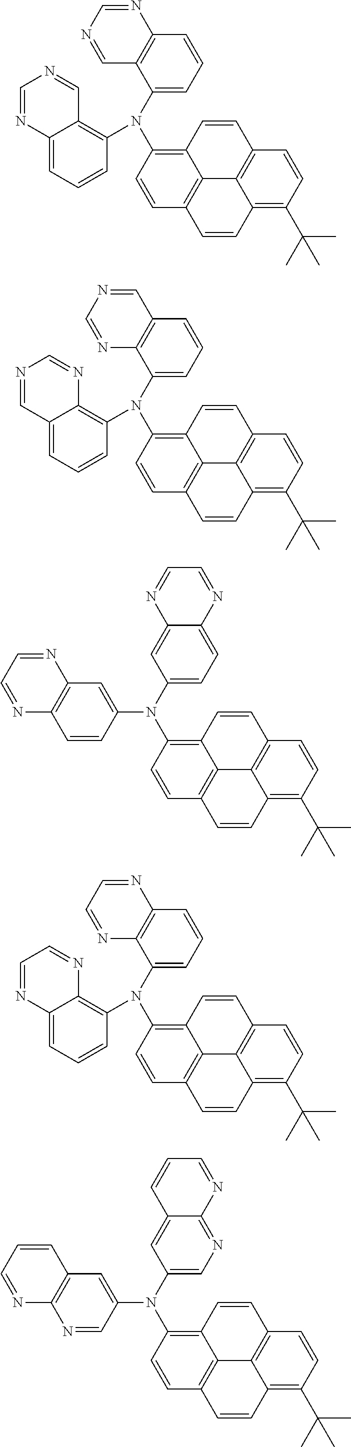 [SMILES: CC(C)(C)c1ccc2ccc3c(N(c4cccc5ncncc45)c4cccc5ncncc45)ccc4ccc1c2c43 ; CC(C)(C)c1ccc2ccc3c(N(c4cccc5cncnc45)c4cccc5cncnc45)ccc4ccc1c2c43 ; CC(C)(C)c1ccc2ccc3c(N(c4ccc5nccnc5c4)c4ccc5nccnc5c4)ccc4ccc1c2c43 ; CC(C)(C)c1ccc2ccc3c(N(c4cccc5nccnc45)c4cccc5nccnc45)ccc4ccc1c2c43 ; CC(C)(C)c1ccc2ccc3c(N(c4cnc5ncccc5c4)c4cnc5ncccc5c4)ccc4ccc1c2c43]

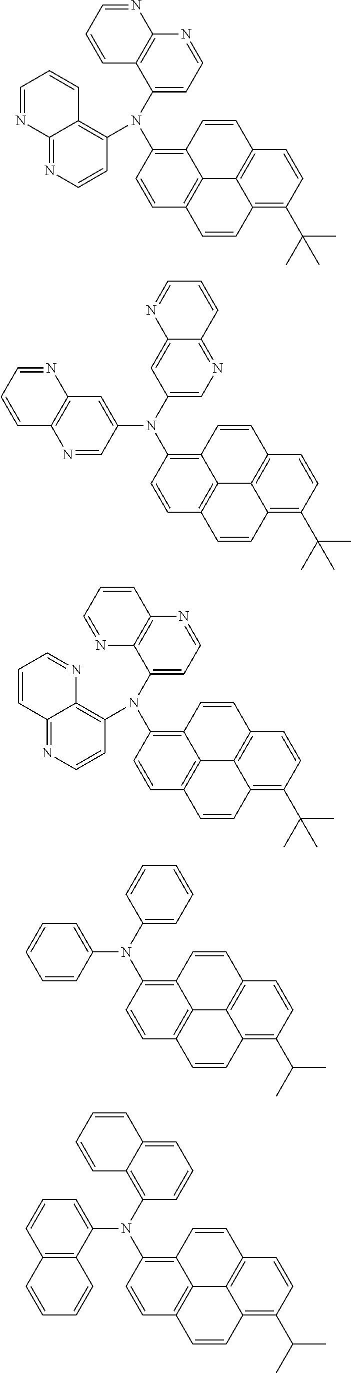

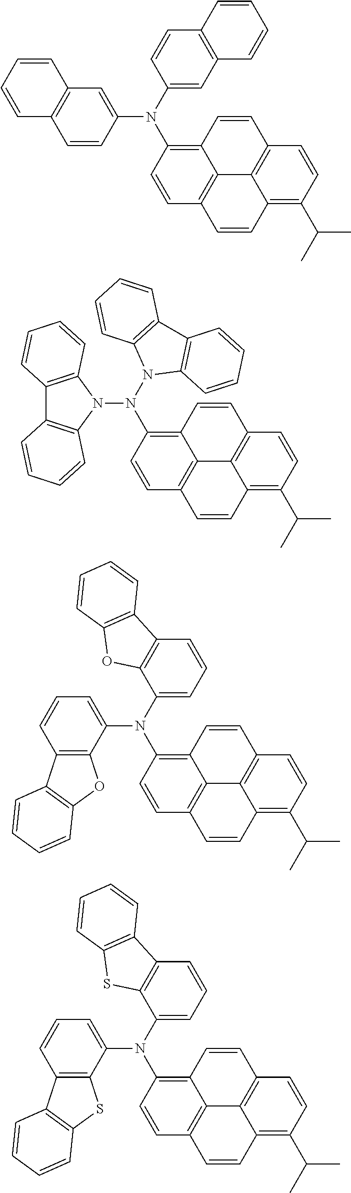 [SMILES: CC(C)c1ccc2ccc3c(N(c4ccc5ccccc5c4)c4ccc5ccccc5c4)ccc4ccc1c2c43 ; CC(C)c1ccc2ccc3c(N(n4c5ccccc5c5ccccc54)n4c5ccccc5c5ccccc54)ccc4ccc1c2c43 ; CC(C)c1ccc2ccc3c(N(c4cccc5c4oc4ccccc45)c4cccc5c4oc4ccccc45)ccc4ccc1c2c43 ; CC(C)c1ccc2ccc3c(N(c4cccc5c4sc4ccccc45)c4cccc5c4sc4ccccc45)ccc4ccc1c2c43]

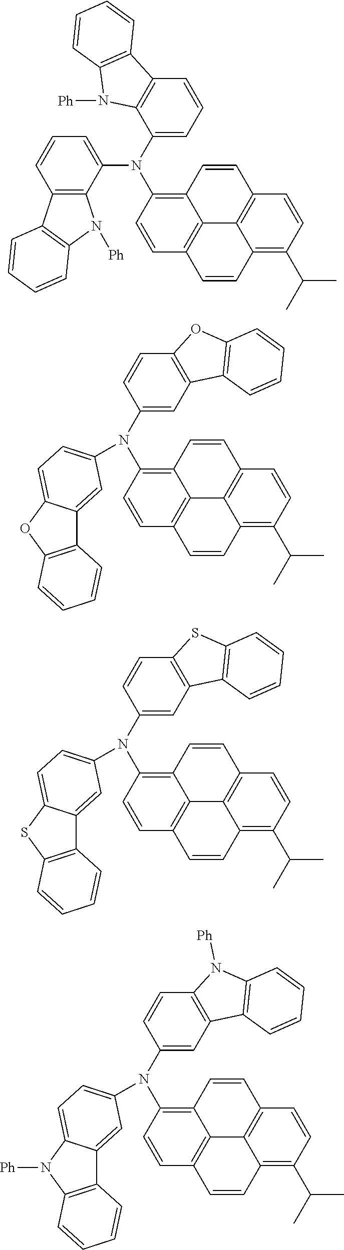

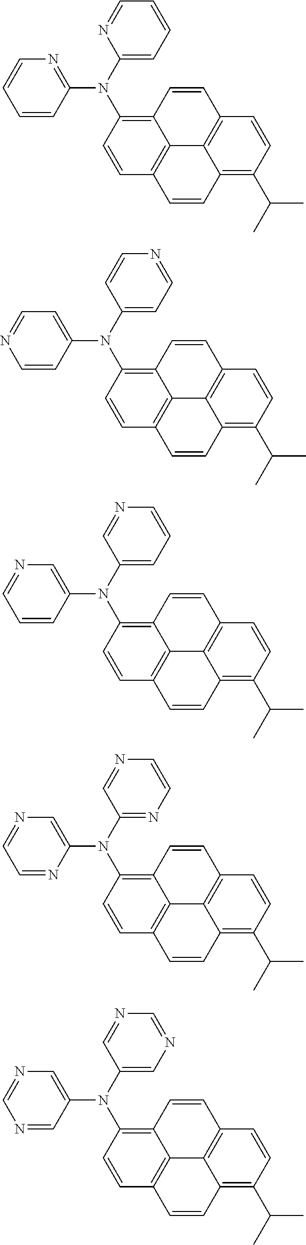 [SMILES: CC(C)c1ccc2ccc3c(N(c4ccccn4)c4ccccn4)ccc4ccc1c2c43 ; CC(C)c1ccc2ccc3c(N(c4ccncc4)c4ccncc4)ccc4ccc1c2c43 ; CC(C)c1ccc2ccc3c(N(c4cccnc4)c4cccnc4)ccc4ccc1c2c43 ; CC(C)c1ccc2ccc3c(N(c4cnccn4)c4cnccn4)ccc4ccc1c2c43 ; CC(C)c1ccc2ccc3c(N(c4cncnc4)c4cncnc4)ccc4ccc1c2c43]

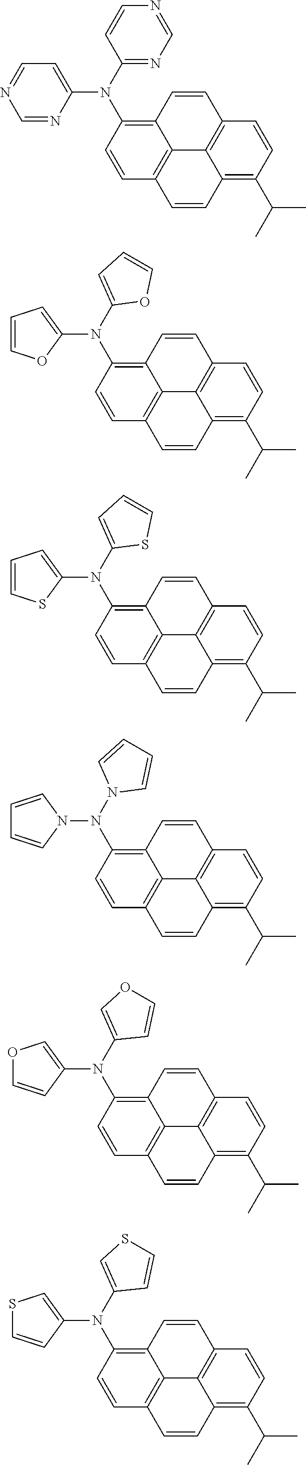 [SMILES: CC(C)c1ccc2ccc3c(N(c4ccncn4)c4ccncn4)ccc4ccc1c2c43 ; CC(C)c1ccc2ccc3c(N(c4ccco4)c4ccco4)ccc4ccc1c2c43 ; CC(C)c1ccc2ccc3c(N(c4cccs4)c4cccs4)ccc4ccc1c2c43 ; CC(C)c1ccc2ccc3c(N(n4cccc4)n4cccc4)ccc4ccc1c2c43 ; CC(C)c1ccc2ccc3c(N(c4ccoc4)c4ccoc4)ccc4ccc1c2c43 ; CC(C)c1ccc2ccc3c(N(c4ccsc4)c4ccsc4)ccc4ccc1c2c43]

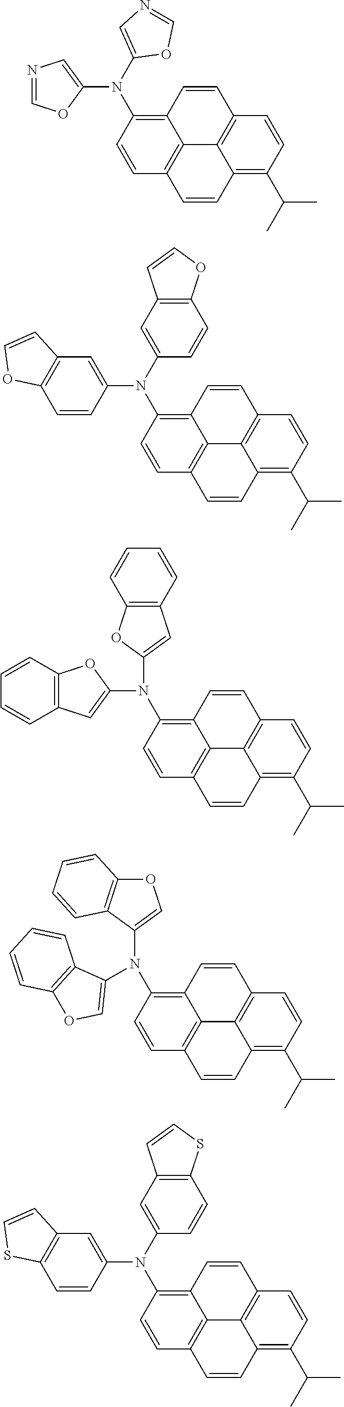

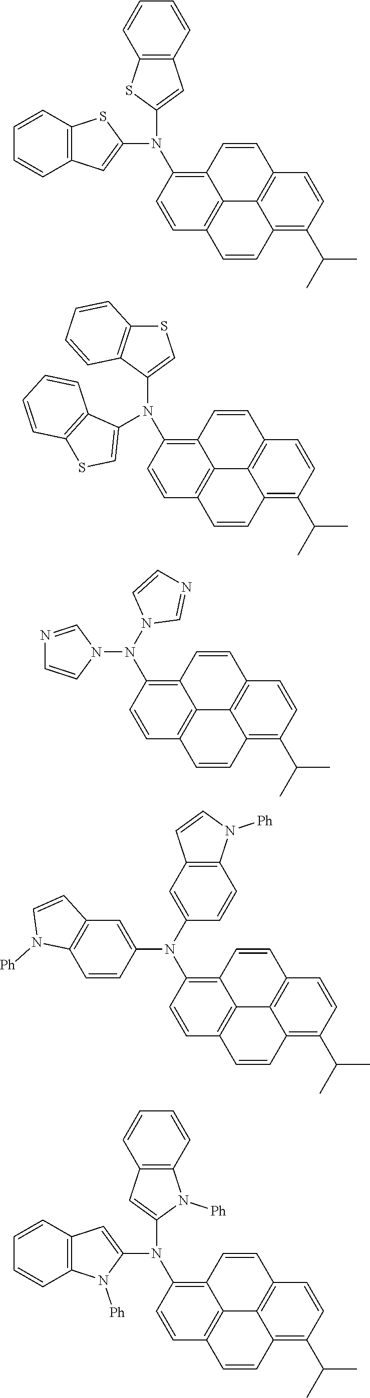

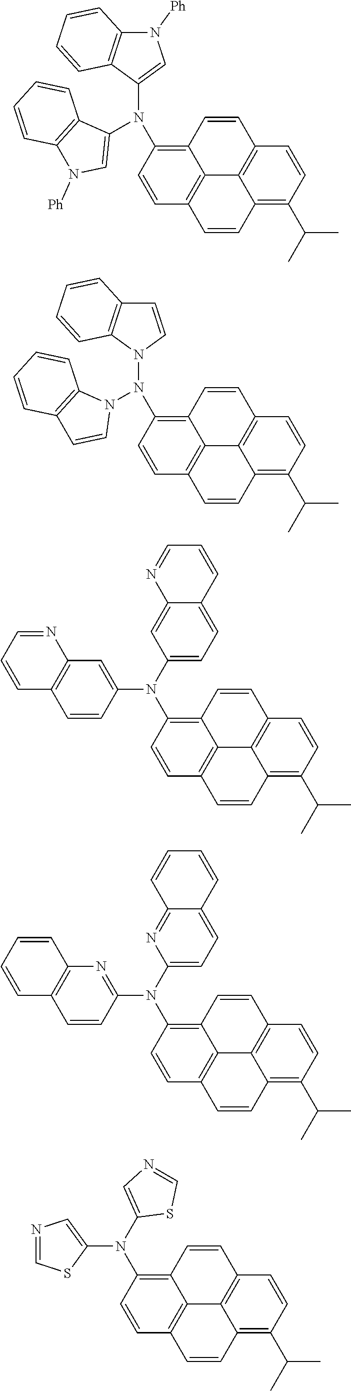

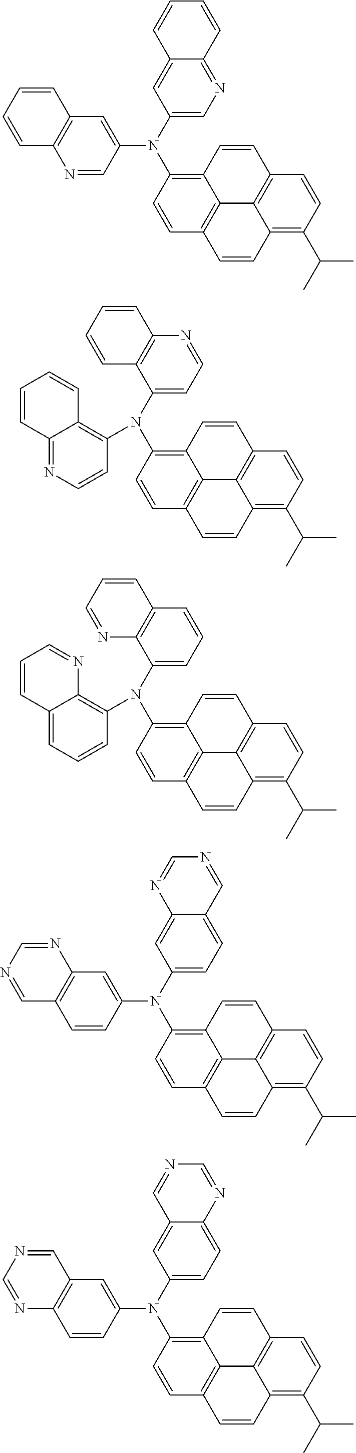 [SMILES: CC(C)c1ccc2ccc3c(N(c4cnc5ccccc5c4)c4cnc5ccccc5c4)ccc4ccc1c2c43 ; CC(C)c1ccc2ccc3c(N(c4ccnc5ccccc45)c4ccnc5ccccc45)ccc4ccc1c2c43 ; CC(C)c1ccc2ccc3c(N(c4cccc5cccnc45)c4cccc5cccnc45)ccc4ccc1c2c43 ; CC(C)c1ccc2ccc3c(N(c4ccc5cncnc5c4)c4ccc5cncnc5c4)ccc4ccc1c2c43 ; CC(C)c1ccc2ccc3c(N(c4ccc5ncncc5c4)c4ccc5ncncc5c4)ccc4ccc1c2c43]

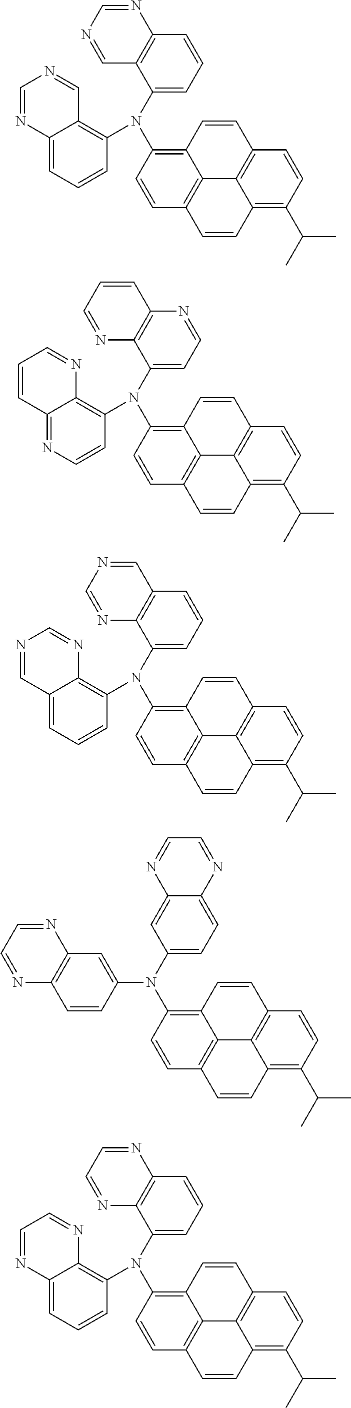

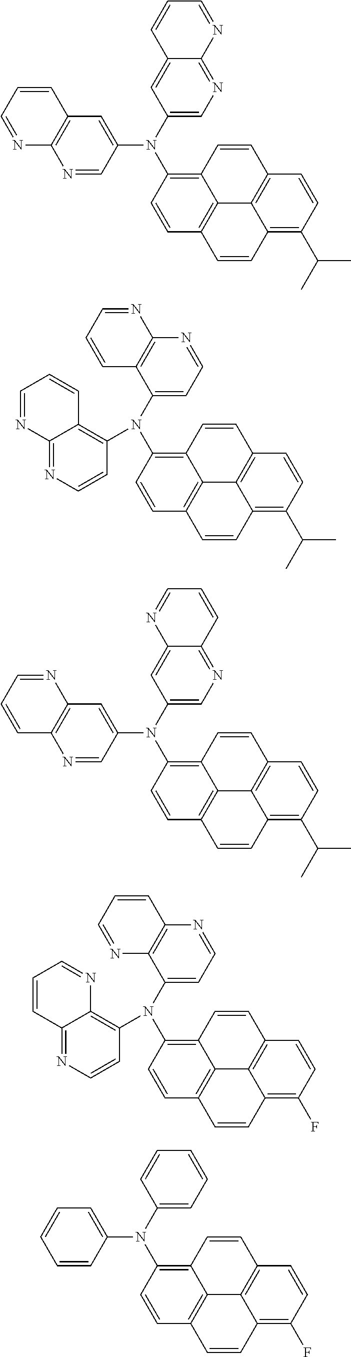

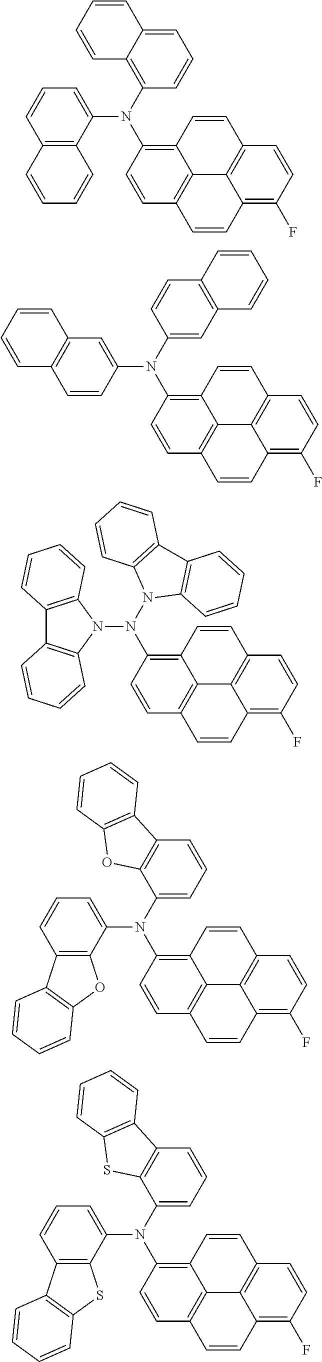

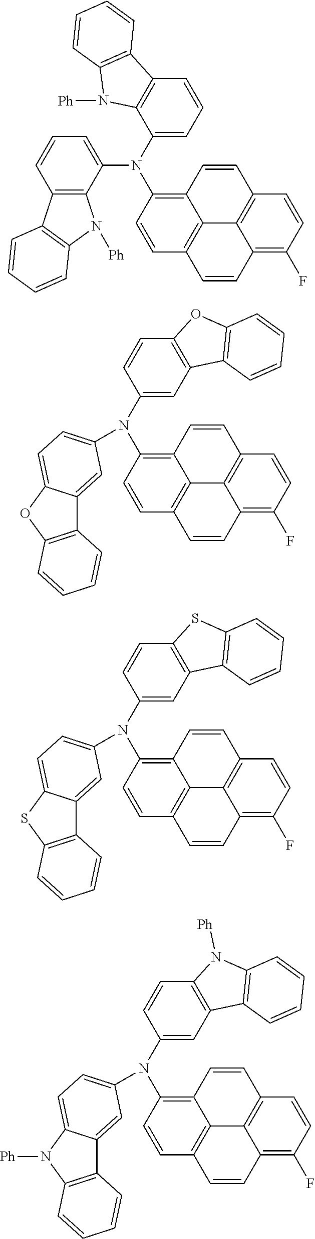

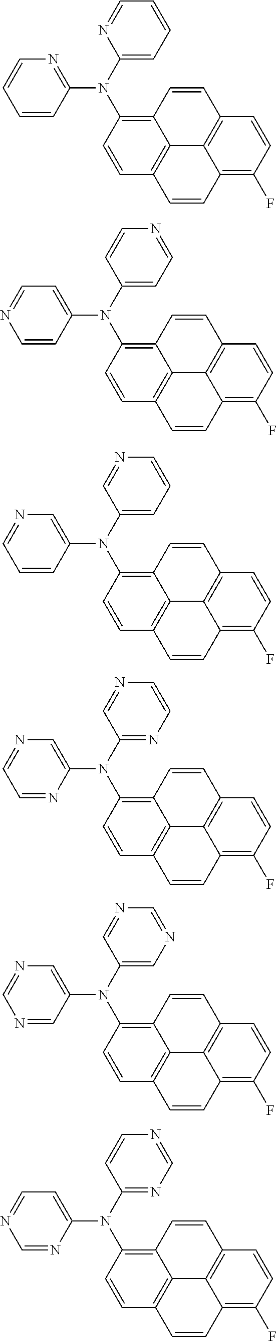

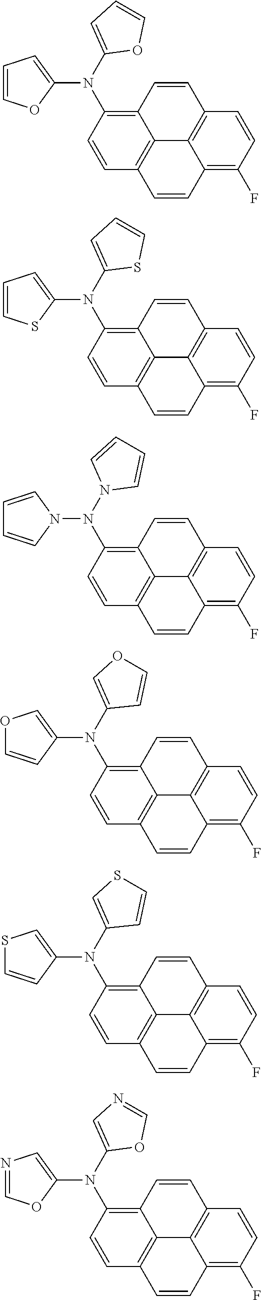

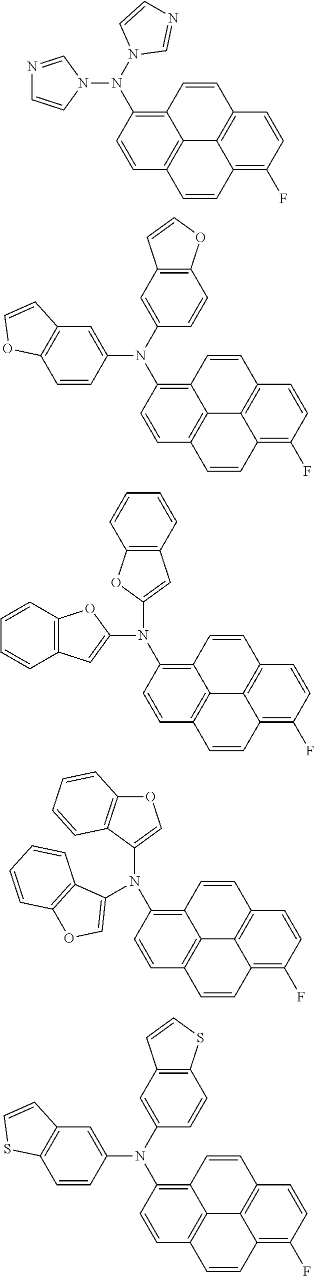

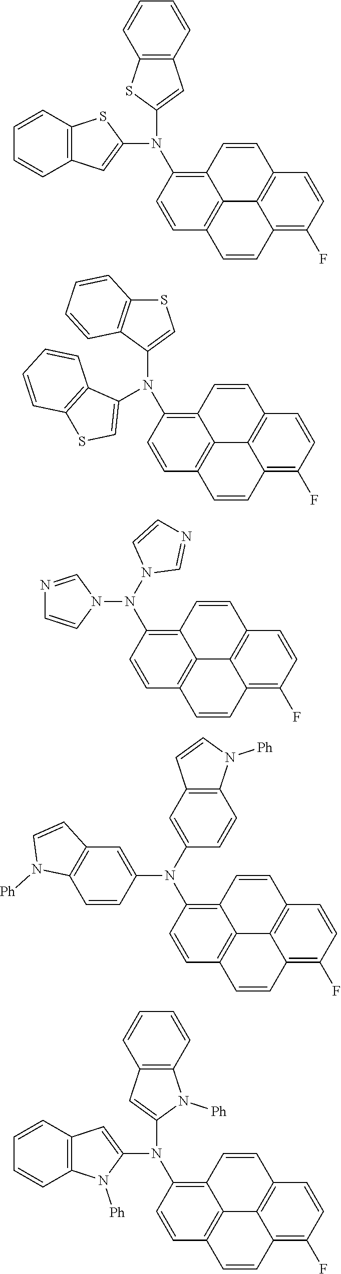

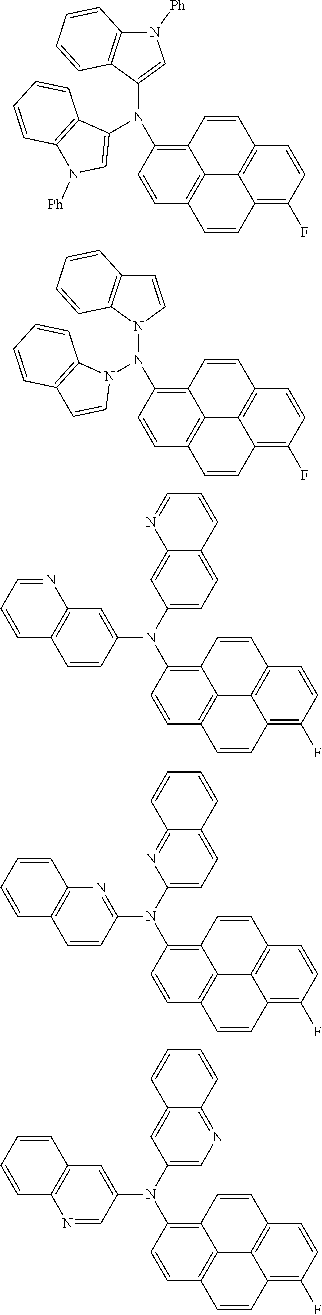

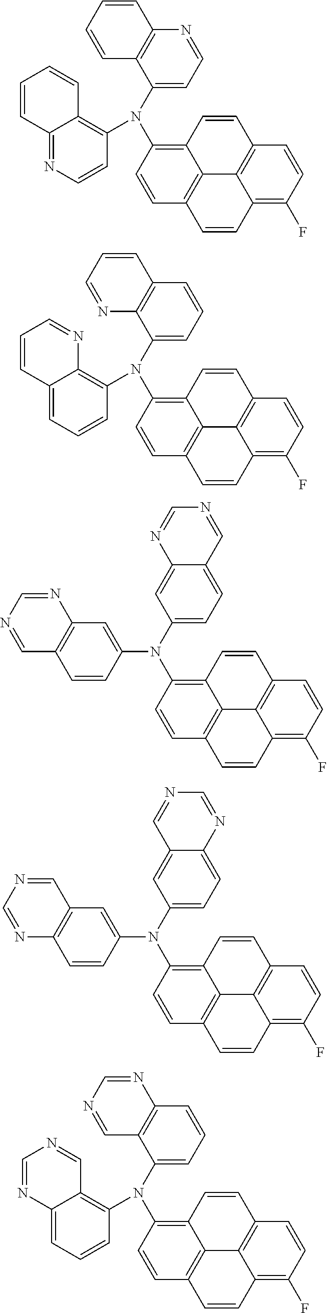

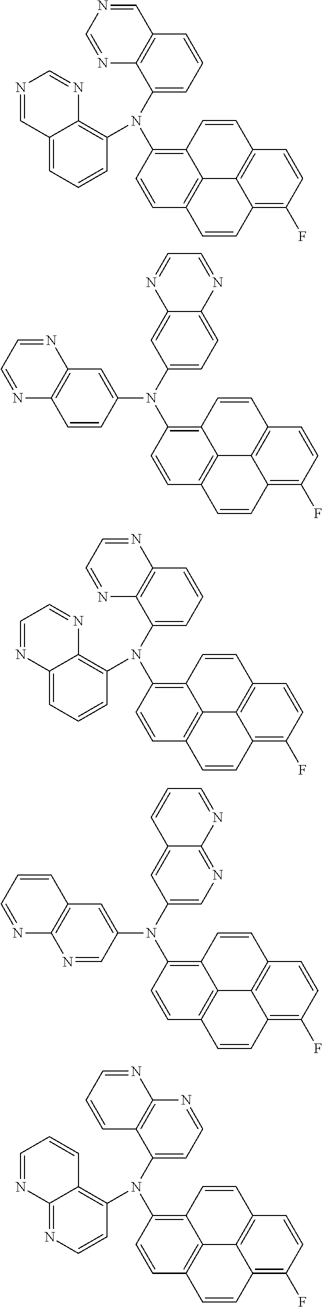

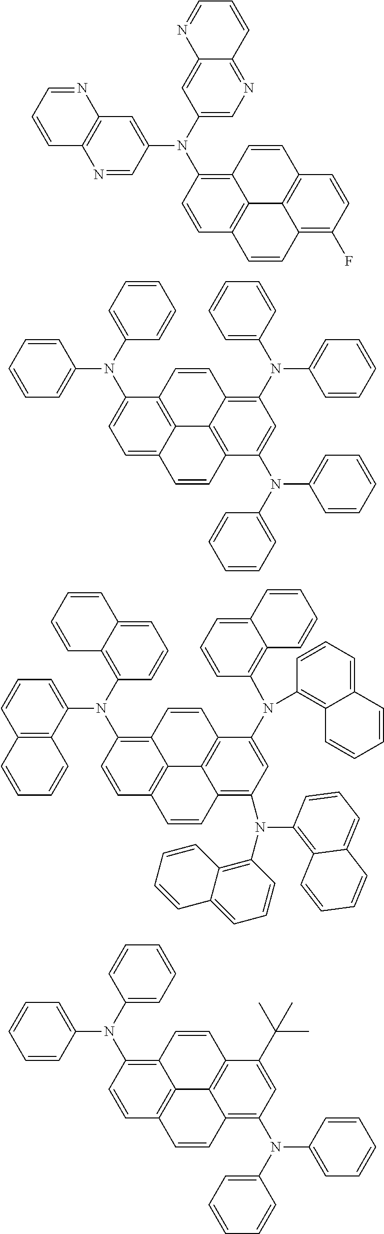

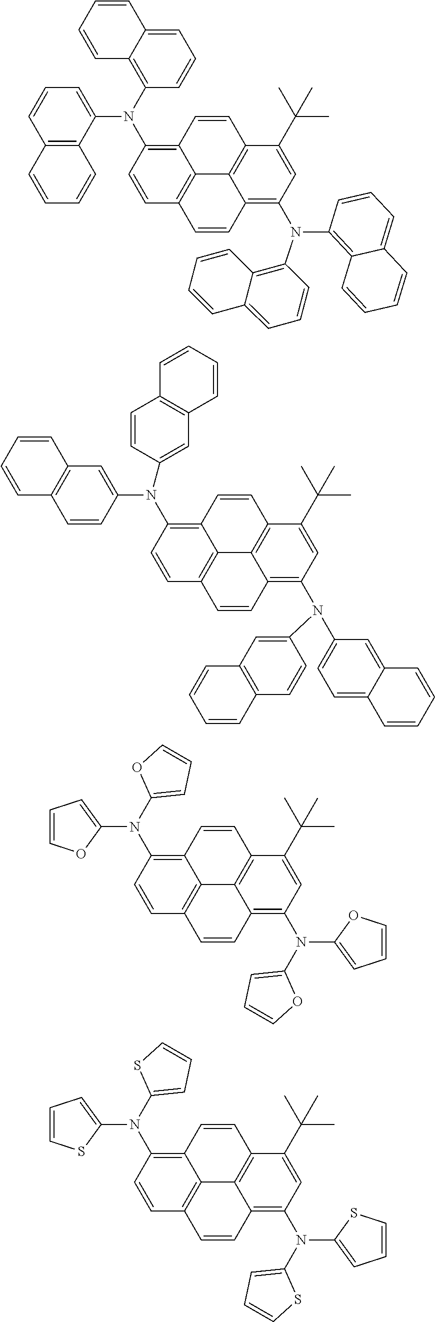

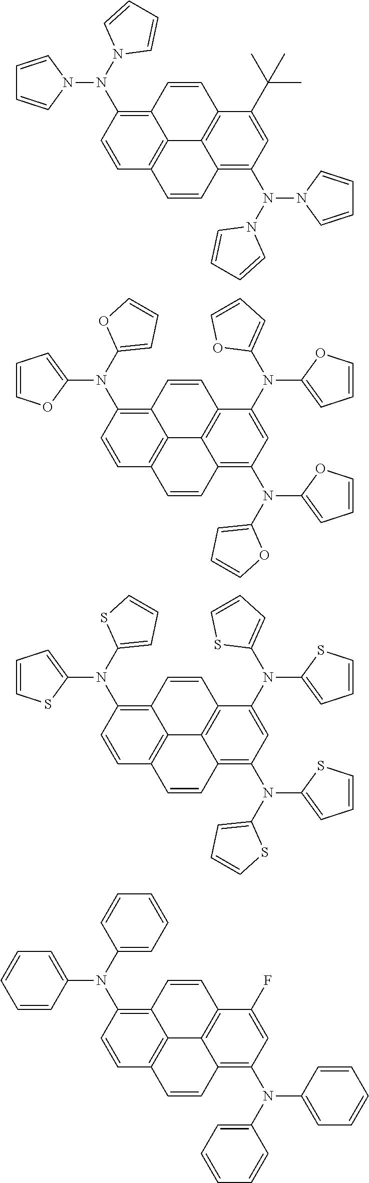

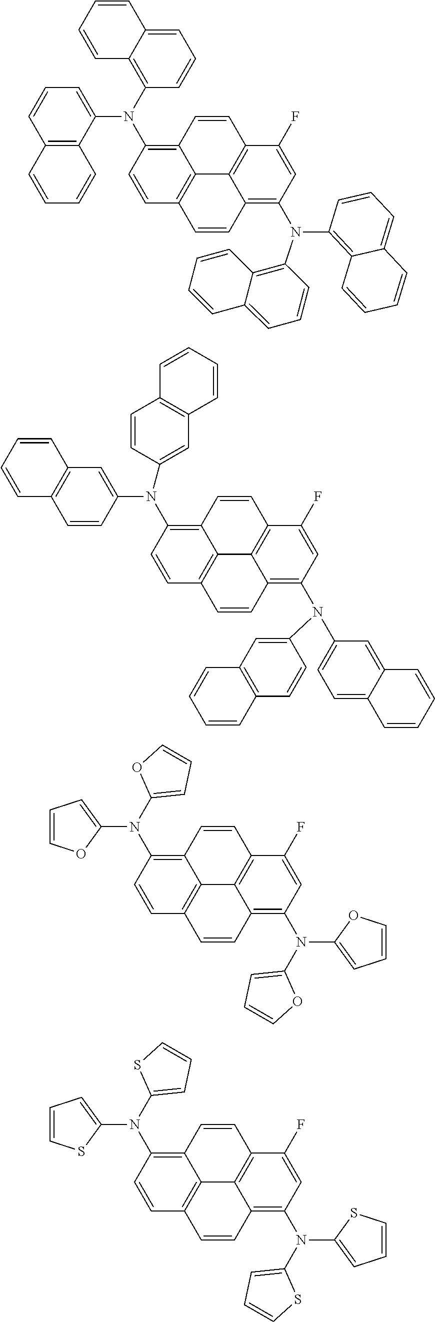

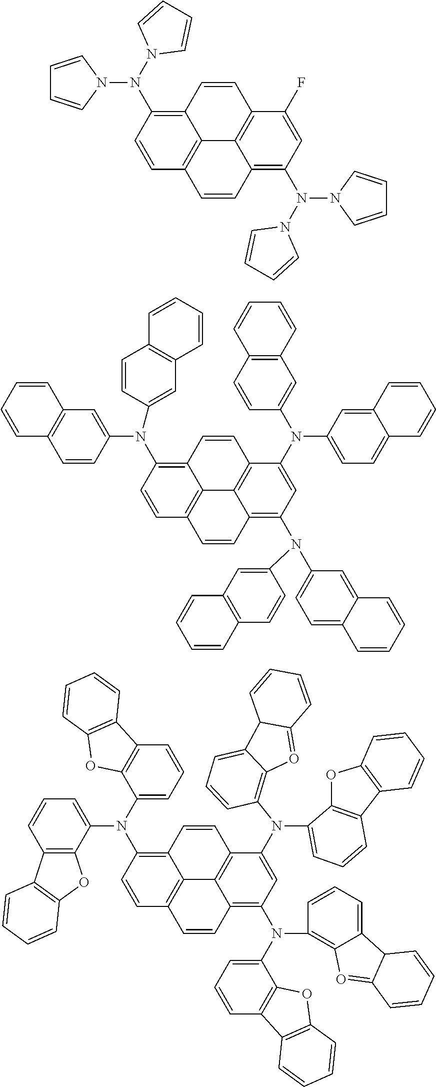

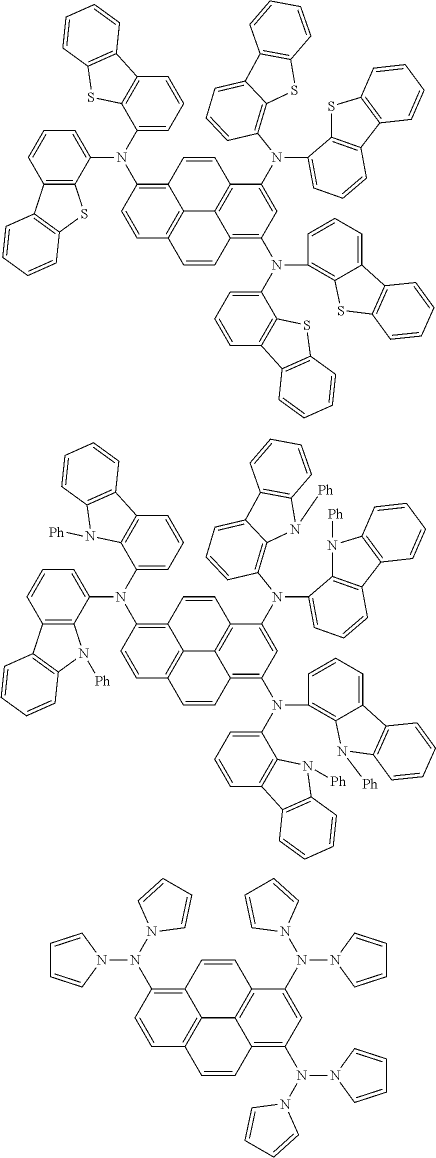

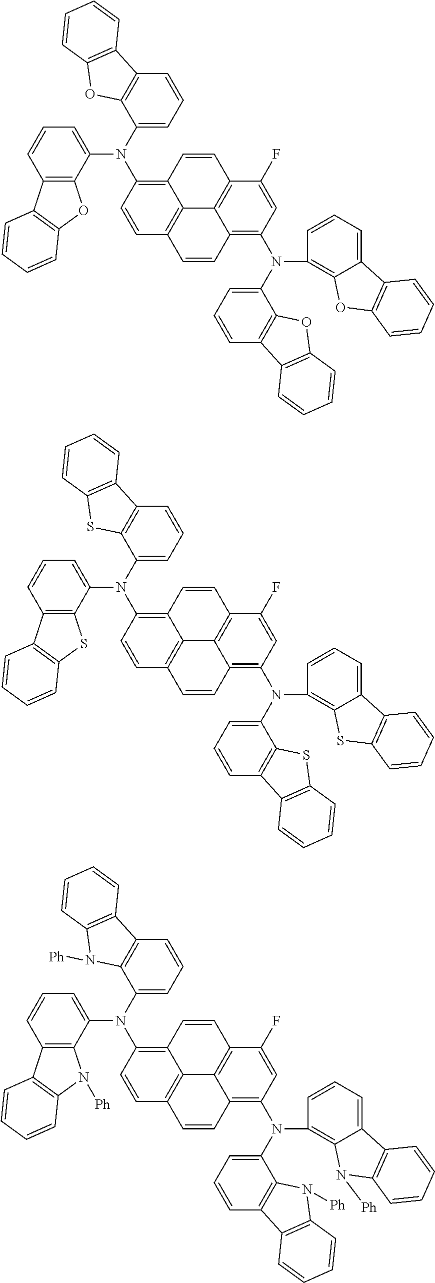 [SMILES: Fc1cc(N(c2cccc3c2oc2ccccc23)c2cccc3c2oc2ccccc23)c2ccc3ccc(N(c4cccc5c4oc4ccccc45)c4cccc5c4oc4ccccc45)c4ccc1c2c34 ; Fc1cc(N(c2cccc3c2sc2ccccc23)c2cccc3c2sc2ccccc23)c2ccc3ccc(N(c4cccc5c4sc4ccccc45)c4cccc5c4sc4ccccc45)c4ccc1c2c34 ; Fc1cc(N(c2cccc3c4ccccc4n(-c4ccccc4)c23)c2cccc3c4ccccc4n(-c4ccccc4)c23)c2ccc3ccc(N(c4cccc5c6ccccc6n(-c6ccccc6)c45)c4cccc5c6ccccc6n(-c6ccccc6)c45)c4ccc1c2c34]

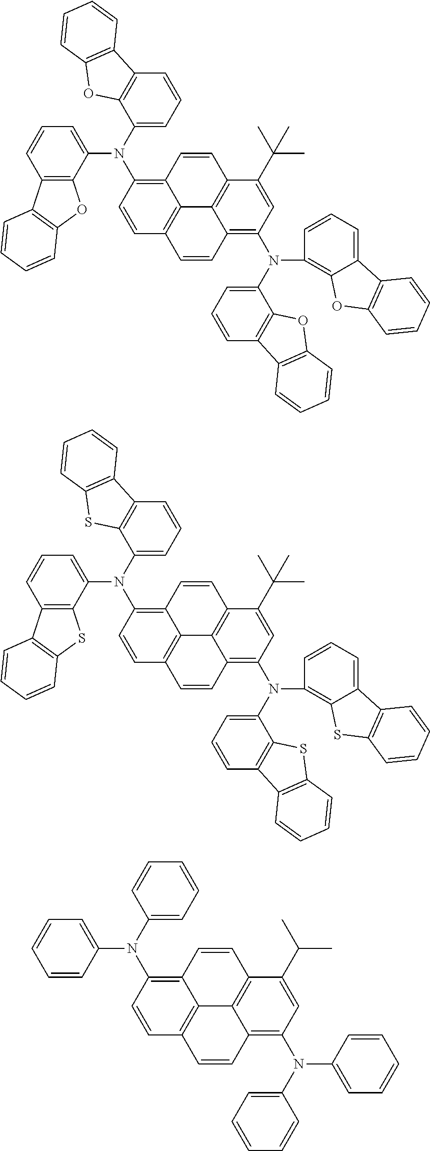 [SMILES: CC(C)(C)c1cc(N(c2cccc3c2oc2ccccc23)c2cccc3c2oc2ccccc23)c2ccc3ccc(N(c4cccc5c4oc4ccccc45)c4cccc5c4oc4ccccc45)c4ccc1c2c34 ; CC(C)(C)c1cc(N(c2cccc3c2sc2ccccc23)c2cccc3c2sc2ccccc23)c2ccc3ccc(N(c4cccc5c4sc4ccccc45)c4cccc5c4sc4ccccc45)c4ccc1c2c34 ; CC(C)c1cc(N(c2ccccc2)c2ccccc2)c2ccc3ccc(N(c4ccccc4)c4ccccc4)c4ccc1c2c34]

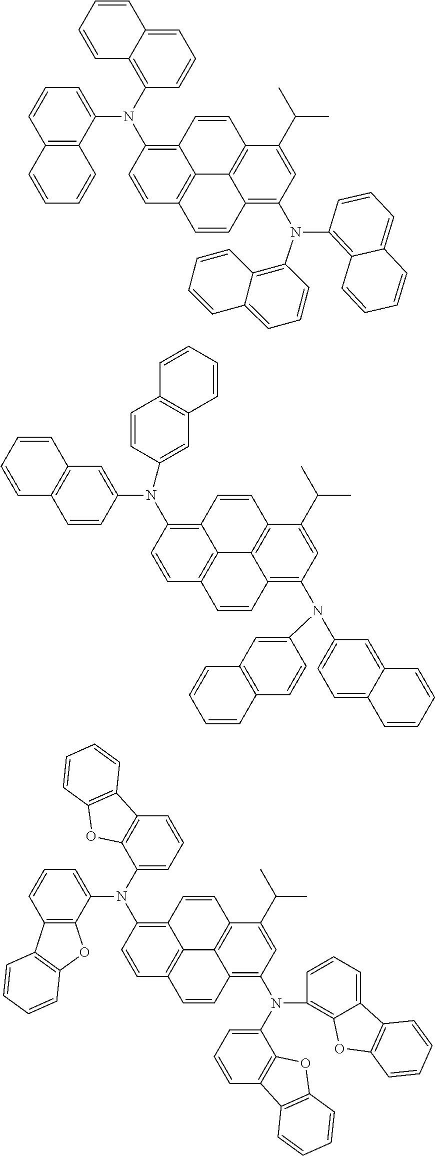

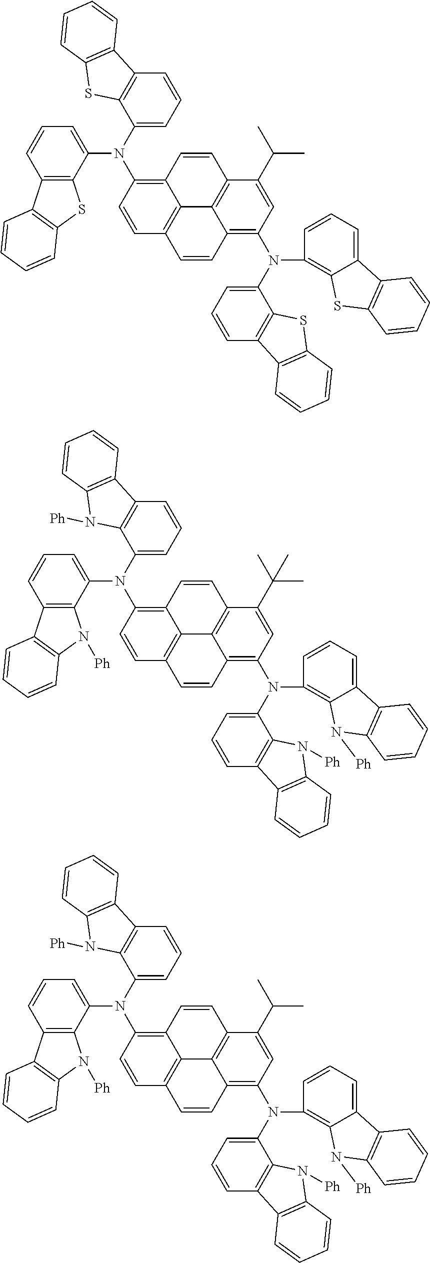 [SMILES: CC(C)c1cc(N(c2cccc3c2sc2ccccc23)c2cccc3c2sc2ccccc23)c2ccc3ccc(N(c4cccc5c4sc4ccccc45)c4cccc5c4sc4ccccc45)c4ccc1c2c34 ; CC(C)(C)c1cc(N(c2cccc3c4ccccc4n(-c4ccccc4)c23)c2cccc3c4ccccc4n(-c4ccccc4)c23)c2ccc3ccc(N(c4cccc5c6ccccc6n(-c6ccccc6)c45)c4cccc5c6ccccc6n(-c6ccccc6)c45)c4ccc1c2c34 ; CC(C)c1cc(N(c2cccc3c4ccccc4n(-c4ccccc4)c23)c2cccc3c4ccccc4n(-c4ccccc4)c23)c2ccc3ccc(N(c4cccc5c6ccccc6n(-c6ccccc6)c45)c4cccc5c6ccccc6n(-c6ccccc6)c45)c4ccc1c2c34]

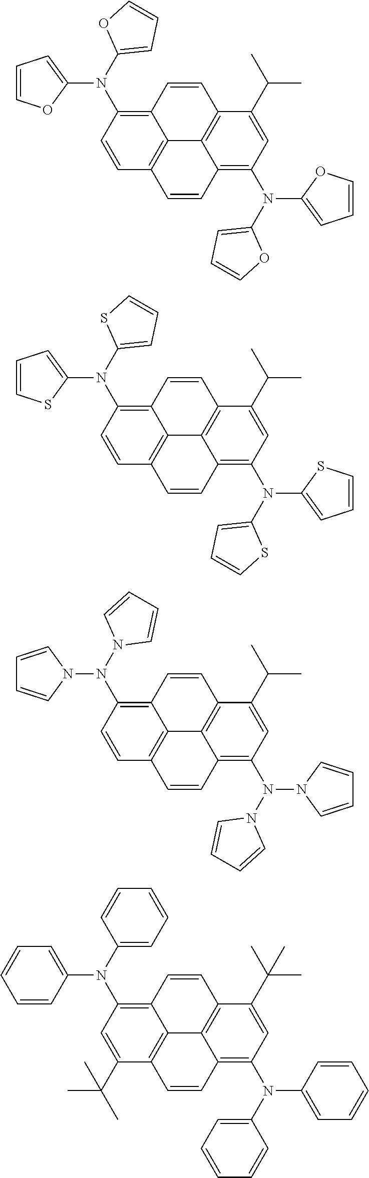 [SMILES: CC(C)c1cc(N(c2ccco2)c2ccco2)c2ccc3ccc(N(c4ccco4)c4ccco4)c4ccc1c2c34 ; CC(C)c1cc(N(c2cccs2)c2cccs2)c2ccc3ccc(N(c4cccs4)c4cccs4)c4ccc1c2c34 ; CC(C)c1cc(N(n2cccc2)n2cccc2)c2ccc3ccc(N(n4cccc4)n4cccc4)c4ccc1c2c34 ; CC(C)(C)c1cc(N(c2ccccc2)c2ccccc2)c2ccc3c(C(C)(C)C)cc(N(c4ccccc4)c4ccccc4)c4ccc1c2c43]

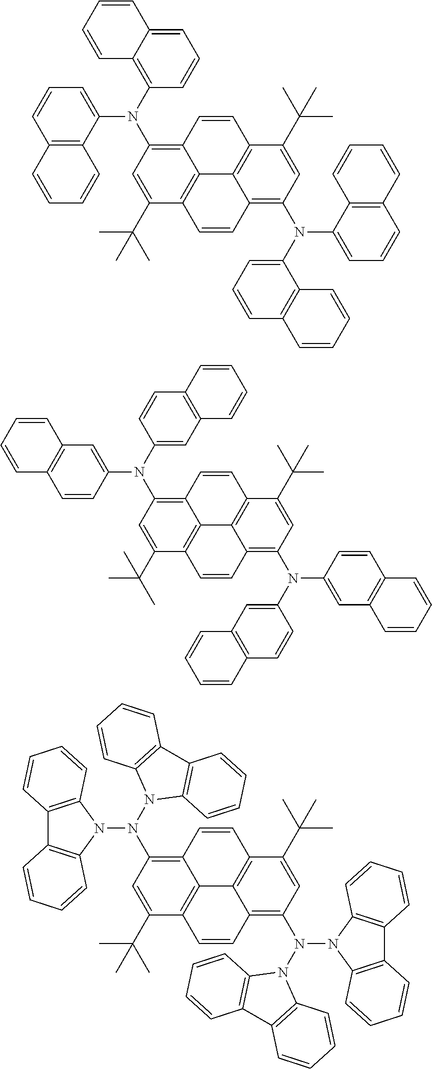 [SMILES: CC(C)(C)c1cc(N(c2cccc3ccccc23)c2cccc3ccccc23)c2ccc3c(C(C)(C)C)cc(N(c4cccc5ccccc45)c4cccc5ccccc45)c4ccc1c2c43 ; CC(C)(C)c1cc(N(c2ccc3ccccc3c2)c2ccc3ccccc3c2)c2ccc3c(C(C)(C)C)cc(N(c4ccc5ccccc5c4)c4ccc5ccccc5c4)c4ccc1c2c43 ; CC(C)(C)c1cc(N(n2c3ccccc3c3ccccc32)n2c3ccccc3c3ccccc32)c2ccc3c(C(C)(C)C)cc(N(n4c5ccccc5c5ccccc54)n4c5ccccc5c5ccccc54)c4ccc1c2c43]

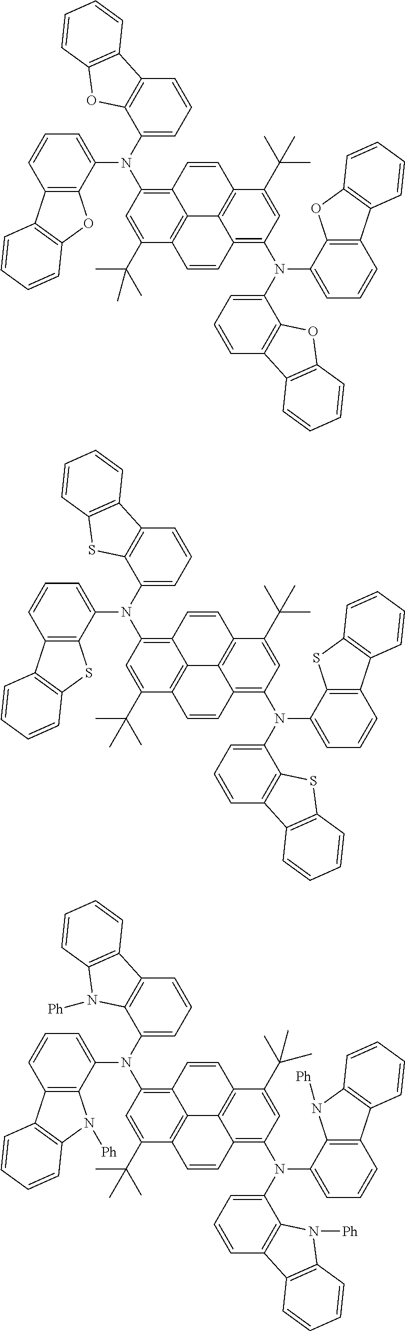 [SMILES: CC(C)(C)c1cc(N(c2cccc3c2oc2ccccc23)c2cccc3c2oc2ccccc23)c2ccc3c(C(C)(C)C)cc(N(c4cccc5c4oc4ccccc45)c4cccc5c4oc4ccccc45)c4ccc1c2c43 ; CC(C)(C)c1cc(N(c2cccc3c2sc2ccccc23)c2cccc3c2sc2ccccc23)c2ccc3c(C(C)(C)C)cc(N(c4cccc5c4sc4ccccc45)c4cccc5c4sc4ccccc45)c4ccc1c2c43 ; CC(C)(C)c1cc(N(c2cccc3c4ccccc4n(-c4ccccc4)c23)c2cccc3c4ccccc4n(-c4ccccc4)c23)c2ccc3c(C(C)(C)C)cc(N(c4cccc5c6ccccc6n(-c6ccccc6)c45)c4cccc5c6ccccc6n(-c6ccccc6)c45)c4ccc1c2c43]

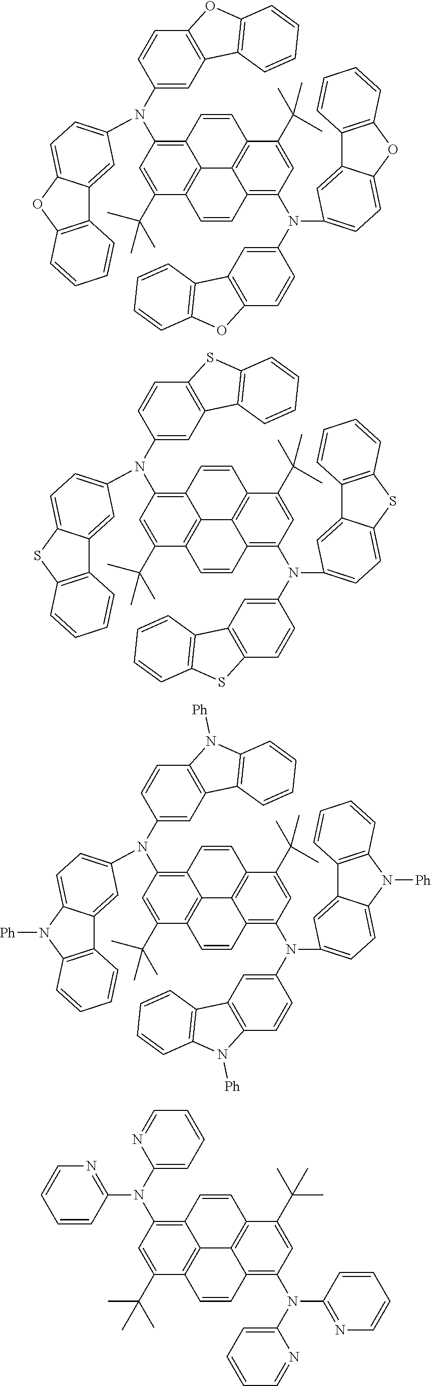

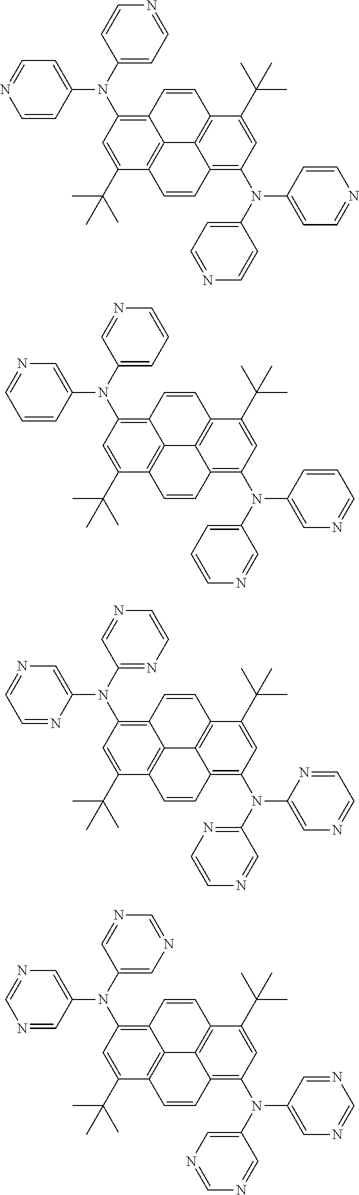 [SMILES: CC(C)(C)c1cc(N(c2ccncc2)c2ccncc2)c2ccc3c(C(C)(C)C)cc(N(c4ccncc4)c4ccncc4)c4ccc1c2c43 ; CC(C)(C)c1cc(N(c2cccnc2)c2cccnc2)c2ccc3c(C(C)(C)C)cc(N(c4cccnc4)c4cccnc4)c4ccc1c2c43 ; CC(C)(C)c1cc(N(c2cnccn2)c2cnccn2)c2ccc3c(C(C)(C)C)cc(N(c4cnccn4)c4cnccn4)c4ccc1c2c43 ; CC(C)(C)c1cc(N(c2cncnc2)c2cncnc2)c2ccc3c(C(C)(C)C)cc(N(c4cncnc4)c4cncnc4)c4ccc1c2c43]

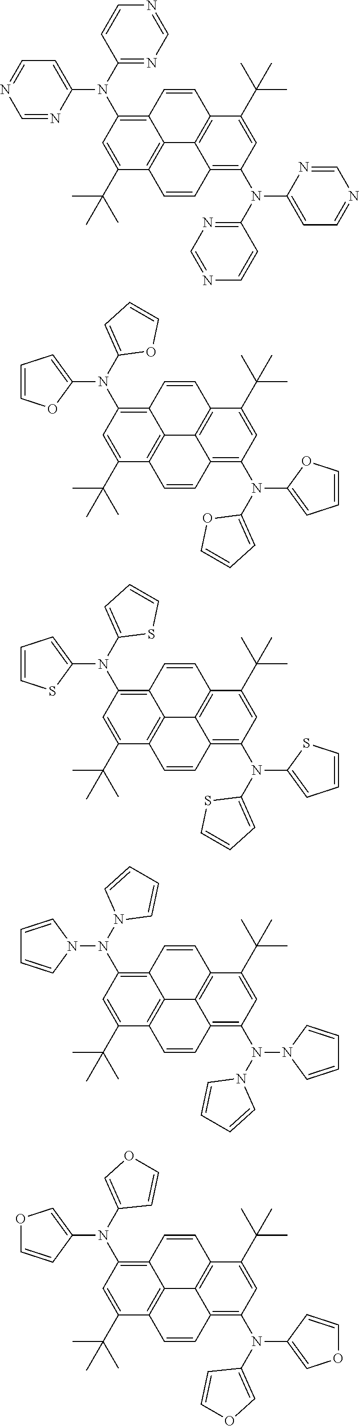 [SMILES: CC(C)(C)c1cc(N(c2ccncn2)c2ccncn2)c2ccc3c(C(C)(C)C)cc(N(c4ccncn4)c4ccncn4)c4ccc1c2c43 ; CC(C)(C)c1cc(N(c2ccco2)c2ccco2)c2ccc3c(C(C)(C)C)cc(N(c4ccco4)c4ccco4)c4ccc1c2c43 ; CC(C)(C)c1cc(N(c2cccs2)c2cccs2)c2ccc3c(C(C)(C)C)cc(N(c4cccs4)c4cccs4)c4ccc1c2c43 ; CC(C)(C)c1cc(N(n2cccc2)n2cccc2)c2ccc3c(C(C)(C)C)cc(N(n4cccc4)n4cccc4)c4ccc1c2c43 ; CC(C)(C)c1cc(N(c2ccoc2)c2ccoc2)c2ccc3c(C(C)(C)C)cc(N(c4ccoc4)c4ccoc4)c4ccc1c2c43]

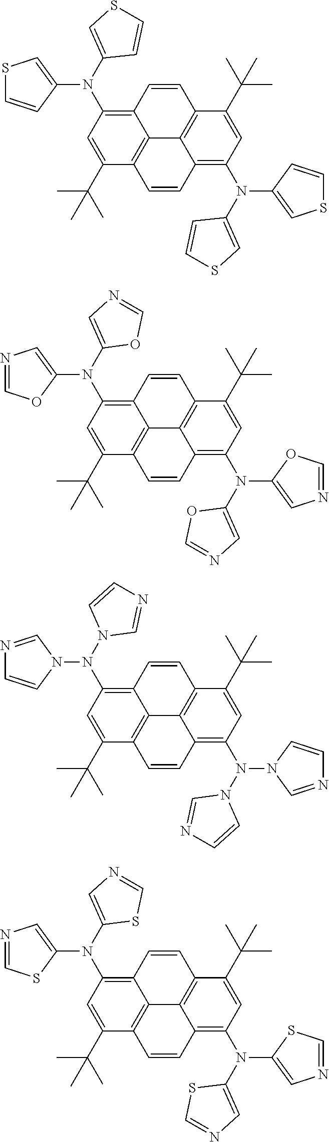 [SMILES: CC(C)(C)c1cc(N(c2ccsc2)c2ccsc2)c2ccc3c(C(C)(C)C)cc(N(c4ccsc4)c4ccsc4)c4ccc1c2c43 ; CC(C)(C)c1cc(N(c2cnco2)c2cnco2)c2ccc3c(C(C)(C)C)cc(N(c4cnco4)c4cnco4)c4ccc1c2c43 ; CC(C)(C)c1cc(N(n2ccnc2)n2ccnc2)c2ccc3c(C(C)(C)C)cc(N(n4ccnc4)n4ccnc4)c4ccc1c2c43 ; CC(C)(C)c1cc(N(c2cncs2)c2cncs2)c2ccc3c(C(C)(C)C)cc(N(c4cncs4)c4cncs4)c4ccc1c2c43]

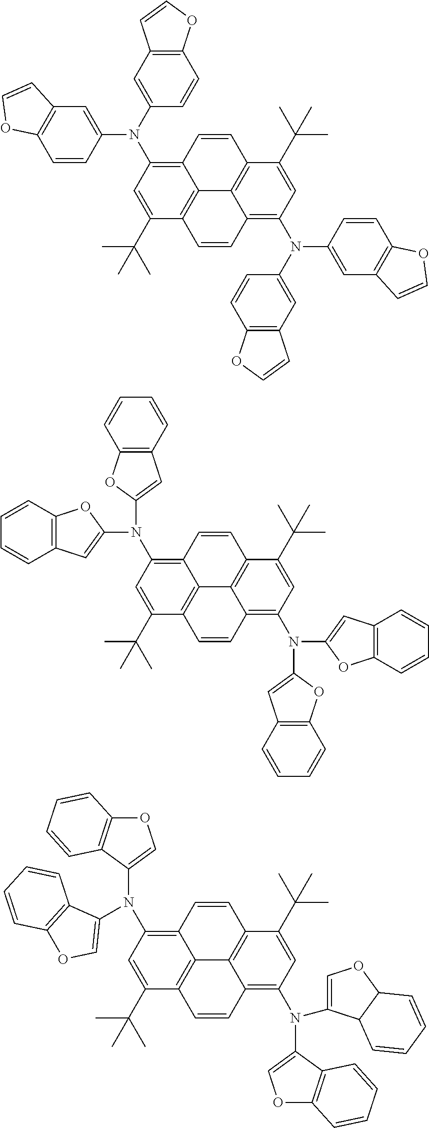 [SMILES: CC(C)(C)c1cc(N(c2ccc3occc3c2)c2ccc3occc3c2)c2ccc3c(C(C)(C)C)cc(N(c4ccc5occc5c4)c4ccc5occc5c4)c4ccc1c2c43 ; CC(C)(C)c1cc(N(c2cc3ccccc3o2)c2cc3ccccc3o2)c2ccc3c(C(C)(C)C)cc(N(c4cc5ccccc5o4)c4cc5ccccc5o4)c4ccc1c2c43 ; CC(C)(C)c1cc(N(C2=COC3C=CC=CC23)c2coc3ccccc23)c2ccc3c(C(C)(C)C)cc(N(c4coc5ccccc45)c4coc5ccccc45)c4ccc1c2c43]

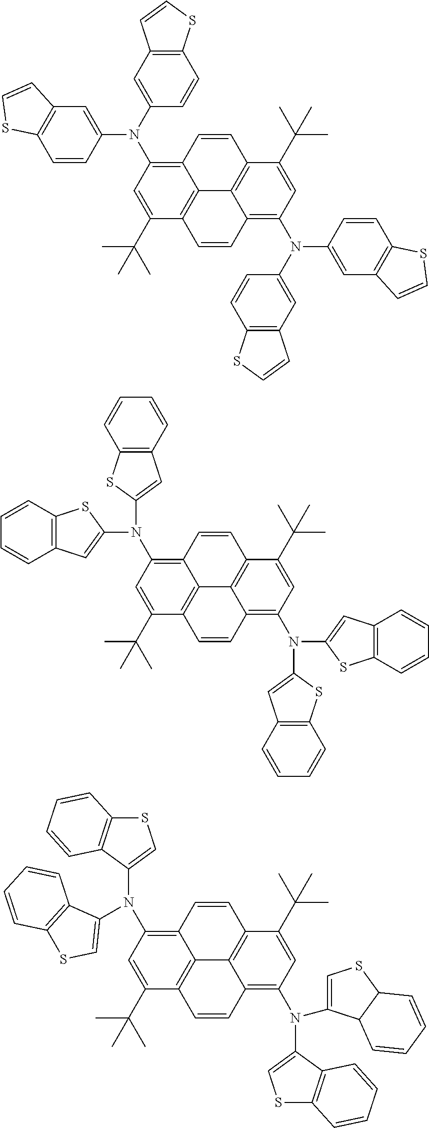 [SMILES: CC(C)(C)c1cc(N(c2ccc3sccc3c2)c2ccc3sccc3c2)c2ccc3c(C(C)(C)C)cc(N(c4ccc5sccc5c4)c4ccc5sccc5c4)c4ccc1c2c43 ; CC(C)(C)c1cc(N(c2cc3ccccc3s2)c2cc3ccccc3s2)c2ccc3c(C(C)(C)C)cc(N(c4cc5ccccc5s4)c4cc5ccccc5s4)c4ccc1c2c43 ; CC(C)(C)c1cc(N(C2=CSC3C=CC=CC23)c2csc3ccccc23)c2ccc3c(C(C)(C)C)cc(N(c4csc5ccccc45)c4csc5ccccc45)c4ccc1c2c43]

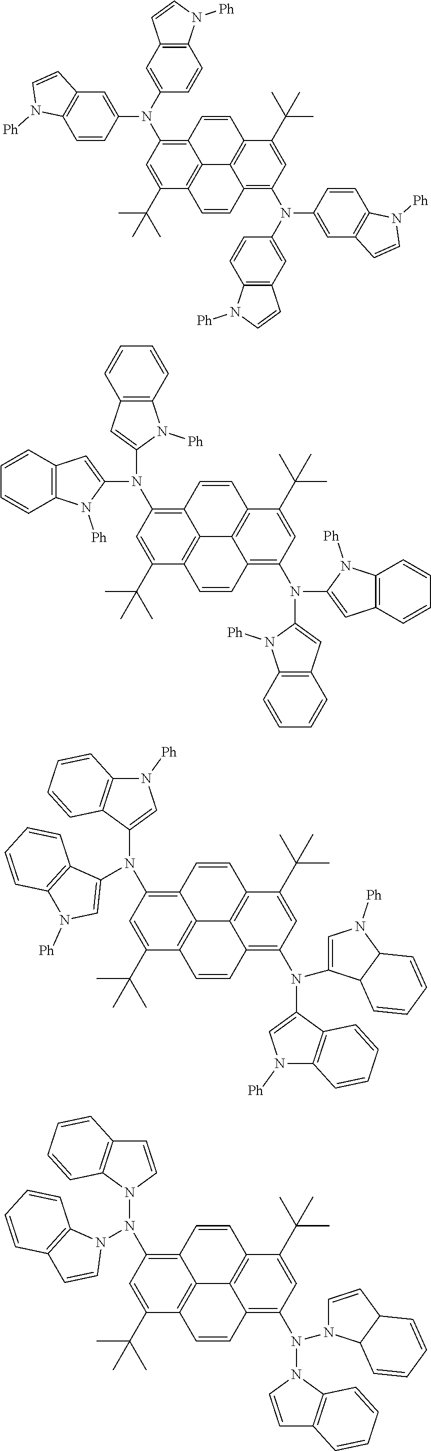

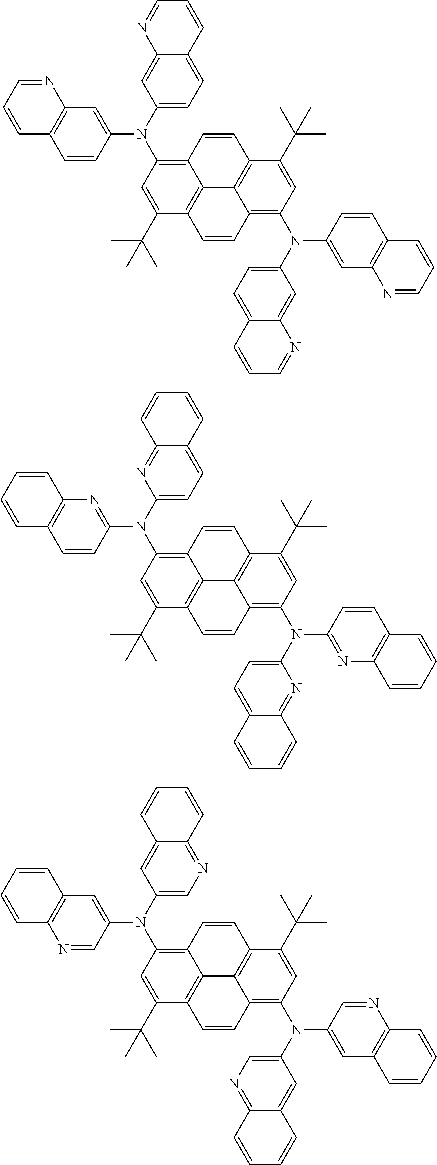 [SMILES: CC(C)(C)c1cc(N(c2ccc3cccnc3c2)c2ccc3cccnc3c2)c2ccc3c(C(C)(C)C)cc(N(c4ccc5cccnc5c4)c4ccc5cccnc5c4)c4ccc1c2c43 ; CC(C)(C)c1cc(N(c2ccc3ccccc3n2)c2ccc3ccccc3n2)c2ccc3c(C(C)(C)C)cc(N(c4ccc5ccccc5n4)c4ccc5ccccc5n4)c4ccc1c2c43 ; CC(C)(C)c1cc(N(c2cnc3ccccc3c2)c2cnc3ccccc3c2)c2ccc3c(C(C)(C)C)cc(N(c4cnc5ccccc5c4)c4cnc5ccccc5c4)c4ccc1c2c43]

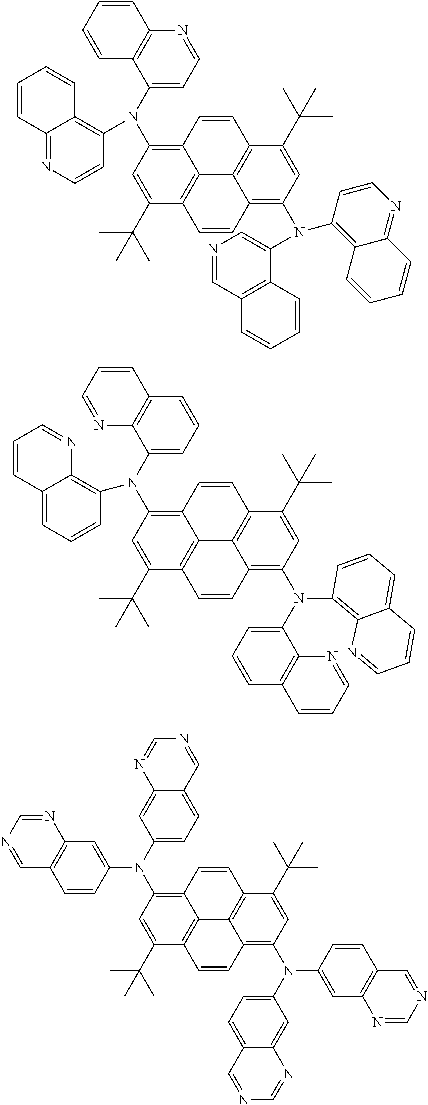 [SMILES: CC(C)(C)c1cc(N(c2cncc3ccccc23)c2ccnc3ccccc23)c2ccc3c(C(C)(C)C)cc(N(c4ccnc5ccccc45)c4ccnc5ccccc45)c4ccc1c2c43 ; CC(C)(C)c1cc(N(c2cccc3cccnc23)c2cccc3cccnc23)c2ccc3c(C(C)(C)C)cc(N(c4cccc5cccnc45)c4cccc5cccnc45)c4ccc1c2c43 ; CC(C)(C)c1cc(N(c2ccc3cncnc3c2)c2ccc3cncnc3c2)c2ccc3c(C(C)(C)C)cc(N(c4ccc5cncnc5c4)c4ccc5cncnc5c4)c4ccc1c2c43]

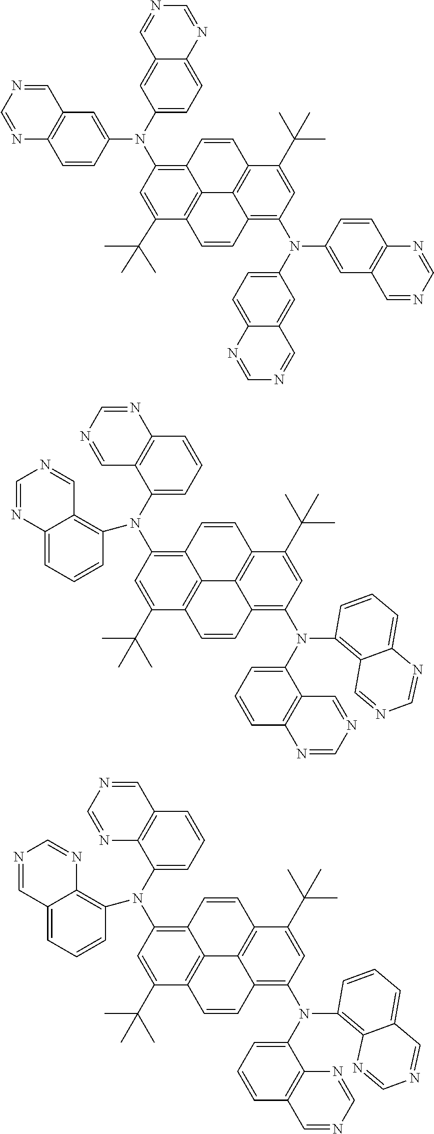 [SMILES: CC(C)(C)c1cc(N(c2ccc3ncncc3c2)c2ccc3ncncc3c2)c2ccc3c(C(C)(C)C)cc(N(c4ccc5ncncc5c4)c4ccc5ncncc5c4)c4ccc1c2c43 ; CC(C)(C)c1cc(N(c2cccc3ncncc23)c2cccc3ncncc23)c2ccc3c(C(C)(C)C)cc(N(c4cccc5ncncc45)c4cccc5ncncc45)c4ccc1c2c43 ; CC(C)(C)c1cc(N(c2cccc3cncnc23)c2cccc3cncnc23)c2ccc3c(C(C)(C)C)cc(N(c4cccc5cncnc45)c4cccc5cncnc45)c4ccc1c2c43]

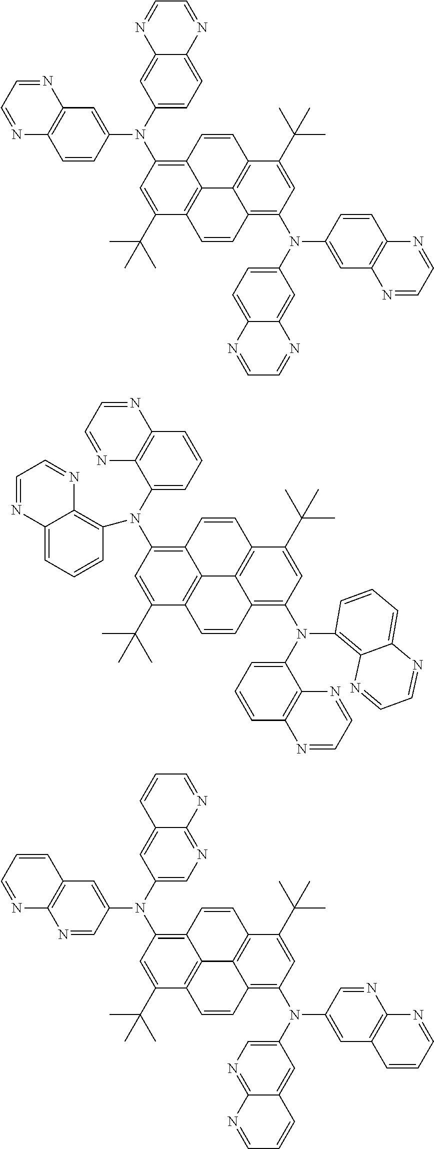 [SMILES: CC(C)(C)c1cc(N(c2ccc3nccnc3c2)c2ccc3nccnc3c2)c2ccc3c(C(C)(C)C)cc(N(c4ccc5nccnc5c4)c4ccc5nccnc5c4)c4ccc1c2c43 ; CC(C)(C)c1cc(N(c2cccc3nccnc23)c2cccc3nccnc23)c2ccc3c(C(C)(C)C)cc(N(c4cccc5nccnc45)c4cccc5nccnc45)c4ccc1c2c43 ; CC(C)(C)c1cc(N(c2cnc3ncccc3c2)c2cnc3ncccc3c2)c2ccc3c(C(C)(C)C)cc(N(c4cnc5ncccc5c4)c4cnc5ncccc5c4)c4ccc1c2c43]

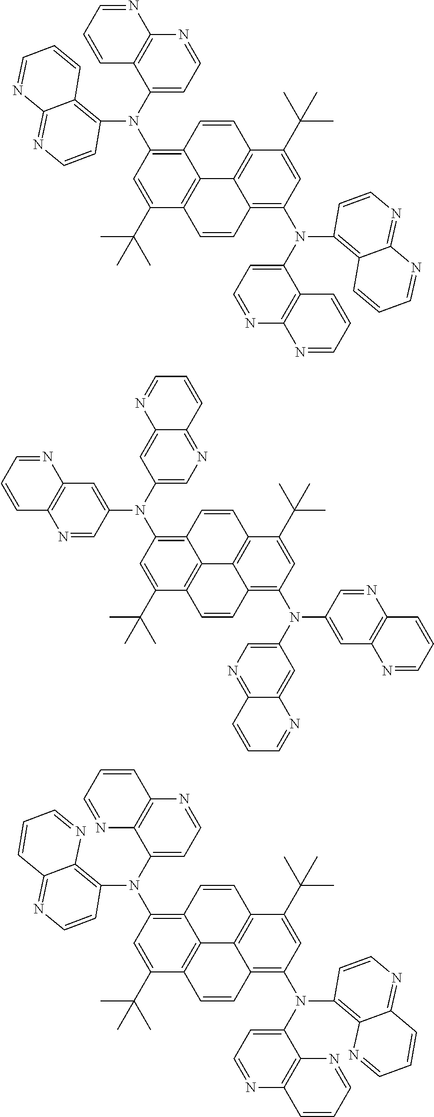 [SMILES: CC(C)(C)c1cc(N(c2ccnc3ncccc23)c2ccnc3ncccc23)c2ccc3c(C(C)(C)C)cc(N(c4ccnc5ncccc45)c4ccnc5ncccc45)c4ccc1c2c43 ; CC(C)(C)c1cc(N(c2cnc3cccnc3c2)c2cnc3cccnc3c2)c2ccc3c(C(C)(C)C)cc(N(c4cnc5cccnc5c4)c4cnc5cccnc5c4)c4ccc1c2c43 ; CC(C)(C)c1cc(N(c2ccnc3cccnc23)c2ccnc3cccnc23)c2ccc3c(C(C)(C)C)cc(N(c4ccnc5cccnc45)c4ccnc5cccnc45)c4ccc1c2c43]

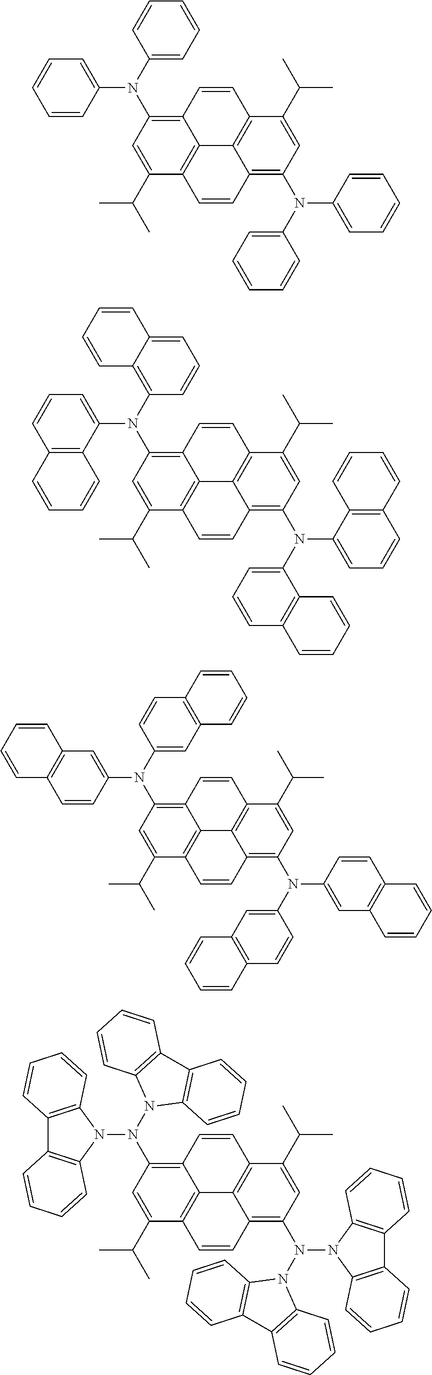 [SMILES: CC(C)c1cc(N(c2ccccc2)c2ccccc2)c2ccc3c(C(C)C)cc(N(c4ccccc4)c4ccccc4)c4ccc1c2c34 ; CC(C)c1cc(N(c2cccc3ccccc23)c2cccc3ccccc23)c2ccc3c(C(C)C)cc(N(c4cccc5ccccc45)c4cccc5ccccc45)c4ccc1c2c34 ; CC(C)c1cc(N(c2ccc3ccccc3c2)c2ccc3ccccc3c2)c2ccc3c(C(C)C)cc(N(c4ccc5ccccc5c4)c4ccc5ccccc5c4)c4ccc1c2c34 ; CC(C)c1cc(N(n2c3ccccc3c3ccccc32)n2c3ccccc3c3ccccc32)c2ccc3c(C(C)C)cc(N(n4c5ccccc5c5ccccc54)n4c5ccccc5c5ccccc54)c4ccc1c2c34]

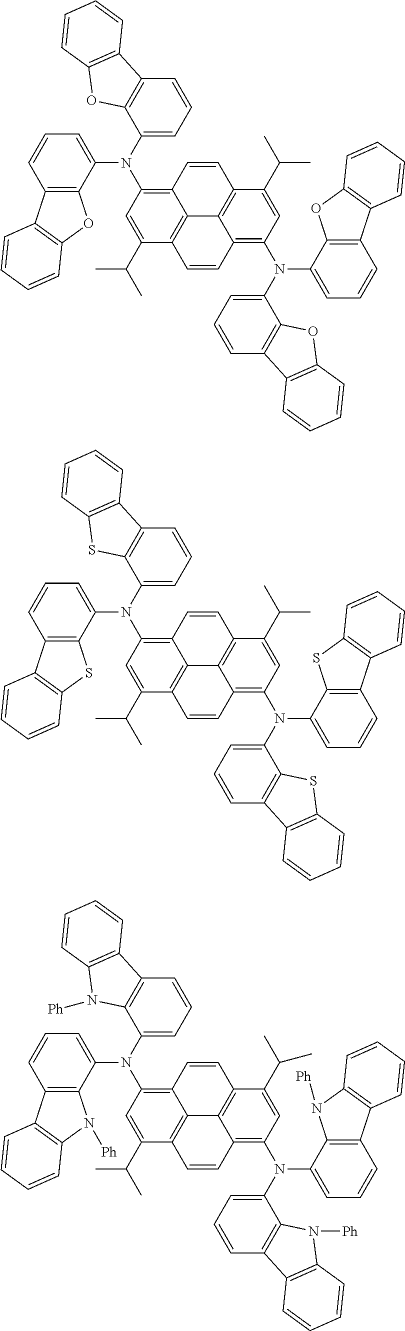 [SMILES: CC(C)c1cc(N(c2cccc3c2oc2ccccc23)c2cccc3c2oc2ccccc23)c2ccc3c(C(C)C)cc(N(c4cccc5c4oc4ccccc45)c4cccc5c4oc4ccccc45)c4ccc1c2c34 ; CC(C)c1cc(N(c2cccc3c2sc2ccccc23)c2cccc3c2sc2ccccc23)c2ccc3c(C(C)C)cc(N(c4cccc5c4sc4ccccc45)c4cccc5c4sc4ccccc45)c4ccc1c2c34 ; CC(C)c1cc(N(c2cccc3c4ccccc4n(-c4ccccc4)c23)c2cccc3c4ccccc4n(-c4ccccc4)c23)c2ccc3c(C(C)C)cc(N(c4cccc5c6ccccc6n(-c6ccccc6)c45)c4cccc5c6ccccc6n(-c6ccccc6)c45)c4ccc1c2c34]

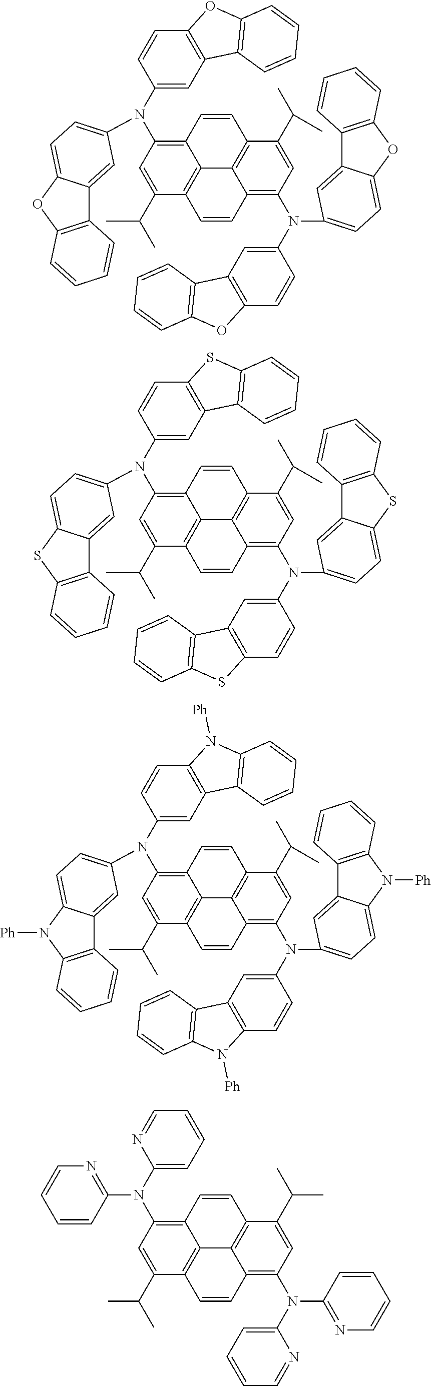

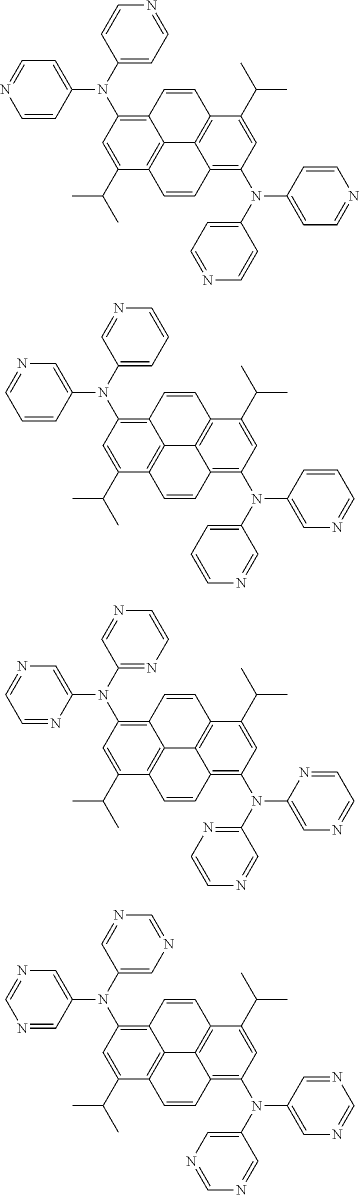 [SMILES: CC(C)c1cc(N(c2ccncc2)c2ccncc2)c2ccc3c(C(C)C)cc(N(c4ccncc4)c4ccncc4)c4ccc1c2c34 ; CC(C)c1cc(N(c2cccnc2)c2cccnc2)c2ccc3c(C(C)C)cc(N(c4cccnc4)c4cccnc4)c4ccc1c2c34 ; CC(C)c1cc(N(c2cnccn2)c2cnccn2)c2ccc3c(C(C)C)cc(N(c4cnccn4)c4cnccn4)c4ccc1c2c34 ; CC(C)c1cc(N(c2cncnc2)c2cncnc2)c2ccc3c(C(C)C)cc(N(c4cncnc4)c4cncnc4)c4ccc1c2c34]

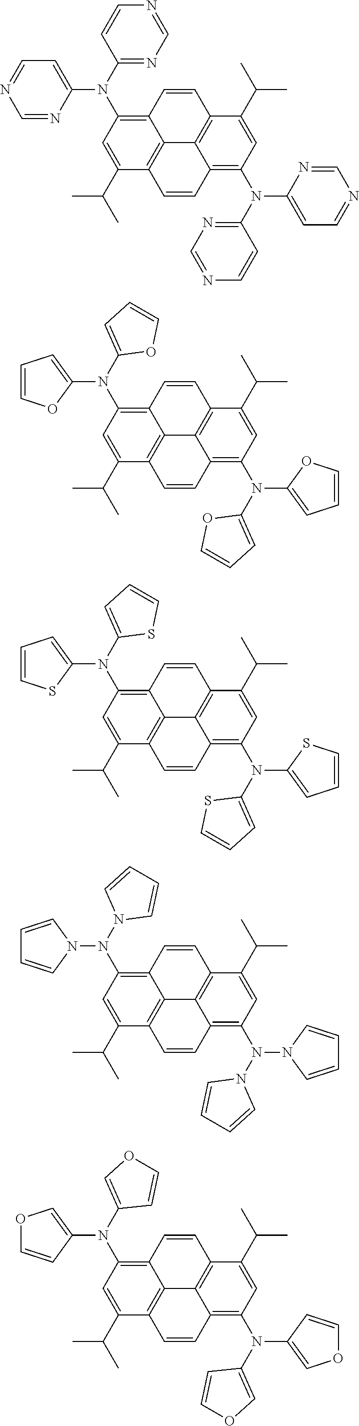 [SMILES: CC(C)c1cc(N(c2ccncn2)c2ccncn2)c2ccc3c(C(C)C)cc(N(c4ccncn4)c4ccncn4)c4ccc1c2c34 ; CC(C)c1cc(N(c2ccco2)c2ccco2)c2ccc3c(C(C)C)cc(N(c4ccco4)c4ccco4)c4ccc1c2c34 ; CC(C)c1cc(N(c2cccs2)c2cccs2)c2ccc3c(C(C)C)cc(N(c4cccs4)c4cccs4)c4ccc1c2c34 ; CC(C)c1cc(N(n2cccc2)n2cccc2)c2ccc3c(C(C)C)cc(N(n4cccc4)n4cccc4)c4ccc1c2c34 ; CC(C)c1cc(N(c2ccoc2)c2ccoc2)c2ccc3c(C(C)C)cc(N(c4ccoc4)c4ccoc4)c4ccc1c2c34]

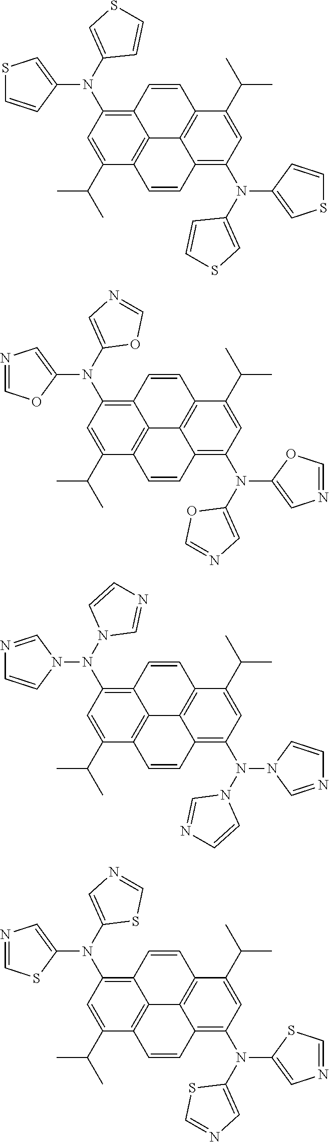 [SMILES: CC(C)c1cc(N(c2ccsc2)c2ccsc2)c2ccc3c(C(C)C)cc(N(c4ccsc4)c4ccsc4)c4ccc1c2c34 ; CC(C)c1cc(N(c2cnco2)c2cnco2)c2ccc3c(C(C)C)cc(N(c4cnco4)c4cnco4)c4ccc1c2c34 ; CC(C)c1cc(N(n2ccnc2)n2ccnc2)c2ccc3c(C(C)C)cc(N(n4ccnc4)n4ccnc4)c4ccc1c2c34 ; CC(C)c1cc(N(c2cncs2)c2cncs2)c2ccc3c(C(C)C)cc(N(c4cncs4)c4cncs4)c4ccc1c2c34]

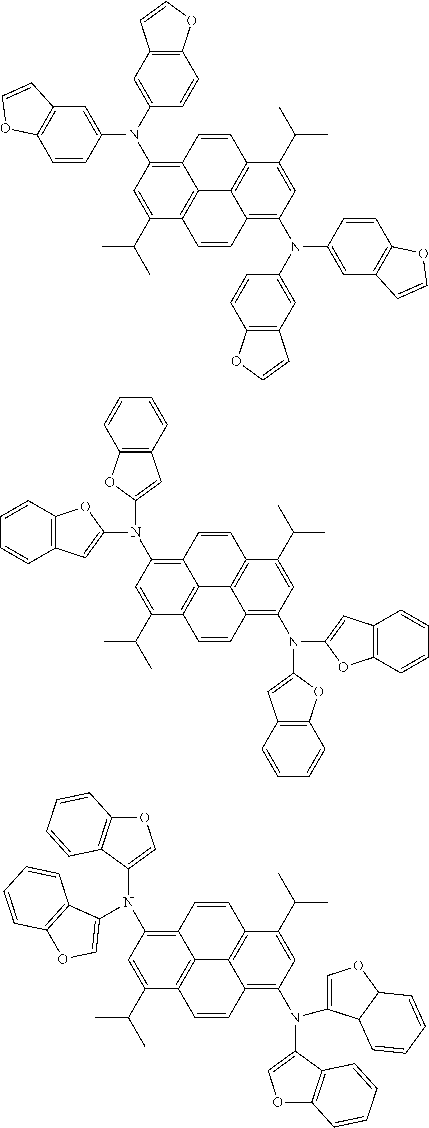 [SMILES: CC(C)c1cc(N(c2ccc3occc3c2)c2ccc3occc3c2)c2ccc3c(C(C)C)cc(N(c4ccc5occc5c4)c4ccc5occc5c4)c4ccc1c2c34 ; CC(C)c1cc(N(c2cc3ccccc3o2)c2cc3ccccc3o2)c2ccc3c(C(C)C)cc(N(c4cc5ccccc5o4)c4cc5ccccc5o4)c4ccc1c2c34 ; CC(C)c1cc(N(C2=COC3C=CC=CC23)c2coc3ccccc23)c2ccc3c(C(C)C)cc(N(c4coc5ccccc45)c4coc5ccccc45)c4ccc1c2c34]

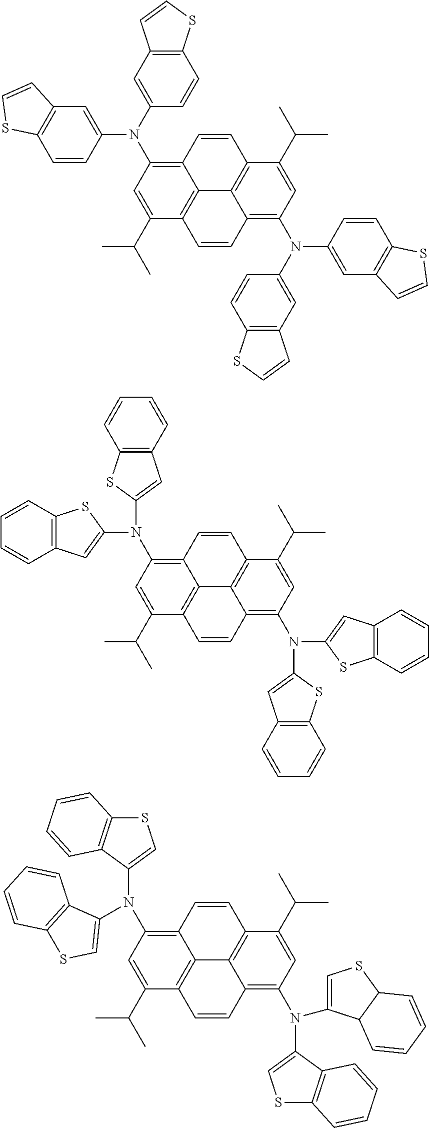 [SMILES: CC(C)c1cc(N(c2ccc3sccc3c2)c2ccc3sccc3c2)c2ccc3c(C(C)C)cc(N(c4ccc5sccc5c4)c4ccc5sccc5c4)c4ccc1c2c34 ; CC(C)c1cc(N(c2cc3ccccc3s2)c2cc3ccccc3s2)c2ccc3c(C(C)C)cc(N(c4cc5ccccc5s4)c4cc5ccccc5s4)c4ccc1c2c34 ; CC(C)c1cc(N(C2=CSC3C=CC=CC23)c2csc3ccccc23)c2ccc3c(C(C)C)cc(N(c4csc5ccccc45)c4csc5ccccc45)c4ccc1c2c34]

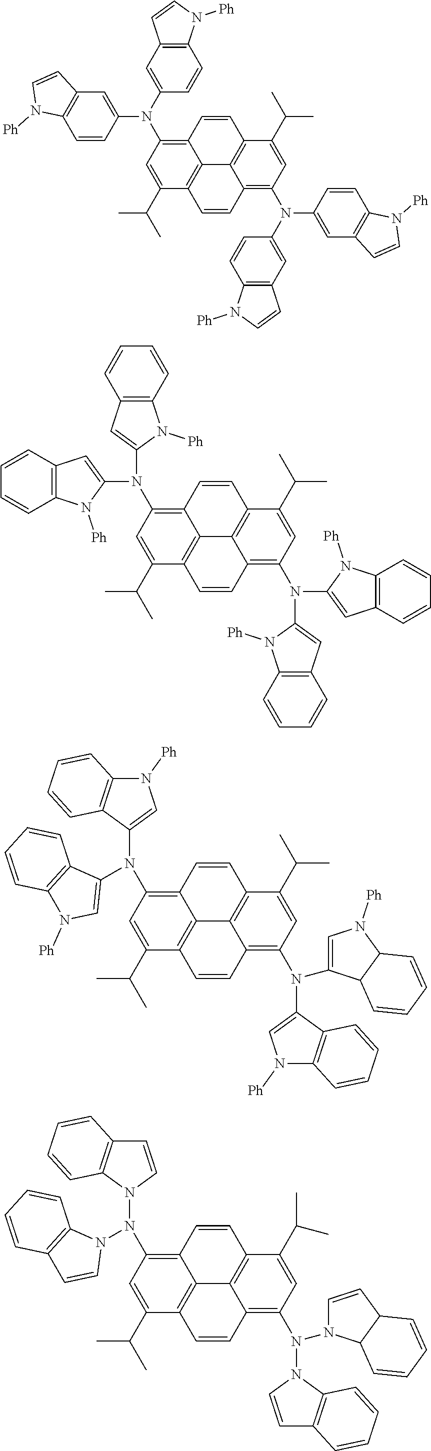

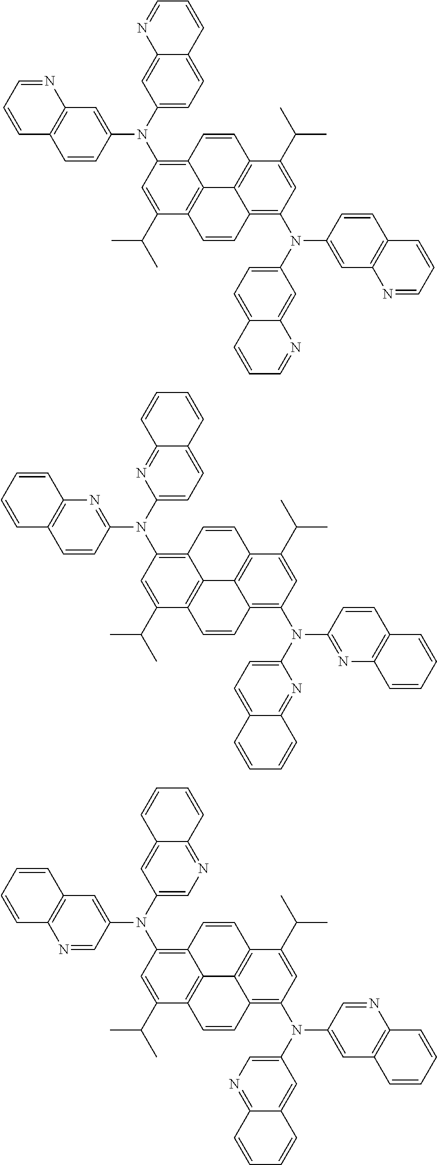 [SMILES: CC(C)c1cc(N(c2ccc3cccnc3c2)c2ccc3cccnc3c2)c2ccc3c(C(C)C)cc(N(c4ccc5cccnc5c4)c4ccc5cccnc5c4)c4ccc1c2c34 ; CC(C)c1cc(N(c2ccc3ccccc3n2)c2ccc3ccccc3n2)c2ccc3c(C(C)C)cc(N(c4ccc5ccccc5n4)c4ccc5ccccc5n4)c4ccc1c2c34 ; CC(C)c1cc(N(c2cnc3ccccc3c2)c2cnc3ccccc3c2)c2ccc3c(C(C)C)cc(N(c4cnc5ccccc5c4)c4cnc5ccccc5c4)c4ccc1c2c34]

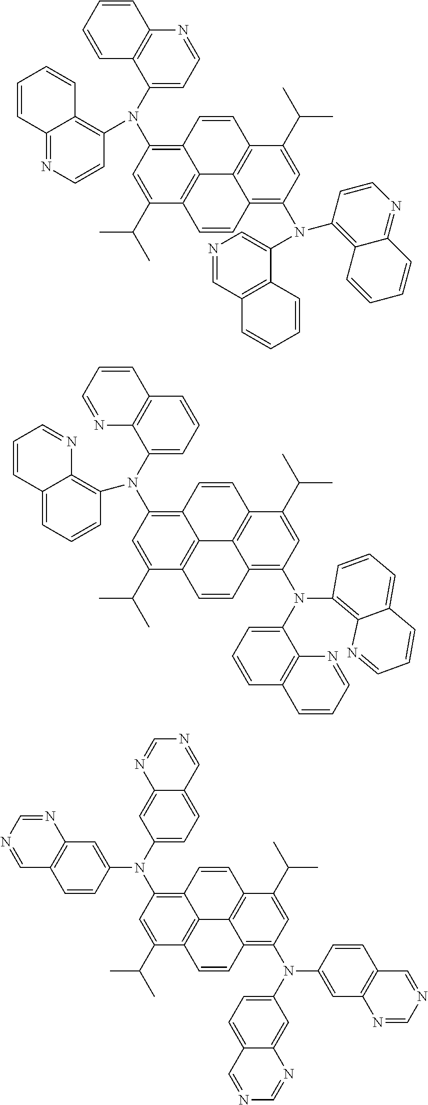 [SMILES: CC(C)c1cc(N(c2cncc3ccccc23)c2ccnc3ccccc23)c2ccc3c(C(C)C)cc(N(c4ccnc5ccccc45)c4ccnc5ccccc45)c4ccc1c2c34 ; CC(C)c1cc(N(c2cccc3cccnc23)c2cccc3cccnc23)c2ccc3c(C(C)C)cc(N(c4cccc5cccnc45)c4cccc5cccnc45)c4ccc1c2c34 ; CC(C)c1cc(N(c2ccc3cncnc3c2)c2ccc3cncnc3c2)c2ccc3c(C(C)C)cc(N(c4ccc5cncnc5c4)c4ccc5cncnc5c4)c4ccc1c2c34]

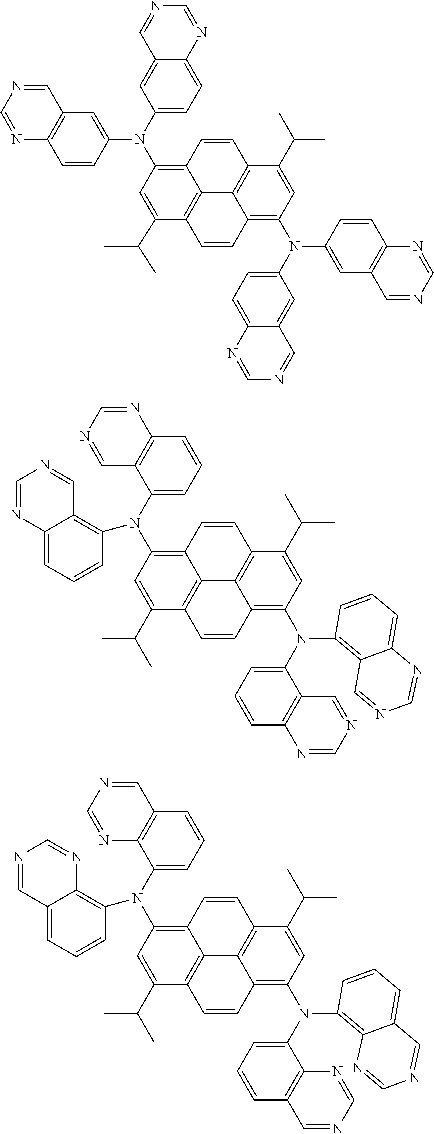 [SMILES: CC(C)c1cc(N(c2ccc3ncncc3c2)c2ccc3ncncc3c2)c2ccc3c(C(C)C)cc(N(c4ccc5ncncc5c4)c4ccc5ncncc5c4)c4ccc1c2c34 ; CC(C)c1cc(N(c2cccc3ncncc23)c2cccc3ncncc23)c2ccc3c(C(C)C)cc(N(c4cccc5ncncc45)c4cccc5ncncc45)c4ccc1c2c34 ; CC(C)c1cc(N(c2cccc3cncnc23)c2cccc3cncnc23)c2ccc3c(C(C)C)cc(N(c4cccc5cncnc45)c4cccc5cncnc45)c4ccc1c2c34]

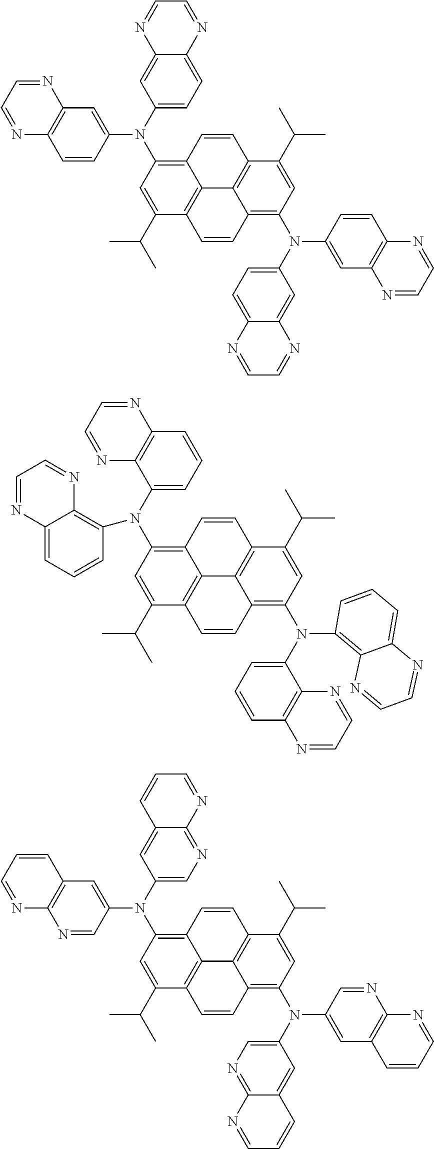 [SMILES: CC(C)c1cc(N(c2ccc3nccnc3c2)c2ccc3nccnc3c2)c2ccc3c(C(C)C)cc(N(c4ccc5nccnc5c4)c4ccc5nccnc5c4)c4ccc1c2c34 ; CC(C)c1cc(N(c2cccc3nccnc23)c2cccc3nccnc23)c2ccc3c(C(C)C)cc(N(c4cccc5nccnc45)c4cccc5nccnc45)c4ccc1c2c34 ; CC(C)c1cc(N(c2cnc3ncccc3c2)c2cnc3ncccc3c2)c2ccc3c(C(C)C)cc(N(c4cnc5ncccc5c4)c4cnc5ncccc5c4)c4ccc1c2c34]

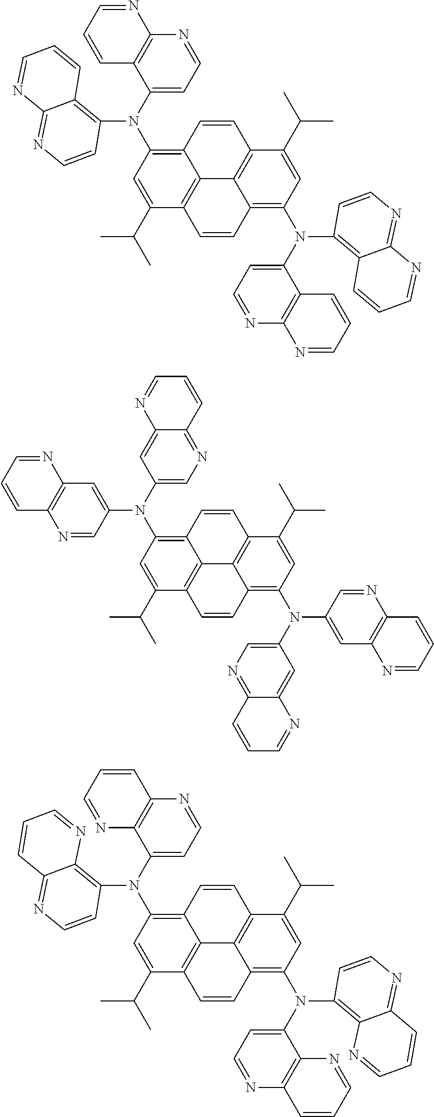 [SMILES: CC(C)c1cc(N(c2ccnc3ncccc23)c2ccnc3ncccc23)c2ccc3c(C(C)C)cc(N(c4ccnc5ncccc45)c4ccnc5ncccc45)c4ccc1c2c34 ; CC(C)c1cc(N(c2cnc3cccnc3c2)c2cnc3cccnc3c2)c2ccc3c(C(C)C)cc(N(c4cnc5cccnc5c4)c4cnc5cccnc5c4)c4ccc1c2c34 ; CC(C)c1cc(N(c2ccnc3cccnc23)c2ccnc3cccnc23)c2ccc3c(C(C)C)cc(N(c4ccnc5cccnc45)c4ccnc5cccnc45)c4ccc1c2c34]

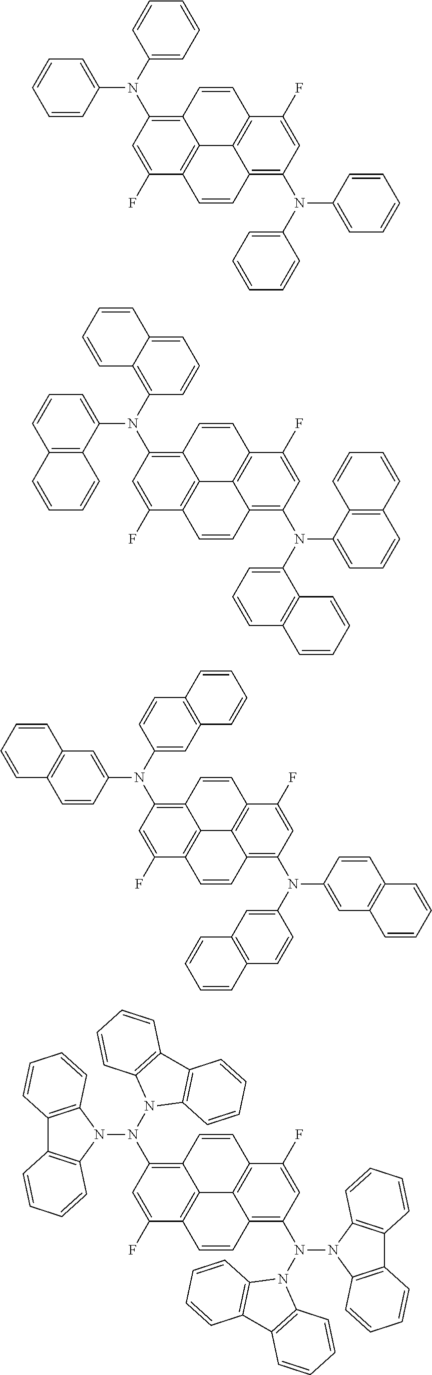 [SMILES: Fc1cc(N(c2ccccc2)c2ccccc2)c2ccc3c(F)cc(N(c4ccccc4)c4ccccc4)c4ccc1c2c34 ; Fc1cc(N(c2cccc3ccccc23)c2cccc3ccccc23)c2ccc3c(F)cc(N(c4cccc5ccccc45)c4cccc5ccccc45)c4ccc1c2c34 ; Fc1cc(N(c2ccc3ccccc3c2)c2ccc3ccccc3c2)c2ccc3c(F)cc(N(c4ccc5ccccc5c4)c4ccc5ccccc5c4)c4ccc1c2c34 ; Fc1cc(N(n2c3ccccc3c3ccccc32)n2c3ccccc3c3ccccc32)c2ccc3c(F)cc(N(n4c5ccccc5c5ccccc54)n4c5ccccc5c5ccccc54)c4ccc1c2c34]

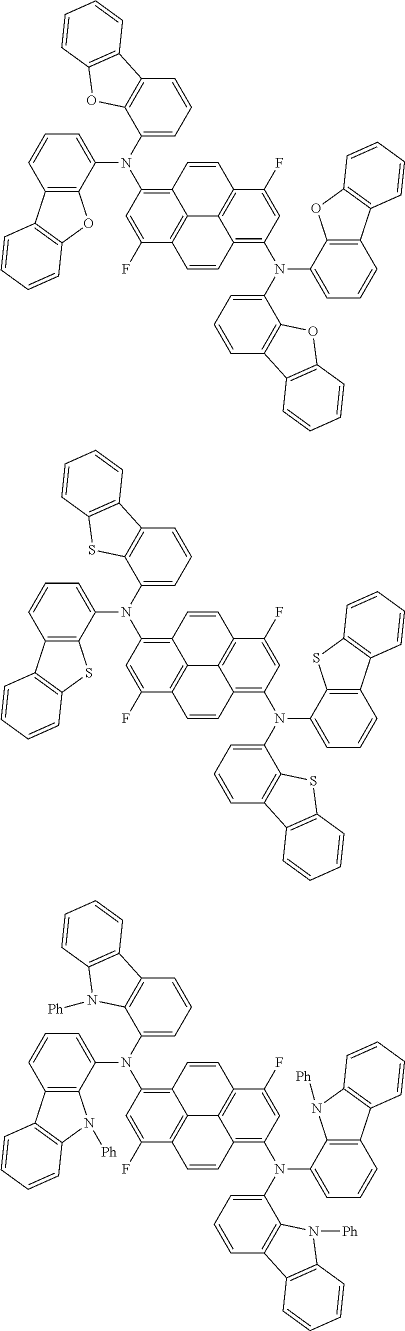 [SMILES: Fc1cc(N(c2cccc3c2oc2ccccc23)c2cccc3c2oc2ccccc23)c2ccc3c(F)cc(N(c4cccc5c4oc4ccccc45)c4cccc5c4oc4ccccc45)c4ccc1c2c34 ; Fc1cc(N(c2cccc3c2sc2ccccc23)c2cccc3c2sc2ccccc23)c2ccc3c(F)cc(N(c4cccc5c4sc4ccccc45)c4cccc5c4sc4ccccc45)c4ccc1c2c34 ; Fc1cc(N(c2cccc3c4ccccc4n(-c4ccccc4)c23)c2cccc3c4ccccc4n(-c4ccccc4)c23)c2ccc3c(F)cc(N(c4cccc5c6ccccc6n(-c6ccccc6)c45)c4cccc5c6ccccc6n(-c6ccccc6)c45)c4ccc1c2c34]

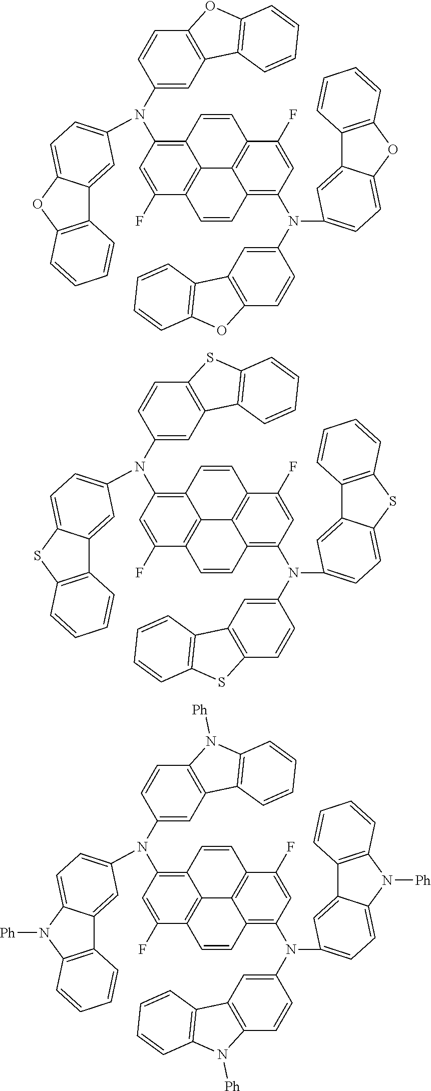

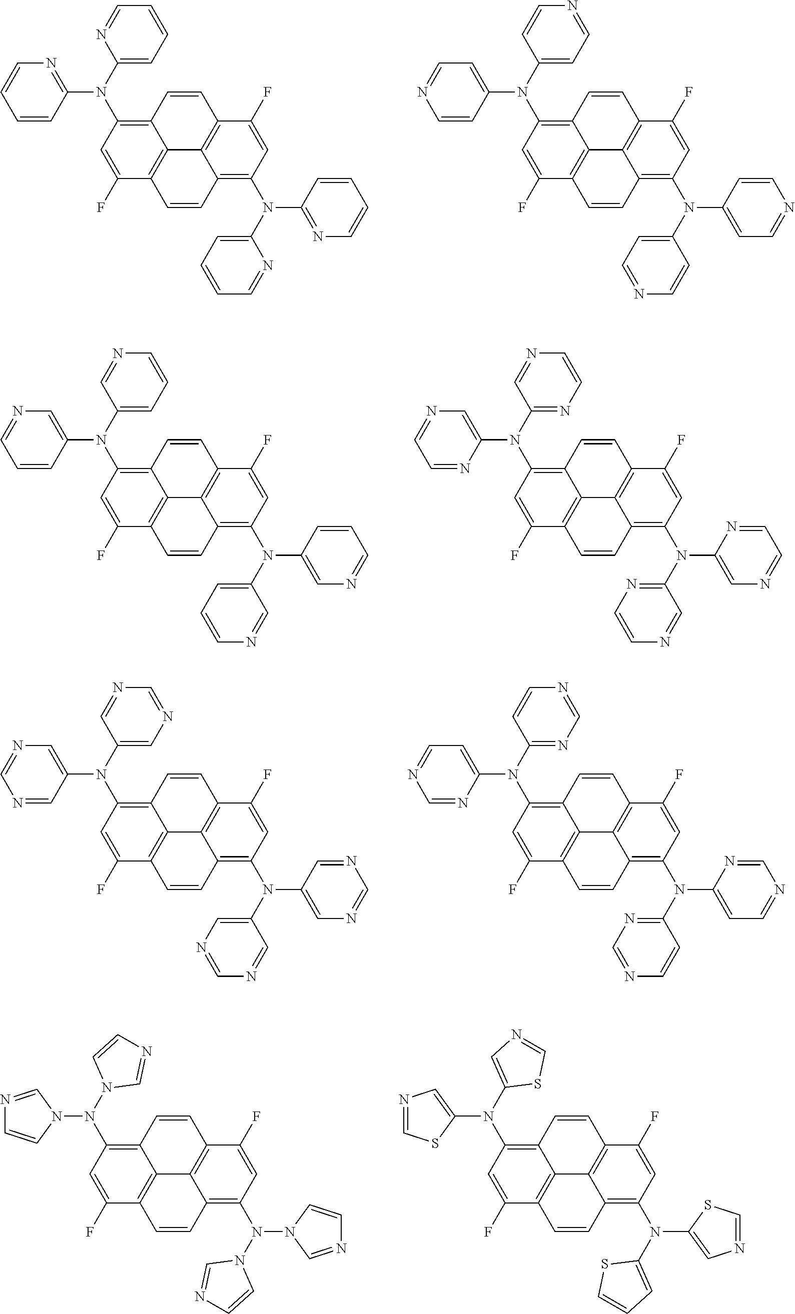

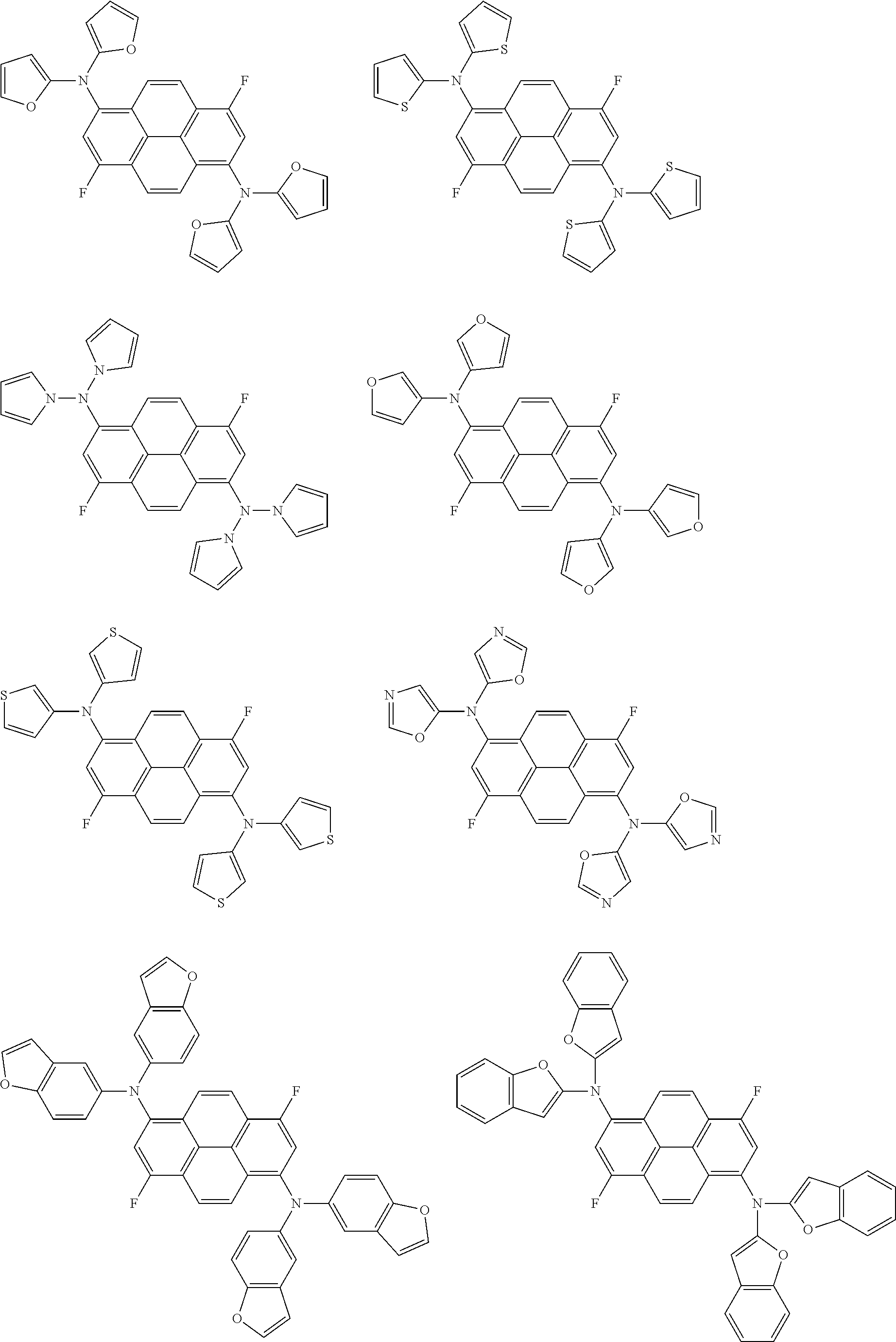

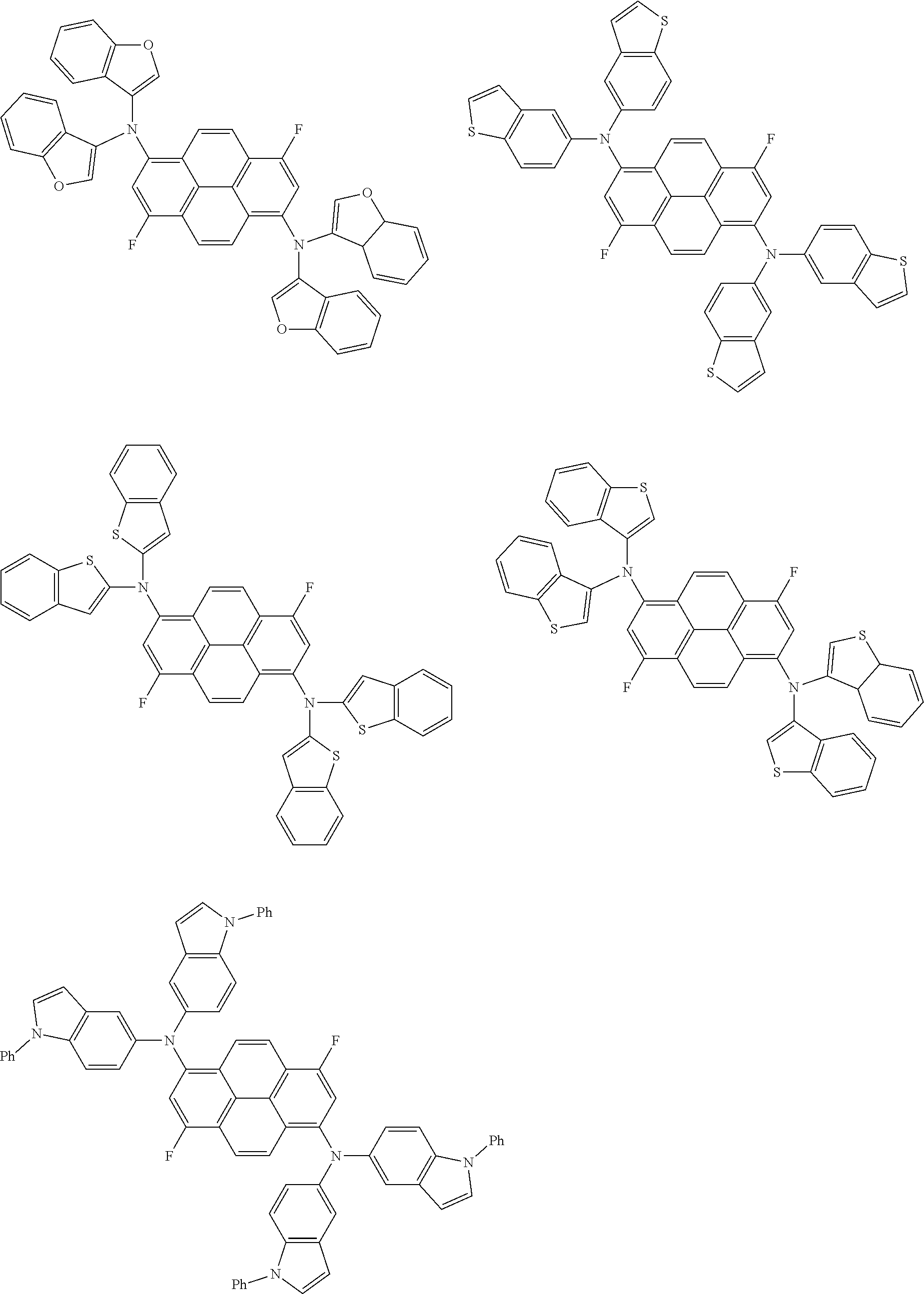

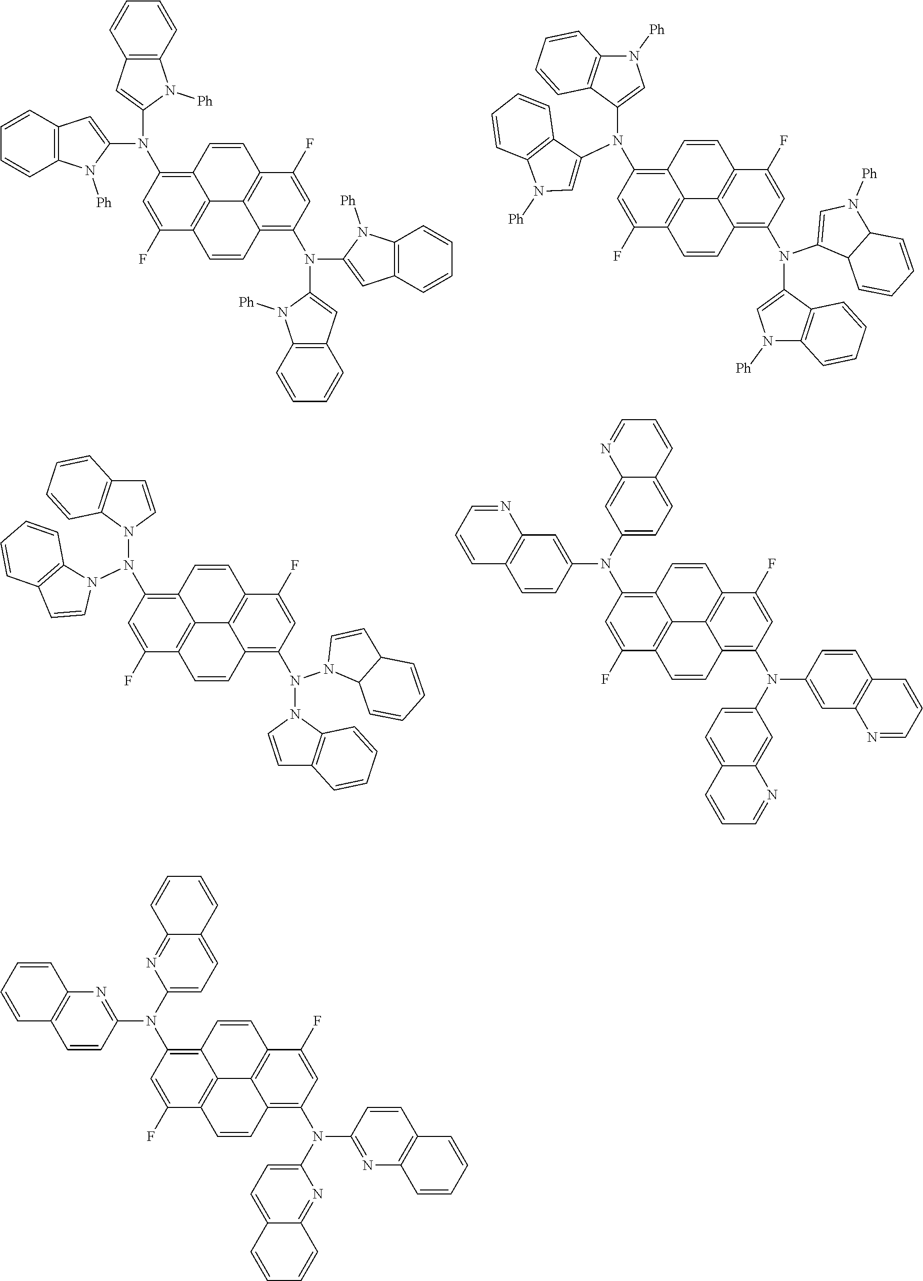

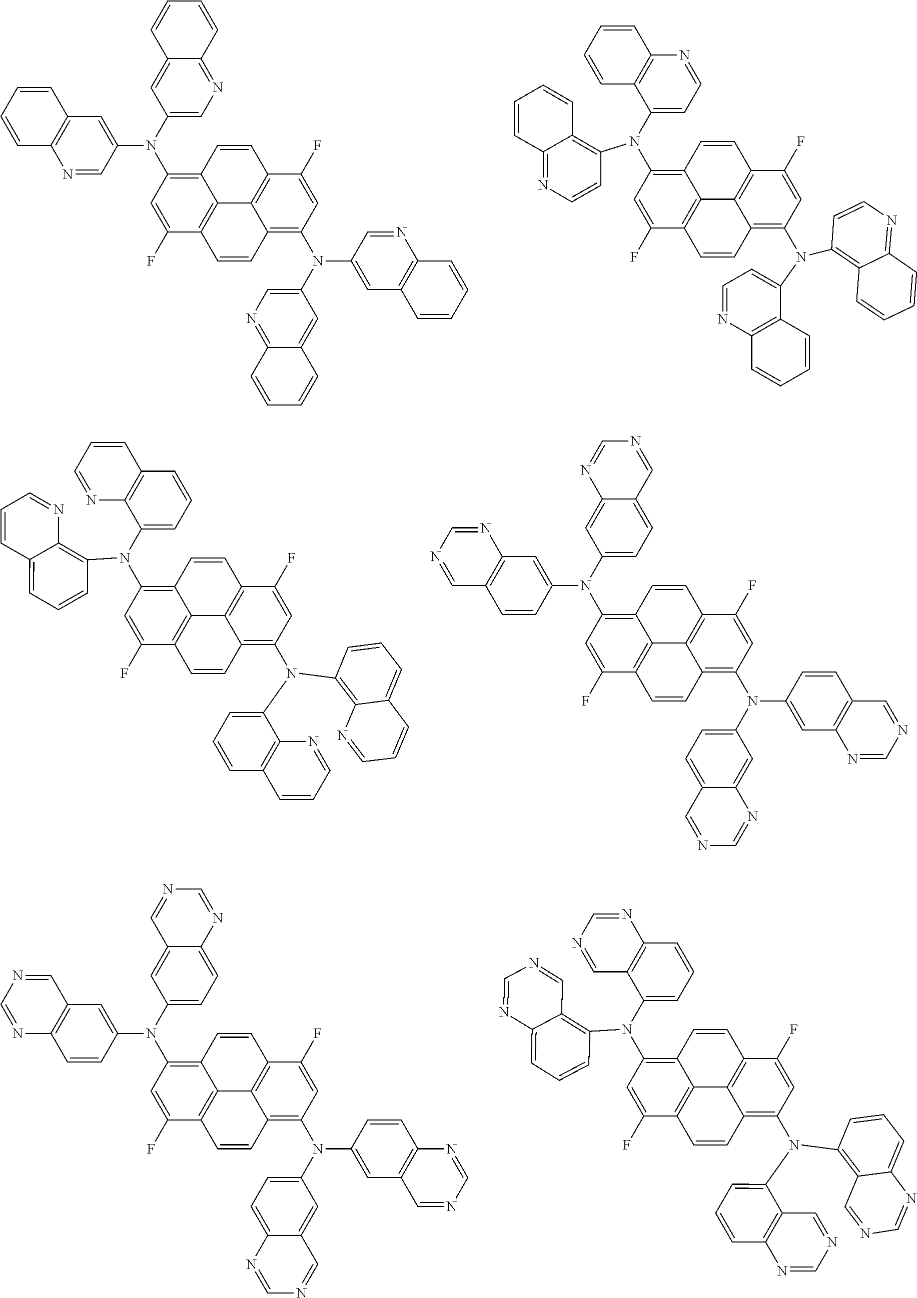

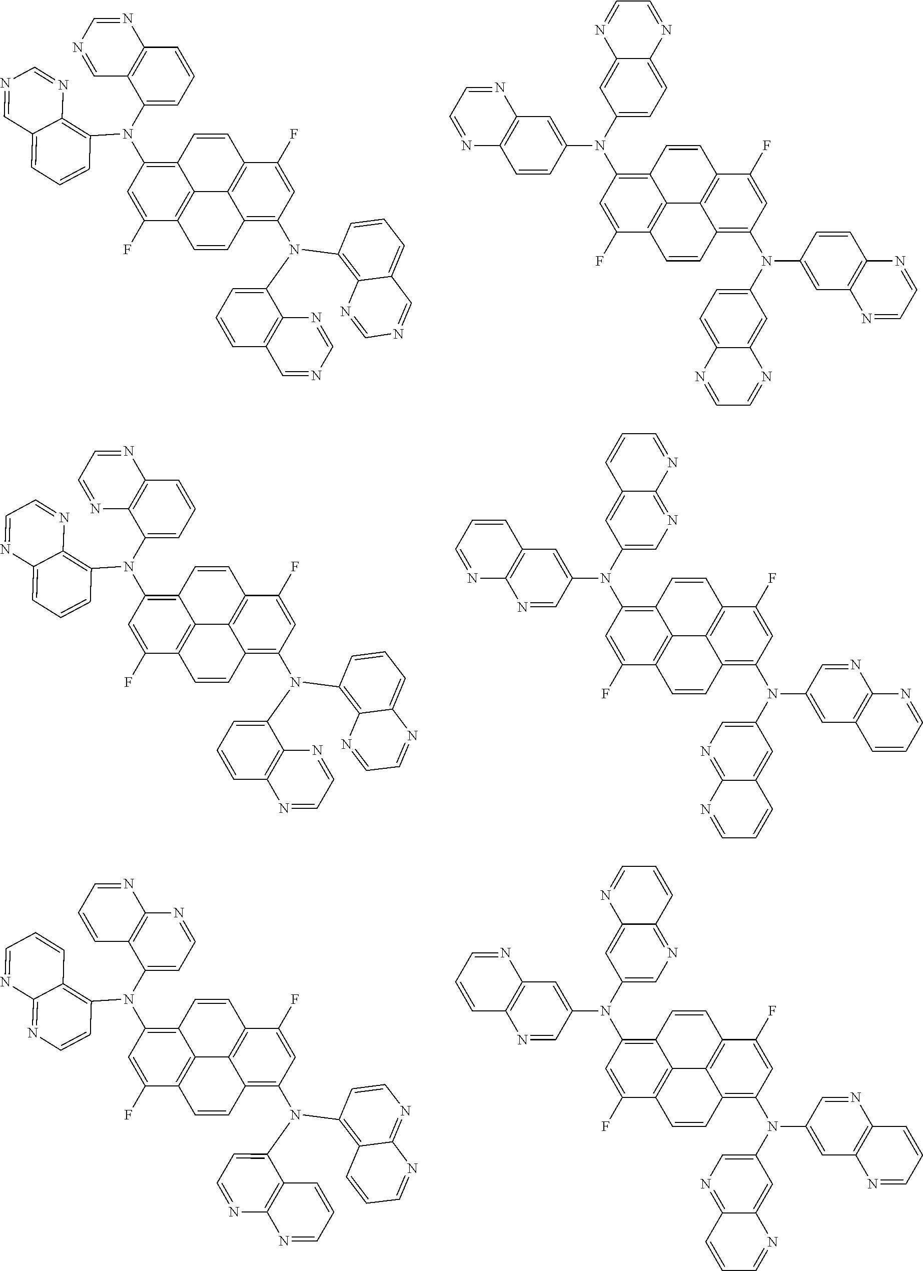

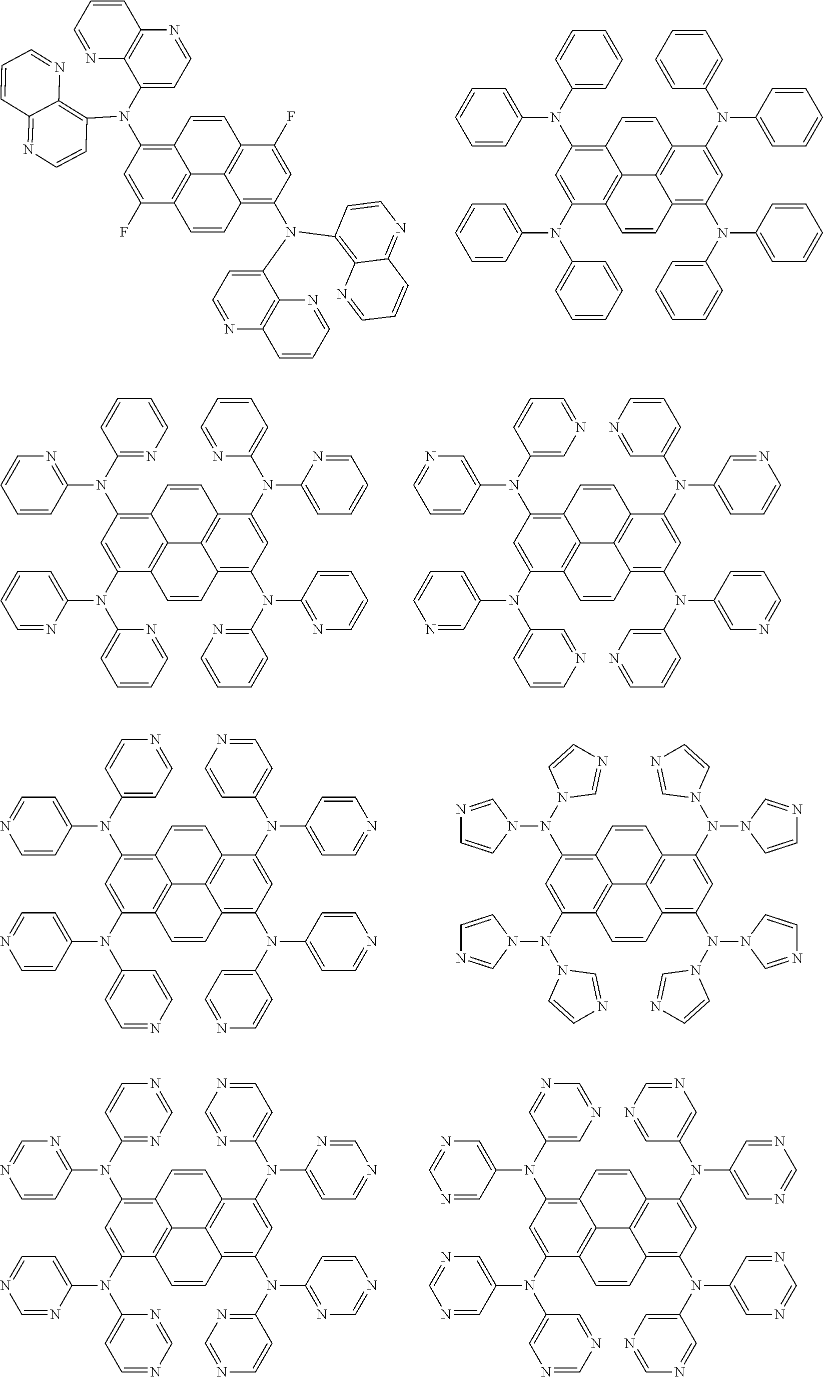

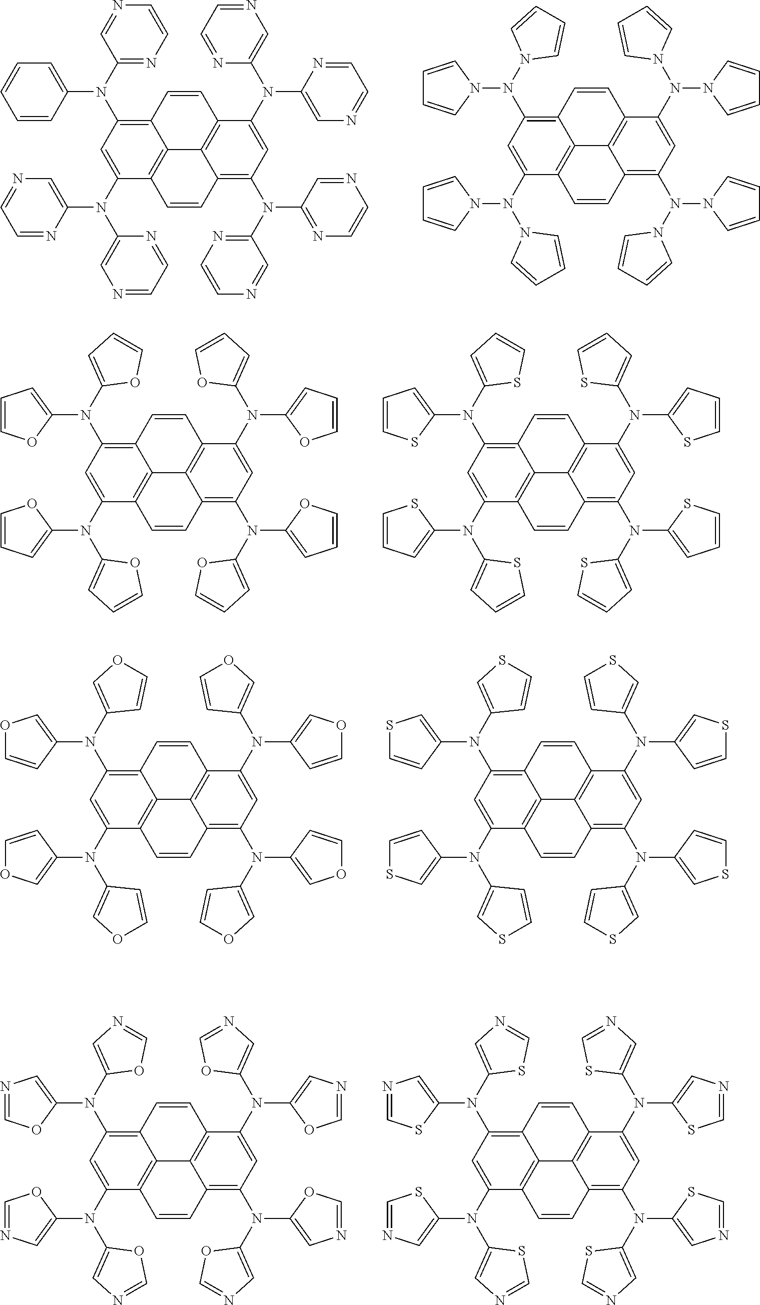

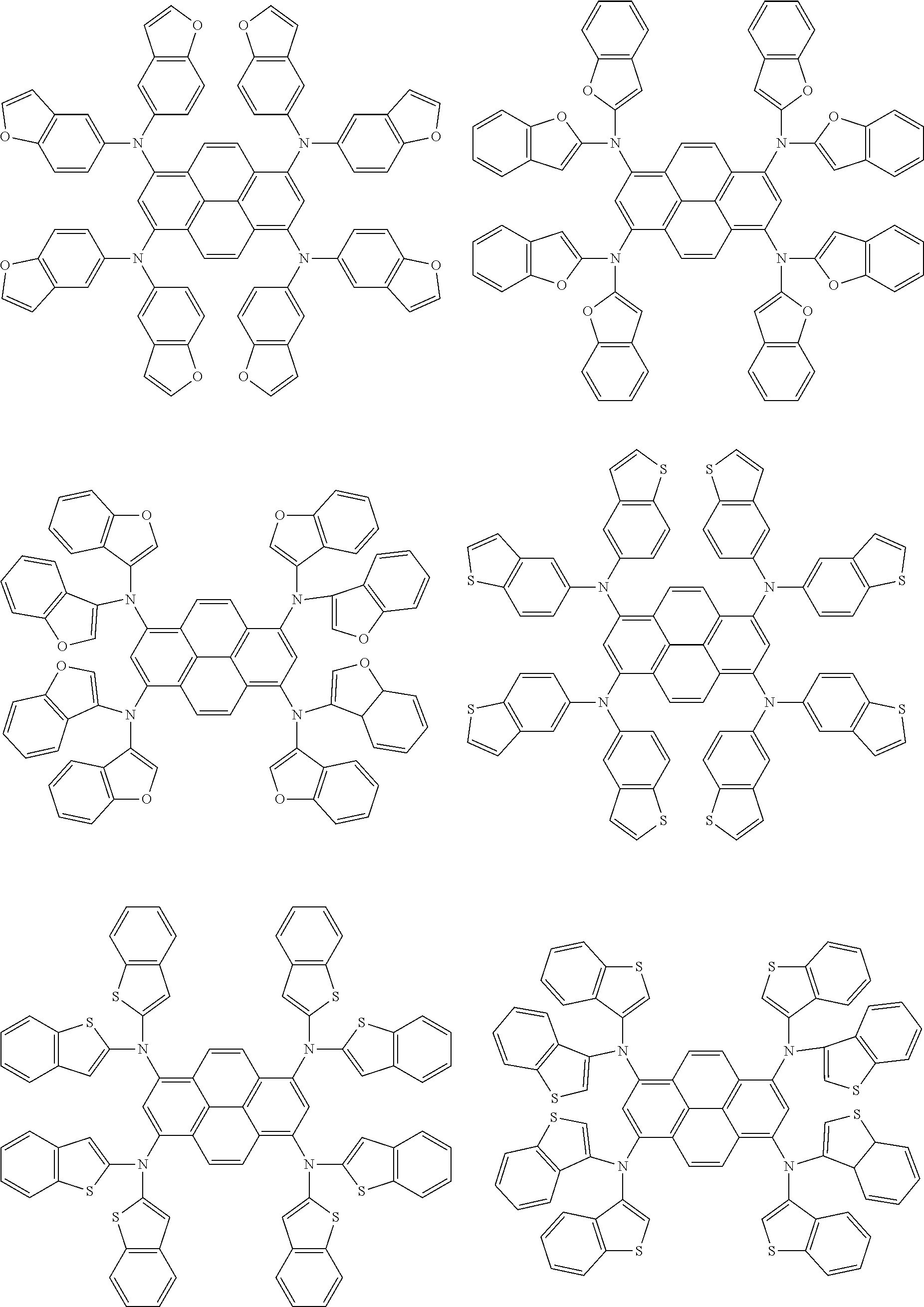

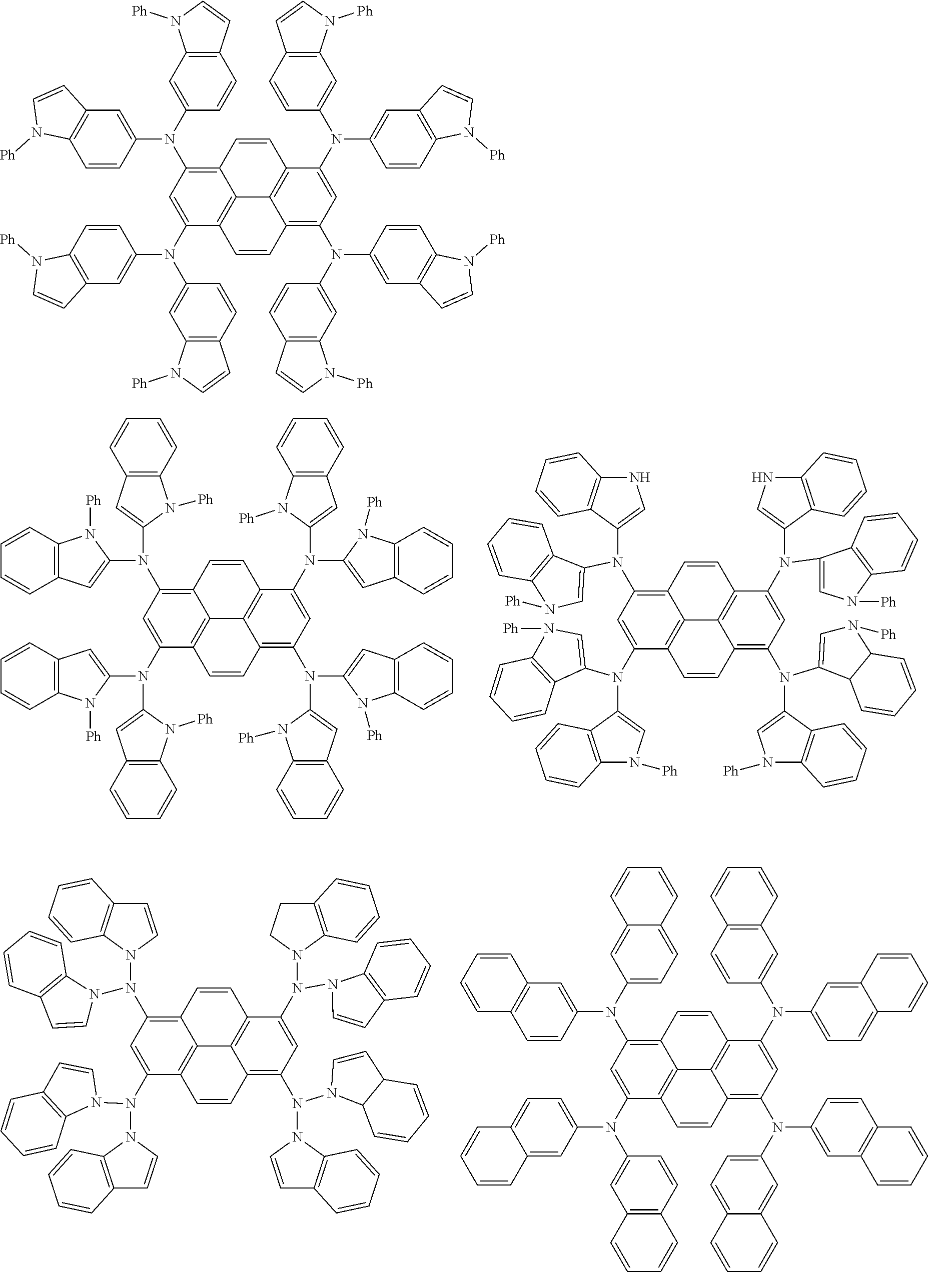

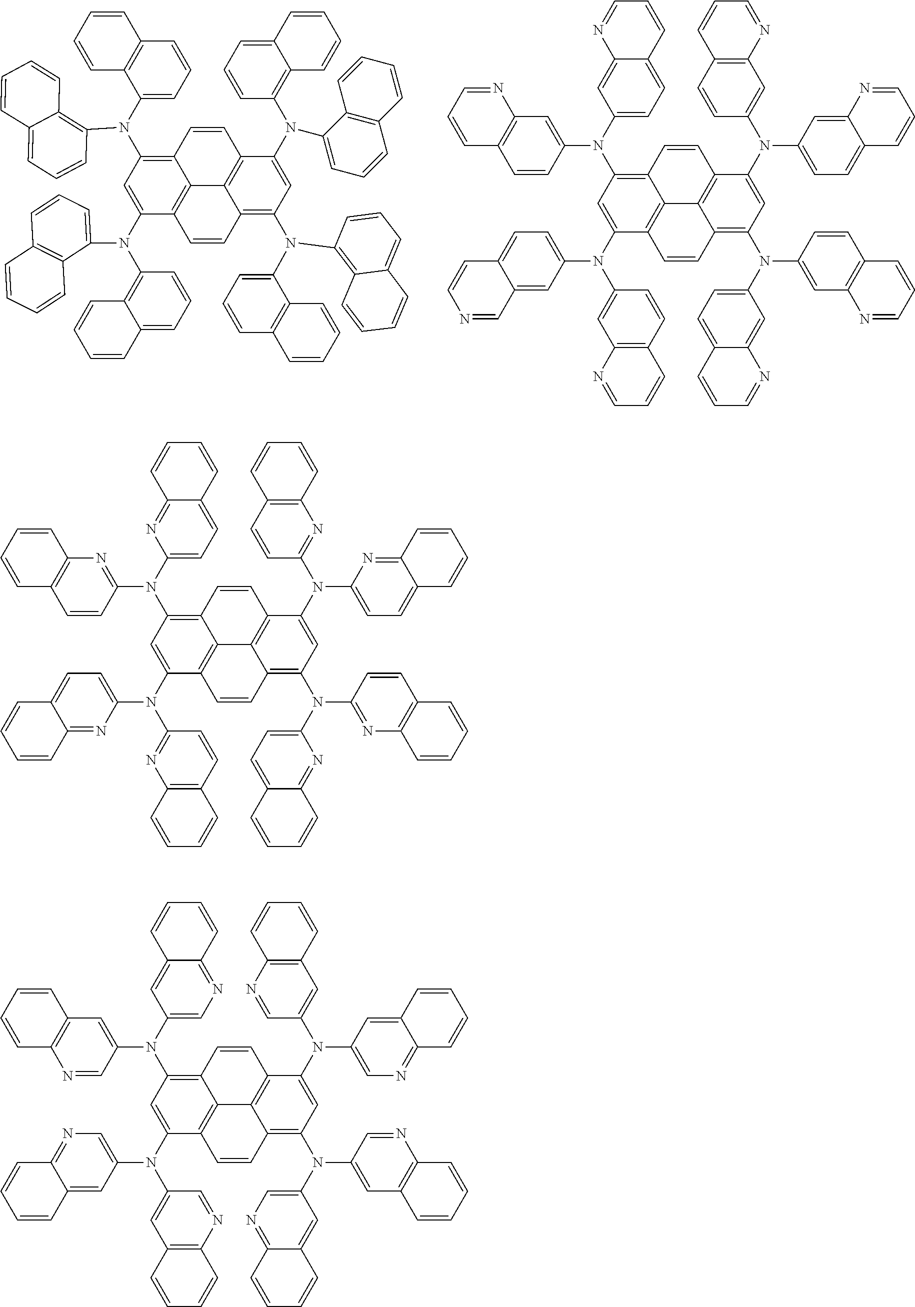

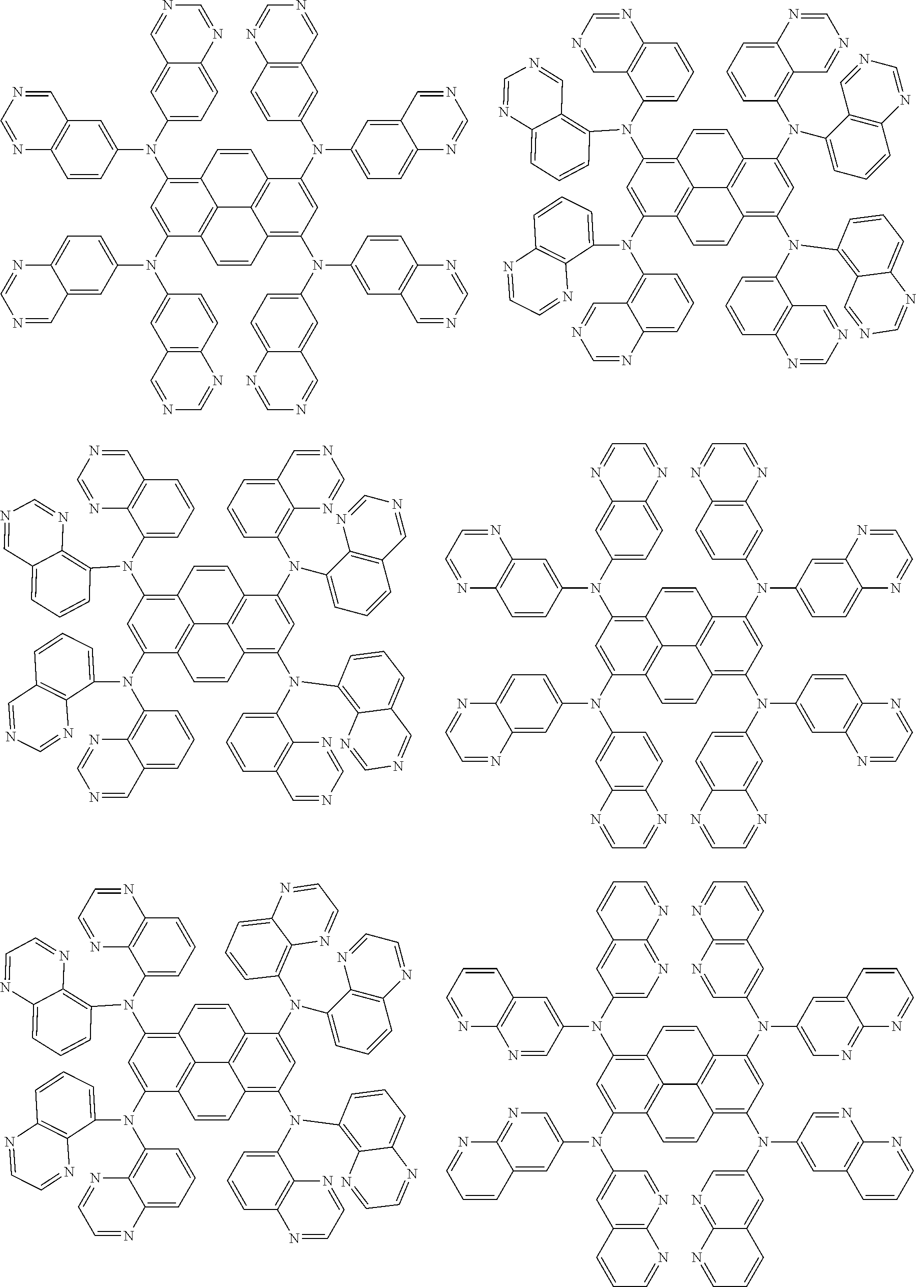

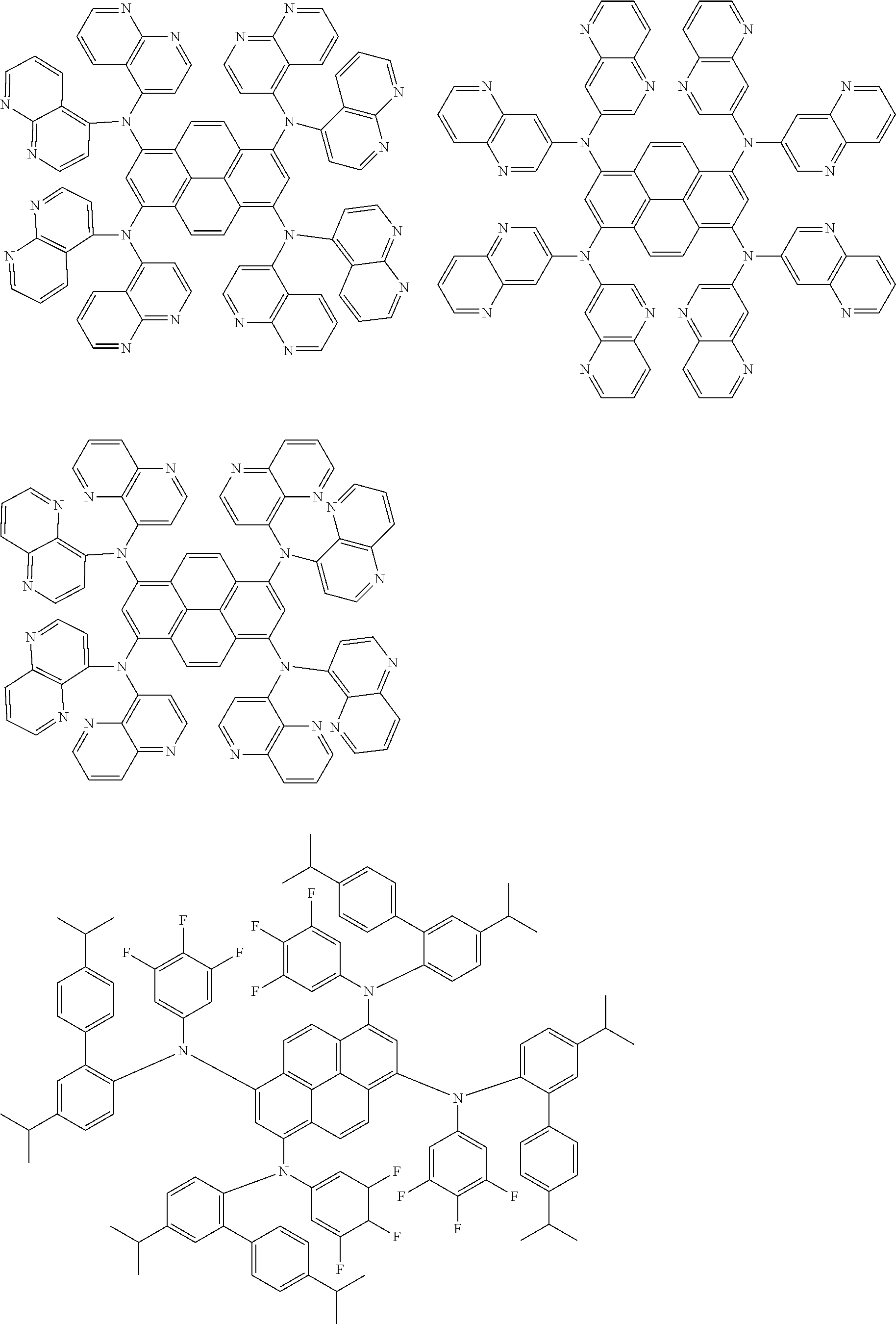

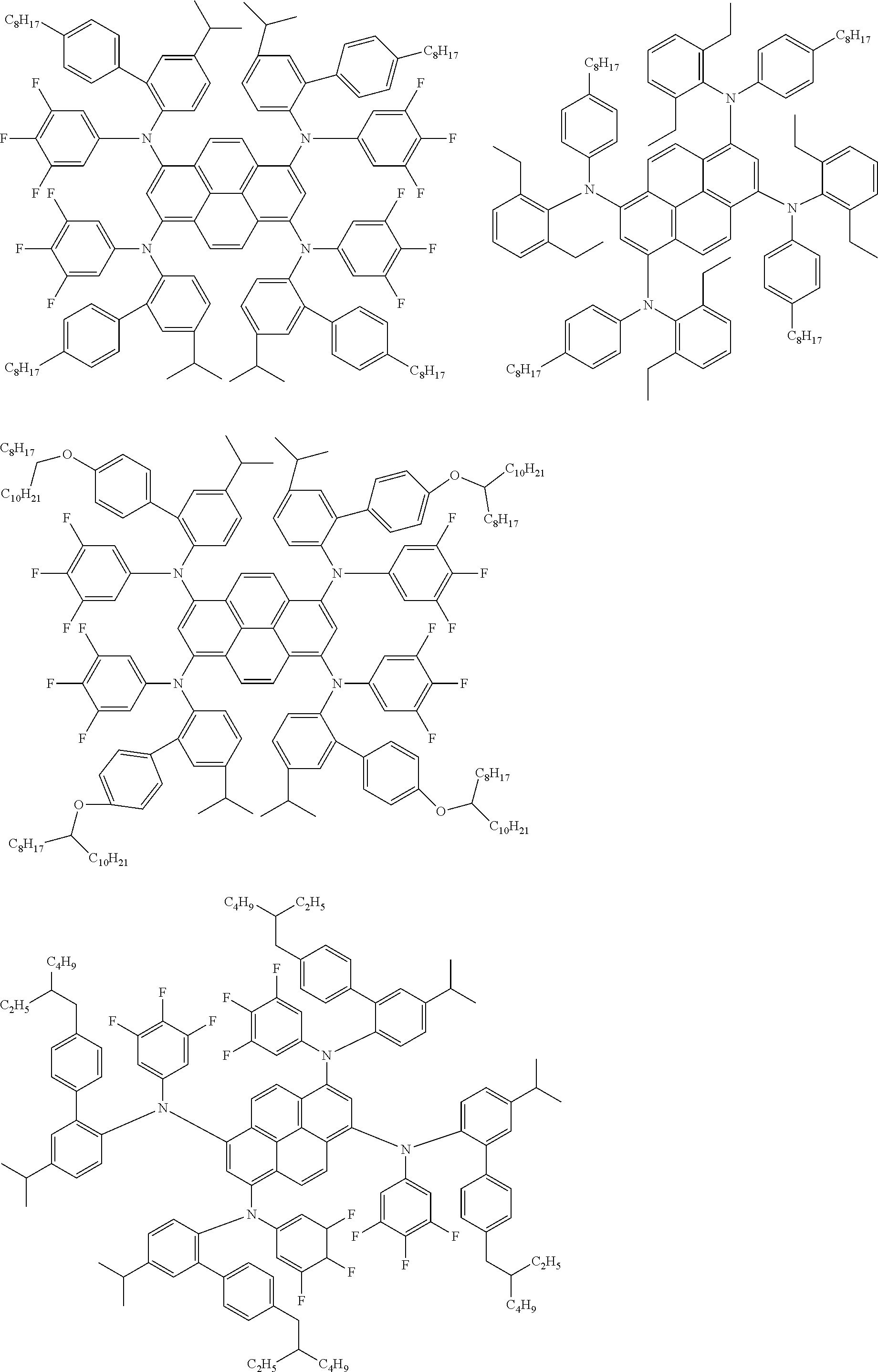

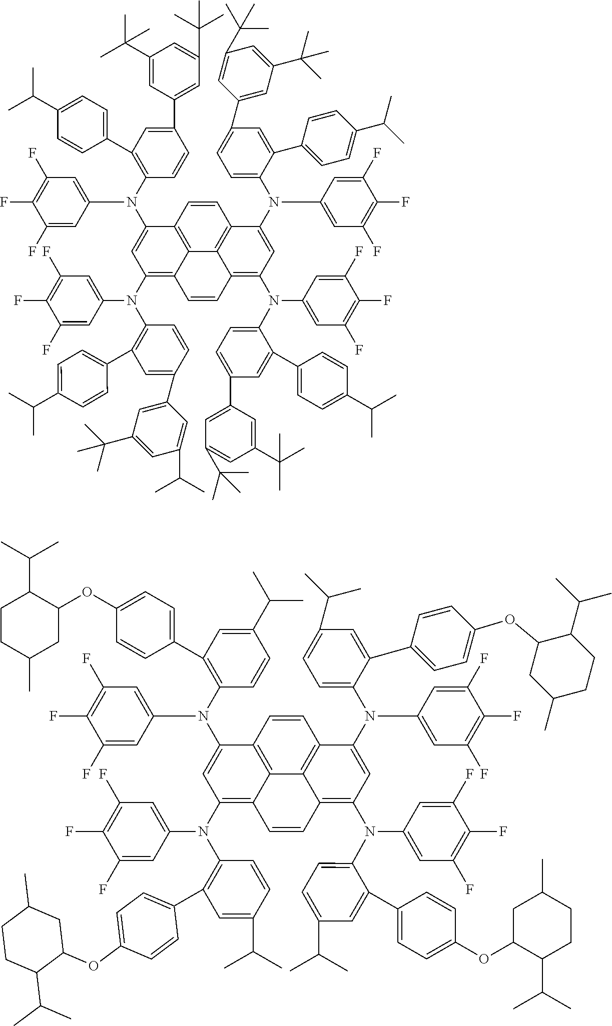

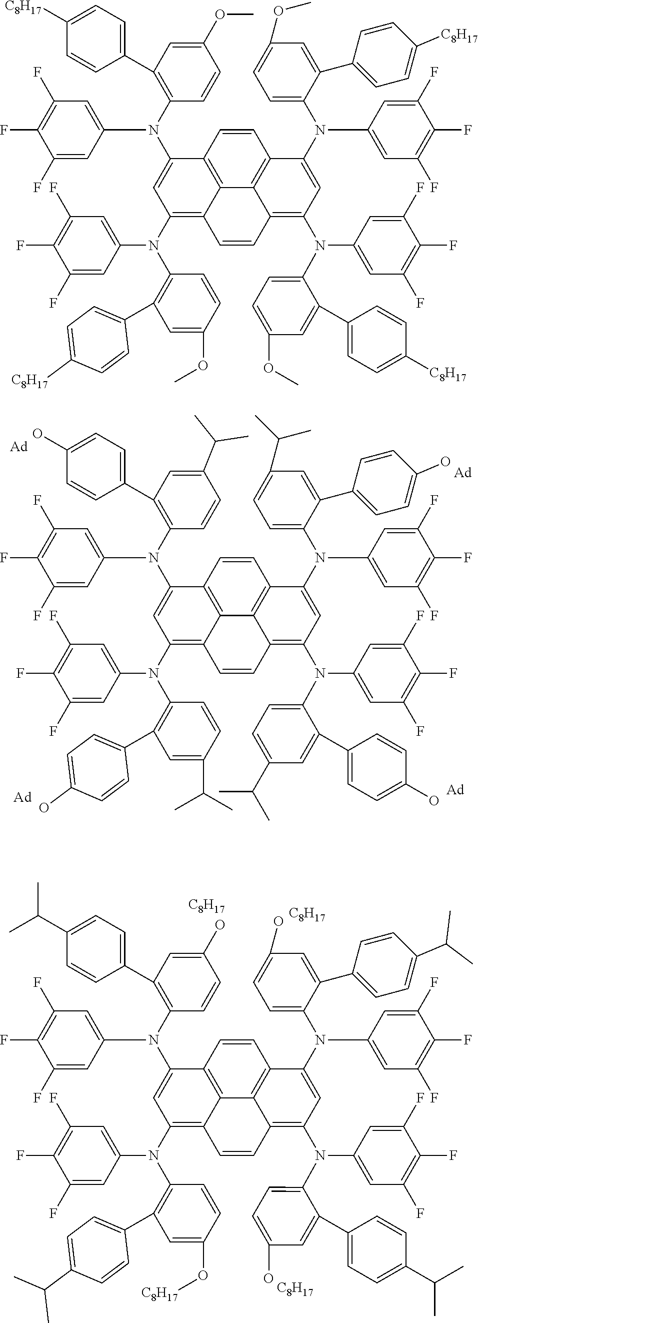

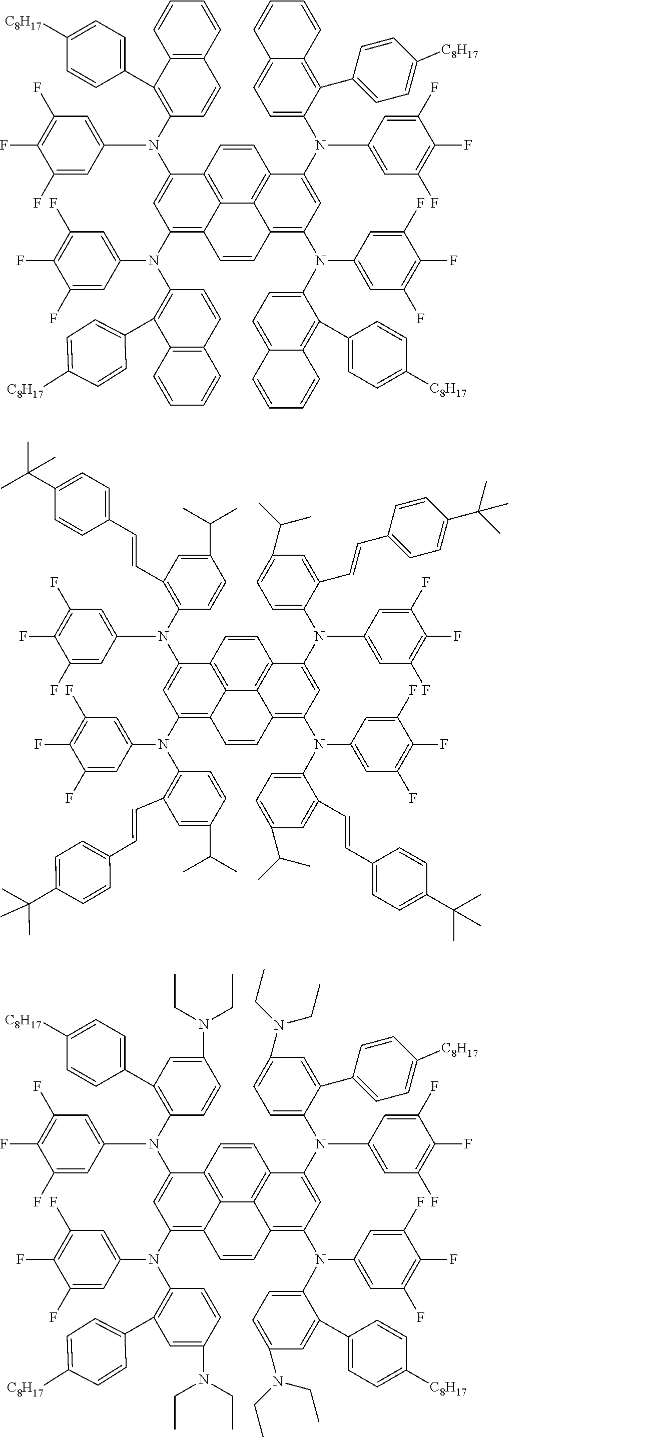

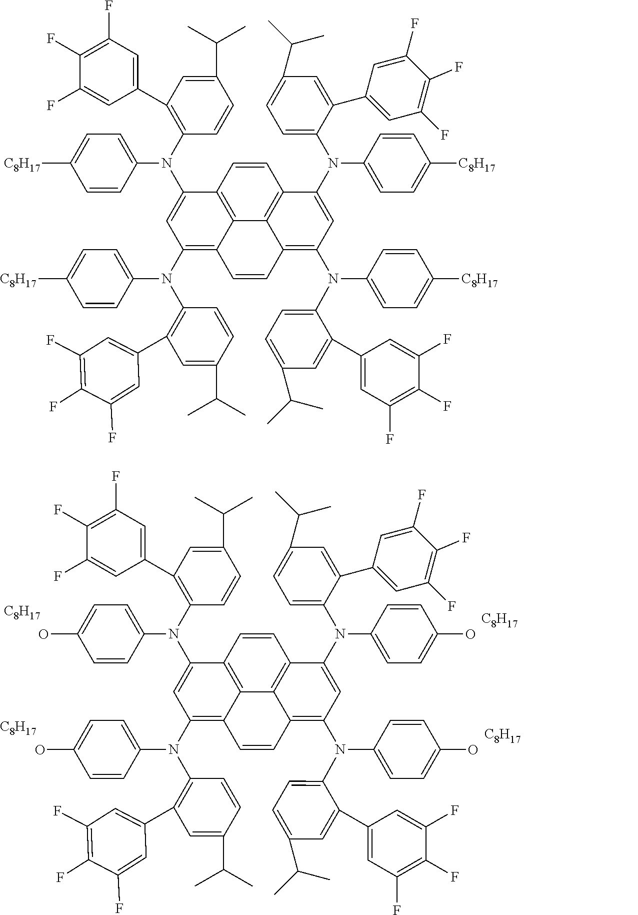

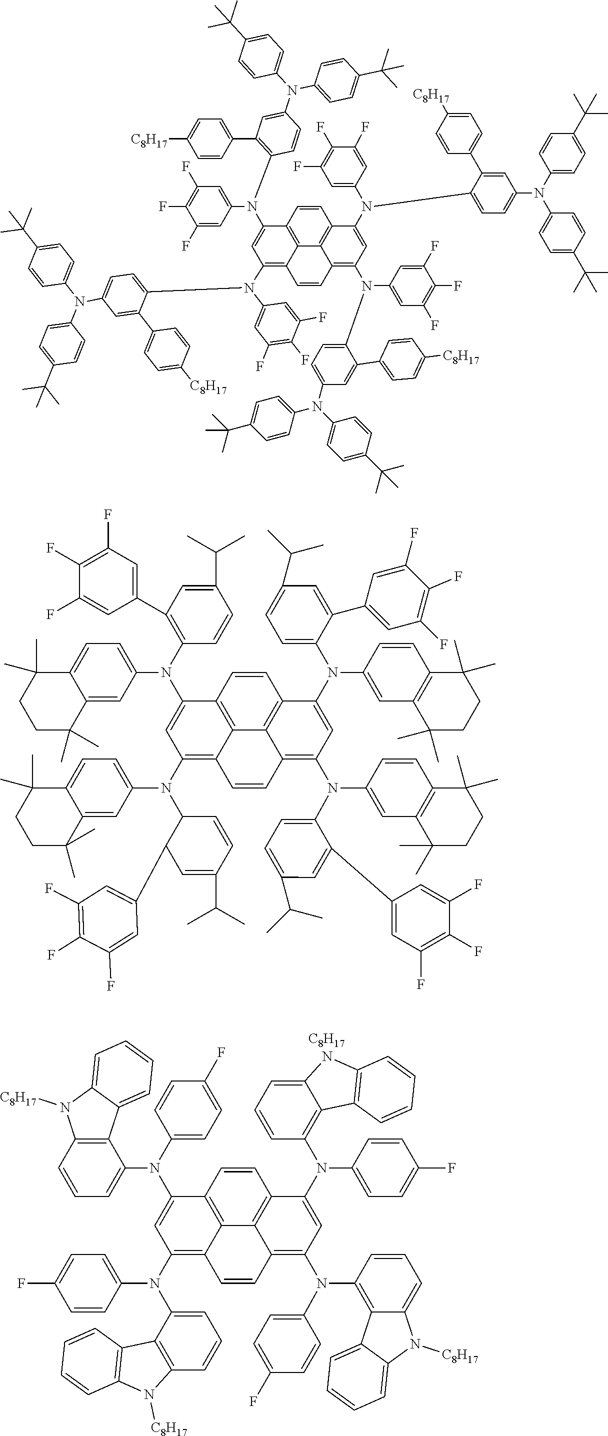

In the formulations as described herein, the absorption spectrum of the emitter E and the emission spectrum of the organic compound H have a large overlap, so that the efficient energy transfer (i.e., Forster resonance energy transfer (FRET)) can be realized therebetween.

In some embodiments, the emission spectrum of the formulation is derived exclusively from the emitter E, i.e. complete energy transfer is realized between the emitter E and the organic compound H.

In some embodiments, the formulation comprises more than two organic compounds H.

In some embodiments, the organic compound H is selected from one of formulas (1)-(1e) or (2)-(2e).

In some embodiments, in the formulation as described herein, the weight ratio of the organic compound H and the emitter E ranges from 50:50 to 99:1, preferably from 60:40 to 98:2, more preferably from 70:30 to 97:3, and most preferably from 80:20 to 95:5.

The present disclosure also relates to another formulation Z2 comprising an organic compound H2, an emitter D2, and an organic resin, where 1) the emission spectrum of the organic compound H2 is on the short wavelength side of the absorption spectrum of the emitter D2, and at least partially overlaps with the absorption spectrum of the emitter D2; 2) the emitter D2 comprises a structural unit of formula (3) or formula (4).

In some embodiments, the organic compound H2 of the another formulation Z2 is a compound comprising a structural unit represented by one of formulas (II-1)-(II-4),

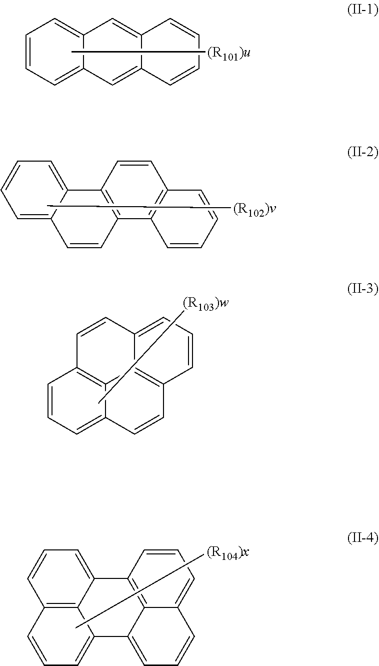

Where each of R₁₀₁ to R₁₀₄ is a substituent, and is independently selected from a C₁ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group (—C(═O)—H), an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents or any combination thereof.

u and w are natural numbers from 1 to 10, v and x are natural numbers from 1 to 12.

In some embodiments, the organic compound H2 comprises at least one alcohol-soluble or water-soluble group, as disclosed in International Application Publication No.

WO2022/213997A1; in some embodiments, the organic compound H2 comprises at least one cross-linkable group, as disclosed in International Application Publication No.

WO2022/213996A1. The patent documents listed above are specially incorporated herein by reference in their entirety.

In some embodiments, the emitter D2 of the another formulation Z2 is selected from formulas (3a), (4a), (3b), (4b), (3c), (4c), (3d), (4di), or (4d₂).

In some embodiments, the formulation or another formulation Z2 further comprises an organic resin and/or a solvent. For the purposes of the present disclosure, the organic resin refers to a resin prepolymer or a resin formed after the prepolymer is crosslinked or cured.

In some embodiments, the formulation or another formulation Z2 further comprises an organic resin. In some embodiments, the formulation comprises two and more organic resins.

The organic resins suitable for the present disclosure include, but not limited to: polystyrene, propylamine, polymethyl methacrylate, polycarbonate, polyurethane, polyvinylpyrrolidone, polyvinyl acetate, polybutylene, polyethylene glycol, silicone oil, epoxy resin, polyvinyl alcohol, polyacrylonitrile, polyvinylidene chloride (PVDC), polystyrene-acrylonitrile (SAN), polybutylene terephthalate (PBT), polyethylene terephthalate (PET), polyvinyl butyrate (PVB), polyvinyl chloride (PVC), polyamide, polyoxymethylene, polyimide, polyetherimide, and mixtures thereof.

Further, the organic resin suitable for the present disclosure includes, but not limited to, those prepared by the homopolymerization or copolymerization of the following monomers (resin prepolymers): styrene derivatives, acrylate derivatives, acrylonitrile derivatives, acrylamide derivatives, vinyl ester derivatives, vinyl ether derivatives, maleimide derivatives, conjugated diene derivatives.

Examples of styrene derivatives include, but not limited to, alkylstyrenes, such as α-methylstyrene, o-, m-, p-methylstyrene, p-butylstyrene; especially p-tert-butylstyrene, alkoxystyrene, such as p-methoxystyrene, p-butoxystyrene, p-tert-butoxystyrene.

Examples of acrylate derivatives include, but not limited to, methyl acrylate, methyl methacrylate, ethyl acrylate, ethyl methacrylate, n-propyl acrylate, n-propyl methacrylate, iso-propylacrylate, isopropyl methacrylate, n-butyl acrylate, n-butyl methacrylate, isobutyl acrylate, isobutyl methacrylate, sec-butyl acrylate, sec-butyl methacrylate, tert-butyl acrylate, tert-butyl methacrylate, 2-hydroxyethyl acrylate, 2-hydroxyethyl methacrylate, 2-hydroxypropyl acrylate, 2-hydroxypropyl methacrylate, 3-hydroxypropyl acrylate, 3-hydroxypropyl methacrylate, 2-hydroxybutyl acrylate, 2-hydroxybutyl methacrylate, 3-hydroxybutyl acrylate, 3-hydroxybutyl methacrylate, 4-hydroxybutyl acrylate, 4-hydroxybutyl methacrylate, allyl acrylate, allyl methacrylate, benzyl acrylate, benzyl methacrylate, cyclohexyl acrylate, cyclohexyl methacrylate, phenyl acrylate, phenyl methacrylate, 2-methoxyethyl acrylate, 2-methoxyethyl methacrylate, 2-phenoxyethyl acrylate, 2-phenoxyethyl methacrylate, methoxydiethylene glycol acrylate, methoxydiethylene glycol methacrylate, methoxytriethylene glycol acrylate, methoxytriethylene glycol methacrylate, methoxypropylene glycol acrylate, methoxypropylene glycol methacrylate, methoxy dipropylene glycol acrylate, methoxydipropylene glycol methacrylate, isobornyl acrylate, isobornyl methacrylate, dihydrodicyclopentadienyl acrylate, dicyclopentadiene methacrylate, adamantane (meth) acrylate, norbornene (meth) acrylate, 2-hydroxy-3-phenoxypropyl acrylate, 2-hydroxy-3-phenoxypropyl methacrylate, glycerol monoacrylate, glycerol monomethacrylate, 2-aminoethyl acrylate, 2-aminoethyl methacrylate, 2-dimethylaminoethyl acrylate, 2-dimethylaminoethyl methacrylate, N,N-dimethylaminoethyl (meth) acrylic acid, N,N-diethylaminoethyl (meth) acrylate, 2-aminopropyl acrylate, 2-aminopropyl methacrylate, 2-dimethylaminopropyl acrylate, 2-dimethylaminopropyl methacrylate, 3-aminopropyl acrylate, 3-aminopropyl methacrylate, benzyl N,N-dimethyl-1,3-propanediamine(meth)acrylate, 3-dimethylaminopropyl acrylate, 3-dimethylaminopropyl methacrylate, glycidyl acrylate, and glycidyl methacrylate.

Examples of acrylonitrile derivatives include, but not limited to, acrylonitrile, methacrylonitrile, 2-chloroacrylonitrile, and vinylidene cyanide.

Examples of acrylamide derivatives include, but not limited to, acrylamide, methacrylamide, a-chloroacrylamide, N-2-hydroxyethyl acrylamide, and N-2-hydroxyethyl methacrylamide.

Examples of vinyl ester derivatives include, but not limited to vinyl acetate, vinyl propionate, vinyl butyrate and vinyl benzoate.

Examples of vinyl ether derivatives include, but not limited to vinyl methyl ether, vinyl ethyl ether and allyl glycidyl ether.

Examples of maleimide derivatives include, but not limited to maleimide, benzylmaleimide, N-phenylmaleimide and N-cyclohexylmaleimide.

Examples of conjugated diene derivatives include, but not limited to 1,3-butadiene, isoprene and chloroprene.

The homopolymers or copolymers can be prepared by free radical polymerization, cationic polymerization, anionic polymerization, or organometallic catalytic polymerization (for example Ziegler-Natta catalysis). The process of polymerization can be suspension polymerization, emulsion polymerization, solution polymerization, or bulk polymerization.

The number average molecular weight Mn (as determined by GPC) of the organic resins is generally in the range of 10 000 g/mol to 1 000 000 g/mol, preferably in the range of 20 000 g/mol to 750 000 g/mol, more preferably in the range of 30 000 g/mol to 500 000 g/mol.

In some embodiments, the organic resin is a thermosetting resin or an UV curable resin.

In some embodiments, the organic resin is cured by a method that will enable roll-to-roll processing.

Thermosetting resins require curing in which they undergo an irreversible process of molecular cross-linking, which makes the resin non-fusible. In some embodiments, the thermosetting resin is an epoxy resin, a phenolic resin, a vinyl ester resin, a melamine co-polycondensation resin, an urea-formaldehyde resin, an unsaturated polyester resin, a polyurethane resin, an allyl resin, an acrylic resin, a polyamide resin, a polyamide-imide resin, a phenol-amide polycondensation resin, an urea-melamine polycondensation resin, or any combination thereof.

In some embodiments, the thermosetting resin is an epoxy resin. The epoxy resins are easy to cure and do not give off volatiles or generate by-products from a wide range of chemicals. The epoxy resins can also be compatible with most substrates and tend to readily wet surfaces. See also Boyle, M. A. et al., “Epoxy Resins”, Composites, Vol. 21, ASM Handbook, pages 78-89 (2001).

In some embodiments, the organic resin is a silicone thermosetting resin. In some embodiments, the silicone thermosetting resin is OE6630A or OE6630B (Dow Corning Corporation (Auburn, Michigan.)).

In some embodiments, a thermal initiator is used. In some embodiments, the thermal initiator is AIBN[2,2′-azobis(2-methylpropionitrile)] or benzoyl peroxide.

The UV curable resin is a polymer that will cure and rapidly harden upon exposure to light of a specific wavelength. In some embodiments, the UV curable resin is a resin having a free radical polymerization group, and a cationic polymerizable group as functional groups; the radical polymerizable group is such as (meth)acryloyloxy group, vinyloxy group, styryl group, or vinyl group. The cationically polymerizable group is, for example, epoxy group, thioepoxy group, vinyloxy group, or oxetanyl group. In some embodiments, the UV curable resin is a polyester resin, a polyether resin, a (meth)acrylic resin, an epoxy resin, a polyurethane resin, an alkyd resin, a spiroacetal resin, a polybutadiene resin, or a thiolene resin.

In some embodiments, the UV curable resin is selected from polyurethane acrylate, allyloxy diacrylate, bis(acryloyloxyethyl) hydroxyisocyanurate, bis(acryloyloxyneopentyl glycol) adipate, bisphenol A diacrylate, bisphenol A dimethacrylate, 1,4-butanediol diacrylate, 1,4-butanediol dimethacrylate, 1,3-butanediol diacrylate, 1,3-butanediol dimethacrylate, dicyclopentyl diacrylate, diethylene glycol diacrylate, diethylene glycol dimethacrylate, dipentaerythritol hexaacrylate, dipentaerythritol monohydroxy pentacrylate, bis(trimethylolpropane) tetraacrylate, triethylene glycol dimethacrylate, glyceryl methacrylate, 1,6-hexanediol diacrylate, neopentyl glycol dimethacrylate, neopentyl glycol hydroxypivalonate, pentaerythritol triacrylate, pentaerythritol tetraacrylate, dimethacrylate, polyethylene glycol diacrylate, polypropylene glycol diacrylate, tetraethylene glycol diacrylate, tetrabromobisphenol A diacrylate, triethylene glycol divinyl ether, glycerol diacrylate, trimethylolpropane triacrylate, tripropylene glycol diacrylate, tris(acryloyloxyethyl) isocyanurate, triacrylate, diacrylate, propyl acrylate, vinyl-terminated polydimethylsiloxane, vinyl-terminated diphenyl siloxane-dimethyl siloxane copolymer, vinyl-terminated polyphenyl methyl siloxane, vinyl-terminated difluoromethyl siloxane-dimethyl siloxane copolymer, vinyl-terminated diethyl siloxane-dimethyl siloxane copolymer, vinyl methyl siloxane, monomethacryloxypropyl-terminated polydimethylsiloxane, monovinyl-terminated polydimethylsiloxane, monoallyl-mono-trimethylsilyloxy-terminated polyethylene oxide, or any combination thereof.

In some embodiments, the UV curable resin is a mercapto functional compound that can be cross-linked under UV curing conditions with an isocyanate, an epoxy resin, or an unsaturated compound. In some embodiments, the mercapto functional compound is a polythiol. In some embodiments, the polythiol is selected from: pentaerythritol tetrakis(3-mercaptopropionate) (PETMP), trimethylolpropane tris(3-mercaptopropionate) (TMPMP), ethylene glycol bis(3-mercaptopropionate) (GDMP); tris[25-(3-mercapto-propionyloxy)ethyl]isocyanurate (TEMPIC), dipentaerythritol hexa(3-mercaptopropionate) (Di-PETMP), ethoxylated trimethylolpropane tri(3-mercaptopropionate) (ETMP1300 and ETTMP700), polycaprolactone tetra(3-mercaptopropionate) (PCL4MP1350), pentaerythritol tetramercaptoacetate (PETMA), trimethylolpropane trimercaptoacetate (TMPMA), or ethylene glycol dimercaptoacetate (GDMA). These compounds are sold under the trade name THIOCURE® by Bruno Bock (Malsacht, Germany).

In some embodiments, the UV curable resin further comprises photoinitiator. The photoinitiator will initiate crosslinking and/or curing reactions of the photosensitive material during exposure to light. In some embodiments, the photoinitiator is a compound such as acetophenone-based, benzoin-based, or thidrone-based that initiate the polymerization, crosslinking and curing of monomers.

In some embodiments, the UV curable resin comprises mercapto-functional compound, methacrylate, acrylate, isocyanate, or combinations thereof. In some embodiments, the UV curable resin comprises polythiols, methacrylates, acrylates, isocyanates, or any combination thereof.

In some embodiments, the photoinitiator is MINS-311RM (Minuta Technology Co., Ltd (Korea)).

In some embodiments, the photoinitiator is Irgacure® 127, Irgacure® 184, Irgacure® 184D, Irgacure® 2022, Irgacure® 2100, Irgacure® 250, Irgacure® 270, Irgacure® 2959, Irgacure® 369, Irgacure® 369EG, Irgacure® 379, Irgacure® 500, Irgacure® 651, Irgacure® 754, Irgacure® 784, Irgacure® 819, Irgacure® 819DW, Irgacure® 907, Irgacure® 907FF, Irgacure® OxeOl, Irgacure® TPO-L, Irgacure® 1173, Irgacure® 1173D, Irgacure® 4265, Irgacure® BP, or Irgacure® MBF (BASF Corporation (Wyandotte, Michigan)). In some embodiments, the photoinitiator is TPO (2,4,6-trimethylbenzoyl-diphenyl-oxide) or MBF (methyl benzoyl formate).

In some embodiments, the weight percentage of organic resin in the formulation is about 20% to about 99%, about 20% to about 95%, about 20% to about 90%, about 20% to about 85%, about 20% to about 80%, about 20% to about 70%, about 20% to about 60%, about 40% to about 99%, about 40% to about 95%, about 40% to about 90%, about 40% to about 85%, about 40% to about 80%, about 40% to about 70%, about 70% to about 99%, about 70% to about 95%, about 70% to about 90%, about 70% to about 85%, about 70% to about 80%, about 80% to about 99%, about 80% to about 95%, about 80% to about 90%, about 80% to about 85%, about 85% to about 99%, about 85% to about 95%, about 85% to about 90%, about 90% to about 99%, about 90% to about 95%, or about 95% to about 99%.

In another aspect, the present disclosure also provides a formulation or another formulation Z2 comprising at least one solvent. In some embodiments, the formulation as described herein is a solution.

In some embodiments, the formulation or another formulation Z2 as described herein is a dispersion.

The formulation in the embodiments as described herein may comprise the emitter E of 0.01 wt % to 20 wt %, preferably 0.1 wt % to 20 wt %, more preferably 0.2 wt % to 20 wt %, and most preferably 2 wt % to 15 wt %.

Another formulation Z2 in the embodiments as described herein may comprise the emitter D2 of 0.01 wt % to 20 wt %, preferably 0.1 wt % to 20 wt %, more preferably 0.2 wt % to 20 wt %, and most preferably 2 wt % to 15 wt %.

Using the formulation or another formulation Z2 as described herein, the color conversion layer may be fabricated by ink-jet printing, transfer printing, photolithography, etc. In this case, the organic compound H (i.e., the color conversion material) needs to be dissolved alone or together with other materials in a resin (prepolymer) and/or an organic solvent, to form a ink. The mass concentration of the organic compound H (i.e., the color conversion material) in the ink is not less than 0.1 wt %. The color conversion ability of the color conversion layer can be tuned by adjusting the concentration of the color conversion material in the ink and the thickness of the color conversion layer. In general, the higher the concentration of the color conversion material or the thickness of the layer, the higher the color conversion efficiency of the color conversion layer would be.

In some embodiments, the solvent is selected from: water, alcohol, ester, aromatic ketone, aromatic ether, aliphatic ketone, aliphatic ether, inorganic ester compounds such as boronic ester or phosphoric ester, or a combination of two or more of them.

In some embodiments, the suitable and preferred solvents include, but not limited to, aliphatics, alicyclics, aromatics, amines, thiols, amides, nitriles, esters, ethers, polyethers, alcohols, diols, or polyols.

In some embodiments, the alcohol represents a solvent of the suitable class. The preferred alcohol includes alkylcyclohexanol, especially methylated aliphatic alcohol, naphthol, etc.

Examples of the suitable alcohol solvents include, but not limited to, dodecanol, phenyltridecanol, benzyl alcohol, ethylene glycol, 2-methoxyethanol, glycerol, propylene glycol, propyleneglycol monoethyl ether, etc.

The solvent may be used alone or as a combination of two or more organic solvents.

Further, examples of organic solvents, include (but are not limited to): methanol, ethanol, 2-methoxyethanol, dichloromethane, trichloromethane, chlorobenzene, 1,2-dichlorobenzene, tetrahydrofuran, anisole, morpholine, toluene, o-xylene, m-xylene, p-xylene, 1,4-dioxane, acetone, 2-butanone, 1,2-dichloroethane, 3-phenoxytoluene, 1,1,1-trichloroethane, 1,1,2,2-tetrachloroethane, ethyl acetate, butyl acetate, N,N-dimethylformamide, N,N-dimethylacetamide, dimethyl sulfoxide, tetraline, decahydronaphthalene, indene, and/or any combination thereof.

In some embodiments, in the formulation or another formulation Z2 as described herein, the solvent is selected from aromatic, heteroaromatic, ester, aromatic ketone, aromatic ether, aliphatic ketone, aliphatic ether, alicyclic or olefin compounds, borate, phosphorate, or a combination of two or more of them.

Examples of aromatic or heteroaromatic-based solvents as described herein include, but not limited to: 1-tetralone, 3-phenoxytoluene, acetophenone, 1-methoxynaphthalene, p-diisopropylbenzene, amylbenzene, tetrahydronaphthalene, cyclohexylbenzene, chloronaphthalene, 1,4-dimethylnaphthalene, 3-isopropylbiphenyl, p-methylcumene, dipentylbenzene, o-diethylbenzene, m-diethylbenzene, p-diethylbenzene, 1,2,3,4-tetramethyl benzene, 1,2,3,5-tetramethyl benzene, 1,2,4,5-tetramethyl benzene, butylbenzene, dodecyl benzene, 1-methylnaphthalene, 1,2,4-trichlorobenzene, 1,3-dipropoxybenzene, 4,4-difluorodiphenylmethane, diphenyl ether, 1,2-dimethoxy-4-(1-propenyl) benzene, diphenylmethane, 2-phenylpyridine, 3-phenylpyridine, 2-phenoxymethyl ether, 2-phenoxytetrahydrofuran, ethyl-2-naphthyl ether, N-methyldiphenylamine, 4-isopropylbiphenyl, a, a-dichlorodiphenylmethane, 4-(3-phenylpropyl) pyridine, benzyl benzoate, 1,1-bis(3,4-dimethylphenyl) ethane, 2-isopropylnaphthalene, dibenzyl ether, etc.

In some embodiments, the suitable and preferred solvents include, but not limited to: aliphatics, alicyclics, aromatics, amines, thiols, amides, nitriles, esters, ethers, polyethers.

The solvent can be a cycloalkane, such as decahydronaphthalene.

In some embodiments, the formulation or another formulation Z2 as described herein comprises at least 50 wt % of an alcoholic solvent; preferably at least 80 wt %; particularly preferably at least 90 wt %.

In some embodiments, the particularly suitable solvent for the present disclosure is a solvent having Hansen solubility parameters in the following ranges:

δ_{d} (dispersion force) is in the range of 17.0 MPa^{1/2} to 23.2 MPa^{1/2}, especially in the range of 18.5 MPa^{1/2} to 21.0 MPa^{1/2}.

δₚ (polarity force) is in the range of 0.2 MPa^{1/2} to 12.5 MPa^{1/2}, especially in the range of 2.0 MPa^{1/2} to 6.0 MPa^{1/2}.

δh (hydrogen bonding force) is in the range of 0.9 MPa^{1/2} to 14.2 MPa^{1/2}, especially in the range of 2.0 MPa^{1/2} to 6.0 MPa^{1/2}.

In the formulation as described herein, the boiling point parameter should be taken into account when selecting the solvents. In the present disclosure, the boiling points of the solvents ≥150° C.; preferably ≥180° C.; more preferably ≥200° C.; further preferably ≥250° C.; and most preferably ≥275° C. or ≥300° C. The boiling points in these ranges are beneficial in terms for preventing nozzle clogging of the inkjet printhead. The solvent can be evaporated from solution system to form a functional film.

In some embodiments, the formulation as described herein, where:
1) the viscosity is in the range of 1 cps to 100 cps at 25° C.; and/or
2) the surface tension is in the range of 19 dyne/cm to 50 dyne/cm at 25° C.

In the formulation as described herein, the surface tension parameter should be taken into account when selecting the resins (prepolymers) or solvents. A suitable surface tension is required for the specific substrates and the specific printing methods. For example, for ink-jet printing, in some embodiments, the surface tension of the resin (prepolymer) or the solvent at 25° C. is in the range of 19 dyne/cm to 50 dyne/cm, further in the range of 22 dyne/cm to 35 dyne/cm, and still further in the range of 25 dyne/cm to 33 dyne/cm.

In some embodiments, the surface tension of the formulation or another formulation Z2 as described herein at 25° C. is in the range of 19 dyne/cm to 50 dyne/cm; more preferably in the range of 22 dyne/cm to 35 dyne/cm; and most preferably in the range of 25 dyne/cm to 33 dyne/cm.

In the formulation or another formulation Z2 as described herein, the viscosity parameters of the ink should be taken into account when selecting the resins (prepolymers) or solvents. The viscosity can be adjusted by different methods, such as by the suitable resin (prepolymer) or solvent and the concentration of functional materials in the ink. In some embodiments, the viscosity of the resin (prepolymer) or solvent is less than 100 cps, further less than 50 cps, and still further from 1.5 cps to 20 cps. The viscosity herein refers to the viscosity during printing at the ambient temperature that is generally at 15° C.-30° C., further 18° C.-28° C., still further 20° C.-25° C., especially 23° C.-25° C. The resulting formulation will be particularly suitable for ink-jet printing.

In some embodiments, the viscosity of the formulation or another formulation Z2 as described herein at 25° C. is in the range of about 1 cps to 100 cps; preferably in the range of 1 cps to 50 cps; and most preferably in the range of 1.5 cps to 20 cps.

The ink obtained from the resin (prepolymer) or the solvent satisfying the above-mentioned boiling point parameter, surface tension parameter and viscosity parameter can form a functional film with uniform thickness and composition property.

In yet another aspect, the present disclosure further provides an organic functional film comprising a formulation as described herein, or formed by using a formulation as described herein.

In yet another aspect, the present disclosure further provides a method for preparing the organic functional film, as shown in the following steps:
1) prepare a formulation as described herein;
2) the formulation is coated on a substrate by printing or coating to form a film, where the method of printing or coating is selected from the group consisting of ink-jet printing, nozzle printing, typographic printing, screen printing, dip coating, spin coating, blade coating, roller printing, torsional roll printing, planographic printing, flexographic printing, rotary printing, spray coating, brush or pad printing, and slit die coating;
3) the obtained film is heated at 50° C. and above, optionally in combination with ultraviolet irradiation, to allow the film to undergo a crosslinking reaction and be cured.

The thickness of the organic functional film is generally 50 nm-100 μm, preferably 100 nm-50 μm, more preferably 300 nm-30 μm, further preferably 300 nm-10 μm, and most preferably 300 nm-4 μm.

In yet another aspect, the present disclosure further provides the applications of the formulation and the organic functional film in optoelectronic devices.

In some embodiments, the optoelectronic device may be selected from a color converter, an organic light-emitting diode (OLED), an organic photovoltaic cell (OPV), an organic light emitting electrochemical cell (OLEEC), an organic light emitting field effect transistor, or an organic laser.

Still further, the present disclosure provides an optoelectronic device comprising a formulation or an organic functional film as described herein.

Preferably, the optoelectronic device is an electroluminescent device, such as a color converter, an organic light-emitting diode (OLED), an organic light emitting electrochemical cell (OLEEC), an organic light emitting field effect transistor, a perovskite light emitting diode (PeLED), and a quantum dot light emitting diode (QD-LED), where one of the functional layers comprises an organic functional film as described herein. The functional layer may be selected from a hole-injection layer, a hole-transport layer, an electron-injection layer, an electron-transport layer, a light-emitting layer, a cathodeic passivation layer (CPL), or an encapsulation layer (TFE).

In some embodiments, the optoelectronic device is an electroluminescent device, comprising two electrodes, the functional layer is located on the same side of the two electrodes.

In some embodiments, the optoelectronic device comprises a light-emitting unit and a color conversion layer (i.e., a functional layer), where the color conversion layer comprises a formulation or an organic functional film as described herein.

In some embodiments, the color conversion layer absorbs less than or equal to 60% of the light from the light-emitting unit, preferably less than or equal to 50%, more preferably less than or equal to 40%, and most preferably less than or equal to 30%. In some embodiments, multi-color light or even white light can be obtained by the color conversion layer. In some embodiments, the color conversion layer absorbs 95% or more of the light from the light-emitting unit, preferably 97% or more, more preferably 99% or more, and most preferably 99.9% or more.

In some embodiments, the light-emitting unit is a solid-state light emitting device. The solid-state light emitting device is preferably selected from a LED, an organic light-emitting diode (OLED), an organic light emitting electrochemical cell (OLEEC), an organic light emitting field effect transistor, a perovskite light emitting diode (PeLED), a quantum dot light emitting diode (QD-LED), or a nanorod LED (see DOI: 10.1038/srep28312).

In some embodiments, the light-emitting unit emits blue light, which is converted into green light or red light by the color conversion layer.

In some embodiments, the light-emitting unit emits green light, which is converted into yellow light or red light by the color conversion layer.

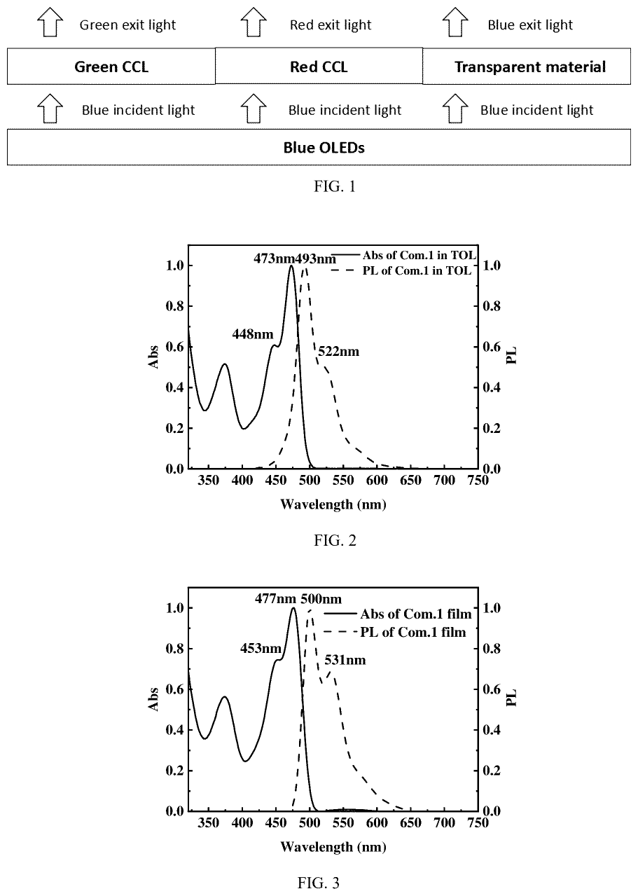

In yet another aspect, the present disclosure further provides a display comprising at least three pixels of red, green and blue. As shown in the FIG. 1, the blue pixel comprises a blue emitting unit, and the pixel of red or green comprises a blue emitting unit and a corresponding red or green color conversion layer.

In some embodiments, the optoelectronic device is an organic light-emitting device comprising a first electrode, an organic light-emitting layer, a second electrode, a color conversion layer, and an encapsulation layer (e.g., an outermost encapsulation layer) in sequence from bottom to top, the second electrode is at least partially transparent, the color conversion layer at least partially absorbs the light emitted by the organic light-emitting layer through the second electrode, where the color conversion layer comprises a formulation as described herein, or formed by using a formulation as described herein. Preferably, the emission spectrum of the organic compound H is on the short wavelength side of the absorption spectrum of the emitter E, and at least partially overlaps with the absorption spectrum of the emitter E. Preferably, the FWHM of the emission spectrum of the emitter E≤55 nm; the light-emitting layer may comprise an organic material, a quantum dot or a perovskite material as a light-emitting material.

The organic light-emitting device may further comprise a substrate, which may be deposited below the first electrode or deposited above the second electrode.

The organic compound H, the emitter E, and the embodiments therefor are as described herein.

In some embodiments, the color conversion layer absorbs less than or equal to 60% of the light emitted by the organic light-emitting layer through the second electrode, preferably less than or equal to 50%, more preferably less than or equal to 40%, and most preferably less than or equal to 30%.

In some embodiments, the color conversion layer absorbs 95% or more of the light emitted by the organic light-emitting layer through the second electrode, preferably 97% or more, more preferably 99% or more, and most preferably 99.9% or more.

In some embodiments, the thickness of the color conversion layer is between 100 nm and 50 μm, preferably between 150 nm and 10 μm, more preferably between 200 nm and 8 μm, further preferably between 200 nm and 6 μm, and most preferably between 200 nm and 4 μm.

In some embodiments, the organic light-emitting device is an OLED. More preferably, the first electrode is an anode, the second electrode is a cathode. Particularly preferably, the organic light-emitting device is a top-emission OLED.

The substrate should be opaque or transparent. A transparent substrate could be used to produce a transparent light-emitting device (for example: Bulovic et al., Nature, 1996, 380, p29, and Gu et al., Appl. Phys. Lett., 1996, 68, p2606). The substrate can be rigid or flexible, e.g. it can be plastic, metal, semiconductor wafer, or glass. Preferably, the substrate has a smooth surface. Particularly ideal are substrates without surface defects. In some embodiments, the substrate is flexible and can be selected from a polymer film or plastic with a glass transition temperature (Tg) >150° C., preferably >200° C., more preferably >250° C., and most preferably >300° C. Examples of the suitable flexible substrates include poly ethylene terephthalate (PET) and polyethylene glycol (2,6-naphthalene) (PEN).

The anode may be a conductive metal, or a metal oxide, or a conductive polymer. The anode should be able to easily inject holes into a hole-injection layer (HIL), a hole-transport layer (HTL), or a light-emitting layer. In some embodiments, the absolute value of the difference between the work function of the anode and the HOMO energy level/valence band energy level of the emitter of the light-emitting layer or the p-type semiconductor materials of the hole-injection layer (HIL)/hole-transport layer (HTL)/electron-blocking layer (EBL)<0.5 eV, preferably <0.3 eV, and most preferably <0.2 eV Examples of anode materials may include, but not limited to: Al, Cu, Au, Ag, Mg, Fe, Co, Ni, Mn, Pd, Pt, ITO, aluminum-doped zinc oxide (AZO), etc. Other suitable anode materials are known and can be readily selected for use by the general technicians in this field. The anode materials can be deposited using any suitable technique, such as a suitable physical vapor deposition method including RF magnetron sputtering, vacuum thermal evaporation, e-beam, etc. In some embodiments, the anode is patterned. Patterned conductive ITO substrates are commercially available and can be used to produce the devices as described herein.

The cathode may be a conductive metal or a metal oxide. The cathode should be able to easily inject electrons into the electron-injection layer (EIL), the electron-transport layer (ETL), or the directly into the light-emitting layer. In some embodiments, the absolute value of the difference between the work function of the cathode and the LUMO energy level/conduction band energy level of the emitter of the light-emitting layer, or the n-type semiconductor materials of the electron-injection layer (EIL)/electron-transport layer (ETL)/hole-blocking layer (HBL)≤0.5 eV, preferably ≤0.3 eV, and most preferably ≤0.2 eV. In principle, all materials those can be used as cathodes for OLEDs may be applied as cathode materials for the devices as described herein. Examples of cathode materials include, but not limited to: Al, Au, Ag, Ca, Ba, Mg, LiF/Al, MgAg alloy, BaF₂/Al, Cu, Fe, Co, Ni, Mn, Pd, Pt, ITO, etc. The cathode materials can be deposited using any suitable technique, such as the suitable physical vapor deposition method including RF magnetron sputtering, vacuum thermal evaporation, e-beam, etc. In some embodiments, the transmittance of the cathode in the range of 400 nm-680 nm ≥40%, preferably ≥45%, more preferably ≥50%, and most preferably ≥60%. Typically, 10 nm-20 nm of Mg:Ag alloys can be used as transparent cathodes, and the ratio of the Mg:Ag can range from 2:8 to 0.5:9.5.

When the organic light-emitting device is an OLED, the light-emitting layer preferably comprises a blue fluorescent host and a blue fluorescent dopant. In some embodiments, the light-emitting layer comprises a blue phosphorescent host and a blue phosphorescent dopant. The OLED may also comprise other functional layers, such as a hole-injection layer (HIL), a hole-transport layer (HTL), an electron-blocking layer (EBL), an electron-injection layer (EIL), an electron-transport layer (ETL), and a hole-blocking layer (HBL). Materials suitable for use in these functional layers are described in details above and in WO2010135519A1, US20090134784A1 and WO2011110277A1. The entire contents of these three documents are hereby incorporated herein for reference.

Further, the electroluminescent device further comprises a cathode capping layer (CPL).

In some embodiments, the CPL is disposed between the second electrode and the color conversion layer.

In some embodiments, the CPL is disposed on the top of the color conversion layer.

The CPL material generally requires a high refractive index (n), such as n ≥1.95@460 nm, preferably ≥1.90@520 nm, more preferably ≥1.85@620 nm. Examples of the CPL materials include:

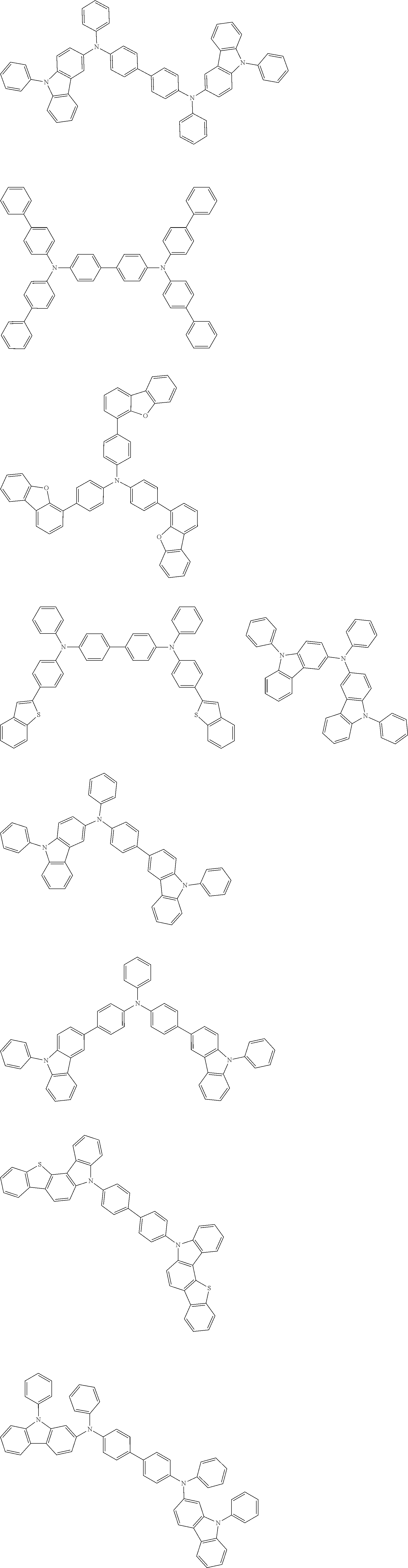

More further examples of the CPL materials can be found in the following patent publications: KR20140128653A, KR20140137231A, KR20140142021A, KR20140142923A, KR20140143618A, KR20140145370A, KR20150004099A, KR20150012835A, U.S. Pat. No. 9,496,520B2, US20150069350A1, CN103828485B, CN104380842B, CN105576143A, TW201506128A, CN103996794A, CN103996795A, CN104744450A, CN104752619A, CN101944570A, US2016308162A1, U.S. Pat. No. 9,095,033B2, US2014034942A1, WO2017014357A1. The above patent documents are incorporated herein by reference in their entirety.

In some embodiments, the color conversion layer comprises a CPL material as described herein. In some embodiments, the color conversion layer is co-evaporated by the above-mentioned CPL material, the organic compound H, and the emitter E. In some embodiments, the mass ratio of the organic compound H is 50%-20%, and the mass ratio of the emitter E is 3%-15%.

Preferably, the encapsulation layer of the organic light-emitting device is thin-film encapsulated (TFE).

In yet another aspect, the present disclosure further provides a display panel, where at least one pixel comprises an organic light-emitting device as described herein.

The organic light-emitting device may be selected from, but not limited to, a color converter, an organic light-emitting diode (OLED), an organic photovoltaic cell (OPV), an organic light emitting electrochemical cell (OLEEC), an organic field effect transistor (OFET), an organic light emitting field effect transistor, an organic laser, an organic spintronic electronic device, an organic sensor, an organic plasmon emitting diode (OPED), etc., particularly preferably an organic electroluminescent device, such as an OLED, an OLEEC, an organic light emitting field effect transistor.

The present disclosure will be described below in conjunction with the preferred embodiments, but the present disclosure is not limited to the following embodiments. It should be understood that the scope of the present disclosure is covered by the scope of the claims of the present disclosure, and those skilled in the art should understand that certain changes may be made to the embodiments of the present disclosure.

### Specific Embodiment

### Example 1: Synthesis Examples of Compounds and Polymers

### 1. Synthesis of Compound 1

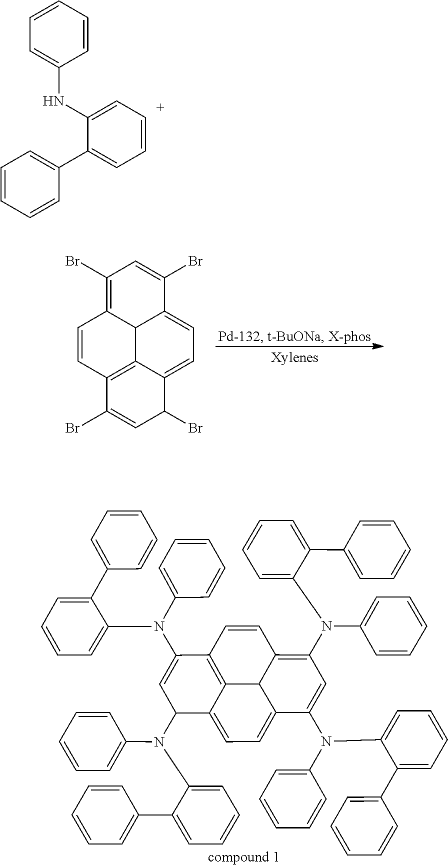 [SMILES: c1ccc(Nc2ccccc2-c2ccccc2)cc1 ; BrC1=CC(Br)=C2C=CC3=C4C(=CC=C1C24)C(Br)C=C3Br ; C1=CC2=C(N(c3ccccc3)c3ccccc3-c3ccccc3)C=C(N(c3ccccc3)c3ccccc3-c3ccccc3)C3=CC=C4C(=C1C(N(c1ccccc1)c1ccccc1-c1ccccc1)=CC4N(c1ccccc1)c1ccccc1-c1ccccc1)C32]

N-phenyl-2-biphenylamine (73.00 g, 297.23 mmol), 1,3,6,8-tetrabromopyrene (34.2 g, 66.05 mmol), Pd-132 (0.94 g, 1.32 mmol), X-Phos (0.94 g), sodium tert-butoxide (25.36 g, 264.19 mmol), and 1.2 L of xylene were added to a 2000 mL dry-clean three-necked flask under N₂ atmosphere. The mixture was heated to 140° C., refluxed for 12 h, then cooled down to room temperature. After the filtration, the filtrate was concentrated, redissolved in the hot toluene, and immediately passed through a thermal insulation silica gel column. After that, the result was concentrated to yield 16.7 g of solid powder, the residue was redissolved in dichloromethane and extracted with saturated brine three times. After the concentration, the organic phases were combined to yield 35 g of crude product, then the obtained crude product was recrystallized with 3 L of xylene to yield 23 g (yield: 29.8%) of compound 1 (solid powder).

### 2. Synthesis of Compound 2

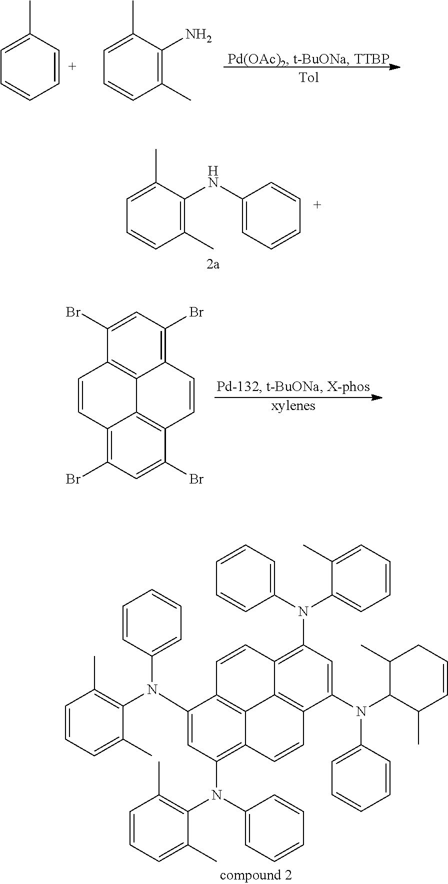

Iodobenzene (50.00 g, 245.1 mmol), 2,6-dimethylaniline (29.68 g, 245.1 mmol), palladium acetate (0.56 g, 2.45 mmol), tri-tert-butylphosphine (1 mL), sodium tert-butoxide (47.06 g, 490.2 mmol), and toluene (500 mL) were added to a 1000 mL dry-clean three-necked flask under N₂ atmosphere. The mixture was heated to 100° C., refluxed for 5 h, then cooled down to room temperature. After extracting with ethyl acetate and saturated brine three times, the concentrated organic phase was passed through a short silica gel column (eluent: dichloromethane:n-hexane=1:10), then the result was concentrated to yield 45 g (yield: 93.7%) of intermediate 2a (black-brown solid).

Intermediate 2a (39.00 g, 197.3 mmol), 1,3,6,8-tetrabromopyrene (22.7 g, 48.3 mmol), Pd-132 (0.62 g, 0.97 mmol), X-Phos (0.62 g), sodium tert-butoxide (16.8 g, 175.3 mmol), and 1 L of xylene were added to a 2000 mL dry-clean three-necked flask under N₂ atmosphere. The mixture was heated to 140° C., refluxed for 12 h, then cooled down to room temperature. After extracting with ethyl acetate and saturated brine three times, the organic phase was concentrated, then the result was redissolved in the hot toluene, and immediately passed through a thermal insulation silica gel column. After the collection, the filtrate was concentrated, and washed with n-hexane. The sample was then filtrated and the residue was further washed with n-hexane to yield 5.1 g of crude product. After that, the obtained crude product was washed by heated tetrahydrofuran, then the result was filtrated while hot to yield 1 g (yield: 2.1%) of compound 2 (solid powder).

### 3. Synthesis of Compound 3

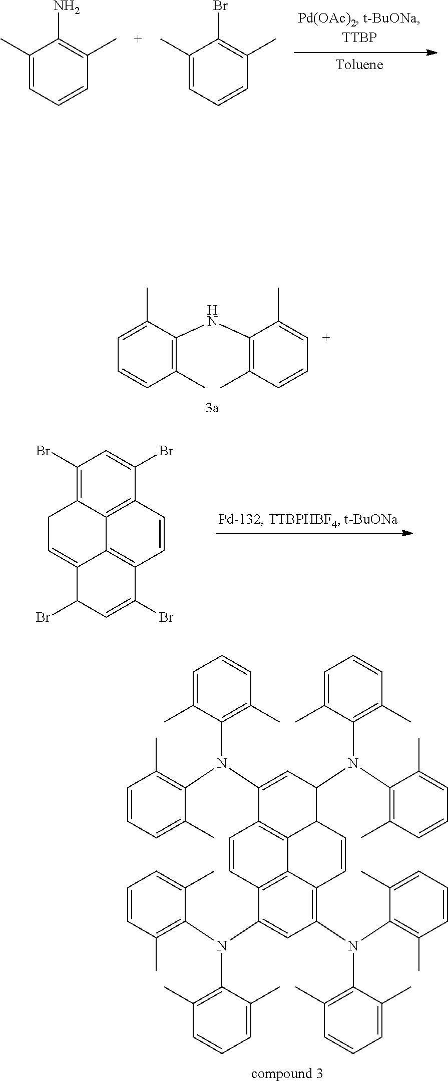

2,6-Dimethylaniline (50.00 g, 413.2 mmol), 2-bromo-m-xylene (76 g, 413.2 mmol), palladium acetate (0.46 g, 2.05 mmol), tri-tert-butylphosphine (1 mL), sodium tert-butoxide (79.3 g, 826.04 mmol), and toluene (500 mL) were added to a 1000 mL dry-clean three-necked flask under N₂ atmosphere. The mixture was heated to 100° C., refluxed for 2.5 h, then cooled down to room temperature. After extracting with ethyl acetate and saturated brine three times, the concentrated organic phase was passed through a short silica gel column (eluent: dichloromethane:n-hexane=1:10), then the result was concentrated to yield 88 g (yield: 94.6%) of intermediate 3a (solid).

Intermediate 3a (48.9 g, 217.33 mmol), 1,3,6,8-tetrabromopyrene (25 g, 48.3 mmol), Pd-132 (1.67 g, 2.35 mmol), tri-tert-butylphosphine tetrafluoroborate (1.67 g), sodium tert-butoxide (18.54 g, 193.13 mmol), and 1 L of xylene were added to a 2000 mL dry-clean three-necked flask under N₂ atmosphere. The mixture was heated to 140° C., refluxed for 12 h, then cooled down to room temperature. After extracting with dichloromethane and saturated brine three times, the organic phase was concentrated, then the result was redissolved in the hot toluene, and immediately passed through a thermal insulation silica gel column. After the collection, the filtrate was concentrated, and washed with n-hexane. Then the sample was filtrated and the residue was further washed with n-hexane to yield 5 g (yield: 9.5%) of compound 3 (solid powder).

### 4. Synthesis of Compound 4

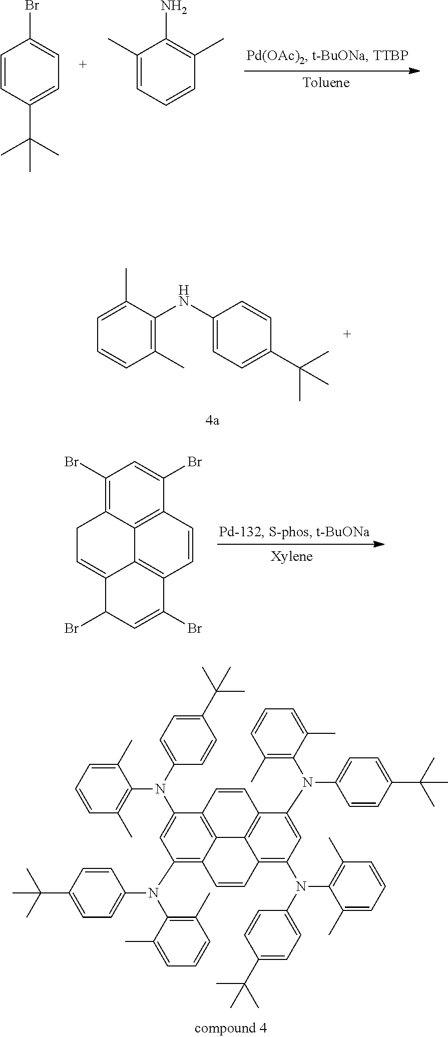

2,6-Dimethylaniline (50.00 g, 413.2 mmol), 1-bromo-4-tert-butylbenzene (88.00 g, 413.2 mmol), palladium acetate (0.46 g, 2.05 mmol), tri-tert-butylphosphine (1 mL), sodium tert-butoxide (59.5 g, 619.83 mmol), and toluene (500 mL) were added to a 1000 mL dry-clean three-necked flask under N₂ atmosphere. The mixture was heated to 100° C., refluxed for 2.5 h, then cooled down to room temperature. After extracting with ethyl acetate and saturated brine three times, the concentrated organic phase was passed through a short silica gel column (eluent: dichloromethane:n-hexane=1:10), then the result was concentrated to yield 93 g (yield: 89.4%) of intermediate 4a (solid).

Intermediate 4a (43.97 g, 173.79 mmol), 1,3,6,8-tetrabromopyrene (20 g, 38.62 mmol), Pd-132 (0.82 g, 1.16 mmol), S-Phos (0.82 g), sodium tert-butoxide (14.83 g, 154.48 mmol), and 1 L of xylene were added to a 2000 mL dry-clean three-necked flask under N₂ atmosphere. The mixture was heated to 140° C., refluxed for 12 h, then cooled down to room temperature. After extracting with dichloromethane and saturated brine three times, the organic phase was concentrated, then the result was redissolved in the hot toluene, and immediately passed through a thermal insulation silica gel column. After the collection, the filtrate was concentrated to yield 25 g of crude product, then the obtained crude product was recrystallized with xylene to yield 20 g (yield: 42.8%) of compound 4 (solid powder).

### 5. Synthesis of Compound 5

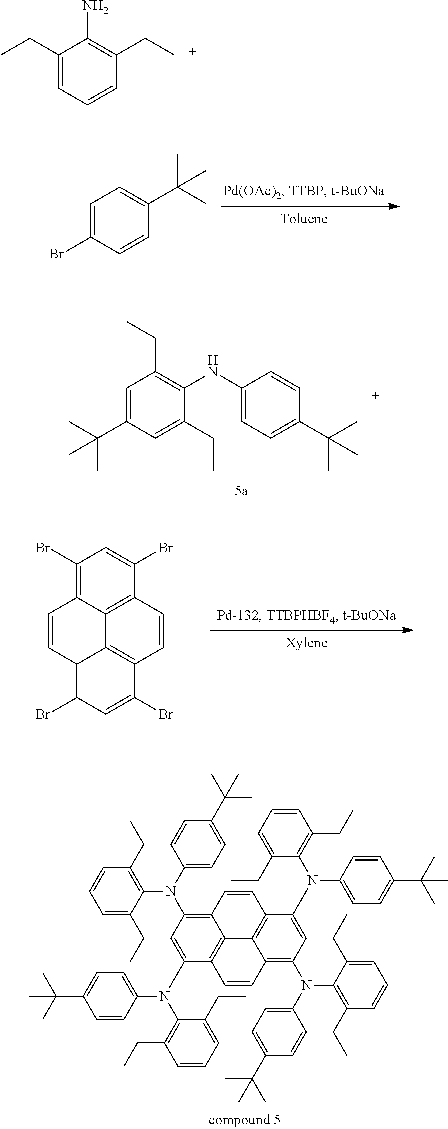

4-Tert-butylaniline (23.94 g, 159.6 mmol), 1-bromo-4-tert-butylbenzene (34.00 g, 159.6 mmol), palladium acetate (0.18 g, 0.8 mmol), tri-tert-butylphosphine (0.5 mL), sodium tert-butoxide (22.98 g, 239.4 mmol), and toluene (500 mL) were added to a 1000 mL dry-clean three-necked flask under N₂ atmosphere. The mixture was heated to 100° C., refluxed for 2.5 h, then cooled down to room temperature. After extracting with ethyl acetate and saturated brine three times, the concentrated organic phase was passed through a short silica gel column (eluent: dichloromethane: n-hexane=1:10), then the result was concentrated to yield 12 g (yield: 27.1%) of intermediate 5a (solid).

Intermediate 5a (10 g, 35.59 mmol), 1,3,6,8-tetrabromopyrene (4 g, 7.72 mmol), Pd-132 (0.27 g, 0.38 mmol), S-Phos (0.27 g, equivalent to the catalyst), sodium tert-butoxide (3 g, 31.25 mmol), and 100 mL of xylene were added to a 250 mL dry-clean three-necked flask under N₂ atmosphere. The mixture was heated to 140° C., refluxed for 12 h, then cooled down to room temperature. After extracting with dichloromethane and saturated brine three times, the organic phase was concentrated, then the result was redissolved in the hot xylene, and immediately passed through a thermal insulation silica gel column. After that, the filtrate was concentrated to yield 4 g (yield: 39.2%) of compound 5 (solid powder).

### 6. Synthesis of Compound 6

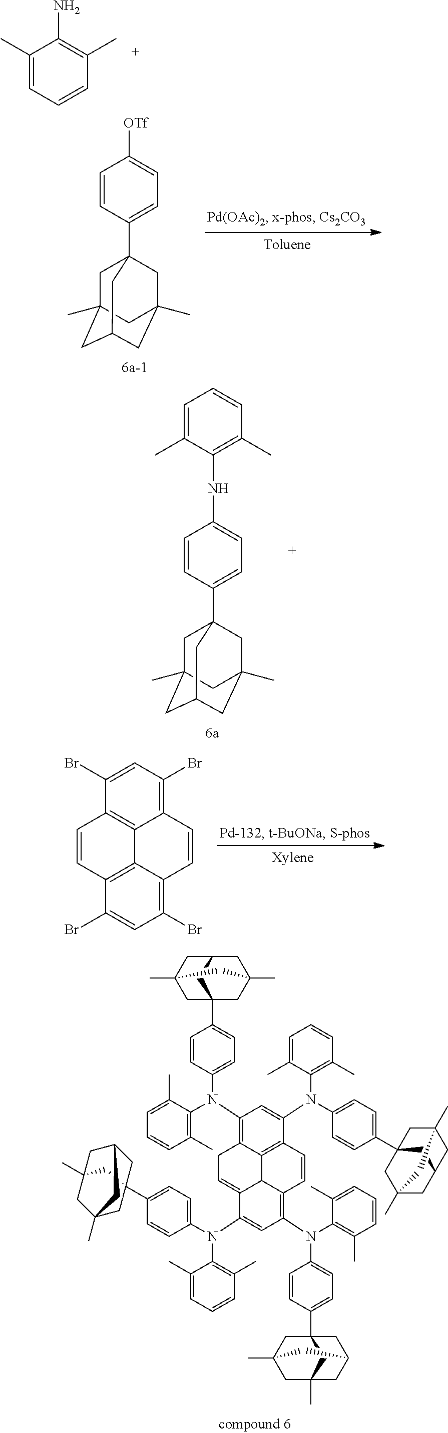

2,6-Dimethylaniline (14.85 g, 122.73 mmol), 6a-1 (50.00 g, 128.87 mmol), palladium acetate (0.28 g, 1.25 mmol), X-Phos (0.28 g), cesium carbonate (59.98 g, 184.09 mmol), and toluene (500 mL) were added to a 1000 mL dry-clean three-necked flask under N₂ atmosphere. The mixture was heated to 100° C., refluxed for 3.5 h, then cooled down to room temperature. After extracting with ethyl acetate and saturated brine three times, the concentrated organic phase was passed through a short silica gel column (eluent: dichloromethane:n-hexane=1:10), then the result was concentrated to yield 18 g (yield: 40.9%) of intermediate 6a (solid).

Intermediate 6a (18 g, 50.14 mmol), 1,3,6,8-tetrabromopyrene (5.77 g, 11.14 mmol), Pd-132 (0.16 g, 0.23 mmol), S-Phos (0.16 g), sodium tert-butoxide (4.28 g, 44.58 mmol), and 250 mL of xylene were added to a 500 mL dry-clean three-necked flask under N₂ atmosphere. The mixture was heated to 140° C., refluxed for 14 h, then cooled down to room temperature. After extracting with dichloromethane and saturated brine three times, the combined organic phase was concentrated to yield crude product, then the obtained crude product was separated and purified by silica gel column chromatography (eluent: dichloromethane:petroleum ether=1:20) to yield 1.8 g (yield: 9.9%) of compound 6 (solid powder).

### 7. Synthesis of Compound 7

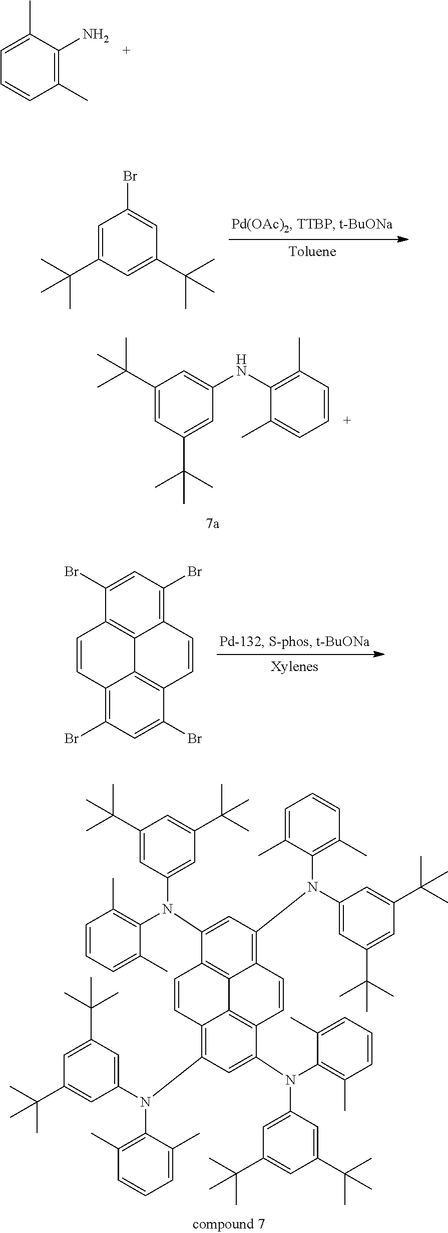

2,6-Dimethylaniline (21 g, 173.55 mmol), 3,5-di-tert-butylbromobenzene (47.00 g, 174.72 mmol), palladium acetate (0.19 g, 0.85 mmol), tri-tert-butylphosphine (1 mL), sodium tert-butoxide (24.09 g, 250.09 mmol), and toluene (500 mL) were added to a 1000 mL dry-clean three-necked flask under N₂ atmosphere. The mixture was heated to 100° C., refluxed for 12 h, then cooled down to room temperature. After extracting with ethyl acetate and saturated brine three times, the concentrated organic phase was passed through a short silica gel column (eluent: dichloromethane:n-hexane=1:10), then the result was concentrated to yield 48 g (yield: 89.72%) of intermediate 7a (oily substance).

Intermediate 7a (53 g, 171.52 mmol), 1,3,6,8-tetrabromopyrene (19.74 g, 38.12 mmol), Pd-132 (0.54 g, 0.76 mmol), S-Phos (0.54 g), sodium tert-butoxide (14.64 g, 152.49 mmol), and 1 L of xylene were added to a 2000 mL dry-clean three-necked flask under N₂ atmosphere. The mixture was heated to 140° C., refluxed for 14 h, then cooled down to room temperature. After extracting with dichloromethane and saturated brine three times, the organic phase was concentrated, then the result was redissolved in the hot toluene, and immediately passed through a thermal insulation silica gel column. After the collection, the filtrate was concentrated to yield 30 g of crude product, then the obtained crude product was recrystallized with tetrahydrofuran to yield 22.5 g (yield: 41.3%) of compound 7 (solid powder).

### 8. Synthesis of Compound 8

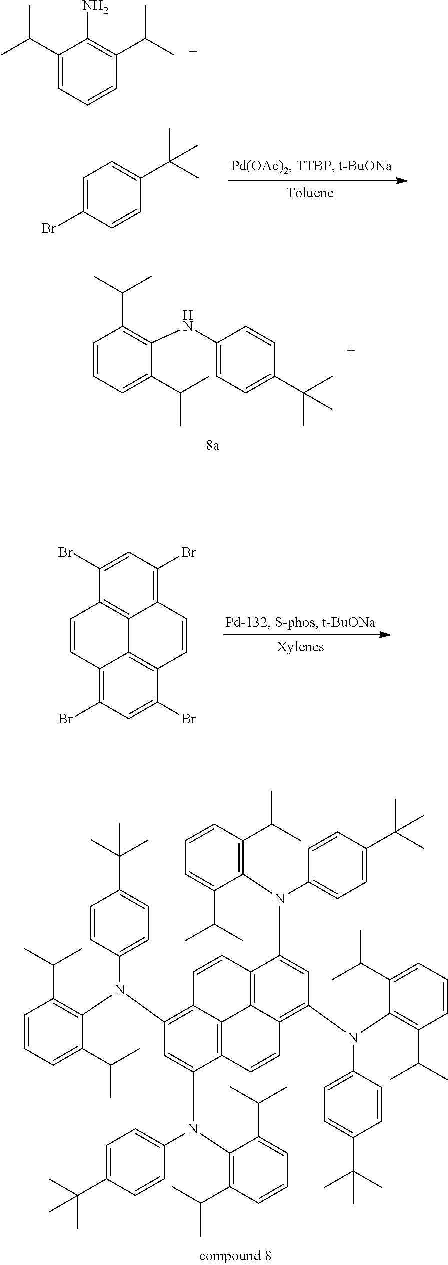 [SMILES: CC(C)c1cccc(C(C)C)c1N ; CC(C)(C)c1ccc(Br)cc1 ; CC(C)c1cccc(C(C)C)c1Nc1ccc(C(C)(C)C)cc1 ; Brc1cc(Br)c2ccc3c(Br)cc(Br)c4ccc1c2c43 ; CC(C)c1cccc(C(C)C)c1N(c1ccc(C(C)(C)C)cc1)c1cc(N(c2ccc(C(C)(C)C)cc2)c2c(C(C)C)cccc2C(C)C)c2ccc3c(N(c4ccc(C(C)(C)C)cc4)c4c(C(C)C)cccc4C(C)C)cc(N(c4ccc(C(C)(C)C)cc4)c4c(C(C)C)cccc4C(C)C)c4ccc1c2c43]

2,6-Diisopropylaniline (24.9 g, 140.68 mmol), 1-bromo-4-tert-butylbenzene (30.00 g, 140.85 mmol), palladium acetate (0.16 g, 0.71 mmol), tri-tert-butylphosphine (1 mL), sodium tert-butoxide (20.3 g, 211.46 mmol), and toluene (500 mL) were added to a 1000 mL dry-clean three-necked flask under N₂ atmosphere. The mixture was heated to 100° C., refluxed for 12 h, then cooled down to room temperature. After extracting with ethyl acetate and saturated brine three times, the concentrated organic phase was passed through a short silica gel column (eluent: dichloromethane:n-hexane=1:10), then the result was concentrated to yield 38 g (yield: 86.9%) of intermediate 8a (oily substance).

Intermediate 8a (38 g, 122.98 mmol), 1,3,6,8-tetrabromopyrene (14.15 g, 27.33 mmol), Pd-132 (0.39 g, 0.55 mmol), S-Phos (0.39 g), sodium tert-butoxide (10.49 g, 109.27 mmol), and 1 L of xylene were added to 2000 mL dry-clean three-necked flask under N₂ atmosphere. The mixture was heated to 140° C., refluxed for 14 h, then cooled down to room temperature. After extracting with dichloromethane and saturated brine three times, the combined organic phase was concentrated, then the resulting sample was separated and purified by silica gel column chromatography (eluent: dichloromethane:petroleum ether=1:20) to yield 3.9 g (yield: 10%) of compound 8 (solid powder).

### 9. Synthesis of Compound 9

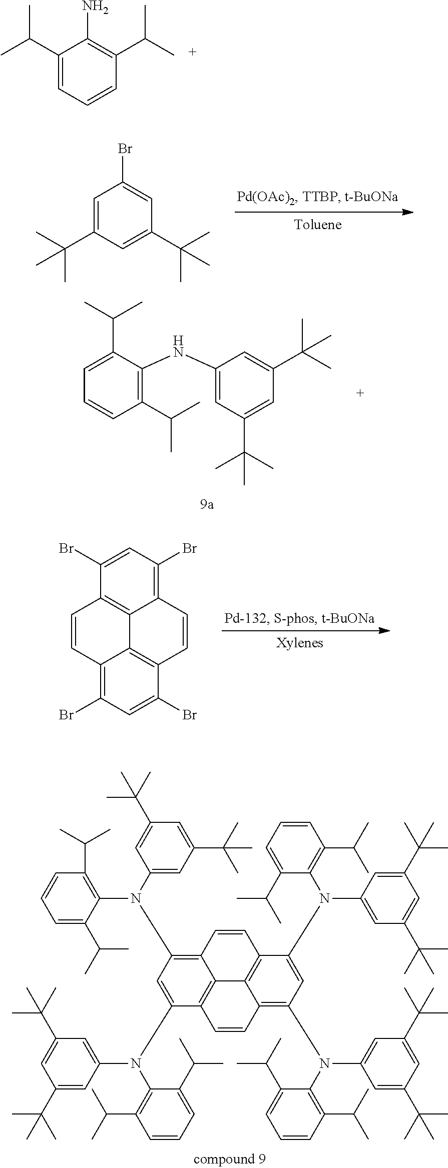

2,6-Diisopropylaniline (40 g, 225.99 mmol), 3,5-di-tert-butylbromobenzene (60.79 g, 226.82 mmol), palladium acetate (0.25 g, 1.11 mmol), tri-tert-butylphosphine (1.5 mL), sodium tert-butoxide (32.5 g, 338.54 mmol), and toluene (500 mL) were added to a 1000 mL dry-clean three-necked flask under N₂ atmosphere. The mixture was heated to 100° C., refluxed for 12 h, then cooled down to room temperature. After extracting with ethyl acetate and saturated brine three times, the concentrated organic phase was passed through a short silica gel column (eluent: dichloromethane:n-hexane=1:10), then the result was concentrated to yield 76 g (yield: 92.1%) of intermediate 9a (solid).

Intermediate 9a (76 g, 208.22 mmol), 1,3,6,8-tetrabromopyrene (23.96 g, 46.27 mmol), Pd-132 (0.66 g, 0.93 mmol), S-Phos (0.66 g), sodium tert-butoxide (17.77 g, 185.10 mmol.), and 1 L of xylene were added to a 2000 mL dry-clean three-necked flask under N₂ atmosphere. The mixture was heated to 140° C., refluxed for 14 h, then cooled down to room temperature. After extracting with dichloromethane and saturated brine three times, the combined organic phase was concentrated, then the resulting sample was separated and purified by silica gel column chromatography (eluent: dichloromethane:petroleum ether=1:20) to yield 7 g (yield: 9.1%) of compound 9 (solid powder).

### 10. Synthesis of Compound 10

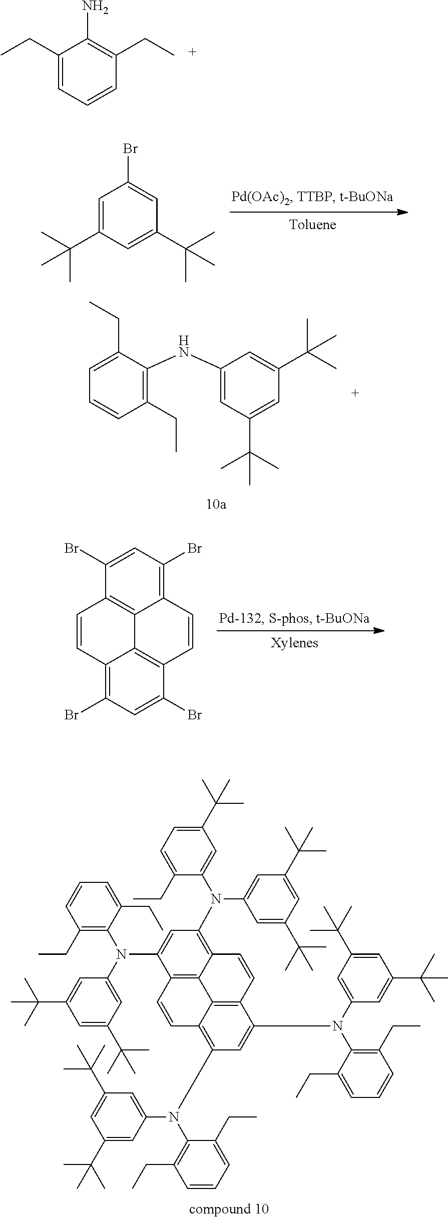

2,6-Diethylaniline (24.9 g, 167.11 mmol), 3,5-di-tert-butylbromobenzene (43 g, 160.45 mmol), palladium acetate (0.19 g, 0.85 mmol), tri-tert-butylphosphine (1 mL), sodium tert-butoxide (24 g, 250.0 mmol), and toluene (500 mL) were added to a 1000 mL dry-clean three-necked flask under N₂ atmosphere. The mixture was heated to 100° C., refluxed for 12 h, then cooled down to room temperature. After extracting with ethyl acetate and saturated brine three times, the combined organic phase was passed through a short silica gel column (eluent: dichloromethane:n-hexane=1:10), then the result was concentrated to yield 52 g (yield: 96.3%) of intermediate 10a (oily substance).

Intermediate 10a (52 g, 154.30 mmol), 1,3,6,8-tetrabromopyrene (17.76 g, 34.30 mmol), Pd-132 (0.49 g, 0.69 mmol), S-Phos (0.49 g), sodium tert-butoxide (13.17 g, 137.19 mmol), and 1 L of xylene were added to a 2000 mL dry-clean three-necked flask under N₂ atmosphere. The mixture was heated to 140° C., refluxed for 14 h, then cooled down to room temperature. After extracting with dichloromethane and saturated brine three times, the combined organic phase was concentrated, then the result was redissolved in the hot toluene, and immediately passed through a thermal insulation silica gel column. After the collection, the filtrate was concentrated to yield 15 g of crude product, then the obtained crude product was recrystallized with tetrahydrofuran to yield 9.6 g (yield: 18.5%) of compound 10 (solid powder).

### 11. Synthesis of Compound 11

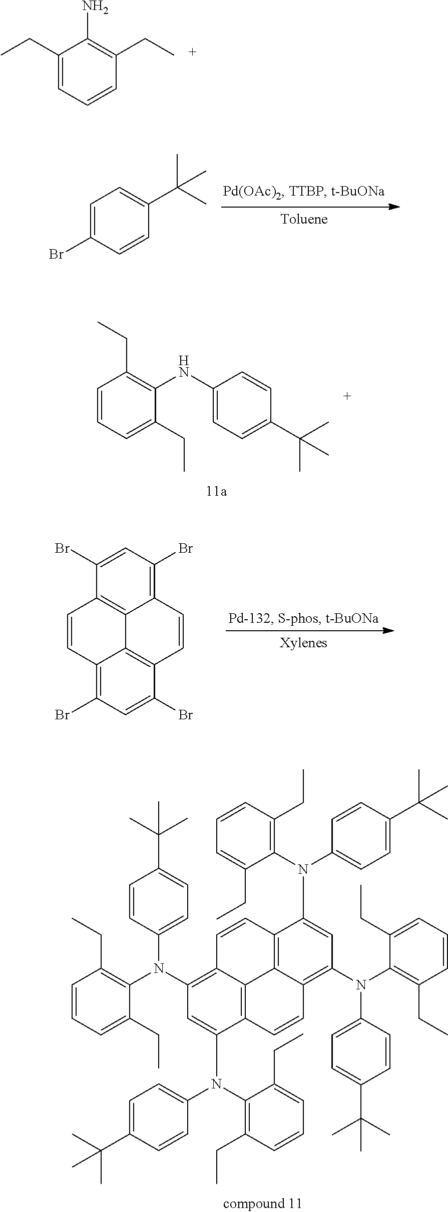 [SMILES: CCc1cccc(CC)c1N ; CC(C)(C)c1ccc(Br)cc1 ; CCc1cccc(CC)c1Nc1ccc(C(C)(C)C)cc1 ; Brc1cc(Br)c2ccc3c(Br)cc(Br)c4ccc1c2c43 ; CCc1cccc(CC)c1N(c1ccc(C(C)(C)C)cc1)c1cc(N(c2ccc(C(C)(C)C)cc2)c2c(CC)cccc2CC)c2ccc3c(N(c4ccc(C(C)(C)C)cc4)c4c(CC)cccc4CC)cc(N(c4ccc(C(C)(C)C)cc4)c4c(CC)cccc4CC)c4ccc1c2c43]

2,6-Diethylaniline (34.98 g, 234.74 mmol), 1-bromo-4-tert-butylbenzene (50 g, 234.74 mmol), palladium acetate (0.26 g, 1.16 mmol), tri-tert-butylphosphine (1.5 mL), sodium tert-butoxide (33.8 g, 352.08 mmol), and toluene (500 mL) were added to a 1000 mL dry-clean three-necked flask under N₂ atmosphere. The mixture was heated to 100° C., refluxed for 12 h, then cooled down to room temperature. After extracting with ethyl acetate and saturated brine three times, the combined organic phase was passed through a short silica gel column (eluent: dichloromethane:n-hexane=1:10), then the solvent was removed to yield 60 g (yield: 90.9%) of intermediate 11a (oily substance).

Intermediate 11a (70 g, 249.11 mmol), 1,3,6,8-tetrabromopyrene (30 g, 57.92 mmol), Pd-132 (0.81 g, 1.14 mmol), S-Phos (0.81 g), sodium tert-butoxide (21.86 g, 227.71 mmol), and 1 L of xylene were added to a2000 mL dry-clean three-necked flask under N₂ atmosphere. The mixture was heated to 140° C., refluxed for 14 h, then cooled down to room temperature. After extracting with dichloromethane and saturated brine three times, the result was redissolved in the hot toluene, and immediately passed through a thermal insulation silica gel column. After the collection, the filtrate was concentrated to yield 10 g of crude product, then the obtained crude product was recrystallized with tetrahydrofuran to yield 4 g (yield: 5.2%) of compound 11 (solid powder).

### Other Materials:

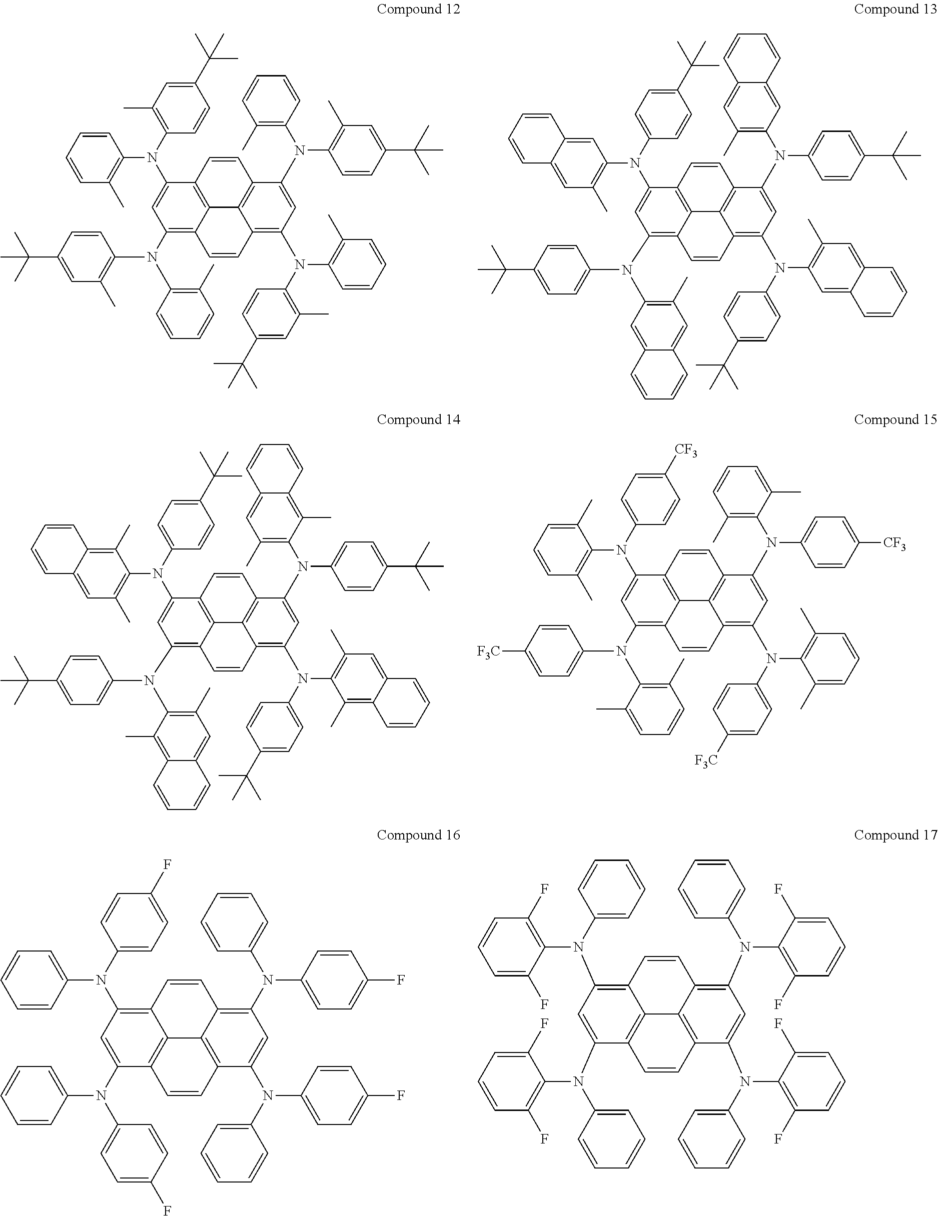

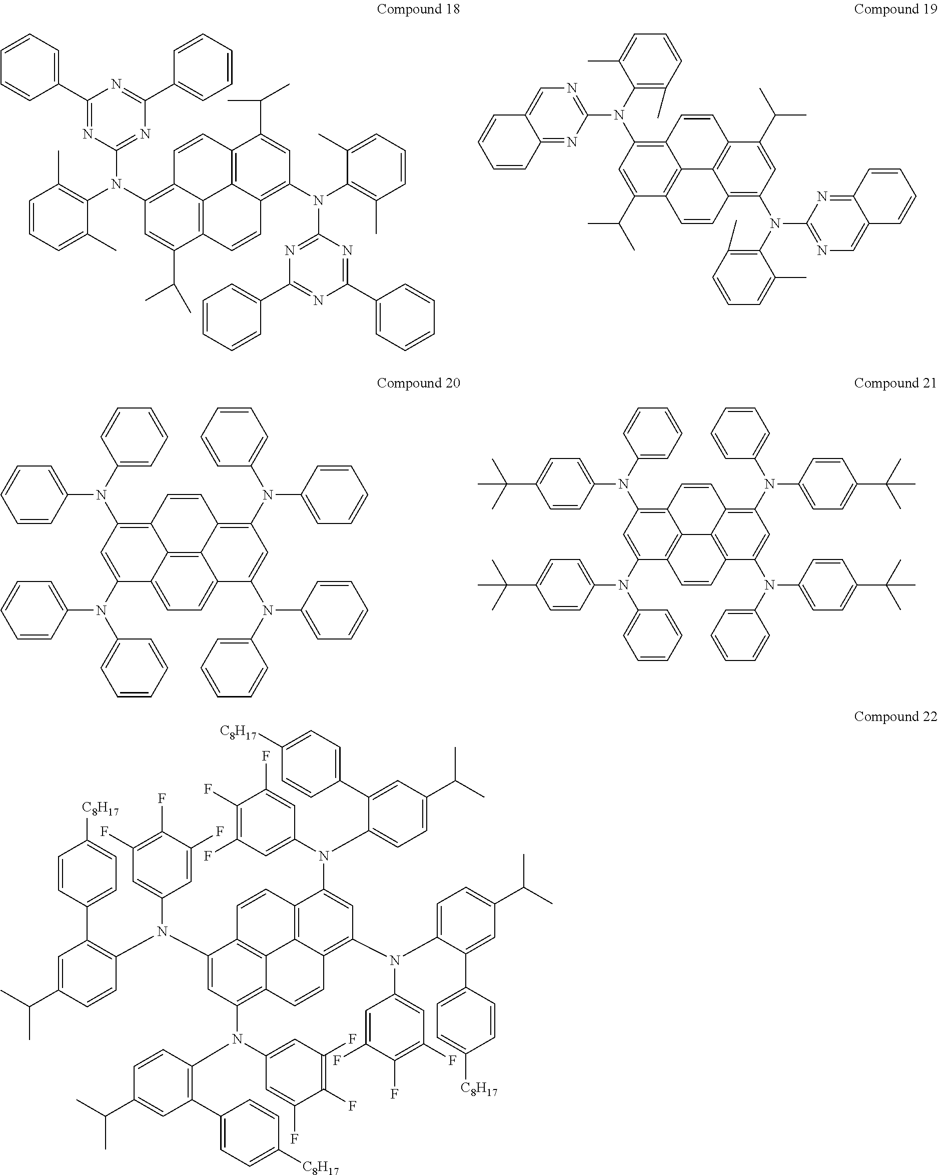

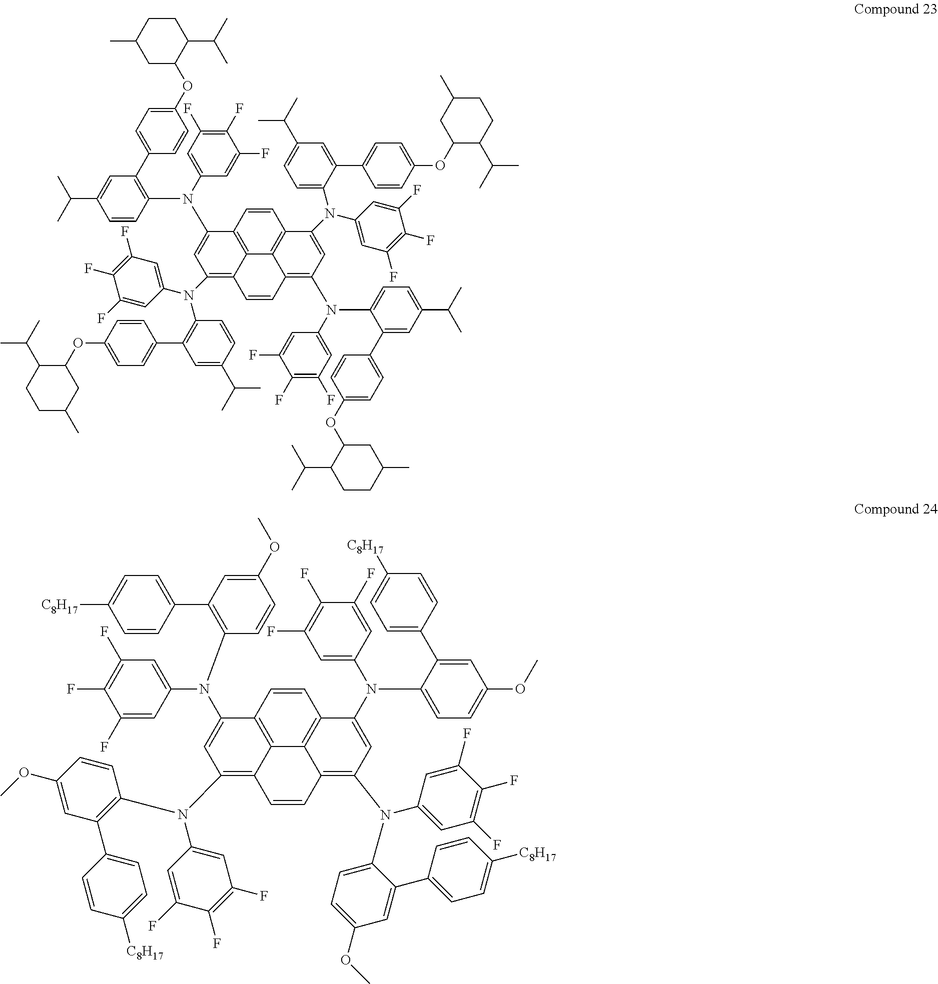

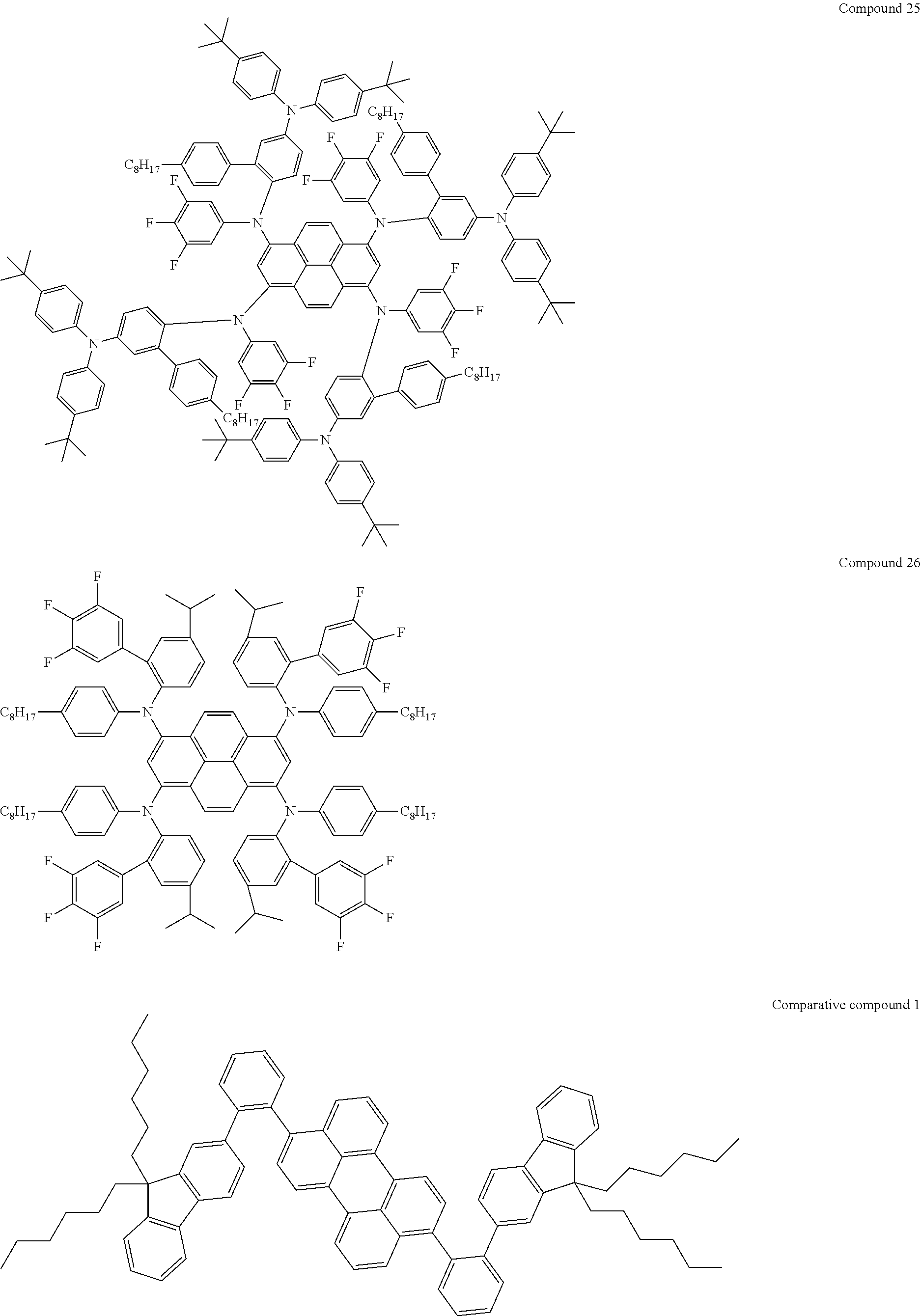

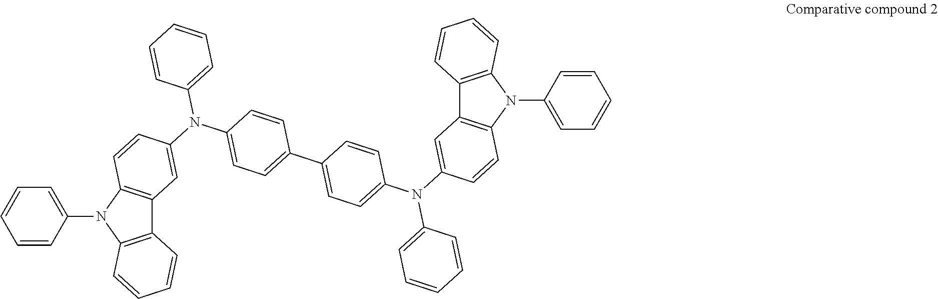 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

The synthesis of compound 12-compound 26 is similar to that ofcompound 1-compound 11. The comparative compound 1 was synthesized according to patent cooperation treaty (International Application Publication No. WO2022213993A1), and the comparative compound 2 was synthesized according to US20150069350A1.

| Mate- | | Raw Materials | Raw Materials | Raw Materials |
|---|---|---|---|---|
| rials | Target Structures | 1 | 2 | 3 |
| Com- pound 12 | 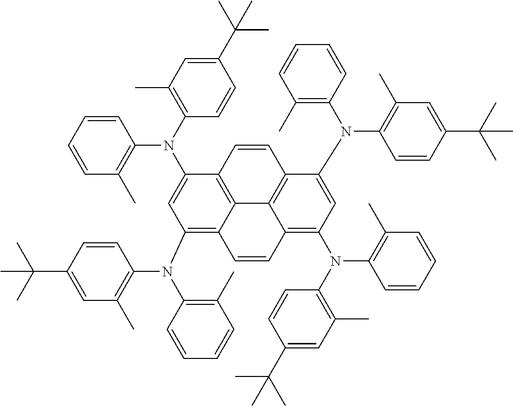 [SMILES: Cc1ccccc1N(c1ccc(C(C)(C)C)cc1C)c1cc(N(c2ccccc2C)c2ccc(C(C)(C)C)cc2C)c2ccc3c(N(c4ccccc4C)c4ccc(C(C)(C)C)cc4C)cc(N(c4ccccc4C)c4ccc(C(C)(C)C)cc4C)c4ccc1c2c43] | 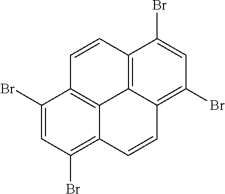 [SMILES: Brc1cc(Br)c2ccc3c(Br)cc(Br)c4ccc1c2c43] | 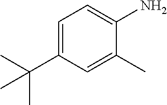 | 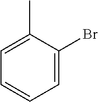 |
| Com- pound 13 | 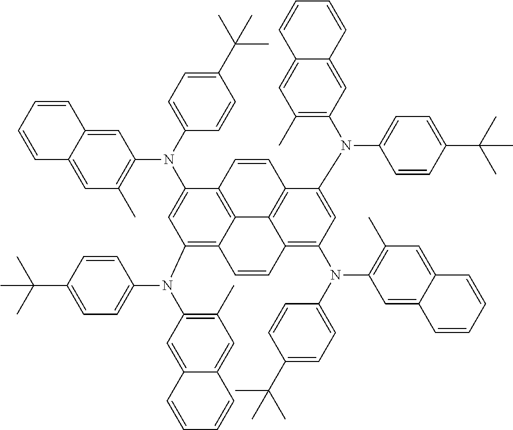 [SMILES: Cc1cc2ccccc2cc1N(c1ccc(C(C)(C)C)cc1)c1cc(N(c2ccc(C(C)(C)C)cc2)c2cc3ccccc3cc2C)c2ccc3c(N(c4ccc(C(C)(C)C)cc4)c4cc5ccccc5cc4C)cc(N(c4ccc(C(C)(C)C)cc4)c4cc5ccccc5cc4C)c4ccc1c2c43] | 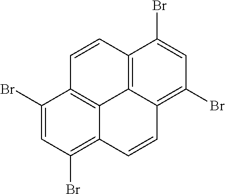 [SMILES: Brc1cc(Br)c2ccc3c(Br)cc(Br)c4ccc1c2c43] | 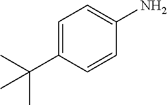 | 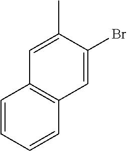 |
| Com- pound 14 | 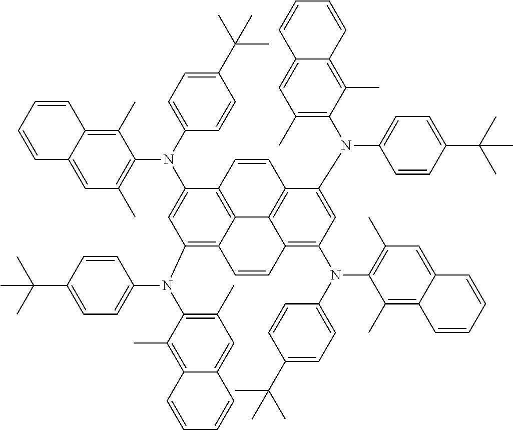 [SMILES: Cc1cc2ccccc2c(C)c1N(c1ccc(C(C)(C)C)cc1)c1cc(N(c2ccc(C(C)(C)C)cc2)c2c(C)cc3ccccc3c2C)c2ccc3c(N(c4ccc(C(C)(C)C)cc4)c4c(C)cc5ccccc5c4C)cc(N(c4ccc(C(C)(C)C)cc4)c4c(C)cc5ccccc5c4C)c4ccc1c2c43] | 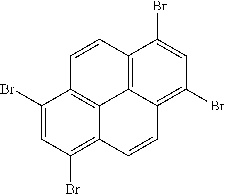 [SMILES: Brc1cc(Br)c2ccc3c(Br)cc(Br)c4ccc1c2c43] | 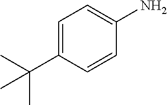 | 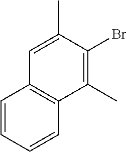 |
| Com- pound 15 | 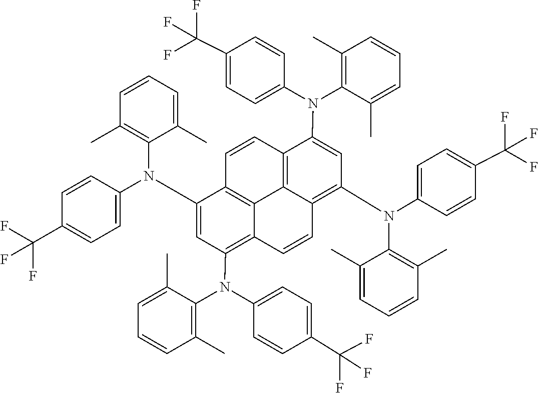 [SMILES: Cc1cccc(C)c1N(c1ccc(C(F)(F)F)cc1)c1cc(N(c2ccc(C(F)(F)F)cc2)c2c(C)cccc2C)c2ccc3c(N(c4ccc(C(F)(F)F)cc4)c4c(C)cccc4C)cc(N(c4ccc(C(F)(F)F)cc4)c4c(C)cccc4C)c4ccc1c2c43] | 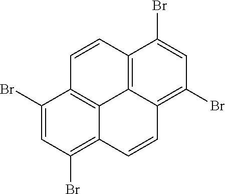 [SMILES: Brc1cc(Br)c2ccc3c(Br)cc(Br)c4ccc1c2c43] | 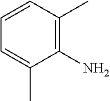 | 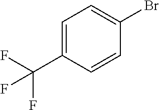 [SMILES: FC(F)(F)c1ccc(Br)cc1] |
| Com- pound 16 | 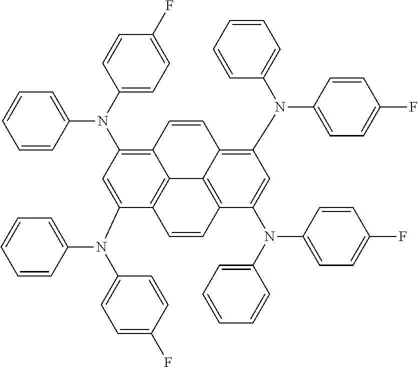 [SMILES: Fc1ccc(N(c2ccccc2)c2cc(N(c3ccccc3)c3ccc(F)cc3)c3ccc4c(N(c5ccccc5)c5ccc(F)cc5)cc(N(c5ccccc5)c5ccc(F)cc5)c5ccc2c3c54)cc1] | 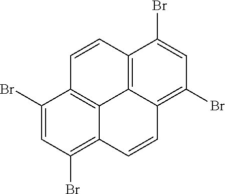 [SMILES: Brc1cc(Br)c2ccc3c(Br)cc(Br)c4ccc1c2c43] | 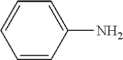 | 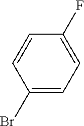 |
| Com- pound 17 | 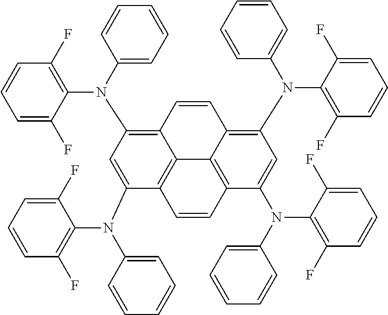 [SMILES: Fc1cccc(F)c1N(c1ccccc1)c1cc(N(c2ccccc2)c2c(F)cccc2F)c2ccc3c(N(c4ccccc4)c4c(F)cccc4F)cc(N(c4ccccc4)c4c(F)cccc4F)c4ccc1c2c43] | 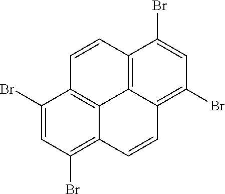 [SMILES: Brc1cc(Br)c2ccc3c(Br)cc(Br)c4ccc1c2c43] | 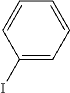 | 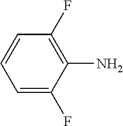 |
| Com- pound 18 | 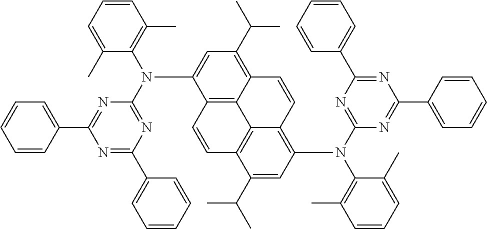 [SMILES: Cc1cccc(C)c1N(c1nc(-c2ccccc2)nc(-c2ccccc2)n1)c1cc(C(C)C)c2ccc3c(N(c4nc(-c5ccccc5)nc(-c5ccccc5)n4)c4c(C)cccc4C)cc(C(C)C)c4ccc1c2c43] | 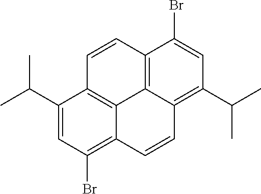 [SMILES: CC(C)c1cc(Br)c2ccc3c(C(C)C)cc(Br)c4ccc1c2c43] | 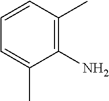 | 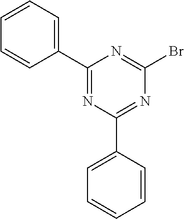 [SMILES: Brc1nc(-c2ccccc2)nc(-c2ccccc2)n1] |
| Com- pound 19 | 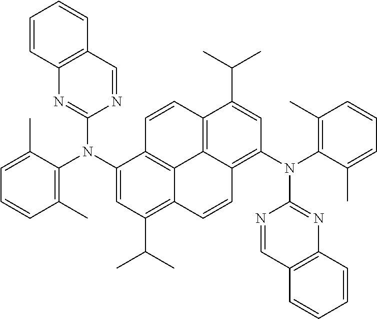 [SMILES: Cc1cccc(C)c1N(c1ncc2ccccc2n1)c1cc(C(C)C)c2ccc3c(N(c4ncc5ccccc5n4)c4c(C)cccc4C)cc(C(C)C)c4ccc1c2c43] | 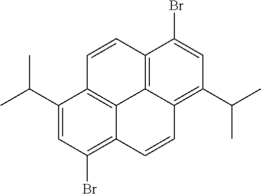 [SMILES: CC(C)c1cc(Br)c2ccc3c(C(C)C)cc(Br)c4ccc1c2c43] | 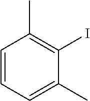 | 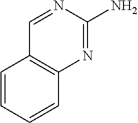 [SMILES: Nc1ncc2ccccc2n1] |
| Com- pound 20 | 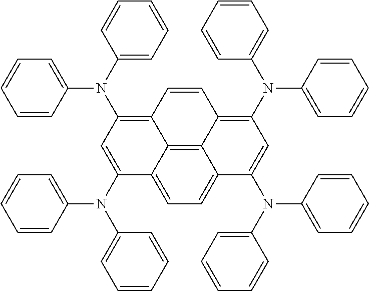 [SMILES: c1ccc(N(c2ccccc2)c2cc(N(c3ccccc3)c3ccccc3)c3ccc4c(N(c5ccccc5)c5ccccc5)cc(N(c5ccccc5)c5ccccc5)c5ccc2c3c54)cc1] | 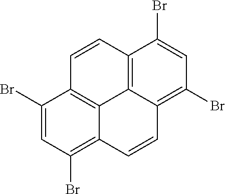 [SMILES: Brc1cc(Br)c2ccc3c(Br)cc(Br)c4ccc1c2c43] | 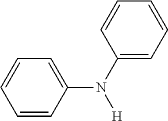 [SMILES: c1ccc(Nc2ccccc2)cc1] | |
| Com- pound 21 | 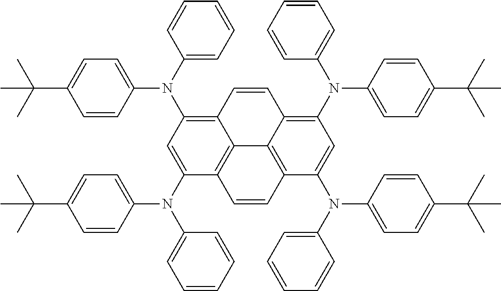 [SMILES: CC(C)(C)c1ccc(N(c2ccccc2)c2cc(N(c3ccccc3)c3ccc(C(C)(C)C)cc3)c3ccc4c(N(c5ccccc5)c5ccc(C(C)(C)C)cc5)cc(N(c5ccccc5)c5ccc(C(C)(C)C)cc5)c5ccc2c3c54)cc1] | 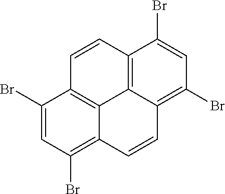 [SMILES: Brc1cc(Br)c2ccc3c(Br)cc(Br)c4ccc1c2c43] | 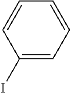 | 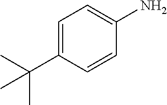 |
| Com- pound 22 | 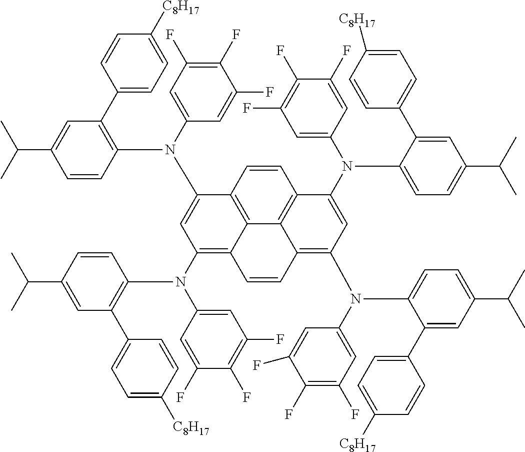 | 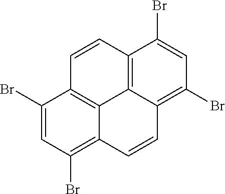 [SMILES: Brc1cc(Br)c2ccc3c(Br)cc(Br)c4ccc1c2c43] | 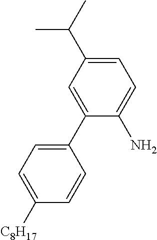 | 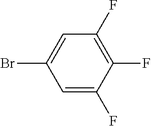 |
| Com- pound 23 | 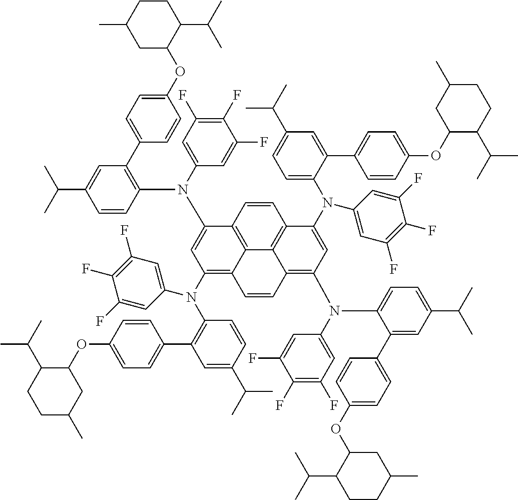 [SMILES: CC1CCC(C(C)C)C(Oc2ccc(-c3cc(C(C)C)ccc3N(c3cc(F)c(F)c(F)c3)c3cc(N(c4cc(F)c(F)c(F)c4)c4ccc(C(C)C)cc4-c4ccc(OC5CC(C)CCC5C(C)C)cc4)c4ccc5c(N(c6cc(F)c(F)c(F)c6)c6ccc(C(C)C)cc6-c6ccc(OC7CC(C)CCC7C(C)C)cc6)cc(N(c6cc(F)c(F)c(F)c6)c6ccc(C(C)C)cc6-c6ccc(OC7CC(C)CCC7C(C)C)cc6)c6ccc3c4c65)cc2)C1] | 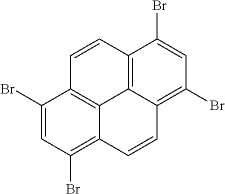 [SMILES: Brc1cc(Br)c2ccc3c(Br)cc(Br)c4ccc1c2c43] | 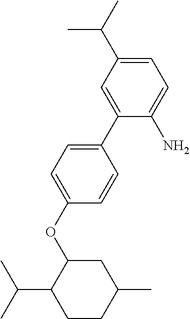 [SMILES: CC1CCC(C(C)C)C(Oc2ccc(-c3cc(C(C)C)ccc3N)cc2)C1] | 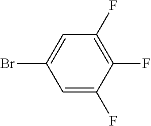 |
| Com- pound 24 | 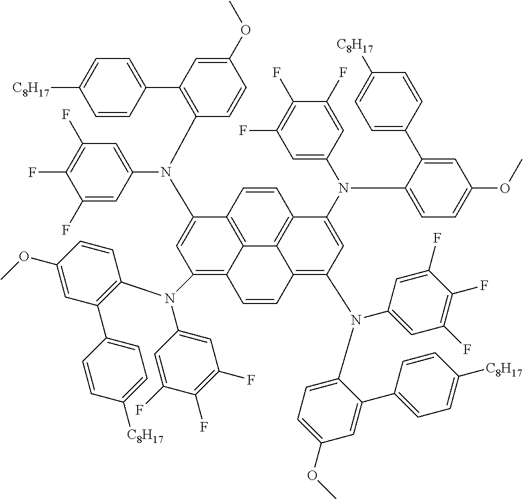 | 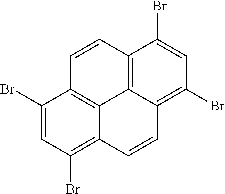 [SMILES: Brc1cc(Br)c2ccc3c(Br)cc(Br)c4ccc1c2c43] | 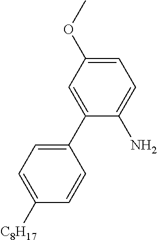 | 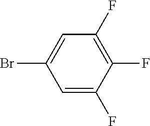 |
| Com- pound 25 | 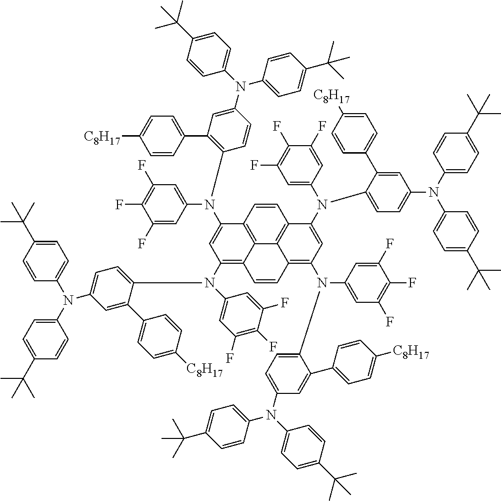 | 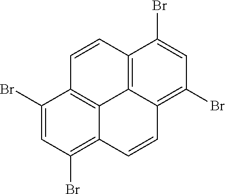 [SMILES: Brc1cc(Br)c2ccc3c(Br)cc(Br)c4ccc1c2c43] | 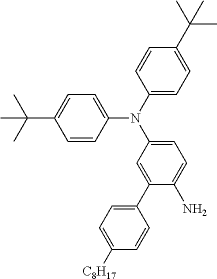 | 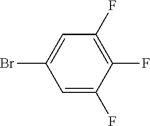 |
| Com- pound 26 | 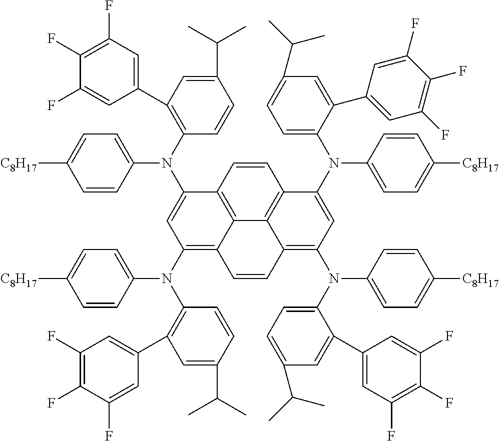 | 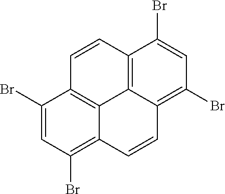 [SMILES: Brc1cc(Br)c2ccc3c(Br)cc(Br)c4ccc1c2c43] | 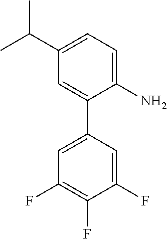 [SMILES: CC(C)c1ccc(N)c(-c2cc(F)c(F)c(F)c2)c1] | 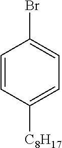 |

12. Synthesis of Polymer P1, where x:y=1:10

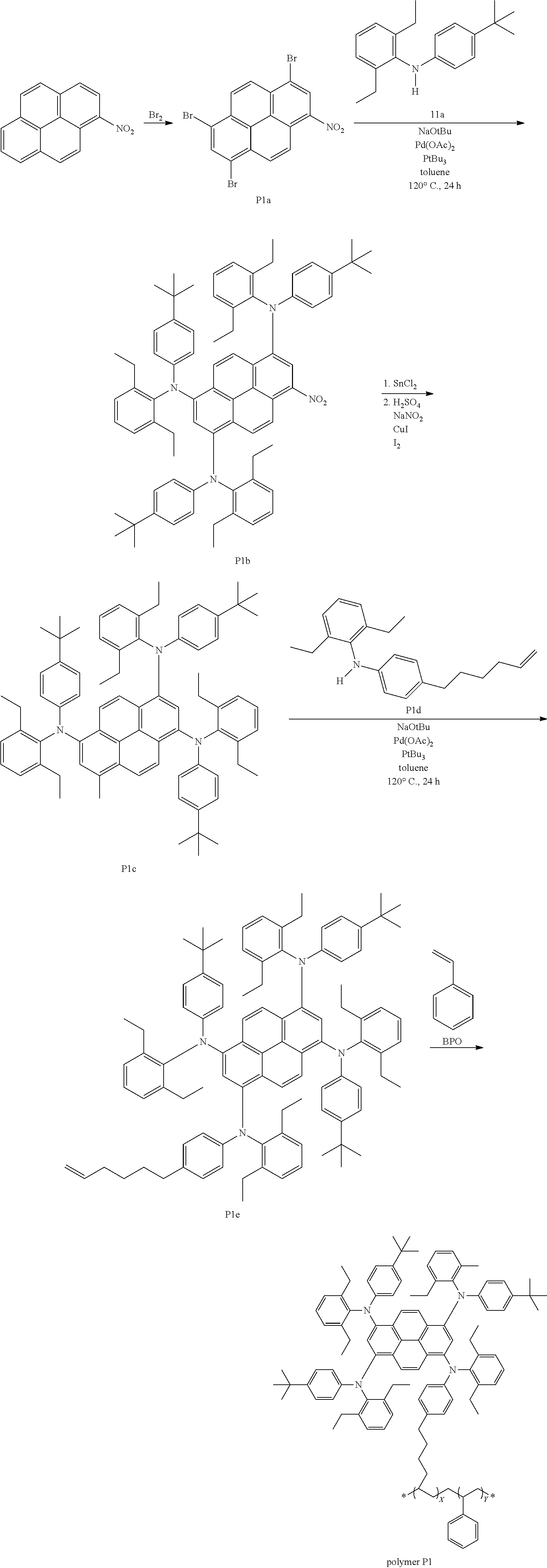

1-Nitropyrene (10 g, 40.44 mmol) and 500 mL of DCM were added to a 1000 mL dry-clean three-necked flask, then bromine (9.69 g, 121.3 mmol) was added under N₂ atmosphere in the dark. The mixture was reacted for 12 h in the dark. The organics were extracted with dichloromethane and saturated brine three times, the solvent was removed by rotary evaporation, and then the residue was recrystallized with toluene to yield 15 g (yield: 76.2%) of intermediate P1a (solid powder).

Intermediate P1a (15 g, 31.05 mmol), intermediate 11a (41.55 g, 139.73 mmol), Pd(OAc)₂ (2.325 mmol), PtBu₃ (3.105 mmol), NaOtBu (26.85 g, 279.45 mmol), and 1 L of toluene were added to a 2000 mL dry-clean three-necked flask under N₂ atmosphere. The mixture was heated to 120° C., refluxed for 24 h, then cooled down to room temperature. The mixture was extracted with dichloromethane and saturated brine three times, the combined organic phase was concentrated, and the resulting crude was purified by short silica gel column chromatography (eluent: ethyl acetate:n-hexane=1:20). Then the solvent was removed to yield 24 g (yield: 71.2%) of intermediate P1b.

Intermediate P1b (24 g, 22.11 mmol), SnCl₂ (20.89 g, 110.54 mmol), and 1.5 L of ethanol absolute were added to a 2000 mL dry-clean three-necked flask under N₂ atmosphere, the mixture was heated to 70° C. and reacted for 1 h. The mixture was poured into the ice water and sodium bicarbonate was added to make the solution slightly alkaline. After the filtration, the solid was vacuum-dried, then dissolved in 500 mL of ethanol absolute. After cooling down to −5° C., 200 mL H₂SO₄ solution of NaNO2 (2.42 g, 28.4 mmol) was slowly added to the above mixture with stirring, then a mixture of CuI (0.54 g, 2.84 mmol) and I₂ (3.6 g, 28.4 mmol) was added slowly in batches. After the filtration and drying, 5 g of intermediate Plc was obtained (yield: 19.4%).

Intermediate Plc (5 g, 4.25 mmol), intermediate P1d (2.1 g, 6.5 mmol), Pd(OAc)₂ (0.075 g), PtBu₃ (0.1 g), NaOtBu (1.25 g, 12.75 mmol), and toluene (500 mL) were added to a 1000 mL dry-clean three-necked flask under N₂ atmosphere. The mixture was heated to 120° C., refluxed for 24 h, then cooled down to room temperature. After extracting with dichloromethane and saturated brine three times, the combined organic phase was concentrated and passed through a short silica gel column (eluent: ethyl acetate:n-hexane=1:20), then the solvent was removed to yield 3.5 g (yield: 61.2%) of intermediate P1e.

Intermediate P1e (3.5 g, 2.6 mmol), styrene (2.70 g, 26 mmol), BPO (0.0624 g, 0.26 mmol), and 100 mL of DCM were added to a 250 mL dry-clean three-necked flask under N₂ atmosphere, then stirred. After being irradiated with UV for 12 h, the monomer was removed by dialysis, then the result was dried to yield 1.56 g (yield: 25.1%) of polymer P1.

The structures of E1, E2, and E3 as the green dopants are as follows, where E1 was purchased from Shanghai Macklin Biochemical Technology Co., Ltd.; E2 was synthesized with reference to Chinese Patent Application CN202211429395.8; E3 was synthesized with reference to International Application Publication No. WO2024/104383A1.

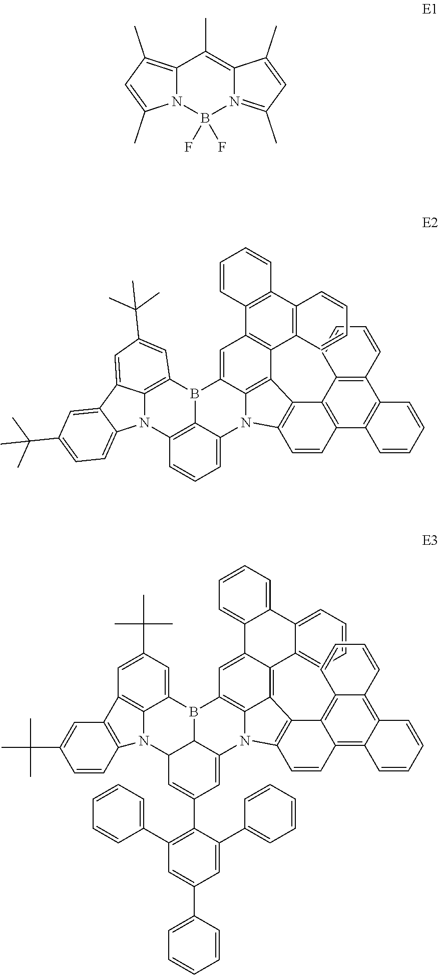

The structure of E4 as a red dopant is as follows, where E4 was synthesized with reference to Chuluo Yang, et. al., Adv. Mater, 2022, 2201442.

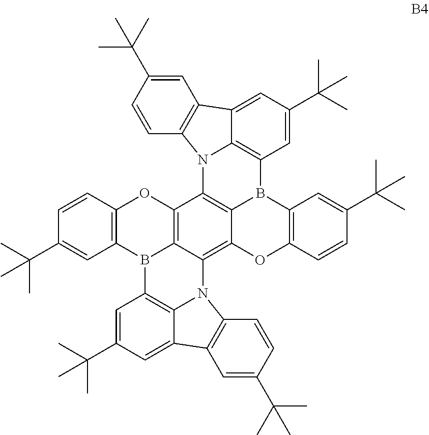 [SMILES: CC(C)(C)c1ccc2c(c1)B1c3c(c4c5c(c3-n3c6ccc(C(C)(C)C)cc6c6cc(C(C)(C)C)cc1c63)Oc1ccc(C(C)(C)C)cc1B5c1cc(C(C)(C)C)cc3c5cc(C(C)(C)C)ccc5n-4c13)O2]

### Example 2: Energy Structures of the Compounds

The energy level of the organic material can be calculated by quantum computation, for example, using TD-DFT (time-dependent density functional theory) by Gaussian 09W (Gaussian Inc.), the specific simulation methods of which can be found in WO2011141110. Firstly, the molecular geometry is optimized by density functional theory “Ground State/DFT/Default Spin/B3LYP” and the basis set “6-31G (d)” (Charge 0/Spin Singlet), then the energy structure of organic molecules is calculated by TD-DFT (time-dependent density functional theory) “TD-SCF/DFT/Default Spin/B3PW91” and the basis set “6-31G (d)” (Charge 0/Spin Singlet). The HOMO and LUMO energy levels are calculated using the following calibration formula, where S1 and T1 are used directly.

HOMO(eV)=((HOMO(G)×27.212)-0.9899)/1.1206

LUMO(eV)=((LUMO(G)×27.212)-2.0041)/1.385

Where HOMO(G) and LUMO(G) are the direct calculation results of Gaussian 09W, in units of Hartree. The results are shown in Table 1 below:

TABLE 1

| | HOMO | LUMO | | | |
|---|---|---|---|---|---|
| | Corr. | Corr. | ΔHOMO | ΔLUMO | |
| Materials | [eV] | [eV] | Corr. [eV] | Corr. [eV] | f(S1) |
| Compound 1 | −4.97 | −2.66 | 0.51 | 0.55 | 0.6113 |
| Compound 2 | −4.87 | −2.61 | 0.69 | 0.55 | 0.6979 |
| Compound 3 | −5.08 | −2.55 | 0.31 | 0.58 | 0.0716 |
| Compound 4 | −4.85 | −2.61 | 0.58 | 0.60 | 0.6376 |
| Compound 5 | −4.97 | −2.70 | 0.33 | 0.63 | 0.6123 |
| Compound 6 | −4.83 | −2.57 | 0.56 | 0.57 | 0.7243 |
| Compound 7 | −4.78 | −2.52 | 0.60 | 0.56 | 0.6819 |
| Compound 8 | −4.73 | −2.51 | 0.68 | 0.52 | 0.6972 |
| Compound 9 | −4.67 | −2.46 | 0.72 | 0.53 | 0.6641 |
| Compound | −4.73 | −2.49 | 0.65 | 0.54 | 0.6695 |
| 10 | | | | | |
| Compound | −4.86 | −2.61 | 0.56 | 0.62 | 0.6107 |
| 11 | | | | | |
| Compound | −4.79 | −2.54 | 0.65 | 0.55 | 0.6725 |
| 12 | | | | | |
| Compound | −5.02 | −2.74 | 0.41 | 0.52 | 0.6428 |
| 13 | | | | | |
| Compound | −4.89 | 2.65 | 0.54 | 0.43 | 0.6965 |
| 14 | | | | | |
| Compound | −5.34 | −2.94 | 0.63 | 0.51 | 0.8657 |
| 15 | | | | | |
| Compound | −5.18 | −2.88 | 0.41 | 0.60 | 0.5838 |
| 16 | | | | | |
| Compound | −5.22 | −2.77 | 0.42 | 0.61 | 0.5743 |
| 17 | | | | | |
| Compound | −5.44 | −2.78 | 0.84 | 0.05 | 0.1662 |
| 18 | | | | | |
| Compound | −5.20 | −2.68 | 0.67 | 0.06 | 0.1708 |
| 19 | | | | | |
| Compound | −5.12 | −2.79 | 0.36 | 0.63 | 0.5760 |
| 20 | | | | | |
| Compound | −4.96 | −2.71 | 0.48 | 0.60 | 0.6337 |
| 21 | | | | | |
| Compound | −5.22 | −2.90 | 0.51 | 0.59 | 0.6241 |
| 22 | | | | | |
| Compound | −5.04 | −2.76 | 0.64 | 0.53 | 0.4870 |
| 23 | | | | | |
| Compound | −4.99 | −2.72 | 0.60 | 0.52 | 0.4983 |
| 24 | | | | | |
| Compound | −4.94 | −2.78 | 0.56 | 0.50 | 0.4985 |
| 25 | | | | | |
| Compound | −4.74 | −2.56 | 0.68 | 0.38 | 0.5214 |
| 26 | | | | | |
| Comparative | −5.30 | −2.92 | 0.62 | 0.67 | 0.5984 |
| compound 1 | | | | | |
| Comparative | −4.95 | −2.11 | 0.32 | 0.00 | 0.0853 |
| compound 2 | | | | | |
| E1 | −5.75 | −3.17 | 1.01 | 2.37 | 0.4938 |
| E2 | −5.53 | −3.04 | 0.26 | 0.58 | 0.4805 |
| E3 | −5.47 | −3.01 | 0.28 | 0.56 | 0.4523 |
| E4 | −5.17 | −2.99 | 0.88 | 0.50 | 0.2802 |

### Example 3: Solubility of Compounds

The solubility of the compounds in the toluene were determined as follows:
1. 1000 mg toluene was placed in the transparent glass sample bottle.
2. A certain mass of the compound was added into the toluene, and oscillated until completely dissolved.
3. After the compound was completely dissolved, a certain mass of the compound was further added into the toluene, and oscillated.
4. The above steps were repeated until the compound wasn't dissolved any more.
5. The total mass of the compound added was recorded and the solubility was obtained by calculation.

The solubility of the compound 1-compound 26 and E1-E4 in the toluene are shown in the Table 2 below.

TABLE 2

| | Solubilities(/100 g | |
|---|---|---|
| Materials | toluene) | |
| Compound 1 | 0.1 | g |
| Compound 2 | 1.5 | g |
| Compound 3 | >2 | g |
| Compound 4 | 4.2 | g |
| Compound 5 | >5 | g |
| Compound 6 | 7.7 | g |
| Compound 7 | 4.6 | g |
| Compound 8 | >20 | g |
| Compound 9 | >20 | g |
| Compound 10 | 30.1 | g |
| Compound 11 | 25.9 | g |
| Compound 12 | >3 | g |
| Compound 13 | >2.5 | g |
| Compound 14 | >2 | g |
| Compound 15 | >4 | g |
| Compound 16 | >1.5 | g |
| Compound 17 | >1 | g |
| Compound 18 | >1 | g |
| Compound 19 | >1 | g |
| Compound 20 | >1.5 | g |
| Compound 21 | >3 | g |
| Compound 22 | >15 | g |
| Compound 23 | >10 | g |
| Compound 24 | >10 | g |
| Compound 25 | >10 | g |
| Compound 26 | >10 | g |
| E1 | >1 | g |
| E2 | >0.7 | g |
| E3 | >0.7 | g |
| E4 | >0.7 | g |

### Example 4: Absorption Spectrum, Emission Spectrum and Extinction Coefficients of Compounds (Comparison Between Solution and Film)

The extinction coefficients of the compounds and their absorption and emission spectrums in the solution were determined as follows:
1. The compound with a certain amount of substance concentration in the toluene was prepared in a volumetric flask.
2. The absorption spectrum of the solution was measured by using an ultraviolet-visible spectrophotometer (Puxi T9s) to obtain the absorption peak wavelength.
3. The absorbance of the maximum absorption peak divided by the amount of substance concentration is the molar extinction coefficient of the compound.
4. The emission spectrum of the solution was measured by using a fluorescent spectroscope (Hitachi, F-4700 FL Spectrophotometer) to obtain the emission peak wavelength.

The Absorption and emission spectrums of the compounds in the film were determined as follows:
1. The compound with a certain concentration in the toluene was prepared.
2. The prepared toluene solution was dropped onto a clean glass substrate.
3. Spin-coating, and then heating at the 80° C. for 1 min.
4. The absorption spectrum of the film was measured by using an ultraviolet-visible spectrophotometer (Puxi T9s) to obtain the absorption peak wavelength.
5. The emission spectrum of the film was measured by using a fluorescent spectroscope (Hitachi, F-4700 FL Spectrophotometer) to obtain the emission peak wavelength.

The extinction coefficients of the compounds and their absorption and emission peaks in the solution and film are shown in Table 3.

TABLE 3

| | Absorption | Emission | Molar extinction | | |
|---|---|---|---|---|---|
| | peak of the | peak of the | coefficient of the | Absorption | Emission |
| | toluene | toluene | toluene | peak of the | peak of the |
| Materials | solution | solution | solution/(L · mol⁻¹) | film | film |
| Compound | 473 nm | 493 nm | 1.7 × 10⁴ | 477 nm | 500 nm |
| 1 | | | | | |
| Compound | 475 nm | 490 nm | 6.0 × 10⁴ | 477 nm | 497 nm |
| 2 | | | | | |
| Compound | 477 nm | 495 nm | 3.0 × 10⁴ | 479 nm | 497 nm |
| 4 | | | | | |
| Compound | 479 nm | 498 nm | 5.5 × 10⁴ | 478 nm | 497 nm |
| 6 | | | | | |
| Compound | 477 nm | 497 nm | 5.6 × 10⁴ | 476 nm | 497 nm |
| 7 | | | | | |
| Compound | 484 nm | 501 nm | 5.3 × 10⁴ | 482 nm | 499 nm |
| 10 | | | | | |
| Compound | 482 nm | 500 nm | 5.3 × 10⁴ | 482 nm | 499 nm |
| 11 | | | | | |
| Compound | 466 nm | 493 nm | 3.7 × 10⁴ | 469 nm | 551 nm |
| 16 | | | | | |
| Compound | 439 nm | 462 nm | 1.8 × 10⁴ | 445 nm | 496 nm |
| 17 | | | | | |
| Compound | 465 nm | 488 nm | 2.3 × 10⁴ | 482 nm | 561 nm |
| 20 | | | | | |
| Compound | 469 nm | 495 nm | 4.0 × 10⁴ | 472 nm | 546 nm |
| 21 | | | | | |
| Compound | 473 nm | 494 nm | 5.9 × 10⁴ | 473 nm | 490 nm |
| 22 | | | | | |
| Compound | 476 nm | 493 nm | 6.8 × 10⁴ | 473 nm | 491 nm |
| 23 | | | | | |
| Compound | 475 nm | 496 nm | 7.0 × 10⁴ | 476 nm | 496 nm |
| 24 | | | | | |
| Compound | 492 nm | 520 nm | 5.1 × 10⁴ | 487 nm | 518 nm |
| 25 | | | | | |
| Compound | 485 nm | 504 nm | 5.5 × 10⁴ | 486 nm | 504 nm |
| 26 | | | | | |
| Comparative | 461 nm | 485 nm | 4.3 × 10⁴ | 464 nm | 543 nm |
| compound | | | | | |
| 1 | | | | | |
| E1 | 500 nm | 519 nm | 6.1 × 10⁴ | — | — |
| E2 | 507 nm | 523 nm | 4.8 × 10⁴ | — | — |
| E3 | 512 nm | 530 nm | 6.6 × 10⁴ | — | — |
| E4 | 589 nm | 613 nm | 2.7 × 10⁴ | — | — |

As shown in Table 3, the measured organic compounds H exhibit high molar extinction coefficients.

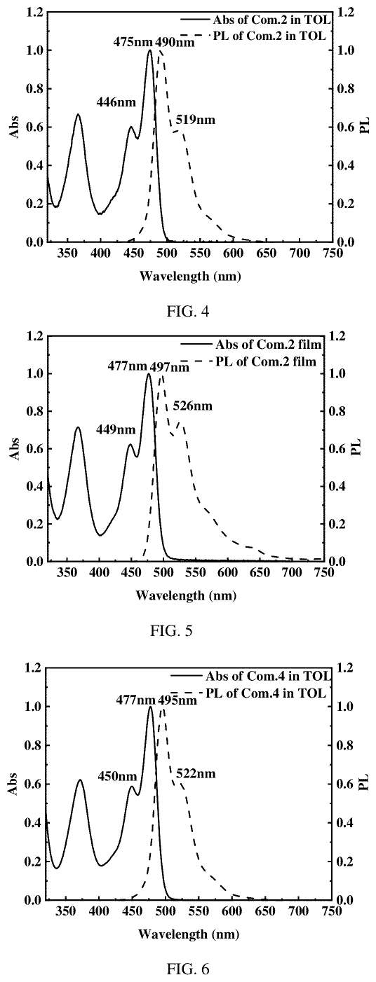

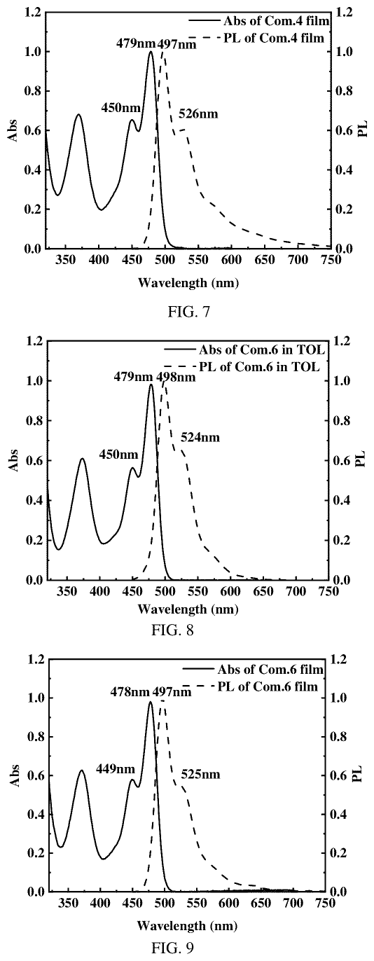

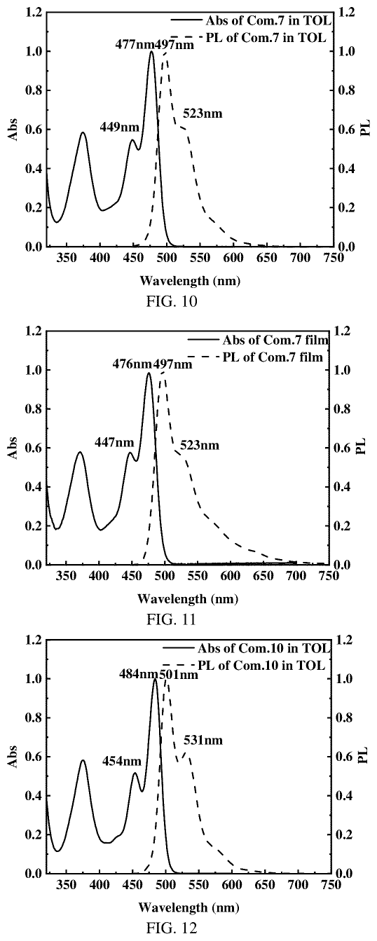

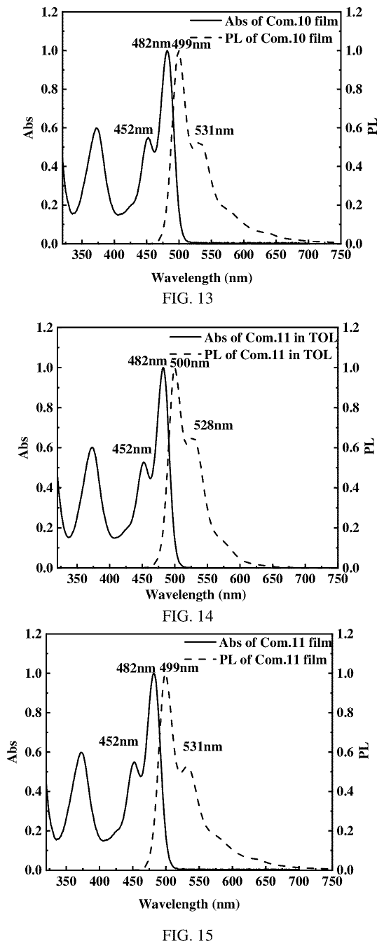

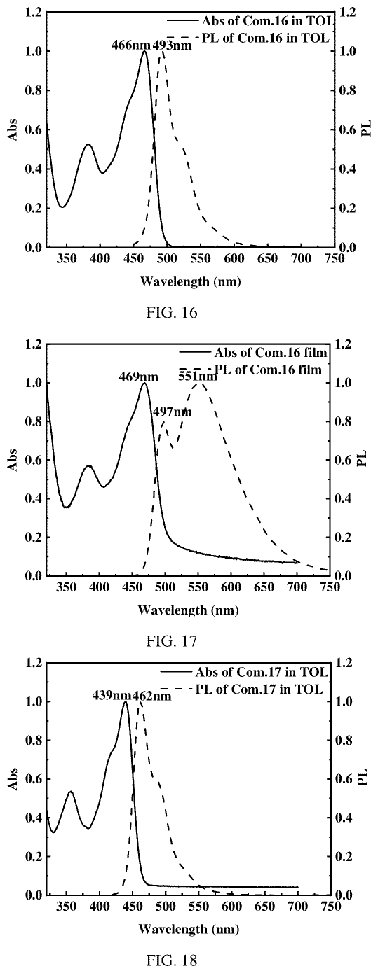

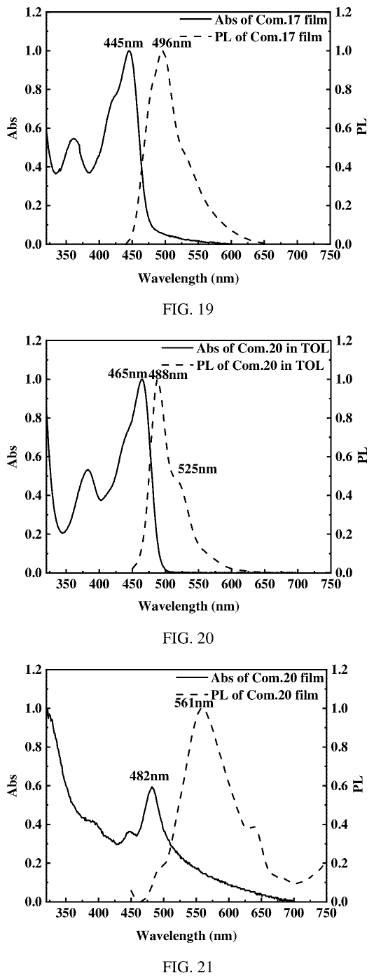

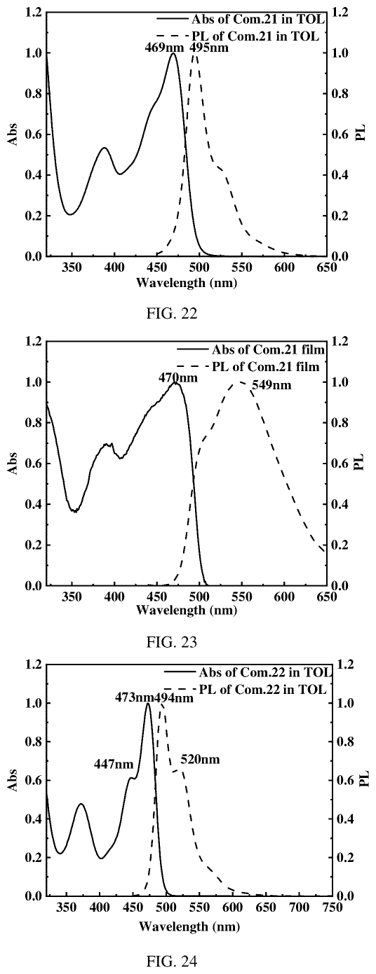

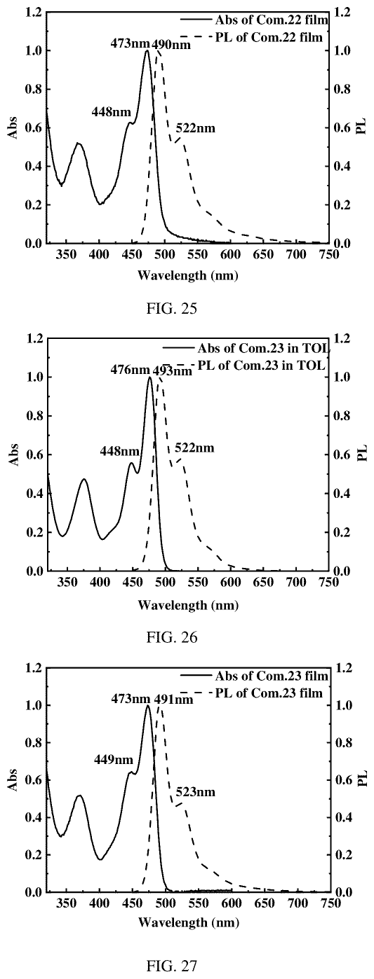

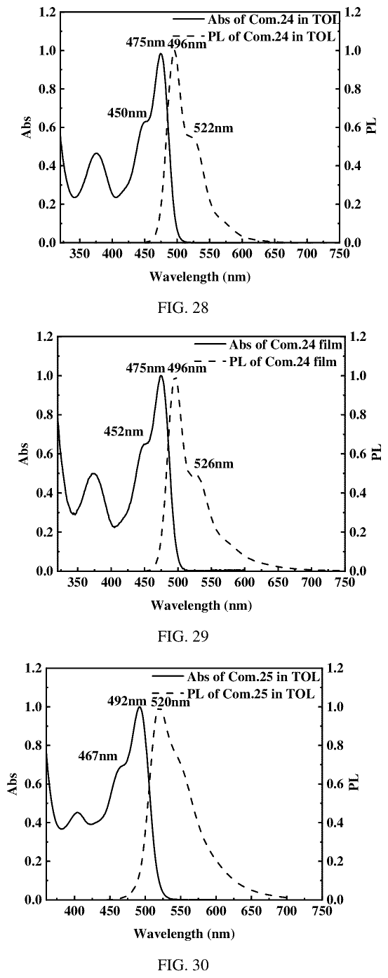

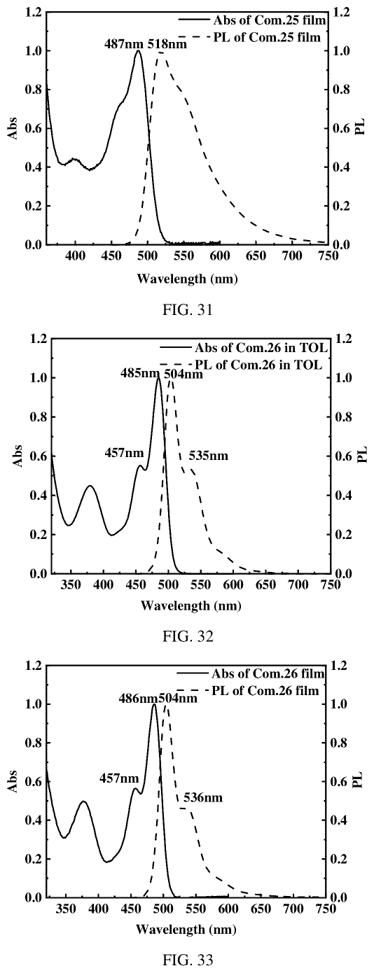

FIG. 2-FIG. 33 show the absorption and emission spectrums of the compounds 1, 2, 4, 6, 7, 10, 11, 16, 17, 20, 21, 22, 23, 24, 25, 26 in the solution and film. It can be seen from these figures that the absorption and emission spectrums in the solution and film of the compound 1 according to formula (Ia-1), and the compounds 2, 4, 6, 7, 10, 11, 22, 23, 24, 25, and 26 according to formula (Ia-3) are very similar, while the red shift of the spectrum in thin film is very small. This is due to the fact that in the aryl amines, the neighboring substitutions linked to the aryl group (here, benzene) effectively prevent molecular stacking in the film. Similarly, the compounds of formulas (Ia-2), (Ia-4), (Ia-5), (Ia-6) also have the same effect.

On the other hand, the spectrums of the compounds 16, 17, 20, 21 in the film have a large red shift relative to the spectrums in the solution, and the emission spectrums become wide due to the molecular stacking effect in the film. Nevertheless, it is still possible for the compounds 16, 17, 20, 21 to avoid stack by combining with other compounds or polymers to form a mixture, maintaining a spectrum similar to that in the solution, and thus suitable for color conversion layers.

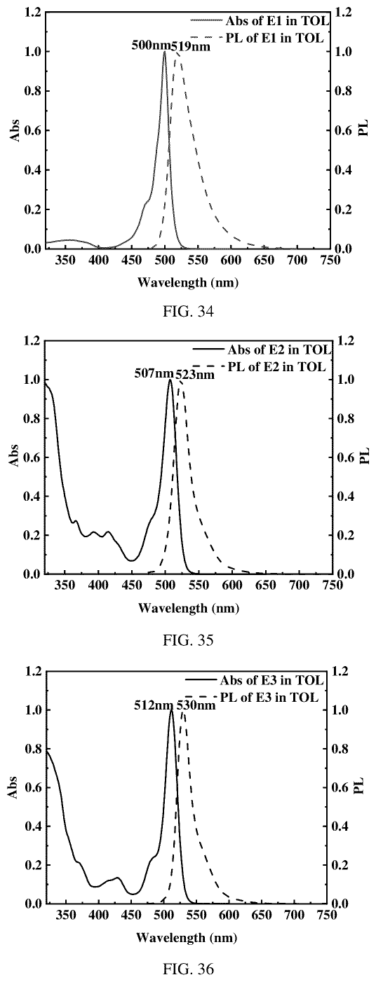

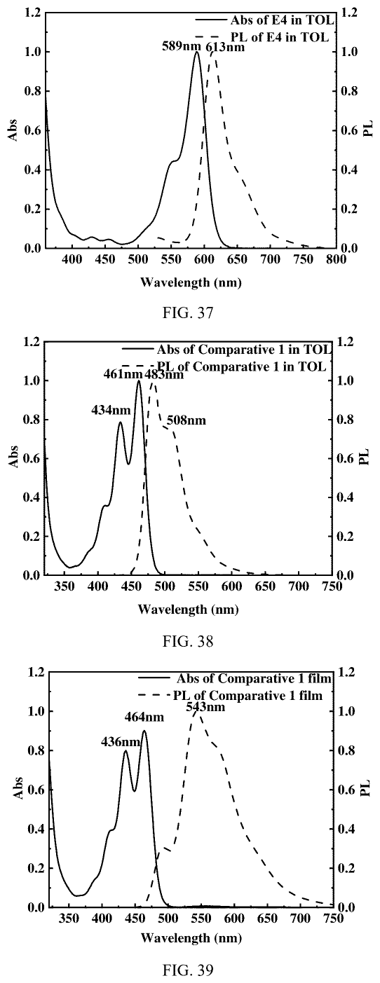

The optical properties of the compounds as described herein (i.e., example 4): the absorption and emission spectrums were respectively measured by the spectrophotometer (Puxi T9s) and the fluorescent spectroscope (Hitachi, F-4700 FL Spectrophotometer). FIG. 34 shows the absorption and emission spectrums of the compound E1 in the toluene. FIG. 35 shows the absorption and emission spectrums of the compound E2 in the toluene. FIG. 36 shows the absorption and emission spectrums of the compound E3 in the toluene. FIG. 37 shows the absorption and emission spectrums of the compound E4 in the toluene. FIG. 38 and FIG. 39 respectively show the absorption and emission spectrums of the comparative compound 1 in the toluene and film. The emission spectrum of comparative compound 1 has a large red shift, which may affect the color purity of the green light, although it can still be used as a green or red host. In contrast, the film spectrums of the compounds 2, 4, 6, 7, 10, 11, 22, 23, 24, 25, 26 have a very small red shift, which are more favorable for preparing the color conversion layers (CCLs) with high color purity.

### Example 5: UV Stability of Compounds

The UV stability of the compounds were tested as follows:

The compound was dissolved in the toluene with a concentration of 1×10⁵ mol/L. 3 mL solution was added to the cuvette with a lid and the lid was screwed on tightly, then the cuvette was placed in the ultraviolet-visible spectrophotometer to test the absorption spectrum, the absorbance of the maximum absorption peak was recorded as the initial value. The cuvette was placed at a distance of 12 cm from the UV LEDs (365 nm & 255 nm), irradiated for a period and tested its absorption spectrum, then continued to be irradiated after the test, and repeated until the absorbance decayed to 80% of its initial value, the time was recorded as t80.

The t80 of each compound is shown in Table 4 below:

TABLE 4

| Materials | Times t80 | Materials | Times t80 |
|---|---|---|---|
| Compound 1 | 127 min | Compound 1 | 277 min |
| Compound 2 | 175 min | Compound 20 | 188 min |
| Compound 4 | 195 min | Compound 22 | 377 min |
| Compound 6 | 215 min | Compound 23 | 405 min |
| Compound 7 | 280 min | Compound 24 | 352 min |
| Compound 10 | 240 min | Compound 26 | 486 min |
| Compound 11 | 257 min | Comparative | 92 min |
| | | compound 2 | |
| Compound 16 | 227 min | | |

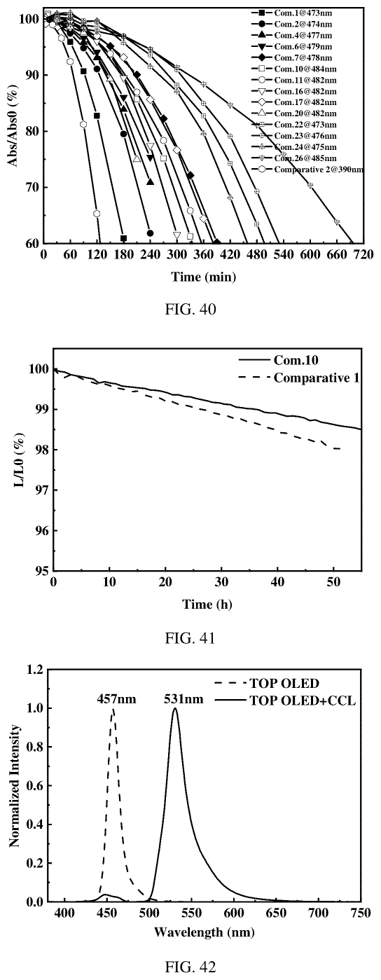

FIG. 40 shows the absorption decay of the compound 1, compound 2, compound 4, compound 6, compound 7, compound 10, compound 11, compound 16, compound 17, compound 20, compound 22, compound 23, compound 24, compound 26, and comparative compound 2 after UV irradiation in the toluene.

As shown in Table 4 and FIG. 40: 1) compared with the comparative compound 2, the UV stability of the organic compound H as described herein is greatly improved; 2) compounds 16, 17 and 20 having similar structures, the stability of the fluorinated compound 16 and the fluorinated compound 17 is also greatly improved than that of the unfluorinated compound 20.

### Example 6: Blue Light Stability of the Film

The blue light stability of the films of the compound 10 and the comparative compound 1 were tested as follows:

A film about 800 nm was evaporated, encapsulated with a glass cover plate, then placed at 2.5 cm above the blue LED (460 nm, 3000 cd/m²) to test the luminance value with a luminance meter (Fstar, CS-2000A). The luminance value of the first test was recorded as the initial value, and the luminance was tested by irradiating for a period of time to obtain the luminance decay curve, see FIG. 41. As seen in FIG. 41, the film of the compound 10 is obviously improved UV stability compared with that of the comparative compound 1.

### Example 7: Preparation of the Color Conversion Layer (CCL)

7.1 Evaporated films: compound 10 and light-emitting material E1, E2 or E3 were respectively placed in a crucible, and the crucibles were put into a thermal evaporation equipment. The cavity was vacuumed with the vacuum degree reaching 1×10⁻⁴ Pa, and then the crucibles began to be heated. The two organic compounds were deposited on the glass substrate by thermal evaporation. After the film reaching the target thickness, the crucibles were stopped heating, and then cooled down to 80° C. The cavity was filled with nitrogen to atmospheric pressure, and opened to obtain the evaporated CCL film.

### 7.2 Solution-Processed Films:

48 mg of compound 10 was dissolved in 1 mL of toluene, stirred for 30 min, and then 2 mg of a light-emitting material E1, E2 or E3 was dissolved in the above solution. After stirred for 30 min, the solution was dropped on a glass substrate, spun-coated and heated at 80° C. for 5 min in order to obtain the CCL film.

### 7.3 Resin Films:

48 mg compound 10 was dissolved in the 1 mL resin solution, stirred for 30 min, and then 2 mg light-emitting material E1, E2 or E3 was dissolved in the above solution. After stirred for 30 min, the solution was dropped on a glass substrate, spun-coated, then UV cured in order to obtain a CCL film.

CCL based on other organic compounds can be prepared in the same way according to 7.1, 7.2, or 7.3.

### Example 8: Results of OLEDs or LEDs+CCL Films

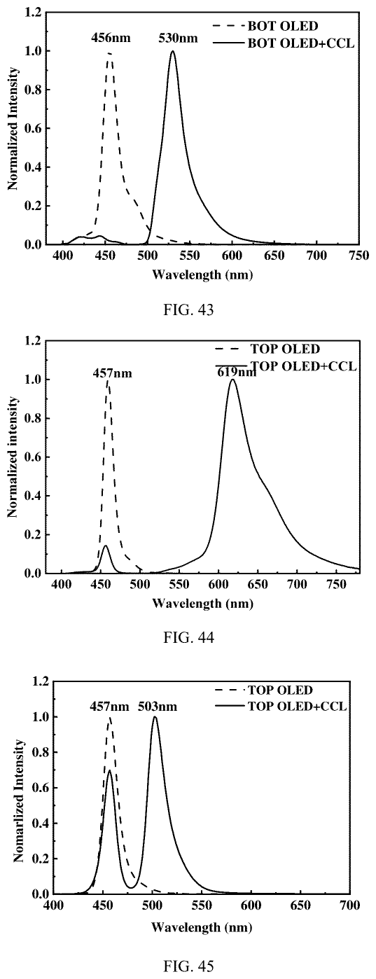

1. Blue top-emitting OLED+resin film of CCL: a resin film of CCL (E3 or E4 as an emitter) with the thickness of about 4 μm was prepared according the above-mentioned 7.3 on the light extraction surface of the blue top-emitting OLED.
2. Blue bottom-emitting OLED+resin film of CCL: a resin film of CCL (E3 as an emitter) with the thickness of about 4 μm was prepared according the above-mentioned 7.3 on the light extraction surface of the blue bottom-emitting OLED.
3. The spectrums of the blue top-emitting OLED, blue top-emitting OLED+resin film of the CCL, blue bottom-emitting OLED, and blue bottom-emitting OLED+resin film of the CCL were tested by the luminance meter (Fstar, CS-2000A). FIG. 42 shows the spectrums of the blue top-emitting OLED and that combined with the resin film of the CCL (E3 as an emitter). FIG. 43 shows the spectrums of the blue bottom-emitting OLED and that combined with the resin film of the CCL (E3 as an emitter). FIG. 44 shows the spectrum of the blue top-emitting OLED and that combined with the resin film of the CCL (E4 as an emitter). As shown in FIG. 42 and FIG. 43, the resin film of the CCL as described herein can basically absorb all the blue light of the OLED in top-emission and bottom-emission devices, and convert the blue light into narrow-spectrum green light. As shown in FIG. 44, the resin film of the CCL as described herein can basically absorb all the blue light of the OLED in top-emission devices, and convert the blue light into narrow-spectrum red light.

### Example 9: OLED with CCL (Top-Emitting OLED+Evaporated Film of the CCL)

1. Blue top-emitting OLED+evaporated film of the CCL: the evaporated film of the CCL (E2 as an emitter) with the thickness of about 500 nm was prepared according the above-mentioned 7.1 on the light extraction surface of the blue top-emitting OLED.
2. The spectrums of the blue top-emitting OLED and blue top-emitting OLED+evaporated film of the CCL were tested by the luminance meter (Fstar, CS-2000A). As shown in FIG. 45, the blue top-emitting OLED+evaporated film of the CCL can obtain narrow-spectrum green light, but there is still some blue light transmittnig due to the CCL film is thin. This problem can be eliminated by increasing the thickness of the CCL film.

The technical features of the above-described embodiments can be combined in any ways. For the sake of brevity, not all possible combinations of the technical features of the above-described embodiments have been described. However, as long as there are no contradictions in the combination of these technical features, they should be considered to be within the scope of this specification.

What described above are several embodiments of the present disclosure, and they are specific and in detail, but not intended to limit the scope of the present disclosure. It will be understood that improvements can be made without departing from the concept of the present disclosure, and all these modifications and improvements are within the scope of the present disclosure. The scope of the present disclosure shall be subject to the appended claims.

What is claimed is:

1. A formulation, comprising an organic compound H of formula (I), an emitter E, wherein 1) the emission spectrum of the organic compound H is on the short wavelength side of the absorption spectrum of the emitter E, and at least partially overlaps with the absorption spectrum of the emitter E; 2) the FWHM of the emission spectrum of the emitter E≤55 nm; 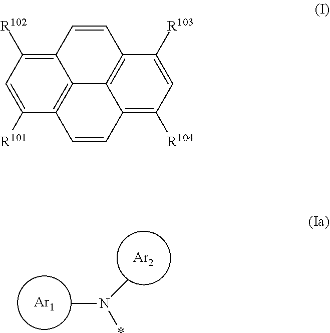
wherein: each of R¹⁰¹ to R¹⁰⁴ is independently selected from —H, -D, a C₁-C₂₀ linear alkyl group, a C₁-C₂₀ linear alkenyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents or any combination thereof,
wherein, at least one of R¹⁰¹-R¹⁰⁴ is a formula (Ia), each of Ar¹ and Ar² is a substituted/unsubstituted aromatic or heteroaromatic group containing 5 to 24 ring atoms, each * independently represents an attachment site connecting a pyrene.

2. The formulation according to claim 1, wherein at least one of R¹⁰¹-R¹⁰⁴ is selected from one of formulas (Ia-1)-(Ia-4): 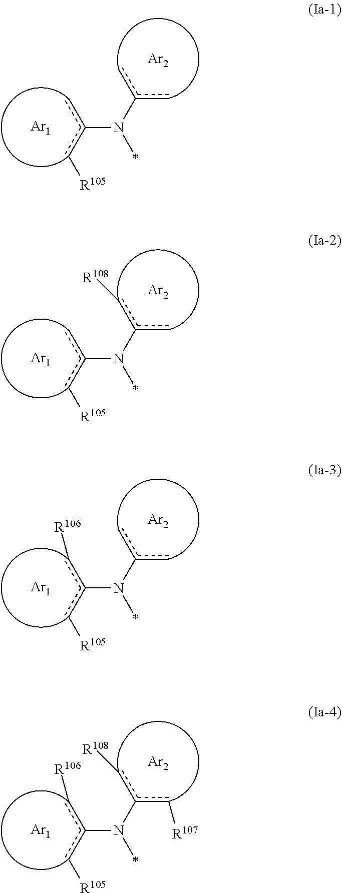
wherein: each of R¹⁰⁵ to R¹⁰⁸ is a substituent, and at each occurrence is independently selected from a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, or any combination thereof.

3. The formulation according to claim 1, wherein the emitter E comprises a structural unit of formula (1), formula (2), formula (3), or formula (4): 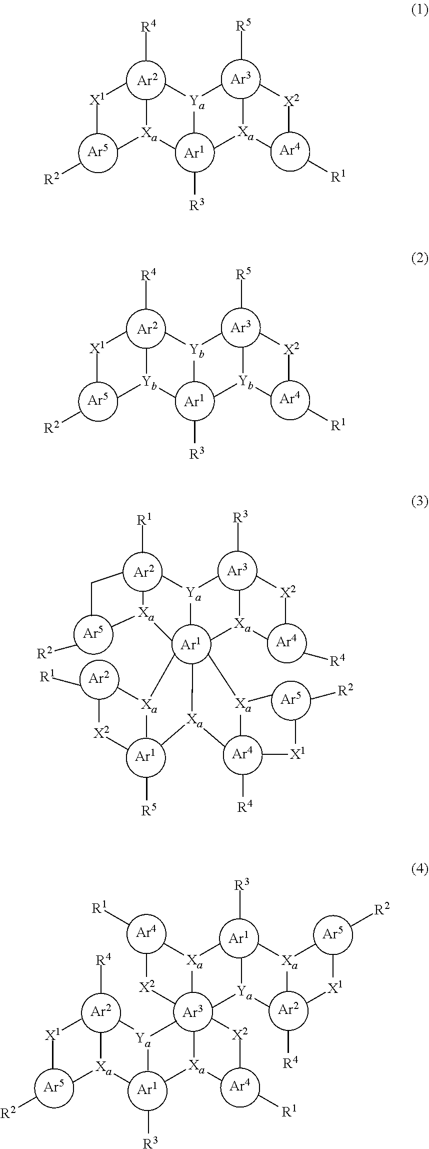
wherein: each of Ar¹ to Ar³ is independently selected from an aromatic group or a heteroaromatic group containing 5 to 24 ring atoms;
each of Ar⁴ and Ar⁵ is independently selected from null, an aromatic group or a heteroaromatic group containing 5 to 24 ring atoms;
when neither Ar⁴ nor Ar⁵ is null, each of Xₐ and X_{b} is independently selected from N, C(R⁶), or Si(R⁶); each of Yₐ and Y_{b} is independently selected from B, P═O, C(R⁶), or Si(R⁶);
when Ar⁴ and/or Ar⁵ is null, each X_{b} is independently selected from N, C(R⁶), or Si(R⁶); each Yₐ is independently selected from B, P═O, C(R⁶), or Si(R⁶); each of Xₐ and Y_{b} is independently selected from N(R⁶), C(R⁶R⁷), Si(R⁶R⁷), C═O, O, C═N(R⁶), C═C(R⁶R⁷), P(R⁶), P(═O)R⁶, S, S═O, or SO₂;
each of X¹ and X² is independently null or a bridging group;
each of R¹ to R⁷ is independently selected from —H, -D, a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents or any combination thereof, wherein one or more R¹-R⁷ form a monocyclic or polycyclic aliphatic or aromatic ring system with each other and/or with the rings bonded thereto.

4. The formulation according to claim 2, wherein the emitter E comprises a structural unit of formula (1), formula (2), formula (3), or formula (4): 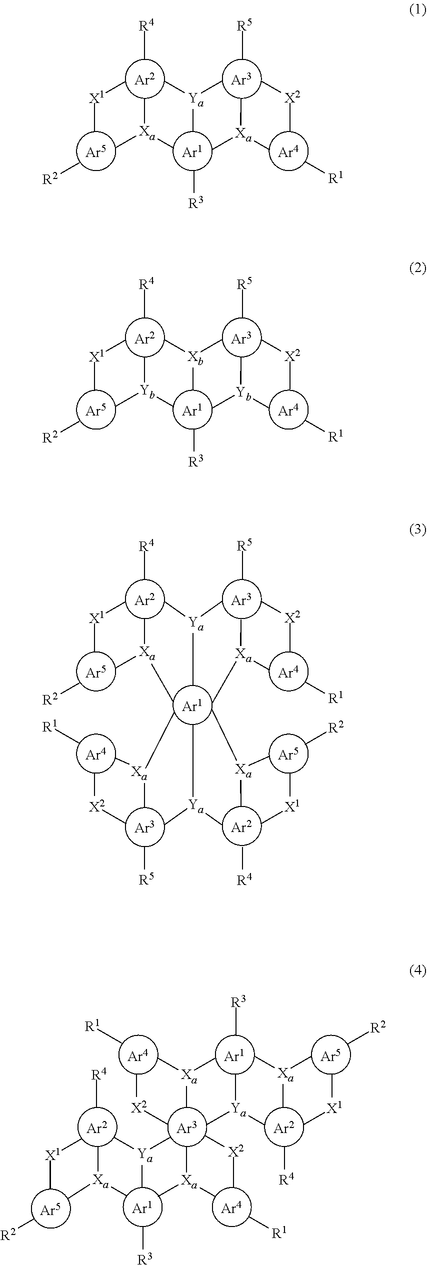
wherein: each of Ar¹ to Ar³ is independently selected from an aromatic group or a heteroaromatic group containing 5 to 24 ring atoms;
each of Ar⁴ and Ar⁵ is independently selected from null an aromatic group or a heteroaromatic group containing 5 to 24 ring atoms;
when neither Ar⁴ nor Ar⁵ is null, each of Xₐ and X_{b} is independently selected from N, C(R⁶), or Si(R⁶); each of Yₐ and Y_{b} is independently selected from B, P═O, C(R⁶), or Si(R⁶);
when Ar⁴ and/or Ar⁵ is null, each X_{b} is independently selected from N, C(R⁶), or Si(R⁶); each Yₐ is independently selected from B, P═O, C(R⁶), or Si(R⁶); each of Xₐ and Y_{b} is independently selected from N(R⁶), C(R⁶R⁷), Si(R⁶R⁷), C═O, O, C═N(R⁶), C═C(R⁶R⁷), P(R⁶), P(═O)R⁶, S, S═O, or SO₂;
each of X¹ and X² is independently null or a bridging group;
each of R¹ to R⁷ is independently selected from —H, -D, a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents or any combination thereof, wherein one or more R¹-R⁷ form a monocyclic or polycyclic aliphatic or aromatic ring system with each other and/or with the rings bonded thereto.

5. The formulation according to claim 1, wherein the emitter E comprises a structural unit of one of formulas (1a)-(1e), (2a)-(2e), (3a)-(3d), or (4a)-(4d₂):

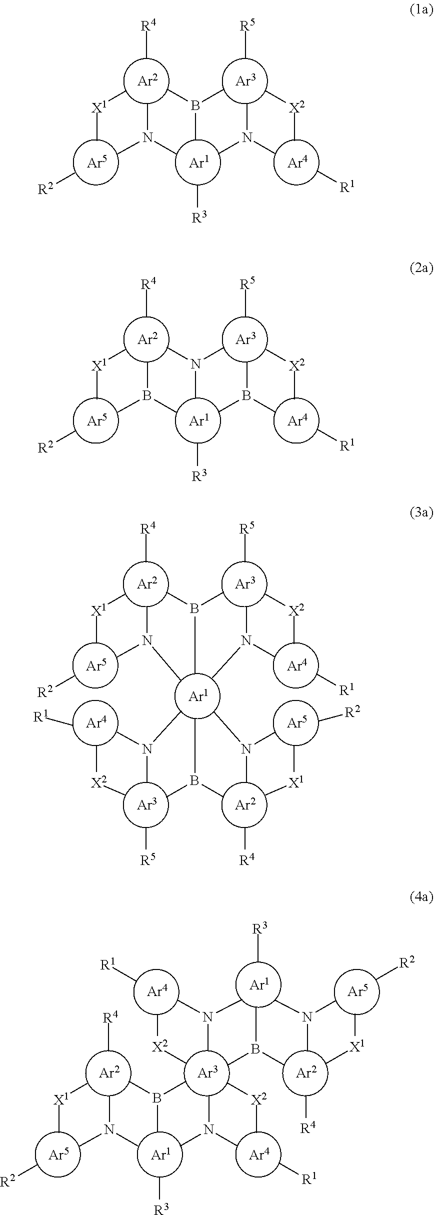

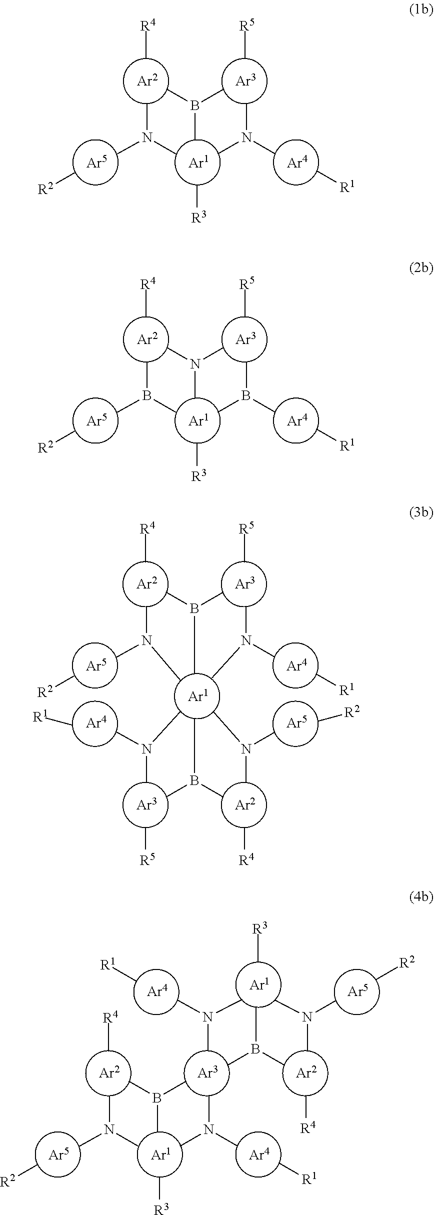

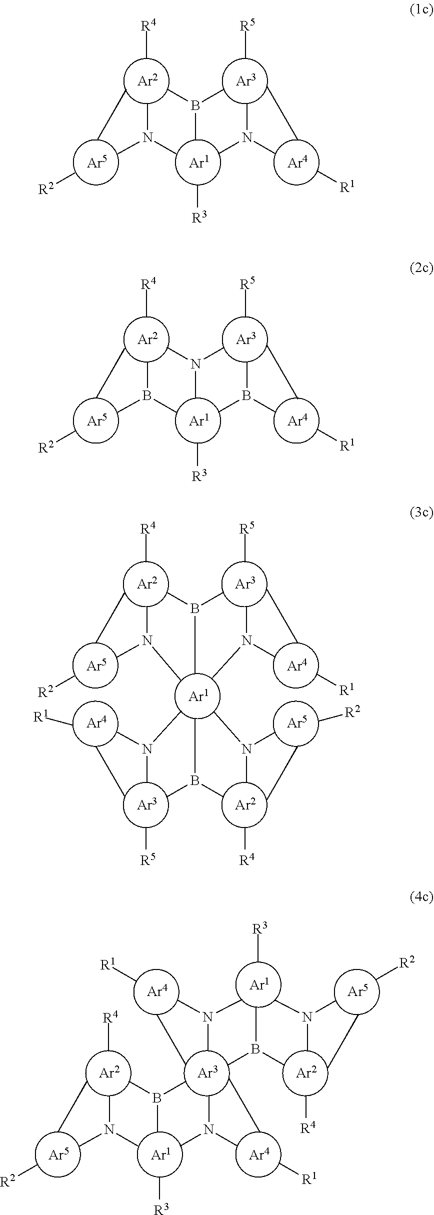

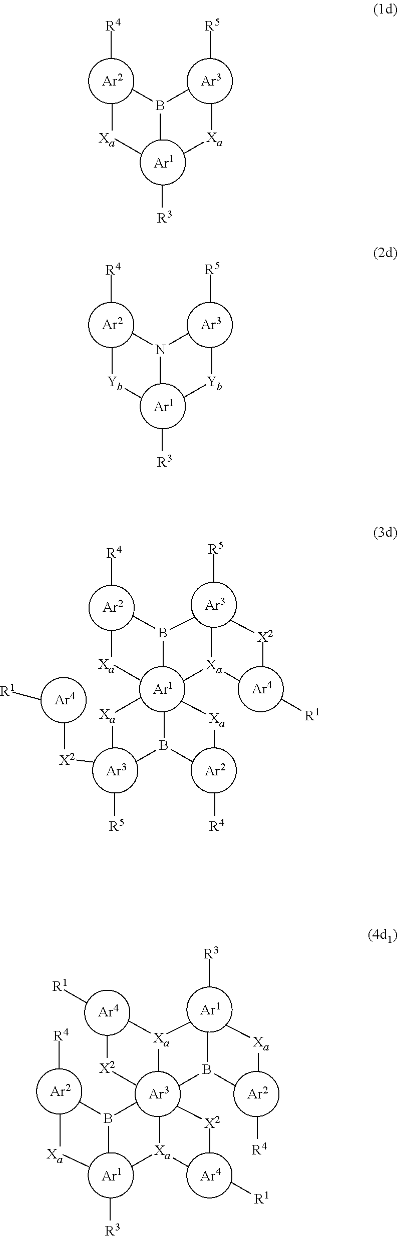

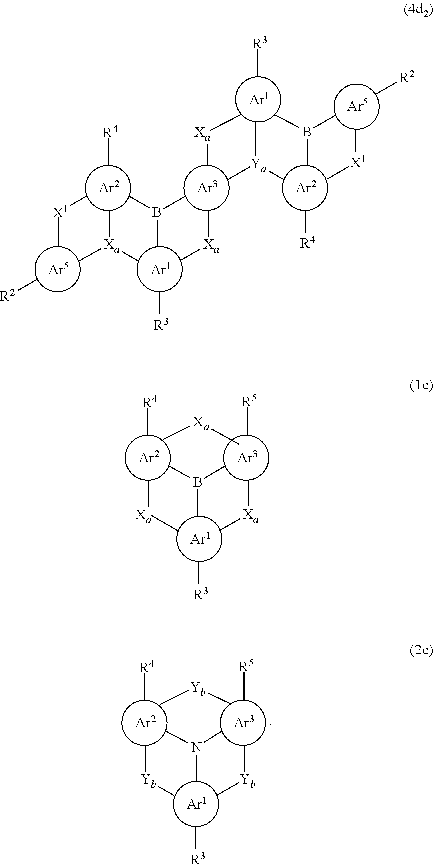

6. The formulation according to claim 1, wherein each of Ar₁, Ar₂, Ar¹, Ar², Ar³, Ar⁴, and Ar⁵ is independently selected from one of the following structural formulas or any combination thereof:

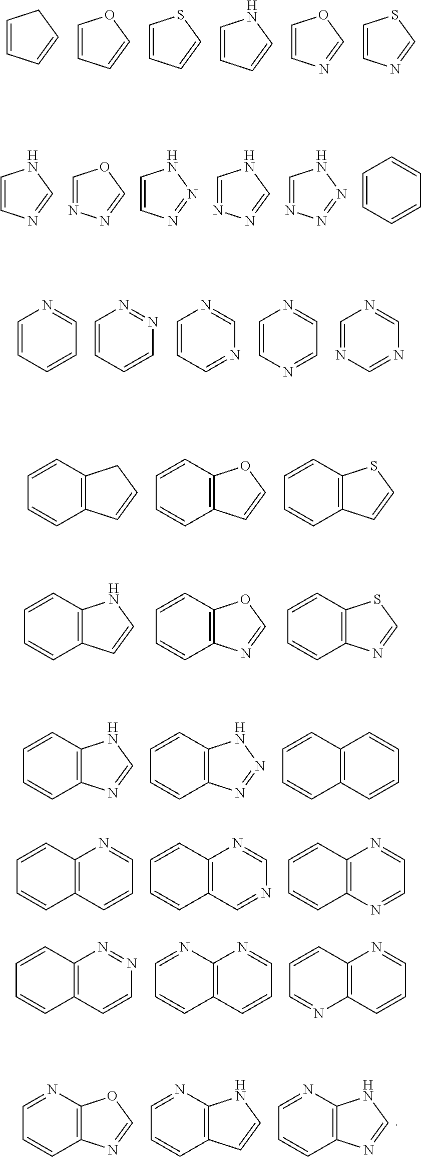

7. The formulation according to claim 2, wherein each of Ar¹, Ar², Ar¹, Ar², Ar³, Ar⁴, and Ar⁵ is independently selected from one of the following structural formulas or any combination thereof:

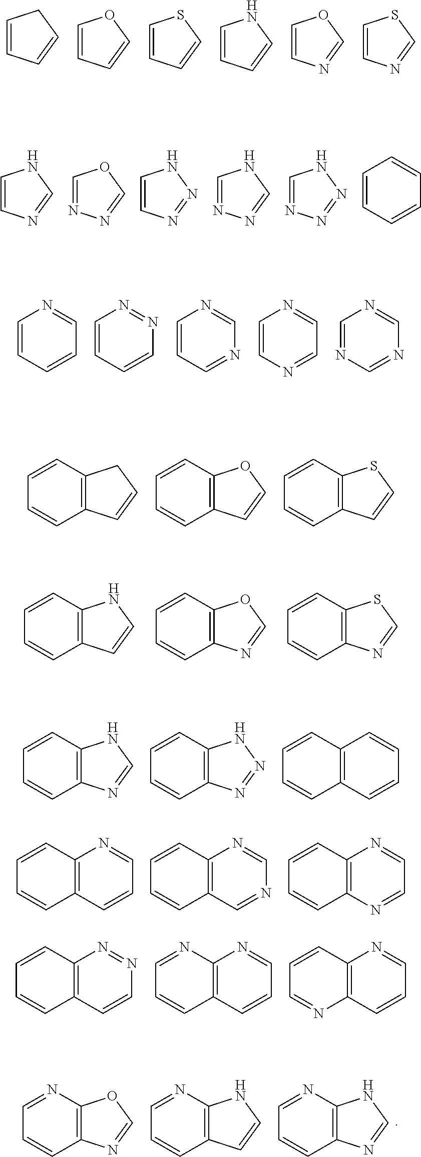

8. The formulation according to claim 3, wherein each of Ar¹, Ar², Ar¹, Ar², Ar³, Ar⁴, and Ar⁵ is independently selected from one of the following structural formulas or any combination thereof:

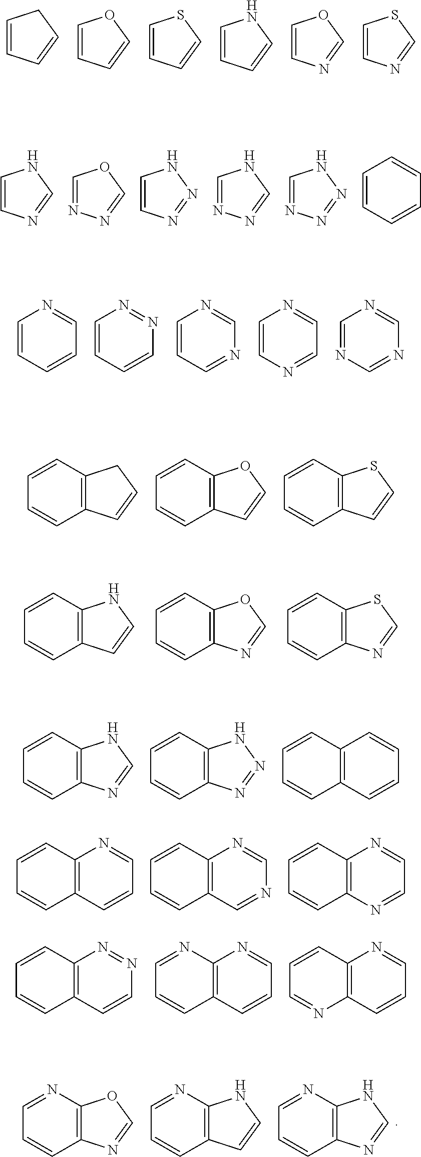

9. The formulation according to claim 4, wherein each of Ar¹, Ar², Ar¹, Ar², Ar³, Ar⁴, and Ar⁵ is independently selected from one of the following structural formulas or any combination thereof:

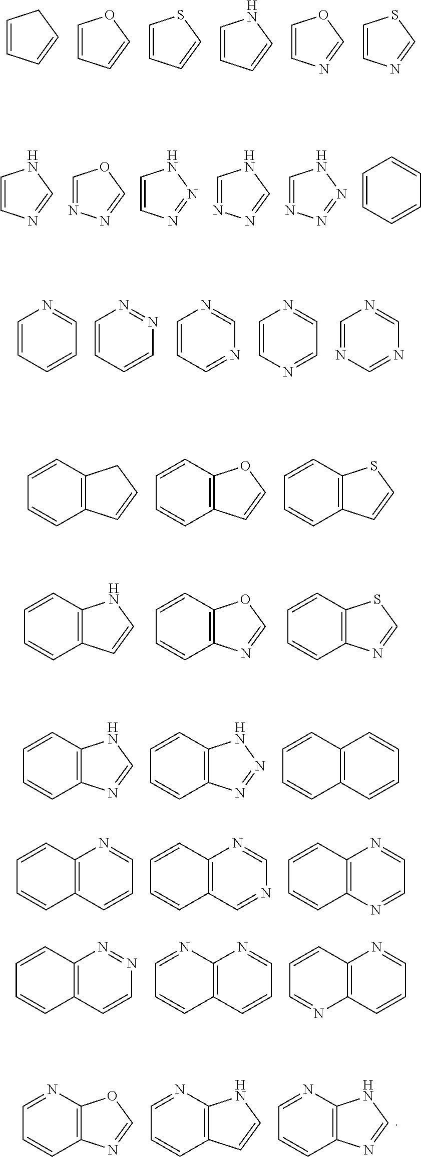

10. The formulation according to claim 5, wherein each of Ar¹, Ar², Ar¹, Ar², Ar³, Ar⁴, and Ar⁵ is independently selected from one of the following structural formulas or any combination thereof:

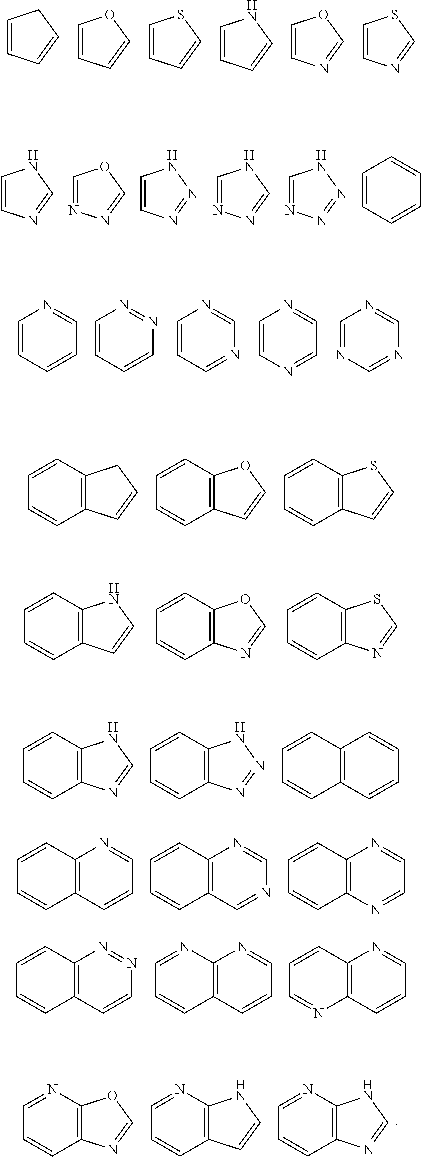

11. The formulation according to claim 1, wherein the organic compound H is polymerized to form a polymer.

12. The formulation according to claim 1, wherein the emitter E is a compound comprising the following structural formula: 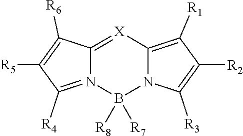
wherein: X is CR₉ or N;
each of R₁ to R₉ is independently selected from a hydrogen, an alkyl group, a cycloalkyl group, a heterocyclic group, an alkenyl group, a cycloalkenyl group, an alkynyl group, a hydroxyl group, a sulfhydryl group, an alkoxy group, an alkylthio group, an aryl ether group, an aryl thioether group, an aryl group, a heteroaryl group, a halogen, a cyano group, an aldehyde group, a carbonyl group, a carboxyl group, an oxycarboxyl group, a carbamoyl group, an amino group, a nitro group, a silyl group, a siloxanyl group, a boranyl group, or a phosphine oxide group; and R₁-R₉ form a fused ring and an aliphatic ring with the adjacent substituents.

13. The formulation according to claim 1, wherein the formulation further comprises an organic resin and/or a solvent.

14. The formulation according to claim 1, wherein the organic resin is a thermosetting resin or an UV curable resin.

15. The formulation according to claim 1, wherein the mass fraction of the organic resin ranges from 20 wt % to 99 wt %.

16. An organic functional film, comprising the formulation according to claim 1.

17. An optoelectronic device, comprising the formulation according to claim 1.

18. The optoelectronic device according to claim 17, wherein the optoelectronic device is an organic light-emitting device comprising a first electrode, an organic light-emitting layer, a second electrode, a color conversion layer, and an encapsulation layer in sequence from bottom to top, the second electrode is at least partially transparent, the color conversion layer at least partially absorbs the light emitted by the organic light-emitting layer through the second electrode, wherein the color conversion layer comprises the formulation.

19. An optoelectronic device, comprising the organic functional film according to claim 16.

20. The optoelectronic device according to claim 19, wherein the optoelectronic device is an organic light-emitting device comprising a first electrode, an organic light-emitting layer, a second electrode, a color conversion layer, and an encapsulation layer in sequence from bottom to top, the second electrode is at least partially transparent, the color conversion layer at least partially absorbs the light emitted by the organic light-emitting layer through the second electrode, wherein the color conversion layer comprises the formulation.
